(19) United States
(12) Patent Application Publication
(10) Pub. No.: US 2024/0009321 A1
(43) Pub. Date: Jan. 11, 2024

(54) IMMUNOGENIC NANOVESICLES FOR CANCER IMMUNOTHERAPY

(71) Applicant: Arizona Board of Regents on Behalf of the University of Arizona, Tucson, AZ (US)

(72) Inventor: Jianqin Lu, Tucson, AZ (US)

(21) Appl. No.: 18/254,068

(22) PCT Filed: Nov. 23, 2021
(86) PCT No.: PCT/US2021/060642

§ 371 Date: May 23, 2023

Related U.S. Application Data
(60) Provisional application No. 63/117,629, filed on Nov. 24, 2020.

(51) Int. Cl. A61K 47/69; A61K 45/06; A61P 35/00

(52) U.S. Cl. CPC A61K 47/6911; A61K 45/06; A61P 35/00; B82Y 5/00

(57) ABSTRACT

Described herein are compositions and methods for treating cancer. The compositions comprise sphingomyelin-conjugated cancer drugs which can be formed into nanovesicles. These nanovesicles can be loaded with additional doxorubicin-conjugated drugs to provide combination therapeutics. These compositions are efficacious for cancer treatments.

### PRIORITY CLAIM

This application claims priority to U.S. Provisional Patent Application Ser. No. 63/117,629 filed Nov. 24, 2020, the entire contents of which are incorporated herein by reference.

### REFERENCE TO SEQUENCE LISTING

This application is filed with a Computer Readable Form of a Sequence Listing in accord with 37 C.F.R. § 1.821(c). The text file submitted by EFS, “212443-9011-US01_sequence_listing_24 Nov. 2020_ST25.txt,” was created on Nov. 24, 2020, contains 4 sequences, has a file size of 954 bytes, and is hereby incorporated by reference in its entirety. PGPubs, publish as is without CD. CRF file will be furnished by PTO.

### STATEMENT REGARDING FEDERALLY SPONSORED RESEARCH

This invention was made with government support under grant numbers P30 ES006694, R35 ES031575, P30 CA023074, and R01 CA092596 awarded by the National Institutes of Health (NIH). The United States government has certain rights in the invention.

### TECHNICAL FIELD

Described herein are compositions and methods for treating cancer. The compositions comprise sphingomyelin-conjugated cancer drugs which can be formed into nanovesicles. These nanovesicles can be loaded with additional doxorubicin (DOX)-conjugated drugs to provide combination therapeutics. These compositions are efficacious for cancer treatments.

### BACKGROUND

While immune checkpoint blockade (ICB) therapy (e.g., α-CTLA-4, α-PD-L1, α-PD-1) has transformed cancer treatment paradigm, only a subset of patients is responsive [1,2]. Against colorectal cancer (CRC) specifically, ICB is mostly ineffective—the exception being the ˜4% of patients with mismatch-repair-deficient or microsatellite instability-high tumors [3, 4]. Extensive efforts have centered on employing therapeutic modalities (e.g., chemotherapy, radiation/viral/targeted therapies, and therapeutic vaccine) that can turn “immune-cold” tumors into “immune-hot” to potentiate ICB immunotherapy [5-10]. Among which, immunogenic chemotherapy has shown remarkable potential to synergize with ICB (e.g., increasing tumor-infiltrated CTL). However, owing to the poor pharmacokinetics, limited tumor accumulation, and non-specific toxicities to healthy tissues/immune cells, chemotherapeutic utility in enhancing ICB's efficacy has been hindered.

Camptothecin (CPT), a potent anticancer chemotherapeutic against various cancers including CRC, has shown potential to enhance CTL-mediated tumor cells killing [11]. Nevertheless, the poor water solubility, severe adverse effects, and lactone ring instability limit CPT's clinical application and combination with ICB [12]. There is no FDA-approved CPT formulation, notwithstanding the extensive efforts made to overcome CPT's limitations.

There remains a need for a therapeutic platform capable of enhancing ICB therapeutic efficacy.

### SUMMARY

Sphingomyelin drug conjugates. DOX-drug conjugates and nanovesicles comprising the same, methods of preparing the same, and methods of treating and/or preventing cancer using the same are provided herein.

In some aspects, the present disclosure provides a sphingomyelin drug conjugate comprising Formula (I):

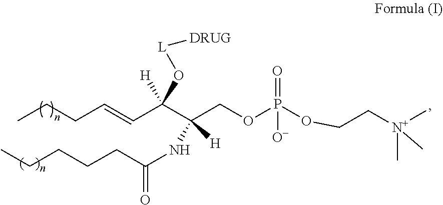

wherein each n is independently 5 to 20;
L is a linker moiety; and
Drug is an anti-cancer drug.

In other aspects, the present disclosure provides a doxorubicin (DOX)-drug conjugate comprising Formula (VII)-(VII):

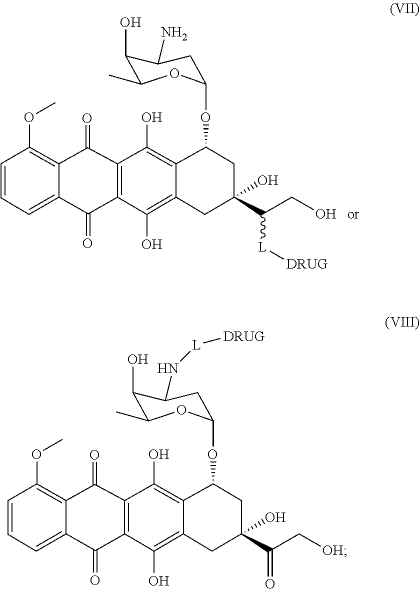

wherein:
   L is a linker moiety; and
   Drug is an anti-cancer drug.

In another aspect, the present disclosure provides a nanovesicle comprising a lipid bilayer including a sphingomyelin drug conjugate comprising Formula (I):

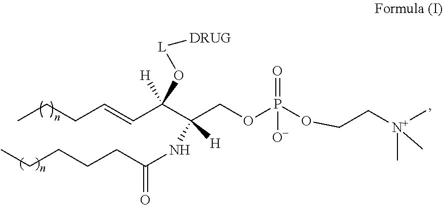

wherein each n is independently 5 to 20;
L is a linker moiety; and
Drug is an anti-cancer drug.

In some embodiments, the nanovesicle further comprises one or more DOX-drug conjugates in an interior core of the nanovesicle.

In some embodiments, a nanovesicle of the present disclosure is further conjugate to one or more tumor targeting ligands. In certain of these embodiments, the one or more tumor targeting ligands is selected from the group consisting of folate or folic acid, anisamide, phenylboronic acid, glycyrrhizic acid, pamidronic acid, triphenylphosphine, flavin mononucleotide; Polysaccharides: hyaluronic acid, galactose, chitosan, mannose, heparin, dextran, N-acetyl-β-D-galactosamine, sialic acid, lactobionic acid; Proteins: transferrin, EGFP-EGF1, AopB, ApoE, lactoferrin, tumor necrosis factor (TNF)-related apoptosis-inducing ligand (TRAIL); Antibodies: intercellular adhesion molecule 1 antibody (ICAM-1), CD44 antibody, EGFR antibody (cetuximab, panitumumab), PD-L1 antibody, EpCAM antibody, EphA10 antibody, AFP antibody, AMG655 antibody; Peptides: arginine-glycine-aspartate (RGD), asparagine-glycine-arginine (NGR), melittin (Mel), MT peptide, T7 peptide, Cell-penetrating peptides (CPP), Gly-Sar, mitochondria) targeting peptide (pALDH Leader), K237 peptide, YIGSR peptide, poly(histidine-arginine)6 (H6R6), angiopep-2, octreotide, pardaxin, Fragment C of tetanus toxin (TTC); Aptamers: aptamer S6, aptamer GBI-10, aptamer AS1411, aptamer RP, aptamer R8, aptamer AraHH036, aptamer MUC1, aptamer PSMA, aptamer EpCAM, and combinations thereof.

In some aspects, the present disclosure provides a method for preparing a sphingomyelin drug conjugate comprising a sphingomyelin, linker moiety, and an anti cancer drug, the method comprising: (a) providing the sphingomyelin, the linker moiety, and the anti-cancer drug; (b) conjugating the linker moiety to the anti-cancer drug to form an anti-cancer drug-linker moiety; and (c) conjugating the anti-cancer drug-linker moiety to the sphingomyelin to form the sphingomyelin drug conjugate.

In some embodiments, conjugating the anti-cancer drug-linker to the sphingomyelin occurs via a condensation reaction between the anti-cancer drug-linker and the sphingomyelin.

In another aspect, the present disclosure provides a method for preparing a DOX-drug conjugate comprising doxorubicin (DOX), a linker moiety, and an anti cancer drug, the method comprising: (a) providing the DOX, the linker moiety, and the anti-cancer drug; (b) conjugating the linker moiety to the anti-cancer drug to form an anti-cancer drug-linker moiety; and (c) conjugating the anti-cancer drug-linker moiety to the DOX to form the DOX-drug conjugate.

In some embodiments, conjugating the anti-cancer drug-linker to DOX occurs via a condensation reaction between the anti-cancer drug-linker and DOX.

In some aspects, the present disclosure provides a method for preparing a nanovesicle comprising a sphingomyelin-drug conjugate and a DOX-drug conjugate, the method comprising: (a) self-assembling the sphingomyelin-drug conjugate into a nanovesicle comprising a bilayer including the sphingomyelin-drug conjugate; (b) incubating the nanovesicle with the DOX-drug conjugate, wherein the DOX-drug conjugate enters into an interior core of the nanovesicle to form the nanovesicle comprising the sphingomyelin-drug conjugate and the DOX-drug conjugate.

In some embodiments, DOX-drug conjugate comprises DOX, a linker, and a drug, wherein the drug of the DOX-drug conjugate precipitates from the DOX-drug conjugate, releasing both DOX and the drug into the interior core of the nanovesicle. In certain embodiments, the sphingomyelin-drug conjugate is self-assembled as a thin film. In some embodiments, the method further comprises combining the nanovesicle comprising a bilayer including the sphingomyelin-drug conjugate with a transmembrane agent prior to step (b).

In some embodiments, the method further comprises sonicating the nanovesicle and the transmembrane agent. In certain of these embodiments, the transmembrane gradient agent comprises one or more of citric acid, triethylammonium sucrose octasulfate (TEA8SOS), ammonium salts, e.g., ammonium sulfate, ammonium α-cyclodextrin sulfate, ammonium sucrose octasulfate, ammonium phosphate, ammonium β-cyclodextrin sulfate, ammonium β-cyclodextrin phosphate, ammonium γ-cyclodextrin sulfate, ammonium γ-cyclodextrin phosphate, ammonium α-cyclodextrin phosphate, ammonium acetate, or ammonium citrate; trimethylammonium salts, e.g., trimethylammonium sucrose octasulfate, trimethylammonium sulfate, trimethylammonium α-cyclodextrin sulfate, trimethylammonium γ-cyclodextrin sulfate, trimethylammonium α-cyclodextrin phosphate, trimethylammonium β-cyclodextrin sulfate, trimethylammonium γ-cyclodextrin phosphate, trimethylammonium phosphate, trimethylammonium β-cyclodextrin phosphate, trimethylammonium citrate, or trimethylammonium acetate; or triethylammonium salts, e.g., triethylammonium sulfate, triethylammonium γ-cyclodextrin sulfate, triethylammonium α-cyclodextrin sulfate, triethylammonium β-cyclodextrin sulfate, triethylammonium phosphate, triethylammonium β-cyclodextrin phosphate, triethylammonium α-cyclodextrin phosphate, triethylammonium γ-cyclodextrin phosphate, triethylammonium acetate, or triethylammonium citrate; or combinations thereof.

In some embodiments, the DOX-drug conjugate is incubated with the nanovesicle comprising a bilayer including the sphingomyelin-drug conjugate for a period of time and/or temperature sufficient to incorporate the DOX-drug conjugate into the interior core of the nanovesicle. In certain of these embodiments, the period of time is 30 min to 90 minutes and the temperature is 50° C. to 70° C.

In some embodiments, the method further comprises conjugating one or more tumor targeting ligands to the nanovesicle. In certain of these embodiments, the tumor targeting ligand comprises one or more small molecule selected from: folate or folic acid, anisamide, phenylboronic acid, glycyrrhizic acid, pamidronic acid, triphenylphosphine, flay in mononucleotide; Polysaccharides: hyaluronic acid, galactose, chitosan, mannose, heparin, dextran, N-acetyl-β-D-galactosamine, sialic acid, lactobionic acid; Proteins: transferrin, EGFP-EGF1, AopB, ApoE, lactoferrin, tumor necrosis factor (TN F)-related apoptosis-inducing ligand (TRAIL); Antibodies: intercellular adhesion molecule 1 antibody (ICAM-1), CD44 antibody, EGFR antibody (cetuximab, panitumumab), PD-L1 antibody, EpCAM antibody, EphA10 antibody, AFP antibody, AMG655 antibody; Peptides: arginine-glycine-aspartate (RGD), asparagine-glycine-arginine (NGR), melittin (Mel), MT peptide, T7 peptide, Cell-penetrating peptides (CPP), Gly-Sar, mitochondria) targeting peptide (pALDH Leader), K237 peptide, YIGSR peptide, poly(histidine-arginine)6 (H6R6), angiopep-2, octreotide, pardaxin, Fragment C of tetanus toxin (TTC); Aptamers: aptamer S6, aptamer GBI-10, aptamer AS1411, aptamer RP, aptamer R8, aptamer AraHH036, aptamer MUC1, aptamer PSMA, aptamer EpCAM, or combinations thereof.

In another aspect, the present disclosure provides a method of treating and/or preventing cancer in a subject in need thereof, the method comprising administering to the subject a nanovesicle comprising a sphingomyelin drug conjugate, wherein the sphingomyelin drug conjugate comprises Formula (I):

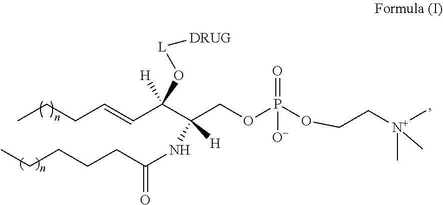

wherein each n is independently 5 to 20;
L is a linker moiety; and
Drug is an anti-cancer drug.

In some aspects, the present disclosure provides a method of treating and/or preventing cancer, the method comprising administering a nanovesicle comprising a sphingomyelin drug conjugate and a DOX-drug conjugate, wherein the sphingomyelin drug conjugate is incorporated into a bilayer of the nanovesicle and the DOX-drug conjugate is incorporated into an interior core of the nanovesicle.

In some embodiments, the cancer is adrenal cancer, anal cancer, basal and squamous cell skin cancer, bile duct cancer, bladder cancer, bone cancer, brain and spinal cord tumors (e.g., astrocytoma, glioblastoma multiforme, meningioma), breast cancer, cervical cancer, colorectal cancer, endometrial cancer, esophagus cancer, Ewing family of tumors, eye cancer (ocular melanoma), gallbladder cancer, gastrointestinal neuroendocrine (carcinoid) tumors, gastrointestinal stromal tumor (gist), gestational trophoblastic disease, Kaposi sarcoma, kidney cancer, laryngeal and hypopharyngeal cancer, liver cancer, lung cancer, lung carcinoid tumor, malignant mesothelioma, melanoma skin cancer, Merkle cell skin cancer, nasal cavity and paranasal sinuses cancer, nasopharyngeal cancer, neuroblastoma, non-small cell lung cancer, neoplasm of the central nervous system (CNS), oral cavity and oropharyngeal cancer, osteosarcoma, ovarian cancer, pancreatic cancer, pancreatic neuroendocrine tumor (net), penile cancer, pituitary tumors, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer, uterine sarcoma, vaginal cancer, vulvar cancer, Waldenstrom macroglobulinemia, Wilms tumor, squamous cell cancer, cancers of unknown primary (CUP), environmentally induced cancers, combinations of the cancers, and metastatic lesions of the cancers. In some embodiments, the cancer is leukemia or lymphoma, for example, lymphoblastic lymphoma or B-cell Non-Hodgkin's lymphoma.

In some embodiments, the cancer is a hematologic malignancy. In some embodiments, the hematologic malignancy is chronic lymphocytic leukemia (CLL), acute leukemia, acute lymphoid leukemia (ALL), B-cell acute lymphoid leukemia (B ALL), T-cell acute lymphoid leukemia (T-ALL), T-cell lymphoma, B-cell lymphoma, chronic myelogenous leukemia (CML), acute myelogenous leukemia, B-cell prolymphocytic leukemia, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia. In other embodiments, the cancer is a human hematologic malignancy such as myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplastic syndromes, lymphoid neoplasm s, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In some embodiments, the cancer comprises a solid tumor. In certain of these embodiments, the solid tumor selected from the group consisting of lung cancer, colorectal cancer, breast cancer, pancreatic cancer, gallbladder cancer, brain and spinal cord cancer, head and neck cancer, skin cancers, testicular cancer, prostate cancer, ovarian cancer, renal cell carcinoma (RCC), bladder cancer. and hepatocellular carcinoma (HCC).

In certain embodiments, the nanovesicles are present in a pharmaceutical composition.

In some embodiments, the sphingomyelin drug conjugate comprises Formula (I):

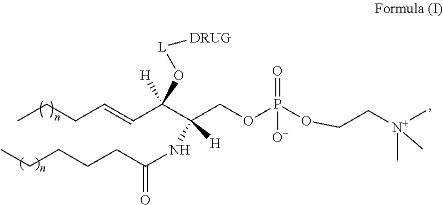

wherein each n is independently 5 to 20;
L is a linker moiety; and
Drug is an anti-cancer drug.

In some embodiments, the sphingomyelin drug conjugate comprises Formula (II)

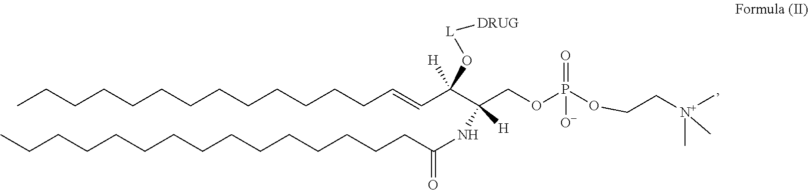

wherein L is a linker moiety; and
Drug is an anti-cancer drug.

In some embodiments, the sphingomyelin drug conjugate comprises an anti cancer drug that is hydrophilic or hydrophobic. In certain of these embodiments, the anti-cancer drug comprises a functional group selected from —COOH, —OH, —NH₂ and/or C═O.

In some embodiments, the sphingomyelin drug conjugate comprises an anti cancer drug selected from the group consisting of: cam ptothecin, paclitaxel, docetaxel, ADU-S100, amrubicin, 5-aminolevulinic acid, AZD4635, BMS-1001, BMS-1166, BMS-200, BMS-202, BMS-242, BMS-242, bortezomib, CA170, cabazitaxel, cabozantinib, canertinib, capecitabine, carboplatin, ceritinib, chlorin e6, cisplatin, dabrafenib, dacarbazine, darolutamide, daunorubicin, degarelix, digoxin, doxorubicin, epacadostat, epirubicin, eribulin, esorubicin, etoposide, fingolimod, 5-fluorouracil, galanthamine, gemcitabine, idarubicin, imatinib, imiquimod, indoximod, irinotecan, ixabepilone, lenvatinib, memantine, methotrexate, mitoxantrone, NIR178, NLG919, oxaliplatin, pazopanib, pemetrexed, preladenant, protoporphyrin IX (PPIX), pyropheophorbide-A (PPA), septacidin, SN-38, sorafenib, streptozocin, sunitinib, temozolomide, tipiracil, TPI-287, trifluridine, vadimezan, vemurafenib, vinblastine, vincristine, vinorelbine, vipadenant, vorinostat, and combinations thereof.

In some embodiments, the sphingomyelin drug conjugate comprises Formula (III)-(VI):

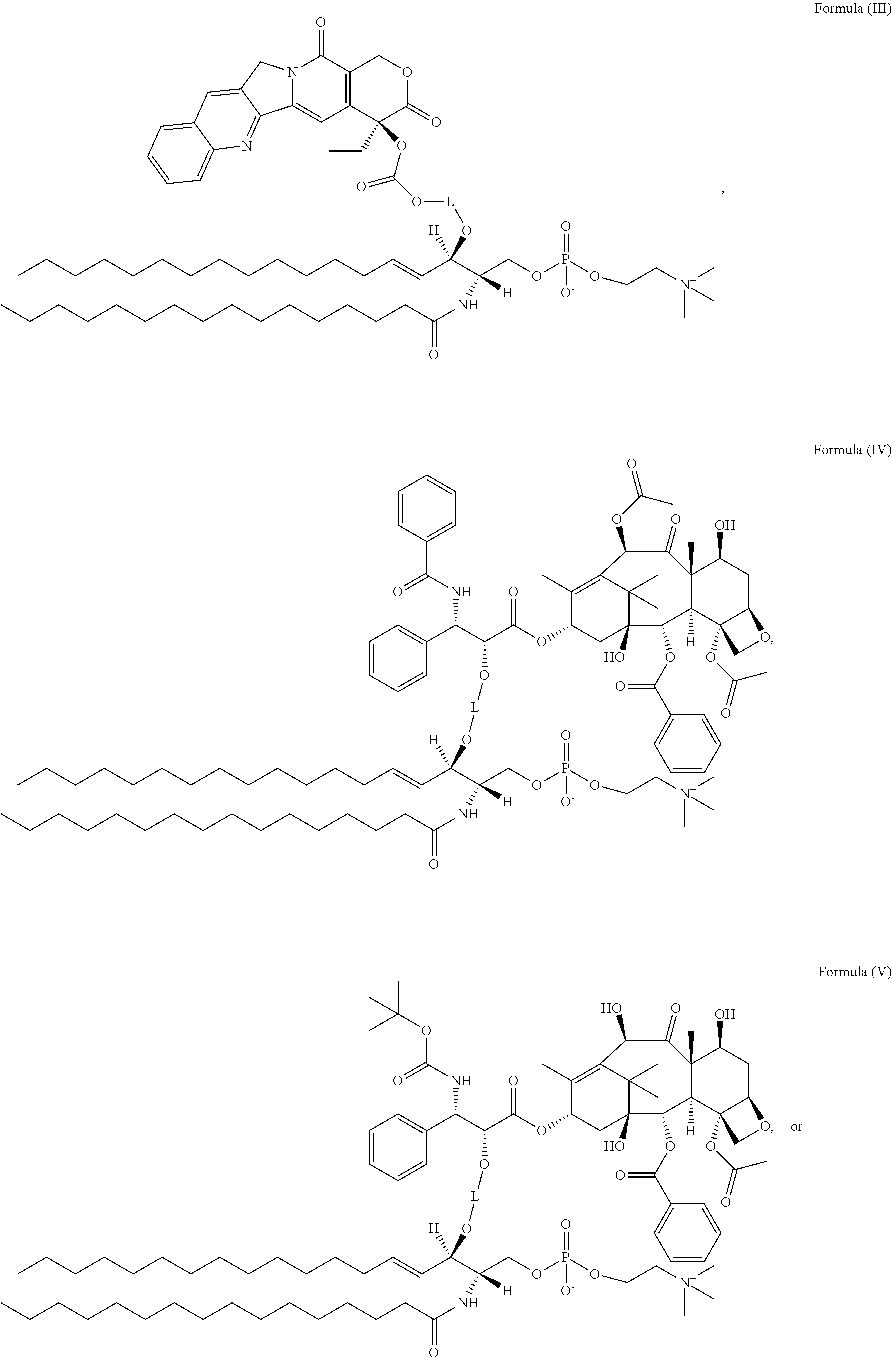

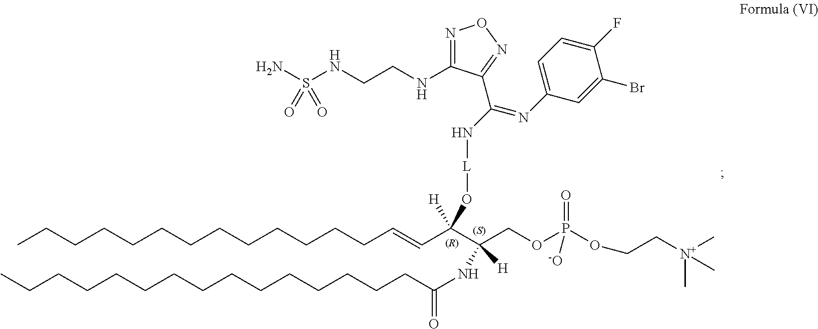

wherein L is a linker moiety.

In some embodiments, the sphingomyelin drug conjugate is:

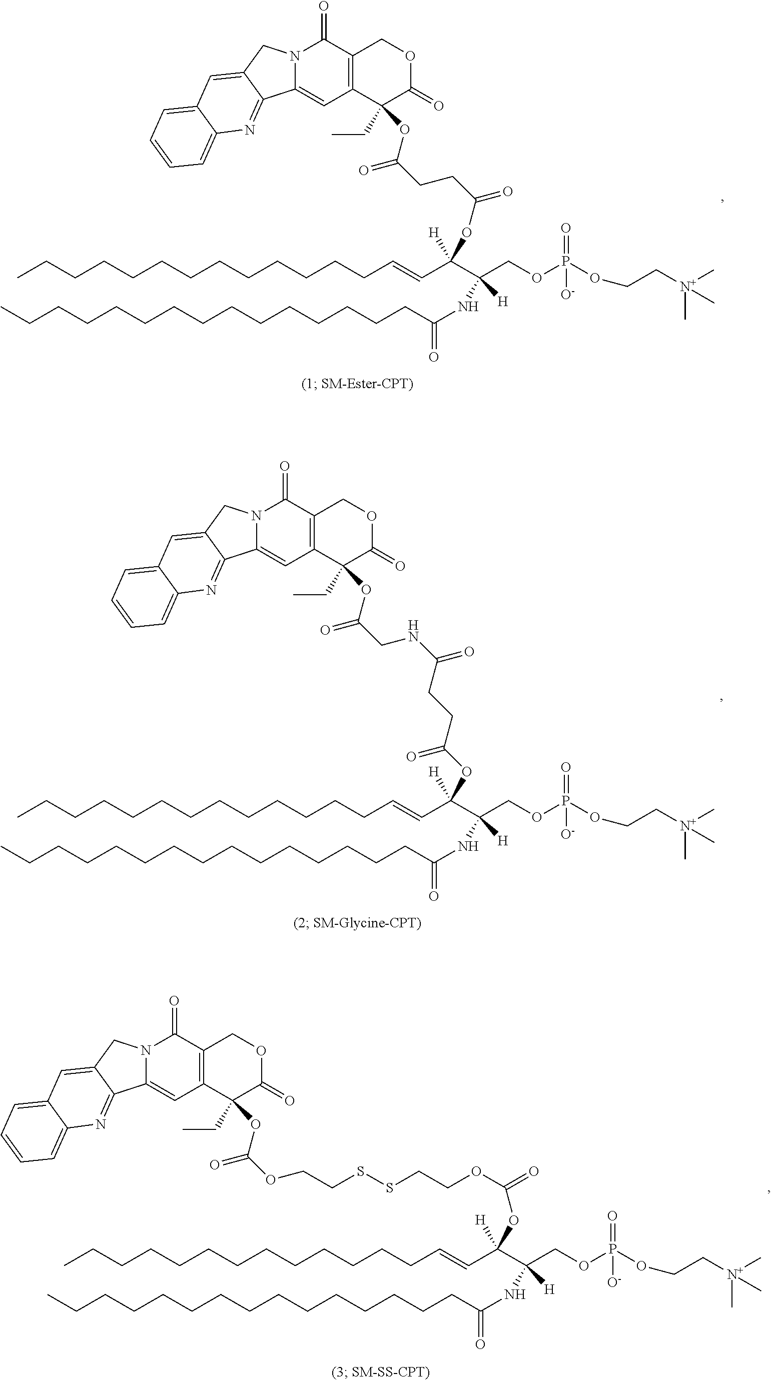

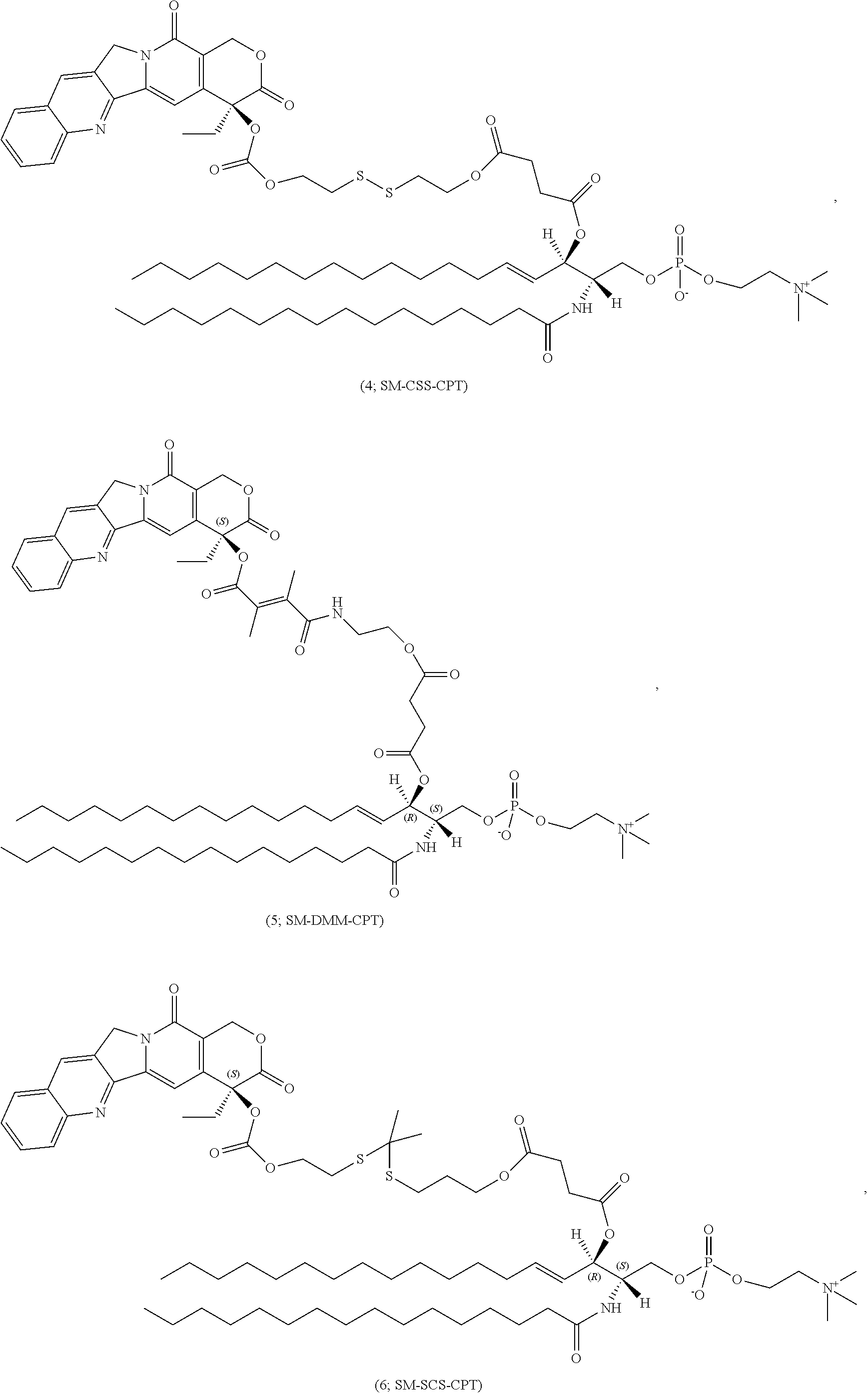

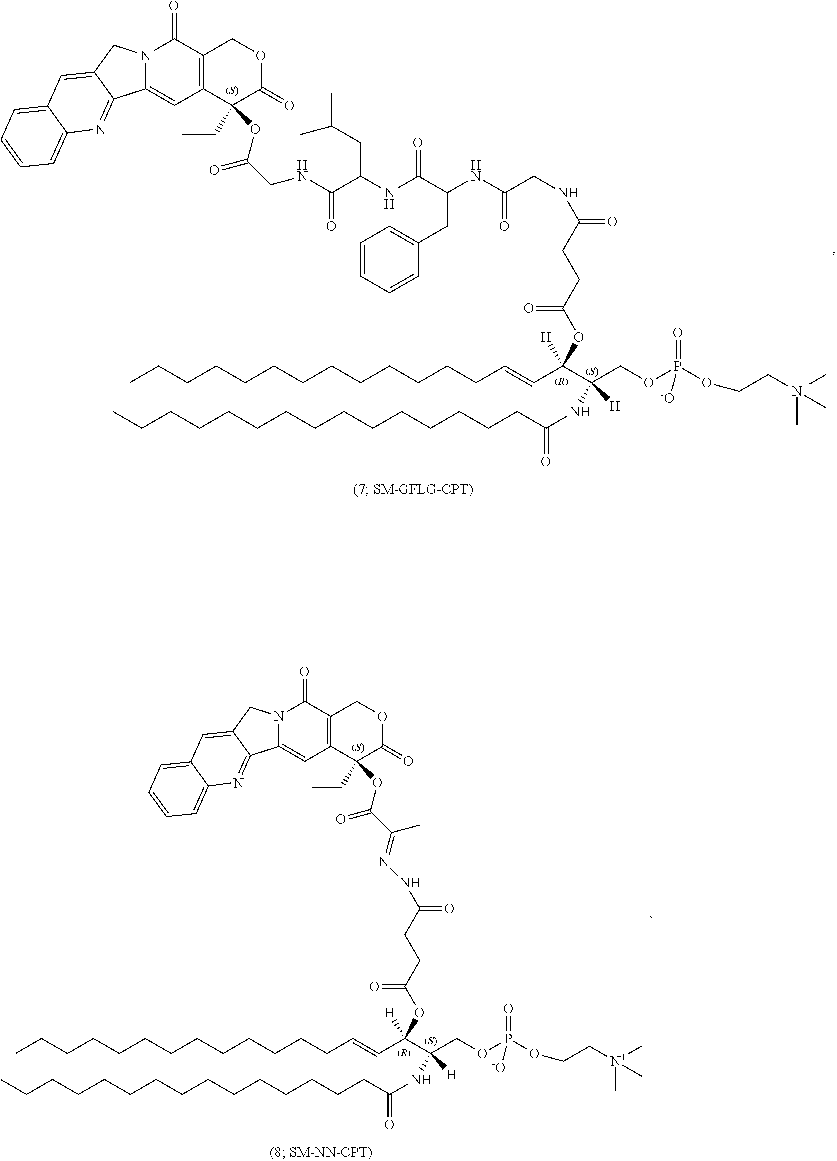

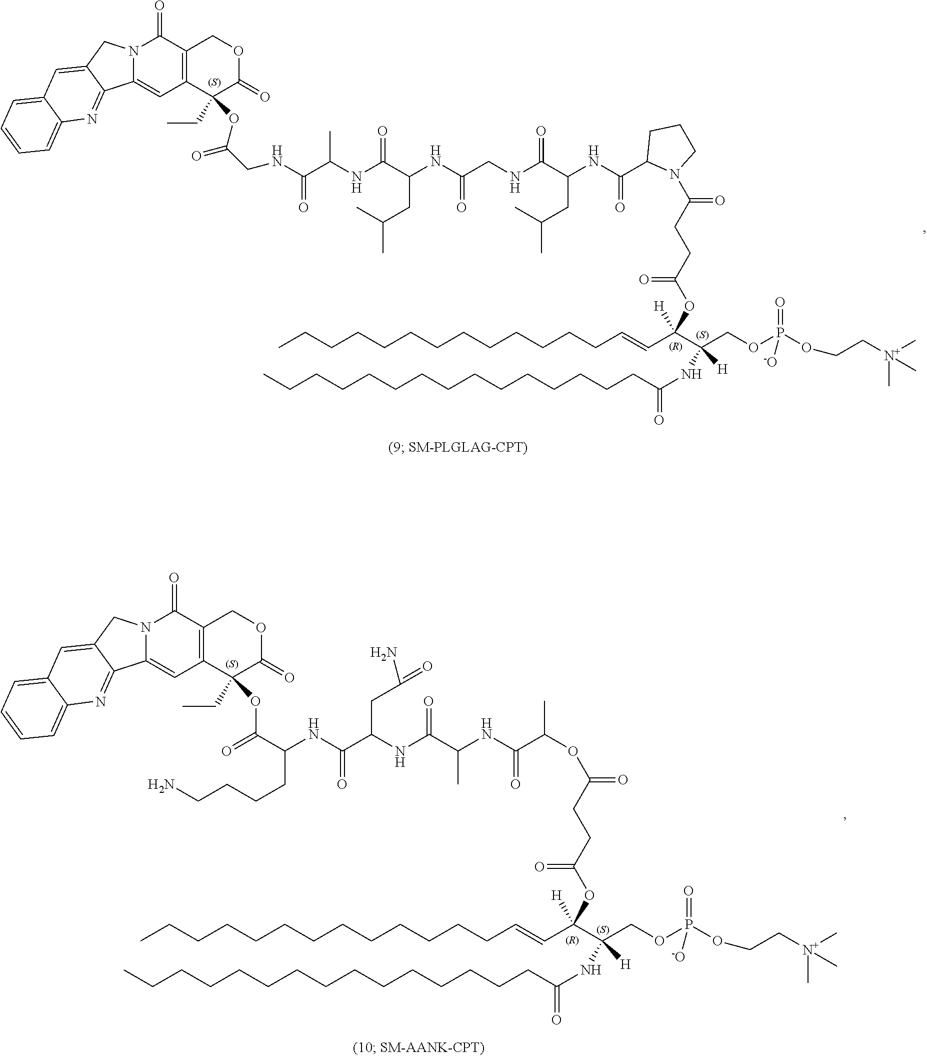

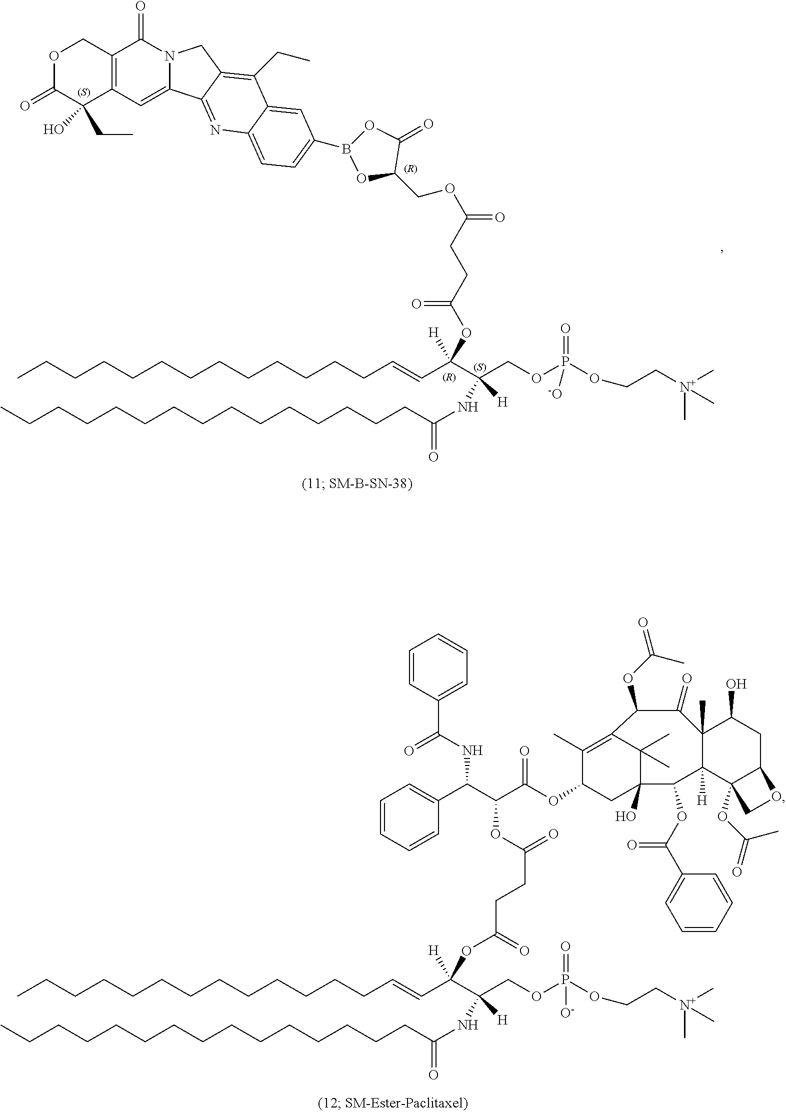

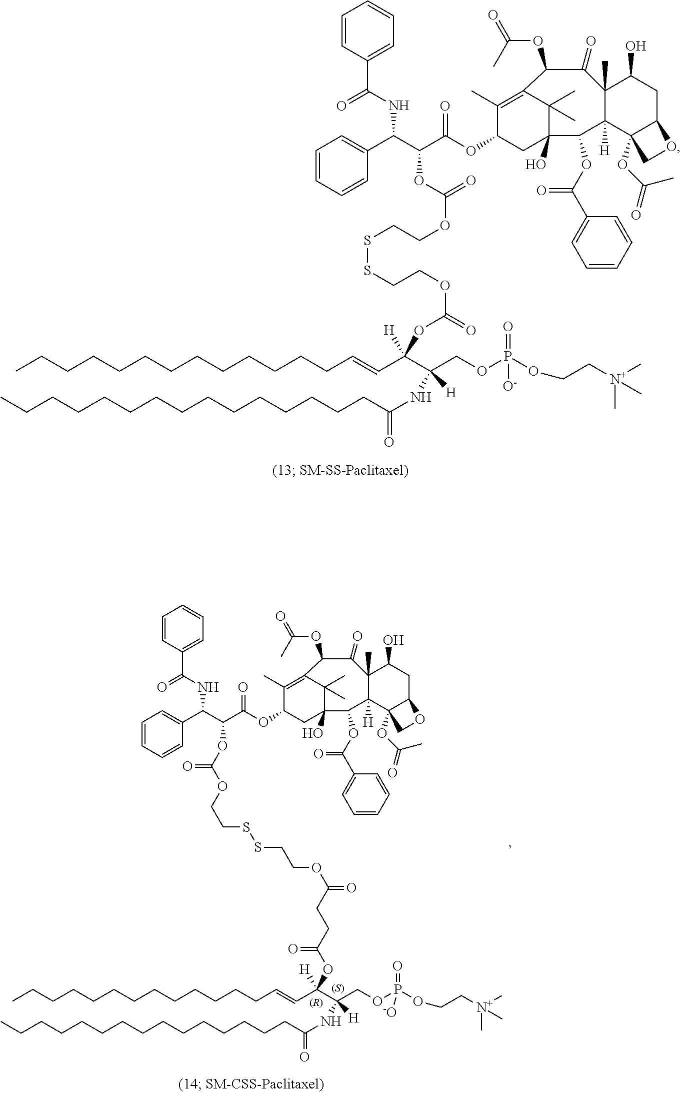

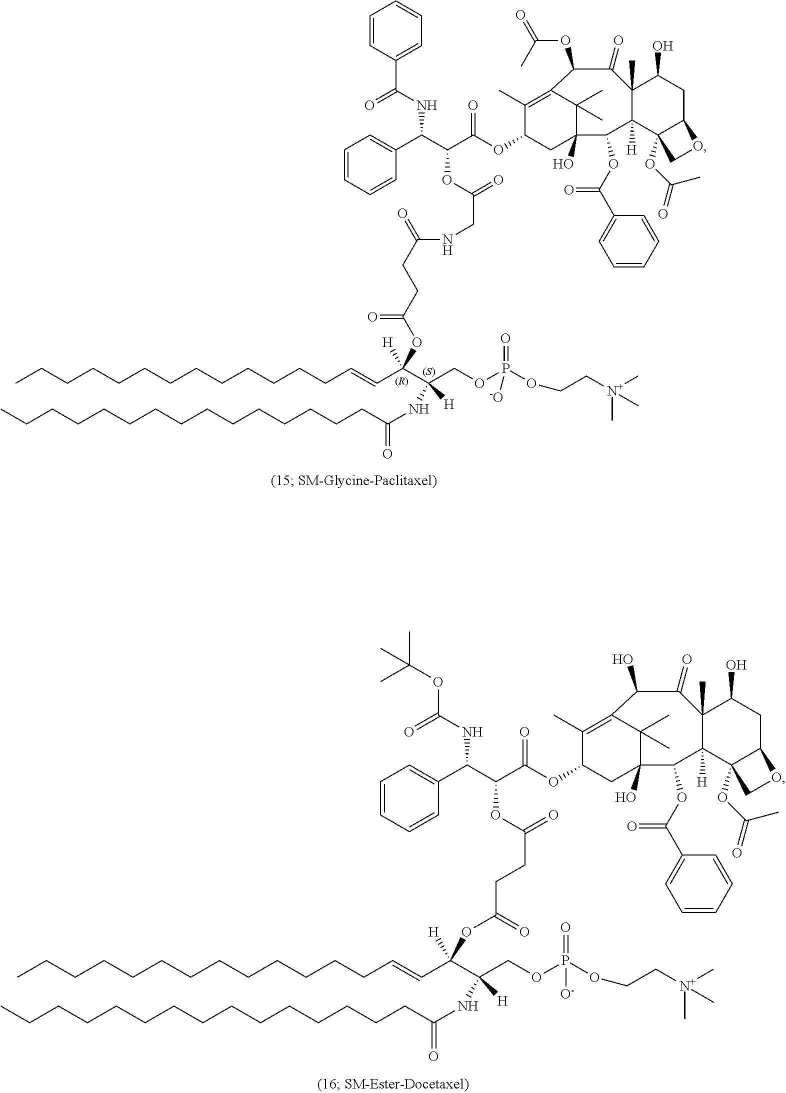

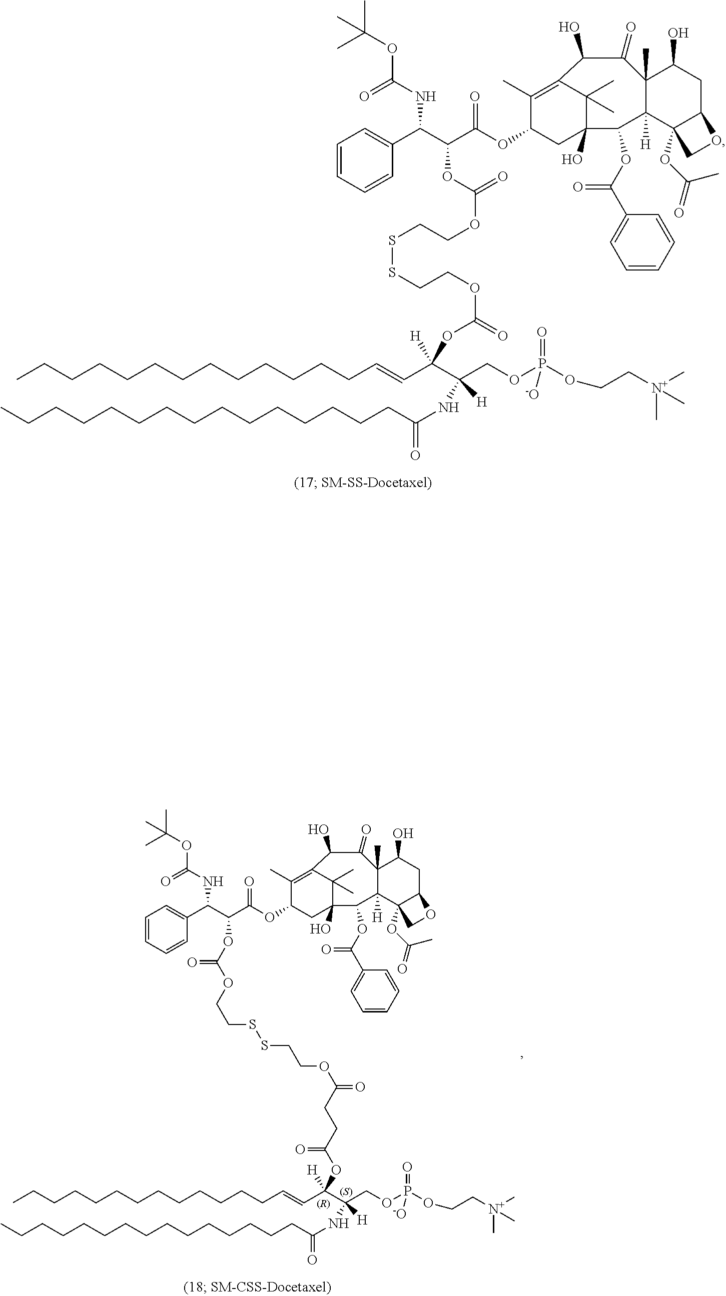

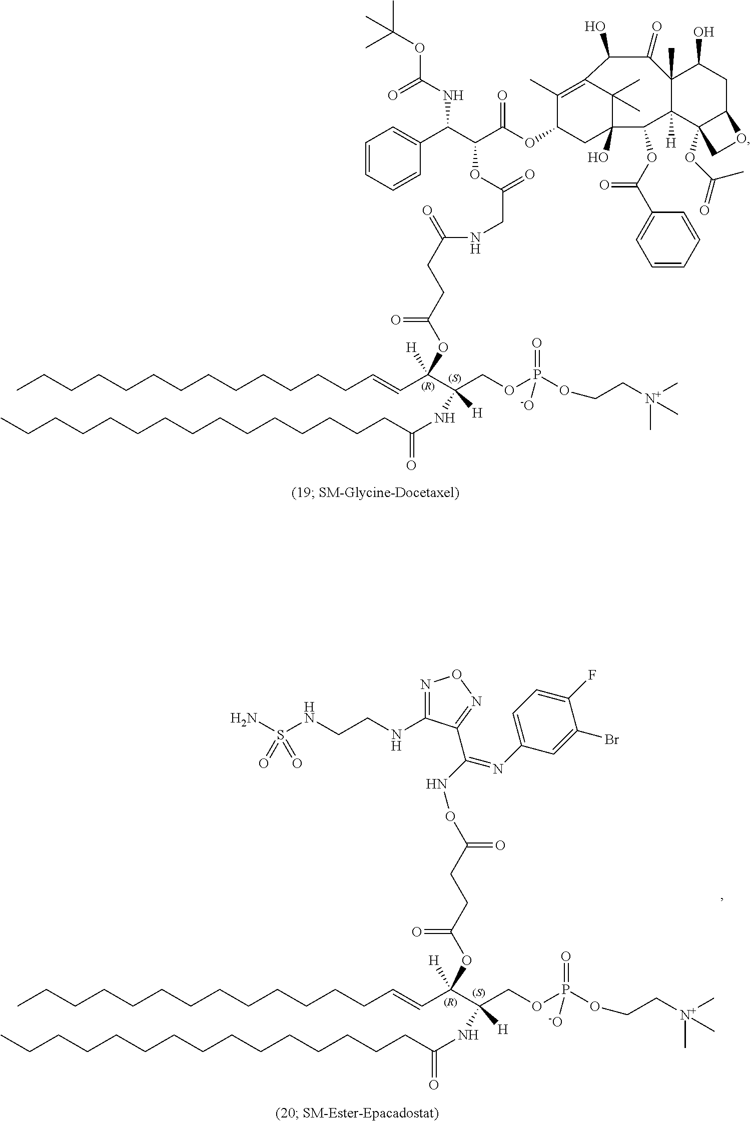

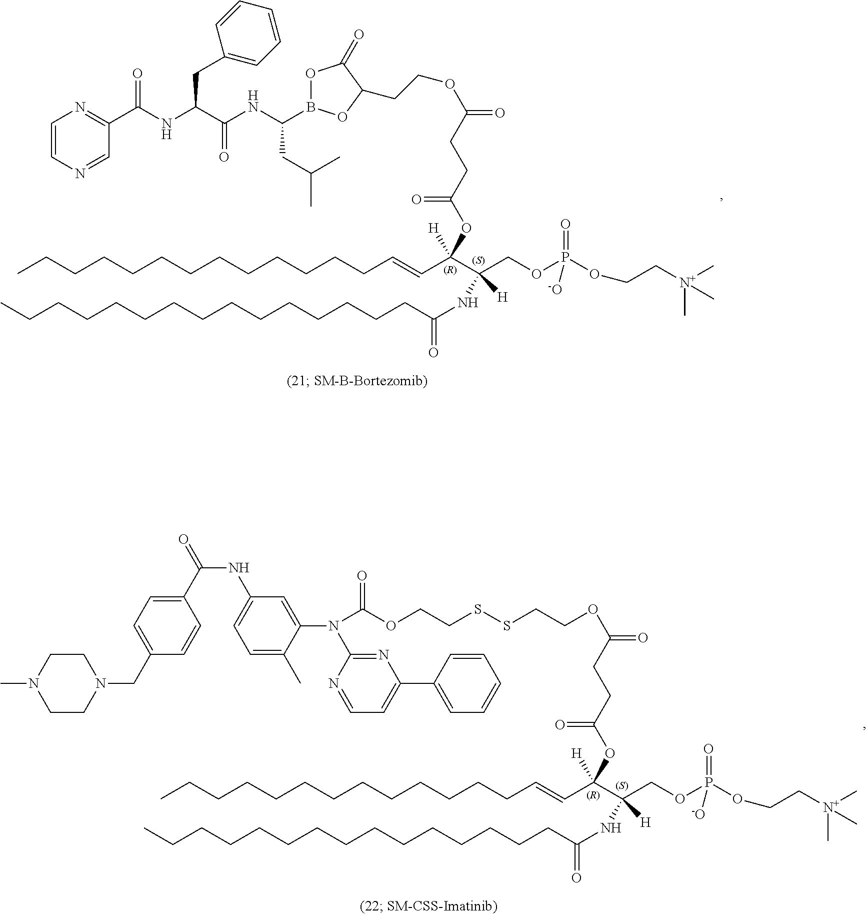

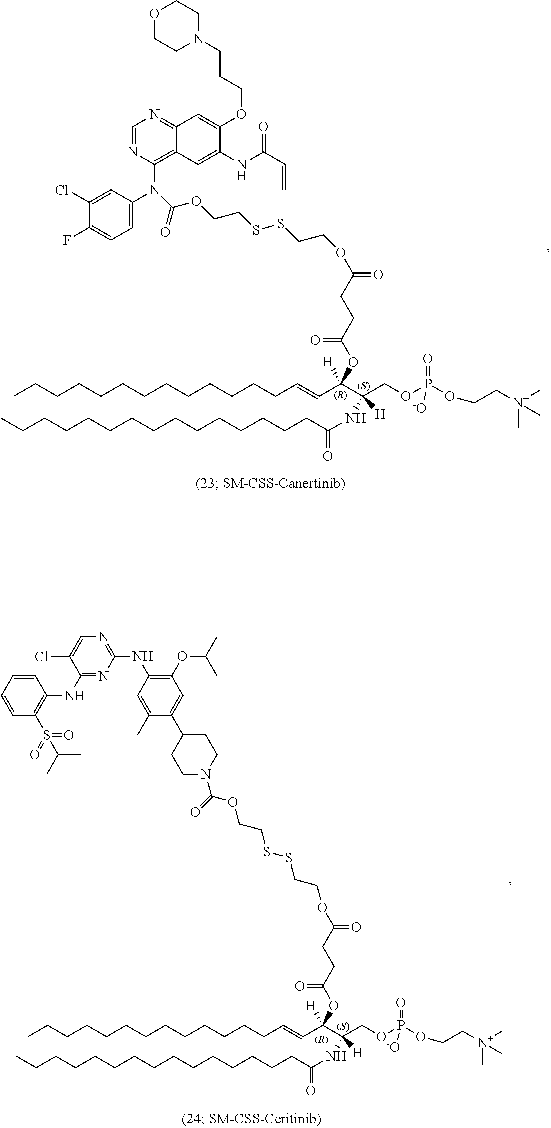

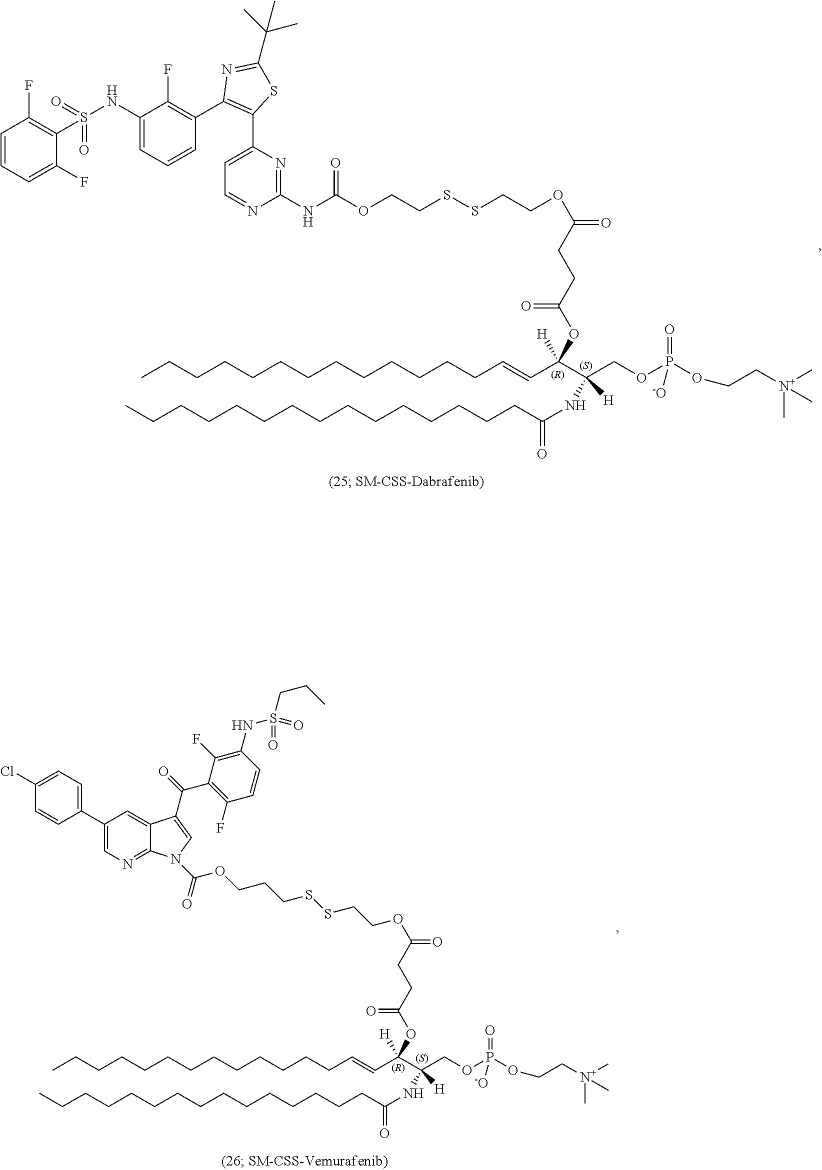

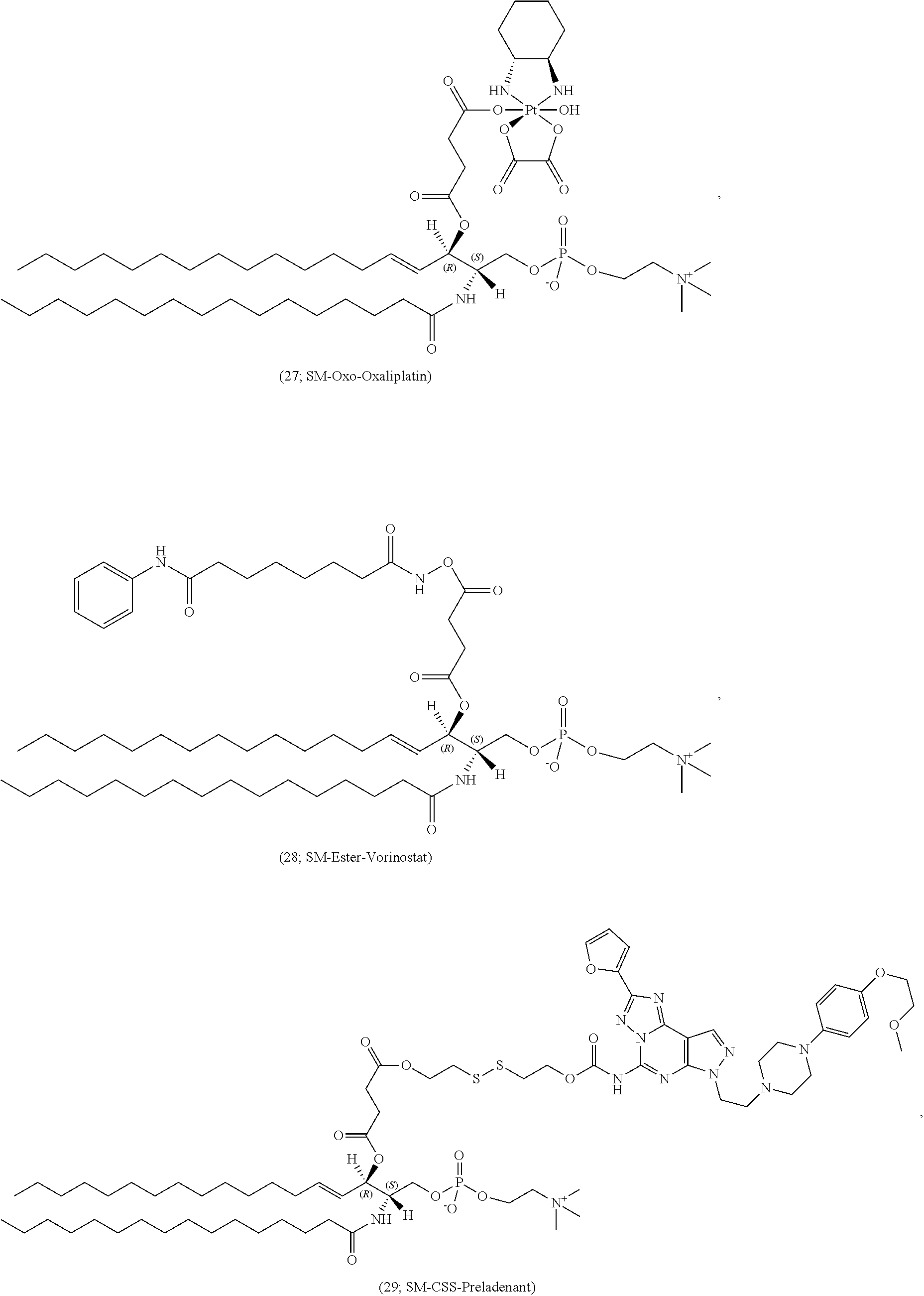

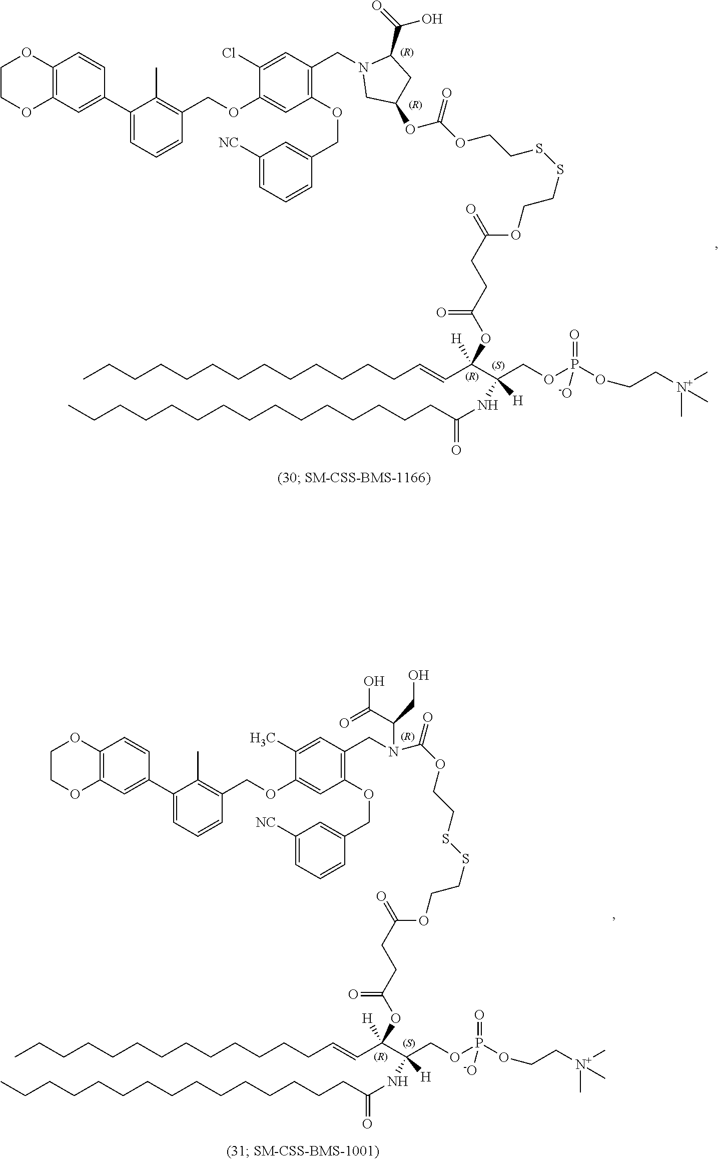

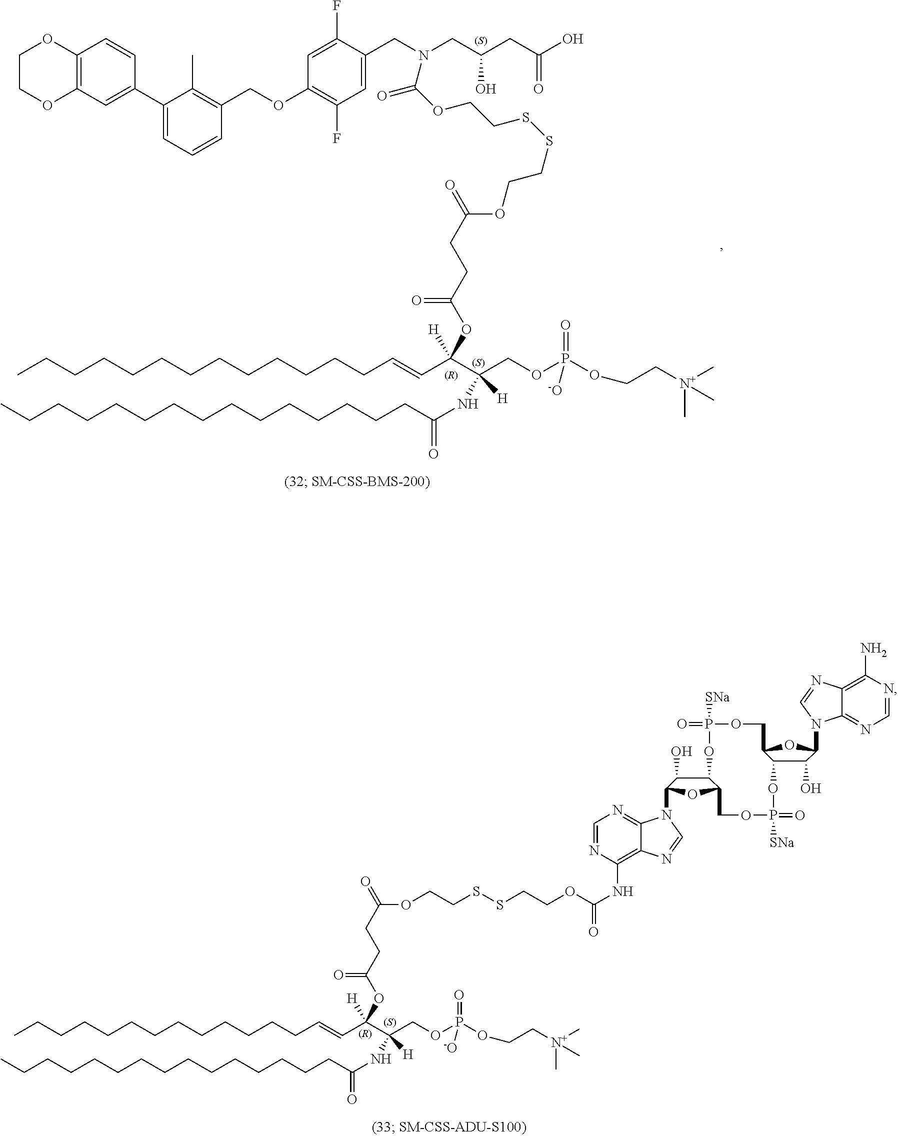

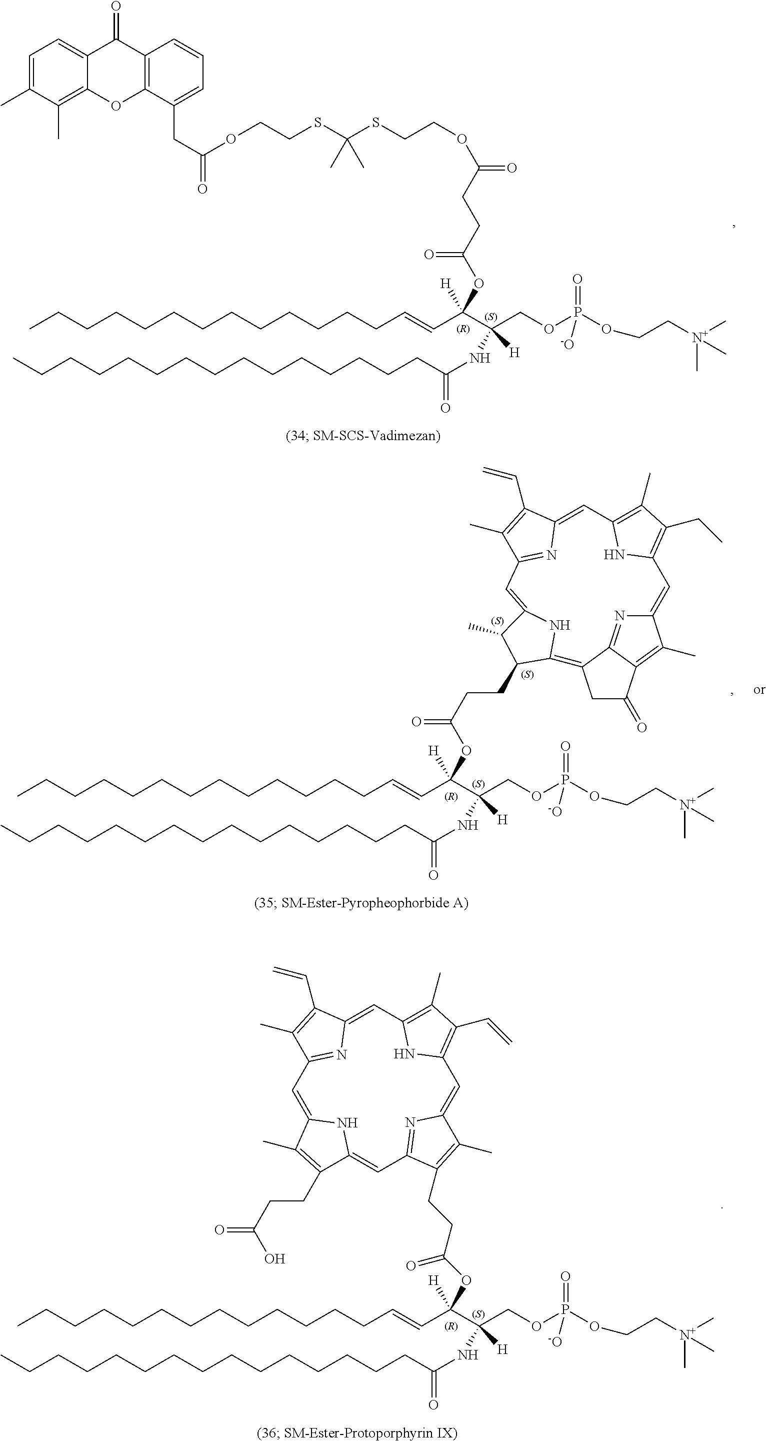

In some embodiments, the DOX-drug conjugate comprises an anti-cancer drug that is hydrophobic or hydrophobic. In certain of these embodiments, the anti cancer drug comprises a functional group selected from —COOH, —OH, —NH₂ and/or C═O.

In some embodiments, the DOX-drug conjugates comprises an anti-cancer drug selected from: indoximod, bortezomib, epacadostat, imiquimod, imatinib, canertinib, ceritinib, dabrafenib, vemurafenib, vorinostat, ADU-S100, amrubicin, AZD4635, BMS-1001, BMS-1166, BMS-200, BMS202, BMS-242, CA170, cabazitaxel, cabozantinib, cam ptothecin, capecitabine, carboplatin, cisplatin, dacarbazine, darolutamide, degarelix, digitoxin, digoxin, docetaxel, eribulin, etoposide, 5-fluorouracil, gem citabine, irinotecan, ixabepilone, lenvatinib, methotrexate, mitoxantrone, NIR178, NLG919, oxaliplatin, paclitaxel, pazopanib, pemetrexed, preladenant, septacidin, SN-38, sorafenib, streptozocin, sunitinib, temozolomide, tipiracil, trifluridine, vadimezan, vinblastine, vincristine, vinorelbine, vipadenant, or combinations thereof.

In certain embodiments, the DOX-drug conjugate comprises Formula (IX)-(XVIII):

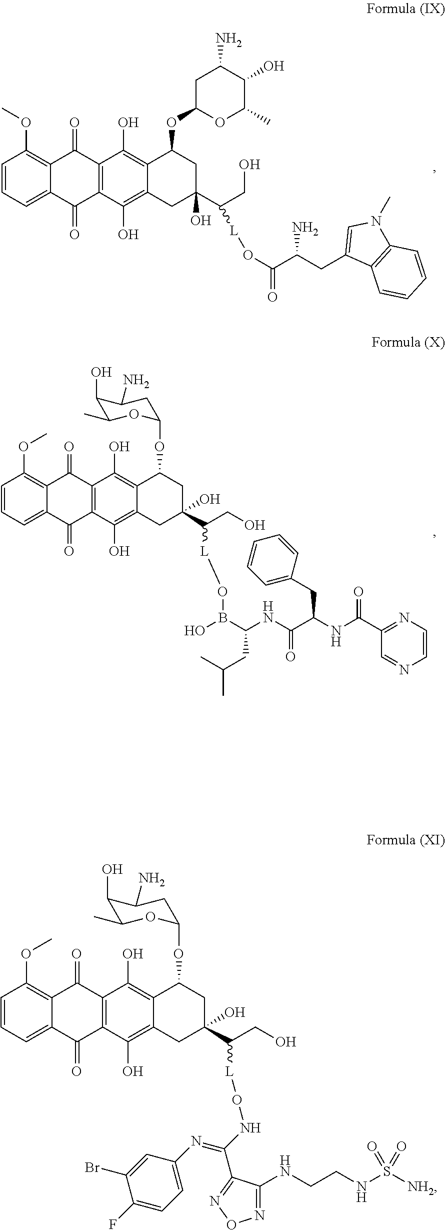

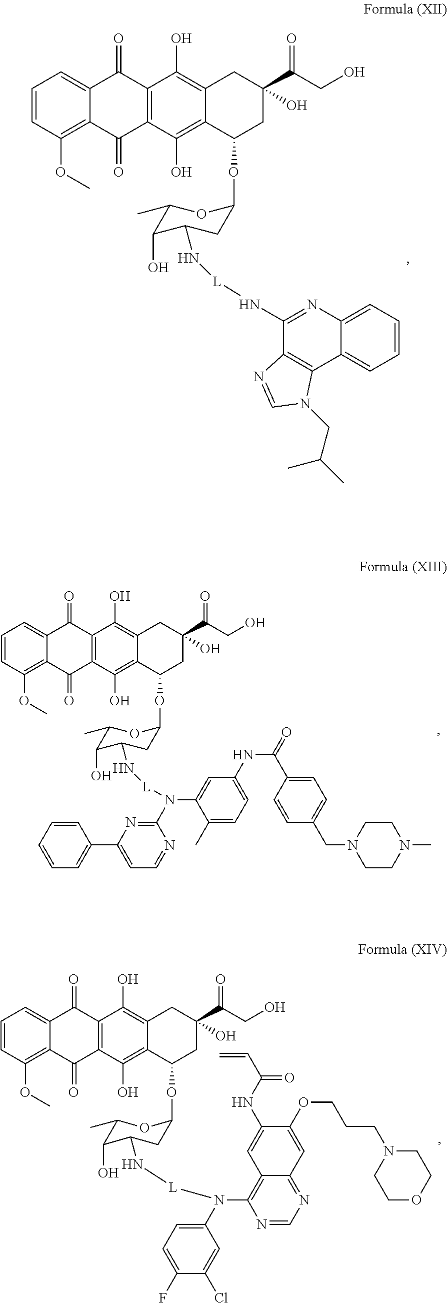

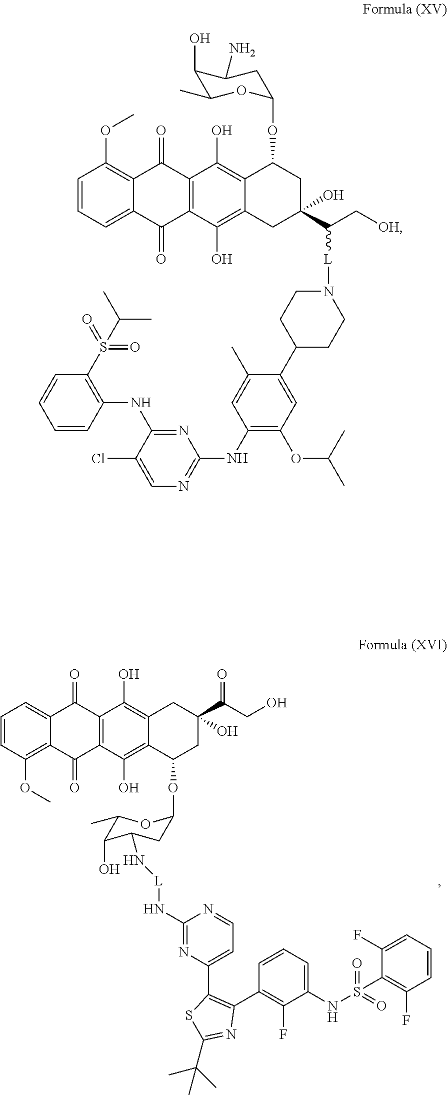

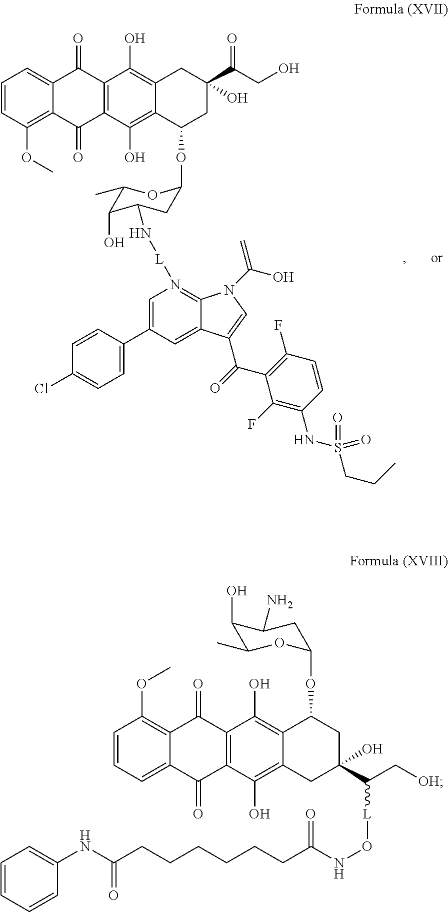

wherein L is a linker moiety.

In certain embodiments, L of the sphingomyelin drug conjugate and/or DOX-drug conjugate is selected from:

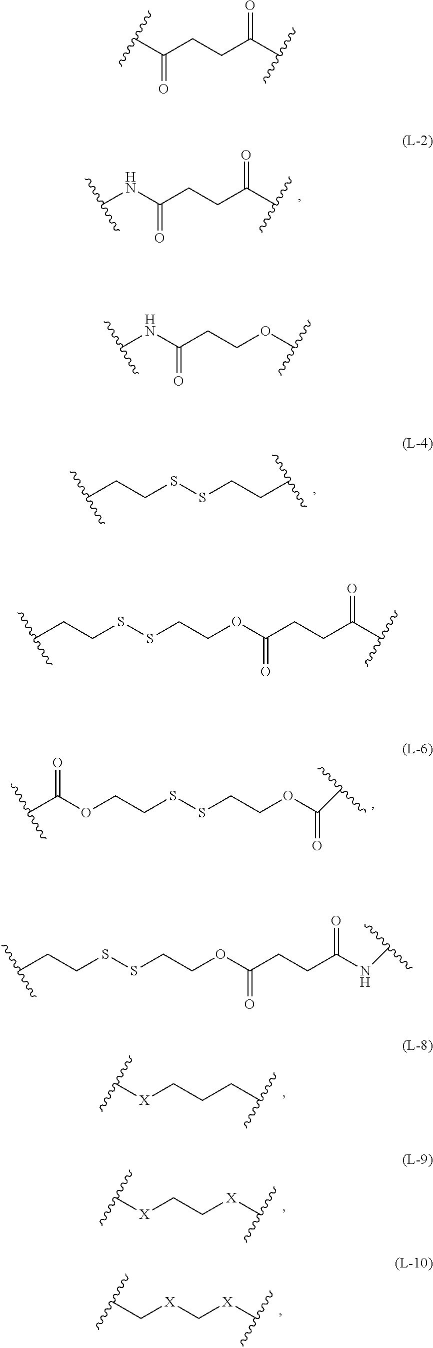

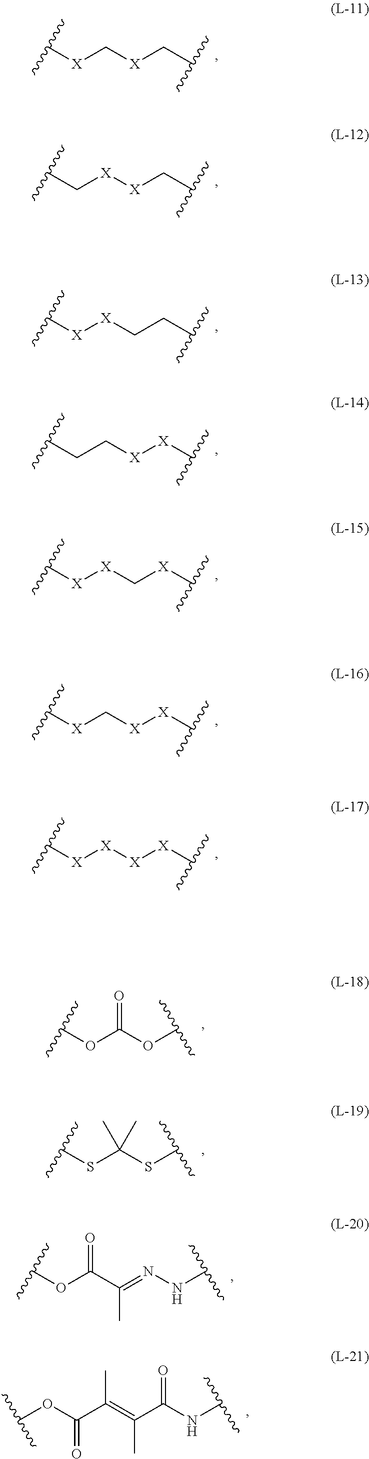

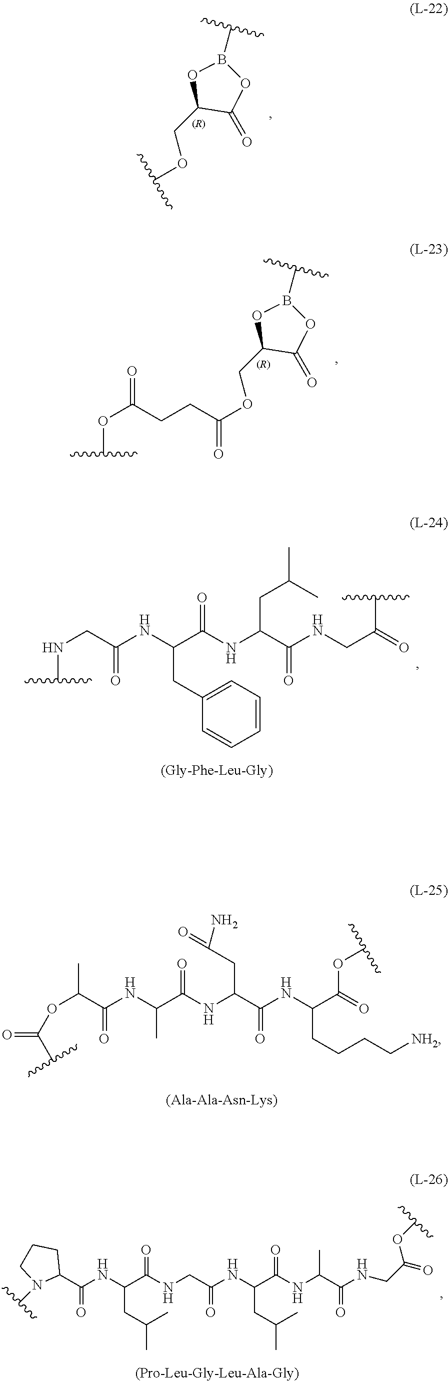

or combinations thereof;
wherein X is independently, O, S, —NH, or —CO.

In some embodiments, the DOX-drug conjugate is:

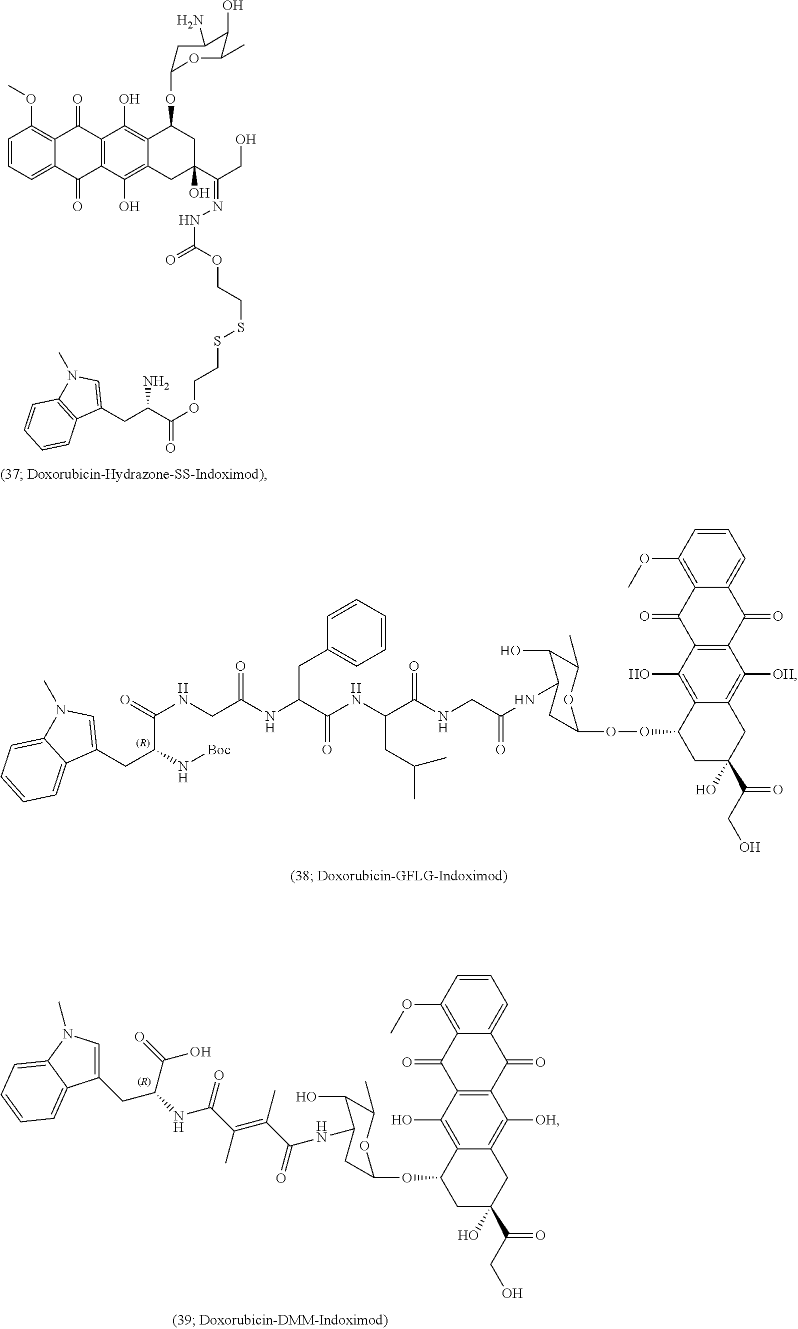

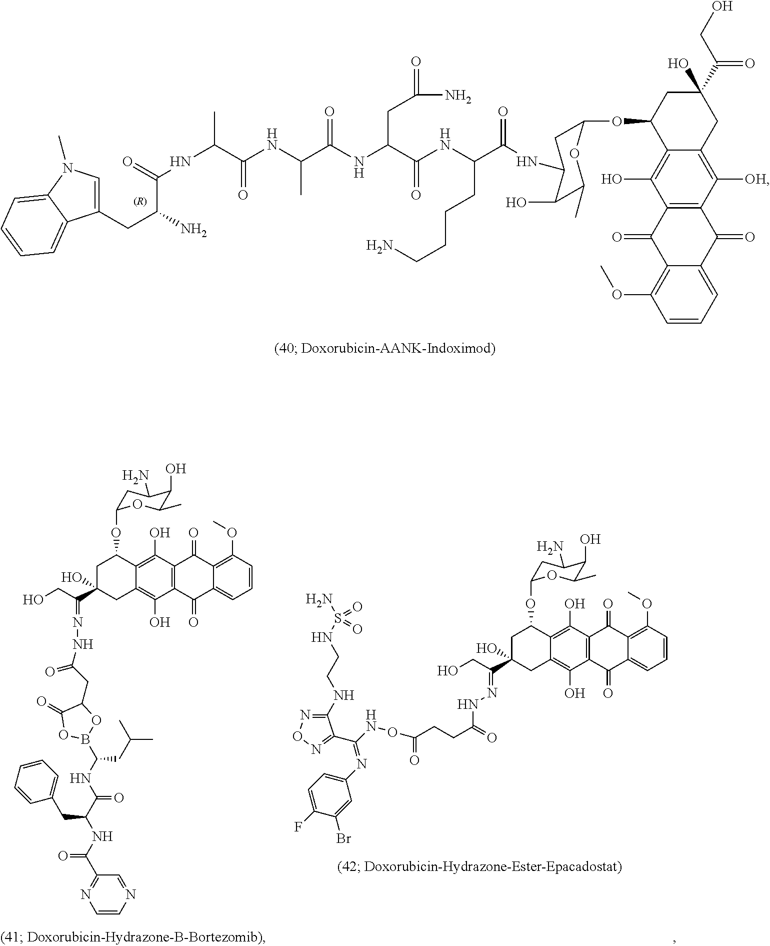

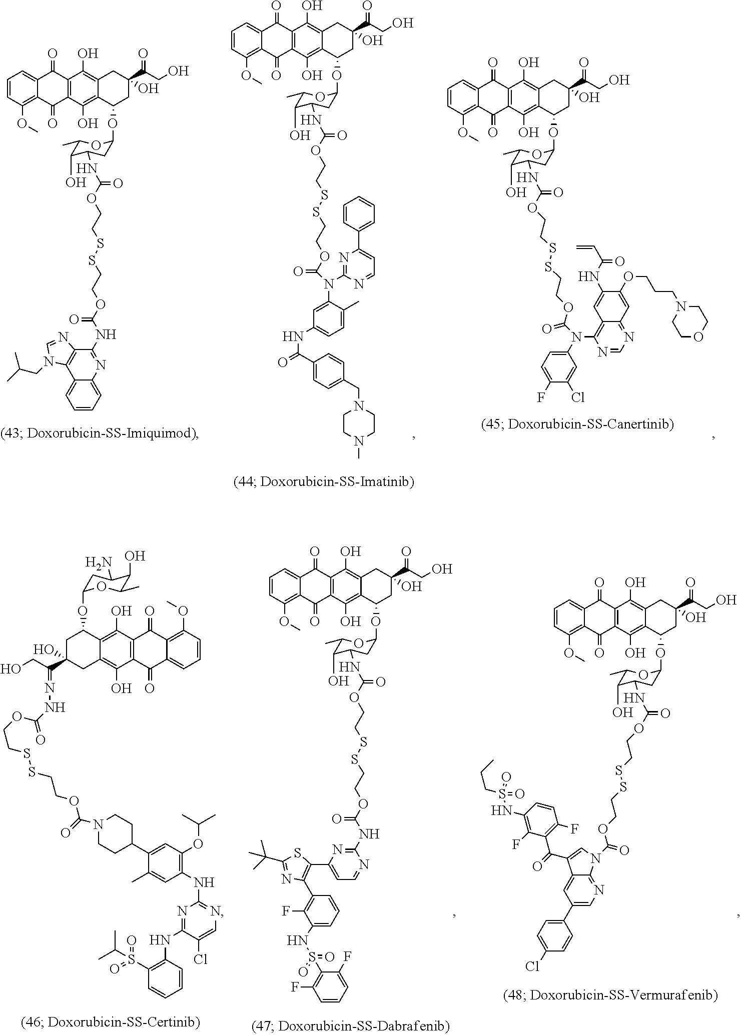

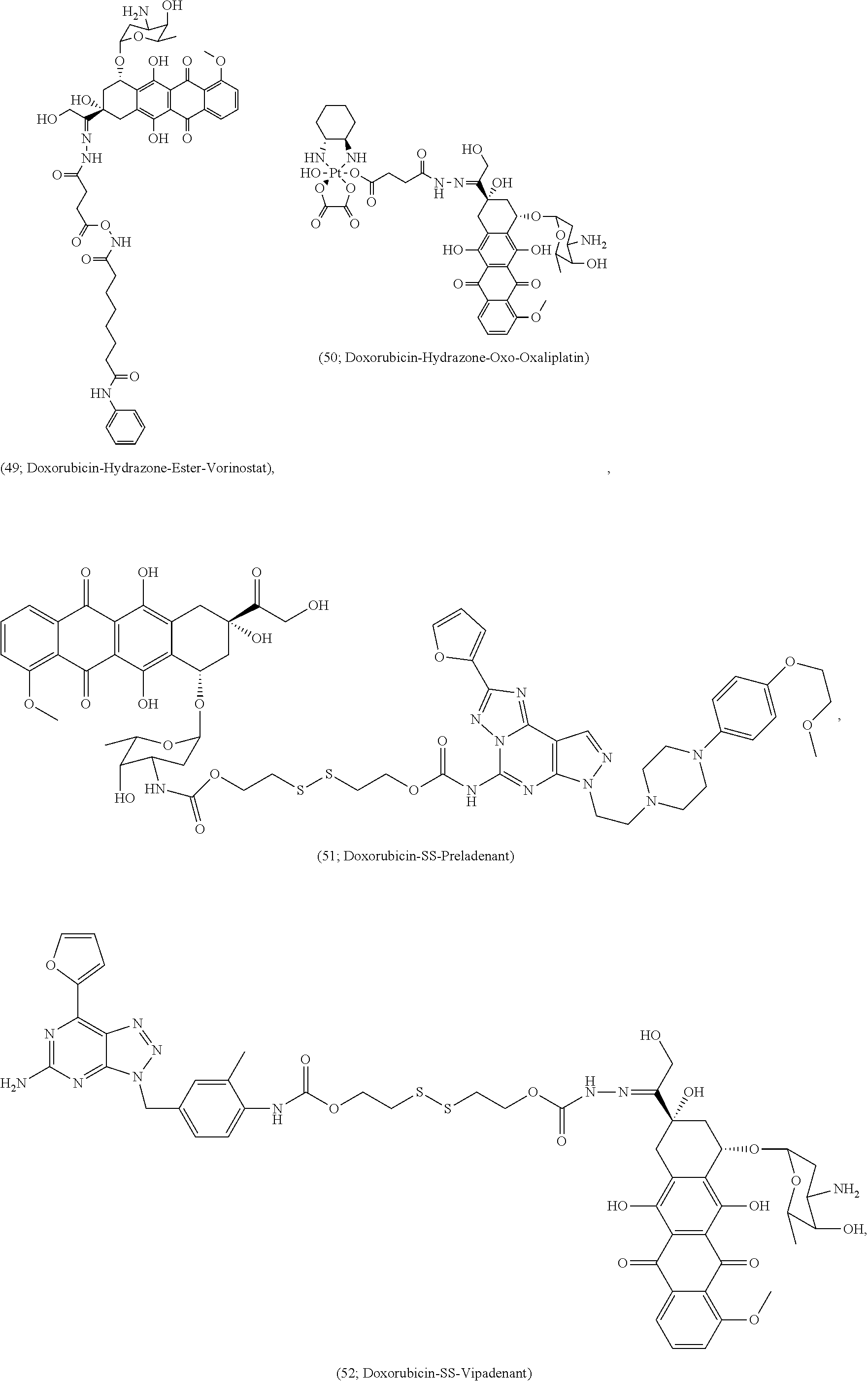

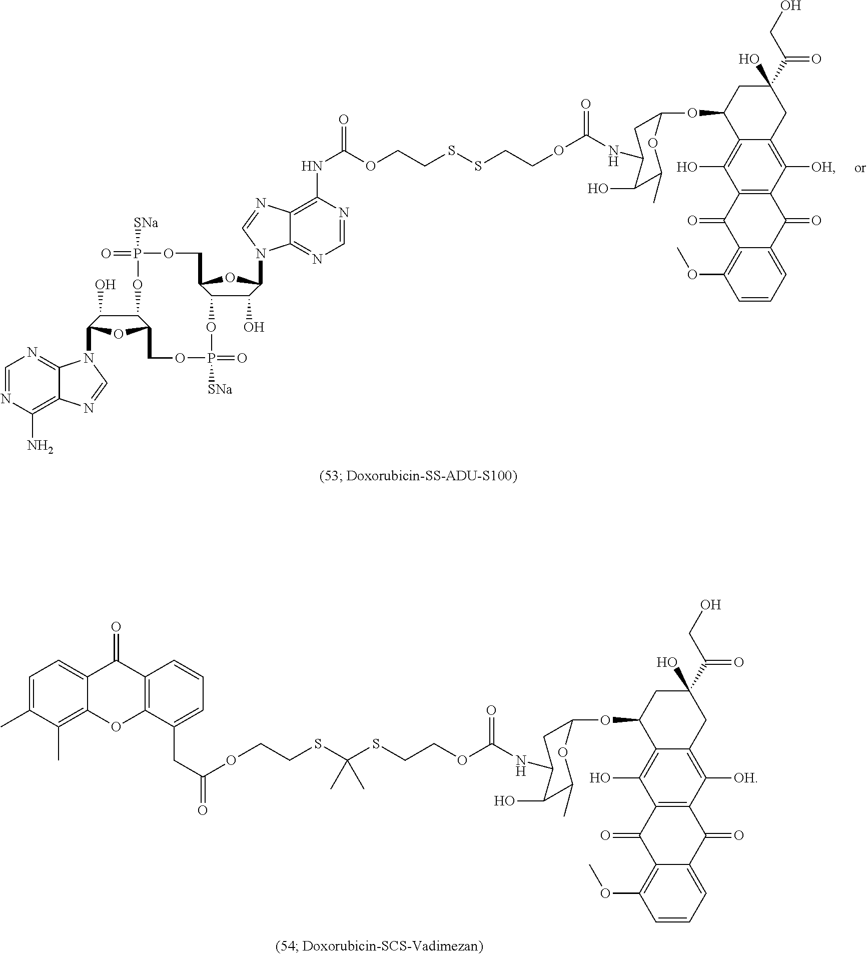

### BRIEF DESCRIPTION OF THE DRAWINGS

FIG. 1A-1G. Development of SM-derived camptothecin liposomal nanovesicles (Camptothesomes). FIG. 1A: Chemical structure of sphingomyelin (SM) and cam ptothecin (CPT), conjugation of SM and CPT to result in SM-derived CPT with ester bond (SM-Ester-CPT), with disulfide linkage (SM-SS-CPT), with glycine bond (SM-Glycine-CPT), and with disulfide linkage and longer linker (SM-CSS-CPT). FIG. 1B: Schematic depicting the self-assembling process of SM-CPT into Camptothesome. FIG. 1C: Cryogenic transmission electron microscopy (Cryo-EM) for Camptothesome-4. FIG. 1D: Dynamic light scattering (DLS) size distribution by intensity for Camptothesome-4. FIG. 1E: The fluorescence intensity for Camptothesome-4, SM-CSS-CPT, SM, cholesterol, and DSPE-PEG2K in methanol at equivalent (eq.) concentration. The significant fluorescence quenching for SM-CSS-CPT after self-assembling into LN demonstrate strong 7-7 stacking interactions among SM-CSS-CPT molecules since CPT contains several aromatic rings. FIG. 1F: % closed lactone in PBS (pH 8.4) as a function of time for 4 different SM-CPT conjugates. SM-conjugated CPTs dramatically prevented the lactone form from being converted into inactive carboxylate form, enhancing CPT stability. FIG. 1G: DLS size by intensity and zeta potential monitoring over time in 5% dextrose at 4° C. for 4 different Camptothesomes. Camptothesome-4 maintained its integrity for up to 2 months (FIG. 2). Data are expressed as mean±SD. #p<0.0001 (one-way ANOVA followed by Tukey's post-hoc test).

FIG. 2A-2B. The DLS size (FIG. 2A) and zeta potential (FIG. 2B) monitoring for Camptothesome-4 over a 60-day period after preparation.

FIG. 3A-3C. Fluorescence quenching of SM-Ester-CPT (FIG. 3A), SM-SS-CPT (FIG. 3B), and SM-Glycine-CPT upon self-assembling into Camptothesome (FIG. 3C).

FIG. 4A-4C. Development and physicochemical characterizations of Cy5.5-labeled-Camptothesome-4. The DLS size distribution by intensity for 0.1 weight % (FIG. 4A), 0.2 weight % (FIG. 4B), or 0.3 weight % (FIG. 4C) of DSPE-Cy5.5 in Cy5.5/Camptothesome-4.

FIG. 5A-5B. The representative CryoEM images (FIG. 5A) and DLS size by intensity (FIG. 5B) for Camptothesome-1, Camptothesome-2, and Camptothesome-3.

FIG. 6A-6F. Camptothesomes increased the maximum tolerated dose (MTD) of CPT without systemic toxicities in healthy mice. FIG. 6A: The mice weight change in MTD study of free CPT (formulated in 10% Tween 80/0.9% NaCl (9:1, v/v) with 20 m in sonication) [25] and 4 SM-CPTLNs at various doses as indicated in healthy BALB/c m ice following a single IV administration via tail vein (n=3); mice body weight and survival were monitored for 14 days. The MTD is defined by the dose that did not cause mouse death or more than 15% weight loss within the monitoring period [28, 30]. The mouse weight curve was terminated when there was the occurrence of mouse death. FIG. 6B-F: On day 14 post IV injection, blood samples were withdrawn for, leukocytes (FIG. 6B), erythrocytes (FIG. 6C), and thrombocytes (FIG. 6D) serum chemistry (FIG. 6E) analysis carried out by Arizona University Animal Care Pathology Services Core, and the heart, liver (blue arrow: hepatic steatosis; black arrow: diffuse microvesicular degeneration of hepatocytes) [30, 31], and kidney (yellow arrow: hemorrhage in interstitial tissue) [31, 32] were isolated for hematoxylin and eosin (H&E) FIG. 6F, staining by Tissue Acquisition and Cellular/Molecular Analysis Shared Resource at University of Arizona Cancer Center from the mice in MTD dose group for free CPT (5 mg/kg), Camptothesome-1 (120 mg CPT/kg), Camptothesome-2 (30 mg CPT/kg), Camptothesome-3 (80 mg CPT/kg), and Camptothesome-4 (30 mg CPT/kg), as well as vehicle control group (5% dextrose). Data are expressed as mean±SD. *p<0.05, **p<0.01, ^{#}p<0.0001 (one-way ANOVA followed by Tukey's post-hoc test).

FIG. 7A-7F. Improved circulation half-life and tumor delivery with efficient intratumoral drug release and deep tumor penetration. FIG. 7A: Blood kinetics of CPT in subcutaneous (SC) CT26 tumor bearing mice (n=3, ˜300 mm³) following IV injecting Camptothesomes (20 mg CPT/kg), and free CPT (5 mg/kg, MTD) once. FIG. 7B-C: Tissue distribution (FIG. 7B) and CPT intratumoral release (FIG. 7C) at 24 h from mice in (FIG. 7A). Percent injected dose in Camptothesomes represent the released CPT and SM-conjugated CPT. Drug content in plasma and major tissues were measured by HPLC. FIG. 7D: Lago optical imaging to study the real-time tumor delivery efficiency of DSPE-Cy5.5-labeled Camptothesome-4 in SC CT26 tumor bearing BALB/c mice (n=4, ˜300 mm³) at the indicated time points post an IV administration. FIG. 7E: Ex vivo imaging for visualization of free DSPE-Cy5.5 and Cy5.5/Camptothesome-4 distribution in different organs. FIG. 7F: Investigation of the ability of Camptothesome-4 to extravasate and penetrate the tumor after its being IV administered into mice with SC CT26 tumors (n=3, ˜300 mm³). 24 h after IV injection of Cy5.5/Camptothesome-4 (red), confocal laser scanning microscopy (CLSM) of sections of CT26 tumors were performed. Blood vessels were marked with PECAM-1 (platelet endothelial cell adhesion molecule) antibody followed by Alexa Fluor 488 secondary antibody staining (green); cell nucleus was stained by DAPI (blue). Scale bars: 50 μm. The results are expressed as mean±SD. *p<0.05, **p<0.01, ^{#}p<0.0001 compared to free CPT (one-way ANOVA followed by Tukey's post-hoc test).

FIG. 8A-8M. Camptothesome synergizes with PD-L1/PD-1 blockade to eradicate CRC tumors. FIG. 8A: Individual tumor growth curves (TGC) in SC CT26 tumor-bearing mice (n=6) IV injected once by free CPT (5 mg/kg), Camptothesomes and Onivyde® at 20 mg CPT or irinotecan/kg on day 9 (˜50 mm³); 5% dextrose was vehicle control; average TGC. FIG. 8B: Kaplan-Meier survival curves. FIG. 8C: Tumor IHC staining (PD-L1, PD-1, and IFN-γ) at day 7 post IV administering Camptothesome-4 (20 mg CPT/kg) one-time to CT26 tumor-bearing mice (n=3, ˜200 mm³). α-IFN-γ was intraperitoneally (IP) injected (200 μg/mouse/3 days) [37]. FIG. 8D: Individual TGC in CT26 tumor mice (n=5) IV administered once by Camptothesome-4 (30 mg CPT/kg), or combined with IP α-PD-L1 or α-PD-L1/α-PD-1 (100 μg/mouse/3 days, 3 times) [19] with or without α-IFN-γ on/from day 10. Mice images were taken on day 21; red circle shows tumor-free. FIG. 8E: Average TGC. FIG. 8F: Mice weight. FIG. 8G-8H: Tumors (FIG. 8D-8F) immune phenotypic analysis using IHC. FIG. 8I: Individual TGC in MC38 tumor-bearing mice (n=6, ˜50 mm³) IV administered with Camptothesome-4 (20 mg CPT/kg) once. α-PD-L1 and α-PD-1 were used similarly. FIG. 8J: Average TGC; FIG. 8K-M: Tumor-bearing mice images on day 26 (FIG. 8K, one death from vehicle control on day 23); Kaplan-Meier survival curves (FIG. 8L); 5 tumor-free mice from group fin (FIG. 8L) were re-challenged with MC38 cells on day 85 (FIG. 8M). Scale bar=100 μm (FIG. 8C, 8G). Data are mean±SD. *p<0.05, **p<0.01, ^{#}p<0.0001 (one-way ANOVA followed by Tukey's post-hoc test).

FIG. 9A-9C. Tissue distribution for Camptothesome-4. An independent biodistribution study was performed in SC CT26 tumor-bearing Balb/c mice (n=3) at 2.5 h (FIG. 9A) and 72 h (FIG. 9B) post IV administration of Camptothesome-4 (20 mg CPT/kg). FIG. 9C: Intratumoral release of CPT at 2.5 h and 7.2 h from mice (FIG. 9A-B).

FIG. 10A-10C. Tumor-bearing mice images taken on day 23 (FIG. 10A-B) and the mice body weight (FIG. 10C) from the antitumor efficacy study shown in FIG. 8A.

FIG. 11A-11E. Therapeutic efficacy of combing α-PD-1 and Camptothesome-4 in SC CT26 tumor murine model. Mice were SC inoculated with 1×10⁵ CT26 cells on day 0. On day 9 (˜50 mm³ tumors), mice (n=6) were IV administered once by 5% dextrose (vehicle control), free CPT (5 mg/kg, MTD) or Camptothesome-4 (20 mg CPT/kg, 2/3 MTD). α-PD-1 was IP injected (200 μg/mouse) from day 9 every 3 days for 3 times. FIG. 11A: Individual tumor growth curves. FIG. 11B: Average tumor growth curves. FIG. 11C: Mice body weight monitoring. FIG. 11D: Kaplan-Meier survival curves. FIG. 11E: Tumor-bearing mice images taken on day 21. Data are expressed as mean±SD. *p<0.05, ^{#}p<0.0001 (one-way ANOVA followed by Tukey's post hoc test; survival curves were analyzed by Log-rank Mantel-Cox test).

FIG. 12A-12B. IHC staining (FIG. 12A) and normalized intensity compared to vehicle control (5% dextrose) (FIG. 12B) for IFN-γ in CT26 tumors from FIG. 8D-E.

FIG. 13. Mice body weight in efficacy study presented in FIG. 8I-J.

FIG. 14A-14B. IHC staining (FIG. 14A) and quantitative analysis (FIG. 14B) for IDO1 in CT26 tumors in FIG. 8B-8C. FIG. 14C shows a schematic for IDO1 pathway entailing the downstream mTOR, GCN2, and AHR signaling.

FIG. 15A-15B. IHC staining (FIG. 15A) and quantitative analysis (FIG. 15B) for PD-L1, PD-1, and IFN-γ in MC38 tumors. SC MC38 tumor-bearing C57BU6 mice (˜200 mm³) received a single IV injection of Camptothesome-4 (20 mg CPT/kg) and vehicle control (n=3). α-IFN-γ was IP injected at 200 μg/mouse/3 days. At day 7 post treatment, tumors were collected and subject to IHC staining for PD-L1, PD-1, and IFN-γ.

FIG. 16A-160. Co-encapsulating DOX-IND into Camptothesome-4 using DOX as a transmembrane-enabling agent. FIG. 16A: Schematic for the synthesis of DOX-IND. FIG. 16B: Schematic of remotely incorporating IND into Camptothesome-4 utilizing DOX as a transmembrane-enabling agent using (NH₄)₂SO₄ as a concentration gradient. Once DOX-IND is inside Camptothesome-4, the acidic pH (˜5.3) produced by (NH₄)₂SO₄ breaks the hydrazone bond, releasing free DOX and IND intermediate. The —NH₂ group from both DOX and IND-SS—NH—NH₂ enables formation of (DOX-NH₃)₂SO₄ and (IND-SS—NH—NH₃)₂SO₄ aggregated salt, with dissociated SO₄²⁻, avoiding drug leakage/escaping. FIG. 16C Illustration of co-encapsulating DOX-IND into Camptothesome-4. FIG. 16D-16E: Size distribution (FIG. 16D) and Cryo-EM (FIG. 16E) of DOX-IND/Camptothesome-4. FIG. 16F-16K: Antitumor efficacy in SC MC38 tumor-bearing mice (n=6, ˜300 mm³) IV injected once on day 17 at eq. 20 mg CPT/kg and 6.7 mg DOX-IND/kg. α-CD8 was IP given (200 μg/mouse/3 days) from day 17 [40]. Average TGC (FIG. 16F); Western blotting for P-S6K (FIG. 16G) and RT-PCR for IL-6 (FIG. 16H); Mice images on day 23 (FIG. 161, one death from vehicle group on day 22) and IHC analysis (FIG. 16J-16K, scale bar=100 μm) in tumors. FIG. 16L-16M: A single dose was IV injected to SC MC38tumor-bearing mice (n=5, ˜400 mm³) on day 20 at eq. 15 mg CPT/kg and 5 mg DOX-IND/kg. α-PD-L1, α-PD-1, and α-IFN-γ were IP administered as described above. Individual TGC (FIG. 16L), average TGC (FIG. 16M), mice image on day 22 (FIG. 16N, one death from vehicle control on day 21), and survival curves (FIG. 16O). Data are mean±SD. *p<0.05, **p<0.01, ^{#}p<0.0001 (one-way ANOVA followed by Tukey's post-hoc test).

FIG. 17A-17B. CPT fluorescence quenching (FIG. 17A) and DOX-IND fluorescence quenching (FIG. 17B) in Camptothesome-4 and DOX-IND/Camptothesome-4.

FIG. 18A-18C. Development of DOX-IND-laden Camptothesome-4 with or with folate targeting. FIG. 18A: DOX release kinetics from DOX-IND inside LN after remote loading procedure. FIG. 18B: The size by dynamic light scattering (DLS). FIG. 18C: Cryo-EM.

FIG. 19. MTD Investigation for DOX-IND/Camptothesome-4 (2% of DOX-IND DLC).

FIG. 20A-20D. Individual tumor growth curves (FIG. 20A), mice body weight (FIG. 20B), and IHC staining for cleaved caspase-3, perforin and granzyme-B (FIG. 20C and FIG. 20D) from the therapeutic efficacy study presented in FIGS. 16E, 16H.

FIG. 21. Mice body weight change over time from the anticancer efficacy investigation displayed in FIG. 16K-16M.

FIG. 22A-22W. Eradication of advanced and metastatic orthotopic CRC and melanoma tumors. FIG. 22A-22K: Therapeutic efficacy, and antitumor immunity in orthotopic CRC tumor mouse model. Mice were inoculated with 2×10⁶ CT26-Luc cells (DMEM/Matrigel, 3/1, v/v) into the cecum subserosa [41, 42]. On day 8, mice (n=5, ˜300 mg) were IV administered once with Camptothesome-4, DOX-IND/Camptothesome-4, or Folate/DOX-IND/Camptothesome-4 at eq. 15 mg CPT/kg and 5 mg DOX-IND/kg. α-PD-L1 and α-PD-1 were injected as described above. Lago imaging for live mice with orthotopic CRC tumors (FIG. 22A-22F). Red circle means tumor-free (one mouse from vehicle control and α-PD-L1+α-PD-1 groups died on day 18). Quantitative bioluminescence intensity (QBI) for whole mice tumor burden (FIG. 22G). QBI (FIG. 22H) and a heatmap summarizing tumor metastatic rate (FIG. 22I), and representative ex vivo photograph (upper panel) and bioluminescence imaging (FIG. 22J, lower panel) in various organs on day 18. Immune phenotypic analysis of tumor tissues using IHC (FIG. 22K-22N). FIG. 220-22W: Antitumor efficacy in melanoma-bearing C57BL/6 mice. Animals were SC inoculated with 0.1×10⁶ B16-F10-Luc2 cells [43]. On day 14, mice (n=5, ˜400 mm³) received same treatments in (FIG. 22A-22F). Live mice Lago imaging (FIG. 220-22T). Two mice from vehicle control died on day 20. QBI for whole mice tumor burden (FIG. 22U). QBI (FIG. 22V), a heatmap presenting the tumor metastatic rate (FIG. 22W). Data are expressed as mean±SD. *p<0.05, **p<0.01, ^{#}p<0.0001 (one-way ANOVA followed by Tukey's post-hoc test).

FIG. 23. Representative ex vivo Lago bioluminescence imaging (left panel) and photographs (right panel) for various tissues in orthotopic CRC murine model on day 8 post injecting 2×10⁶ CT26-Luc cells into the cecum subserosal.

FIG. 24A-24C. Pharmacokinetics and biodistribution in orthotopic CRC murine model. 2×10⁶ CT26-Luc cells were injected in the cecum subserosa of Balb/c mice. 8 days later, mice were IV administered once with Doxil, free CPT, DOX-IND/Camptothesome-4, or Folate/DOX-IND/Camptothesome-4 at eq. 20 CPT/kg, 1.7 mg IND/kg or 4 mg DOX/kg. Blood kinetics and tissue distribution of CPT (FIG. 24A), DOX (FIG. 24B) and IND (FIG. 24C). Data are expressed as mean±SD. *p<0.05, **p<0.01, #p<0.0001 (one-way ANOVA followed by Tukey's HSD multiple comparison post hoc test).

FIG. 25A-25D. Mice body weight (FIG. 25A) and IHC staining for PD-L1, PD-1, and IDO1 (FIG. 25B), Foxp3, IFN-γ, granzyme B, IL-10 and IL-12 (FIG. 25C-D) from efficacy study shown in FIG. 22A-22G.

FIG. 26A-26F. Tumor weight in response to treatment (FIG. 26A) and IHC for PD-L1, PD-1, and IDO1 (FIG. 26B); CD8, Foxp3, Calreticulin, IFN-γ, Granzyme B, Perforin, and LRP1 (FIG. 26C-26E); representative ex vivo bioluminescence imaging in various organs (FIG. 26F) in orthotopic melanoma tumors from therapeutic efficacy study in FIG. 220-22U.

FIG. 27 is a representative schematic for the synthesis of SM-Ester-EPA in accordance with embodiments of the present disclosure.

FIG. 28A-28D. Physicochemical characterizations of the self-assembled nanovesicles formed from the SM-Ester-EPA of FIG. 27 at five different lipids molar ratios (FIG. 28A); representative DLS size distribution by intensity (FIG. 28B); the DLS size monitoring of SM-EPA nanovesicles over time (FIG. 28C); the zeta potential monitoring of SM-EPA nanovesicles over time (FIG. 28D);

FIG. 29 shows the IDO1 inhibition rate in Hela cells of the SM-Ester-EPA nanovesicles of FIG. 27 as compared to free EPA at equivalent EPA concentration.

FIG. 30A-30C shows the studies of pharmacokinetics (FIG. 30A), tissue biodistribution (FIG. 30B), and intratumoral drug release (FIG. 30C) in B16-F10 melanoma m ice intravenously injected once by the SM-Ester-EPA nanovesicles of FIG. 27 as compared to free EPA at equivalent 10 mg EPA/kg.

FIG. 31A-31C. Tumor growth curves (FIG. 31A), mice survival curves (FIG. 30B), and mice image taken on day 15 in B16-F10 melanoma-bearing mice treated by SM-EPA nanovesicles of FIG. 27, SM-EPA nanovesicles of FIG. 27 plus α-PD-1, α-PD-1, free EPA, or free EPA plus α-PD-1. EM-EPA nanovesicles and free EPA were intravenously administered at equivalent 41 mg EPA/kg on day 8, 10, 12, and 14. α-PD-1 was intraperitoneally injected at 100 ug/mouse on day 8, 11, and 14.

FIG. 32 are representative graphs showing the IFN-γ⁺/CD8⁺ T cells, Granzyme-B (Gr-B)⁺/CD8⁺ T cells, or Foxp3⁺/CD25⁺ T cells in tumors on day 15 from the B16-F10 melanoma-bearing mice treated the same as described in FIG. 31 in an independent assay.

### DETAILED DESCRIPTION

As set forth in the experimental examples herein, sphingomyelin-drug conjugates described herein can from nanovesicles, which can then be loaded with other anti-cancer drug conjugates resulting in a co-delivery platform that offers several advantages over existing ICB therapies. Specifically, sphingomyelin-drug conjugates have been synthesized and formulated into nanovesicles, comprising one or more anti-cancer drugs, where the one or more anti-cancer drugs (e.g., hydrophobic or hydrophilic drugs) are incorporated into a lipid bilayer of the nanovesicles. The nanovesicles can then be loaded with additional drugs (e.g., hydrophilic or hydrophobic drugs) by incubating the nanovesicles with doxorubicin (DOX)-drug conjugates described herein, where the DOX-drug conjugates cross the lipid bilayer of the nanovesicles, incorporating the additional drug into the interior core of the nanovesicles. The result is a therapeutic platform that enables the co-delivery of multiple drugs with different polarity and chemical structures (e.g., hydrophobic and hydrophilic drugs). The co-delivery of multiple drugs results in a synergistic effect thereby, providing a therapeutic platform with remarkable antitumor efficacy, where this co-encapsulation has not been achieved in existing liposomal platforms.

Based on this disclosure, provided herein are sphingomyelin-drug conjugates, nanovesicles formed from the sphingomyelin-drug conjugates and/or DOX-drug conjugates, methods of preparing these sphingomyelin-drug conjugates, DOX-drug conjugates, and nanovesicles, kits comprising these sphingomyelin-drug conjugates, DOX-drug conjugates, and nanovesicles, and methods of using these sphingomyelin-drug conjugates, DOX-drug conjugates, and nanovesicles in the treatment and/or prevention of cancer.

### Definitions

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present document, including definitions, will control. Preferred methods and materials are described below, although methods and materials similar or equivalent to those described herein can be used in practice or testing of the present invention.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

The terms “treat,” “treating,” and “treatment” as used herein with regard to a condition refer to alleviating the condition partially or entirely; slowing the progression or development of the condition; eliminating, reducing, or slowing the development of one or more symptoms associated with the condition; or increasing progression-free or overall survival of the condition.

The terms “prevent,” “preventing,” and “prevention” as used herein with regard to a condition refers to averting the onset of the condition or decreasing the likelihood of occurrence or recurrence of the condition, including in a subject that may be predisposed to the condition but has not yet been diagnosed as having the condition.

The term “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

As used herein, the terms “effective amount” or “therapeutically effective amount,” refers to an amount of the compound described herein that will elicit the biological or medical response of a subject, for example, reduction or inhibition of an enzyme or a protein activity, or ameliorate symptoms, alleviate conditions, slow or delay disease progression, or prevent a disease, etc. In one aspect, “therapeutically effective amount” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

“Liposome”, “nanovesicle” and “liposome vesicle” refers to a structure having a lipid-containing membrane (e.g., comprised of sphingomyelin-drug conjugated described herein) enclosing an interior core.

Definitions of specific functional groups and chemical terms are described in more detail herein. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

Certain compounds described herein may exist in particular geometric or stereoisomeric forms. A particular enantiomer of a compound described herein may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Unless otherwise stated, structures depicted herein are also meant to include geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure. Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the disclosure.

Thus, a composition containing 90% of one enantiomer and 10% of the other enantiomer is said to have an enantiomeric excess of 80%. The compounds or compositions described herein may contain an enantiomeric excess of at least 50%, 75%, 90%, 95%, or 99% of one form of the compound, e.g., the S-enantiomer. In other words, such compounds or compositions contain an enantiomeric excess of the S enantiomer over the R enantiomer.

Where a particular enantiomer is preferred, it may, in some embodiments be provided substantially free of the corresponding enantiomer and may also be referred to as “optically enriched.” “Optically enriched,” as used herein, means that the compound is made up of a significantly greater proportion of one enantiomer. In certain embodiments, the compound is made up of at least about 90% by weight of a preferred enantiomer. In other embodiments, the compound is made up of at least about 95%, 98%, or 99% by weight of a preferred enantiomer. Preferred enantiomers may be isolated from racemic mixtures by any method known to those skilled in the art, including chiral high-pressure liquid chromatography (HPLC) and the formation and crystallization of chiral salts or prepared by asymmetric syntheses. See e.g., Jacques et al., Enantiomers, Racemates and Resolutions (Wiley Interscience, New York, 1981); Wlen, et al., Tetrahedron 33:2725 (1977); Eliel, E. L. Stereochemistry of Carbon Compounds (McGraw Hill, N Y, 1962); Wlen, S. H. Tables of Resolving Agents and Optical Resolutions p. 268 (E. L. Eliel, Ed., Univ. of Notre Dame Press, Notre Dame, IN 1972).

All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed.

Any resulting mixtures of isomers can be separated based on the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds described herein into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Various exemplary embodiments of the disclosure are described herein. It will be recognized that features specified in each embodiment may be combined, substituted, or replaced with other specified features disclosed elsewhere in the specification to provide further embodiments of the present disclosure. All analagous compounds may be substituted for each other in the same or similar amounts (mass, concentration, or dosages) as indicated for analagous compounds.

It is understood that in the following embodiments, combinations of substituents or variables of the depicted formulae are permissible only if such combinations result in stable compounds.

### Sphingomyelin Drug Conjugates

Provided herein in certain embodiments, are sphingomyelin (SM) drug conjugates comprising sphingomyelin, a linker moiety, and an anti-cancer drug. Sphingomyelin comprises a hydroxyl (—OH)functional group, which allows conjugation via a linker moiety to drugs with functional group moieties such as —COOH, —OH, —NH₂ and C═O.

In some embodiments, the sphingomyelin drug conjugates of the present disclosure comprise a structure consistent with formula (I):

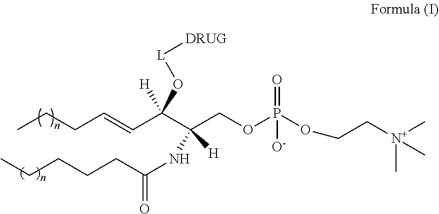

wherein:
n is independently 5 to 20;
L is a linker moiety; and
Drug is an anti-cancer drug.

In some embodiments, the sphingomyelin drug conjugates of the present disclosure comprise a structure consistent with formula (II):

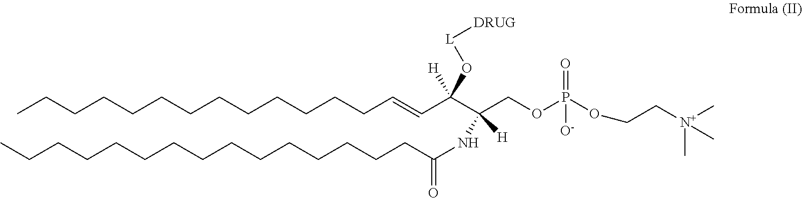

### Wherein:

L is a linker moiety; and
Drug is an anti-cancer drug.

In some embodiments, the linker moiety (L) of formulas (I)-(II) is selected from the group consisting of:

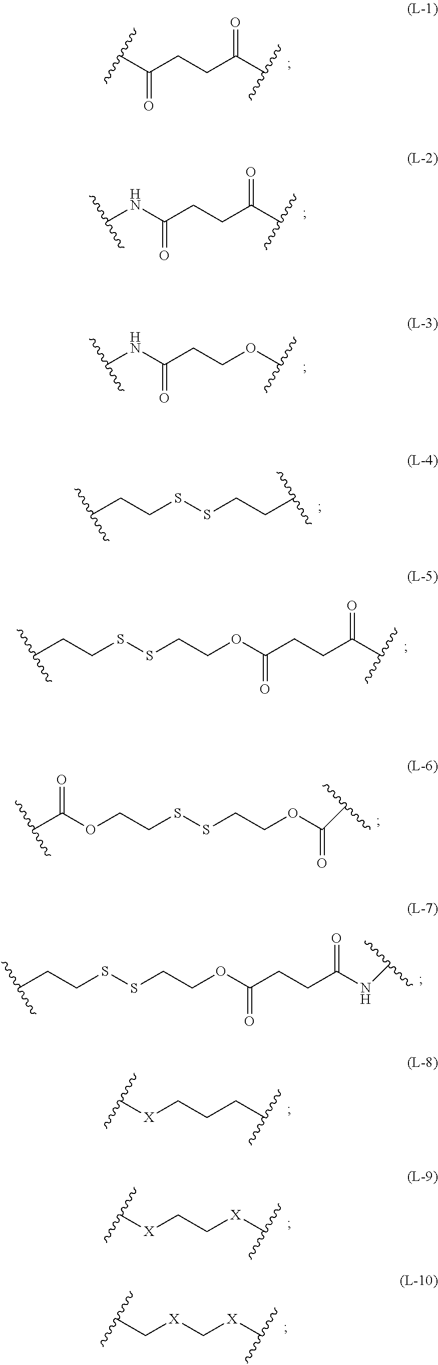

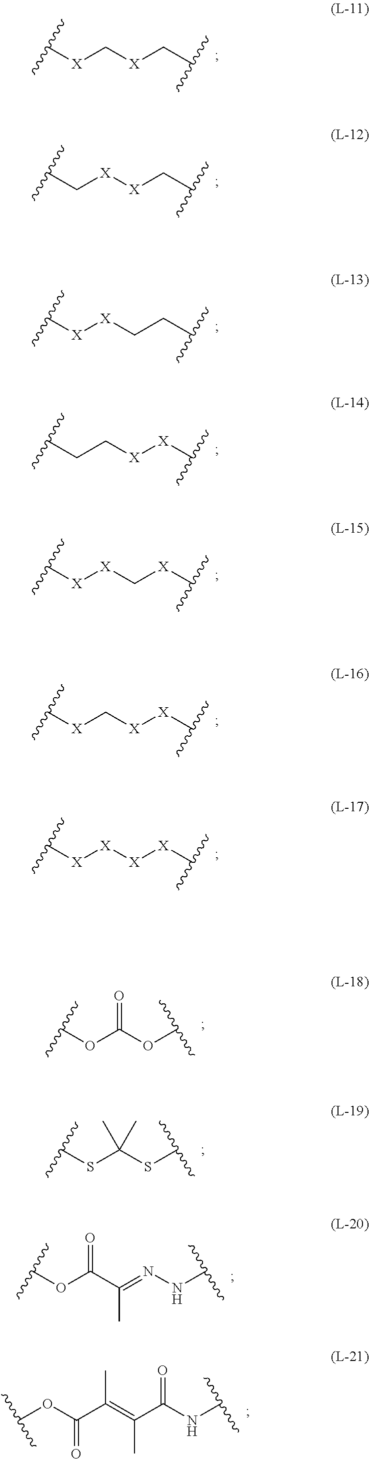

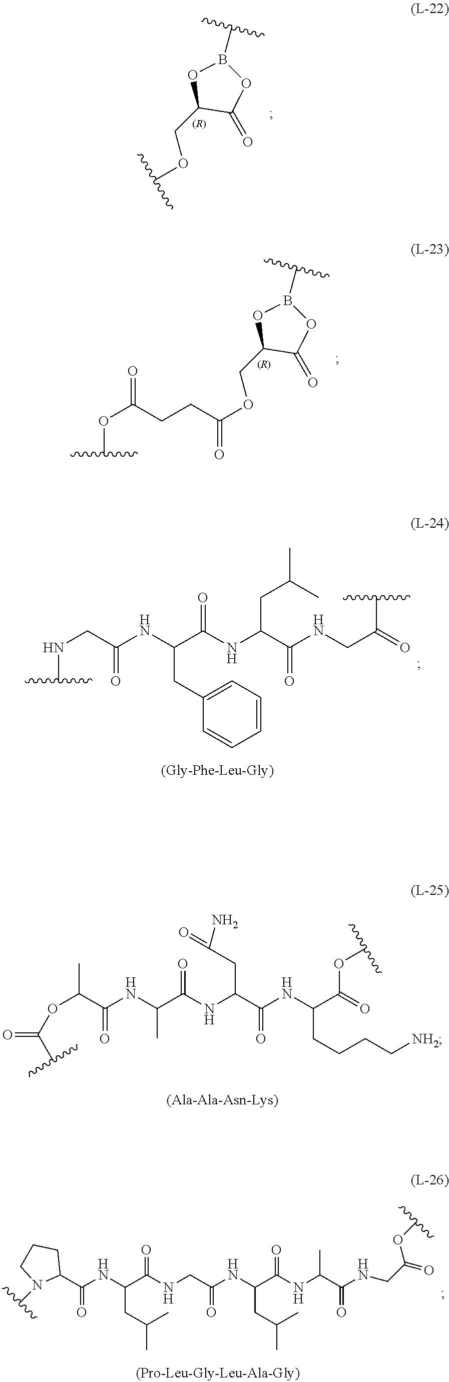

and combinations thereof,
wherein:
X is independently, O, S, —NH, or —CO.

In one embodiment, the anti-cancer drug comprises a functional group moiety that permits conjugation to sphingomyelin via the linker moiety. In some embodiments, the functional group moiety is selected from —COOH, —OH, —NH₂ and/or C═O. In some embodiments, the anti-cancer drug is a hydrophobic or hydrophilic drug.

Non-limiting examples of suitable anti-cancer drugs include cam ptothecin, paclitaxel, docetaxel, ADU-S100, amrubicin, 5-aminolevulinic acid, AZD4635, BMS-1001, BMS-1166, BMS-200, BMS-202, BMS-242, BMS-242, bortezomib, CA170, cabazitaxel, cabozantinib, canertinib, capecitabine, carboplatin, ceritinib, chlorin e6, cisplatin, dabrafenib, dacarbazine, darolutamide, daunorubicin, degarelix, digoxin, doxorubicin, epacadostat, epirubicin, eribulin, esorubicin, etoposide, fingolimod, 5-fluorouracil, galanthamine, gemcitabine, idarubicin, imatinib, imiquimod, indoximod, irinotecan, ixabepilone, lenvatinib, memantine, methotrexate, mitoxantrone, NIR178, NLG919, oxaliplatin, pazopanib, pemetrexed, preladenant, protoporphyrin IX (PP IX), pyropheophorbide-A, septacidin, SN-38, sorafenib, streptozocin, sunitinib, temozolomide, tipiracil, TPI-287, trifluridine, vadimezan, vemurafenib, vinblastine, vincristine, vinorelbine, vipadenant, vorinostat, or combinations thereof.

In some embodiments, sphingomyelin drug conjugates of the present disclosure comprise a chemical formula consistent with one or more of the following structures:

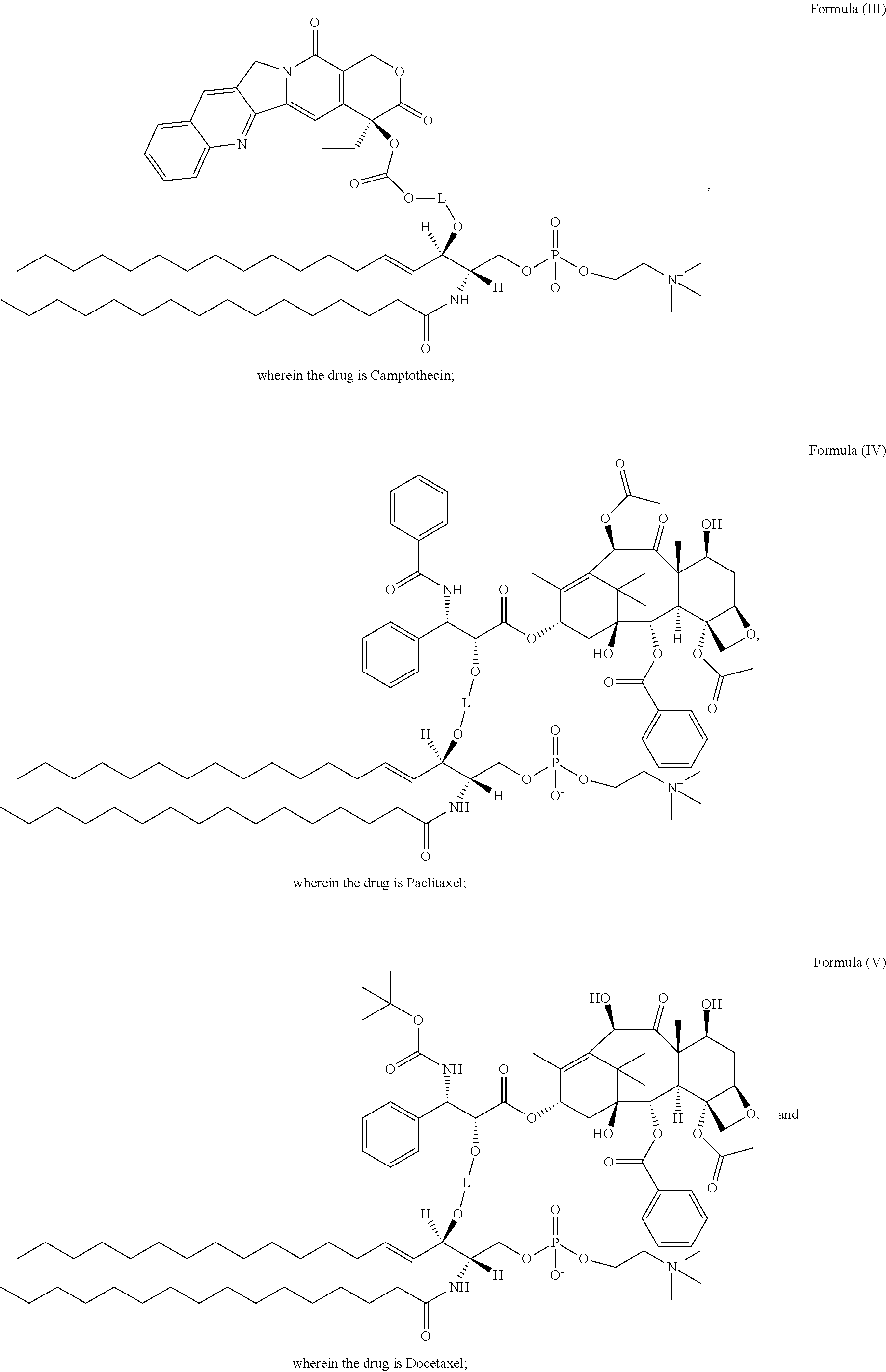

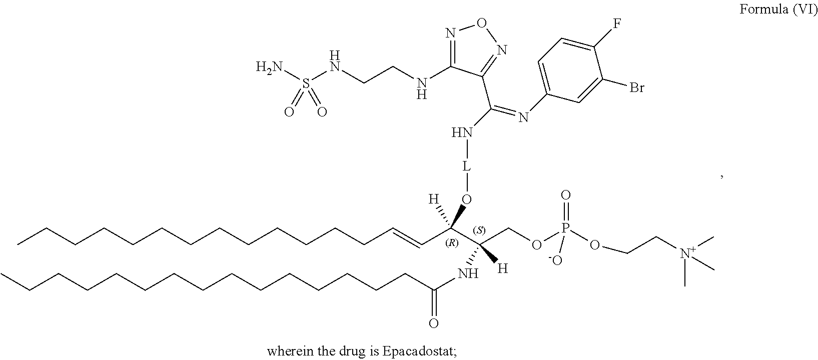

wherein L is a linker moiety.

In some embodiments, the sphingomyelin (SM) drug conjugates comprise a chemical formula consistent with one or more of the following chemical structures:

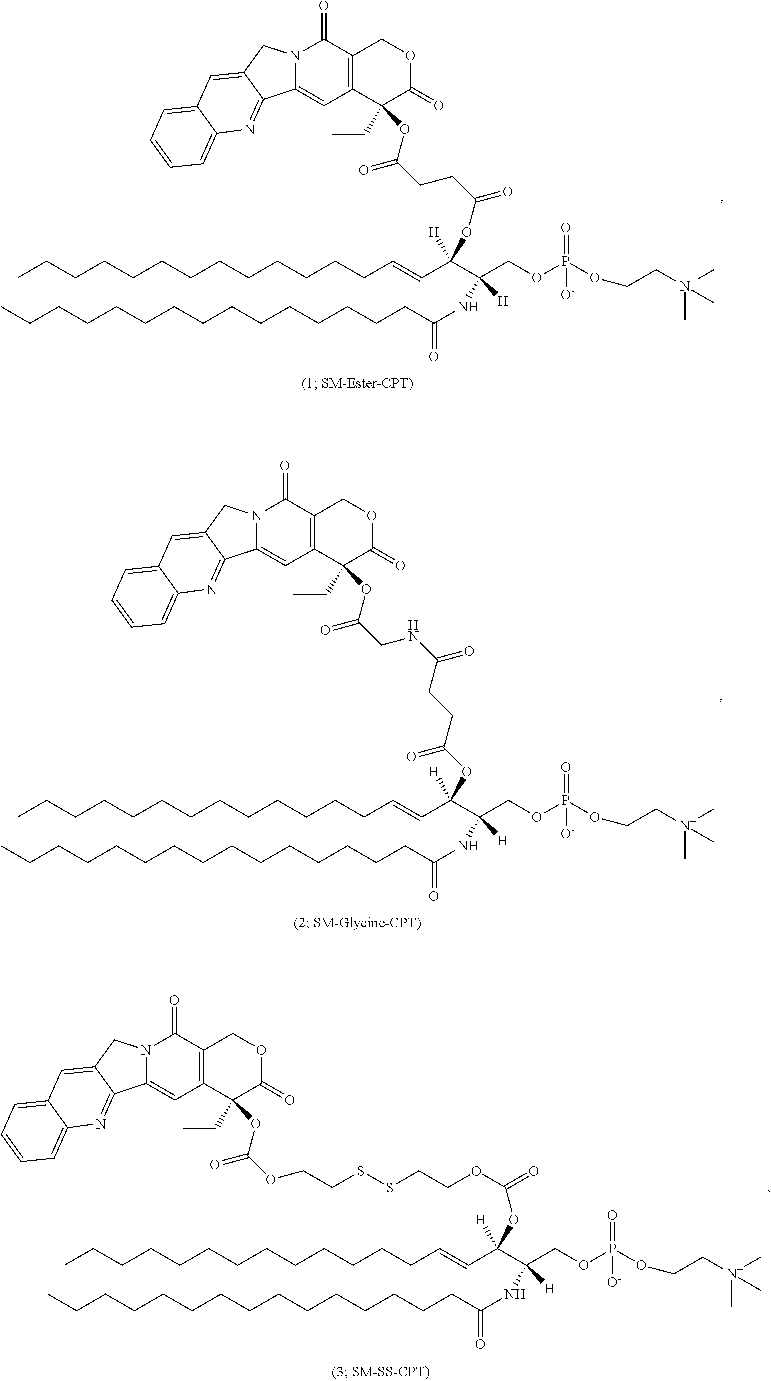

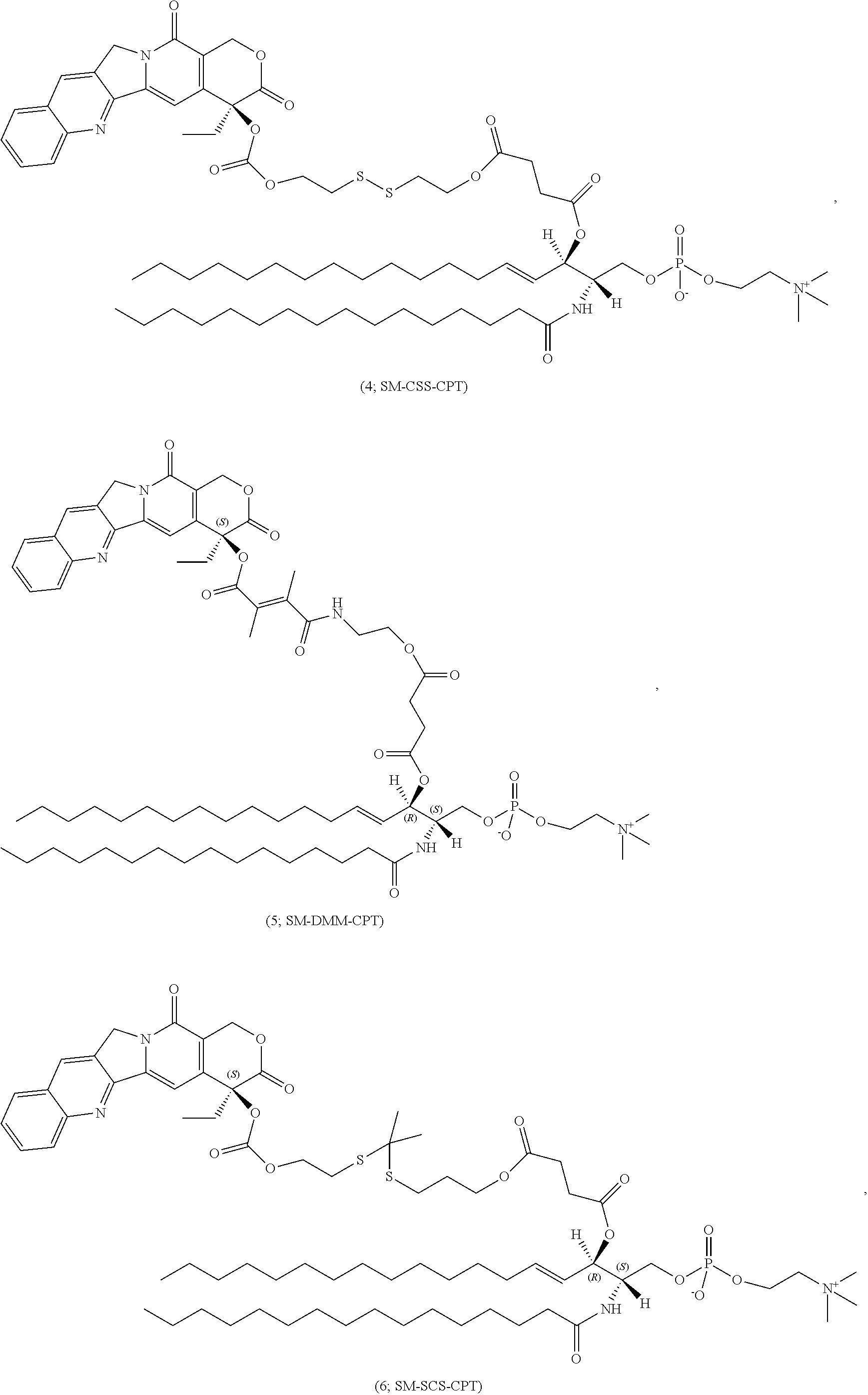

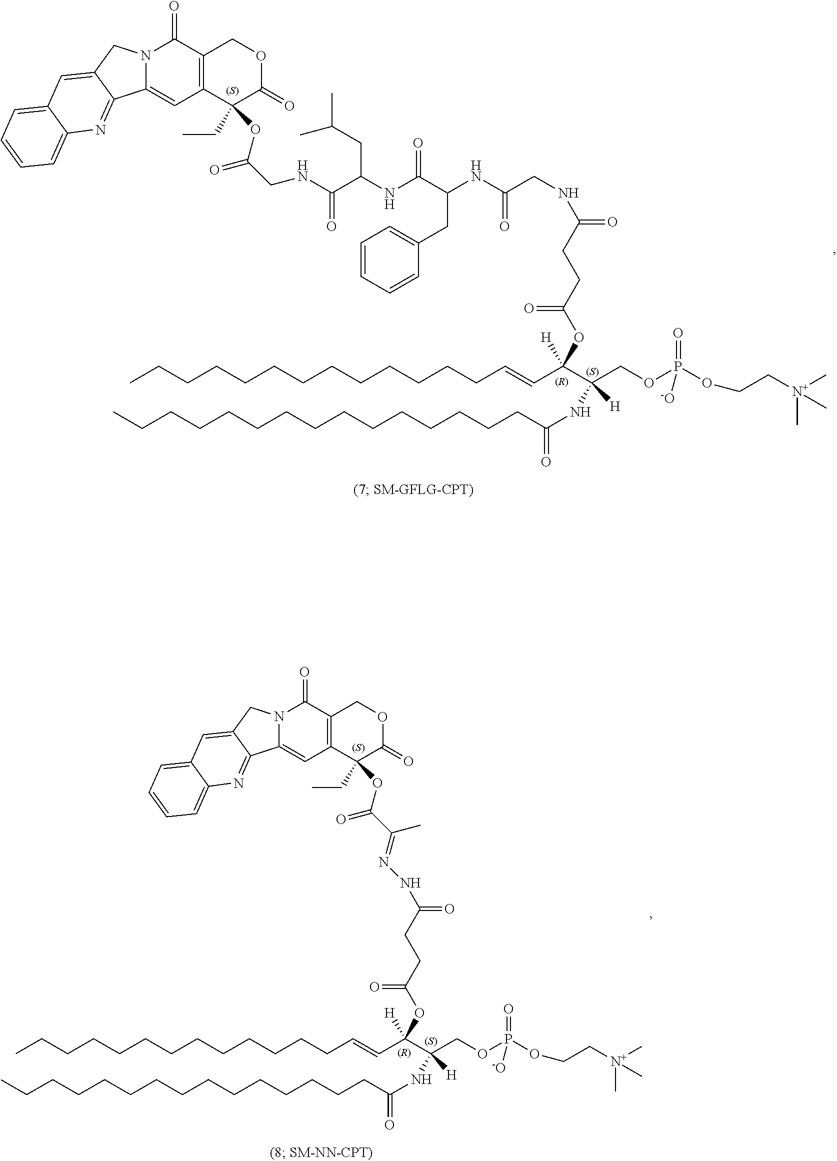

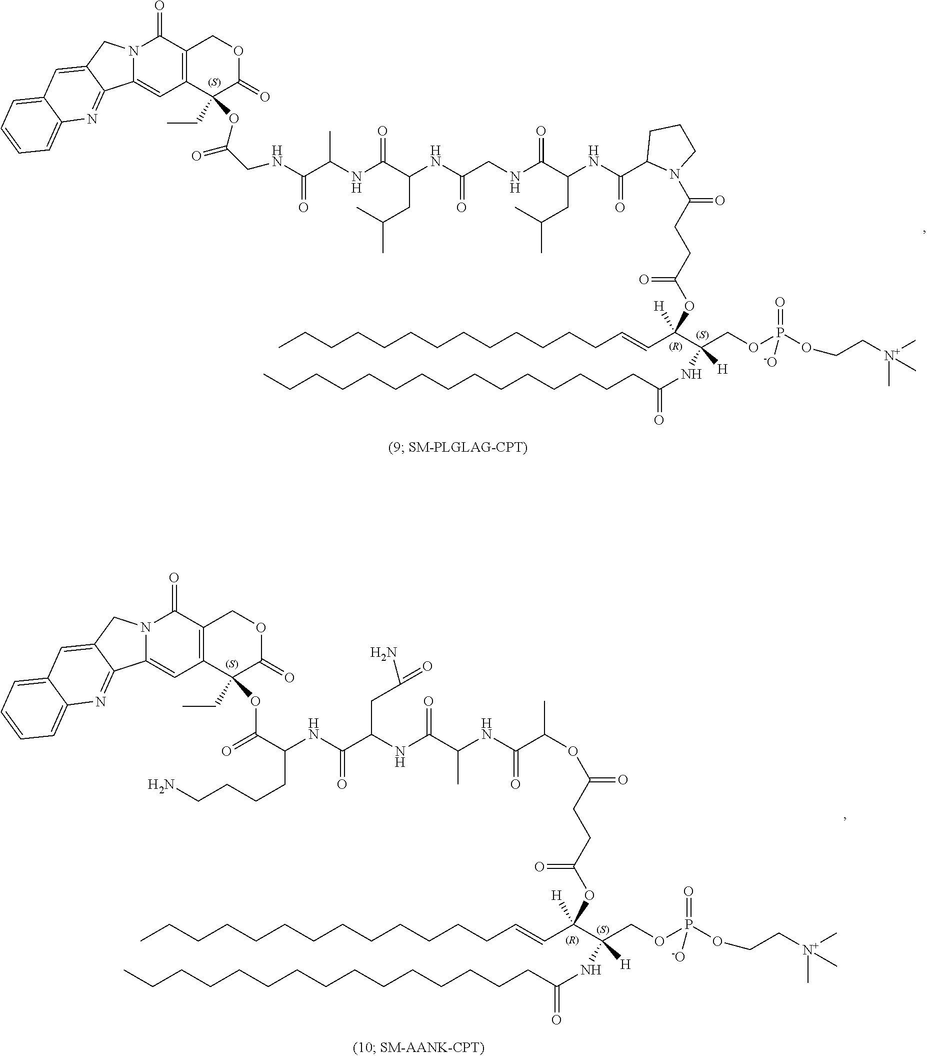

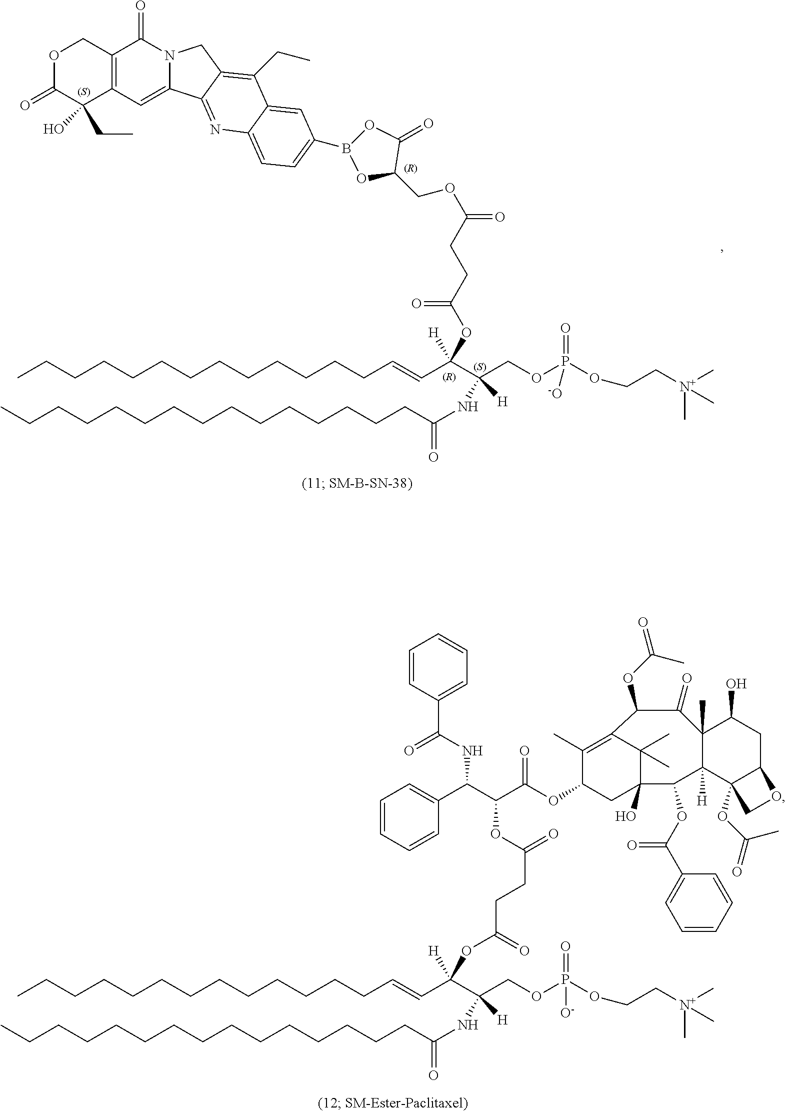

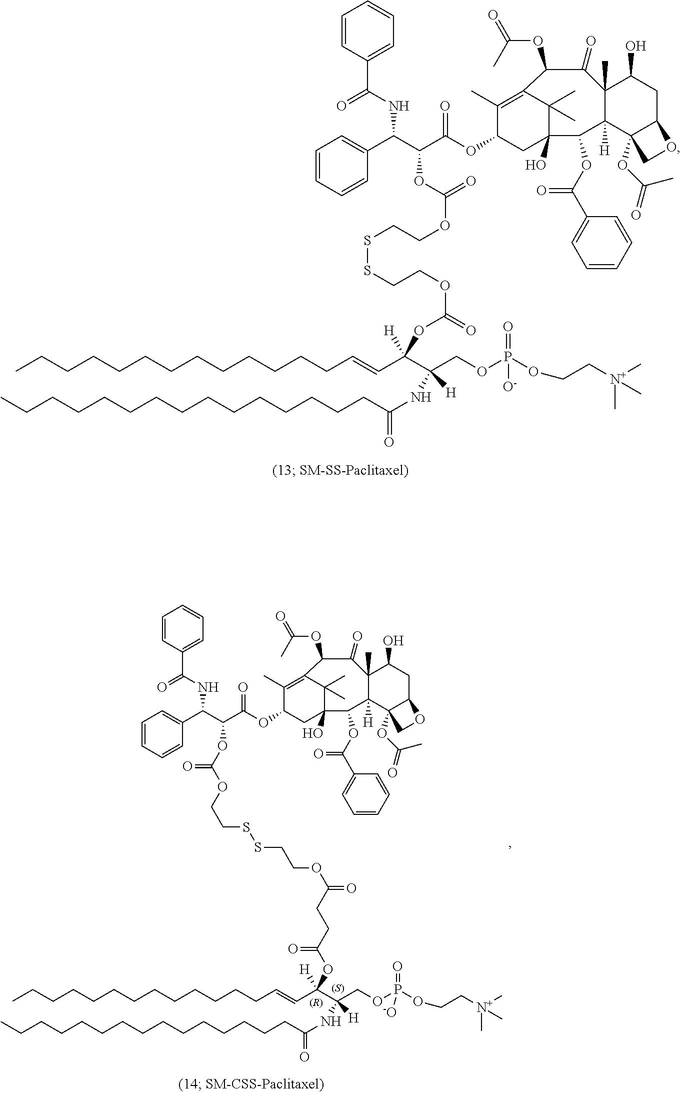

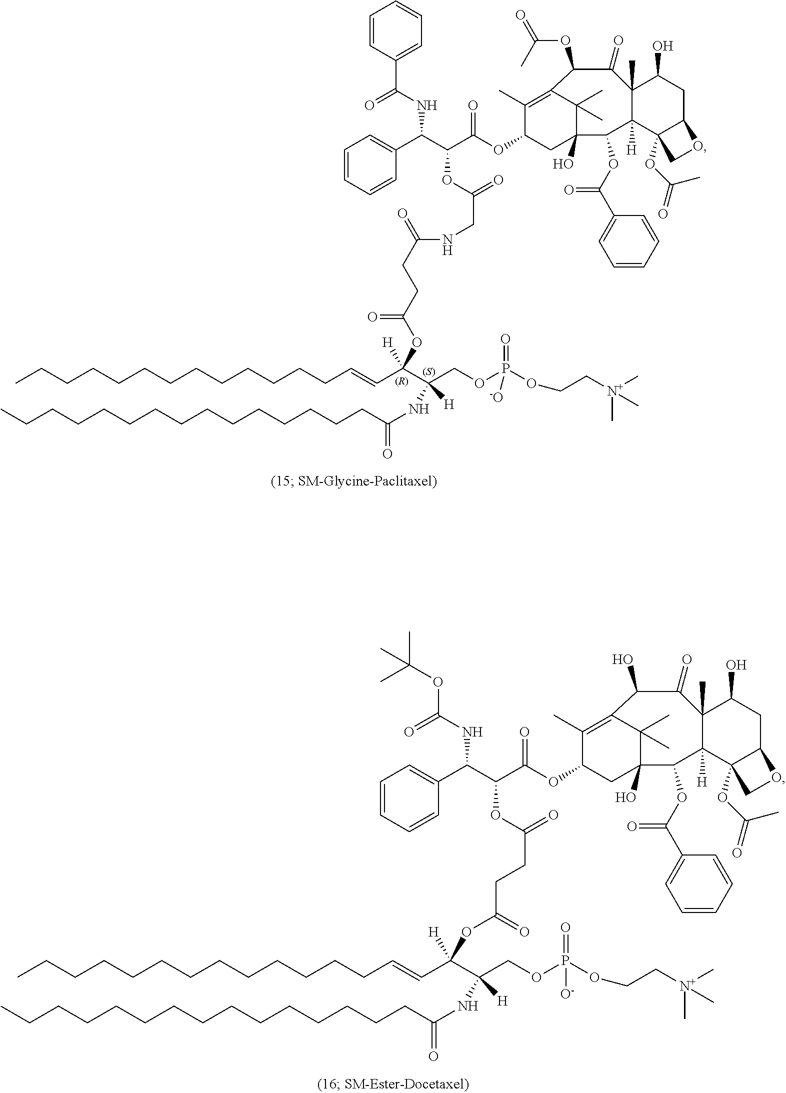

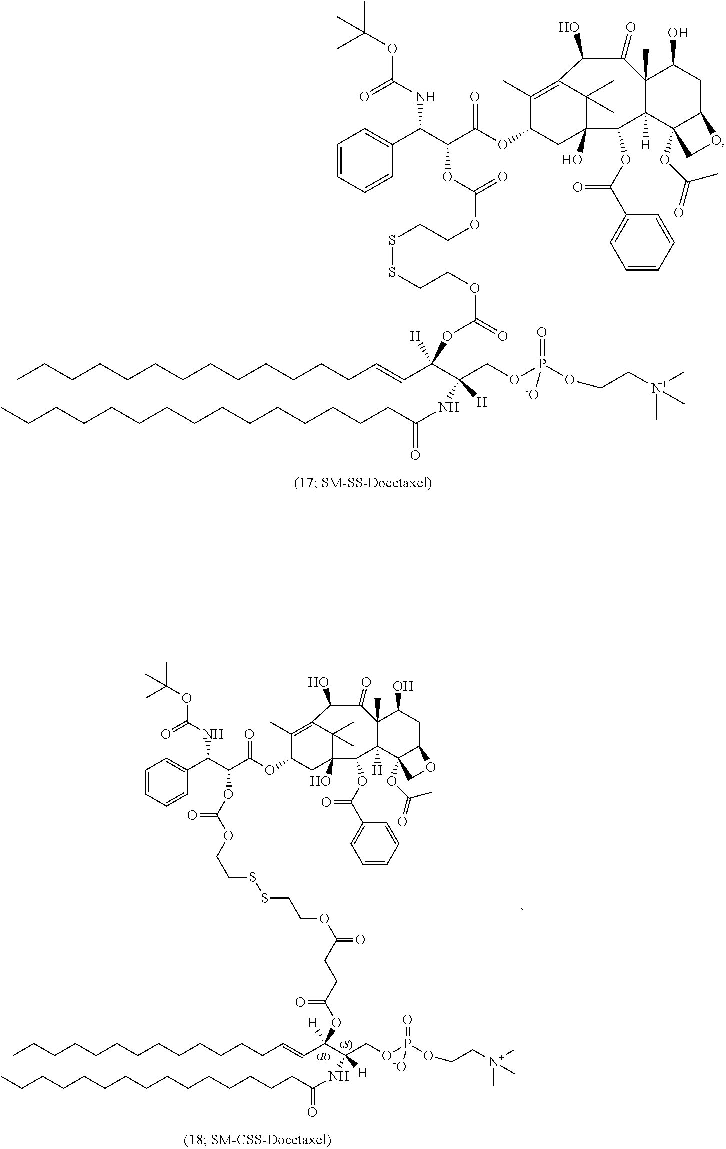

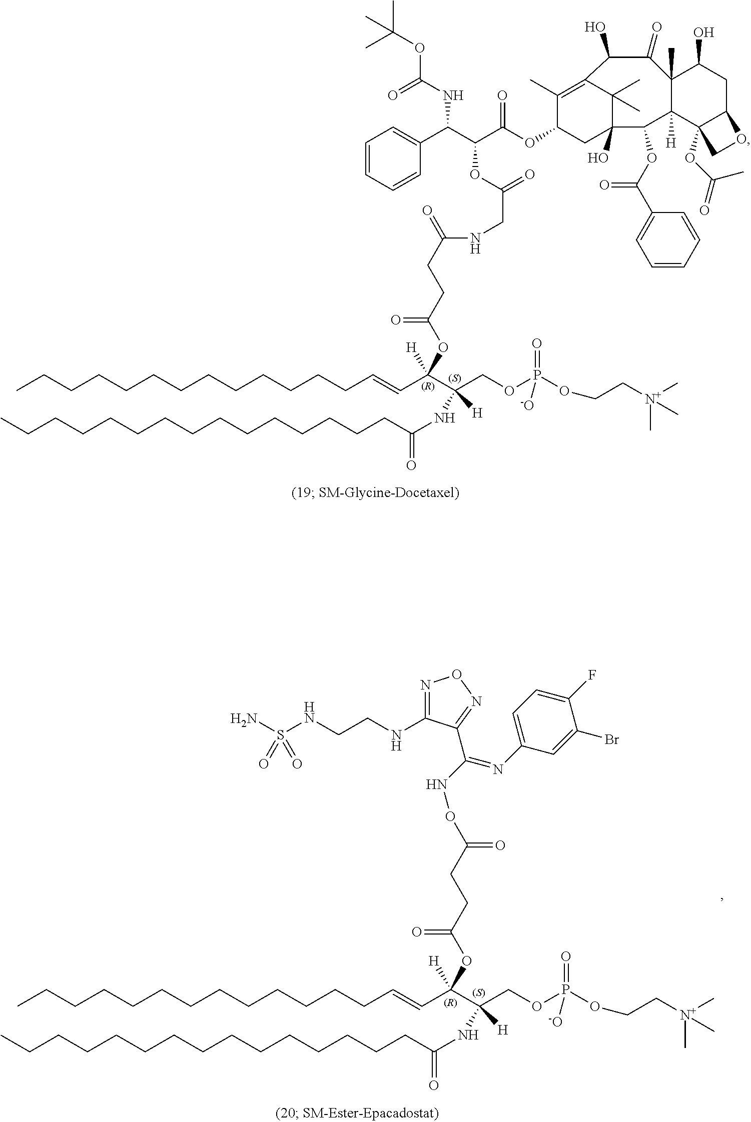

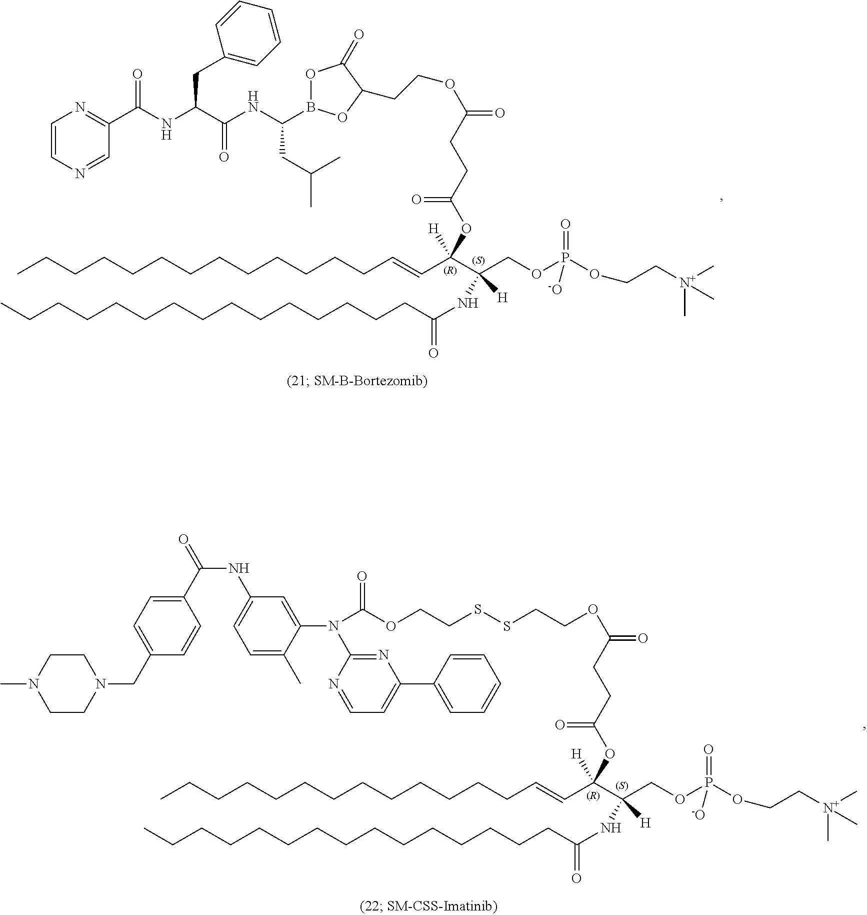

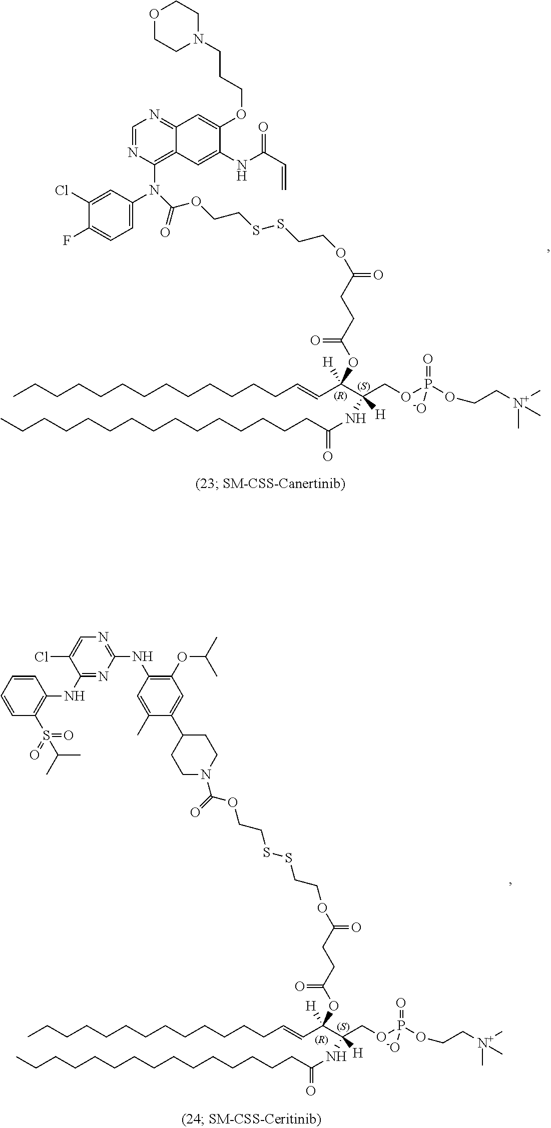

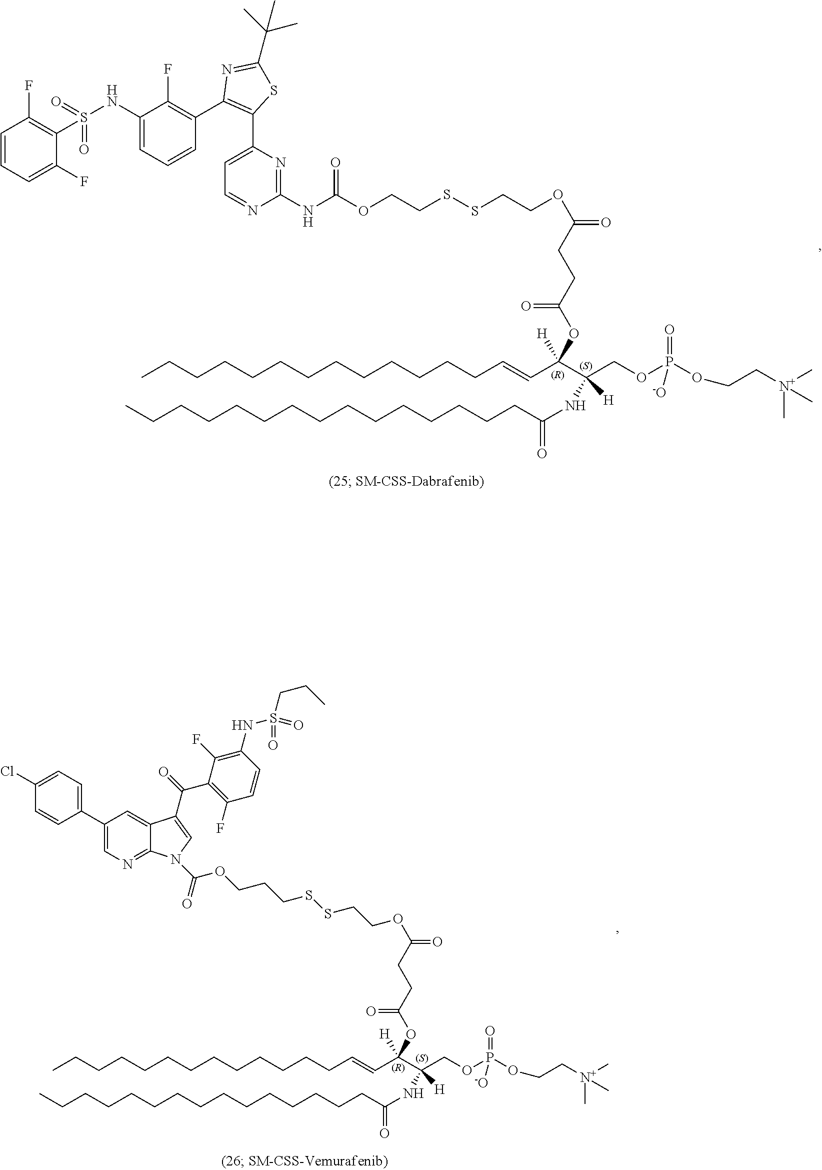

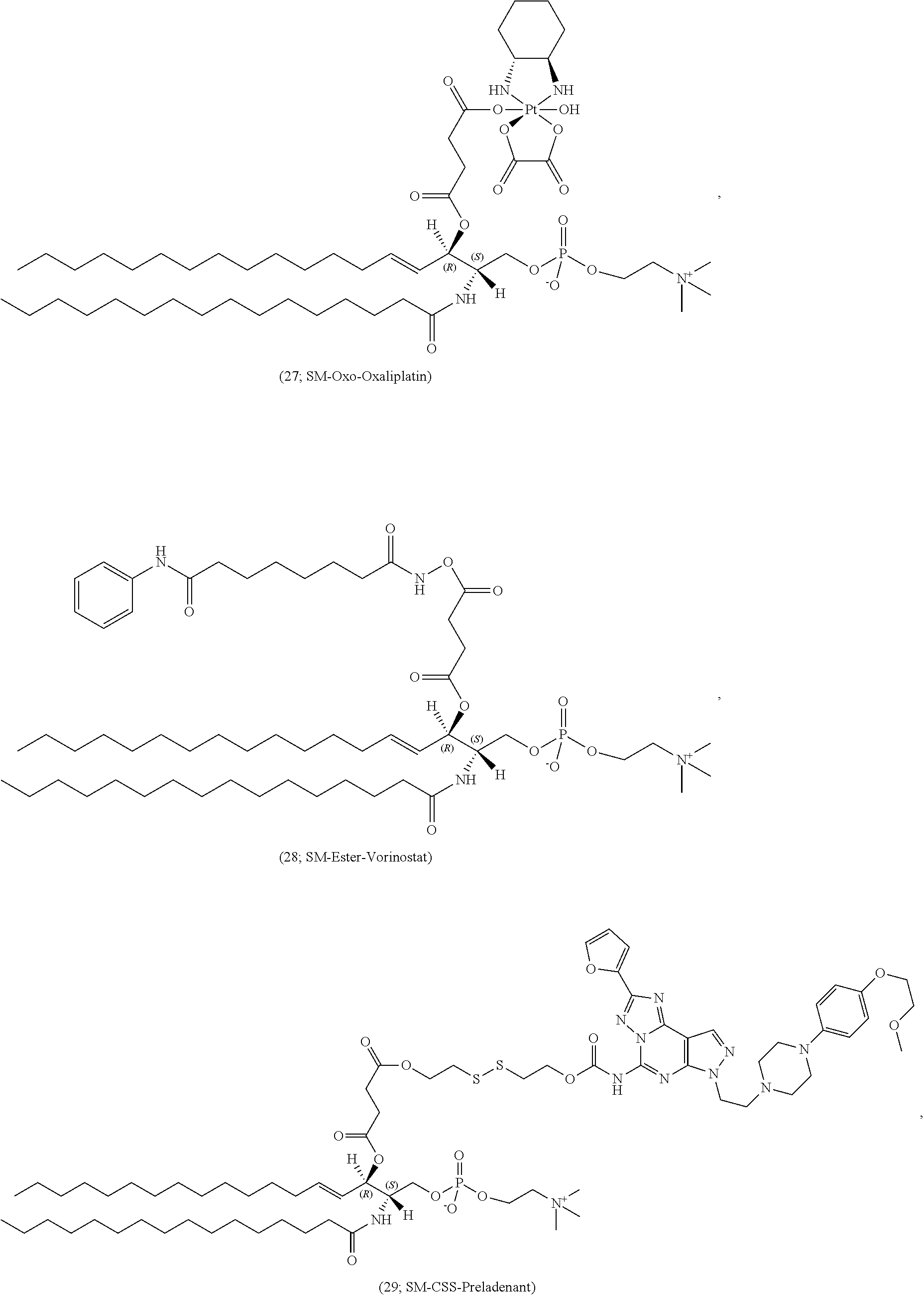

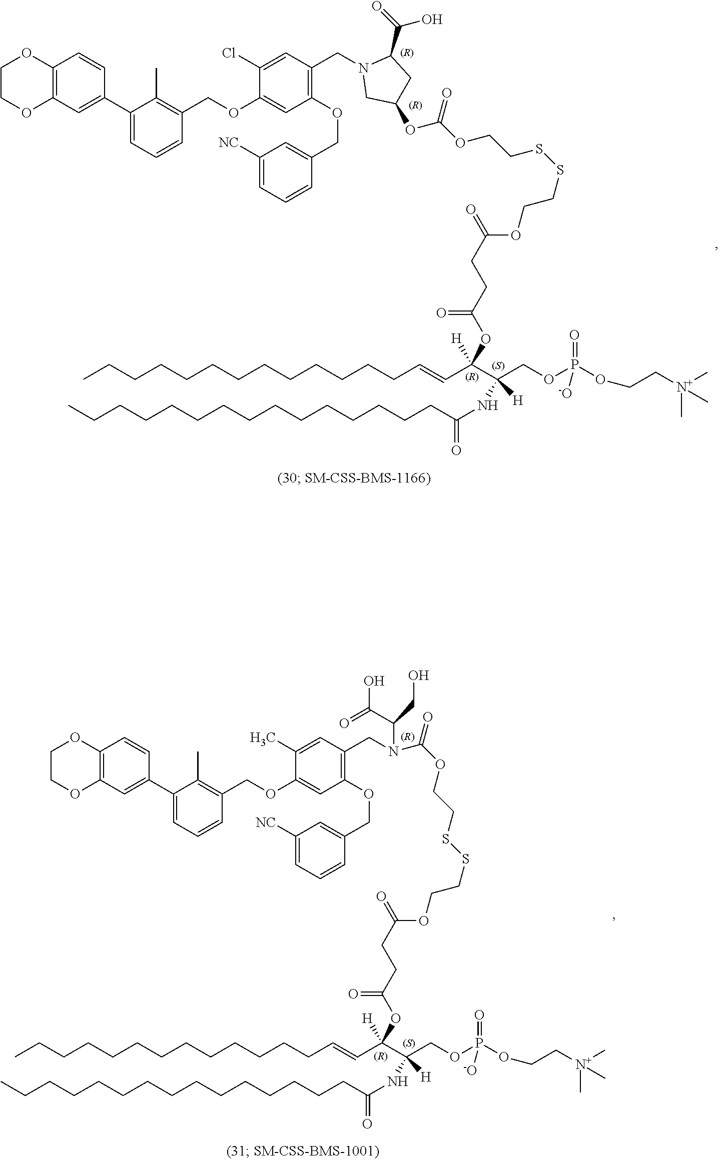

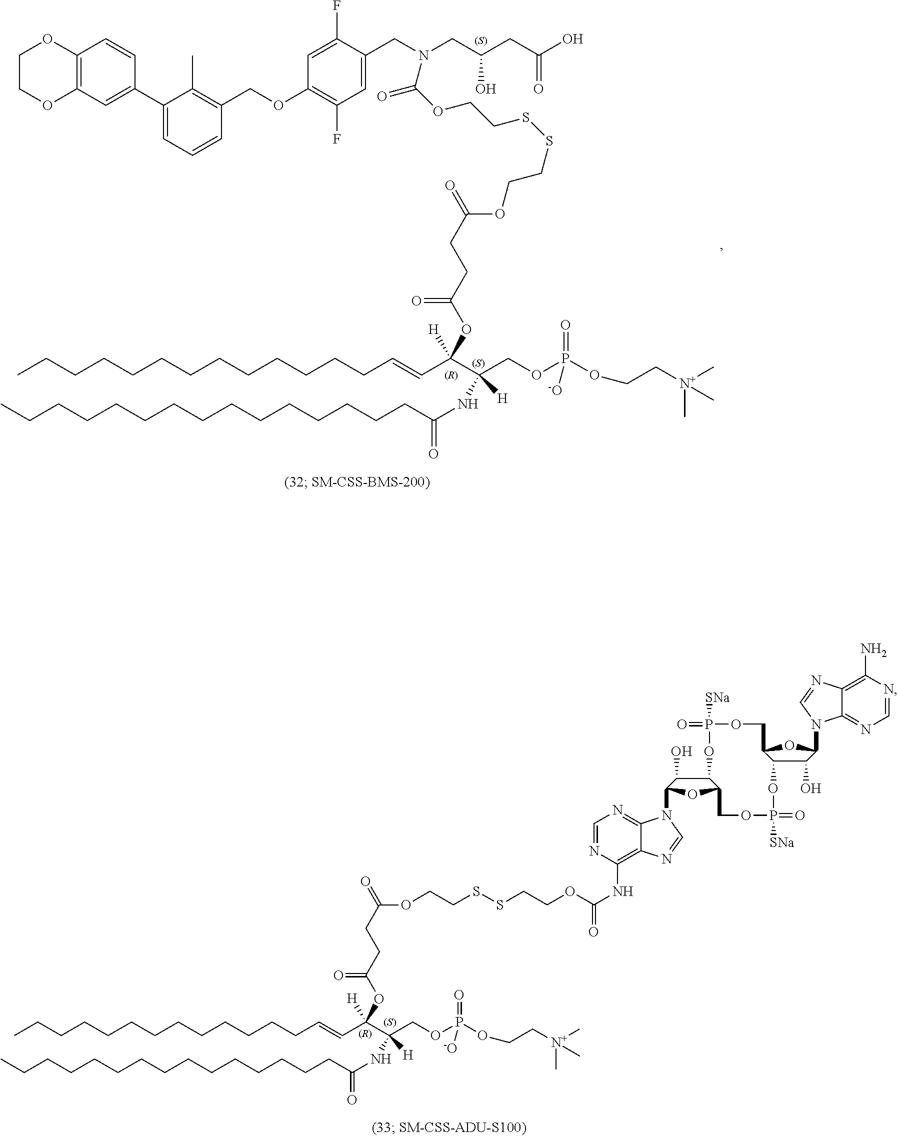

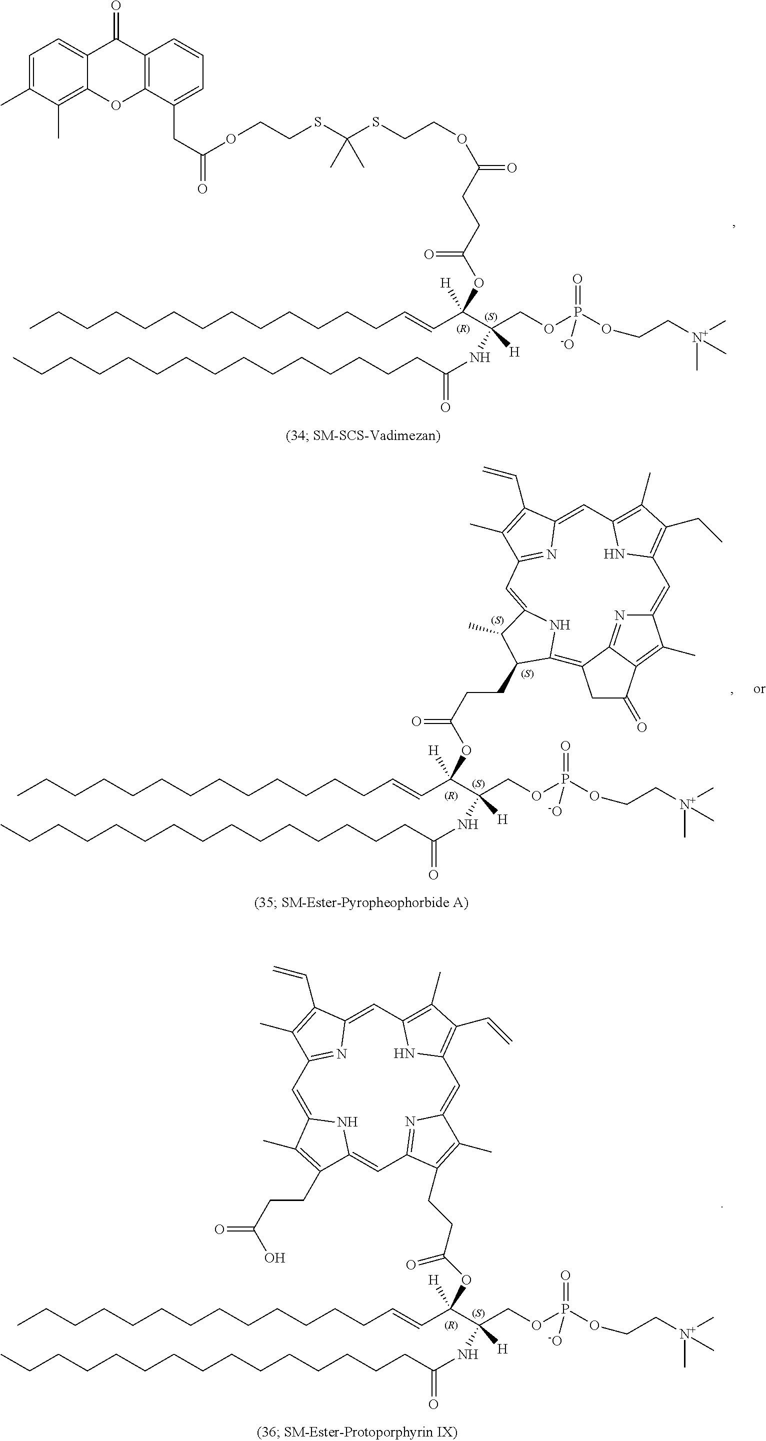

### Nanovesicles Comprising Sphingomyelin Drug Conjugates

Provided herein in certain embodiments, are nanovesicles formulated from one or more sphingomyelin (SM) drug conjugates set forth above. The sphingomyelin drug conjugates of the present disclosure can spontaneously self-assemble into nanovesicles, e.g., liposomal nanovesicles, in which the resulting nanovesicle can have a higher drug loading as compared to traditional liposomal therapeutic platforms.

The nanovesicles of the present disclosure can comprise liposomes having one or more membranes which comprise one or more sphingomyelin (SM) drug conjugates of the present disclosure. In some embodiments, the membrane can further comprise cholesterol.

In some embodiments, the drugs are either hydrophobic or hydrophilic and are incorporated within the lipid bilayer of the resulting nanovesicles.

In some embodiments, the nanovesicles comprise one or more sphingomyelin drug conjugates formulated as a nanovesicle. In certain of these embodiments, the one or more sphingomyelin drug conjugates is selected from the group consisting of: (1; SM-Ester-CPT), (2; SM-Glycine-CPT), (3; SM-SS-CPT), (4; SM-CSS-CPT), (5; SM-DMM-CPT), (6; SM-SCS-CPT), (7; SM-GFLG-CPT), (8; SM-NN-CPT), (9; SM-PLGLAG-CPT), (10; SM-AANK-CPT), (11; SM-B-SN-38), (12; SM-Ester-PTX), (13; SM-SS-Paclitaxel), (14; SM-CSS-Paclitaxel), (15; SM-Glycine-Paclitaxel), (16; SM-Ester-Docetaxel), (17; SM-SS-Docetaxel), (18; SM-CSS-Docetaxel), (19; SM-Glycine-Docetaxel), (20; SM-Ester-Epacadostat), (21; SM-B-(36; SM-Ester-Protoporphyrin IX). Bortezomib), (22; SM-CSS-Imatinib), (23; SM-CSS-Canertinib), (24; SM-CSS-Ceritinib), (25; SM-CSS-Dabrafenib), (26; SM-CSS-Vemurafenib), (27; SM-Oxo-Oxaliplatin), (28; SM-Ester-Vorinostat), (29; SM-CSS-Preladenant), (30; SM-CSS-BMS-1166), (31; SM-CSS-BMS-1001), (32; SM-CSS-BMS-200), (33; SM-CSS-ADU-S100), (34; SM-SCS-Vadimezan), (35; SM-Ester-Pyropheophorbide A), (36; SM-Ester-Protoporphyrin IX), and combinations thereof.

### Doxorubicin-Drug Conjugates

Provided herein in certain embodiments, are doxorubicin (DOX) drug conjugates comprising DOX, a linker moiety, and a drug. DOX, a chemotherapeutic drug, can serve as a membrane-crossing carrier that can convey the drug of the DOX-drug conjugate across the lipid bilayer of the one or more nanovesicles set forth above thereby, incorporating the drug into the one or more nanovesicles.

In some embodiments, the DOX drug conjugates of the present disclosure comprise a structure consistent with formulas (VII) and (VIII):

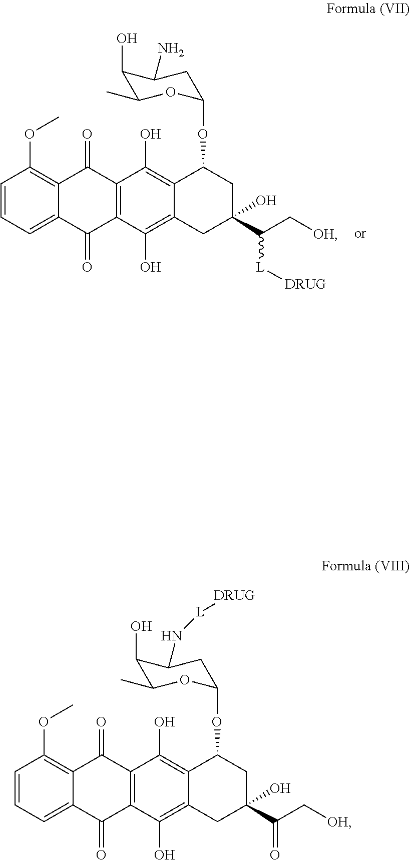

wherein:
L is a linker moiety; and
   Drug is an anti-cancer drug.

In some embodiments, the linker moiety (L) of formulas (VII)-(VIII) is selected from the group consisting of:

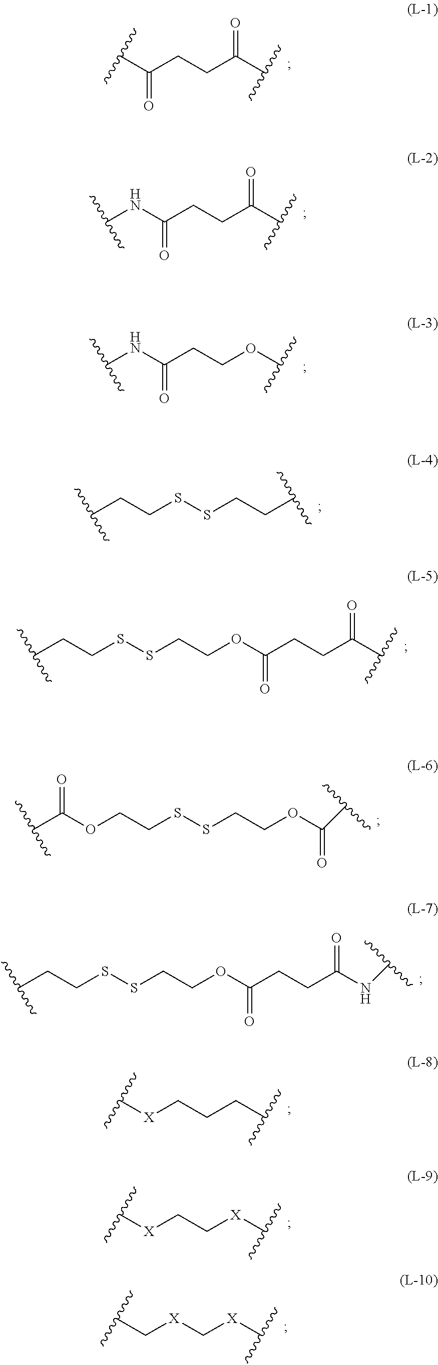

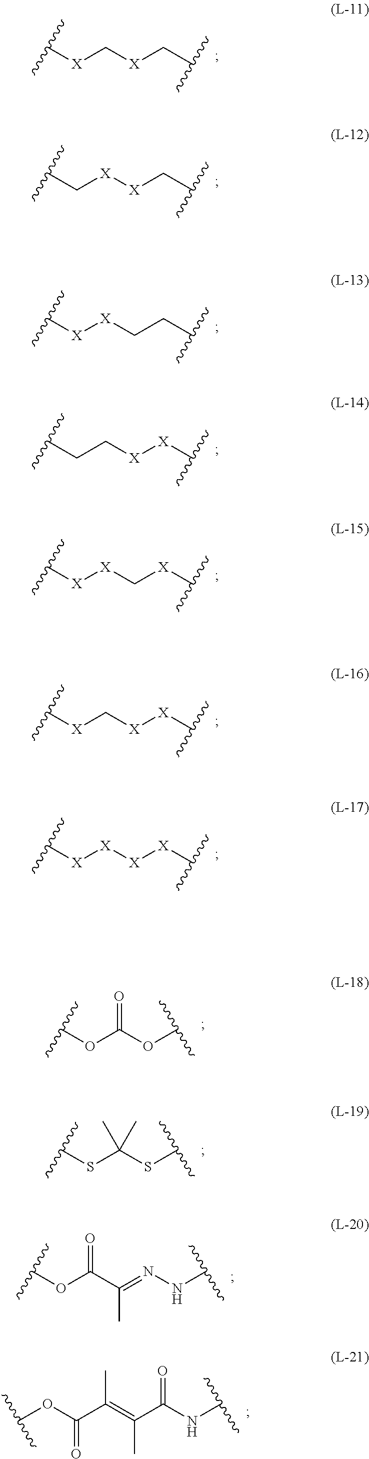

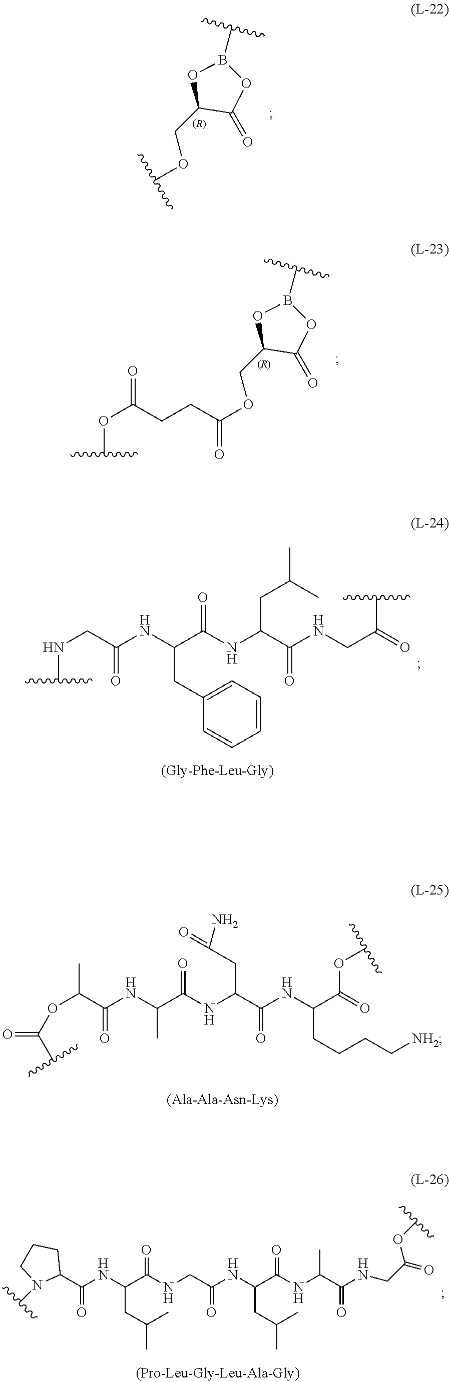

and combinations thereof,
wherein:
X is independently, O, S, —NH, or —CO.

In some embodiments, DOX-drug conjugates of the present disclosure comprise a chemical formula consistent with one or more of the following structures:

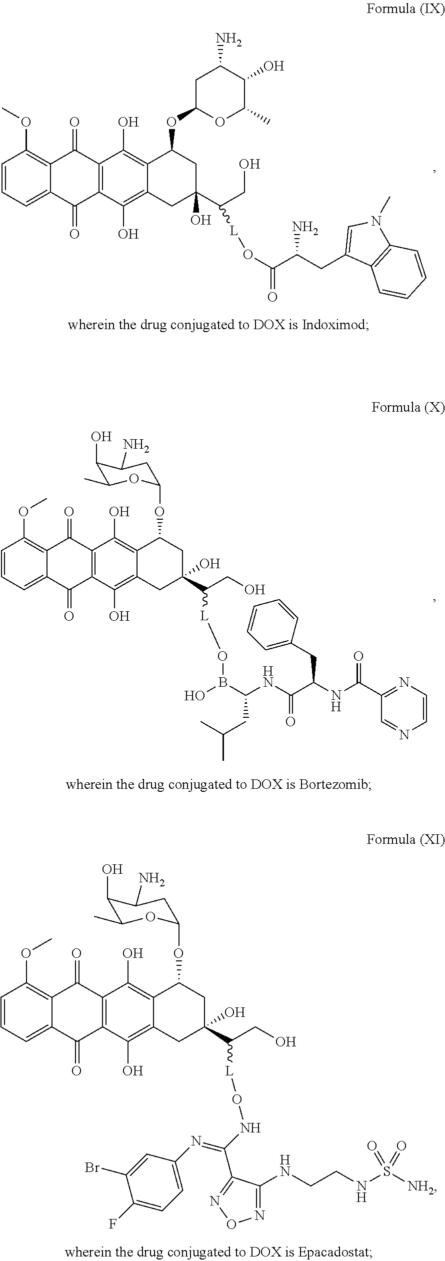

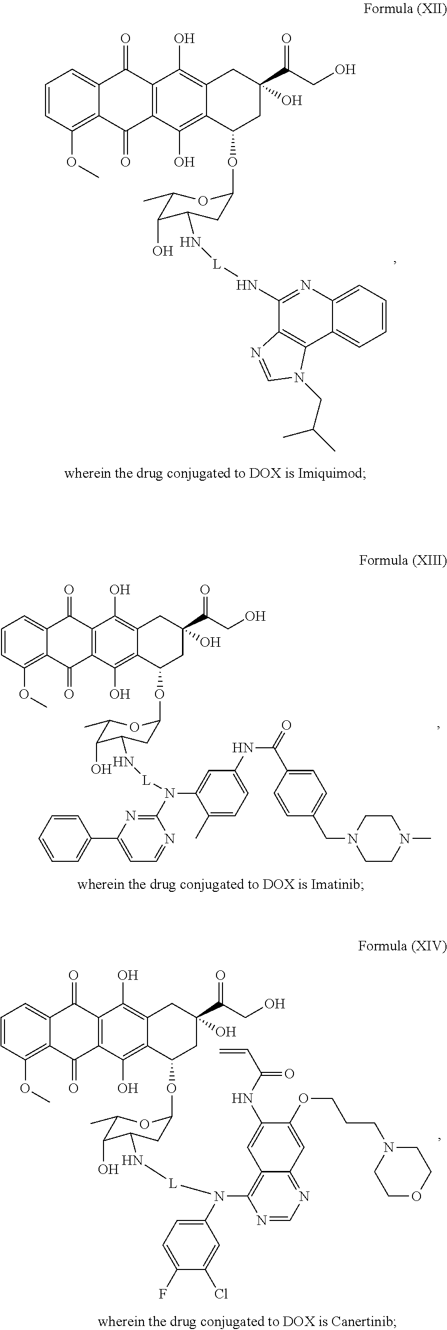

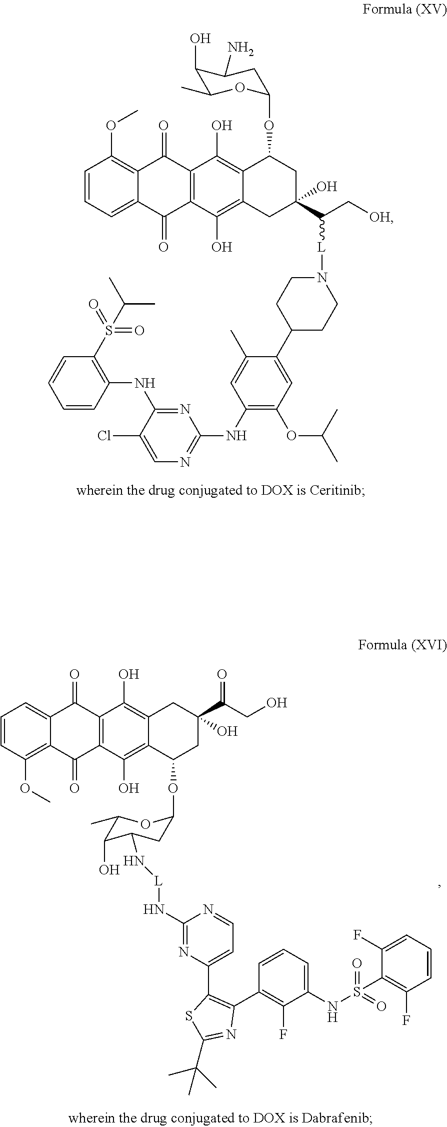

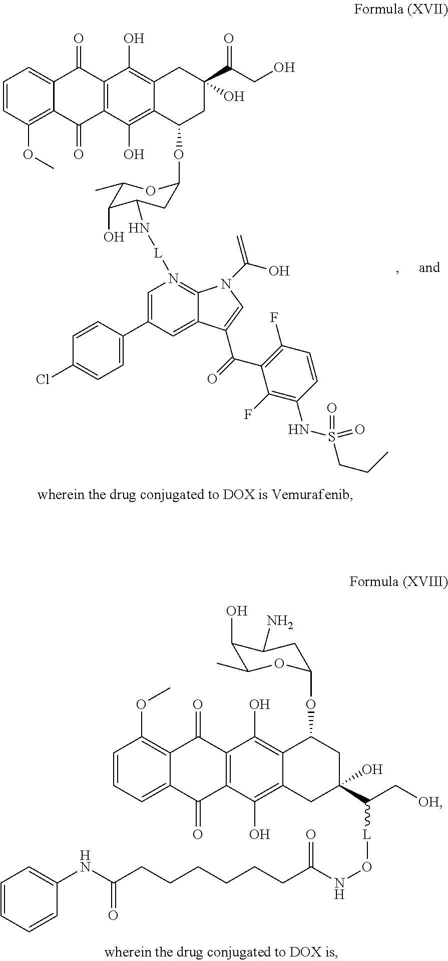

wherein L is a linker moiety.

In certain embodiments, the anti-cancer drug comprises a functional group moiety that permits conjugation to DOX via the linker moiety. In some embodiments, the functional group moiety is selected from —COOH, —OH, —NH₂and/or C═O. In some embodiments, the anti-cancer drug is a hydrophobic or hydrophilic drug. In certain of these embodiments, the anti-cancer drug is an IDO1 inhibitor, e.g., indoximod (IND).

Non-limiting examples of suitable anti-cancer drugs include IND, bortezomib, epacadostat, imiquimod, imatinib, canertinib, ceritinib, dabrafenib, vemurafenib, vorinostat, ADU-S100, amrubicin, AZD4635, BMS-1001, BMS-1166, BMS-200, BMS202, BMS-242, CA170, cabazitaxel, cabozantinib, cam ptothecin, capecitabine, carboplatin, cisplatin, dacarbazine, darolutamide, degarelix, digitoxin, digoxin, docetaxel, eribulin, etoposide, 5-fluorouracil, gemcitabine, irinotecan, ixabepilone, lenvatinib, methotrexate, mitoxantrone, NIR178, NLG919, oxaliplatin, paclitaxel, pazopanib, pemetrexed, preladenant, septacidin, SN-38, sorafenib, streptozocin, sunitinib, temozolomide, tipiracil, trifluridine, vadimezan, vinblastine, vincristine, vinorelbine, vipadenant, or combinations thereof.

In yet another embodiment, DOX-drug conjugates of the present disclosure comprise a chemical formula consistent with one or more of the following structures:

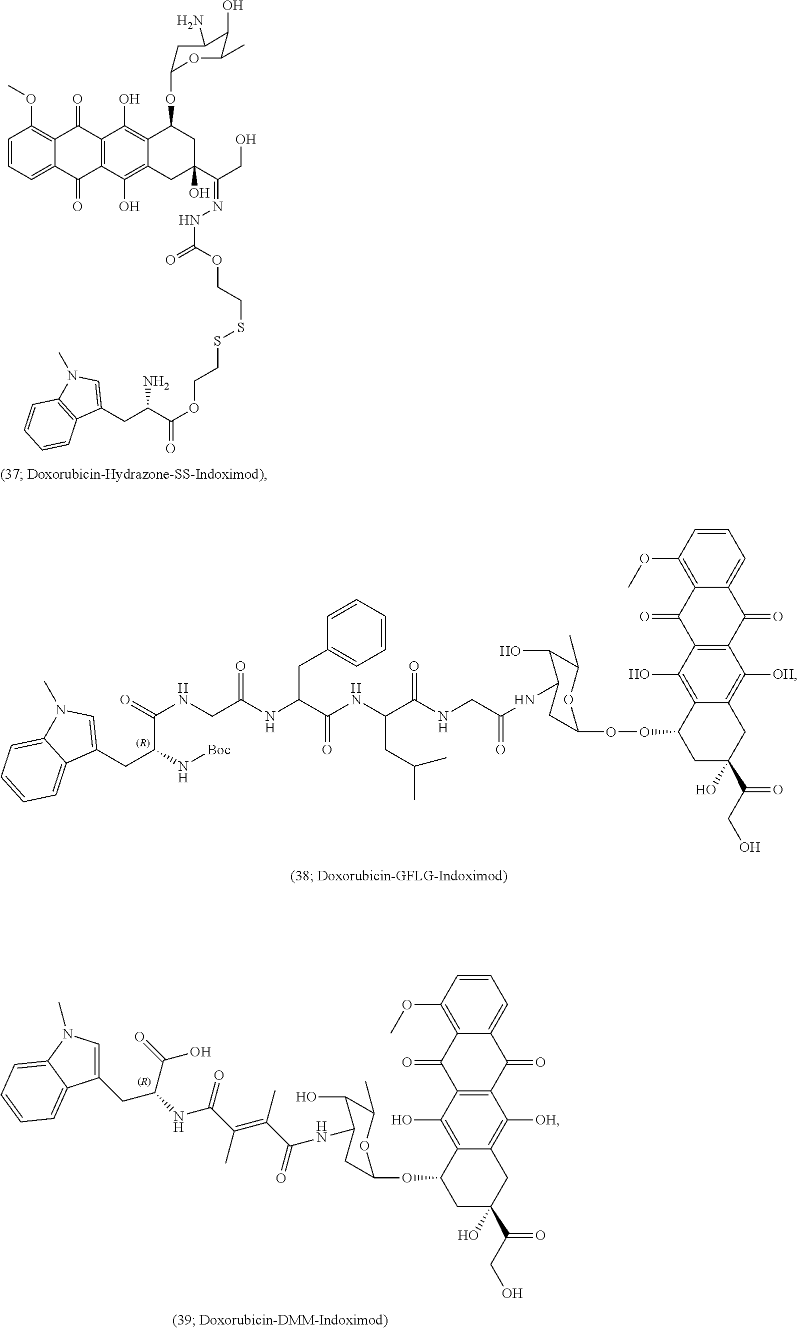

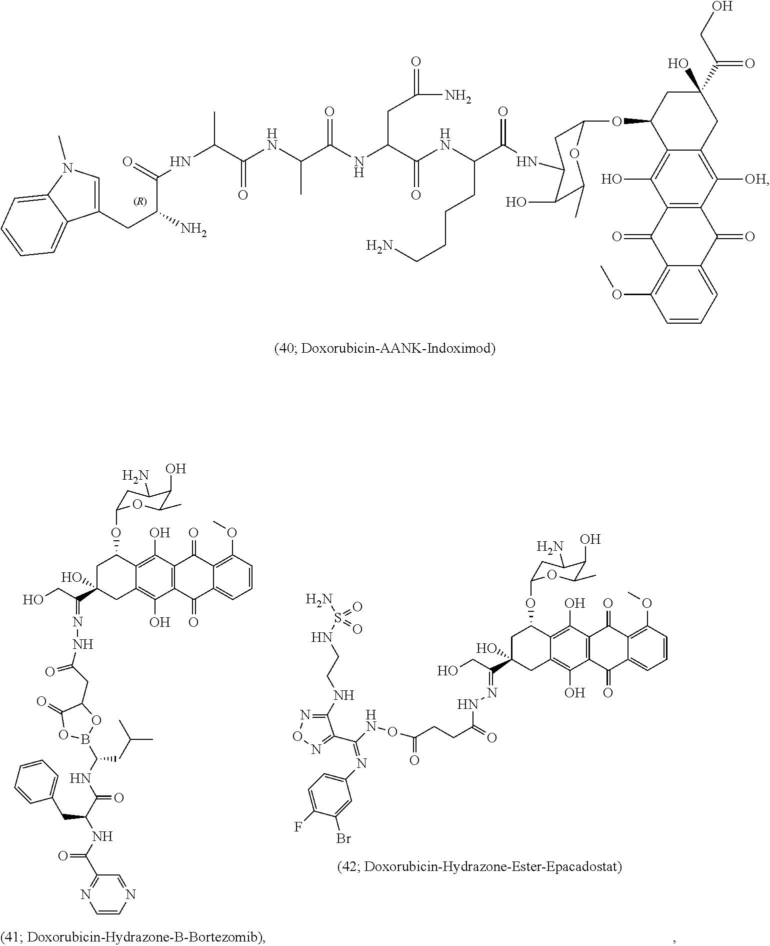

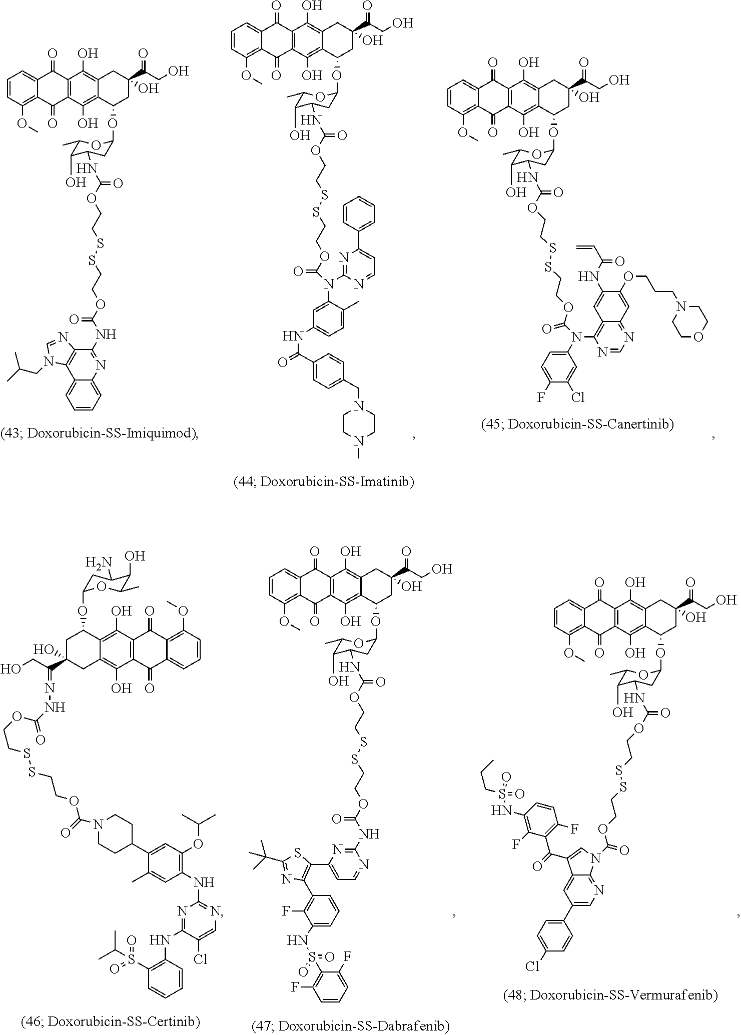

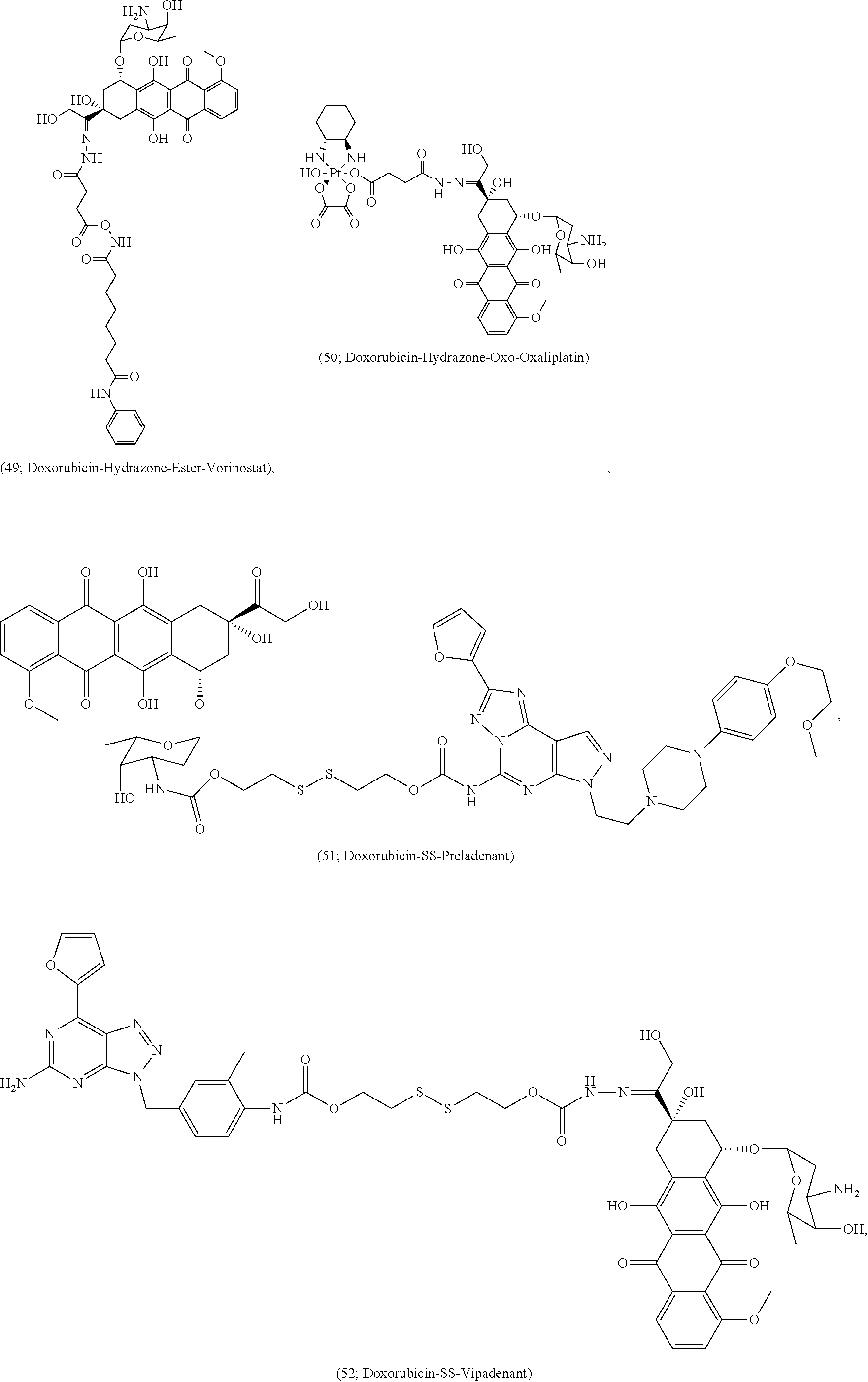

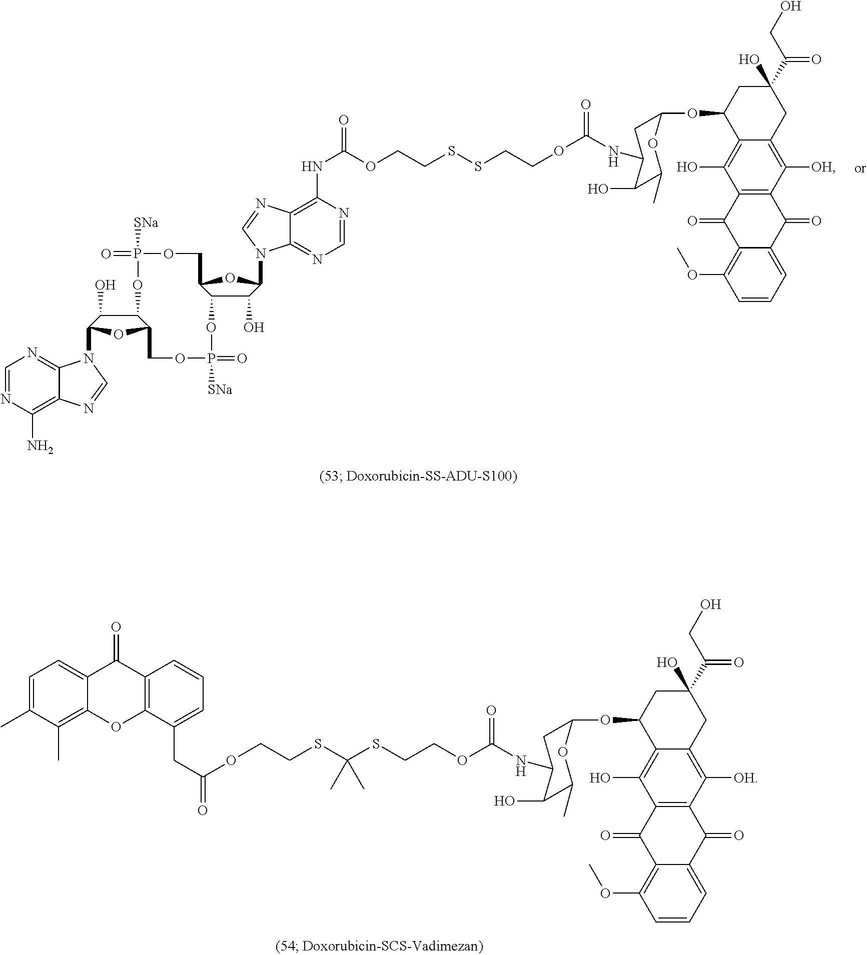

### Nanovesicles Comprising Sphingomyelin Drug Conjugates and Doxorubicin-Drug Conjugates

Provided herein in certain embodiments, are nanovesicles formulated from one or more sphingomyelin (SM) drug conjugates set forth above and comprising one or more DOX-drug conjugates set forth above. The DOX of the DOX-drug conjugate can serve as a membrane-crossing carrier to bring a drug of the DOX-drug conjugate into the interior of the nanovesicle, resulting in a nanovesicle comprising both hydrophobic and hydrophilic drugs. The resulting nanovesicle can then synergistically co-deliver both the hydrophobic and hydrophilic drugs, where this co-encapsulation has not been achieved in existing liposomal platforms.

The nanovesicles of the present disclosure can comprise liposomes having one or more membranes which comprise one or more sphingomyelin (SM) drug conjugates of the present disclosure. In some embodiments, the membrane can further comprise cholesterol. The nanovesicles further comprise an interior core surrounded by the one or more membranes.

In some embodiments, the nanovesicle comprises a sphingomyelin drug conjugate and a DOX-drug conjugate, wherein the sphingomyelin drug conjugate is incorporated into a bilayer of the nanovesicle and the DOX-drug conjugate is incorporated into an interior core of the nanovesicle.

In some embodiments, the drug associated with the sphingomyelin (SM) drug conjugate is hydrophilic and the drug associated with the DOX-drug conjugate is hydrophobic. In another embodiment, the drug associated with the sphingomyelin (SM) drug conjugate is hydrophobic and the drug associated with the DOX-drug conjugate is hydrophobic. In another embodiment, the drug associated with the sphingomyelin (SM) drug conjugate is hydrophilic and the drug associated with the DOX-drug conjugate is hydrophilic. In certain embodiments, the drug associated with the sphingomyelin (SM) drug conjugate is hydrophobic and the drug associated with the DOX-drug conjugate is hydrophobic.

In some embodiments, after crossing the lipid bilayer, the drug of the DOX-drug conjugate precipitates from the DOX-drug conjugate, releasing both DOX and the drug into the interior of the nanovesicle. Precipitation of the drug can prevent drug leakage from the interior of the nanovesicle.

In some embodiments, the nanovesicle is further conjugated to one or more tumor targeting ligands that can further improve the intratumoral uptake and antitumor efficacy. Tumor targeting ligand can comprise one or more of small molecules to include: folate or folic acid, anisamide, phenylboronic acid, glycyrrhizic acid, pamidronic acid, triphenylphosphine, flavin mononucleotide; Polysaccharides: hyaluronic acid, galactose, chitosan, mannose, heparin, dextran, N-acetyl-β-D-galactosamine, sialic acid, lactobionic acid; Proteins: transferrin, EGFP-EGF1, AopB, ApoE, lactoferrin, tumor necrosis factor (TNF)-related apoptosis-inducing ligand (TRAIL); Antibodies: intercellular adhesion molecule 1 antibody (ICAM-1), CD44 antibody, EGFR antibody (cetuximab, panitumumab), PD-L1 antibody, EpCAM antibody, EphA10 antibody, AFP antibody, AMG655 antibody; Peptides: arginine-glycine-aspartate (RGD), asparagine-glycine-arginine (NGR), melittin (Mel), MT peptide, T7 peptide, Cell-penetrating peptides (CPP), Gly-Sar, mitochondria) targeting peptide (pALDH Leader), K237 peptide, YIGSR peptide, poly(histidine-arginine)6 (H6R6), angiopep-2, octreotide, pardaxin, Fragment C of tetanus toxin (TTC); Aptamers: aptamer S6, aptamer GBI-10, aptamer AS1411, aptamer RP, aptamer R8, aptamer AraHH036, aptamer MUC1, aptamer PSMA, aptamer EpCAM, or combinations thereof.

In some embodiments, the nanovesicles comprise one or more sphingomyelin drug conjugates, wherein the drug of the sphingomyelin drug conjugates is a hydrophobic or hydrophilic drug and is incorporated within a lipid biolayer of the nanovesicles. In certain of these embodiments, the nanovesicles further comprise one or more DOX-drug conjugates, wherein the drug of the DOX-drug conjugate is hydrophilic or hydrophobic and is incorporated into an interior of the nanovesicles.

In some embodiments, the nanovesicles comprise one or more sphingomyelin drug conjugates formulated as a nanovesicle. In certain of these embodiments, the one or more sphingomyelin drug conjugates is selected from the group consisting of: (1; SM-Ester-CPT), (2; SM-Glycine-CPT), (3; SM-SS-CPT), (4; SM-CSS-CPT), (5; SM-DMM-CPT), (6; SM-SCS-CPT), (7; SM-GFLG-CPT), (8; SM-NN-CPT), (9; SM-PLGLAG-CPT), (10; SM-AANK-CPT), (11; SM-B-SN-38), (12; SM-Ester-PTX), (13; SM-SS-Paclitaxel), (14; SM-CSS-Paclitaxel), (15; SM-Glycine-Paclitaxel), (16; SM-Ester-Docetaxel), (17; SM-SS-Docetaxel), (18; SM-CSS-Docetaxel), (19; SM-Glycine-Docetaxel), (20; SM-Ester-Epacadostat), (21; SM-B-Bortezomib), (22; SM-CSS-Imatinib), (23; SM-CSS-Canertinib), (24; SM-CSS-Ceritinib), (25; SM-CSS-Dabrafenib), (26; SM-CSS-Vemurafenib), (27; SM-Oxo-Oxaliplatin), (28; SM-Ester-Vorinostat), (29; SM-CSS-Preladenant), (30; SM-CSS-BMS-1166), (31; SM-CSS-BMS-1001), (32; SM-CSS-BMS-200), (33; SM-CSS-ADU-S100), (34; SM-SCS-Vadimezan), (35; SM-Ester-Pyropheophorbide A), (36; SM-Ester-Protoporphyrin IX), and combinations thereof.

In some embodiments, the nanovesicles comprise one or more one or more DOX-drug conjugates, wherein the one or more DOX-drug conjugates is incorporated into a lumen of the nanovesicles. In certain of these embodiments, the one or more DOX-drug conjugates is selected from the group consisting of: (37; Doxorubicin-Hydrazone-SS-Indoximod), (38; Doxorubicin-GFLG-Indoximod), (39; Doxorubicin-DMM-Indoximod), (40; Doxorubicin-AANK-Indoximod), (41; Doxorubicin-Hydrazone-B-Bortezomib), (42; Doxorubicin-Epacadostat), (43; Doxorubicin-SS-Imiquimod), (44; Doxorubicin-SS-Imatinib), (45; Doxorubicin-SS-Canertinib), (46; Doxorubicin-SS-Ceritinib), (47; Doxorubicin-SS-Dabrafenib), (48; Doxorubicin-SS-Vemurafenib), (49; Doxorubicin-Hydrazone-Ester-Vorinostat), (50; Doxorubicin-Oxo-Oxaliplatin), (51; Doxorubicin-SS-Preladenant), (52; Doxorubicin-SS-Vipadenant), (53; Doxorubicin-SS-ADU-S100), or (54; Doxorubicin-SCS-Vadimezan). In another aspect, the therapeutic nanoparticle system further comprises (37; Doxorubicin-Hydrazone-SS-Indoximod).

In certain embodiments, the nanovesicle comprises one or more one or more sphingomyelin drug conjugates formulated as nanovesicle selected from the group consisting of: (1; SM-Ester-CPT), (2; SM-Glycine-CPT), (3; SM-SS-CPT), (4; SM-CSS-CPT), (5; SM-DMM-CPT), (6; SM-SCS-CPT), (7; SM-GFLG-CPT), (8; SM-NN-CPT), (9; SM-PLGLAG-CPT), (10; SM-AANK-CPT), (11; SM-B-SN-38), (12; SM-Ester-PTX), (13; SM-SS-Paclitaxel), (14; SM-CSS-Paclitaxel), (15; SM-Glycine-Paclitaxel), (16; SM-Ester-Docetaxel), (17; SM-SS-Docetaxel), (18; SM-CSS-Docetaxel), (19; SM-Glycine-Docetaxel), (20; SM-Ester-Epacadostat), (21; SM-B-Bortezomib), (22; SM-CSS-Imatinib), (23; SM-CSS-Canertinib), (24; SM-CSS-Ceritinib), (25; SM-CSS-Dabrafenib), (26; SM-CSS-Vemurafenib), (27; SM-Oxo-Oxaliplatin), (28; SM-Ester-Vorinostat), (29; SM-CSS-Preladenant), (30; SM-CSS-BMS-1166), (31; SM-CSS-BMS-1001), (32; SM-CSS-BMS-200), (33; SM-CSS-ADU-S100), (34; SM-SCS-Vadimezan), (35; SM-Ester-Pyropheophorbide A), and (36; SM-Ester-Protoporphyrin IX). In certain of these embodiments, the nanovesicle further comprises comprising one or more DOX-drug conjugates incorporated into an interior of the nanovesicles selected from the group consisting of: (37; Doxorubicin-Hydrazone-SS-Indoximod), (38; Doxorubicin-GFLG-Indoximod), (39; Doxorubicin-DMM-Indoximod), (40; Doxorubicin-AANK-Indoximod), (41; Doxorubicin-Hydrazone-B-Bortezomib), (42; Doxorubicin-Epacadostat), (43; Doxorubicin-SS-Imiquimod), (44; Doxorubicin-SS-Imatinib), (45; Doxorubicin-SS-Canertinib), (46; Doxorubicin-SS-Ceritinib), (47; Doxorubicin-SS-Dabrafenib), (48; Doxorubicin-SS-Vemurafenib), (49; Doxorubicin-Hydrazone-Ester-Vorinostat), (50; Doxorubicin-Oxo-Oxaliplatin), (51; Doxorubicin-SS-Preladenant), (52; Doxorubicin-SS-Vipadenant), (53; Doxorubicin-SS-ADU-S100), or (54; Doxorubicin-SCS-Vadimezan). In one aspect, the combination comprises compound (4 SM-CSS-CPT)and compound (37; Doxorubicin-Hydrazone-SS-Indoximod).

Table 1 provides structures of the drugs of the sphingomyelin drug conjugates and/or DOX-drug conjugates provided herein.

TABLE 1
Drugs and associated structures of the sphingomyelin drug conjugates and/or DOX-drug conjugates provided herein.

| Drug Name | Structure |
|---|---|
| Camptothecin | 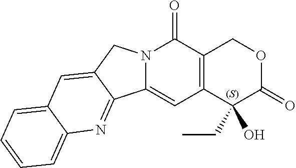 |
| Paclitaxel | 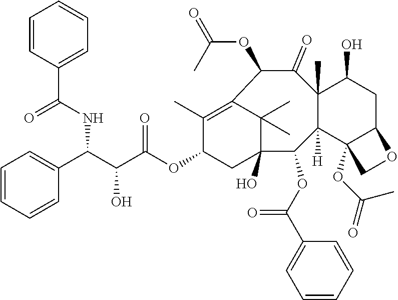 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O] |
| Docetaxel | 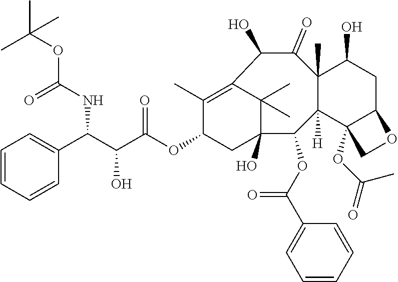 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C] |
| ADU-S100 | 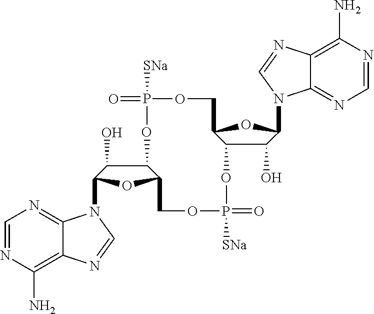 |
| Amrubicin | 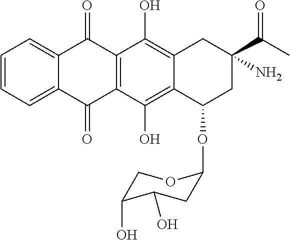 [SMILES: CC(=O)[C@]1(N)Cc2c(O)c3c(c(O)c2[C@@H](OC2CC(O)C(O)CO2)C1)C(=O)c1ccccc1C3=O] |
| 5-aminolevulinic acid | 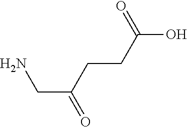 |
| AZD4635 | 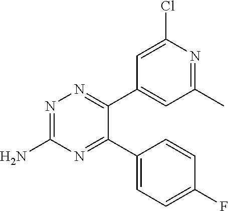 [SMILES: Cc1cc(-c2nnc(N)nc2-c2ccc(F)cc2)cc(Cl)n1] |
| BMS-1001 | 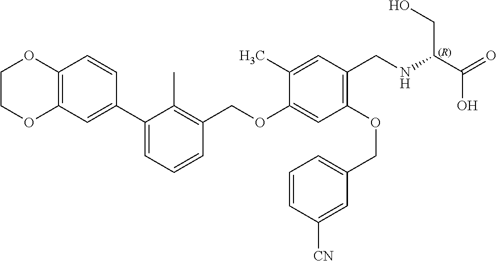 |
| BMS-1166 | 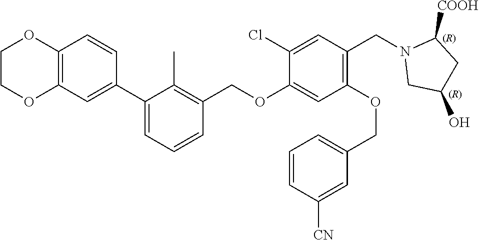 |
| BMS-200 | 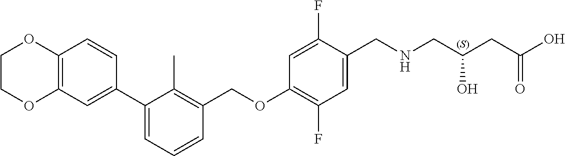 |
| BMS-202 | 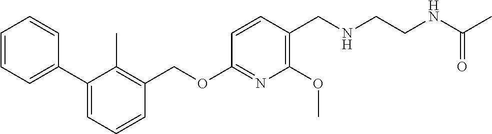 [SMILES: COc1nc(OCc2cccc(-c3ccccc3)c2C)ccc1CNCCNC(C)=O] |
| BMS-242 | 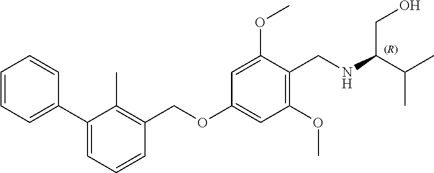 |
| Bortezomib | 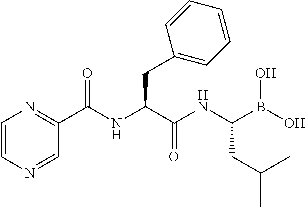 [SMILES: CC(C)C[C@H](NC(=O)[C@H](Cc1ccccc1)NC(=O)c1cnccn1)B(O)O] |
| CA170 | 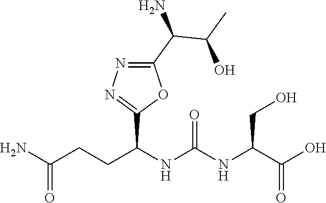 [SMILES: C[C@@H](O)[C@H](N)c1nnc([C@H](CCC(N)=O)NC(=O)N[C@@H](CO)C(=O)O)o1] |
| Cabazitaxel | 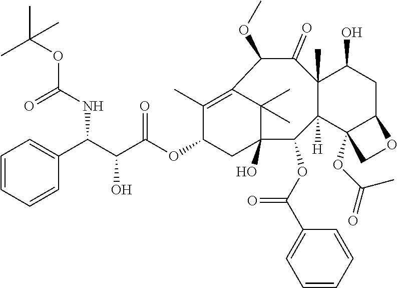 [SMILES: CO[C@H]1C(=O)[C@@]2(C)[C@H]([C@H](OC(=O)c3ccccc3)[C@]3(O)C[C@H](OC(=O)[C@H](O)[C@@H](NC(=O)OC(C)(C)C)c4ccccc4)C(C)=C1C3(C)C)[C@]1(OC(C)=O)CO[C@@H]1C[C@@H]2O] |
| Cabozantinib | 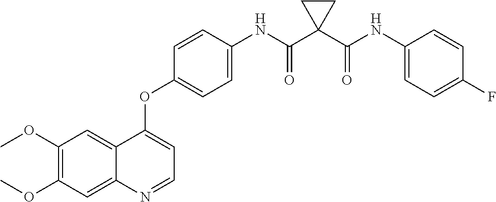 [SMILES: COc1cc2nccc(Oc3ccc(NC(=O)C4(C(=O)Nc5ccc(F)cc5)CC4)cc3)c2cc1OC] |
| Canertinib | 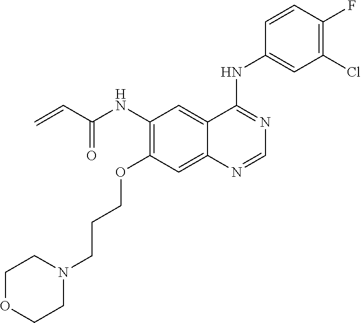 [SMILES: C=CC(=O)Nc1cc2c(Nc3ccc(F)c(Cl)c3)ncnc2cc1OCCCN1CCOCC1] |
| Capecitabine | 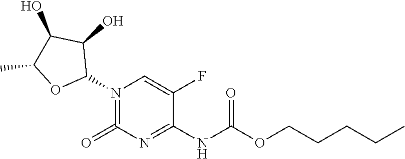 |
| Carboplatin | 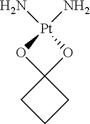 |
| Ceritinib | 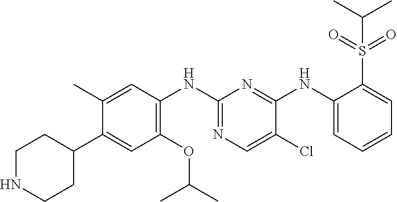 [SMILES: Cc1cc(Nc2ncc(Cl)c(Nc3ccccc3S(=O)(=O)C(C)C)n2)c(OC(C)C)cc1C1CCNCC1] |
| Chlorine6 | 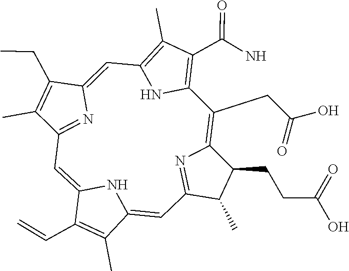 [SMILES: C=Cc1c(C)c2cc3nc(c(CC(=O)O)c4[nH]c(cc5nc(cc1[nH]2)C(C)=C5CC)c(C)c4C(N)=O)[C@@H](CCC(=O)O)[C@@H]3C] |
| Cisplatin | 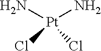 |
| Dabrafenib | 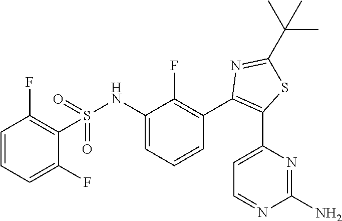 [SMILES: CC(C)(C)c1nc(-c2cccc(NS(=O)(=O)c3c(F)cccc3F)c2F)c(-c2ccnc(N)n2)s1] |
| Dacarbazine | 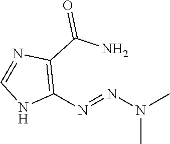 |
| Darolutamide | 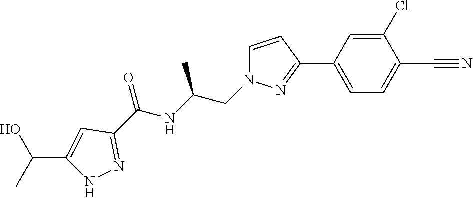 [SMILES: CC(O)c1cc(C(=O)N[C@@H](C)Cn2ccc(-c3ccc(C#N)c(Cl)c3)n2)n[nH]1] |
| Daunorubicin | 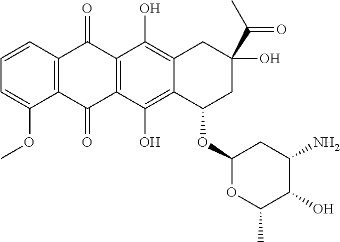 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1] |
| Degarelix | 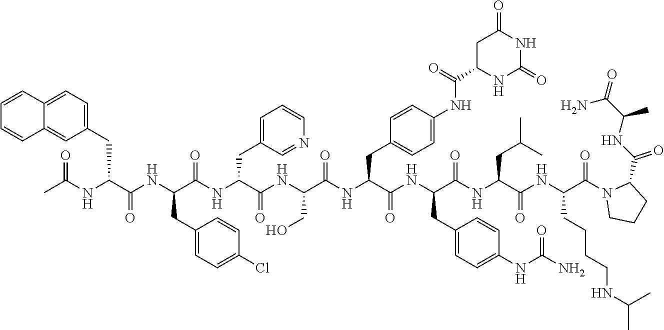 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O] |
| Digoxin | 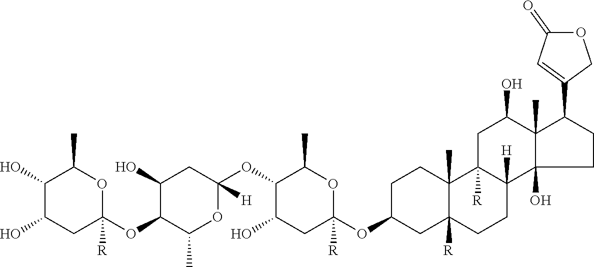 |
| Doxorubicin | 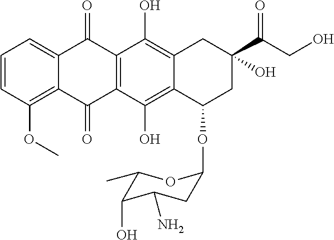 [SMILES: COc1cccc2c1C(=O)c1c(O)c3c(c(O)c1C2=O)C[C@@](O)(C(=O)CO)C[C@@H]3OC1CC(N)C(O)C(C)O1] |
| Epacadostat | 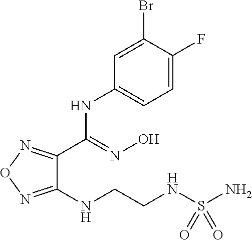 [SMILES: NS(=O)(=O)NCCNc1nonc1/C(=N/O)Nc1ccc(F)c(Br)c1] |
| Epirubicin | 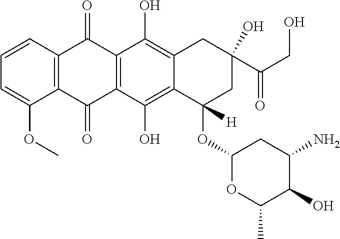 [SMILES: COc1cccc2c1C(=O)c1c(O)c3c(c(O)c1C2=O)C[C@@](O)(C(=O)CO)C[C@@H]3O[C@@H]1C[C@H](N)[C@@H](O)[C@H](C)O1] |
| Eribulin | 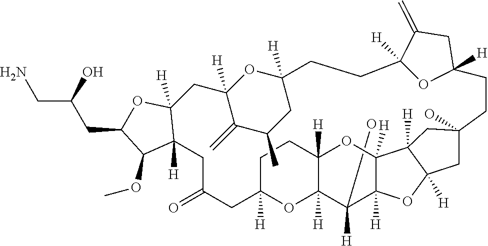 [SMILES: C=C1C[C@@H]2CC[C@]3(O)C[C@@H]4[C@@H]5O[C@H]6CC[C@H](CC(=O)C[C@@H]7[C@@H](OC)[C@@H](C[C@H](O)CN)O[C@H]7C[C@H]7O[C@@H](CC[C@@H]1O2)C[C@@H](C)C7=C)O[C@@H]6[C@H](O)[C@@H]5O[C@@H]4C3] |
| Esorubicin | 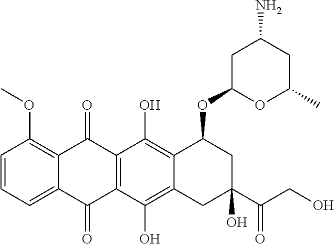 [SMILES: COc1cccc2c1C(=O)c1c(O)c3c(c(O)c1C2=O)C[C@@](O)(C(=O)CO)C[C@@H]3O[C@H]1C[C@H](N)C[C@H](C)O1] |
| Etoposide | 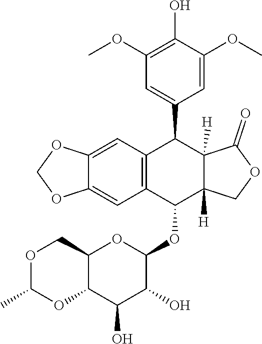 |
| Fingolimod | 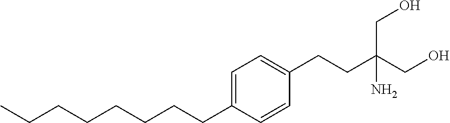 [SMILES: CCCCCCCCc1ccc(CCC(N)(CO)CO)cc1] |
| 5-fluorouracil | 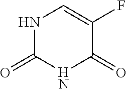 |
| Galanthamine | 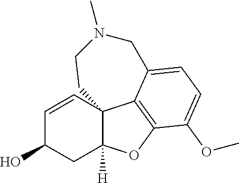 [SMILES: COc1ccc2c3c1O[C@H]1C[C@@H](O)C=C[C@@]31CCN(C)C2] |
| Gemcitabine | 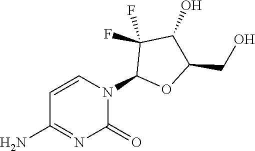 |
| Idarubicin | 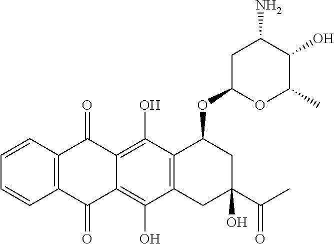 [SMILES: CC(=O)[C@]1(O)Cc2c(O)c3c(c(O)c2[C@@H](O[C@H]2C[C@H](N)[C@H](O)[C@H](C)O2)C1)C(=O)c1ccccc1C3=O] |
| Imatinib | 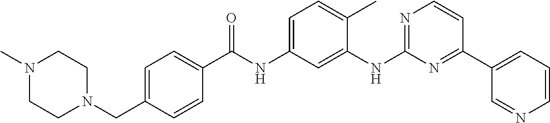 [SMILES: Cc1ccc(NC(=O)c2ccc(CN3CCN(C)CC3)cc2)cc1Nc1nccc(-c2cccnc2)n1] |
| Imiquimod | 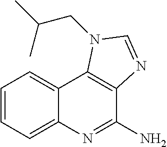 [SMILES: CC(C)Cn1cnc2c(N)nc3ccccc3c21] |
| Indoximod | 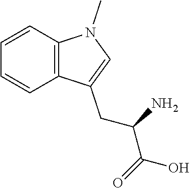 [SMILES: Cn1cc(C[C@@H](N)C(=O)O)c2ccccc21] |
| Irinotecan | 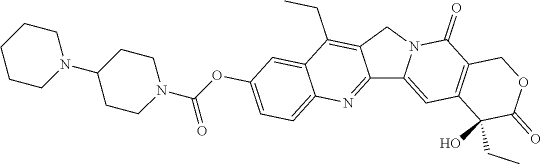 [SMILES: CCc1c2c(nc3ccc(OC(=O)N4CCC(N5CCCCC5)CC4)cc13)-c1cc3c(c(=O)n1C2)COC(=O)[C@]3(O)CC] |
| Ixabepilone | 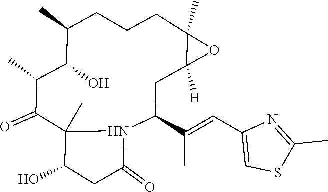 [SMILES: C/C(=C\c1csc(C)n1)[C@@H]1C[C@@H]2O[C@]2(C)CCC[C@H](C)[C@H](O)[C@@H](C)C(=O)C2(C)[C@@H](O)CC(=O)N12] |
| Lenvatinib | 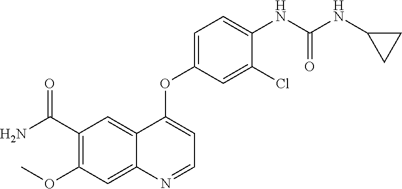 [SMILES: COc1cc2nccc(Oc3ccc(NC(=O)NC4CC4)c(Cl)c3)c2cc1C(N)=O] |
| Memantine | 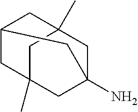 |
| Methotrexate | 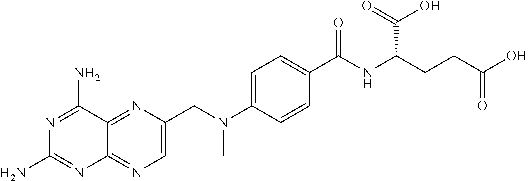 [SMILES: CN(Cc1cnc2nc(N)nc(N)c2n1)c1ccc(C(=O)N[C@@H](CCC(=O)O)C(=O)O)cc1] |
| Mitoxantrone | 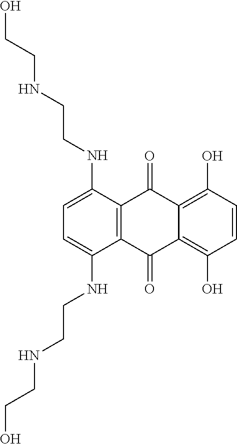 [SMILES: O=C1c2c(O)ccc(O)c2C(=O)c2c(NCCNCCO)ccc(NCCNCCO)c21] |
| NIR178 | 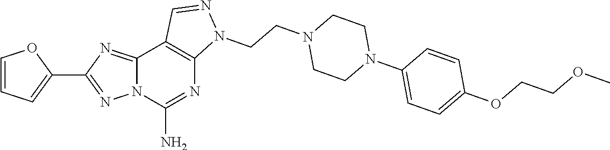 [SMILES: COCCOc1ccc(N2CCN(CCn3ncc4c3nc(N)n3nc(-c5ccco5)nc43)CC2)cc1] |
| NLG919 | 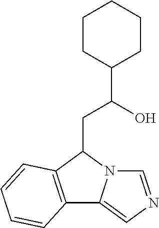 [SMILES: OC(CC1c2ccccc2-c2cncn21)C1CCCCC1] |
| Oxaliplatin | 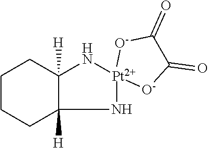 |
| Pazopanib | 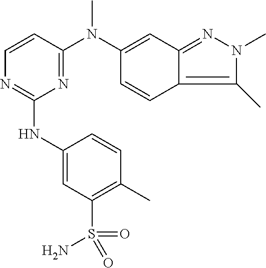 [SMILES: Cc1ccc(Nc2nccc(N(C)c3ccc4c(C)n(C)nc4c3)n2)cc1S(N)(=O)=O] |
| Pemetrexed | 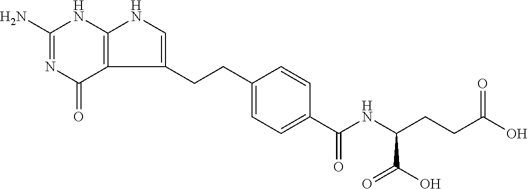 [SMILES: Nc1nc(=O)c2c(CCc3ccc(C(=O)N[C@@H](CCC(=O)O)C(=O)O)cc3)c[nH]c2[nH]1] |
| Preladenant | 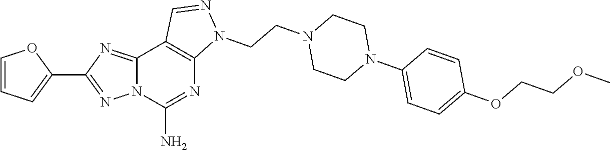 [SMILES: COCCOc1ccc(N2CCN(CCn3ncc4c3nc(N)n3nc(-c5ccco5)nc43)CC2)cc1] |
| Protoporphyrin IX | 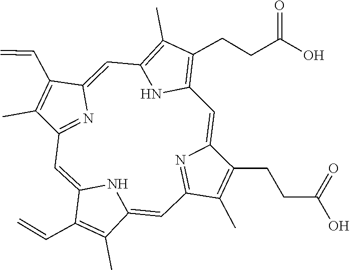 [SMILES: C=CC1=C(C)c2cc3[nH]c(cc4nc(cc5[nH]c(cc1n2)c(C)c5CCC(=O)O)C(CCC(=O)O)=C4C)c(C)c3C=C] |
| Pyropheophorbide-A | 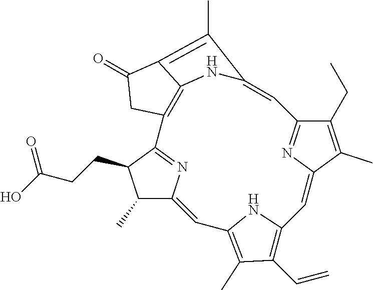 [SMILES: C=Cc1c(C)c2cc3nc(c4c5[nH]c(cc6nc(cc1[nH]2)C(C)=C6CC)c(C)c5C(=O)C4)[C@H](CCC(=O)O)[C@H]3C] |
| Septacidin | 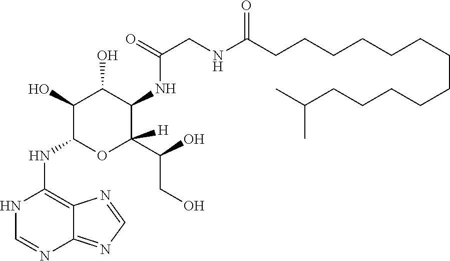 [SMILES: CC(C)CCCCCCCCCCCCC(=O)NCC(=O)N[C@@H]1[C@@H](O)[C@H](O)[C@@H](Nc2[nH]cnc3ncnc2-3)O[C@H]1[C@@H](O)CO] |
| SN-38 | 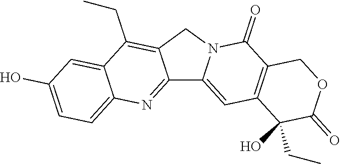 [SMILES: CCc1c2c(nc3ccc(O)cc13)-c1cc3c(c(=O)n1C2)COC(=O)[C@]3(O)CC] |
| Sorafenib | 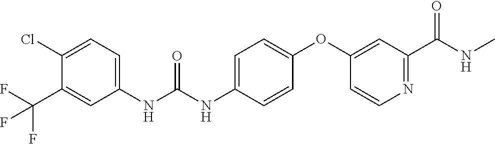 [SMILES: CNC(=O)c1cc(Oc2ccc(NC(=O)Nc3ccc(Cl)c(C(F)(F)F)c3)cc2)ccn1] |
| Streptozocin | 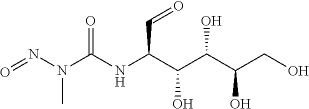 [SMILES: CN(N=O)C(=O)N[C@@H](C=O)[C@@H](O)[C@H](O)[C@H](O)CO] |
| Sunitinib | 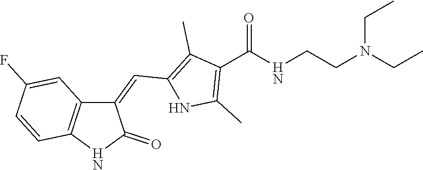 [SMILES: CCN(CC)CCNC(=O)c1c(C)[nH]c(/C=C2\C(=O)Nc3ccc(F)cc32)c1C] |
| Temozolomide | 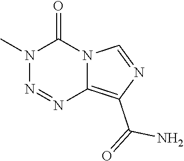 |
| Tipiracil | 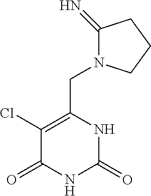 [SMILES: N=C1CCCN1Cc1[nH]c(=O)[nH]c(=O)c1Cl] |
| TPI-287 | 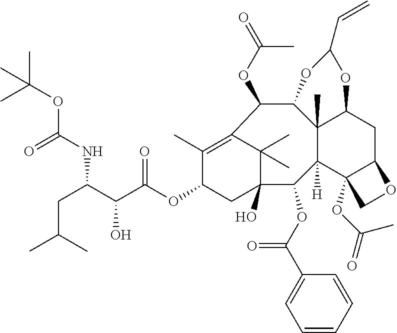 |
| Trifluridine | 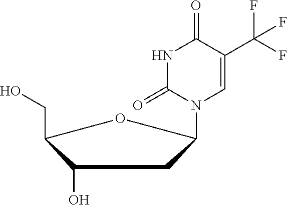 |
| Vadimezan | 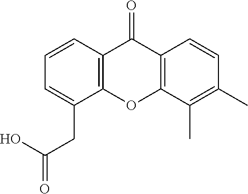 [SMILES: Cc1ccc2c(=O)c3cccc(CC(=O)O)c3oc2c1C] |
| Vemurafenib | 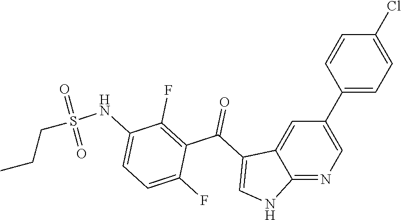 [SMILES: CCCS(=O)(=O)Nc1ccc(F)c(C(=O)c2c[nH]c3ncc(-c4ccc(Cl)cc4)cc23)c1F] |
| Vinblastine | 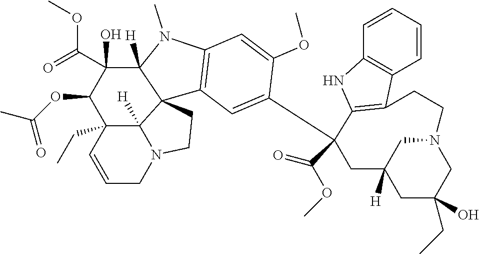 |
| Vincristine | 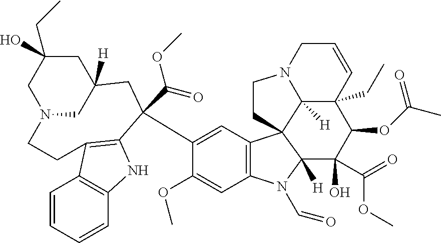 |
| Vinorelbine | 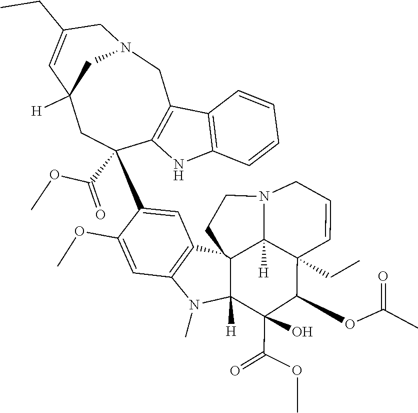 |
| Vipadenant | 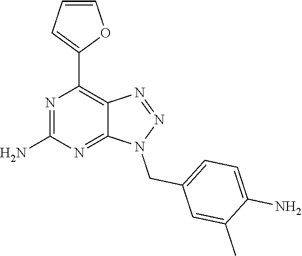 [SMILES: Cc1cc(Cn2nnc3c(-c4ccco4)nc(N)nc32)ccc1N] |
| Vorinostat | 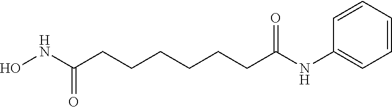 [SMILES: O=C(CCCCCCC(=O)Nc1ccccc1)NO] |

### Methods of Preparation

Provided herein in certain embodiments, are methods of preparing the drug conjugates (e.g., sphingomyelin drug conjugates or DOX-drug conjugates) and/or the nanovesicles (e.g., nanovesicles comprising sphingomyelin drug conjugates and/or nanovesicles comprising sphingomyelin drug conjugates and DOX-drug conjugates) provided herein.

In some embodiments, the present disclosure provides methods for preparing sphingomyelin drug conjugates provided herein. The methods for preparing sphingomyelin drug conjugates can comprise: (a) providing a linker moiety, an anti cancer drug, and a sphingomyelin described herein; (b) conjugating the linker moiety to the anti-cancer drug to form an anti-cancer drug-linker moiety; and (d) conjugating the anti-cancer drug-linker to the sphingomyelin to form the sphingomyelin drug conjugate. In some embodiments, conjugating the anti-cancer drug-linker to the sphingomyelin occurs via a condensation reaction between the anti-cancer drug-linker and the sphingomyelin. In certain of these embodiments, the condensation reaction occurs in the presence of a condensation agent and/or catalyst. Non-limiting examples of condensation agents include: (dimethylamino)-N,N-dimethyl(3H-[1,2,3]triazolo[4,5-b]pyridin-3-yloxy)methaniminium hexafluorophosphate (HATU), 1-ethyl-3(3-dim ethylpropylamine) carbodiimide (EDCI), carbonyldiimidazole (CDI), dicyclohexylcarbodiimide (DCC), 4-nitrophenyl carbonochloridate, and triphosgene Non-limiting examples of catalysts include: 1H-1,2,3-benzotriazole (HOBt), 1-hydroxy-7-azabenzotriazole (HOAt), 4-dimethylaminopyridine (DMAP), 4-pyrrolidinopyridine (4-PPY), and N-hydroxysuccinimide (HOSu).

In some embodiments, the anti-cancer drug comprises camptothecin, paclitaxel, docetaxel, ADU-S100, amrubicin, 5-aminolevulinic acid, AZD4635, BMS-1001, BMS-1166, BMS-200, BMS-202, BMS-242, BMS-242, bortezomib, CA170, cabazitaxel, cabozantinib, canertinib, capecitabine, carboplatin, ceritinib, chlorin e6, cisplatin, dabrafenib, dacarbazine, darolutamide, daunorubicin, degarelix, digoxin, doxorubicin, epacadostat, epirubicin, eribulin, esorubicin, etoposide, fingolimod, 5-fluorouracil, galanthamine, gemcitabine, idarubicin, imatinib, imiquimod, indoximod, irinotecan, ixabepilone, lenvatinib, memantine, methotrexate, mitoxantrone, NIR178, NLG919, oxaliplatin, pazopanib, pemetrexed, preladenant, protoporphyrin IX (PP IX), pyropheophorbide-A, septacidin, SN-38, sorafenib, streptozocin, sunitinib, temozolomide, tipiracil, TPI-287, trifluridine, vadimezan, vemurafenib, vinblastine, vincristine, vinorelbine, vipadenant, vorinostat, or a combination thereof.

In some embodiments, the methods result in the preparation of one or more compound selected from the group consisting of: (1; SM-Ester-CPT), (2; SM-Glycine-CPT), (3; SM-SS-CPT), (4; SM-CSS-CPT), (5; SM-DMM-CPT), (6; SM-SCS-CPT), (7; SM-GFLG-CPT), (8; SM-NN-CPT), (9; SM-PLGLAG-CPT), (10; SM-AANK-CPT), (11; SM-B-SN-38), (12; SM-Ester-PTX), (13; SM-SS-Paclitaxel), (14; SM-CSS-Paclitaxel), (15; SM-Glycine-Paclitaxel), (16; SM-Ester-Docetaxel), (17; SM-SS-Docetaxel), (18; SM-CSS-Docetaxel), (19; SM-Glycine-Docetaxel), (20; SM-Ester-Epacadostat), (21; SM-B-Bortezomib), (22; SM-CSS-Imatinib), (23; SM-CSS-Canertinib), (24; SM-CSS-Ceritinib), (25; SM-CSS-Dabrafenib), (26; SM-CSS-Vemurafenib), (27; SM-Oxo-Oxaliplatin), (28; SM-Ester-Vorinostat), (29; SM-CSS-Preladenant), (30; SM-CSS-BMS-1166), (31; SM-CSS-BMS-1001), (32; SM-CSS-BMS-200), (33; SM-CSS-ADU-S100), (34; SM-SCS-Vadimezan), (35; SM-Ester-Pyropheophorbide A), and (36; SM-Ester-Protoporphyrin IX).

In yet another embodiment, the present disclosure provides methods for preparing DOX-conjugated drug conjugates provided herein. The methods of preparing the DOX-drug conjugate can comprise: (a) providing a linker moiety, an anti cancer drug, and a DOX described herein; and (b) conjugating the linker moiety to the anti-cancer drug to form an anti-cancer drug-linker moiety; and (d) conjugating the anti-cancer drug-linker to the DOX to form the DOX-drug conjugate. In some embodiments, conjugating the anti-cancer drug-linker to the DOX occurs via a condensation reaction between the anti-cancer drug-linker and the DOX. In certain of these embodiments, the condensation reaction occurs in the presence of a condensation agent and/or catalyst. Non-limiting examples of condensation agents include: carbonyldiimidazole (CDI), 4-nitrophenyl carbonochloridate, and triphosgene. Non-limiting examples of catalysts include: dimethylaminopyridine (DMAP), 4-pyrrolidinopyridine (4-PPY), and trifluoroacetic acid (TFA)

In some embodiments, the anti-cancer drug comprises indoximod, bortezomib, epacadostat, imiquimod, imatinib, canertinib, ceritinib, dabrafenib, vemurafenib, vorinostat, ADU-S100, amrubicin, AZD4635, BM S-1001, BMS-1166, BMS-200, BMS202, BMS-242, CA170, cabazitaxel, cabozantinib, camptothecin, capecitabine, carboplatin, cisplatin, dacarbazine, darolutamide, degarelix, digitoxin, digoxin, docetaxel, eribulin, etoposide, 5-fluorouracil, gem citabine, irinotecan, ixabepilone, lenvatinib, methotrexate, mitoxantrone, NIR178, NLG919, oxaliplatin, paclitaxel, pazopanib, pemetrexed, preladenant, septacidin, SN-38, sorafenib, streptozocin, sunitinib, temozolomide, tipiracil, trifluridine, vadimezan, vinblastine, vincristine, vinorelbine, vipadenant, or combinations thereof.

In some embodiments, the methods result in the preparation of one or more compound selected from the group consisting of: (37; Doxorubicin-Hydrazone-SS-Indoximod), (38; Doxorubicin-GFLG-Indoximod), (39; Doxorubicin-DMM-Indoximod), (40; Doxorubicin-AANK-Indoximod), (41; Doxorubicin-Hydrazone-B-Bortezomib), (42; Doxorubicin-Epacadostat), (43; Doxorubicin-SS-Imiquimod), (44; Doxorubicin-SS-Imatinib), (45; Doxorubicin-SS-Canertinib), (46; Doxorubicin-SS-Ceritinib), (47; Doxorubicin-SS-Dabrafenib), (48; Doxorubicin-SS-Vemurafenib), (49; Doxorubicin-Hydrazone-Ester-Vorinostat), (50; Doxorubicin-Oxo-Oxaliplatin), (51; Doxorubicin-SS-Preladenant), (52; Doxorubicin-SS-Vipadenant), (53; Doxorubicin-SS-ADU-S100), and (54; Doxorubicin-SCS-Vadimezan).

In some embodiments, the present disclosure provides methods for preparing a nanovesicle comprising a sphingomyelin-drug conjugate and a DOX-drug conjugate provided herein. The methods can comprise: (a) self-assembling one or more sphingomyelin-drug conjugates into a nanovesicle, (b) incubating the nanovesicle with one or more DOX-drug conjugates, wherein the DOX-drug conjugates enter into an interior core of the nanovesicle to form the nanovesicle comprising the sphingomyelin-drug conjugate and a DOX-drug conjugate. In some embodiments, upon entering the interior core of the nanovesicle, the drug of the DOX-drug conjugate precipitates from the DOX-drug conjugate, releasing both DOX and the drug into the interior core of the nanovesicle.

In some embodiments, the methods comprise self-assembling of the one or more sphingomyelin-drug conjugates into a thin film (e.g., via hydration methods).

In some embodiments, the methods further comprise combining the nanovesicles with a transmembrane agent prior to incubation with the one or more DOX-drug conjugates. In certain of these embodiments, a transmembrane gradient agent is added to the nanovesicles (e.g., via sonication) to form a nanovesicle comprising a transmembrane gradient agent. In some embodiments, the methods include incubating the DOX-drug conjugate with the nanovesicle comprising the transmembrane gradient agent for a period of time and/or temperature, whereby the DOX-drug conjugate enters into an interior core of the nanovesicle. In certain of these embodiments, the period of time is 30 min to 90 minutes and the temperature is 50° C. to 70° C.

In some embodiments, the transmembrane gradient agent comprises one or more of citric acid, triethylammonium sucrose octasulfate (TEA8SOS), ammonium salts, e.g., ammonium sulfate, ammonium α-cyclodextrin sulfate, ammonium sucrose octasulfate, ammonium phosphate, ammonium β-cyclodextrin sulfate, ammonium β-cyclodextrin phosphate, ammonium γ-cyclodextrin sulfate, ammonium γ-cyclodextrin phosphate, ammonium α-cyclodextrin phosphate, ammonium acetate, or ammonium citrate; trimethylammonium salts, e.g., trimethylammonium sucrose octasulfate, trimethylammonium sulfate, trimethylammonium α-cyclodextrin sulfate, trimethylammonium γ-cyclodextrin sulfate, trimethylammonium α-cyclodextrin phosphate, trimethylammonium β-cyclodextrin sulfate, trimethylammonium γ-cyclodextrin phosphate, trimethylammonium phosphate, trimethylammonium β-cyclodextrin phosphate, trimethylammonium citrate, or trimethylammonium acetate; or triethylammonium salts, e.g., triethylammonium sulfate, triethylammonium γ-cyclodextrin sulfate, triethylammonium α-cyclodextrin sulfate, triethylammonium β-cyclodextrin sulfate, triethylammonium phosphate, triethylammonium β-cyclodextrin phosphate, triethylammonium α-cyclodextrin phosphate, triethylammonium γ-cyclodextrin phosphate, triethylammonium acetate, or triethylammonium citrate; or combinations thereof.

In some embodiments, the methods further comprise conjugating the nanovesicle to one or more tumor targeting ligands that can further improve the intratumoral uptake and antitumor efficacy of the nanovesicle. In some embodiments, the one or more tumor targeting ligand comprises one or more small molecules selected from the group consisting for: folate or folic acid, anisamide, phenylboronic acid, glycyrrhizic acid, pamidronic acid, triphenylphosphine, flavin mononucleotide; Polysaccharides: hyaluronic acid, galactose, chitosan, mannose, heparin, dextran, N-acetyl-β-D-galactosamine, sialic acid, lactobionic acid; Proteins: transferrin, EGFP-EGF1, AopB, ApoE, lactoferrin, tumor necrosis factor (TNF)-related apoptosis-inducing ligand (TRAIL); Antibodies: intercellular adhesion molecule 1 antibody (ICAM-1), CD44 antibody, EGFR antibody (cetuximab, panitumumab), PD-L1 antibody, EpCAM antibody, EphA10 antibody, AFP antibody, AMG655 antibody; Peptides: arginine-glycine-aspartate (RGD), asparagine-glycine-arginine (NGR), melittin (Mel), MT peptide, T7 peptide, Cell-penetrating peptides (CPP), Gly-Sar, mitochondrial targeting peptide (pALDH Leader), K237 peptide, YIGSR peptide, poly(histidine-arginine)6 (H6R6), angiopep-2, octreotide, pardaxin, Fragment C of tetanus toxin (TTC); Aptamers: aptamer S6, aptamer GBI-10, aptamer AS1411, aptamer RP, aptamer R8, aptamer AraHH036, aptamer MUC1, aptamer PSMA, aptamer EpCAM, or combinations thereof.

Methods of Treatment and/or Prevention

Aspects of the present disclosure relate to methods of treating and/or preventing cancer in subject in need thereof comprising administering one or more of the drug conjugates (e.g., sphingomyelin drug conjugates) and/or nanovesicles (e.g., nanovesicles comprising sphingomyelin drug conjugates and/or nanovesicles comprising sphingomyelin drug conjugates and DOX-drug conjugates) of the present disclosure.

In some embodiments, the one or more drug conjugates of the present disclosure include one or more sphingomyelin drug conjugates set forth above. In some embodiments, the one or more sphingomyelin drug conjugates comprises a structure consistent with formulas (I)-(VI). In certain of these embodiments, the one or more sphingomyelin drug conjugates is selected from the group consisting of (1; SM-Ester-CPT), (2; SM-Glycine-CPT), (3; SM-SS-CPT), (4; SM-CSS-CPT), (5; SM-DMM-CPT), (6; SM-SCS-CPT), (7; SM-GFLG-CPT), (8; SM-NN-CPT), (9; SM-PLGLAG-CPT), (10; SM-AANK-CPT), (11; SM-B-SN-38), (12; SM-Ester-PTX), (13; SM-SS-Paclitaxel), (14; SM-CSS-Paclitaxel), (15; SM-Glycine-Paclitaxel), (16; SM-Ester-Docetaxel), (17; SM-SS-Docetaxel), (18; SM-CSS-Docetaxel), (19; SM-Glycine-Docetaxel), (20; SM-Ester-Epacadostat), (21; SM-B-Bortezomib), (22; SM-CSS-Imatinib), (23; SM-CSS-Canertinib), (24; SM-CSS-Ceritinib), (25; SM-CSS-Dabrafenib), (26; SM-CSS-Vemurafenib), (27; SM-Oxo-Oxaliplatin), (28; SM-Ester-Vorinostat), (29; SM-CSS-Preladenant), (30; SM-CSS-BMS-1166), (31; SM-CSS-BMS-1001), (32; SM-CSS-BMS-200), (33; SM-CSS-ADU-S100), (34; SM-SCS-Vadimezan), (35; SM-Ester-Pyropheophorbide A), or (36; SM-Ester-Protoporphyrin IX).

In yet another embodiment, the one or more nanovesicles of the present disclosure include one or more nanovesicles comprising a sphingomyelin drug conjugate set forth above. In some embodiments, the one or more nanovesicles comprise a sphingomyelin drug conjugate comprising a structure consistent with formulas (I)-(VI). In some embodiments, the sphingomyelin drug conjugate is selected from the group consisting of (1; SM-Ester-CPT), (2; SM-Glycine-CPT), (3; SM-SS-CPT), (4; SM-CSS-CPT), (5; SM-DMM-CPT), (6; SM-SCS-CPT), (7; SM-GFLG-CPT), (8; SM-NN-CPT), (9; SM-PLGLAG-CPT), (10; SM-AANK-CPT), (11; SM-B-SN-38), (12; SM-Ester-PTX), (13; SM-SS-Paclitaxel), (14; SM-CSS-Paclitaxel), (15; SM-Glycine-Paclitaxel), (16; SM-Ester-Docetaxel), (17; SM-SS-Docetaxel), (18; SM-CSS-Docetaxel), (19; SM-Glycine-Docetaxel), (20; SM-Ester-Epacadostat), (21; SM-B-Bortezomib), (22; SM-CSS-Imatinib), (23; SM-CSS-Canertinib), (24; SM-CSS-Ceritinib), (25; SM-CSS-Dabrafenib), (26; SM-CSS-Vemurafenib), (27; SM-Oxo-Oxaliplatin), (28; SM-Ester-Vorinostat), (29; SM-CSS-Preladenant), (30; SM-CSS-BMS-1166), (31; SM-CSS-BMS-1001), (32; SM-CSS-BMS-200), (33; SM-CSS-ADU-S100), (34; SM-SCS-Vadimezan), (35; SM-Ester-Pyropheophorbide A), or (36; SM-Ester-Protoporphyrin IX).

In another embodiment, the one or more nanovesicles provided herein includes a sphingomyelin drug conjugate and DOX-drug conjugates set forth above. In some embodiments, the sphingomyelin drug conjugates comprise a structure consistent with formulas (I)-(VI). In certain of these embodiments, the DOX-drug conjugates comprise a structure consistent with formula (VII)-(XVII). In certain of these embodiments, the nanovesicle comprises one or more one or more sphingomyelin drug conjugates formulated as nanovesicle selected from the group consisting of: (1; SM-Ester-CPT), (2; SM-Glycine-CPT), (3; SM-SS-CPT), (4; SM-CSS-CPT), (5; SM-DMM-CPT), (6; SM-SCS-CPT), (7; SM-GFLG-CPT), (8; SM-NN-CPT), (9; SM-PLGLAG-CPT), (10; SM-AANK-CPT), (11; SM-B-SN-38), (12; SM-Ester-PTX), (13; SM-SS-Paclitaxel), (14; SM-CSS-Paclitaxel), (15; SM-Glycine-Paclitaxel), (16; SM-Ester-Docetaxel), (17; SM-SS-Docetaxel), (18; SM-CSS-Docetaxel), (19; SM-Glycine-Docetaxel), (20; SM-Ester-Epacadostat), (21; SM-B-Bortezomib), (22; SM-CSS-Imatinib), (23; SM-CSS-Canertinib), (24; SM-CSS-Ceritinib), (25; SM-CSS-Dabrafenib), (26; SM-CSS-Vemurafenib), (27; SM-Oxo-Oxaliplatin), (28; SM-Ester-Vorinostat), (29; SM-CSS-Preladenant), (30; SM-CSS-BMS-1166), (31; SM-CSS-BMS-1001), (32; SM-CSS-BMS-200), (33; SM-CSS-ADU-S100), (34; SM-SCS-Vadimezan), (35; SM-Ester-Pyropheophorbide A), and (36; SM-Ester-Protoporphyrin IX). In certain of these embodiments, the nanovesicle further comprises comprising one or more DOX-drug conjugates incorporated into an interior of the nanovesicles selected from the group consisting of: (37; Doxorubicin-Hydrazone-SS-Indoximod), (38; Doxorubicin-GFLG-Indoximod), (39; Doxorubicin-DMM-Indoximod), (40; Doxorubicin-AANK-Indoximod), (41; Doxorubicin-Hydrazone-B-Bortezomib), (42; Doxorubicin-Epacadostat), (43; Doxorubicin-SS-Imiquimod), (44; Doxorubicin-SS-Imatinib), (45; Doxorubicin-SS-Canertinib), (46; Doxorubicin-SS-Ceritinib), (47; Doxorubicin-SS-Dabrafenib), (48; Doxorubicin-SS-Vemurafenib), (49; Doxorubicin-Hydrazone-Ester-Vorinostat), (50; Doxorubicin-Oxo-Oxaliplatin), (51; Doxorubicin-SS-Preladenant), (52; Doxorubicin-SS-Vipadenant), (53; Doxorubicin-SS-ADU-S100), or (54; Doxorubicin-SCS-Vadimezan). In one aspect, the combination comprises compound (4 SM-CSS-CPT) and compound (37; Doxorubicin-Hydrazone-SS-Indoximod).

In some embodiments, the methods include treating and/or preventing cancer via a combination therapy comprising administering one or more drug conjugates and/or one more nanovesicles described herein with a secondary therapy, such as a radiation therapy, a surgery, an antibody, or any combination thereof. In some embodiments, administration one or more drug conjugates and/or one more nanovesicles in combination with radiation therapy and/or an antibody therapy results in an enhancement of said radiation therapy and/or an antibody therapy such that, for example, a smaller dosage of the radiation and/or antibody therapy may be effective for treatment and/or prevention.

In some of these embodiments, the cancer is adrenal cancer, anal cancer, basal and squamous cell skin cancer, bile duct cancer, bladder cancer, bone cancer, brain and spinal cord tumors (e.g., astrocytoma, glioblastoma multiforme, meningioma), breast cancer, cervical cancer, colorectal cancer, endometrial cancer, esophagus cancer, Ewing family of tumors, eye cancer (ocular melanoma), gallbladder cancer, gastrointestinal neuroendocrine (carcinoid) tumors, gastrointestinal stromal tumor (gist), gestational trophoblastic disease, Kaposi sarcoma, kidney cancer, laryngeal and hypopharyngeal cancer, liver cancer, lung cancer, lung carcinoid tumor, malignant mesothelioma, melanoma skin cancer, Merkle cell skin cancer, nasal cavity and paranasal sinuses cancer, nasopharyngeal cancer, neuroblastoma, non-small cell lung cancer, neoplasm of the central nervous system (CNS), oral cavity and oropharyngeal cancer, osteosarcoma, ovarian cancer, pancreatic cancer, pancreatic neuroendocrine tumor (net), penile cancer, pituitary tumors, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer, uterine sarcoma, vaginal cancer, vulvar cancer, Waldenstrom macroglobulinemia, Wilms tumor, squamous cell cancer, cancers of unknown primary (CUP), environmentally induced cancers, combinations of the cancers, and metastatic lesions of the cancers. In some embodiments, the cancer is leukemia or lymphoma, for example, lymphoblastic lymphoma or B-cell Non-Hodgkin's lymphoma.

In some of these embodiments, the cancer is a hematologic malignancy. In some embodiments, the hematologic malignancy is chronic lymphocytic leukemia (CLL), acute leukemia, acute lymphoid leukemia (ALL), B-cell acute lymphoid leukemia (B-ALL), T-cell acute lymphoid leukemia (T-ALL), T-cell lymphoma, B-cell lymphoma, chronic myelogenous leukemia (CML), acute myelogenous leukemia, B-cell prolymphocytic leukemia, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia. In other embodiments, the cancer is a human hematologic malignancy such as myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplastic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

In some embodiments, the cancer comprises a solid tumor. In some embodiments, the solid tumor is lung cancer, colorectal cancer, breast cancer, pancreatic cancer, gallbladder cancer, brain and spinal cord cancer, head and neck cancer, skin cancers, testicular cancer, prostate cancer, ovarian cancer, renal cell carcinoma (RCC), bladder cancer and hepatocellular carcinoma (HCC).

In some embodiments, the one or more drug conjugates (e.g., sphingomyelin drug conjugates or DOX-drug conjugates) and/or the nanovesicles of present disclosure is present in a composition. In certain embodiments, the composition is a pharmaceutical composition.

In some embodiments, the methods include administering a therapeutically effective amount of one or more drug conjugates (e.g., sphingomyelin drug conjugates or DOX-drug conjugates) and/or the nanovesicles of the present disclosure.

### Kits

Provided herein in certain embodiments are kits comprising one or more drug conjugates (e.g., sphingomyelin drug conjugates or DOX-drug conjugates) and/or the nanovesicles (e.g., nanovesicles comprising sphingomyelin drug conjugates and/or nanovesicles comprising sphingomyelin drug conjugates and DOX-drug conjugates) provided herein. In certain embodiments, the kits further comprise instructions for use.

In some embodiments, the kits provided herein are for use in preparing one or more drug conjugates (e.g., sphingomyelin drug conjugates or DOX-drug conjugates) and/or the nanovesicles (e.g., nanovesicles comprising sphingomyelin drug conjugates and/or nanovesicles comprising sphingomyelin drug conjugates and DOX-drug conjugates) provided herein. In some embodiments, the kits comprises a drug, linker moiety, and/or sphingomyelin, and methods of using the provided components to generate a sphingomyelin drug conjugate. In another embodiment, the kits comprises a drug, linker moiety, and/or DOX, and methods of using the provided components to generate a DOX-drug conjugate. In yet another embodiment, the kit comprises: (a) a drug, linker moiety, and/or sphingomyelin for forming a sphingomyelin drug conjugate and (b) a drug, linker moiety, and/or DOX for forming a DOX-drug conjugate, and methods of using the provided components to generate a nanovesicle provided herein.

In some embodiments, the kits provide herein are for use in a method of treatment and/or prevention of cancer. For example, the kit may comprise one or more drug conjugates (e.g., sphingomyelin drug conjugates or DOX-drug conjugates) and/or the nanovesicles (e.g., nanovesicles comprising sphingomyelin drug conjugates and/or nanovesicles comprising sphingomyelin drug conjugates and DOX-drug conjugates) provided herein, and may further comprise instructions for preparing and/or administering the same.

As can be appreciated from the disclosure above, the present invention has a wide variety of applications. The invention is further illustrated by the following examples, which are only illustrative and are not intended to limit the definition and scope of the invention in any way.

### EXAMPLES

### Example 1—Synthesis of Sphingomyelin Drug Conjugates

The following example provides representative synthetic protocols and associated synthetic reaction schemes for the synthesis of sphingomyelin drug conjugates of the present disclosure.

### Chemical Materials

(S)-(+)-camptothecin (CPT, 98%), 1-methyl-D-tryptophan (IND, 98%), Doxorubicin hydrochloride (DOX, 98%), di(1H-imidazol-1-yl)methanone (CDI, 98%), O-(7-azabenzotriazol-1-yl)-N, N, N,N-tetramethyl uronium hexafluorophosphate (HATU, 98%), 2,3,4,6,7,8,9,10-octahydropyrimido[1,2-a]azepine (DBU, 98%), and (tert-butoxycarbonyl)glycine (98%) were purchased from BLDpharm (Shanghai, China). Sphingomyelin (egg, 99%), DSPE-PEG2K (99%), DSPE-PEG2K-Folate (99%), DSPE-Cy5.5 (99%), and cholesterol (ovine, 99%) were purchased from Avanti (Alabama, USA). Succinic anhydride (98%), N,N-diisopropylethylamine (98%), 4-Dimethylaminopyridine (DMAP, 98%), triphosgene (98%), 4-pyrrolidinopyridine (4-PPY, 98%), and EDCI (98%), di-tert-butyl decarbonate (98%) were purchased from Fisher Scientific (USA). 2,2′-dithiodiethanol (98%) was purchased from Sigma-Aldrich (MO, USA). Doxil® and Onivyde® was acquired from Pharmacy Department, Banner University Medical Center Tucson, AZ. Trypsin-EDTA solution, Triton X-100, and Dulbecco's Modified Eagle's Medium (DMEM), RPMI-1640, fetal bovine serum (FBS), and penicillin-streptomycin solution were all purchased from Gibco (MD, USA). All solvents used for chemical reactions were anhydrous, and the eluting solvents for compound purification were HPLC grade.

### Chemical Syntheses

The NMR spectra were recorded using TMS (0 ppm) as the internal standard on a Varian 400 MHz spectrometer for ¹H NMR and ¹³C NMR. ¹H NMR data were reported as follows: chemical shift, multiplicity (s=singlet, d=doublet, m=multiplet), coupling constant in Hertz (Hz) and hydrogen numbers based on integration intensities. ¹³C NMR chemical shifts are reported in ppm relative to the central peak of TMS (0 ppm) as internal standards. The high-resolution mass spectra (HRMS) were generated using an LTQ Orbitrap Velos mass spectrometer with an ESI source (Thermo Scientific). The low-resolution mass spectra were generated on a LCMS-2020+DUIS-2020 (Shimadzu) instrument with an ESI source. The reactions were followed by thin-layer chromatography (TLC, Silica gel 60 F254, Merck KGaA) on glass-packed precoated silica gel plates and visualized in an iodine chamber or with a UV lamp. Flash column chromatography was performed using silica gel (SiliaFlash®P60, 230-400 mesh) purchased from Silicycle Inc.

### CPT-Based Sphingomyelin (SM) Drug Conjugates

Provided below are the synthetic protocols and proposed synthetic schemes for synthesizing SM drug conjugates featuring CPT as the drug.

### A. Synthesis of SM-Ester-CPT

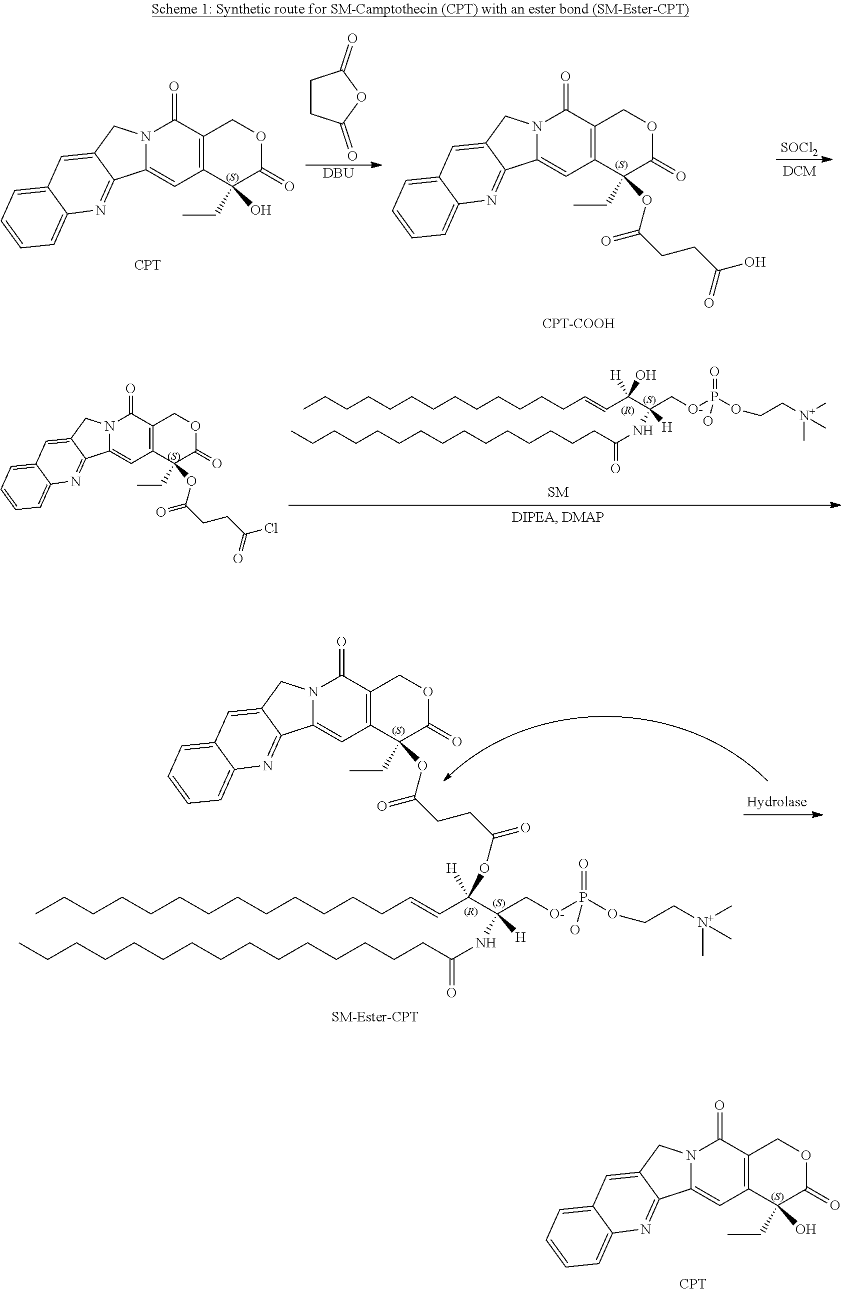

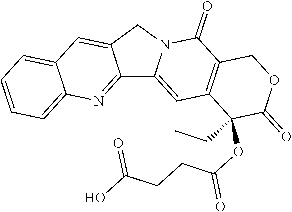 [SMILES: CC[C@@]1(OC(=O)CCC(=O)O)C(=O)OCc2c1cc1n(c2=O)Cc2cc3ccccc3nc2-1]

### Cpt-Cooh

### (S)-4-((4-ethyl-3,14-dioxo-3,4,12,14-tetrahydro-1H-pyrano[3′,4′:6,7] indolizino[1,2-b]quinolin-4-yl)oxy)-4-oxobutanoic acid [51]

1,8-diazabicycloundec-7-ene (DBU, 46 mL, 30 mmol) was added dropwise at 0° C. to a solution of (S)-(+)-camptothecin (3.48 g, 10 mmol) and succinic anhydride (3.0 g, 30 mmol) in 100 mL of anhydrous dichloromethane (DCM). The reaction was further stirred at room temperature for 3 h and monitored by TLC. After the completion of the reaction, the solution was acidified to pH=2.0 by HCl aqueous solution (10%), and then filtrated. The collected pellet was dried under reduced pressure to yellow solid product and used for the next step without further purification. Yellow solid with 96% yield was attained. R_{f}=0.32 (CH₂Cl₂/CH₃OH=20/1). ¹H NMR (400 MHz, DMSO-d₆) δ 8.65 (s, 1H), 8.14 (d, J=8.5 Hz, 1H), 8.09 (d, J=8.1 Hz, 1H), 7.83 (dd, J=11.3, 4.1 Hz, 1H), 7.68 (t, J=7.2 Hz, 1H), 7.09 (s, 1H), 5.53-5.38 (m, 2H), 5.34-5.18 (m, 2H), 2.73 (ddt, J=30.7, 17.8, 6.7 Hz, 2H), 2.12 (dd, J=14.7, 7.2 Hz, 2H), 1.58 (s, 1H), 0.88 (t, J=7.3 Hz, 3H). 13C NMR (101 MHz, DMSO-d₆) δ 173.39, 171.68, 167.58, 156.94, 152.79, 148.30, 146.33, 145.67, 131.95, 130.80, 130.18, 129.42, 128.93, 128.37, 128.11, 119.33, 95.54, 76.30, 66.72, 50.61, 30.82, 28.99, 28.80, 7.96. LC/MS (ESI): 449.1 [M+H]⁺.

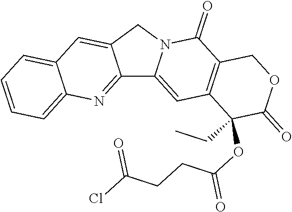 [SMILES: CC[C@@]1(OC(=O)CCC(=O)Cl)C(=O)OCc2c1cc1n(c2=O)Cc2cc3ccccc3nc2-1]

### Cpt-Cocl

### (S)-4-ethyl-3,14-dioxo-3,4,12,14-tetrahydro-1H-pyrano[3′,4′:6, 7]indolizino[1, 2-b]quinolin-4-yl 4-chloro-4-oxobutanoate

Thionyl chloride (3 mL) was added to a solution of intermediate product CPT-COOH (1.0 g, 2.1 mmol) in 20 mL anhydrous DCM at 0° C. The reaction was stirred at room temperature and monitored by the TLC. After the completion of the reaction, the solvent was evaporated under vacuum, the product was used for the next step directly without any further purification.

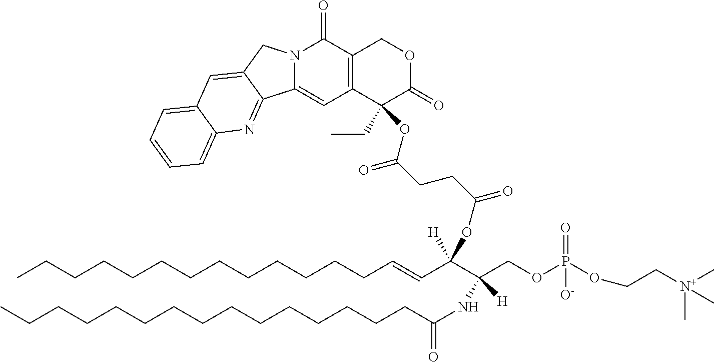 [SMILES: CCCCCCCCCCCCC/C=C/[C@@H](OC(=O)CCC(=O)O[C@]1(CC)C(=O)OCc2c1cc1n(c2=O)Cc2cc3ccccc3nc2-1)[C@H](COP(=O)([O-])OCC[N+](C)(C)C)NC(=O)CCCCCCCCCCCCCCC]

### SM-Ester-CPT

### (2S,3R,E)-3-((4-(((S)-4-ethyl-3,14-dioxo-3,4,12,14-tetrahydro-1H-pyrano[3′,4′: 6,7]indolizino[1,2-b]quinolin-4-yl)oxy)-4-oxobutanoyl)oxy)-2-palmitamidooctadec-4-en-1-yl (2-(trimethylammonio)ethyl) phosphate

N,N-diisopropylethylamine (258.5 mg, 2.0 mmol) and DMAP (12.2 mg, 0.1 mmol) was added to a solution of sphingomyelin (703.0 mg, 1.0 mmol) in anhydrous DCM (30 mL). The mixture solution was stirred at 0° C. with a dropwise addition of a solution of CPT-COCl (932.2 mg, 2.0 mmol) in 10 mL anhydrous DCM. The reaction was stirred at room temperature for 48 h and monitored by TLC. The solvent was then removed under reduced pressure, the product was extracted by DCM (50 mL×5). The organic phase was washed with saturated brine, dried with anhydrous Na₂SO₄, and the solvent was evaporated using rotary evaporator (RV 10 digital, IKA®) under vacuum followed by purification by silica gel flash chromatography with CHCl₃/EtOH/H₂O (v/v/v, 300/200/36) as the eluting solvent. Yellow solid with 42% yield was obtained. R_{f}=0.28 (CHCl₃/EtOH/H₂O=300/200/36). ¹H NMR (400 MHz, CDCl₃) δ 8.36 (s, 1H), 8.19 (d, J=8.4 Hz, 1H), 7.89 (d, J=7.9 Hz, 1H), 7.83-7.78 (m, 1H), 7.65-7.60 (m, 1H), 7.53 (s, 1H), 7.14 (s, 1H), 5.62 (d, J=16.7 Hz, 2H), 5.37-5.24 (m, 4H), 4.24 (s, 2H), 4.17 (s, 1H), 3.83 (s, 2H), 3.68 (d, J=17.9 Hz, 3H), 3.27 (s, 9H), 2.92-2.85 (m, 1H), 2.76-2.69 (m, 1H), 2.61 (dd, J=14.7, 3.3 Hz, 1H), 2.51-2.43 (m, 1H), 2.23 (dd, J=13.8, 7.3 Hz, 1H), 2.15-2.10 (m, 1H), 2.05 (s, 2H), 1.86 (d, J=5.1 Hz, 2H), 1.46 (s, 2H), 1.20 (d, J=11.3 Hz, 46H), 0.94 (t, J=7.1 Hz, 3H), 0.84 J=5.9 Hz, 6H). ¹³C NMR (101 MHz, CDCl₃) δ 173.12, 171.61, 170.78, 167.70, 157.15, 152.13, 148.76, 146.58, 145.43, 137.64, 131.25, 130.66, 129.59, 128.55, 128.17, 128.01, 125.03, 119.82, 95.54, 76.06, 74.21, 67.10, 66.28, 66.26, 63.85, 63.81, 59.31, 59.27, 54.50, 50.90, 50.04, 36.66, 32.23, 31.89, 31.68, 29.70, 29.64, 29.59, 29.54, 29.50, 29.40, 29.33, 28.91, 28.68, 28.30, 25.78, 22.65, 14.09, 7.54. HRMS (ESI) m/z [M+Na]⁺ for C₆₃H₉₇N₄O₁₂P calculated 1155.6733, found 1155.6772; HPLC purity: 95.6%, retention time: 10.260 min.

### B. Synthesis of SM-SS-CPT

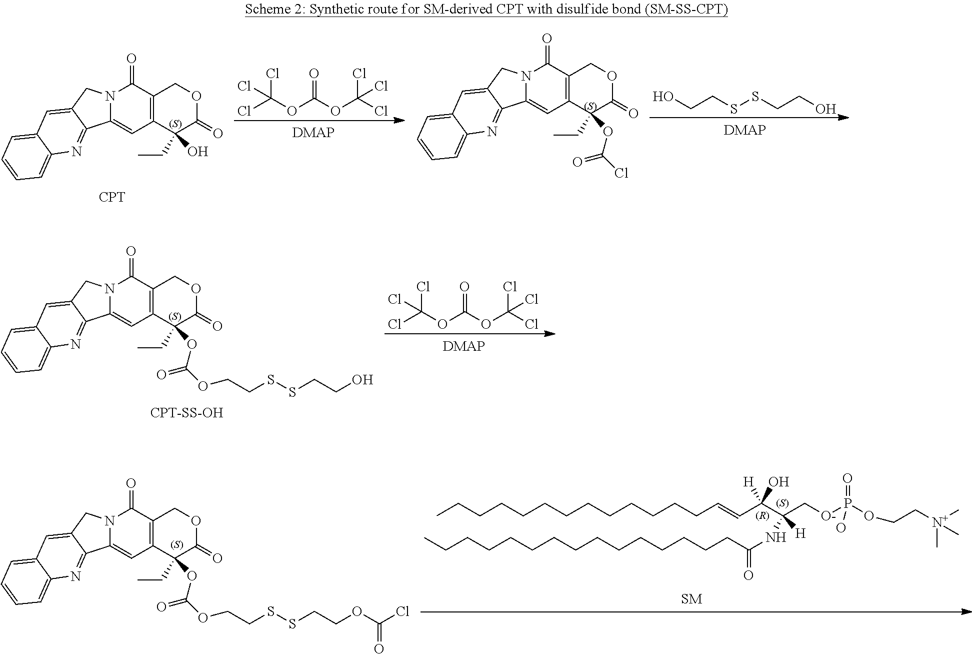

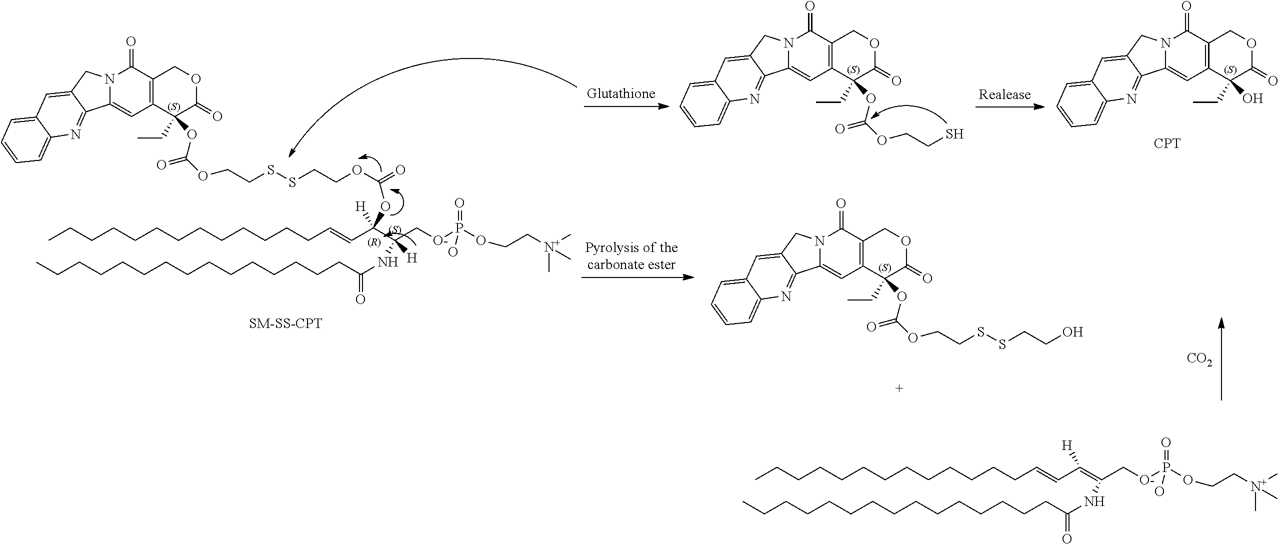

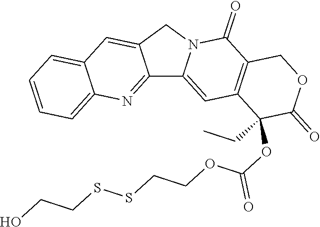 [SMILES: CC[C@@]1(OC(=O)OCCSSCCO)C(=O)OCc2c1cc1n(c2=O)Cc2cc3ccccc3nc2-1]

### Cpt-Ss-Oh

### (S)-4-ethyl-3,14-dioxo-3,4,12,14-tetrahydro-1H-pyrano[3′,4′:6, 7]indolizino[1, 2-b]quinolin-4-yl (2-((2-hydroxyethyl)disulfanyl)ethyl) carbonate [52]

DMAP (3.67 g, 30 mmol, in 15 mL anhydrous DCM) was added dropwise to a solution of (S)-(+)-cam ptothecin (3.48 g, 10 mmol) and triphosgene (1.03 g, 3.4 mmol) in anhydrous DCM (150 mL). The reaction was stirred at room temperature for 30 min, then a solution of 2,2′-dithiodiethanol (9.25 g, 60 mmol) in anhydrous THE (25 mL) was added into the mixture solution. The reaction was further stirred at room temperature for 12 h and monitored by TLC. After completion of the reaction, the mixture solution was washed with 50 mM HCl aqueous solution to remove the DMAP, and then with saturated brine. The organic layer was dried with anhydrous Na₂SO₄, the solvent was evaporated using rotary evaporator under vacuum, and the residue was purified by silica gel flash chromatography. Yellow solid with 71% yield was acquired. R_{f}=0.38 (CH₂Cl₂/CH₃OH=20/1). ¹H NMR (400 MHz, CDCl₃) δ 8.41 (s, 1H), 8.20 (t, J=11.2 Hz, 1H), 7.94 (d, J=8.1 Hz, 1H), 7.84 (t, J=7.2 Hz, 1H), 7.67 (t, J=7.1 Hz, 1H), 7.42 (s, 1H), 5.70 (d, J=17.3 Hz, 1H), 5.38 (d, J=17.3 Hz, 1H), 5.29 (s, 2H), 4.45-4.25 (m, 2H), 3.89 (pd, J=11.8, 6.2 Hz, 2H), 3.27 (t, J=5.9 Hz, 1H), 3.05-2.78 (m, 4H), 2.28 (td, J=14.9, 7.5 Hz, 1H), 2.15 (td, J=15.0, 7.6 Hz, 1H), 1.00 J=7.5 Hz, 3H). ¹³C NMR (101 MHz, DMSO-d₆) δ 167.47, 156.90, 153.21, 152.62, 148.29, 146.66, 145.14, 132.02, 130.86, 130.20, 129.41, 128.94, 128.43, 128.17, 119.60, 94.79, 78.30, 66.87, 66.73, 59.71, 50.75, 41.53, 36.60, 30.74, 7.99. LC/MS (ESI): 529.1 [M+H]⁺.

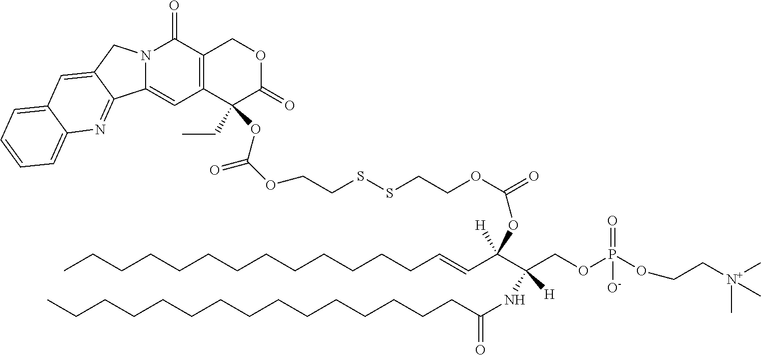 [SMILES: CCCCCCCCCCCCC/C=C/[C@@H](OC(=O)OCCSSCCOC(=O)O[C@]1(CC)C(=O)OCc2c1cc1n(c2=O)Cc2cc3ccccc3nc2-1)[C@H](COP(=O)([O-])OCC[N+](C)(C)C)NC(=O)CCCCCCCCCCCCCCC]

### Sm-Ss-Cpt

### (12R,13S)-1-(((S)-4-ethyl-3,14-dioxo-3, 4,12,14-tetrahydro-1H-pyrano[3′,4′:6,7]indolizino[1,2-b]quinolin-4-yl)oxy)-1,10-dioxo-β-palmitamido-12-((E)-pentadec-1-en-1-yl)-2,9,11-trioxa-5,6-dithiatetradecan-14-yl (2-(trimethylammonio)ethyl) Phosphate

DMAP (367 mg, 3 mmol, in 5 mL anhydrous DCM)was added dropwise to a solution of CPT-SS-OH (528 mg, 1.0 mmol) and triphosgene (103 mg, 0.34 mmol) in anhydrous DCM (100 mL). The reaction mixture was stirred at room temperature for 20 min. Then sphingomyelin (703.0 mg, 1.0 mmol) was added into the mixture solution. The reaction was further stirred at room temperature for 12 h and monitored by TLC. After completion of the reaction, the mixture solution was washed with 50 mM HCl aqueous solution to remove the DMAP, and then with saturated brine. The organic layer was dried with anhydrous Na₂SO₄, the solvent was evaporated using rotary evaporator under vacuum, and the residue was purified by silica gel flash chromatography with CHCl₃/EtOH/H₂O (v/v/v, 300/200/36) as the eluting solvent. Yellow solid with 56% yield was obtained. R_{f}=0.31 (CHCl₃/EtOH/H₂O=300/200/36). ¹H NMR (400 MHz, CDCl₃) δ 8.43 (s, 1H), 8.22 (d, J=8.5 Hz, 1H), 7.95 (d, J=8.1 Hz, 1H), 7.87-7.82 (m, 1H), 7.70-7.65 (m, 1H), 7.61 (d, J=8.3 Hz, 1H), 7.32 (s, 1H), 5.76 (dd, J=14.8, 7.6 Hz, 1H), 5.70 (d, J=17.2 Hz, 1H), 5.48-5.42 (m, 1H), 5.39 (d, J=17.1 Hz, 1H), 5.31 (s, 2H), 5.15 (t, J=8.1 Hz, 1H), 4.45-4.31 (m, 4H), 4.29-4.23 (m, 2H), 3.97 (d, J=5.6 Hz, 2H), 3.83 (dt, J=35.0, 13.3 Hz, 3H), 3.35 (s, 9H), 2.95 (t, J=6.2 Hz, 2H), 2.88 (dd, J=7.2, 5.5 Hz, 2H), 2.31 (s, 2H), 2.17-2.12 (m, 2H), 2.01-1.93 (m, 2H), 1.54 (s, 2H), 1.25 (t, J=12.5 Hz, 46H), 1.01 (t, J=7.4 Hz, 3H), 0.87 (t, J=6.7 Hz, 6H). ¹³C NMR (101 MHz, CDCl₃) δ 173.33, 167.51, 157.19, 153.94, 153.44, 152.17, 148.85, 146.63, 145.47, 138.23, 131.24, 130.72, 129.63, 128.47, 128.19, 128.10, 124.38, 119.94, 95.82, 78.12, 78.06, 77.18, 67.09, 66.51, 66.36, 65.21, 63.76, 59.25, 54.65, 51.27, 50.07, 37.06, 36.77, 36.71, 32.31, 31.89, 31.76, 29.71, 29.70, 29.65, 29.60, 29.56, 29.51, 29.40, 29.34, 28.91, 25.79, 22.66, 14.09, 7.64. HRMS (ESI) m/z [M+H]⁺ for C₆₅H₁₀₂N₄O₁₄PS₂ calculated 1257.6566, found 1257.6594; HPLC purity: 95.6%, retention time: 13.371 min.

### C. Synthesis of SM-Glycine-CPT

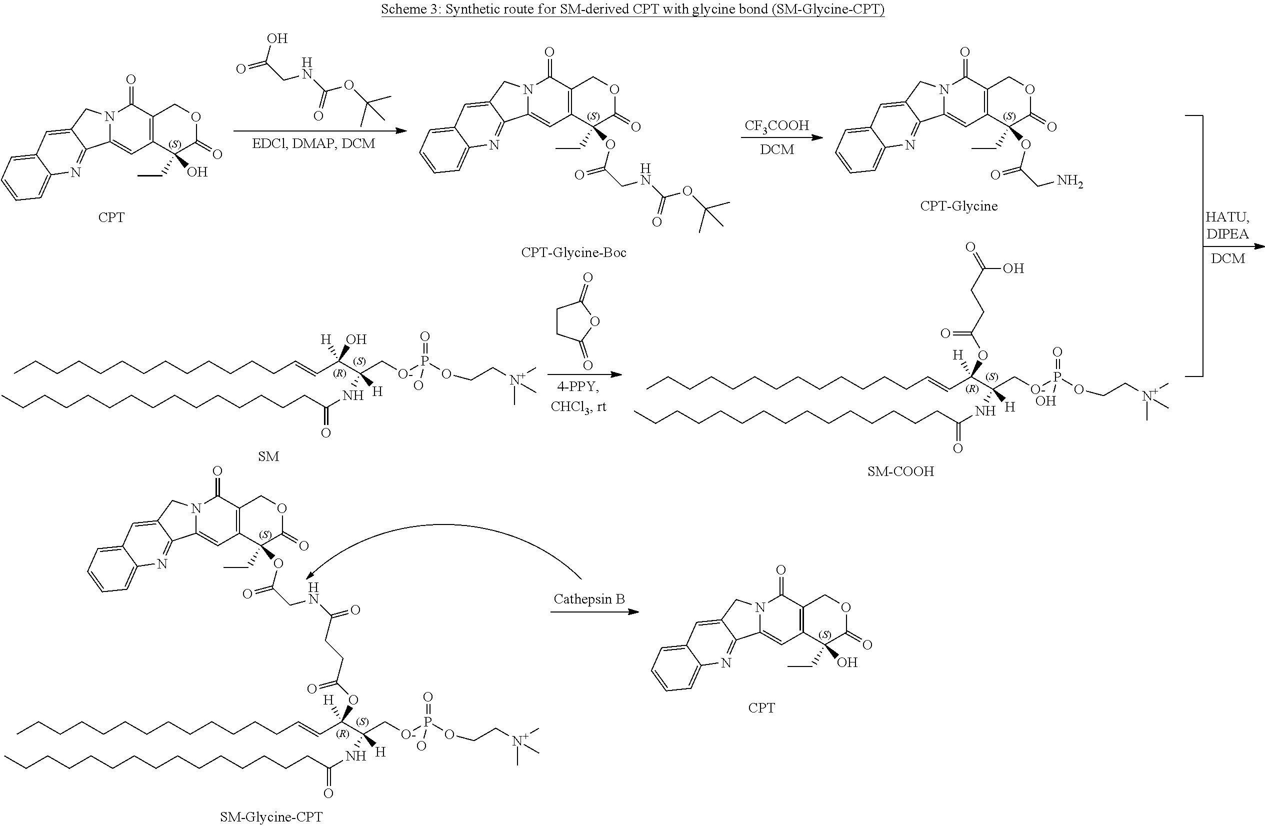

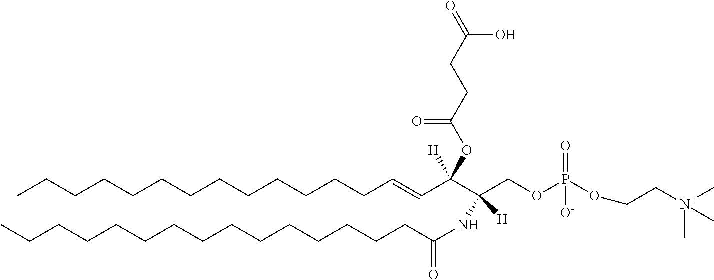 [SMILES: CCCCCCCCCCCCC/C=C/[C@@H](OC(=O)CCC(=O)O)[C@H](COP(=O)([O-])OCC[N+](C)(C)C)NC(=O)CCCCCCCCCCCCCCC]

### Sm-Cooh

### (2S,3R,E)-3-((3-carboxypropanoyl)oxy)-2-palmitamidooctadec-4-en-1-yl (2-(trim ethylammonio)ethyl) Phosphate

4-pyrrolidinopyridine (4-PPY, 44.4 mg, 0.3 mmol) was added to a solution of sphingomyelin (2.1 g, 3.0 mmol) and succinic anhydride (3 g, 30 mmol) in anhydrous CHCl₃ (100 mL). The solution was stirred at room temperature for 12 h and monitored by TLC. After completion of the reaction, the solvent was evaporated using rotary evaporator under vacuum, and the residue was purified by silica gel flash chromatography with CHCl₃/EtOH/H₂O (v/v/v, 300/200/36) as the elution solvent. White solid with 93% yield was garnered. R_{f}=0.23 (CHCl₃/EtOH/H₂O=300/200/36). ¹H NMR (400 MHz, CDCl₃) δ 6.98 (d, J=6.7 Hz, 1H), 5.75-5.68 (m, 1H), 5.39 (dd, J=15.0, 8.3 Hz, 1H), 5.28 (t, J=8.8 Hz, 1H), 4.29 (d, J=4.4 Hz, 3H), 3.94 (s, 2H), 3.77 (s, 2H), 3.28 (s, 9H), 2.64 (dd, J=13.1, 6.3 Hz, 2H), 2.39 (dd, J=14.3, 6.6 Hz, 2H), 2.12 (q, J=13.9 Hz, 2H), 1.97 (d, J=6.6 Hz, 2H), 1.55 (s, 2H), 1.28 (d, J=19.0 Hz, 46H), 0.88 (t, J=6.7 Hz, 6H). ¹³C NMR (101 MHz, CDCl₃) δ 174.74, 173.10, 172.06, 137.75, 125.20, 73.28, 65.76, 65.70, 64.26, 64.23, 59.20, 59.15, 54.32, 50.66, 50.61, 36.66, 32.25, 31.88, 29.74, 29.71, 29.70, 29.69, 29.63, 29.60, 29.56, 29.50, 29.46, 29.33, 28.92, 25.79, 22.64, 14.06. HRMS (ESI) m/z [M+H]⁺ for C₄₃H₈₄N₂O₉P calculated 803.5909, found 803.5928.

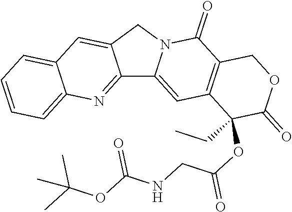 [SMILES: CC[C@@]1(OC(=O)CNC(=O)OC(C)(C)C)C(=O)OCc2c1cc1n(c2=O)Cc2cc3ccccc3nc2-1]

### CPT-Glycine-Boc (S)-4-ethyl-3,14-dioxo-3,4,12,14-tetrahydro-1H-pyrano[3′,4′:6, 7]indolizino[1, 2-b]quinolin-4-yl (tert-butoxycarbonyl)glycinate (CPT-Glycine-Boc)

EDCI (2.10 g, 11.0 mmol) and 2 mL DIPEA was added to a solution of (tert-butoxycarbonyl)glycine (1.75 g, 10.0 mmol) in 100 mL anhydrous DCM followed by stirring at room temperature for 30 min. (S)-(+)-camptothecin (3.48 g, 10 mmol) and DMAP (122 mg, 1.0 mmol) was then added into the mixture solution. The reaction was further stirred at room temperature for 12 h and monitored by TLC. After completion of the reaction, the mixture solution was washed with 50 mM HCl aqueous solution to remove the DMAP, and then with saturated brine. The organic layer was dried with anhydrous Na₂SO₄, the solvent was evaporated using rotary evaporator under vacuum, and the residue was purified by silica gel flash chromatography. Yellow solid with 96% yield was gained. R_{f}=0.43 (petroleum/EtOAc=1/1). ¹H NMR (400 MHz, CDCl₃) δ 8.39 (s, 1H), 8.24 (d, J=8.2 Hz, 1H), 7.94 (d, J=8.1 Hz, 1H), 7.84 (t, J=7.5 Hz, 1H), 7.67 (t, J=7.4 Hz, 1H), 7.27 (d, J=6.1 Hz, 1H), 5.69 (d, J=17.2 Hz, 1H), 5.41 (d, J=17.2 Hz, 1H), 5.29 (d, J=6.3 Hz, 2H), 4.97 (s, 1H), 4.20 (dd, J=18.2, 5.9 Hz, 1H), 4.07 (dd, J=18.1, 4.7 Hz, 1H), 2.30 (td, J=14.8, 7.4 Hz, 1H), 2.17 (td, J=14.8, 7.4 Hz, 1H), 1.77 (s, 1H), 1.53-1.24 (m, 9H), 0.99 (t, J=7.4 Hz, 3H). ¹³C NMR (101 MHz, CDCl₃) δ 169.49, 167.13, 157.28, 155.42, 152.22, 148.86, 146.40, 145.39, 131.05, 130.58, 129.73, 128.35, 128.12, 127.99, 120.10, 96.17, 80.13, 77.18, 76.75, 67.09, 49.93, 42.39, 31.75, 28.22, 7.53. LC/MS (ESI): 506.1 [M+H]⁺.

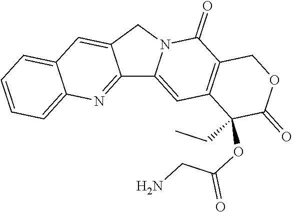 [SMILES: CC[C@@]1(OC(=O)CN)C(=O)OCc2c1cc1n(c2=O)Cc2cc3ccccc3nc2-1]

### CPT-Glycine

### (S)-4-ethyl-3,14-dioxo-3,4,12,14-tetrahydro-1H-pyrano[3′,4′:6, 7]indolizino[1, 2-b]quinolin-4-yl glycinate

CF₃COOH (1 mL)was added to a solution of CPT-Glycine-Boc (505 mg, 1.0 mmol) in anhydrous DCM (50 mL) in an ice bath. The reaction was stirred at room temperature for 0.5 h and monitored by TLC. After completion of the reaction, the solvent was evaporated using rotary evaporator under vacuum. This intermediate was used for the next step immediately without further purification.

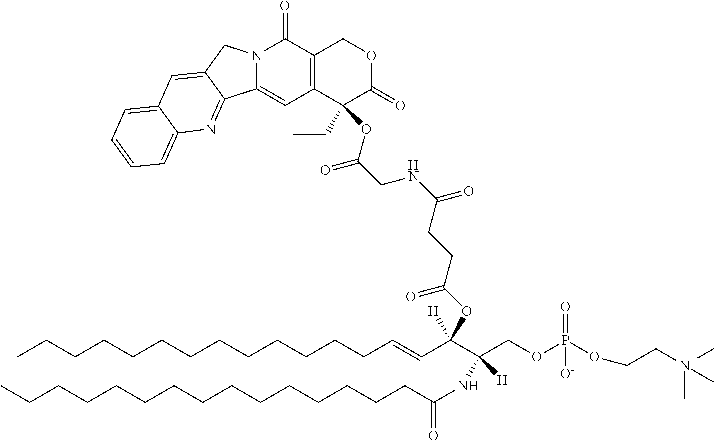 [SMILES: CCCCCCCCCCCCC/C=C/[C@@H](OC(=O)CCC(=O)NCC(=O)O[C@]1(CC)C(=O)OCc2c1cc1n(c2=O)Cc2cc3ccccc3nc2-1)[C@H](COP(=O)([O-])OCC[N+](C)(C)C)NC(=O)CCCCCCCCCCCCCCC]

### SM-Glycine-CPT

### (2S,3R,E)-3-((4-((2-(((S)-4-ethyl-3,14-dioxo-3, 4,12,14-tetrahydro-1H-pyrano[3′,4′:6,7]indolizino[1,2-b]quinolin-4-yl)oxy)-2-oxoethyl)amino)-4-oxobutanoyl)oxy)-2-palmitamidooctadec-4-en-1-yl (2-(trimethylammonio)ethyl) phosphate

DIPEA (2 mL) was added to a solution of SM-COOH (803.0 mg, 1.0 mmol) and HATU (380 mg, 1.0 mmol) in anhydrous DCM (50 mL). The reaction mixture was stirred at room temperature for 30 min. A solution of CPT-Glycine (1.0 mmol) in 10 mL anhydrous DCM was added into the reaction and further stirred for 12 h. After completion of the reaction, the reaction mixture was washed with 50 mM HCl aqueous solution, and then with saturated brine. The organic layer was dried with anhydrous Na₂SO₄, the solvent was removed using rotary evaporator under vacuum, and the residue was purified by silica gel flash chromatography with CHCl₃/EtOH/H₂O (v/v/v, 300/200/36) as the eluting solvent. Pale yellow solid with 78% yield was achieved. R_{f}=0.32 (CHCl₃/EtOH/H₂O=300/200/36). ¹H NMR (400 MHz, CDCl₃) δ 8.60 (s, 1H), 8.36 (s, 1H), 8.21 (d, J=8.5 Hz, 1H), 7.90 (d, J=8.1 Hz, 1H), 7.82-7.77 (m, 1H), 7.66-7.60 (m, 1H), 7.29 (s, 1H), 7.14 (d, J=8.1 Hz, 1H), 5.69-5.59 (m, 2H), 5.39-5.19 (m, 5H), 4.36 (dd, J=18.0, 5.7 Hz, 1H), 4.23 (s, 2H), 4.13 (s, 1H), 4.01 (dd, J=17.9, 4.3 Hz, 1H), 3.86-3.76 (m, 2H), 3.67 (s, 2H), 3.25 (s, 9H), 2.56 (s, 4H), 2.24 (dd, J=14.0, 7.3 Hz, 1H), 2.17-2.13 (m, 1H), 2.12-2.05 (m, 2H), 1.88 (d, J=6.4 Hz, 2H), 1.50 (s, 2H), 1.21 (d, J=10.2 Hz, 46H), 0.93 (t, J=7.3 Hz, 3H), 0.86 (t, J=6.6 Hz, 6H). ¹³C NMR (101 MHz, CDCl₃) δ 173.31, 172.53, 171.97, 169.91, 167.85, 157.16, 152.01, 148.69, 146.52, 145.38, 137.06, 131.19, 130.56, 129.69, 128.42, 128.10, 127.98, 124.65, 119.54, 96.31, 76.46, 73.54, 67.15, 66.28, 66.21, 64.17, 59.25, 59.20, 54.29, 51.11, 51.06, 50.02, 41.00, 36.57, 32.30, 31.90, 31.62, 30.38, 29.93, 29.72, 29.65, 29.61, 29.56, 29.52, 29.39, 29.35, 28.93, 25.86, 22.66, 14.10, 7.53. HRMS (ESI) m/z [M+H]⁺ for C₆₅H₁₀₁N₅O₁₃P calculated 1190.7128, found 1190.7169; HPLC purity: 96.8%, retention time: 10.801 min.

### D. Synthesis of SM-CSS-CPT

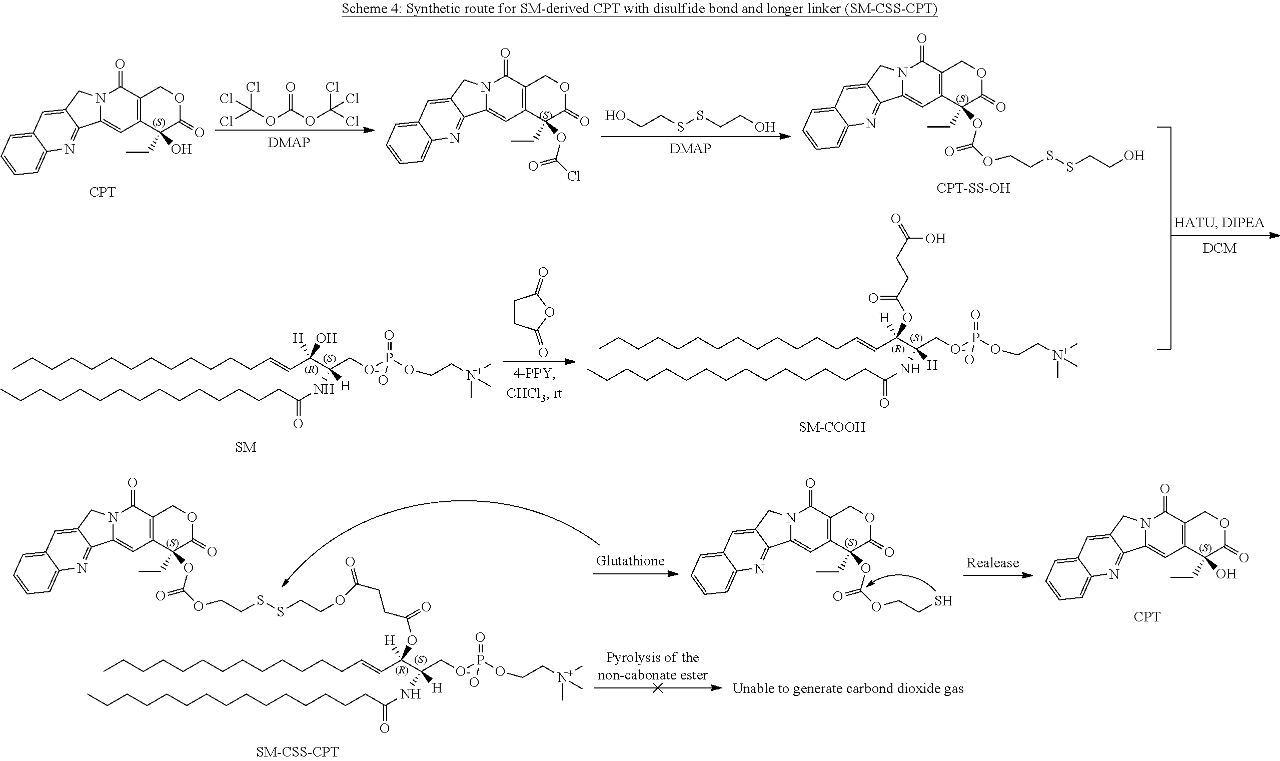

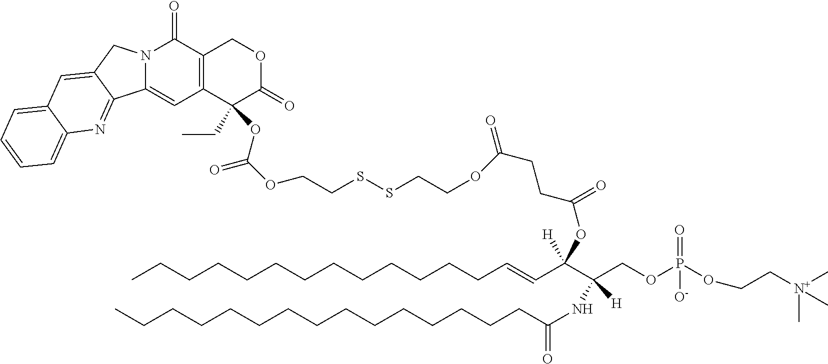 [SMILES: CCCCCCCCCCCCC/C=C/[C@@H](OC(=O)CCC(=O)OCCSSCCOC(=O)O[C@]1(CC)C(=O)OCc2c1cc1n(c2=O)Cc2cc3ccccc3nc2-1)[C@H](COP(=O)([O-])OCC[N+](C)(C)C)NC(=O)CCCCCCCCCCCCCCC]

### Sm-Css-Cpt

### (15R,16S)-1-(((S)-4-ethyl-3,14-dioxo-3, 4,12,14-tetrahydro-1H-pyrano[3′,4′:6,7]indolizino[1,2-b]quinolin-4-yl)oxy)-1,10,13-trioxo-16-palmitamido-15-((E)-pentadec-1-en-1-yl)-2,9,14-trioxa-5,6-dithiaheptadecan-17-yl (2-(trimethylammonio)ethyl) Phosphate

DIPEA (2 mL) was added to a solution of SM-COOH (803.0 mg, 1.0 mmol) and HATU (380 mg, 1.0 mmol) in anhydrous DCM (50 mL). The solution mixture was stirred at room temperature for 30 min. A solution of CPT-SS-OH (528 mg, 1.0 mmol) in 10 mL anhydrous DCM was then added into the reaction mixture and further stirred for 12 h. After completion of the reaction, the reaction mixture was washed with 50 mM HCl aqueous solution and then with saturated brine. The organic layer was dried with anhydrous Na₂SO₄, the solvent was evaporated using rotary evaporator under vacuum, and the residue was purified by silica gel flash chromatography with CHCl₃/EtOH/H₂O (v/v/v, 300/200/36) as the eluting solvent. Pale yellow solid with 78% yield was attained. R_{f}=0.30 (CHCl₃/EtOH/H₂O=300/200/36). ¹H NMR (400 MHz, CDCl₃) δ 8.43 (s, 1H), 8.22 (d, J=8.5 Hz, 1H), 7.96 (d, J=8.1 Hz, 1H), 7.85 (t, J=7.6 Hz, 1H), 7.68 (t, J=7.5 Hz, 1H), 7.39 (d, J=8.8 Hz, 1H), 7.32 (s, 1H), 5.74-5.67 (m, 2H), 5.44-5.31 (m, 5H), 4.38 (dt, J=11.7, 4.9 Hz, 4H), 4.30-4.22 (m, 3H), 4.01-3.87 (m, 2H), 3.85-3.71 (m, 2H), 3.34 (s, 9H), 2.94 (t, J=6.4 Hz, 2H), 2.87 (t, J=6.3 Hz, 2H), 2.62-2.51 (m, 4H), 2.36-2.09 (m, 3H), 1.96 (d, J=6.9 Hz, 3H), 1.54 (s, 2H), 1.24 (d, J=3.9 Hz, 46H), 1.01 (t, J=7.4 Hz, 3H), 0.87 (t, J=6.6 Hz, 6H). ¹³C NMR (101 MHz, CDCl₃) δ 173.24, 172.16, 171.17, 167.37, 157.21, 153.43, 152.21, 148.85, 146.57, 145.49, 137.45, 131.25, 130.72, 129.61, 128.48, 128.22, 128.19, 128.10, 124.81, 120.05, 95.82, 78.05, 77.18, 74.18, 67.07, 66.59, 66.53, 66.46, 63.90, 63.86, 62.36, 59.25, 59.20, 54.66, 51.17, 51.13, 50.05, 37.08, 36.81, 36.65, 32.30, 31.89, 31.81, 29.70, 29.64, 29.59, 29.52, 29.41, 29.34, 28.98, 28.88, 25.82, 22.66, 14.09, 7.63. HRMS (ESI) m/z [M+H]⁺ for C₆₈H₁₀₆N₄O₁₅PS₂ calculated 1313.6828, found 1313.6872; HPLC purity: 97.2%, retention time: 13.074 min.

### E. Synthetic Scheme for the Synthesis of SM-DMM-CPT

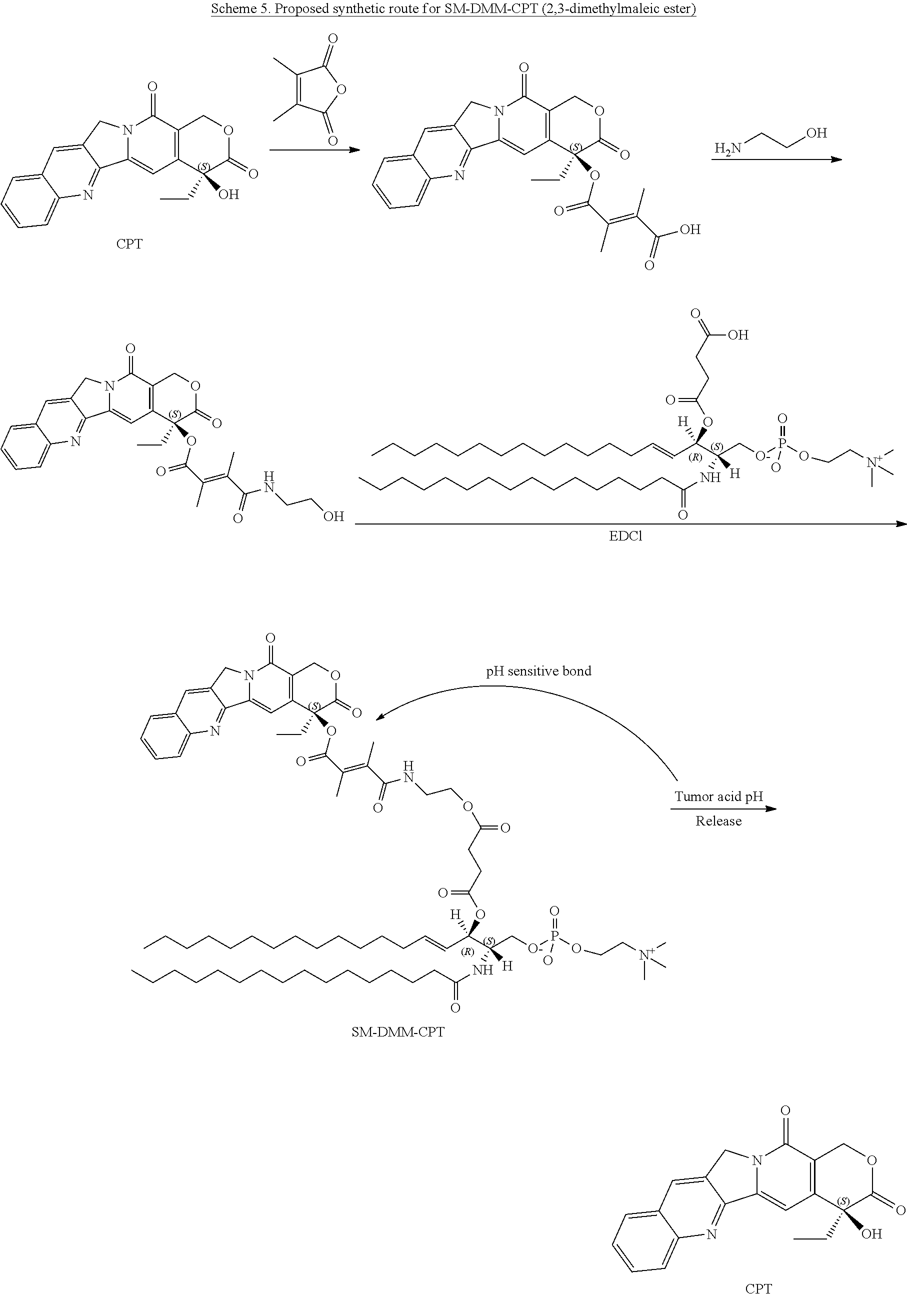

### F. Synthetic Scheme for the Synthesis of SM-SCS-CPT

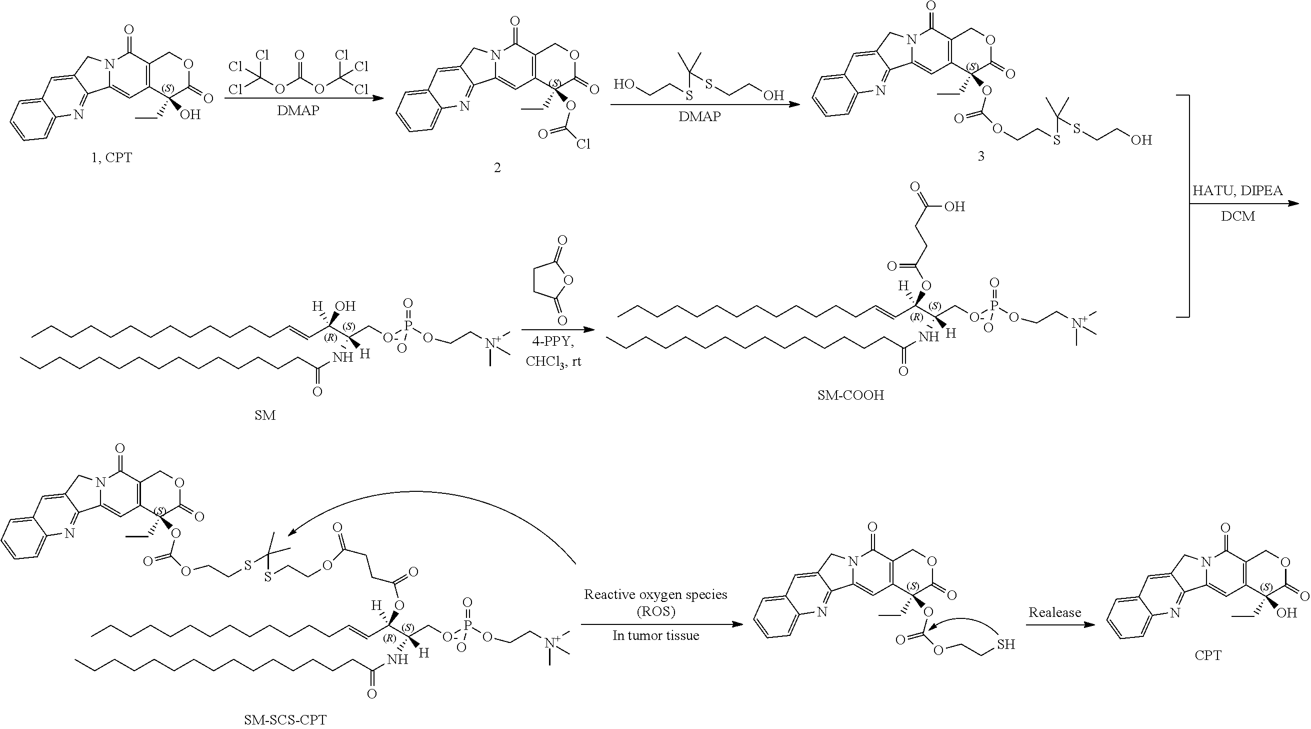

### G. Synthetic Scheme for the Synthesis of SM-GFLG-CPT (Gly-Phe-Leu-Gly)

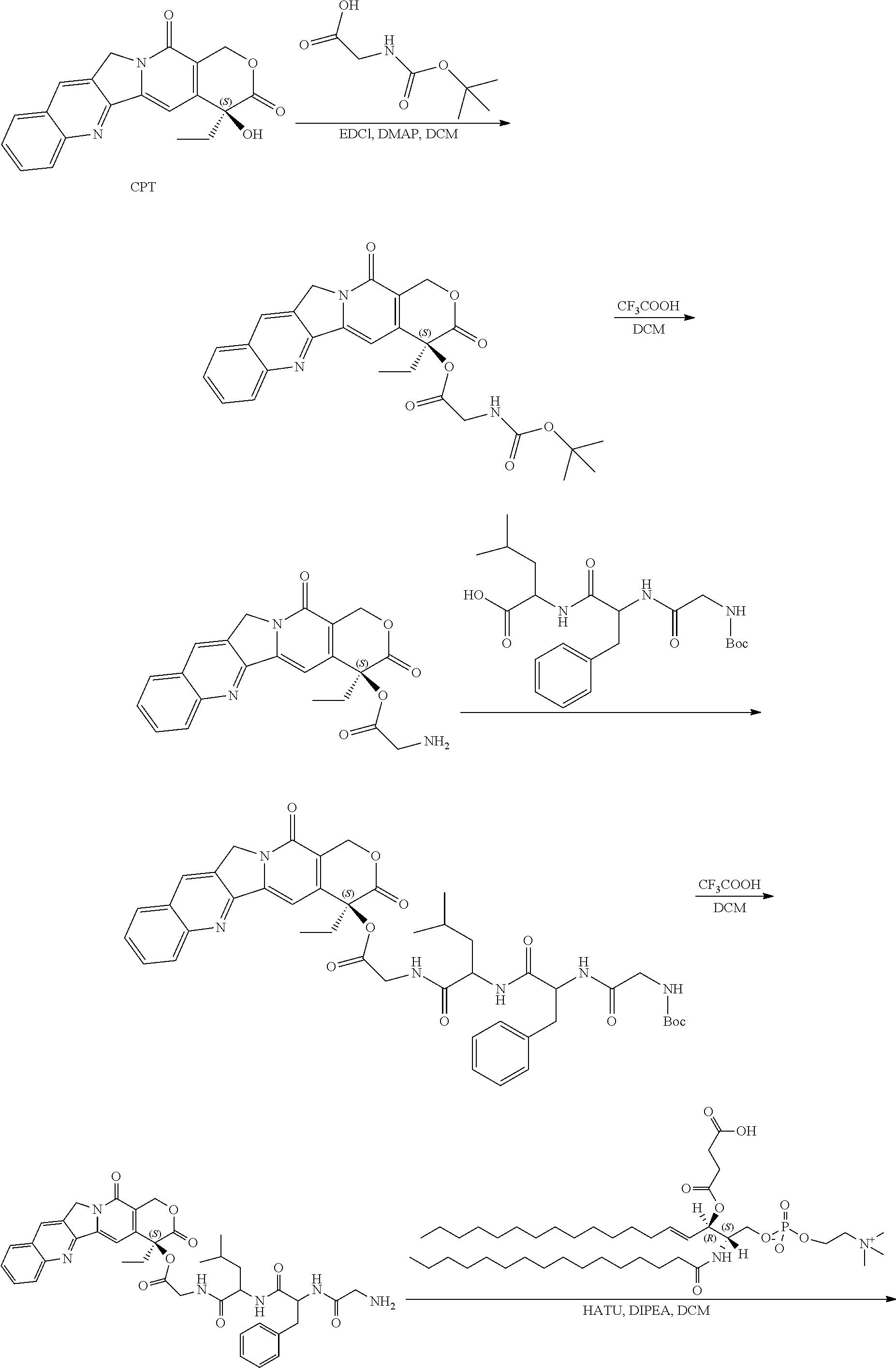

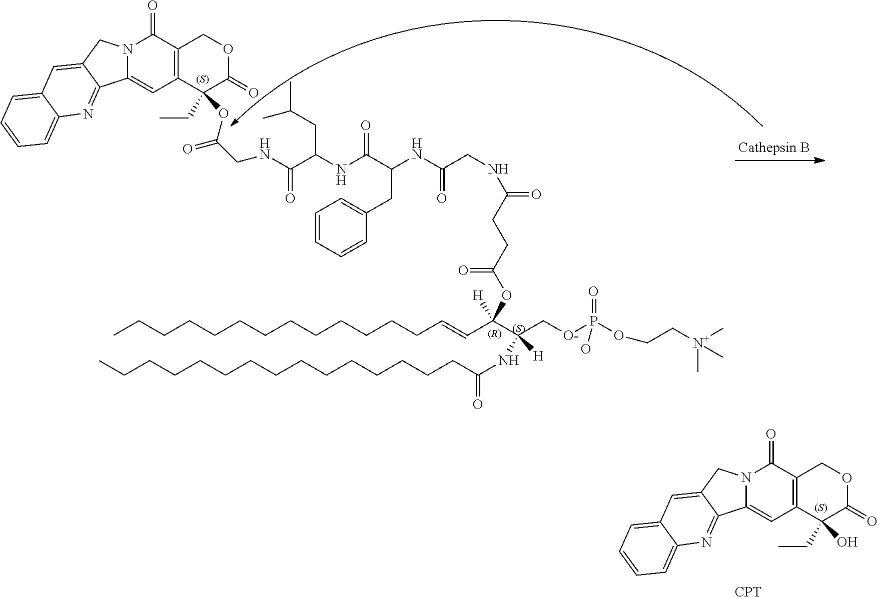

H. Synthetic Scheme for the Synthesis of SM-NN-CPT (Hydrazone bond)

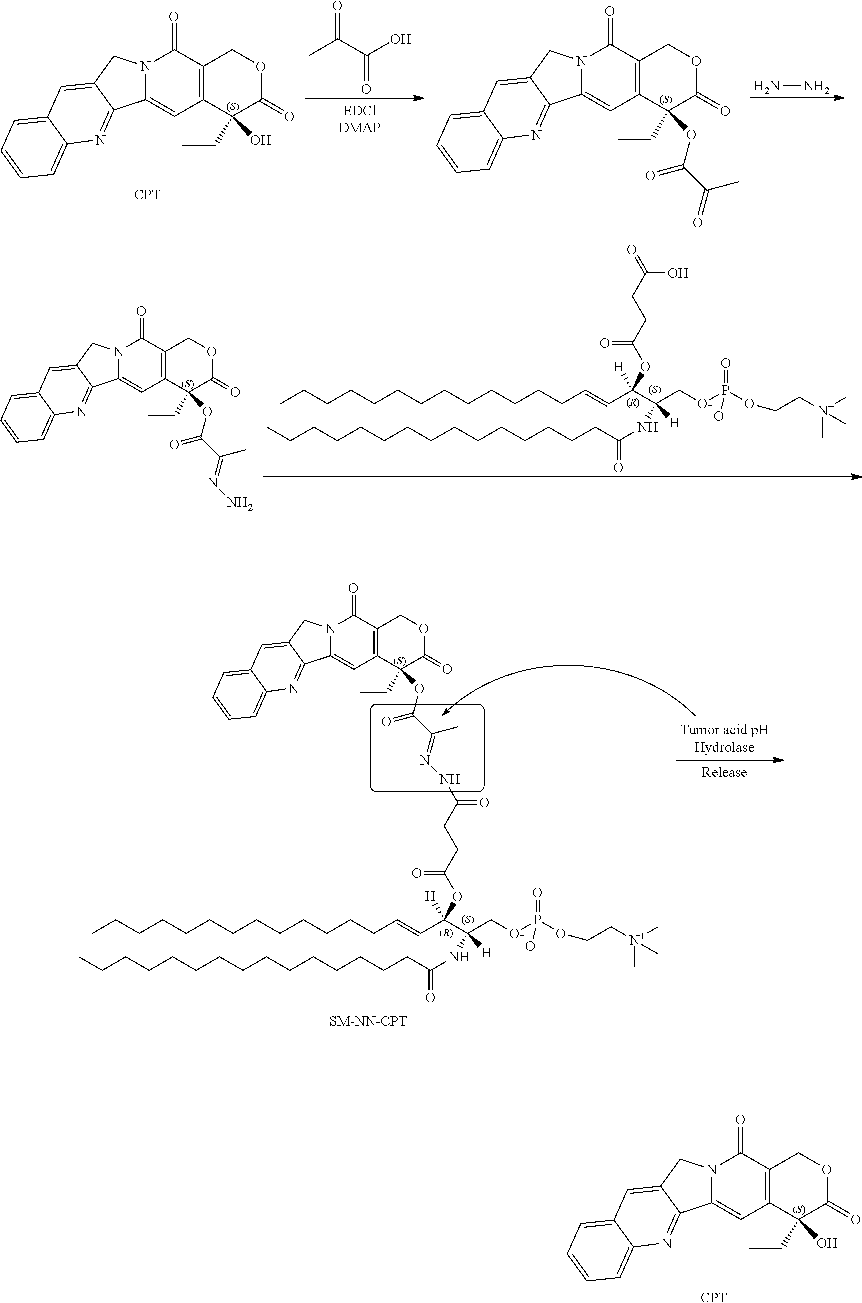

### I. Synthetic Scheme for the Synthesis of SM-PLGLAG-CPT (Pro-Leu-Gly-Leu-Ala-Gly)

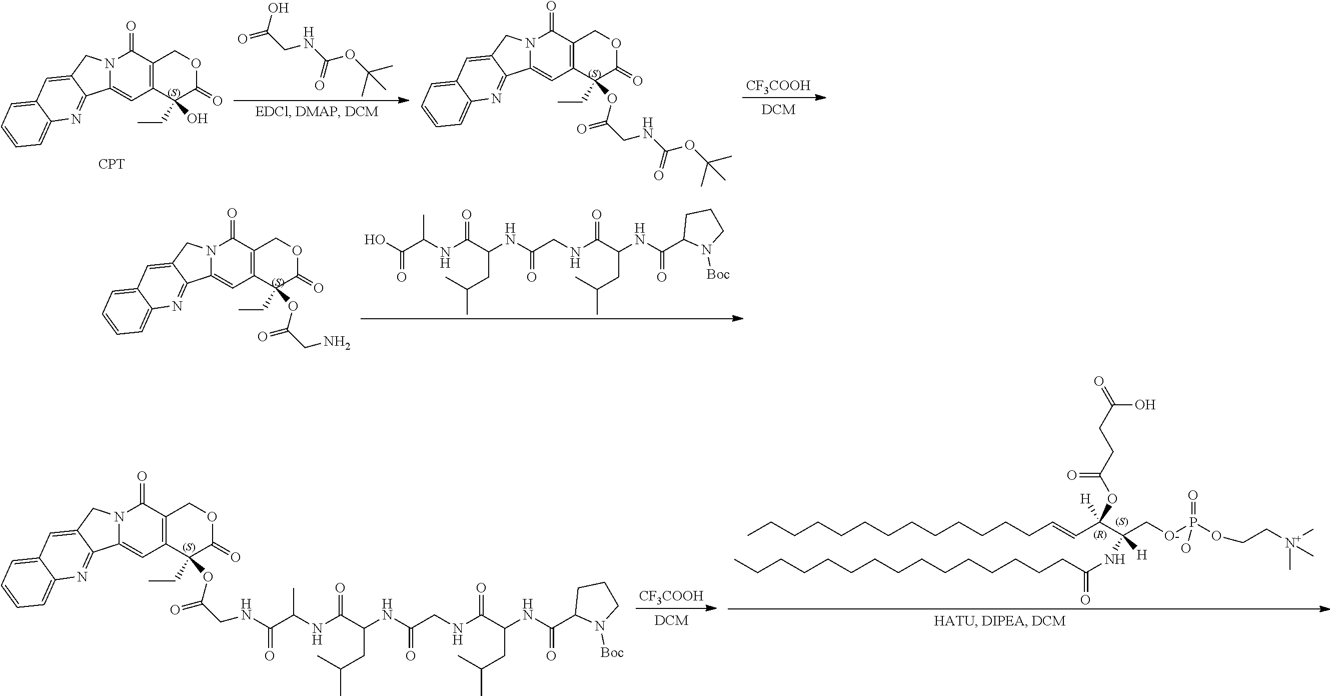

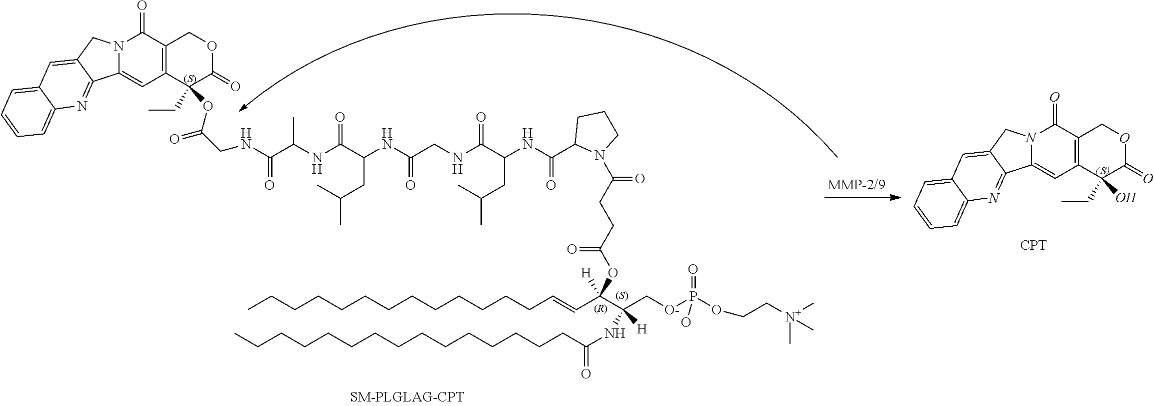

### J. Synthetic Scheme for the Synthesis of SM-AANK-CPT (Ala-Ala-Asn-Lys)

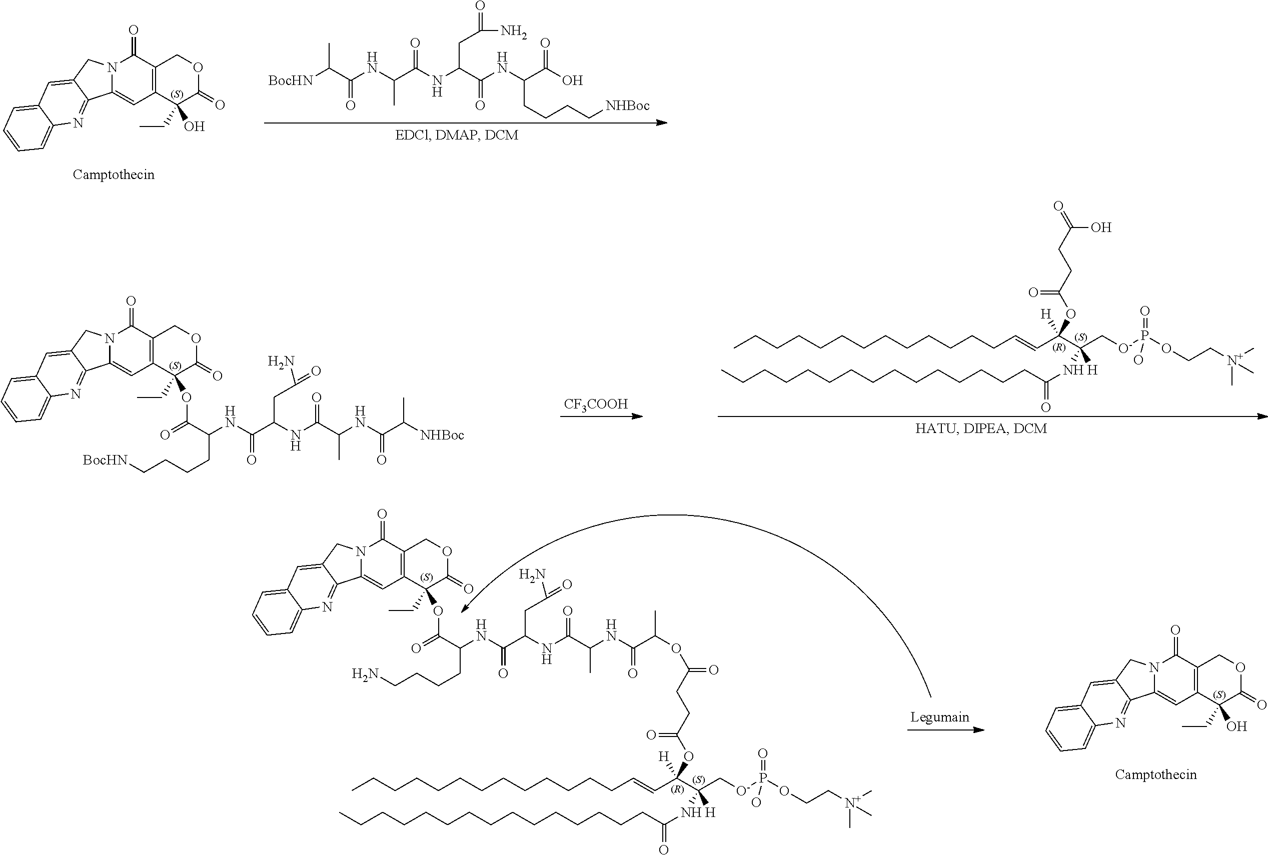

### SN-38-Based Sphingomyelin (SM) Drug Conjugates

Provided below is the proposed synthetic scheme for synthesizing SM drug conjugates featuring the cam ptothecin analogue, SN-38 as the drug.

### Synthetic Scheme for the Synthesis of SM-B-SN-38 (Borate Bond)

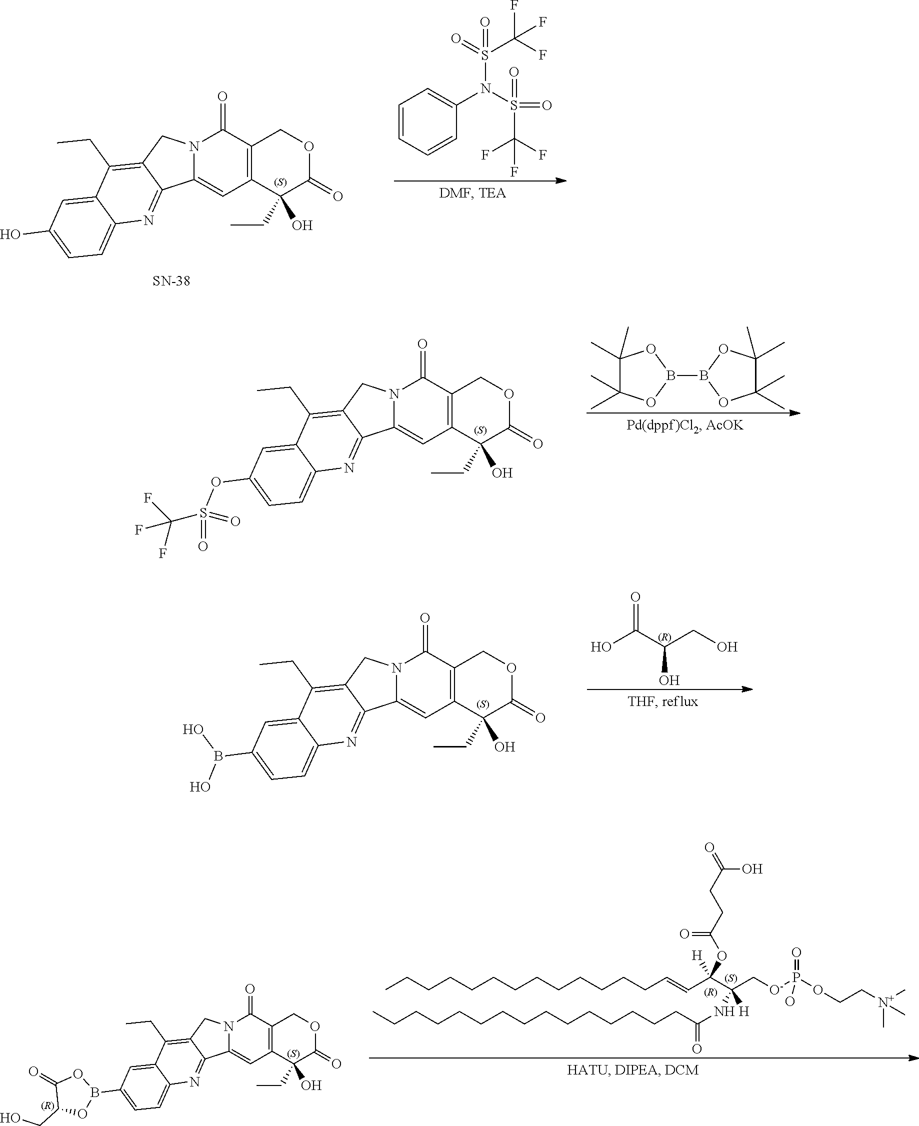

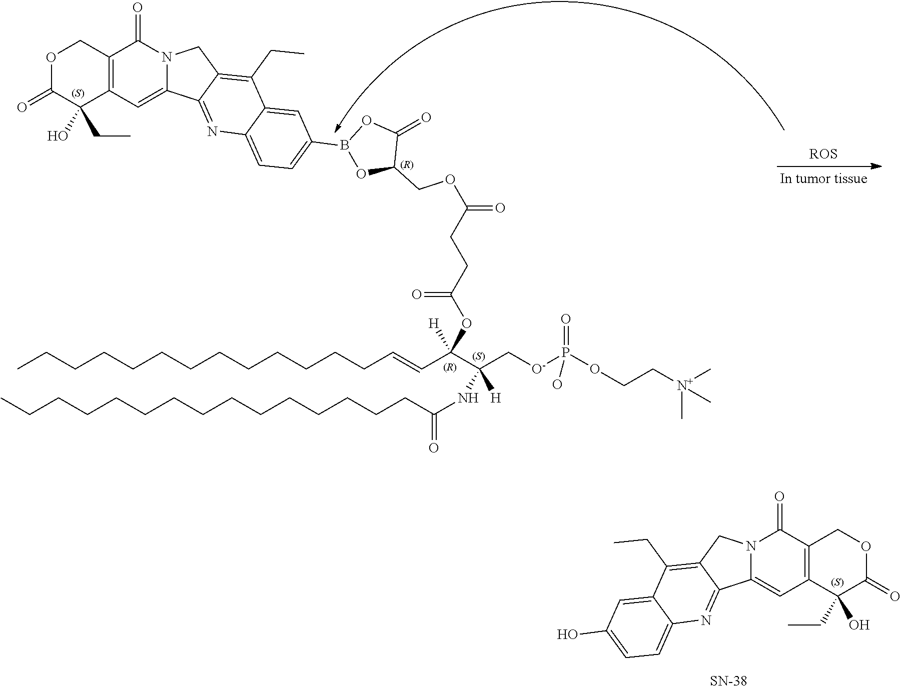

### PTX-Based Sphingomyelin (SM) Drug Conjugates

Provided below are the synthetic protocols and proposed synthetic schemes for synthesizing SM drug conjugates featuring paclitaxel (PTX) as the drug.

### A. Synthesis of SM-Ester-PTX

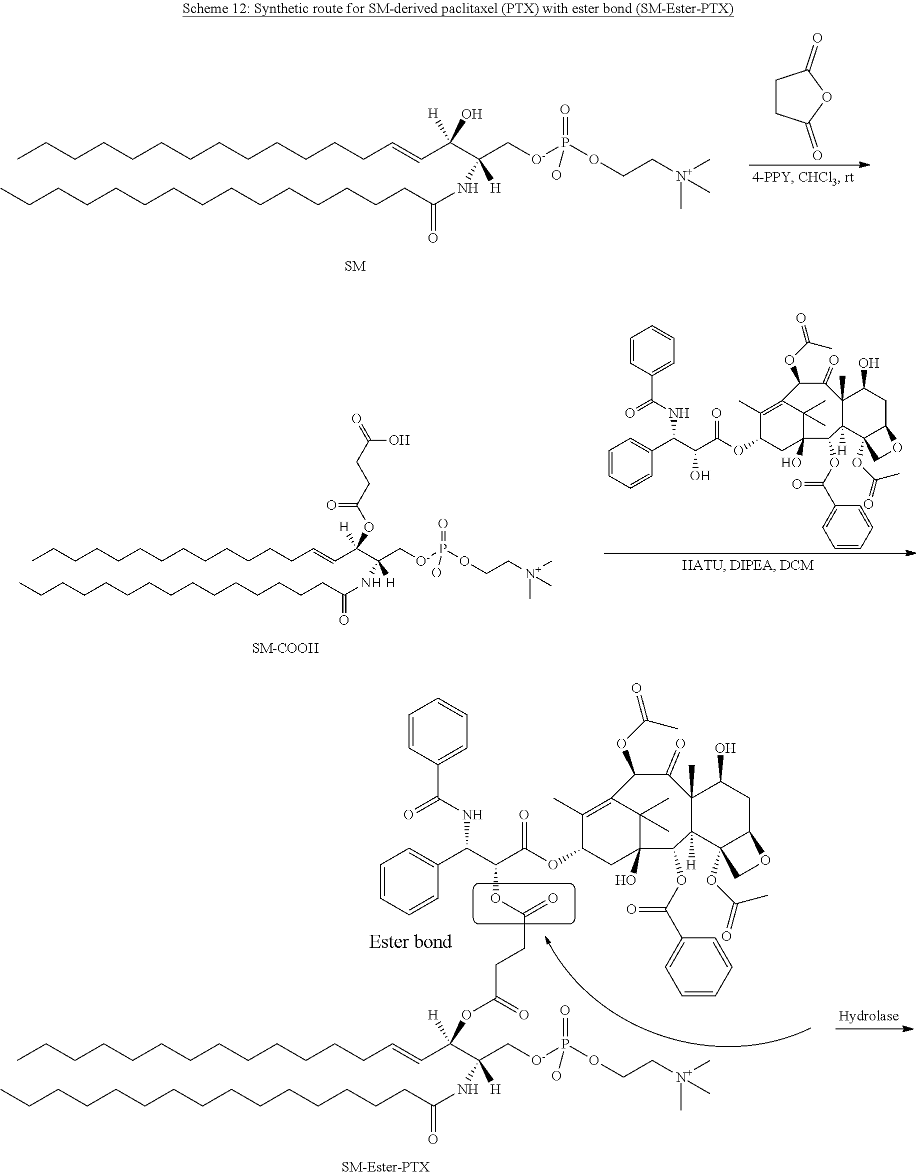

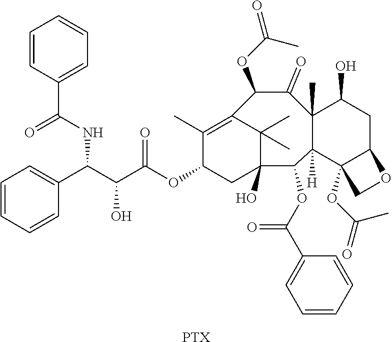

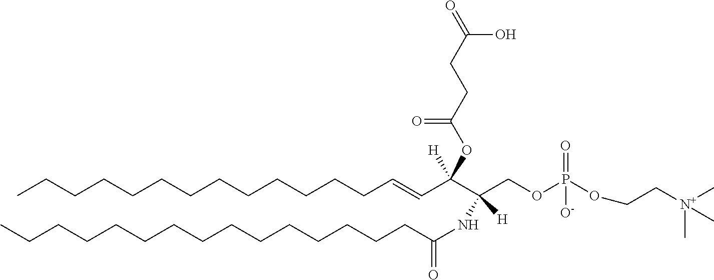 [SMILES: CCCCCCCCCCCCC/C=C/[C@@H](OC(=O)CCC(=O)O)[C@H](COP(=O)([O-])OCC[N+](C)(C)C)NC(=O)CCCCCCCCCCCCCCC]

### Sm-Coon

### (2S,3R,E)-3-((3-carboxypropanoyl)oxy)-2-palmitamidooctadec-4-en-1-yl (2-(trimethylammonio)ethyl) Phosphate

4-pyrrolidinopyridine (4-PPY, 44.4 mg, 0.3 mmol) was added to a solution of sphingomyelin (2.1 g, 3.0 mmol) and succinic anhydride (3 g, 30 mmol) in anhydrous CHCl₃ (100 mL). The solution was stirred at room temperature for 12 h and monitored by TLC. After completion of the reaction, the solvent was evaporated using rotary evaporator under vacuum, and the residue was purified by silica gel flash chromatography with CHCl₃/EtOH/H₂O (v/v/v, 300/200/36) as the elution solvent. White solid with 93% yield was garnered. R_{f}=0.23 (CHCl₃/EtOH/H₂O=300/200/36). ¹H NMR (400 MHz, CDCl₃) δ 6.98 (d, J=6.7 Hz, 1H), 5.75-5.68 (m, 1H), 5.39 (dd, J=15.0, 8.3 Hz, 1H), 5.28 (t, J=8.8 Hz, 1H), 4.29 (d, J=4.4 Hz, 3H), 3.94 (s, 2H), 3.77 (s, 2H), 3.28 (s, 9H), 2.64 (dd, J=13.1, 6.3 Hz, 2H), 2.39 (dd, J=14.3, 6.6 Hz, 2H), 2.12 (q, J=13.9 Hz, 2H), 1.97 (d, J=6.6 Hz, 2H), 1.55 (s, 2H), 1.28 (d, J=19.0 Hz, 46H), 0.88 (t, J=6.7 Hz, 6H). ¹³C NMR (101 MHz, CDCl₃) δ 174.74, 173.10, 172.06, 137.75, 125.20, 73.28, 65.76, 65.70, 64.26, 64.23, 59.20, 59.15, 54.32, 50.66, 50.61, 36.66, 32.25, 31.88, 29.74, 29.71, 29.70, 29.69, 29.63, 29.60, 29.56, 29.50, 29.46, 29.33, 28.92, 25.79, 22.64, 14.06. HRMS (ESI) m/z [M+H]⁺ for C₄₃H₈₄N₂O₉P calculated 803.5909, found 803.5928.

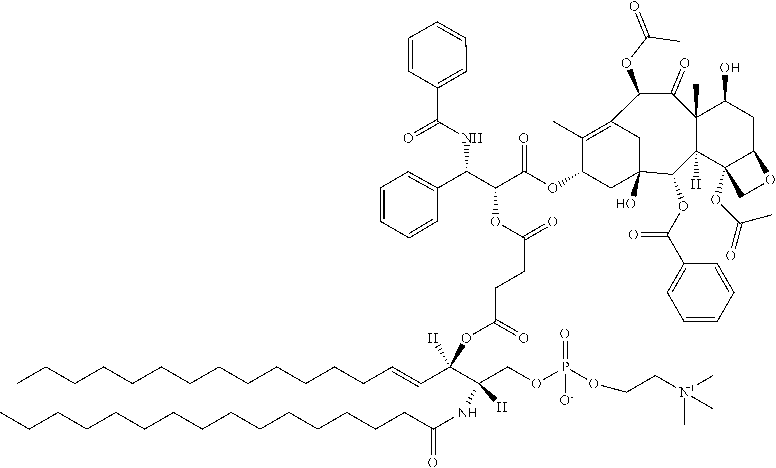 [SMILES: CCCCCCCCCCCCC/C=C/[C@@H](OC(=O)CCC(=O)O[C@@H](C(=O)O[C@H]1C[C@]2(O)CC(=C1C)[C@@H](OC(C)=O)C(=O)[C@@]1(C)[C@H]([C@@H]2OC(=O)c2ccccc2)[C@]2(OC(C)=O)CO[C@@H]2C[C@@H]1O)[C@@H](NC(=O)c1ccccc1)c1ccccc1)[C@H](COP(=O)([O-])OCC[N+](C)(C)C)NC(=O)CCCCCCCCCCCCCCC]

### SM-Ester-PTX

### (2S,3R,E)-3-((4-(((1 S,2R)-1-benzamido-3-(((2aR,4S,4aS,6R,9S,11 S,12S,12aR,12bS)-6,12b-diacetoxy-12-(benzoyloxy)-4,11-dihydroxy-4a, 8,13,13-tetramethyl-5-oxo-2a, 3,4,4a,5,6, 9,10,11,12,12 a,12 b-dodecahydro-1H-7,11-methanocyclodeca[3,4]benzo[1,2-b]oxet-9-yl) oxy)-3-oxo-1-phenylpropan-2-yl)oxy)-4-oxobutanoyl)oxy)-2-palmitamidooctadec-4-en-1-yl (2-(trimethylammonio)ethyl) Phosphate

DIPEA (2 mL) was added to a solution of SM-COOH (803.0 mg, 1.0 mmol) and HATU (380 mg, 1.0 mmol) in anhydrous DCM (50 mL). The reaction mixture was stirred at room temperature for 30 min. A solution of PTX (1.0 mmol) in 10 m L anhydrous DCM was added into the reaction and further stirred for 12 h. After completion of the reaction, the reaction mixture was washed with 50 mM HCl aqueous solution, and then with saturated brine. The organic layer was dried with anhydrous Na₂SO₄, the solvent was removed using rotary evaporator under vacuum, and the residue was purified by silica gel flash chromatography with CHCl₃/EtOH/H₂O (v/v/v, 300/200/36) as the eluting solvent. White solid with 88% yield was achieved. R_{f}=0.38 (CHCl₃/EtOH/H₂O=300/200/36). ¹H NMR (400 MHz, CDCl₃) δ 9.76 (s, 1H), 8.05 (t, J=7.9 Hz, 4H), 7.69 (t, J=7.4 Hz, 2H), 7.57 (t, J=6.8 Hz, 4H), 7.41 (d, J=6.7 Hz, 3H), 7.33 (t, J=7.6 Hz, 2H), 7.05-7.00 (m, 1H), 6.68 (d, J=8.7 Hz, 1H), 6.29 (s, 1H), 5.94 (t, J=8.7 Hz, 1H), 5.67 (tt, J=16.2, 8.1 Hz, 4H), 5.57 (d, J=6.9 Hz, 1H), 5.32 (dd, J=15.2, 8.1 Hz, 1H), 5.03 (d, J=8.8 Hz, 1H), 4.88 (d, J=9.4 Hz, 1H), 4.29 (s, 3H), 4.23 (d, J=8.4 Hz, 1H), 4.09 (d, J=8.3 Hz, 1H), 4.02 (d, J=8.6 Hz, 1H), 3.73-3.64 (m, 2H), 3.63-3.55 (m, 3H), 3.30 (d, J=8.1 Hz, 1H), 3.22 (s, 9H), 2.97-2.83 (m, 2H), 2.68 (d, J=5.0 Hz, 1H), 2.64 (s, 1H), 2.55 (s, 2H), 2.44 (d, J=9.1 Hz, 5H), 2.18 (s, 3H), 2.12-2.07 (m, 2H), 1.97-1.90 (m, 3H), 1.80 (s, 3H), 1.67 (s, 1H), 1.63 (s, 3H), 1.54 (s, 2H), 1.26 (s, 46H), 1.16 (s, 3H), 1.09 (s, 3H), 0.88 (t, J=6.7 Hz, 6H). ¹³C NMR (101 MHz, CDCl₃) δ 203.50, 173.01, 171.92, 171.31, 170.41, 169.56, 167.57, 166.79, 141.44, 137.86, 137.72, 134.45, 133.89, 132.69, 131.50, 130.12, 129.22, 128.67, 128.61, 128.50, 128.36, 128.25, 127.79, 124.91, 84.08, 80.89, 78.62, 77.27, 76.20, 75.76, 75.69, 74.65, 73.86, 71.32, 70.69, 66.82, 66.76, 63.75, 58.67, 58.63, 57.89, 55.05, 54.37, 50.81, 50.74, 46.54, 43.03, 36.71, 34.46, 32.29, 31.93, 30.11, 29.74, 29.68, 29.64, 29.61, 29.53, 29.48, 29.37, 29.20, 28.92, 26.52, 25.94, 23.30, 22.69, 21.57, 21.03, 14.77, 14.13, 10.02. HRMS (ESI) m/z [M+H]⁺ for C₉₀H₁₃₃N₃O₂₂P calculated 1638.9113, found 1638.9172.

### B. Synthesis of SM-SS-PTX

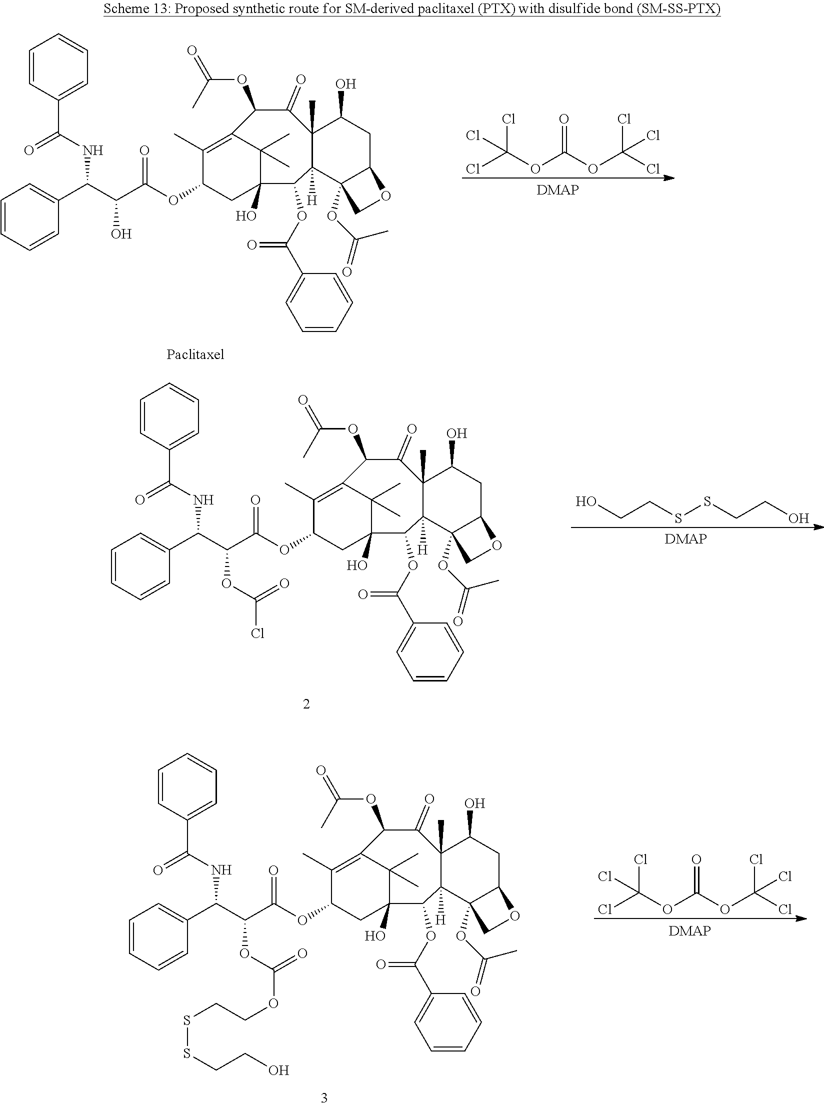

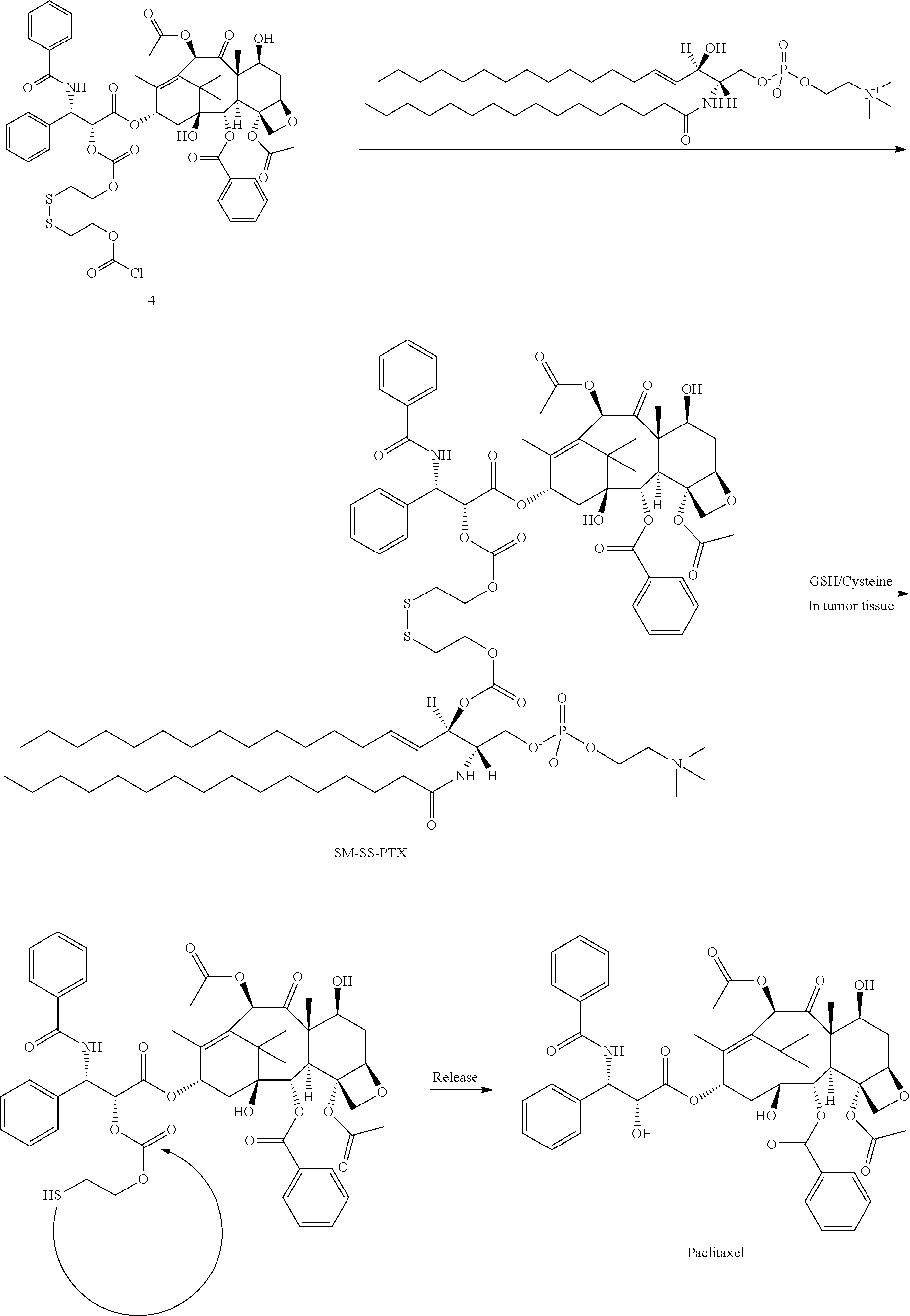

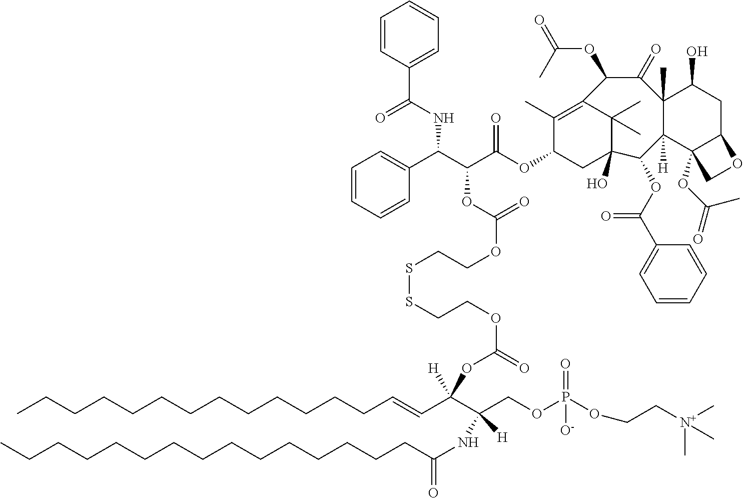 [SMILES: CCCCCCCCCCCCC/C=C/[C@@H](OC(=O)OCCSSCCOC(=O)O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1)[C@H](COP(=O)([O-])OCC[N+](C)(C)C)NC(=O)CCCCCCCCCCCCCCC]

### Sm-Ss-Ptx

### (3S,4R,17R,18S)-4-((((2aR,4S,4aS,6R,9S, 11S,12S,12aR,12bS)-6,12b-diacetoxy-12-(benzoyloxy)-4,11-dihydroxy-4a,8,13,13-tetramethyl-5-oxo-2a, 3, 4, 4a, 5,6, 9,10,11,12,12 a,12b-dodecahydro-1H-7,11-methanocyclodeca[3,4]benzo[1,2-b]oxet-9-yl) oxy)carbonyl)-1,6,15-tri oxo-18-palmitamido-17-((E)-pentadec-1-en-1-yl)-1,3-diphenyl-5,7,14,16-tetraoxa-10,11-dithia-2-azanonadecan-19-yl (2-(trimethylammonio)ethyl) Phosphate

### C. Synthesis of SM-CSS-PTX

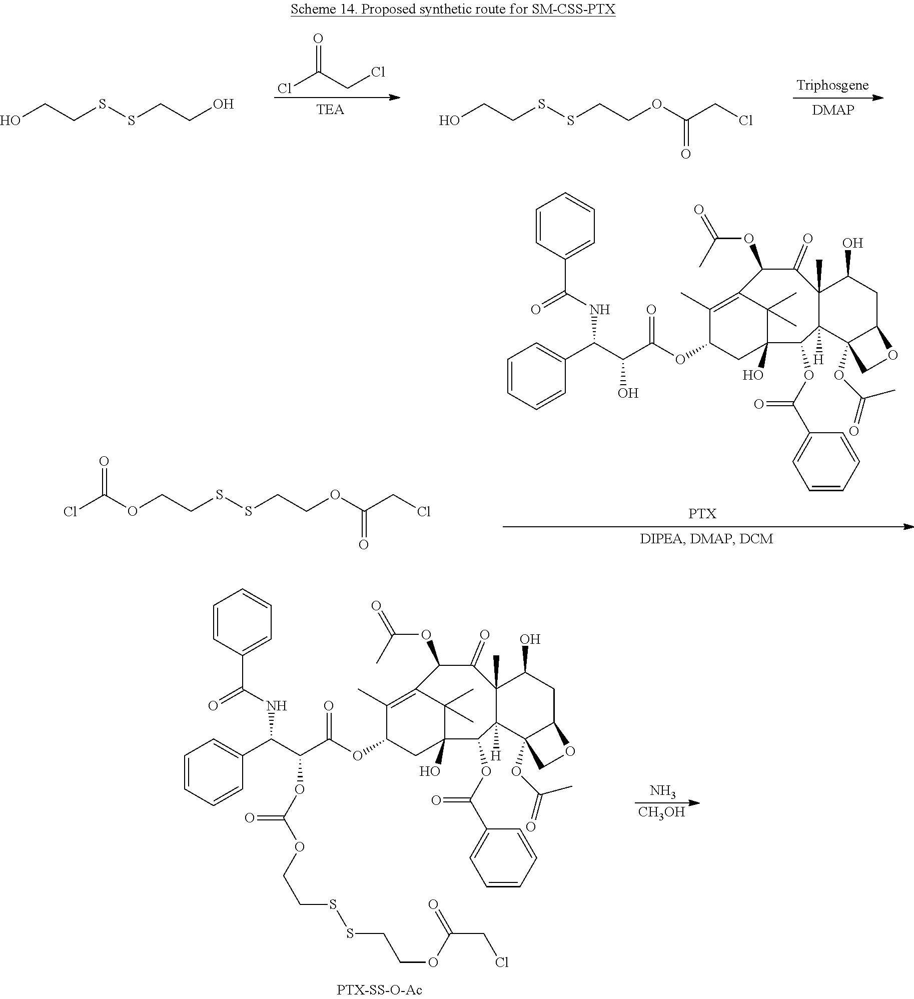

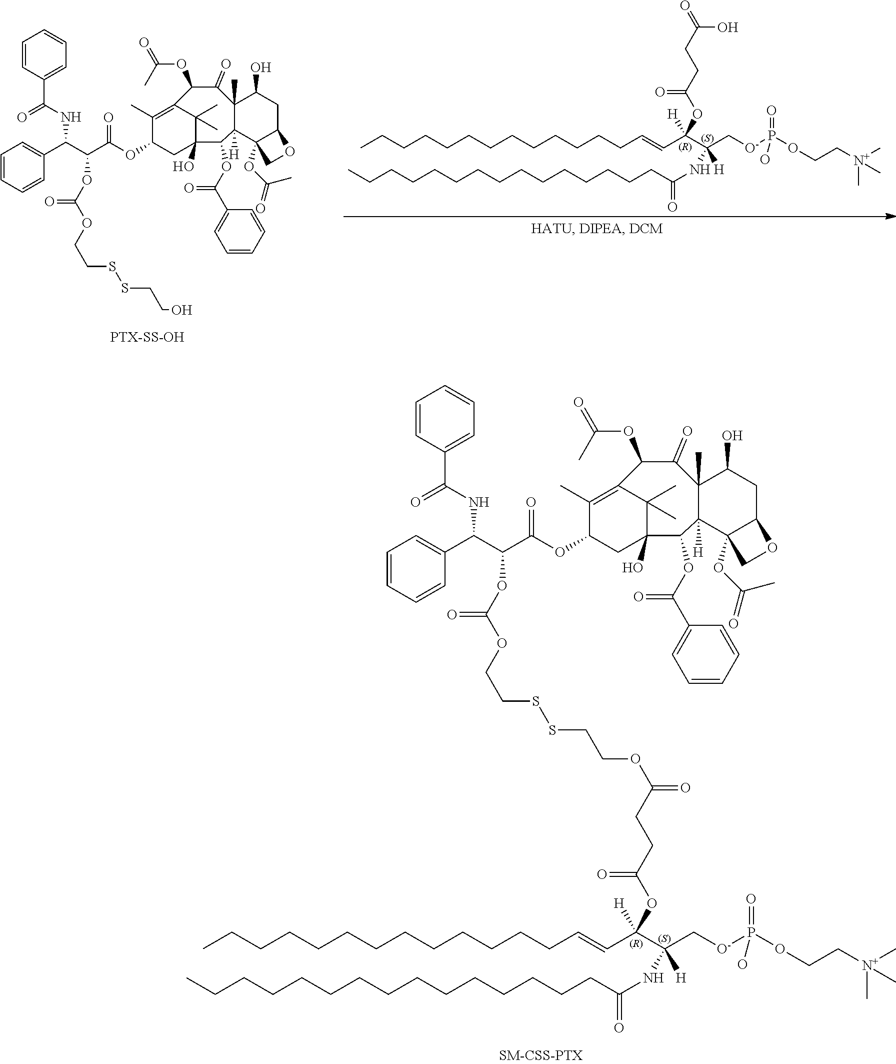

### D. Synthesis of SM-Glycine-PTX

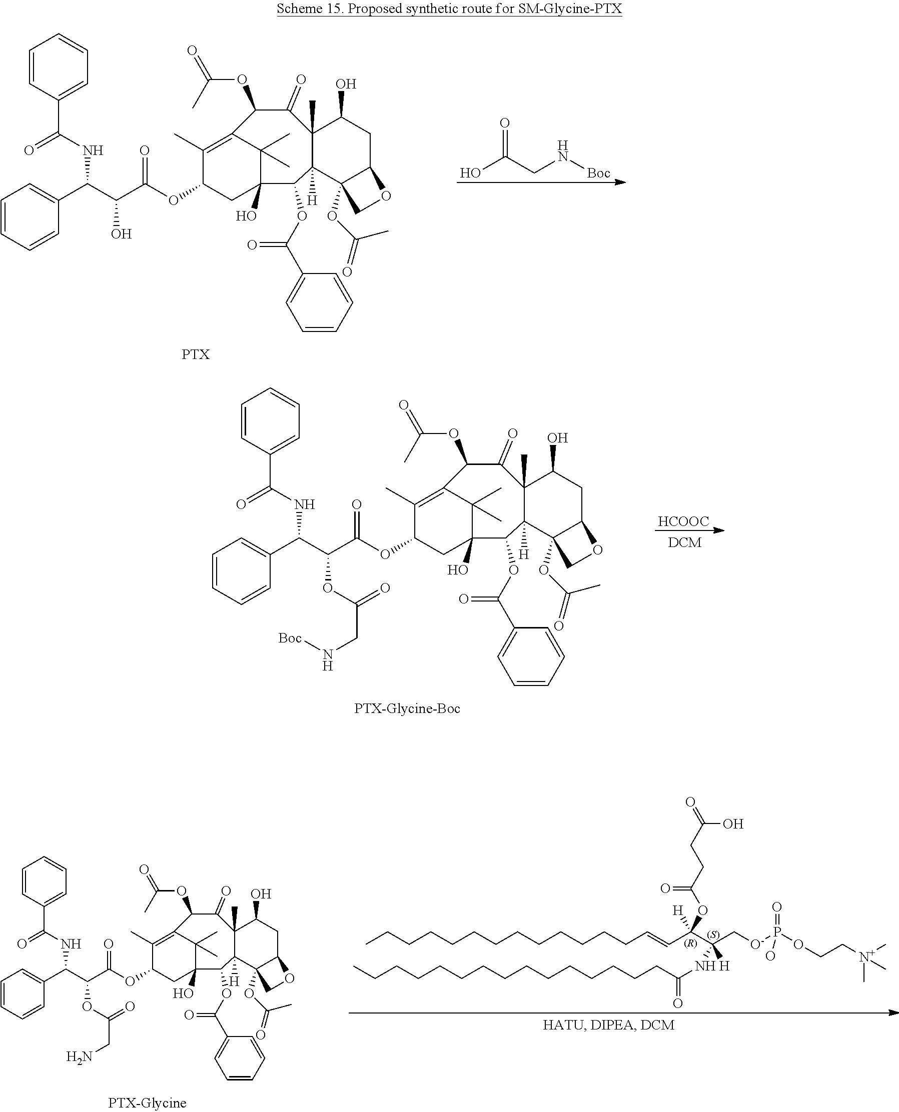

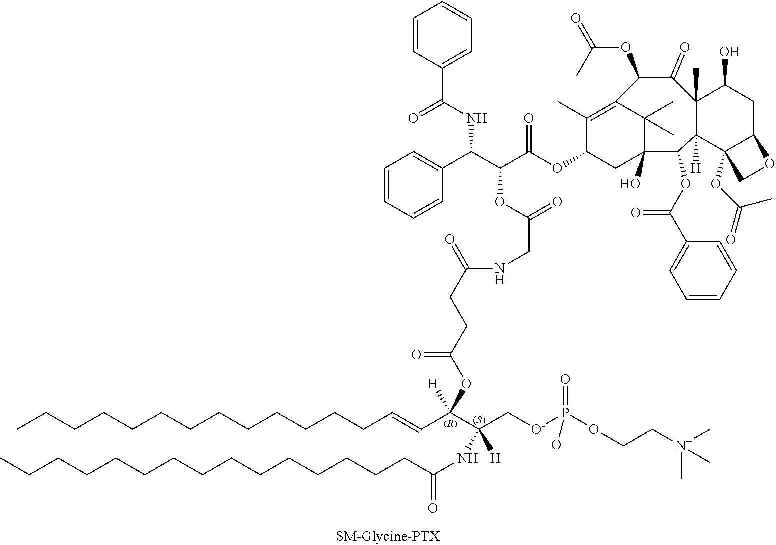

### DTX-Based Sphingomyelin (SM) Drug Conjugates

Provided below are the synthetic protocols and proposed synthetic schemes for synthesizing SM drug conjugates featuring docetaxel (DTX) as the drug.

### A. Synthesis of SM-Ester-DTX

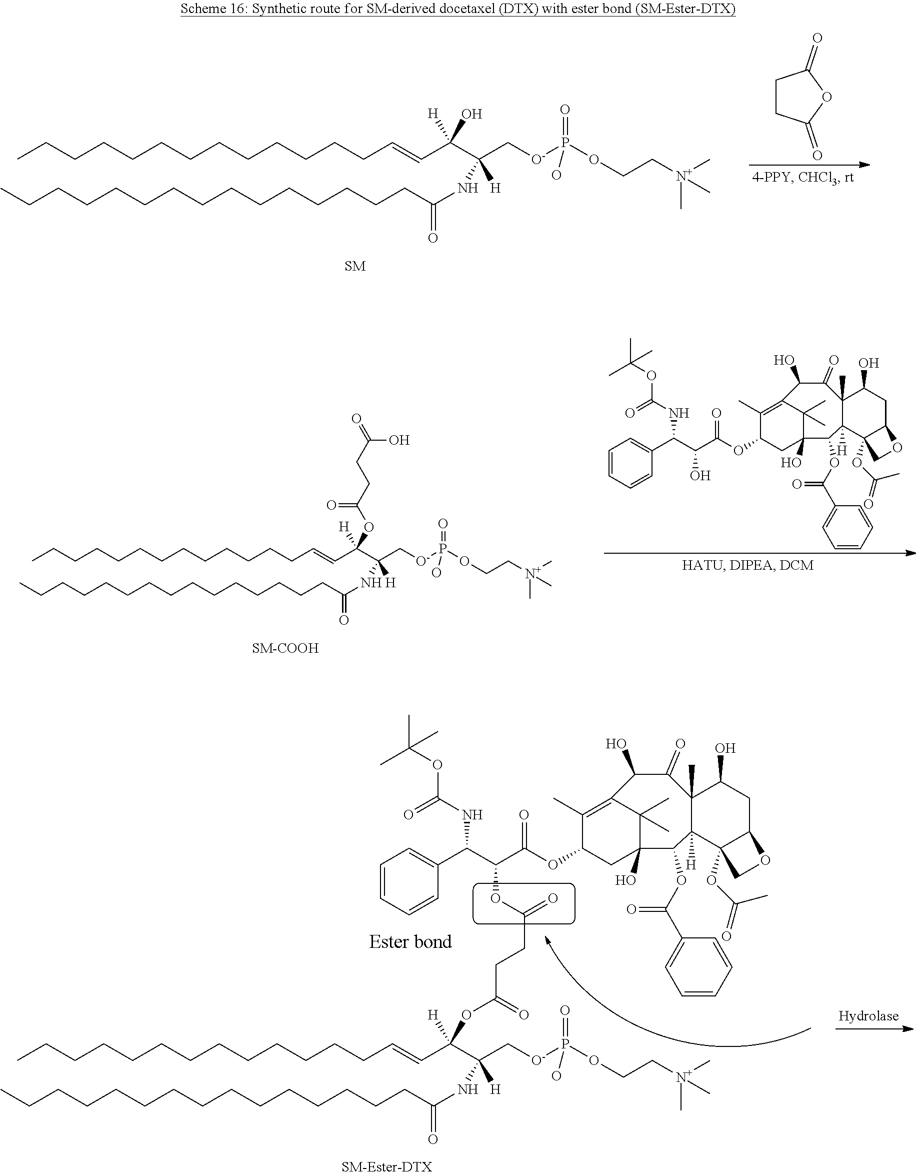

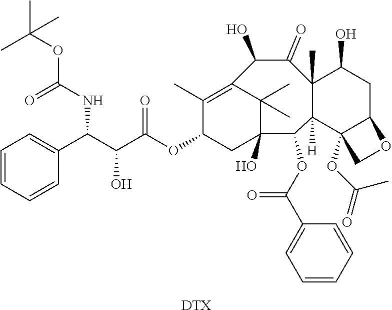

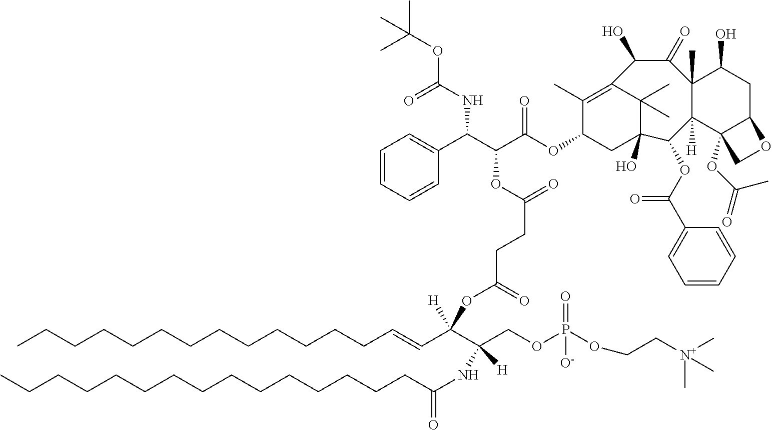 [SMILES: CCCCCCCCCCCCC/C=C/[C@@H](OC(=O)CCC(=O)O[C@@H](C(=O)O[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](O)C(=C1C)C2(C)C)[C@@H](NC(=O)OC(C)(C)C)c1ccccc1)[C@H](COP(=O)([O-])OCC[N+](C)(C)C)NC(=O)CCCCCCCCCCCCCCC]

### SM-Ester-DTX

### (6S,7R,14R,15S)-7-((((2aR,4S,4aS,6R,9S, 11S,12S,12aR,12bS)-12b-acetoxy-12-(benzoyloxy)-4,6,11-trihydroxy-4a,8,13,13-tetramethyl-5-oxo-2a, 3,4,4a,5,6,9,10, 11, 12, 12 a,12b-dodecahydro-1H-7,11-methanocyclodeca[3,4]benzo[1,2-b]oxet-9-yl) oxy)carbonyl)-2,2-dimethyl-4, 9,12-trioxo-15-palmitamido-14-((E)-pentadec-1-en-1-yl)-6-phenyl-3,8,13-trioxa-5-azahexadecan-16-yl (2-(trimethylammonio)ethyl) Phosphate

DIPEA (2 mL) was added to a solution of SM-COOH (803.0 mg, 1.0 mmol) and HATU (380 mg, 1.0 mmol) in anhydrous DCM (50 mL). The reaction mixture was stirred at room temperature for 30 min. A solution of DTX (1.0 mmol) in 10 mL anhydrous DCM was added into the reaction and further stirred for 12 h. After completion of the reaction, the reaction mixture was washed with 50 mM HCl aqueous solution, and then with saturated brine. The organic layer was dried with anhydrous Na₂SO₄, the solvent was removed using rotary evaporator under vacuum, and the residue was purified by silica gel flash chromatography with CHCl₃/EtOH/H₂O (v/v/v, 300/200/36) as the eluting solvent. White solid with 76% yield was achieved. R_{f}=0.41 (CHCl₃/EtOH/H₂O=300/200/36). ¹H NMR (400 MHz, DMSO-d₆) δ 8.12 (d, J=8.9 Hz, 1H), 7.95 (d, J=7.5 Hz, 2H), 7.76-7.72 (m, 1H), 7.66 (t, J=7.4 Hz, 2H), 7.43-7.39 (m, 2H), 7.36 (d, J=7.0 Hz, 1H), 7.17-7.13 (m, 1H), 5.78 (d, J=5.9 Hz, 1H), 5.70 (dd, J=17.3, 7.2 Hz, 2H), 5.63 (s, 1H), 5.34 (d, J=7.5 Hz, 1H), 5.30 (d, J=4.7 Hz, 1H), 5.17-5.09 (m, 2H), 5.00-4.94 (m, 1H), 4.87 (d, J=9.5 Hz, 1H), 4.34 (s, 1H), 4.02 (dd, J=17.4, 6.3 Hz, 6H), 3.95 (d, J=7.8 Hz, 1H), 3.71 (s, 2H), 3.64 (d, J=5.3 Hz, 1H), 3.58 (d, J=6.7 Hz, 1H), 3.51 (s, 2H), 3.11 (s, 9H), 2.74-2.54 (m, 5H), 2.25-2.10 (m, 5H), 2.04 (dd, J=14.3, 7.1 Hz, 2H), 1.93 (d, J=6.3 Hz, 2H), 1.73-1.55 (m, 6H), 1.46 (s, 6H), 1.39 (s, 9H), 1.23 (s, 46H), 0.95 (s, 6H), 0.85 (t, J=6.6 Hz, 6H). ¹³C NMR (101 MHz, DMSO-d₆) δ210.01, 172.40, 171.78, 170.95, 169.82, 169.66, 165.66, 155.65, 137.91, 137.63, 136.10, 136.03, 133.79, 130.55, 129.93, 129.02, 128.88, 128.45, 128.04, 125.80, 84.35, 80.68, 78.67, 77.22, 75.79, 75.75, 75.29, 73.93, 73.84, 71.30, 70.58, 65.83, 65.79, 63.73, 58.97, 58.92, 57.36, 55.79, 53.55, 51.44, 51.39, 46.58, 43.18, 37.05, 36.02, 34.93, 32.19, 31.75, 29.56, 29.54, 29.51, 29.48, 29.47, 29.18, 29.17, 29.14, 28.82, 28.55, 26.82, 25.81, 23.00, 22.53, 21.22, 14.33, 14.17, 10.24. HRMS (ESI) m/z [M+H]⁺ for C₈₆H₁₃₅N₃O₂₂P calculated 1592.9269, found 1592.9338.

### B. Synthetic Scheme for the Synthesis of SM-SS-DTX

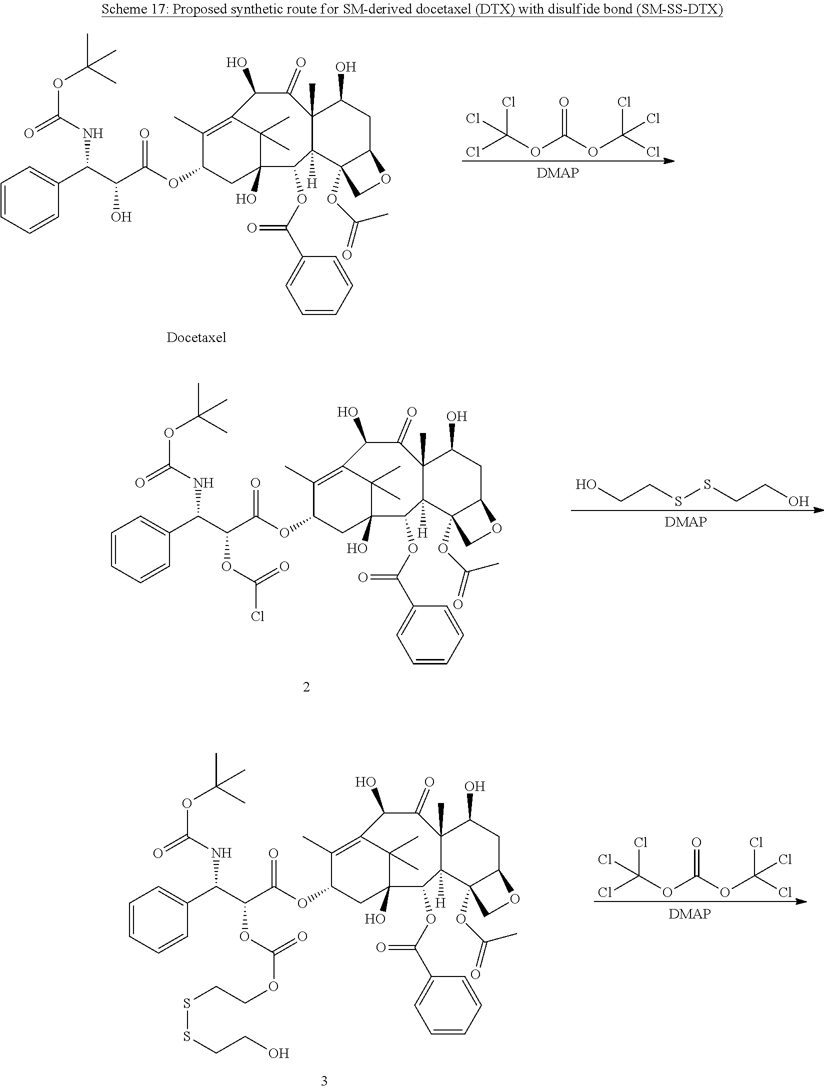

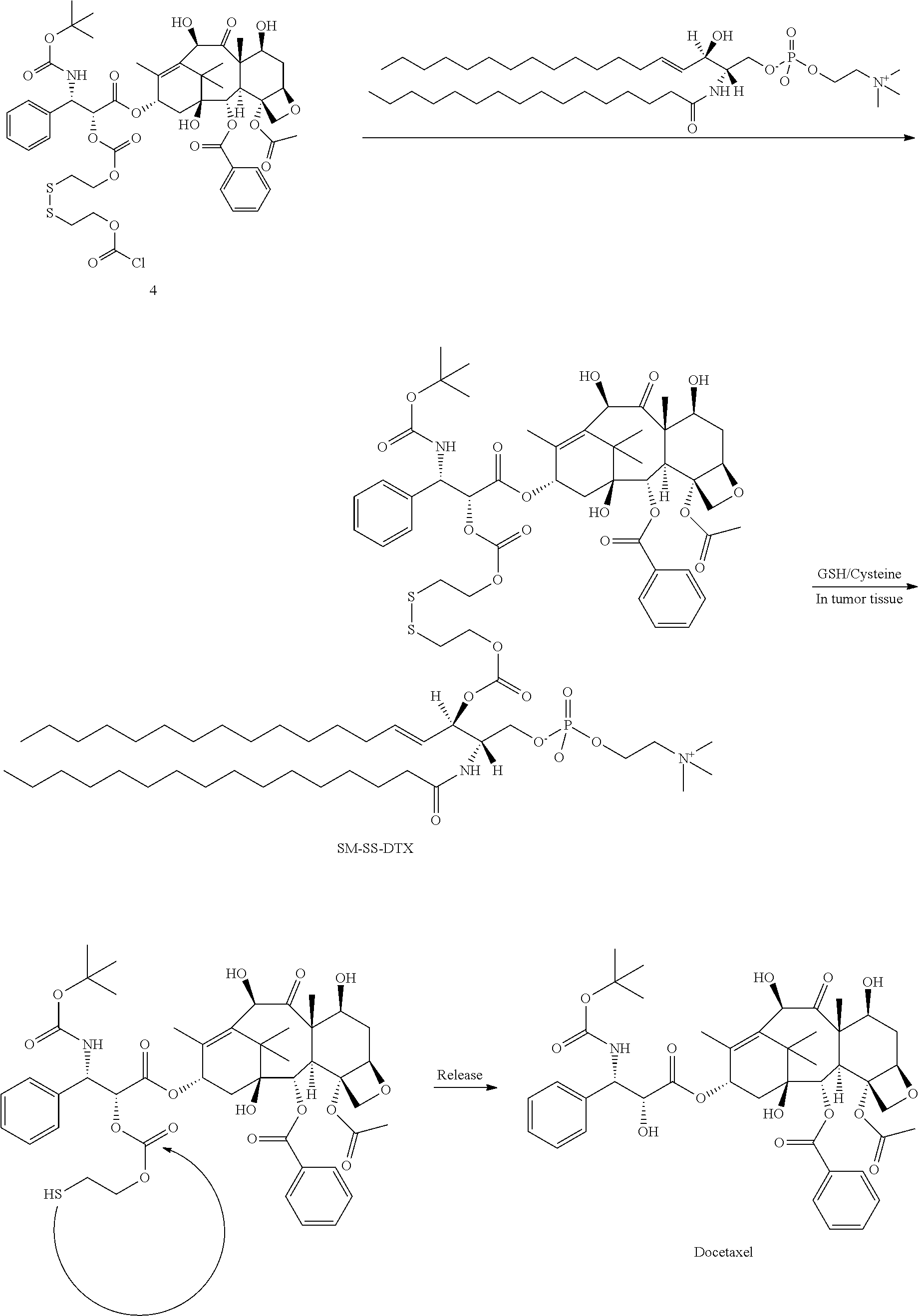

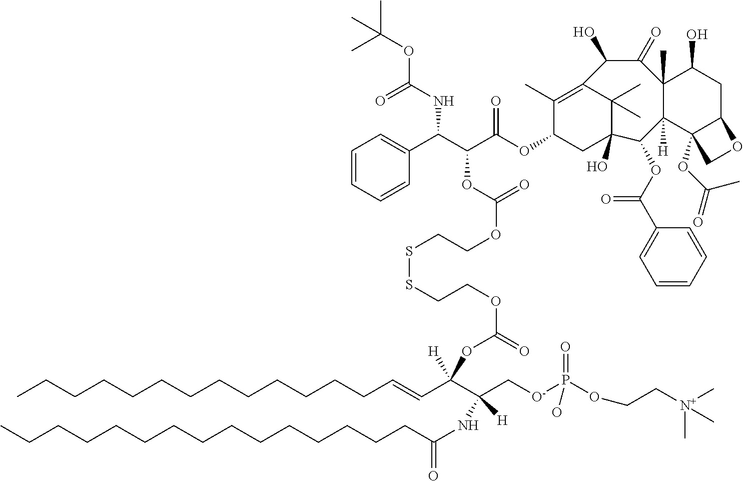

### Sm-Ss-Dtx

### (6S,7R,20R,21 S)-7-((((2aR,4S,4aS,6R,9S,11 S,12S,12aR,12bS)-12b-acetoxy-12-(benzoyloxy)-4,6,11-trihydroxy-4a,8,13,13-tetramethyl-5-oxo-2a, 3,4,4a,5,6,9,10,11,12, 12 a,12b-dodecahydro-1H-7,11-methanocyclodeca[3,4]benzo[1,2-b]oxet-9-yl) oxy)carbonyl)-2,2-dimethyl-4, 9,18-trioxo-21-palmitamido-20-((E)-pentadec-1-en-1-yl)-6-phenyl-3,8,10,17,19-pentaoxa-13,14-dithia-5-azadocosan-22-yl (2-(trimethylammonio)ethyl) Phosphate

### C. Synthetic Scheme for the Synthesis of SM-CSS-DTX

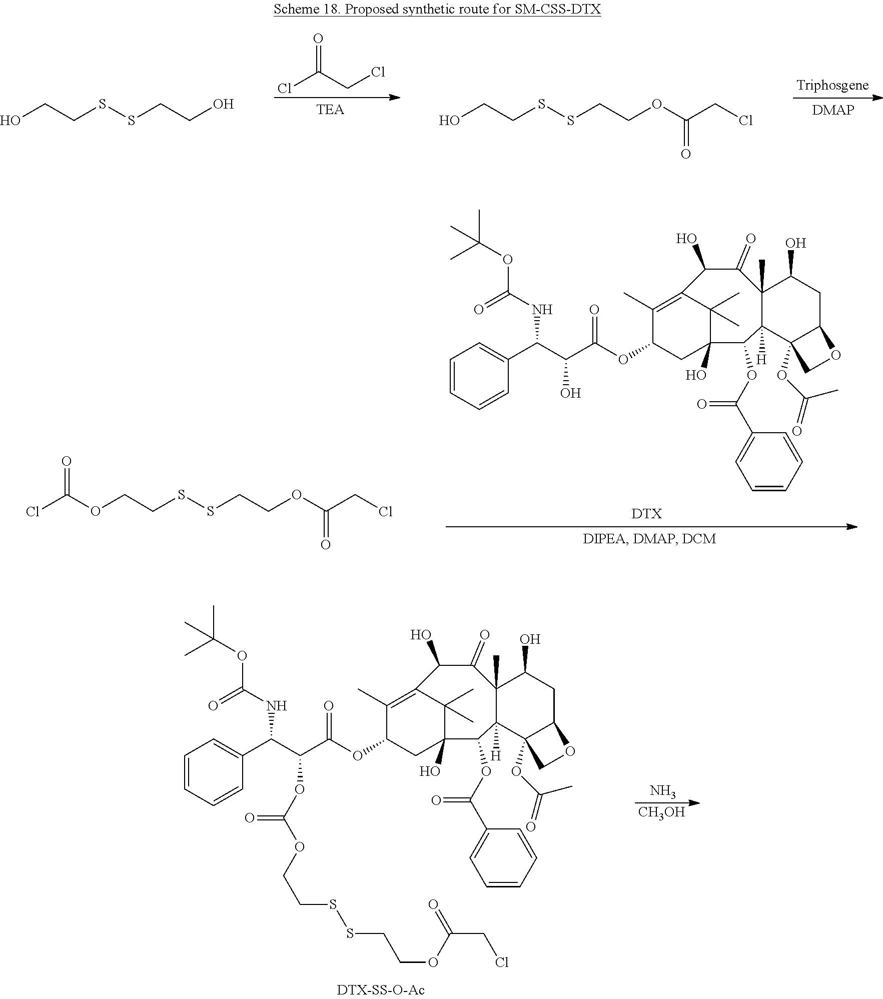

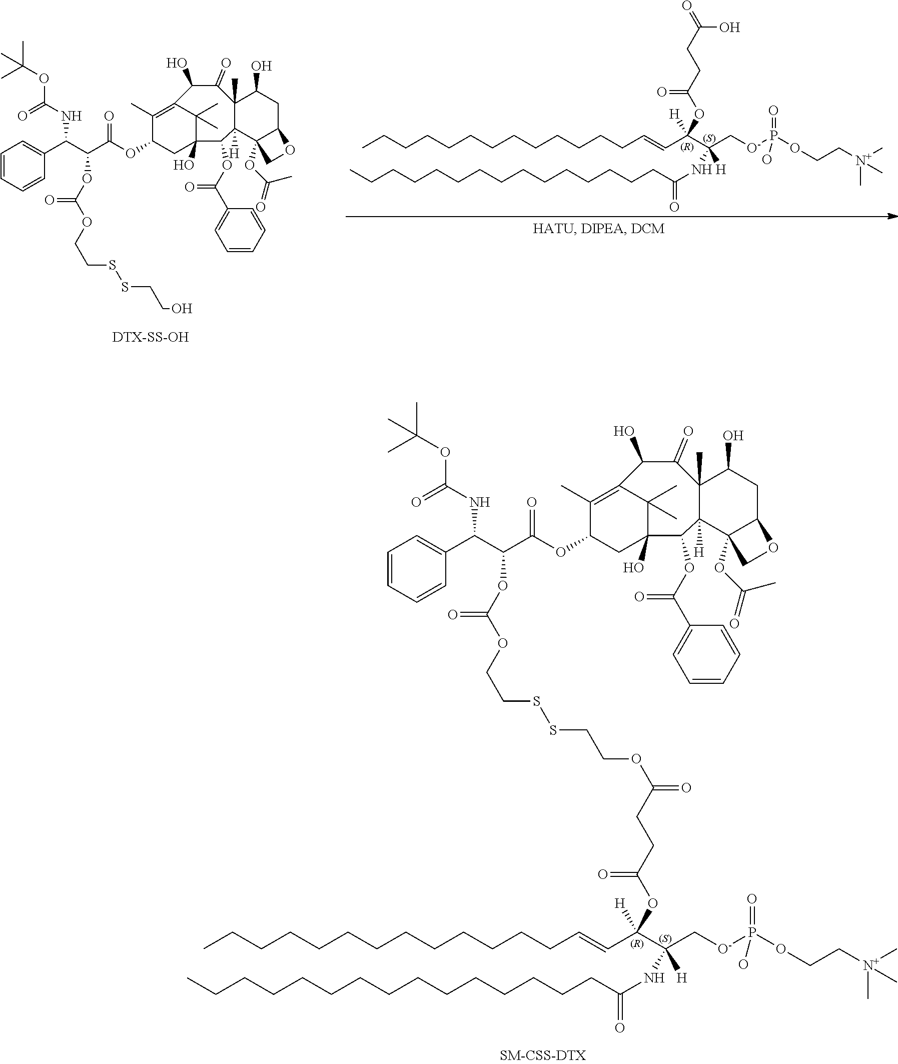

### D. Synthetic Scheme for the Synthesis of SM-Glycine-DTX

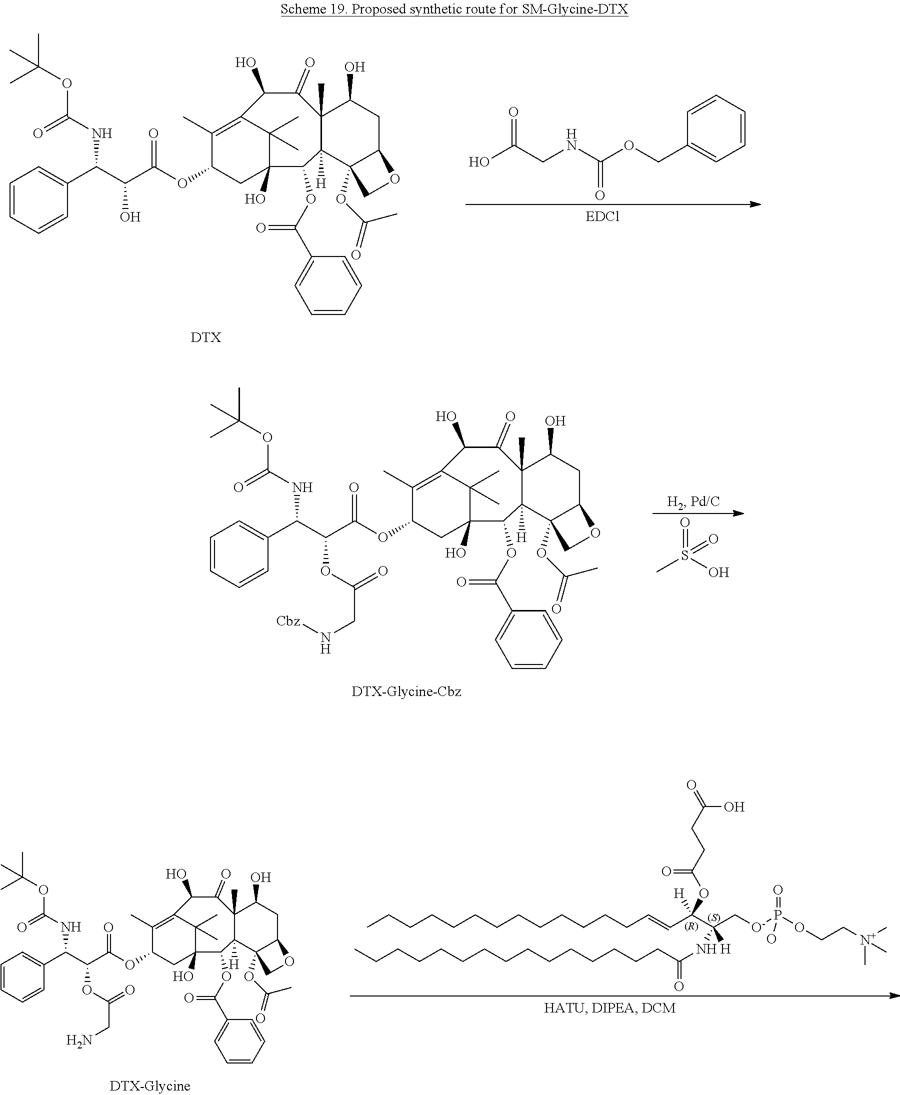

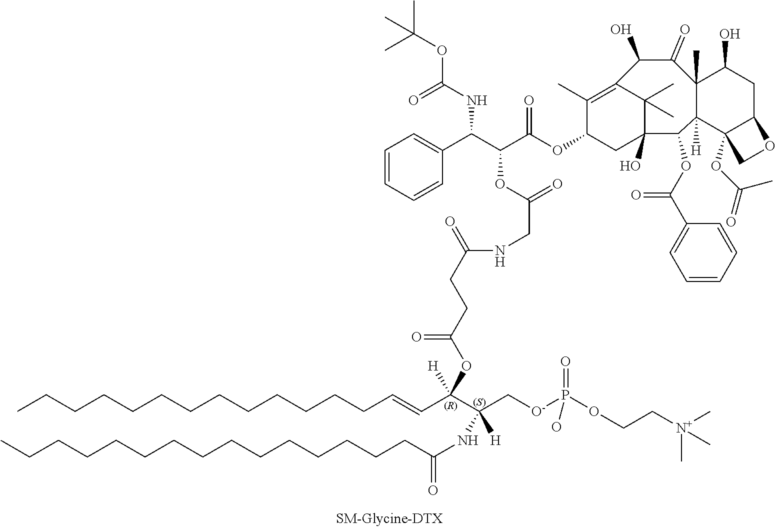

### EPA-Based Sphingomyelin (SM) Drug Conjugates

Provided below is the synthetic protocol for synthesizing SM drug conjugates featuring Epacadostat (EPA) as the drug.

### Synthesis of SM-Ester-EPA

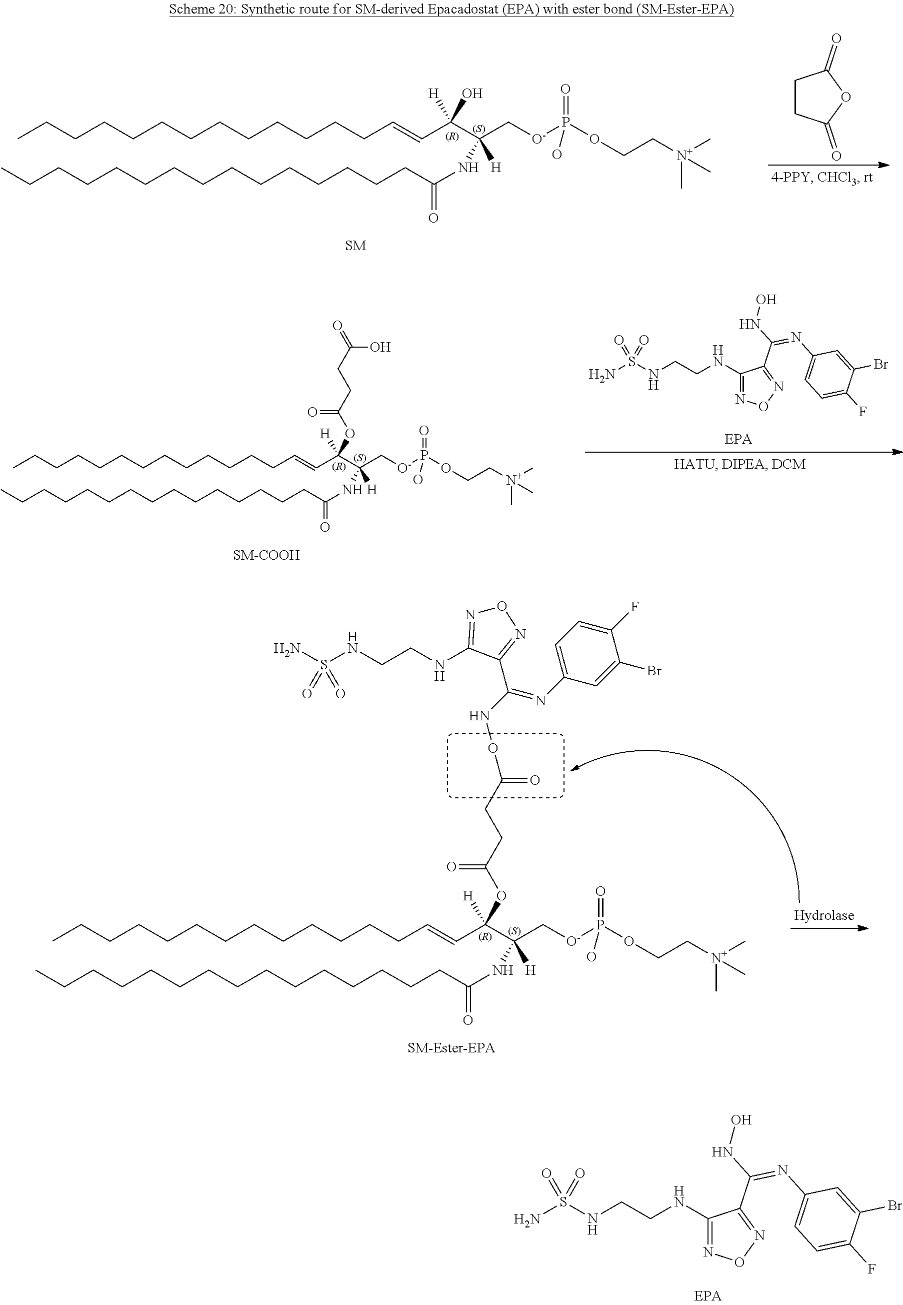

### (2S,3R,E)-3-((3-carboxypropanoyl)oxy)-2-palmitamidooctadec-4-en-1-yl (2-(trimethylammonio)ethyl) Phosphate (SM-COOH)

4-pyrrolidinopyridine (4-PPY, 44.4 mg, 0.3 mmol) was added to a solution of sphingomyelin (2.1 g, 3.0 mmol) and succinic anhydride (3 g, 30 mmol) in anhydrous CHCl₃ (100 mL). The solution was stirred at room temperature for 12 h and monitored by TLC. After completion of the reaction, the solvent was evaporated using rotary evaporator under vacuum, and the residue was purified by silica gel flash chromatography with CHCl₃/EtOH/H₂O (v/v/v, 300/200/36) as the elution solvent. White solid with 93% yield was garnered. R_{f}=0.23 (CHCl₃/EtOH/H₂O=300/200/36). ¹H NMR (400 MHz, CDCl₃) δ 6.98 (d, J=6.7 Hz, 1H), 5.75-5.68 (m, 1H), 5.39 (dd, J=15.0, 8.3 Hz, 1H), 5.28 (t, J=8.8 Hz, 1H), 4.29 (d, J=4.4 Hz, 3H), 3.94 (s, 2H), 3.77 (s, 2H), 3.28 (s, 9H), 2.64 (dd, J=13.1, 6.3 Hz, 2H), 2.39 (dd, J=14.3, 6.6 Hz, 2H), 2.12 (q, J=13.9 Hz, 2H), 1.97 (d, J=6.6 Hz, 2H), 1.55 (s, 2H), 1.28 (d, J=19.0 Hz, 46H), 0.88 (t, J=6.7 Hz, 6H). ¹³C NMR (101 MHz, CDCl₃) δ 174.74, 173.10, 172.06, 137.75, 125.20, 73.28, 65.76, 65.70, 64.26, 64.23, 59.20, 59.15, 54.32, 50.66, 50.61, 36.66, 32.25, 31.88, 29.74, 29.71, 29.70, 29.69, 29.63, 29.60, 29.56, 29.50, 29.46, 29.33, 28.92, 25.79, 22.64, 14.06. HRMS (ESI) m/z [M+H]⁺ for C₄₃H₈₄N₂O₉P calculated 803.5909, found 803.5928.

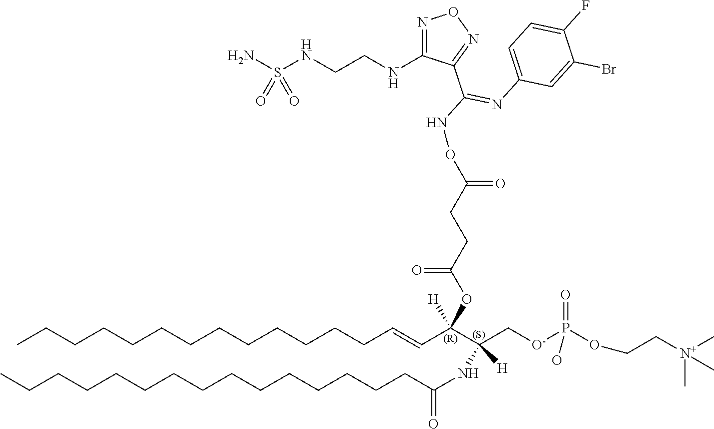

### SM-Ester-EPA

### (2S, 3R, E)-3-((4-(((E)-N′-(3-bromo-4-fluorophenyl)-4-((2-(sulfamoylamino)ethyl)amino)-1,2,5-oxadiazole-3-carboximidamido)oxy)-4-oxobutanoyl)oxy)-2-palmitamidooctadec-4-en-1-yl (2-(trimethylammonio)ethyl) Phosphate

DIPEA (2 mL) was added to a solution of SM-COOH (803.0 mg, 1.0 mmol) and HATU (380 mg, 1.0 mmol) in anhydrous DCM (50 mL). The reaction mixture was stirred at room temperature for 30 min. A solution of EPA (1.0 mmol) in 10 mL anhydrous DCM was added into the reaction and further stirred for 12 h. After completion of the reaction, the reaction mixture was washed with 50 mM HCl aqueous solution, and then with saturated brine. The organic layer was dried with anhydrous Na₂SO₄, the solvent was removed using rotary evaporator under vacuum, and the residue was purified by silica gel flash chromatography with CHCl₃/EtOH/H₂O (v/v/v, 300/200/36) as the eluting solvent. White solid with 56% yield was achieved. R_{f}=0.41 (CHCl₃/EtOH/H₂O=300/200/36). ¹H NMR (400 MHz, DMSO-d₆) δ 10.19 (s, 1H), 7.98 (d, J=8.6 Hz, 1H), 7.49-7.41 (m, 1H), 7.24 (t, J=8.7 Hz, 1H), 7.10-7.03 (m, 1H), 7.00 (d, J=7.8 Hz, 1H), 6.86 (t, J=5.8 Hz, 1H), 6.67 (s, 1H), 5.69-5.62 (m, 1H), 5.35 (dd, J=15.3, 7.6 Hz, 1H), 5.19 (t, J=7.2 Hz, 1H), 4.06 (s, 2H), 3.69 (dd, J=22.0, 15.6 Hz, 3H), 3.51 (d, J=4.2 Hz, 2H), 3.29-3.18 (m, 2H), 3.12 (d, J=6.2 Hz, 9H), 3.07-2.96 (m, 2H), 2.83-2.56 (m, 4H), 2.02 (dd, J=14.4, 7.2 Hz, 2H), 1.94 (d, J=6.5 Hz, 2H), 1.41 (d, J=6.6 Hz, 2H), 1.23 (s, 46H), 0.85 (t, J=6.4 Hz, 6H). ¹³C NMR (101 MHz, CDCl₃) δ 171.40, 171.30, 171.28, 171.22, 171.20, 170.18, 169.82, 169.73, 155.49, 155.44, 155.14, 155.11, 73.93, 73.86, 73.76, 73.45, 67.07, 66.43, 66.42, 66.27, 66.26, 66.25, 66.20, 64.34, 64.33, 64.09, 64.05, 64.02, 59.32, 59.26, 59.21, 56.92, 54.37, 36.69, 32.38, 31.90, 29.72, 29.66, 29.60, 29.52, 29.35, 28.93, 25.82, 22.67, 14.09. HRMS (ESI) m/z [M+Na]⁺ for C₅₄H₉₄BrFN₉O₁₂PS calculated 1246.5540, found 1246.5522.

### Bortezomib-Based Sphingomyelin (SM) Drug Conjugates

Provided below is the synthetic protocol for synthesizing SM drug conjugates featuring bortezomib as the drug.

### Synthesis of SM-B-Bortezomib

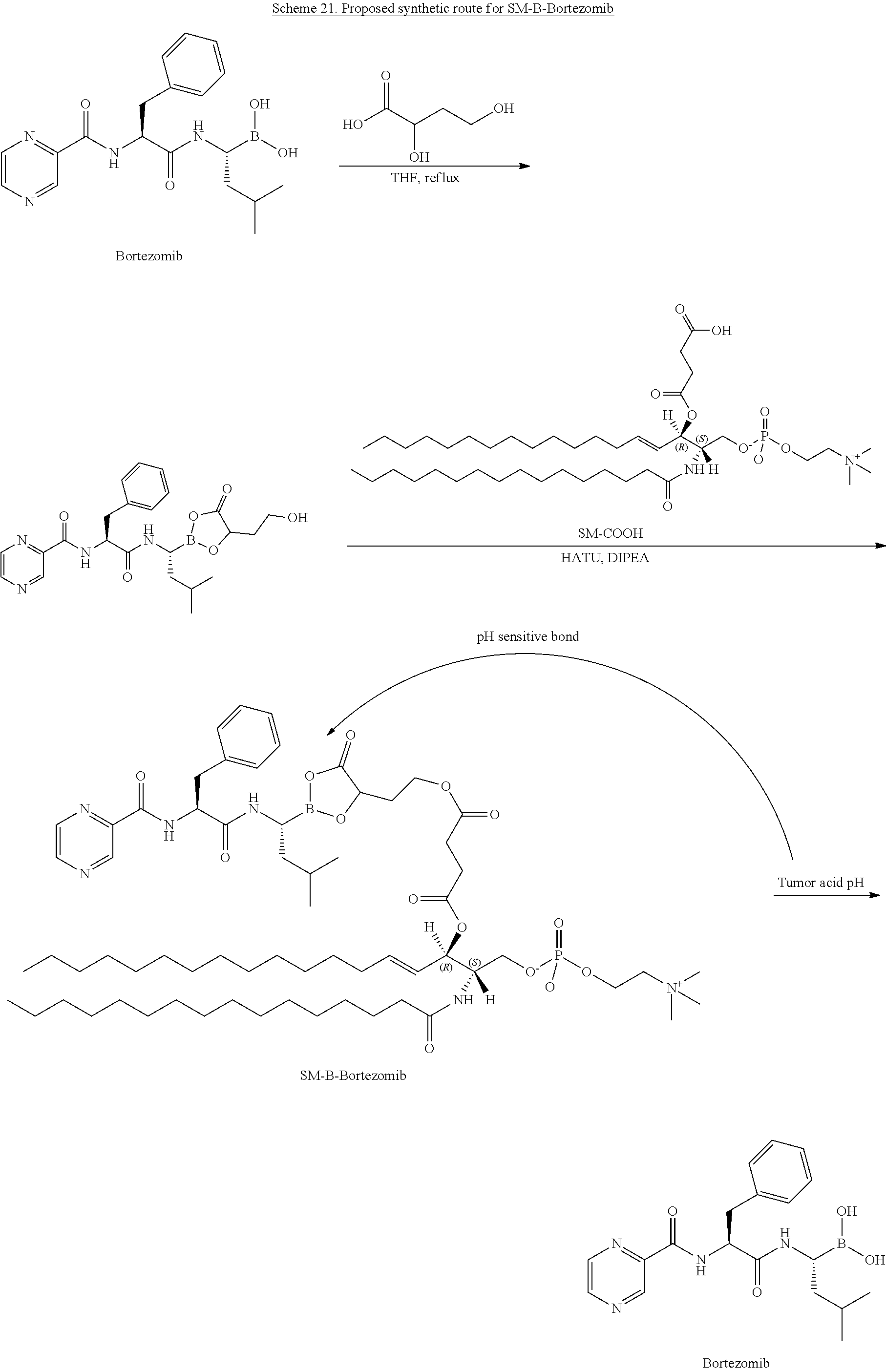

### Imatinib-Based Sphingomyelin (SM) Drug Conjugates

Provided below is the synthetic protocol for synthesizing SM drug conjugates featuring imatinib as the drug.

### Synthesis of SM-CSS-Imatinib

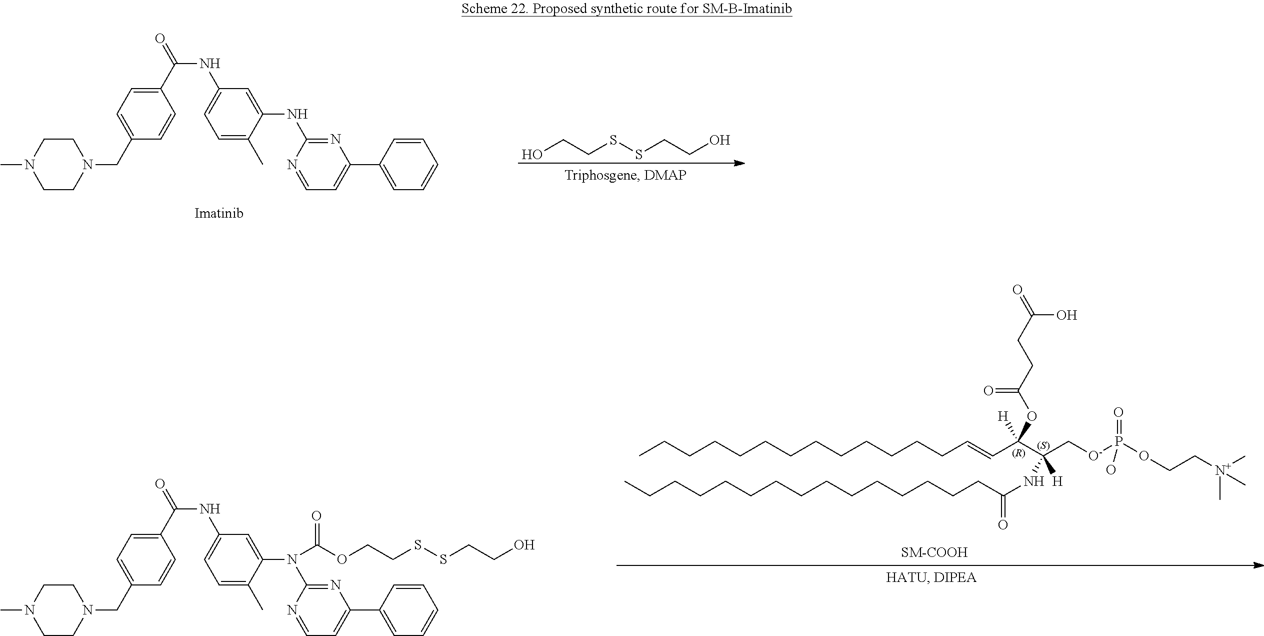

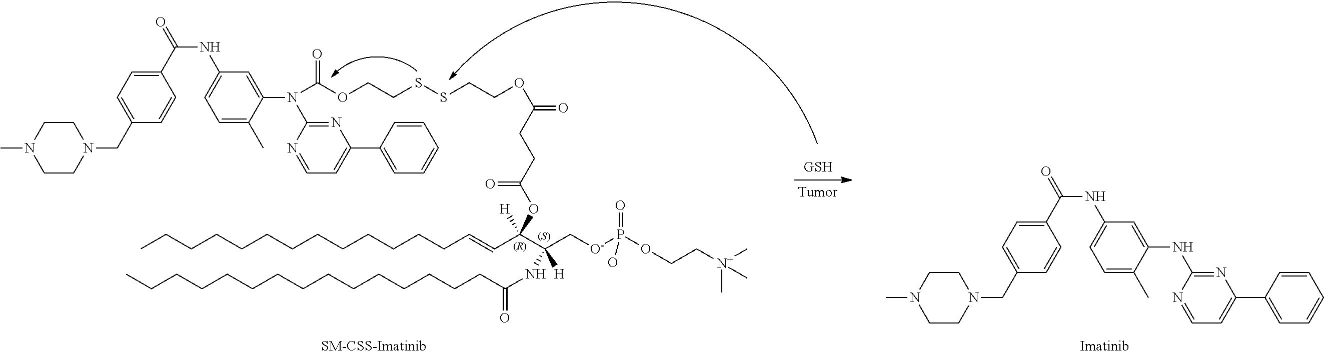

### Canertinib-Based Sphingomyelin (SM) Drug Conjugates

Provided below is the synthetic protocol for synthesizing SM drug conjugates featuring canertinib as the drug.

### Synthesis of SM-CSS-Canertinib

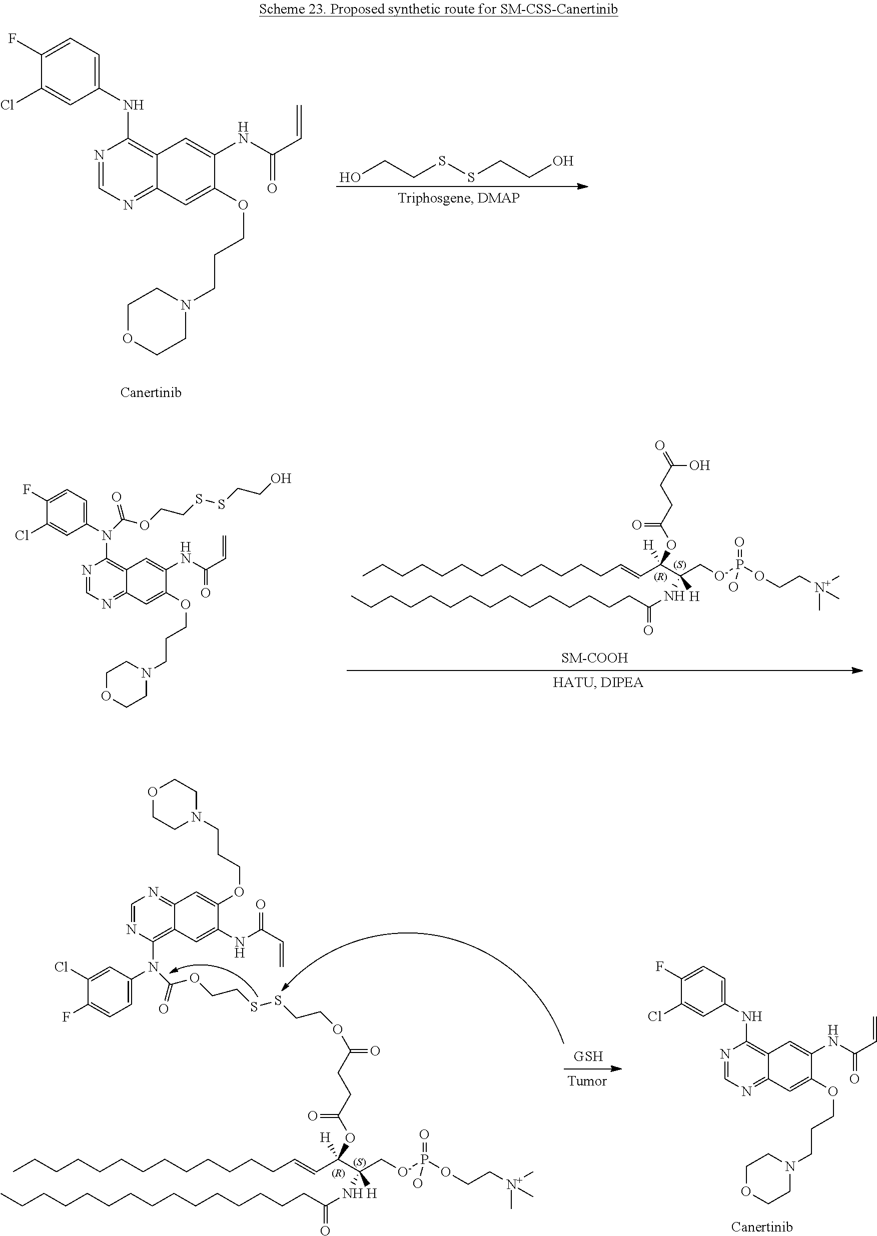

### Ceritinib-Based Sphingomyelin (SM) Drug Conjugates

Provided below is the synthetic protocol for synthesizing SM drug conjugates featuring ceritinib as the drug.

### Synthesis of SM-CSS-Ceritinib

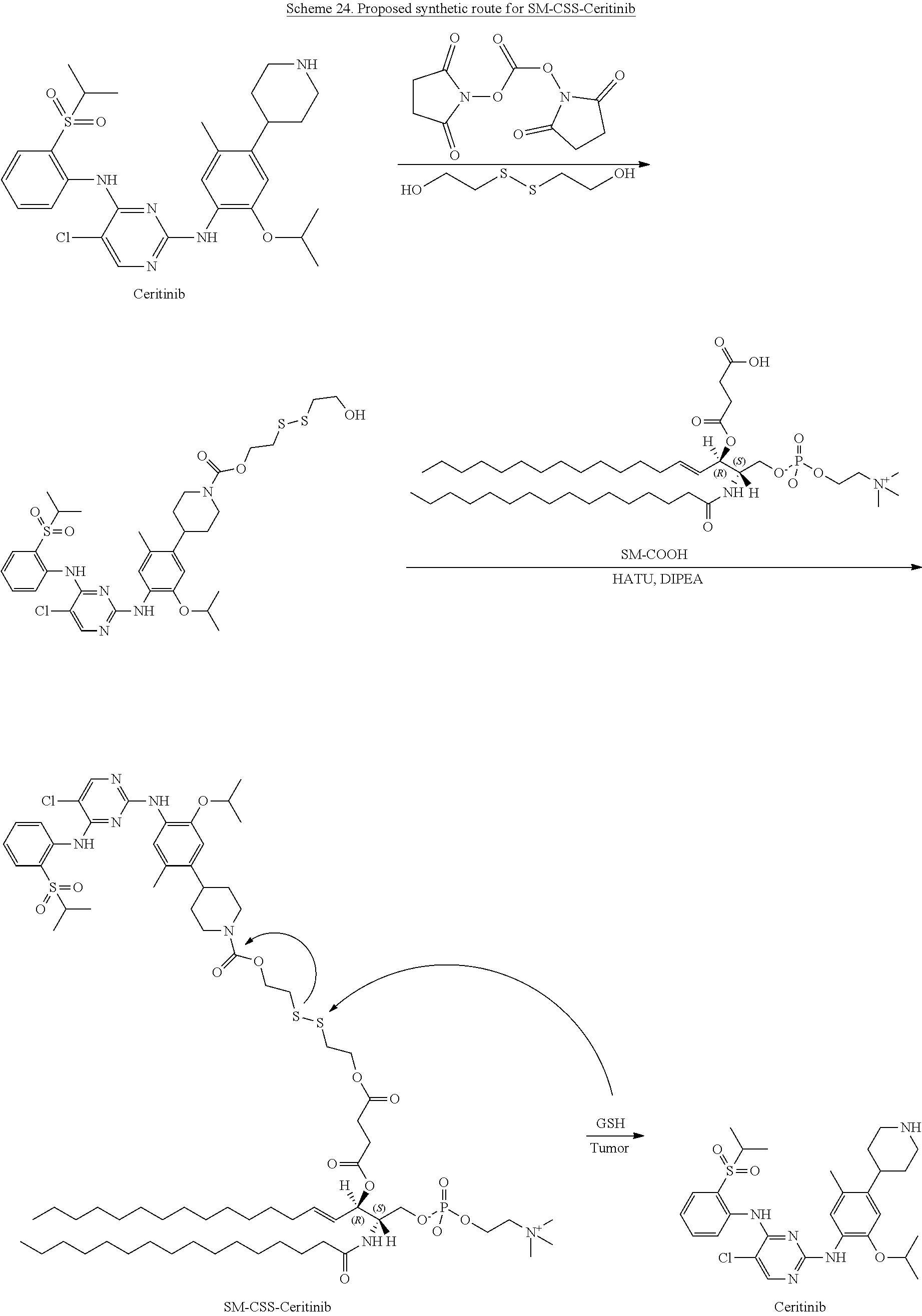

### Dabrafenib-Based Sphingomyelin (SM) Drug Conjugates

Provided below is the synthetic protocol for synthesizing SM drug conjugates featuring dabrafenib as the drug.

### Synthesis of SM-CSS-Dabrafenib

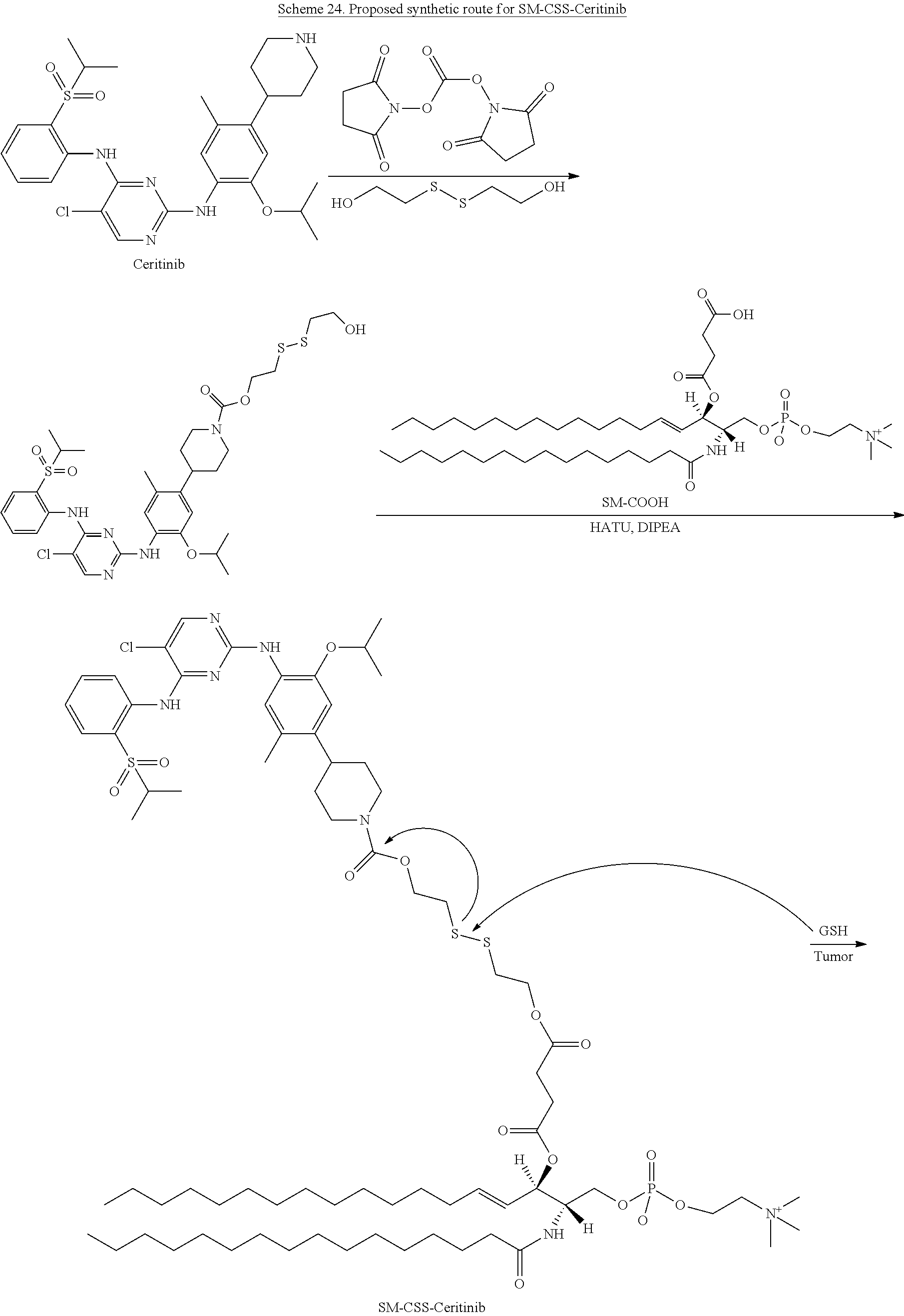

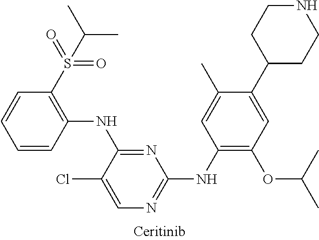 [SMILES: Cc1cc(Nc2ncc(Cl)c(Nc3ccccc3S(=O)(=O)C(C)C)n2)c(OC(C)C)cc1C1CCNCC1]

### Vemurafenib-Based Sphingomyelin (SM) Drug Conjugates

Provided below is the synthetic protocol for synthesizing SM drug conjugates featuring vemurafenib as the drug.

### Synthesis of SM-CSS-Vemurafenib

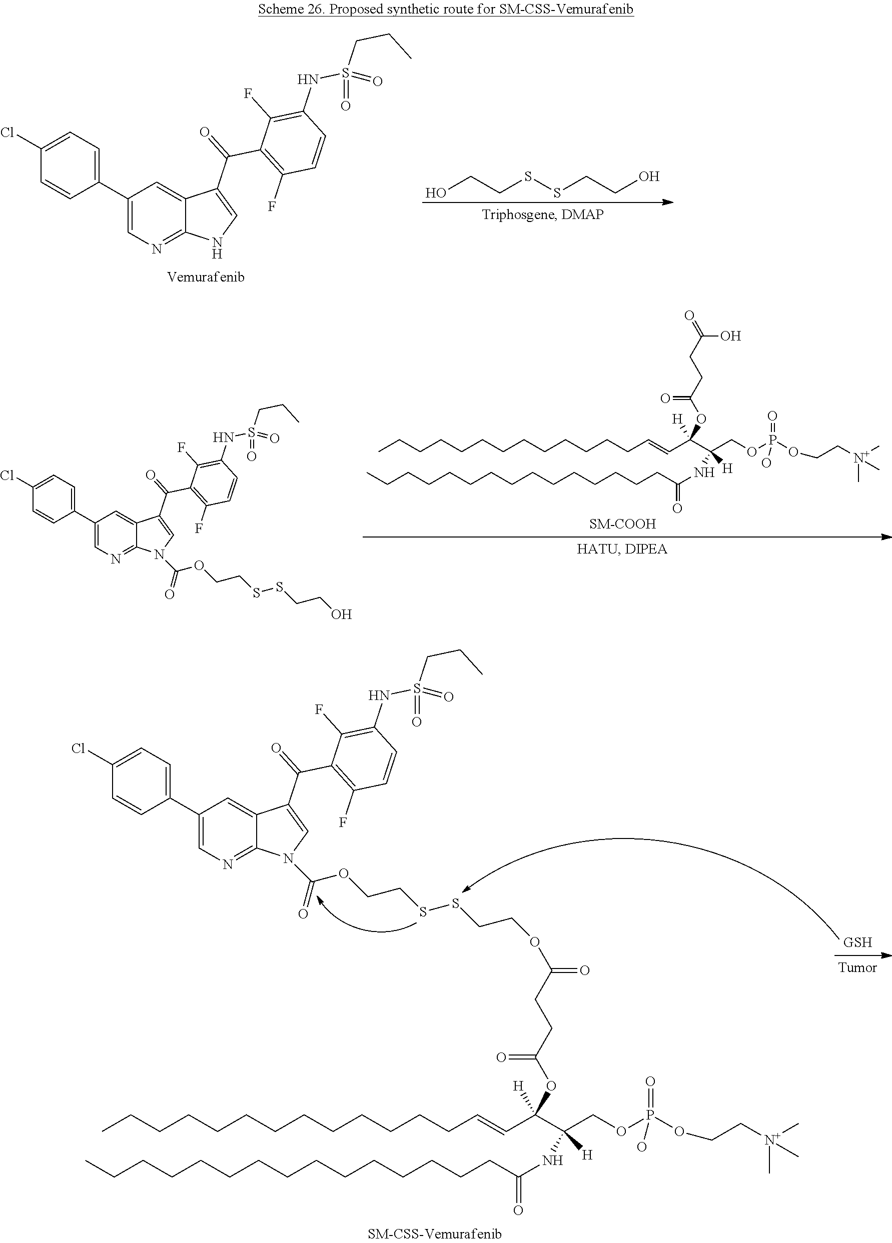 [SMILES: CCCS(=O)(=O)Nc1ccc(F)c(C(=O)c2c[nH]c3ncc(-c4ccc(Cl)cc4)cc23)c1F ; OCCSSCCO ; CCCS(=O)(=O)Nc1ccc(F)c(C(=O)c2cn(C(=O)OCCSSCCO)c3ncc(-c4ccc(Cl)cc4)cc23)c1F ; CCCCCCCCCCCCC/C=C/[C@@H](OC(=O)CCC(=O)O)[C@H](COP(=O)(O)OCC[N+](C)(C)C)NC(=O)CCCCCCCCCCCCCCC ; CCCCCCCCCCCCC/C=C/[C@@H](OC(=O)CCC(=O)OCCSSCCOC(=O)n1cc(C(=O)c2c(F)ccc(NS(=O)(=O)CCC)c2F)c2cc(-c3ccc(Cl)cc3)cnc21)[C@H](COP(=O)(O)OCC[N+](C)(C)C)NC(=O)CCCCCCCCCCCCCCC]

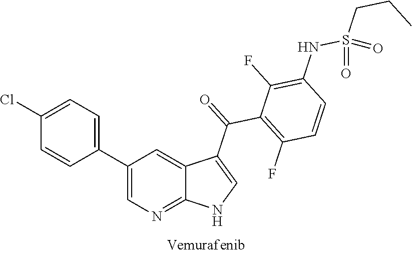 [SMILES: CCCS(=O)(=O)Nc1ccc(F)c(C(=O)c2c[nH]c3ncc(-c4ccc(Cl)cc4)cc23)c1F]

### Oxaliplatin-Based Sphingomyelin (SM) Drug Conjugates

Provided below is the synthetic protocol for synthesizing SM drug conjugates featuring oxaliplatin as the drug.

### Synthesis of SM-CSS-Oxaliplatin

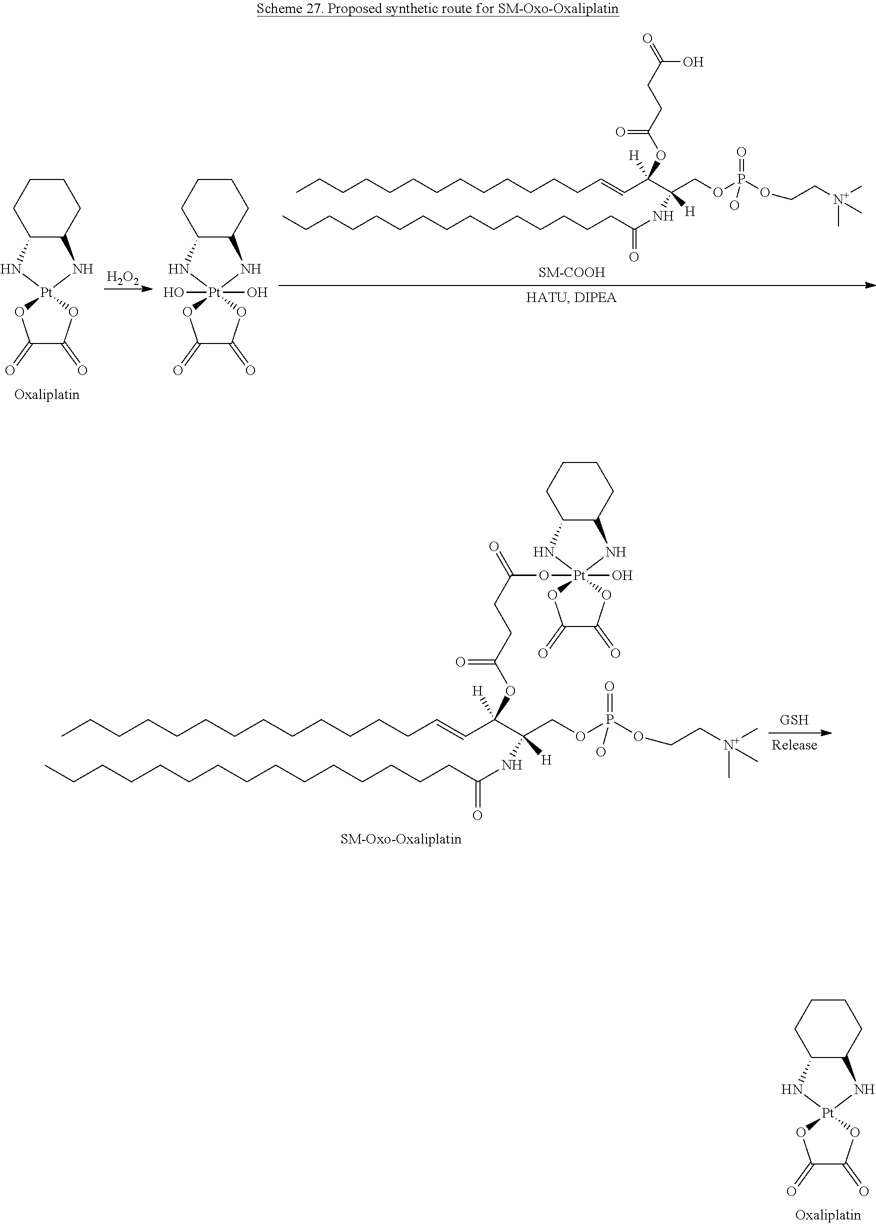

### Vorinostat-Based Sphingomyelin (SM) Drug Conjugates

Provided below is the synthetic protocol for synthesizing SM drug conjugates featuring vorinostat as the drug.

### Synthesis of SM-CSS-Vorinostat

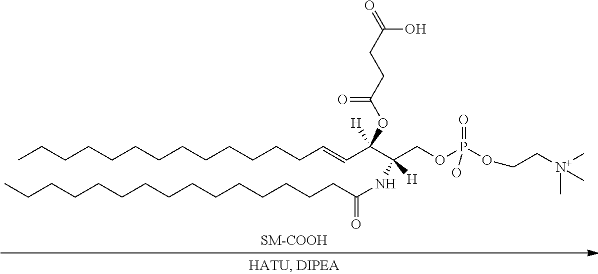 [SMILES: CCCCCCCCCCCCC/C=C/[C@@H](OC(=O)CCC(=O)O)[C@H](COP(=O)(O)OCC[N+](C)(C)C)NC(=O)CCCCCCCCCCCCCCC]

### Preladenant-Based Sphingomyelin (SM) Drug Conjugates

Provided below is the synthetic protocol for synthesizing SM drug conjugates featuring preladenant as the drug.

### Synthesis of SM-CSS-Preladenant

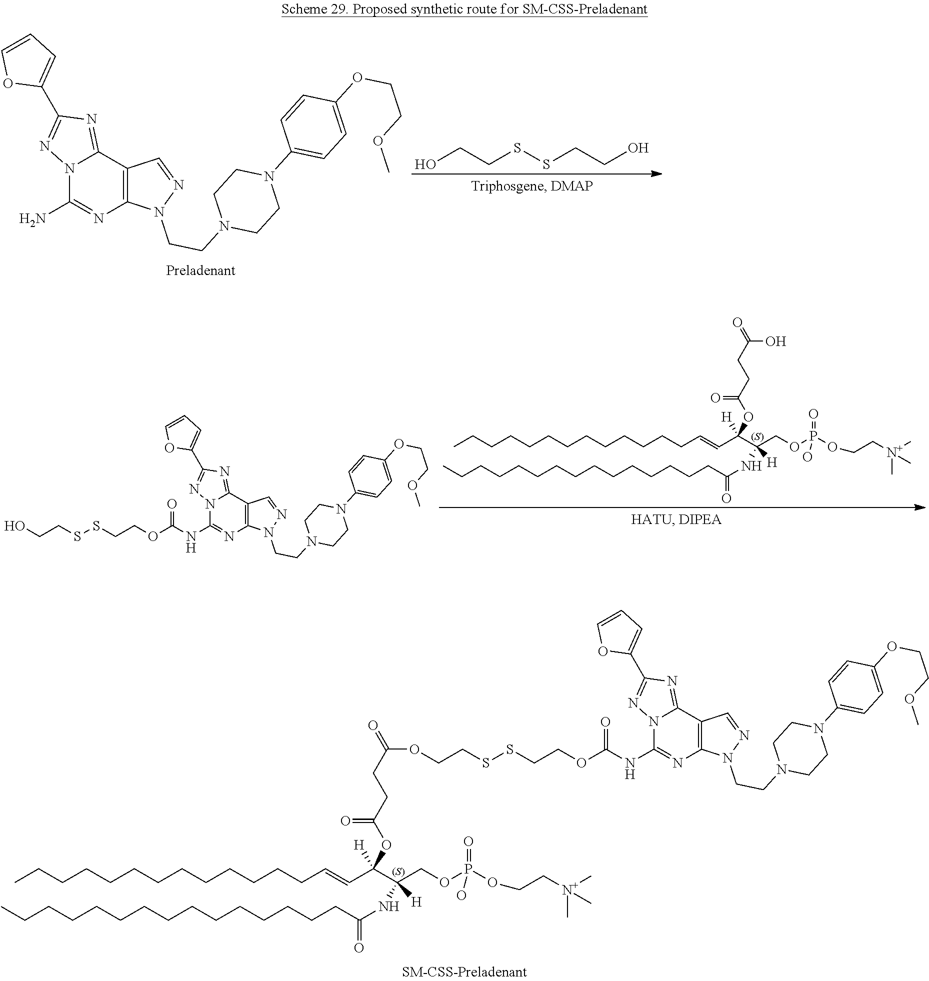

### BMS-1166-Based Sphingomyelin (SM) Drug Conjugates

Provided below is the synthetic protocol for synthesizing SM drug conjugates featuring BMS-1166 as the drug.

### Synthesis of SM-CSS-BMS-1166

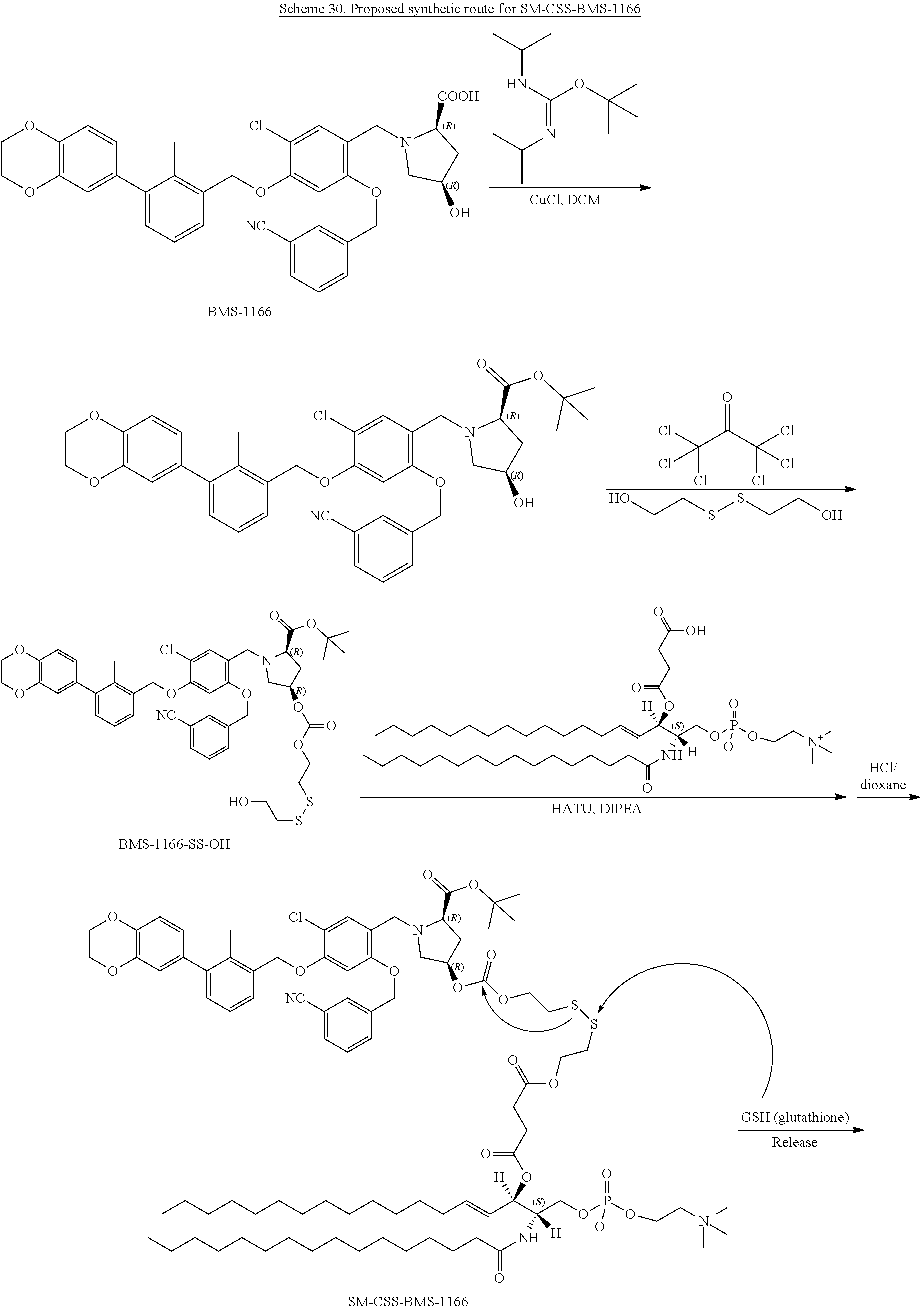

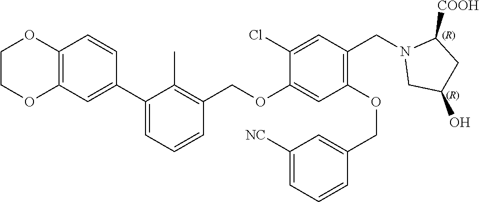

### BMS-1001-Based Sphingomyelin (SM) Drug Conjugates

Provided below is the synthetic protocol for synthesizing SM drug conjugates featuring BMS-1001 as the drug.

### Synthesis of SM-CSS-BMS-1001

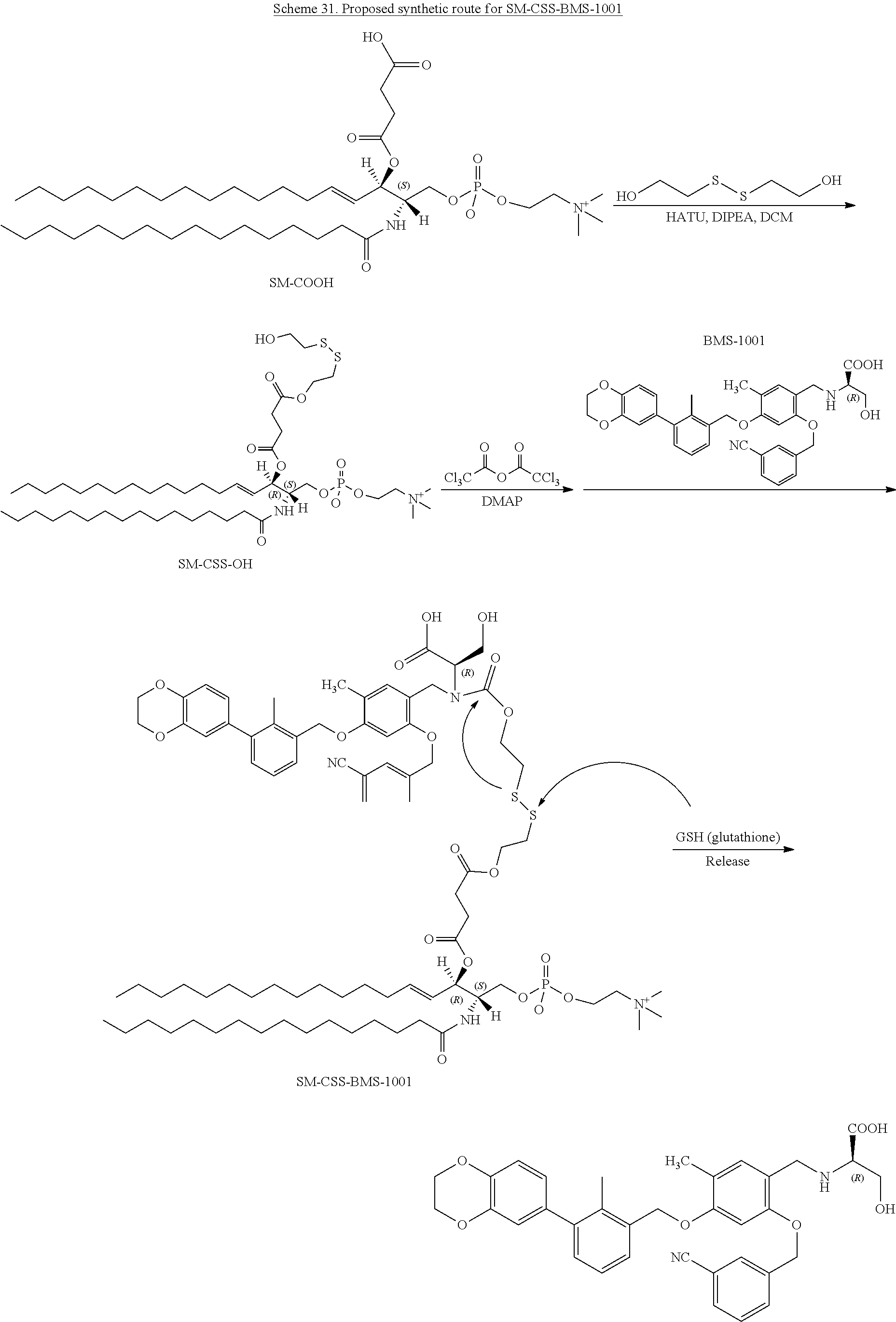

### BMS-200-Based Sphingomyelin (SM) Drug Conjugates

Provided below is the synthetic protocol for synthesizing SM drug conjugates featuring BMS-200 as the drug.

### Synthesis of SM-CSS-BMS-200

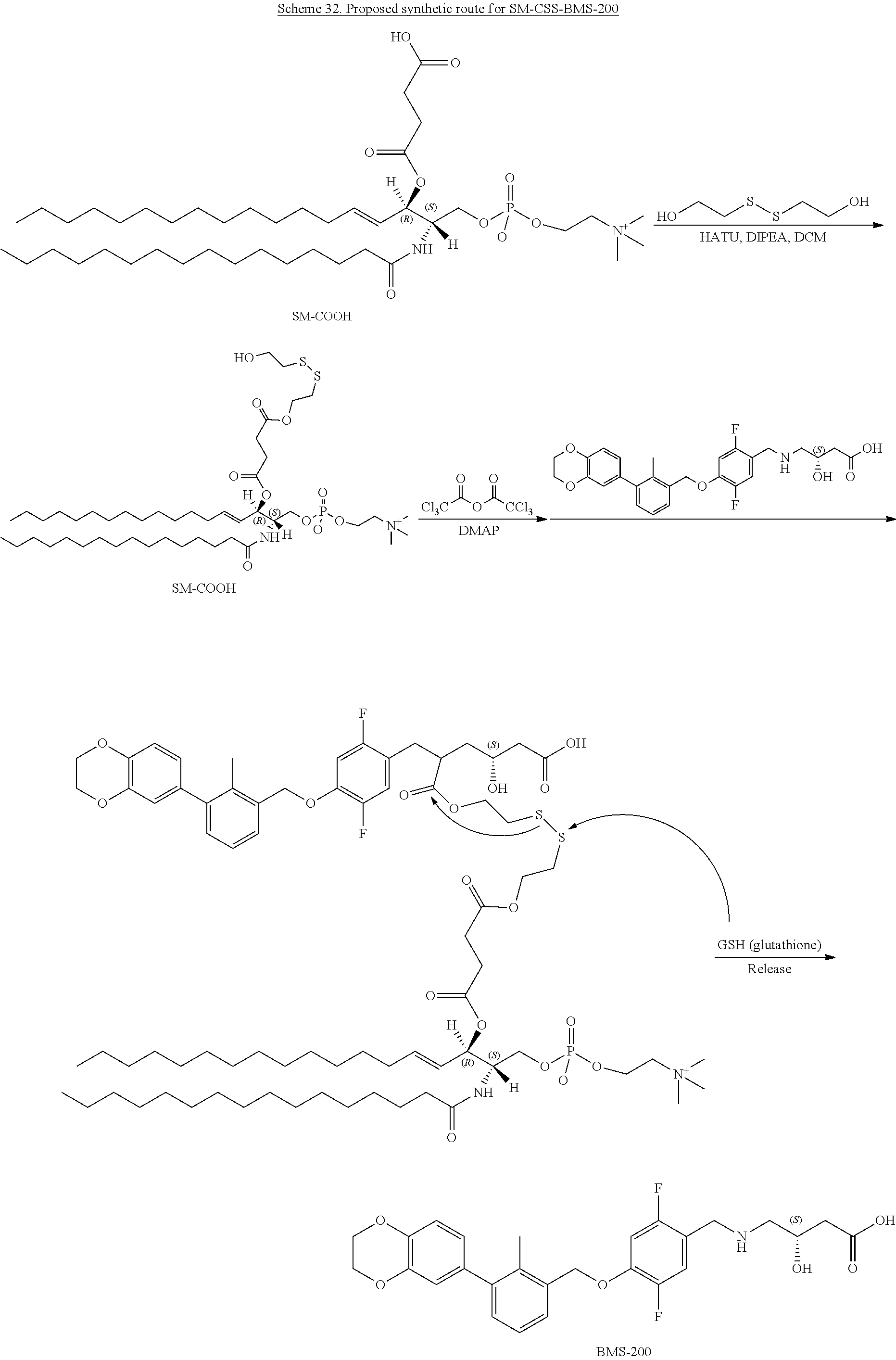

### ADU-S100-Based Sphingomyelin (SM) Drug Conjugates

Provided below is the synthetic protocol for synthesizing SM drug conjugates featuring ADU-S100 as the drug.

### Synthesis of SM-CSS-ADU-S100

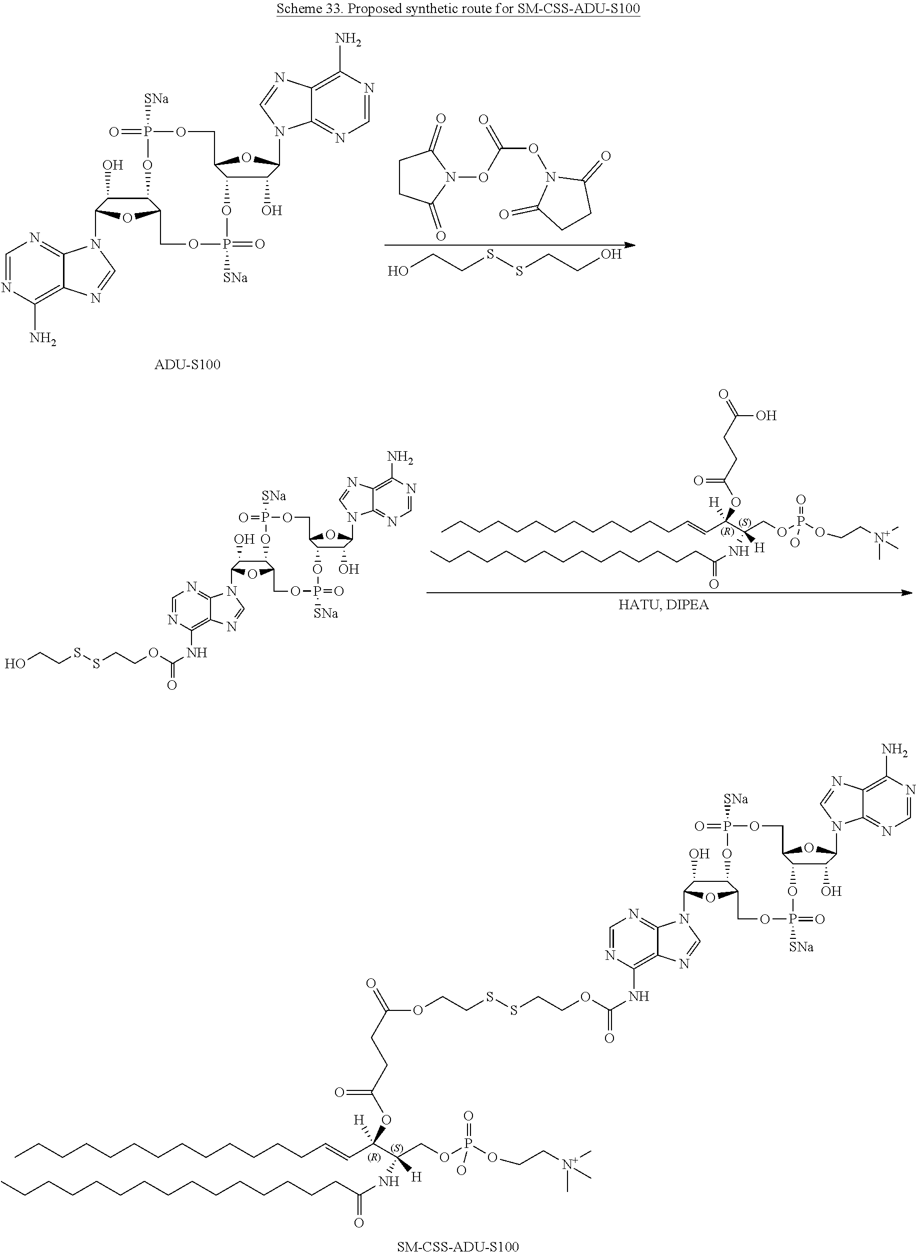

### Vadimezan-Based Sphingomyelin (SM) Drug Conjugates

Provided below is the synthetic protocol for synthesizing SM drug conjugates featuring Vadimezan as the drug.

### Synthesis of SM-CSS-Vadimezan

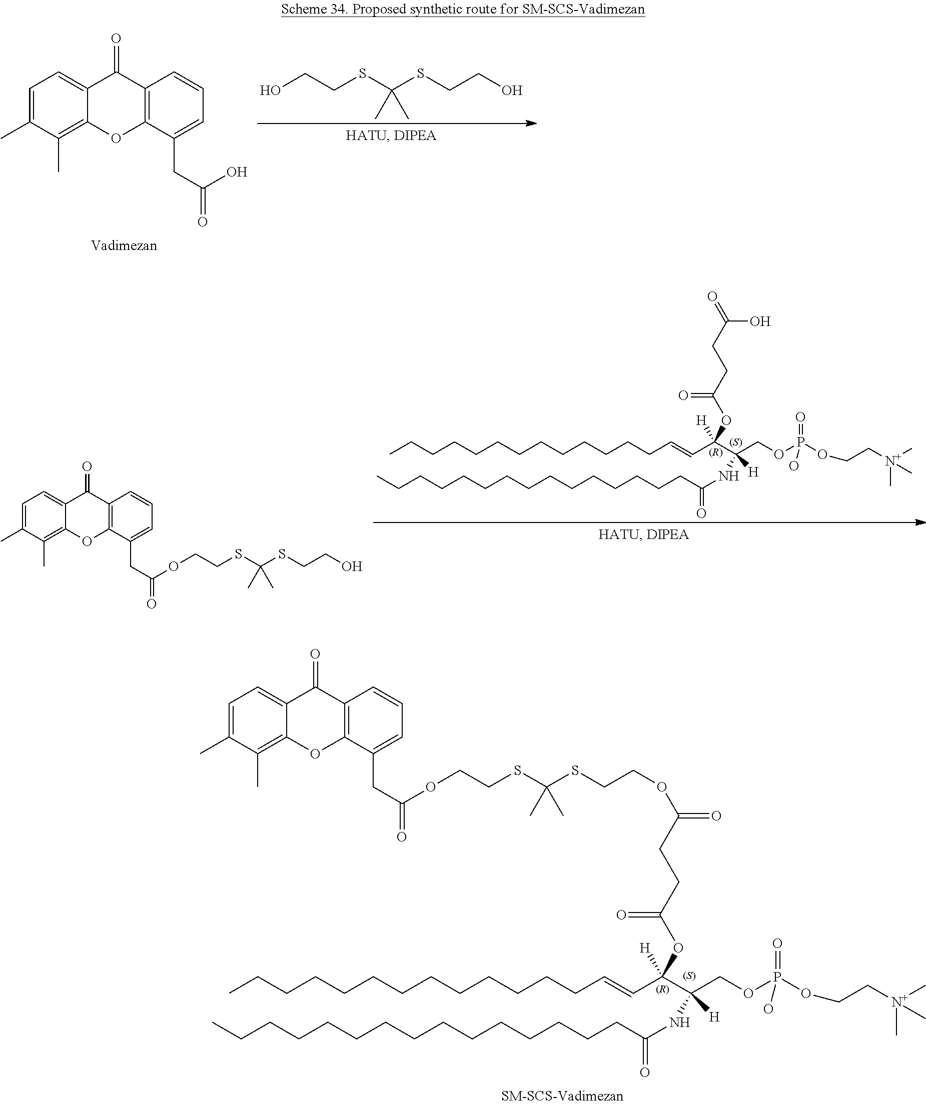

### Pyropheophorbide A-Based Sphingomyelin (SM) Drug Conjugates

Provided below is the synthetic protocol for synthesizing SM drug conjugates featuring Pyropheophorbide A as the drug.

### Synthesis of SM-CSS-Pyropheophorbide A

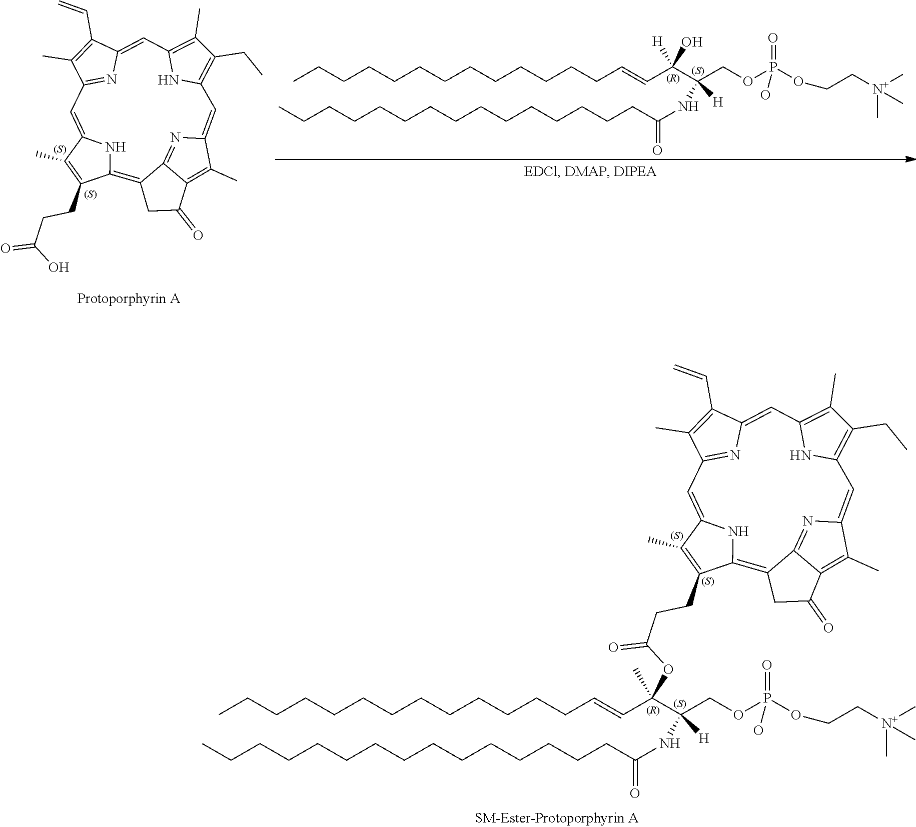

### Protoporphyrin IX-Based Sphingomyelin (SM) Drug Conjugates

Provided below is the synthetic protocol for synthesizing SM drug conjugates featuring Protoporphyrin IX as the drug.

### Synthesis of SM-CSS-Protoporphyrin IX

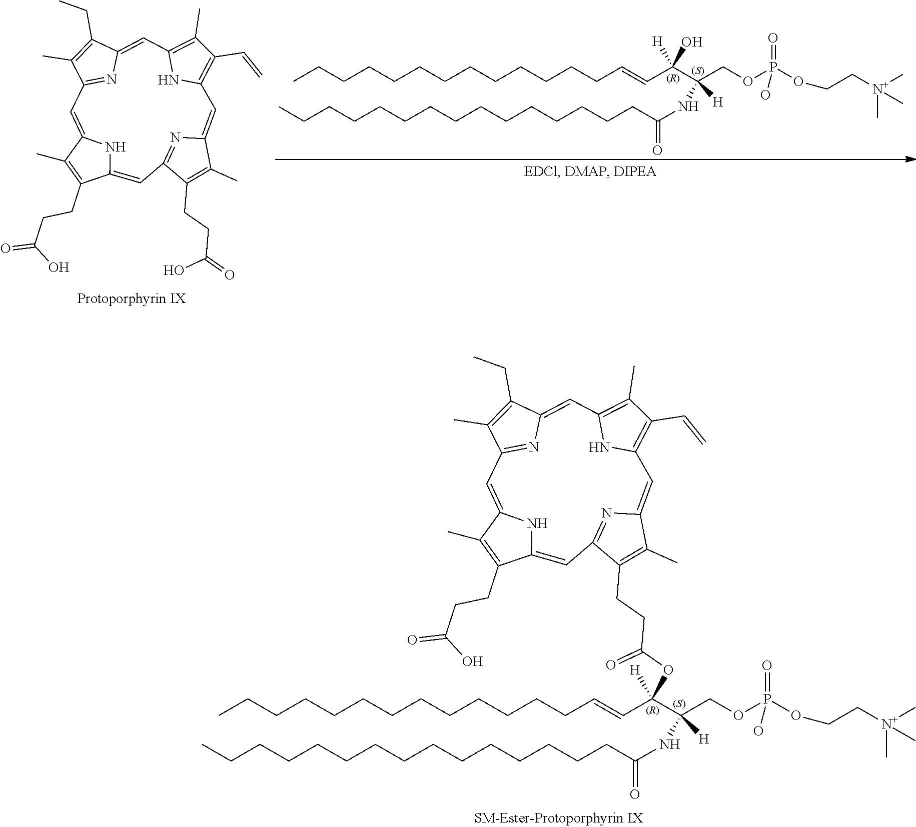

### Lactone Stability Analysis

Camptothecin undergoes a pH-dependent equilibrium between the active lactone and inactive carboxy late form.

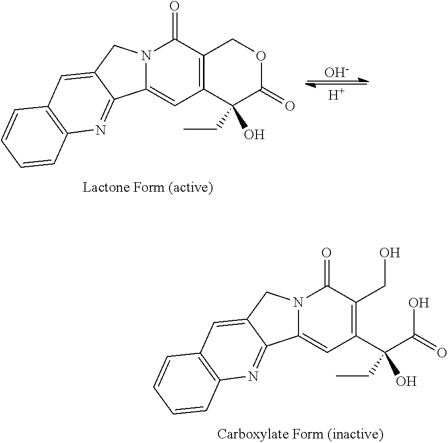

The stability of the lactone form was analyzed using the following method. Stock solutions of 1 mM (in DMSO) CPT and SM-CPT conjugates (n=3) were diluted to 50 μM by PBS (pH 7.4) at 37° C., respectively [12, 53, 54]. At predetermined time points, an aliquot of the sample solutions was analyzed by HPLC/LC-MS (LCMS-2020, SHIMADZU) with an established analytic method. The closed lactone and open carboxylate forms, and CPT intermediate were determined by LC-MS, retention times, and area under the curve based on CPT and SM-CPT conjugates standards. The respective concentrations were calculated by fitting to the standard curve of CPT, CPT-intermediate or SM-CPT conjugates.

### Example 2—DOX-Drug Conjugates

The following example provides representative synthetic protocols and associated synthetic reaction schemes for the synthesis of DOX-drug conjugates of the present disclosure.

### IND-Based DOX-Drug Conjugates

Provided below are the synthetic protocols and proposed synthetic schemes for synthesizing DOX-drug conjugates featuring indoximod (IND) as the drug.

### A. Synthesis of Doxorubicin-Hydrazone-SS-Indoximod (DOX-IND)

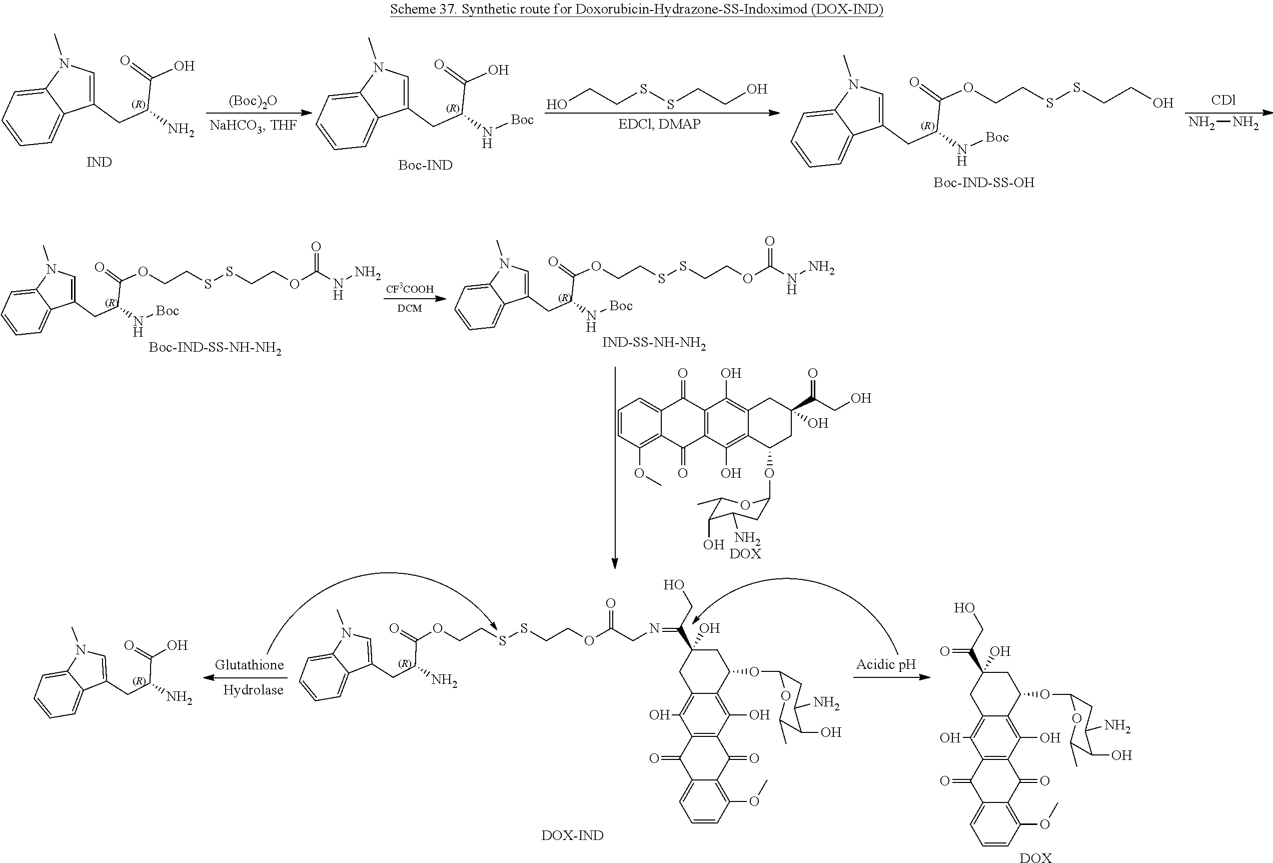

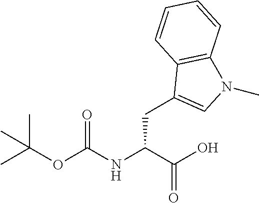 [SMILES: Cn1cc(C[C@@H](NC(=O)OC(C)(C)C)C(=O)O)c2ccccc21]

### Boc-IND

### N^{α}-(tert-butoxycarbonyl)-1-methyl-D-tryptophan [43]

NaHCO₃(2.52 g, 30 mmol) in 50 mL H₂O was added to a solution of 1-methyl-D-tryptophan (Indoximod (IND), 2.18 g, 10 mmol) in THE (50 mL). The solution mixture was stirred in an ice bath for 30 min. Di-tert-butyl decarbonate (2.62 g, 12 mmol) was then added into the reaction mixture, which was stirred at room temperature for 24 h and monitored by TLC. After completion of the reaction, the reaction mixture was adjusted to pH 1 with 1 M HCl aqueous solution, and the product was extracted with EtOAc. The organic phase was then washed with saturated brine and dried with anhydrous Na₂SO₄. The solvent was removed using rotary evaporator under vacuum, and the residue was used for the next step without further purification. White solid with 93% yield was obtained. R_{f}=0.31 (petroleum/EtOAc=1/1). ¹H NMR (400 MHz, CDCl₃) δ 7.62 (d, J=7.9 Hz, 1H), 7.31 (d, J=8.2 Hz, 1H), 7.25 (t, J=7.4 Hz, 1H), 7.14 (dd, J=10.9, 3.9 Hz, 1H), 6.93 (s, 1H), 5.05 (d, J=5.0 Hz, 1H), 4.79-4.52 (m, 1H), 3.76 (s, 3H), 3.48-3.19 (m, 2H), 1.46 (s, 9H). LC/MS (ESI): 319.1 [M+H]⁺.

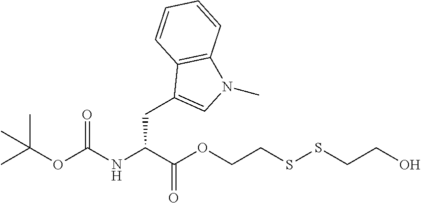 [SMILES: Cn1cc(C[C@@H](NC(=O)OC(C)(C)C)C(=O)OCCSSCCO)c2ccccc21]

### Boc-IND-SS-OH

### 2-((2-hydroxyethyl)disulfanyl)ethyl N^{α}-(tert-butoxycarbonyl)-1-methyl-D-tryptophanate

EDCl (1.84 g, 9.6 mmol) and 6 mL DIPEA was added to a solution of Boc-IND (2.56 g, 8 mmol) in anhydrous DCM. The reaction mixture was stirred at room temperature for 30 min. Then a solution of 2,2′-disulfanediylbis(ethan-1-ol) (4.88 g, 32 mmol) in 20 mL TH F was added into the mixture solution followed by addition of DMAP (98 mg, 0.8 mmol). The reaction solution was further stirred at room temperature for 12 h and monitored by TLC. After completion of the reaction, the solution mixture was washed with 50 mM HCl aqueous solution and then with saturated brine. The organic layer was dried with anhydrous Na₂SO₄, the solvent was evaporated using rotary evaporator under vacuum, and the residue was purified by silica gel flash chromatography. White solid with 85% yield was attained. R_{f}=0.32 (petroleum/EtOAc=3/1). ¹H NMR (400 MHz, CDCl₃) δ 7.52 (d, J=7.9 Hz, 1H), 7.27 (d, J=8.2 Hz, 1H), 7.22-7.16 (m, 1H), 7.09 (t, J=7.4 Hz, 1H), 6.88 (s, 1H), 5.04 (d, J=7.7 Hz, 1H), 4.60 (d, J=6.5 Hz, 1H), 4.29 (ddd, J=17.7, 11.8, 6.2 Hz, 2H), 3.81 (d, J=5.4 Hz, 2H), 3.74 (s, 3H), 3.25 (d, J=5.2 Hz, 2H), 2.86-2.62 (m, 4H), 1.41 (s, 9H). LC/MS (ESI): 455.1 [M+H]⁺.

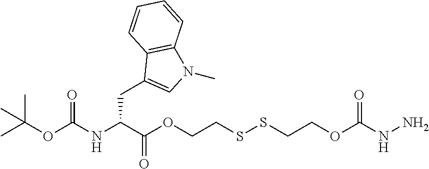 [SMILES: Cn1cc(C[C@@H](NC(=O)OC(C)(C)C)C(=O)OCCSSCCOC(=O)NN)c2ccccc21]

### Boc-IND-SS—NH—NH₂

### (R)-13,13-dim ethyl-9-((1-m ethyl-1H-indol-3-yl)methyl)-8,11-dioxo-7,12-dioxa-3,4-dithia-10-azatetradecyl hydrazinecarboxylate

CDI (0.98 g, 6 mmol) was added to a solution of Boc-IND-SS-OH (2.27 g, 5 mmol) in anhydrous DCM (50 mL). The reaction mixture was stirred at room temperature for 30 min and monitored by TLC until the formation of imidazolide was completed. Then hydrazine hydrate (0.5 mL) was added into reaction mixture and the solution was stirred for 2 h. The reaction mixture was monitored by TLC. After completion of the reaction, the solution mixture was washed with H₂O and then with saturated brine. The organic layer was dried with anhydrous Na₂SO₄, the solvent was evaporated using rotary evaporator under vacuum, and the residue was purified by silica gel flash chromatography. White solid with 82% yield was acquired. R_{f}=0.28 (petroleum/EtOAc=2/1). ¹H NMR (400 MHz, CDCl₃) δ 7.53 (d, J=7.9 Hz, 1H), 7.27 (d, J=8.2 Hz, 1H), 7.20 (t, J=7.5 Hz, 1H), 7.09 (t, J=7.1 Hz, 1H), 6.87 (s, 1H), 6.29 (s, 1H), 5.17 (d, J=7.5 Hz, 1H), 4.62 (d, J=6.3 Hz, 1H), 4.29 (t, J=5.9 Hz, 4H), 3.72 (d, J=6.5 Hz, 5H), 3.25 (d, J=5.0 Hz, 2H), 2.87 (t, J=6.2 Hz, 2H), 2.82-2.68 (m, 2H), 1.41 (s, 9H). LC/MS (ESI): 513.1 [M+H]⁺.

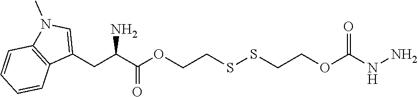 [SMILES: Cn1cc(C[C@@H](N)C(=O)OCCSSCCOC(=O)NN)c2ccccc21]

### Ind-Ss-Nh-Nh₂

### 2-((2-((1-methyl-D-tryptophyl)oxy)ethyl)disulfanyl)ethyl Hydrazinecarboxylate

Anisole (10 mL) was added to a solution of Boc-IND-SS—NH—NH₂ (2.563 g, 4 mmol) in anhydrous DCM (50 mL) followed by 2 m CF₃COOH addition. The reaction mixture was stirred at room temperature for 2 h and monitored by TLC. After completion of the reaction, the solvent was evaporated using rotary evaporator under vacuum, the mixture residue was dissolved in DCM and washed with saturated NaHCO₃aqueous solution and then with saturated brine. The organic layer was dried with anhydrous Na₂SO₄, and the residue was purified by silica gel flash chromatography. Pale yellow solid with 62% yield was obtained. R_{f}=0.26 (CH₂Cl₂/CH₃OH=10/1). ¹H NMR (400 MHz, CDCl₃) δ 7.59 (t, J=9.7 Hz, 1H), 7.30 (d, J=8.1 Hz, 1H), 7.25-7.20 (m, 1H), 7.11 (t, J=7.0 Hz, 1H), 6.95 (s, 1H), 6.23 (s, 1H), 4.36 (dd, J=12.7, 6.3 Hz, 5H), 4.01-3.78 (m, 2H), 3.76 (s, 3H), 3.27 (dd, J=14.3, 4.9 Hz, 1H), 3.05 (dd, J=14.3, 7.6 Hz, 1H), 2.94-2.89 (m, 2H), 2.88-2.81 (m, 4H). HRMS (ESI) m/z [M+H]⁺ for C₁₇H₂₅N₄O₄S₂ calculated 413.1312, found 413.1307.

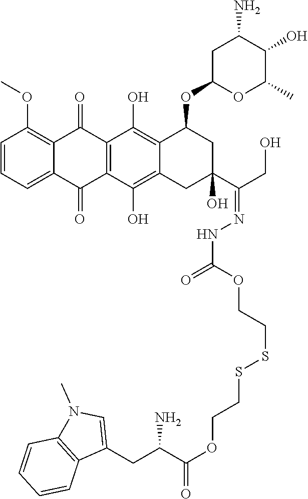 [SMILES: COc1cccc2c1C(=O)c1c(O)c3c(c(O)c1C2=O)C[C@@](O)(/C(CO)=N\NC(=O)OCCSSCCOC(=O)[C@@H](N)Cc1cn(C)c2ccccc12)C[C@@H]3O[C@H]1C[C@H](N)[C@H](O)[C@H](C)O1]

### Doxorubicin-Hydrazone-SS-Indoximod (DOX-IND)

### 2-((2-((1-methyl-D-tryptophyl)oxy)ethyl)disulfanyl)ethyl (Z)-2-(1-((2S,4S)-4-(((2R,4S, 5S, 6S)-4-amino-5-hydroxy-6-m ethyltetrahydro-2H-pyran-2-yl)oxy)-2,5,12-trihydroxy-7-m ethoxy-6,11-dioxo-1,2,3,4,6,11-hexahydrotetracen-2-yl)-2-hydroxyethylidene)hydrazine-1-carboxylate

10 μL CF₃COOH was added to a solution of IND-SS—NH—NH₂ (412 mg, 1 mmol) and doxorubicin hydrochloride (580 mg, 1 mmol) in 20 mL anhydrous CH₃OH. The solution mixture was stirred at room temperature for 36 h and monitored by TLC. After completion of the reaction, the suspension solution was placed under an ultrasonic bath for 10 min, the precipitation was filtered through a suction filtration. The solid was further mixed in 20 m anhydrous acetonitrile and placed under an ultrasonic bath for 10 min, filtered, the product was obtained by repeating this procedure two times. Brick red solid with 47% yield was obtained. R_{f}=0.37 (CHCl₃/EtOH/H₂O=300/200/36). ¹H NMR (400 MHz, DMSO-d₆) δ 7.93-7.86 (m, 2H), 7.69-7.58 (m, 2H), 7.43 (d, J=7.5 Hz, 1H), 7.28 (d, J=7.9 Hz, 1H), 7.04 (t, J=7.3 Hz, 1H), 6.98-6.88 (m, 1H), 5.45-5.33 (m, 2H), 5.27 (s, 1H), 5.13 (s, 1H), 5.05 (s, 1H), 4.99-4.89 (m, 1H), 4.83 (t, J=5.6 Hz, 1H), 4.53 (d, J=5.7 Hz, 1H), 4.47-4.32 (m, 2H), 4.31-4.20 (m, 2H), 4.13 (dd, J=14.0, 10.0 Hz, 2H), 3.95 (d, J=5.4 Hz, 3H), 3.66 (s, 3H), 3.50 (s, 1H), 3.44-3.36 (m, 1H), 3.14 (d, J=17.2 Hz, 2H), 3.01-2.85 (m, 4H), 2.84-2.71 (m, 2H), 2.63 (s, 1H), 2.29 (s, 1H), 2.12 (s, 1H), 1.87-1.81 (m, 1H), 1.65 (dd, J=9.4, 5.8 Hz, 1H), 1.13 (t, J=6.0 Hz, 3H), 1.02 (t, J=7.0 Hz, 1H). ¹³C NMR (101 MHz, DMSO-d₆) δ 173.35, 170.94, 158.49, 156.19, 137.05, 136.98, 129.60, 128.97, 128.08, 127.99, 121.57, 119.34, 118.97, 118.86, 110.04, 109.93, 108.34, 107.66, 99.70, 66.59, 66.45, 64.39, 63.20, 62.99, 62.81, 62.36, 60.00, 59.89, 56.99, 56.47, 55.42, 54.68, 52.94, 52.55, 47.03, 45.74, 41.58, 41.53, 37.58, 37.31, 36.56, 32.82, 32.78, 28.69, 23.08, 19.01, 17.24, 9.02, 7.73. HRMS (ESI) m/z [M+H]⁺ for C₄₄H₅₁N₅O₁₄S₂ calculated 938.2947, found 938.2935; HPLC purity: 98.3%, retention time: 9.667 min.

### B. Synthetic Scheme for the Synthesis of Doxorubicin-GFLG-IND

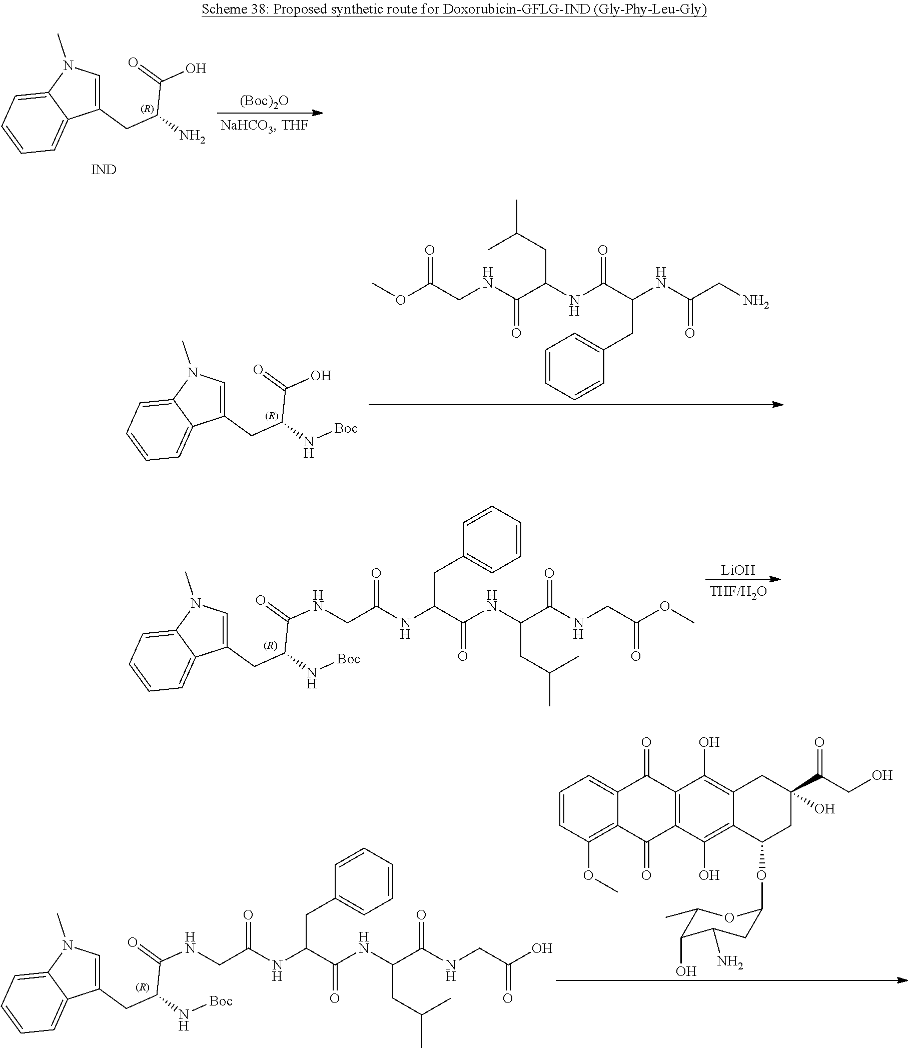

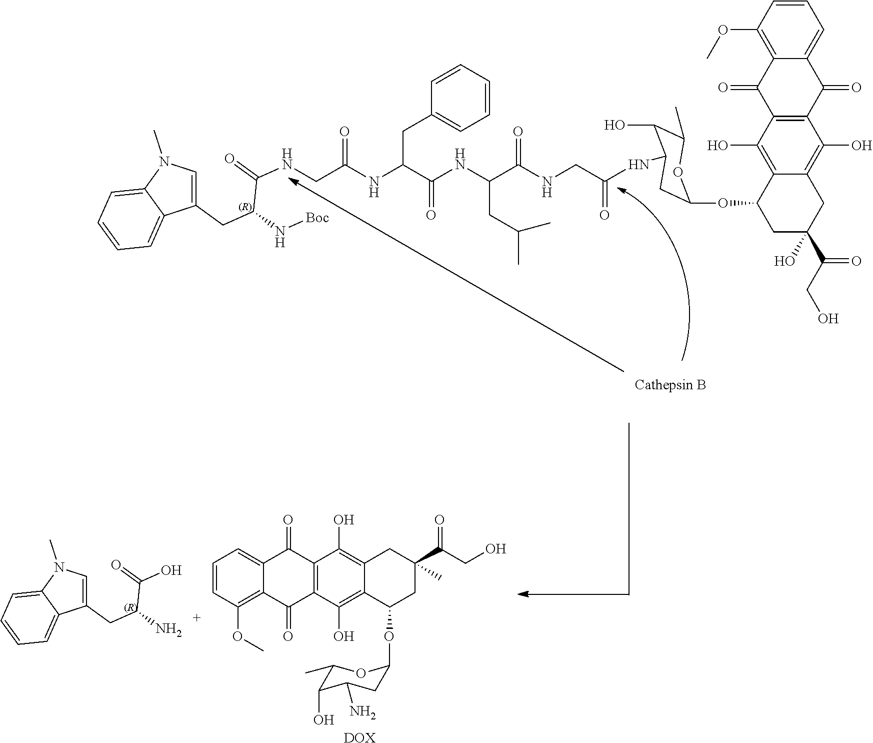

### C. Synthetic Scheme for the Synthesis of Doxorubicin-DMM-IND

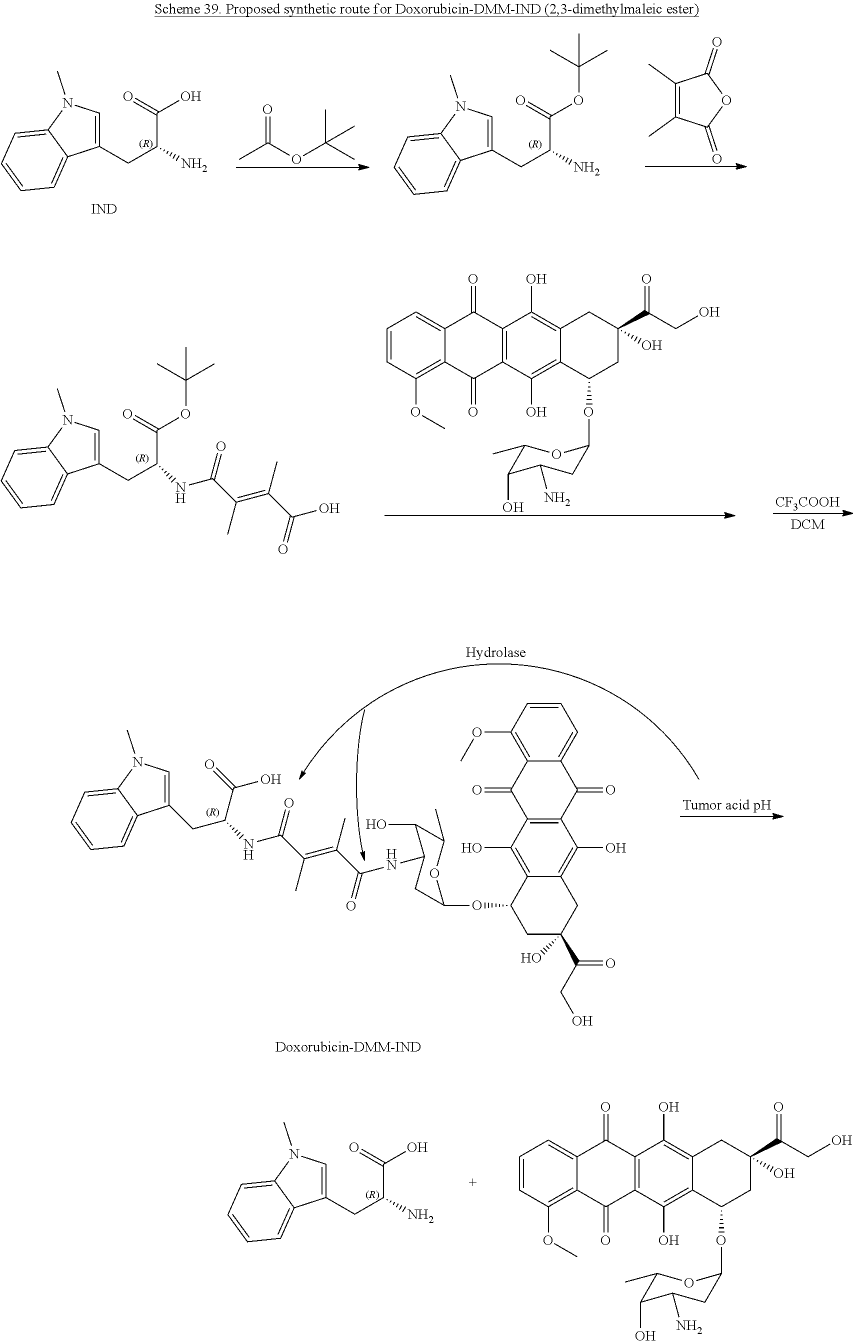

### D. Synthetic Scheme for the Synthesis of Doxorubicin-AANK-IND

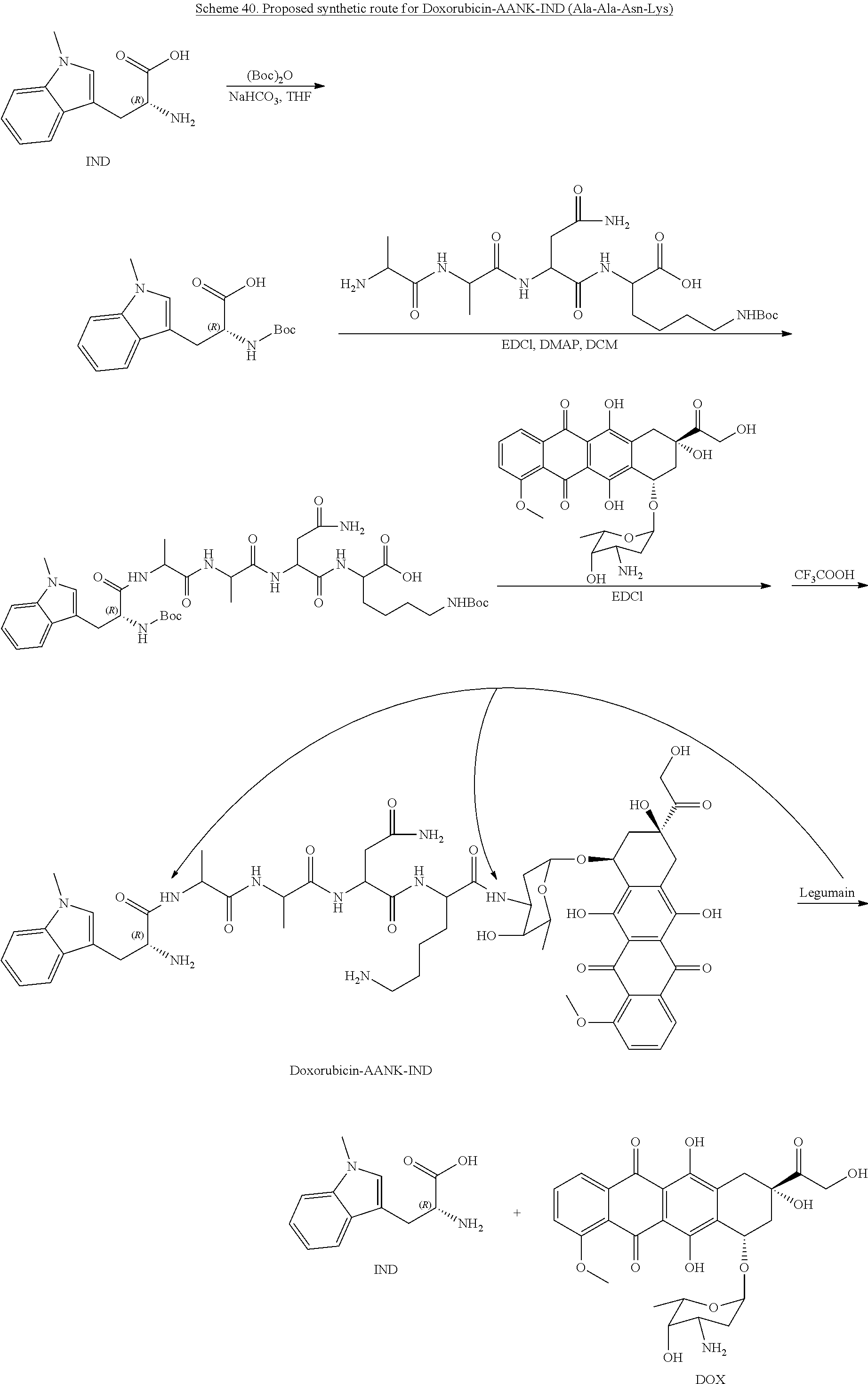

### Bortezomib-Based DOX-Drug Conjugates

Provided below is the synthetic protocol for synthesizing DOX-drug conjugates featuring bortezomib as the drug.

### Synthetic Scheme for the Synthesis of Doxorubicin-DMM-Bortezomib

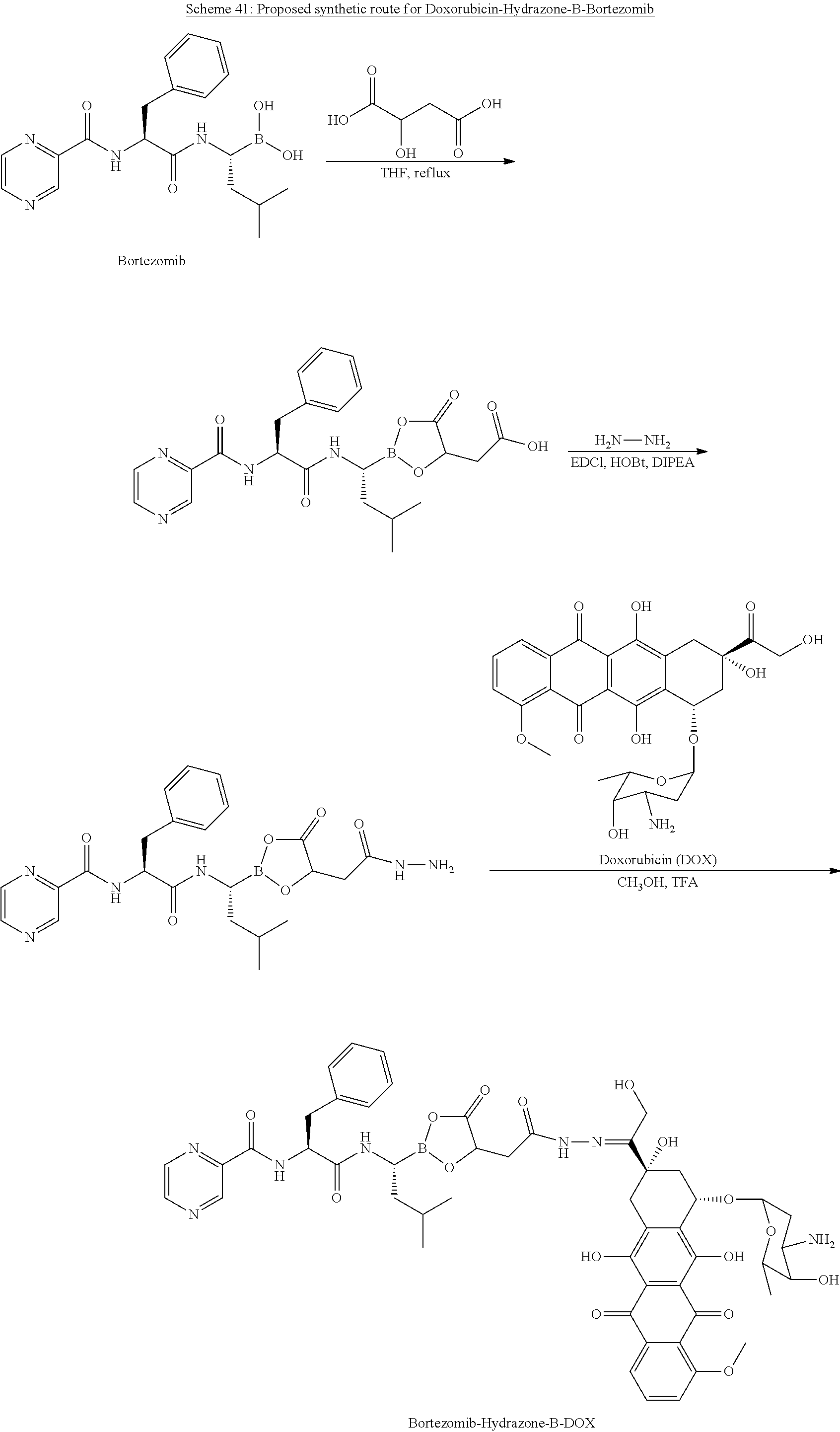

### Epacadostat-Based DOX-Drug Conjugates

Provided below is the synthetic protocol for synthesizing DOX-drug conjugates featuring epacadostat as the drug.

### Synthetic Scheme for the Synthesis of Doxorubicin-Hydrazone-Ester-Epacadostat

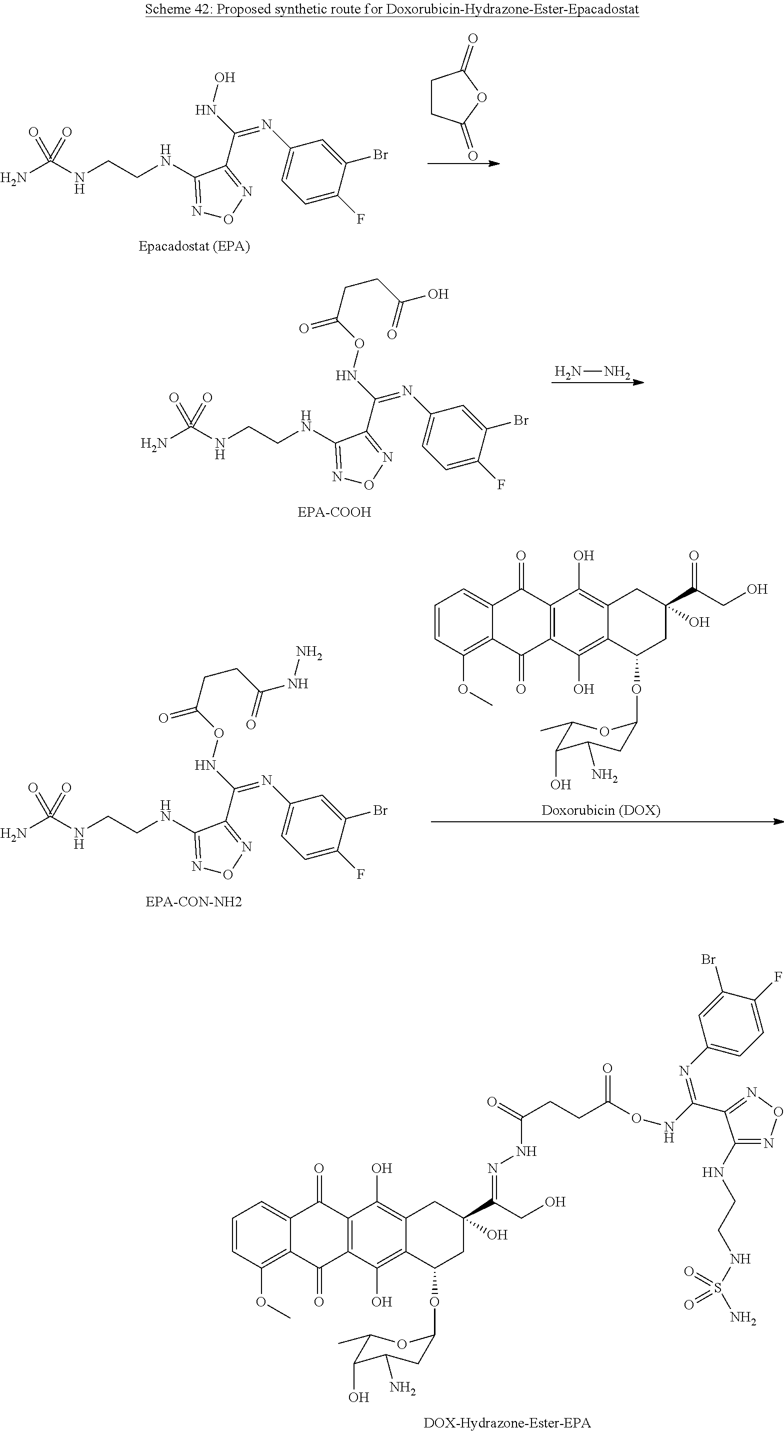

### Imiquimod-Based DOX-Drug Conjugates

Provided below is the synthetic protocol for synthesizing DOX-drug conjugates featuring imiquimod as the drug.

### Synthetic Scheme for the Synthesis of Doxorubicin-SS-Imiquimod

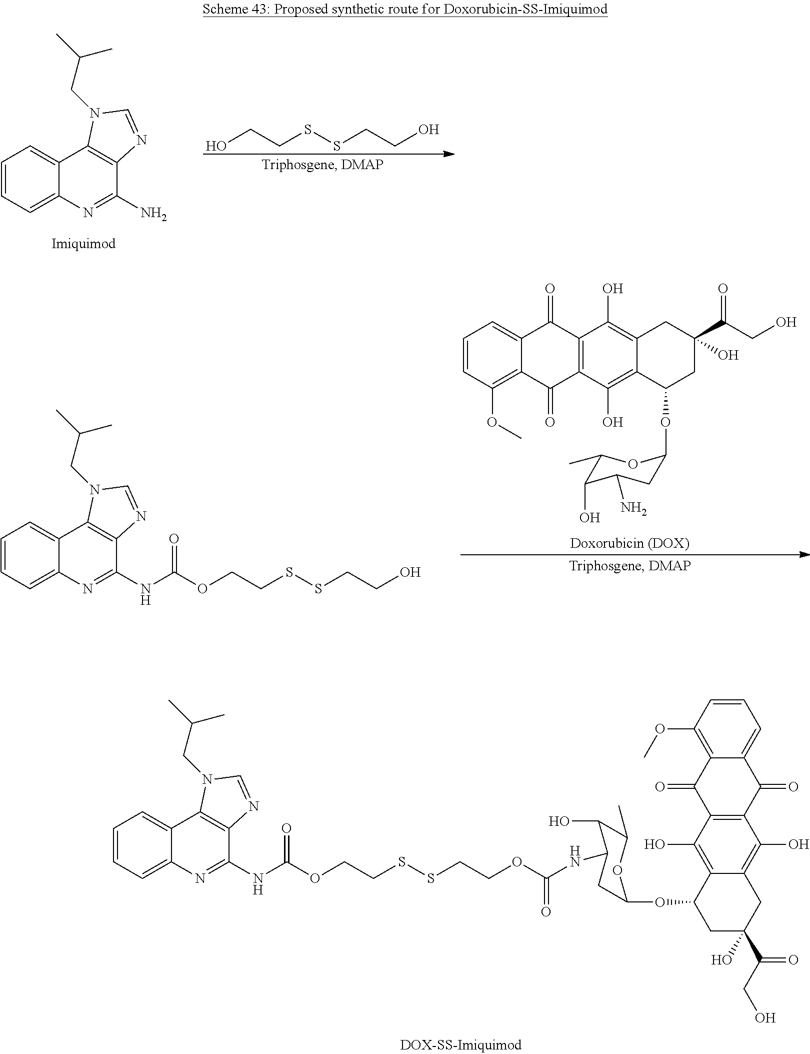 [SMILES: CC(C)Cn1cnc2c(N)nc3ccccc3c21 ; OCCSSCCO ; CC(C)Cn1cnc2c(NC(=O)OCCSSCCO)nc3ccccc3c21 ; COc1cccc2c1C(=O)c1c(O)c3c(c(O)c1C2=O)C[C@@](O)(C(=O)CO)C[C@@H]3OC1CC(N)C(O)C(C)O1 ; COc1cccc2c1C(=O)c1c(O)c3c(c(O)c1C2=O)C[C@@](O)(C(=O)CO)C[C@@H]3OC1CC(NC(=O)OCCSSCCOC(=O)Nc2nc3ccccc3c3c2ncn3CC(C)C)C(O)C(C)O1]

### Imatinib-Based DOX-Drug Conjugates

Provided below is the synthetic protocol for synthesizing DOX-drug conjugates featuring imatinib as the drug.

### Synthetic Scheme for the Synthesis of Doxorubicin-SS-Imatinib

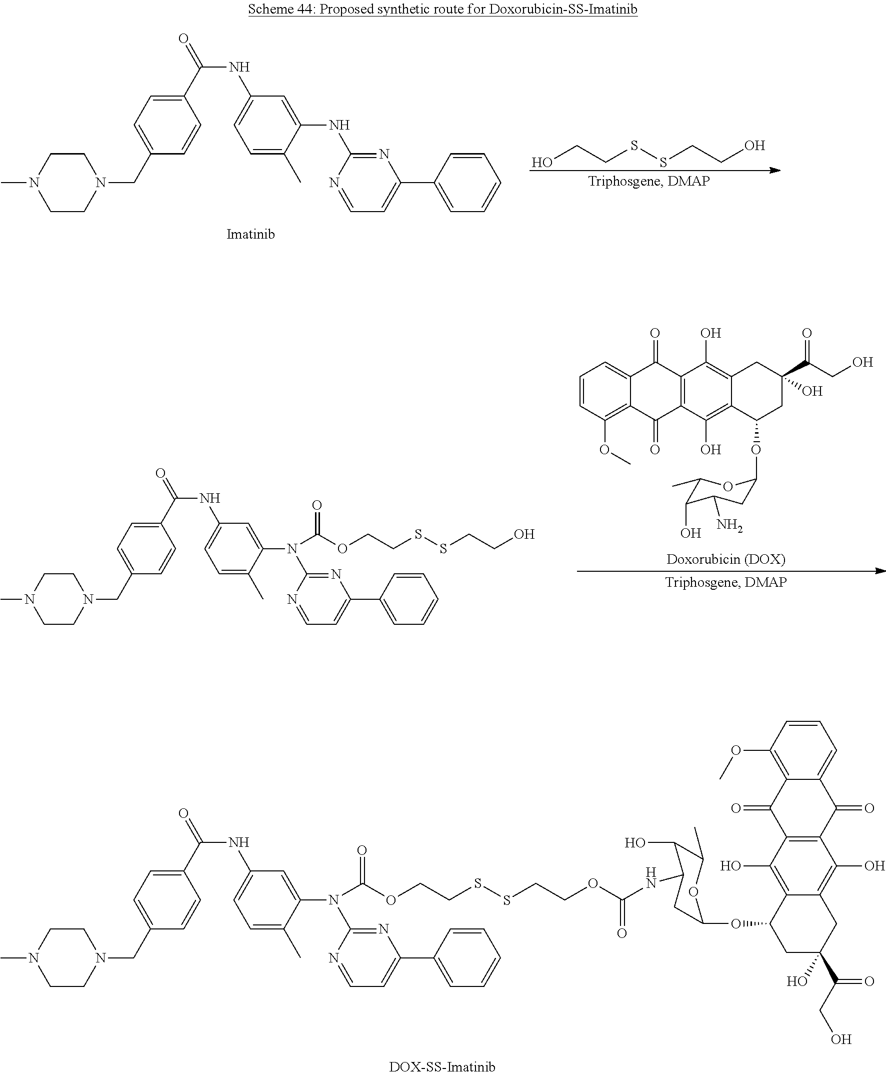 [SMILES: Cc1ccc(NC(=O)c2ccc(CN3CCN(C)CC3)cc2)cc1Nc1nccc(-c2ccccc2)n1 ; OCCSSCCO ; Cc1ccc(NC(=O)c2ccc(CN3CCN(C)CC3)cc2)cc1N(C(=O)OCCSSCCO)c1nccc(-c2ccccc2)n1 ; COc1cccc2c1C(=O)c1c(O)c3c(c(O)c1C2=O)C[C@@](O)(C(=O)CO)C[C@@H]3OC1CC(N)C(O)C(C)O1 ; COc1cccc2c1C(=O)c1c(O)c3c(c(O)c1C2=O)C[C@@](O)(C(=O)CO)C[C@@H]3OC1CC(NC(=O)OCCSSCCOC(=O)N(c2nccc(-c3ccccc3)n2)c2cc(NC(=O)c3ccc(CN4CCN(C)CC4)cc3)ccc2C)C(O)C(C)O1]

### Canertinib-Based DOX-Drug Conjugates

Provided below is the synthetic protocol for synthesizing DOX-drug conjugates featuring canertinib as the drug.

### Synthetic Scheme for the Synthesis of Doxorubicin-SS-Canertinib

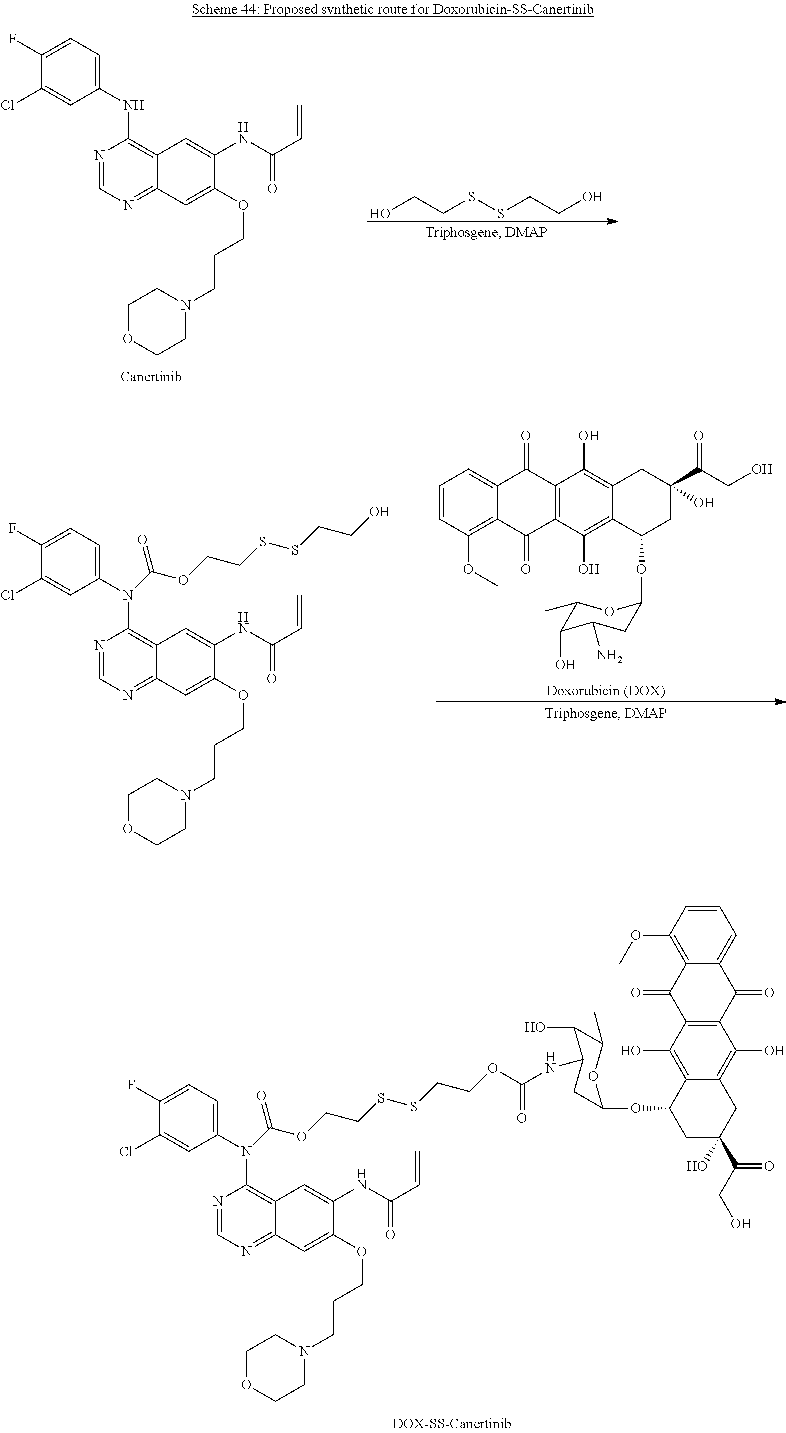 [SMILES: C=CC(=O)Nc1cc2c(Nc3ccc(F)c(Cl)c3)ncnc2cc1OCCCN1CCOCC1 ; OCCSSCCO ; C=CC(=O)Nc1cc2c(N(C(=O)OCCSSCCO)c3ccc(F)c(Cl)c3)ncnc2cc1OCCCN1CCOCC1 ; COc1cccc2c1C(=O)c1c(O)c3c(c(O)c1C2=O)C[C@@](O)(C(=O)CO)C[C@@H]3OC1CC(N)C(O)C(C)O1 ; C=CC(=O)Nc1cc2c(N(C(=O)OCCSSCCOC(=O)NC3CC(O[C@H]4C[C@](O)(C(=O)CO)Cc5c(O)c6c(c(O)c54)C(=O)c4c(OC)cccc4C6=O)OC(C)C3O)c3ccc(F)c(Cl)c3)ncnc2cc1OCCCN1CCOCC1]

### Ceritinib-Based DOX-Drug Conjugates

Provided below is the synthetic protocol for synthesizing DOX-drug conjugates featuring ceritinib as the drug.

### Synthetic Scheme for the Synthesis of Doxorubicin-SS-Ceritinib

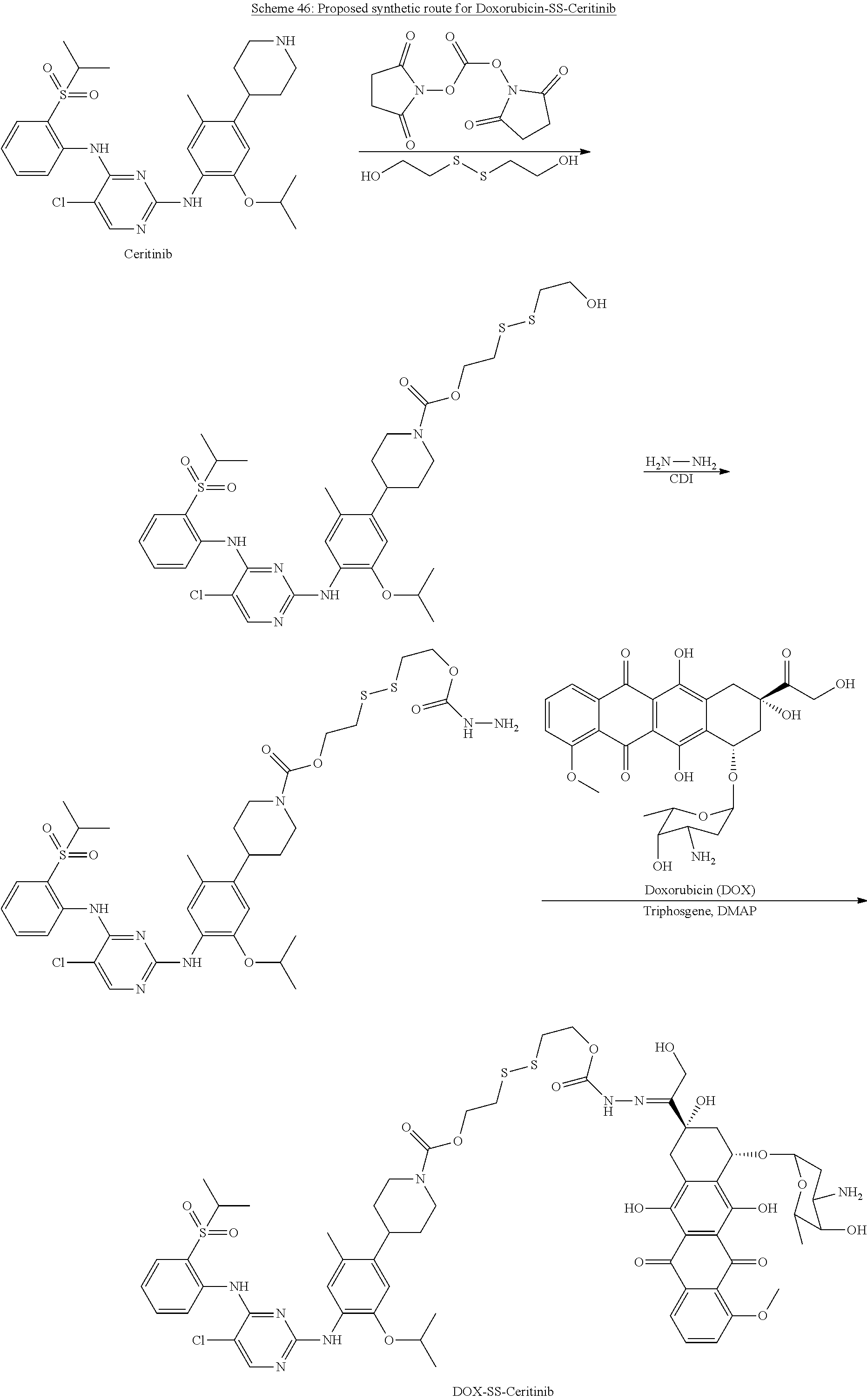

### Dabrafenib-Based DOX-Drug Conjugates

Provided below is the synthetic protocol for synthesizing DOX-drug conjugates featuring dabrafenib as the drug.

### Synthetic Scheme for the Synthesis of Doxorubicin-SS-Dabrafenib

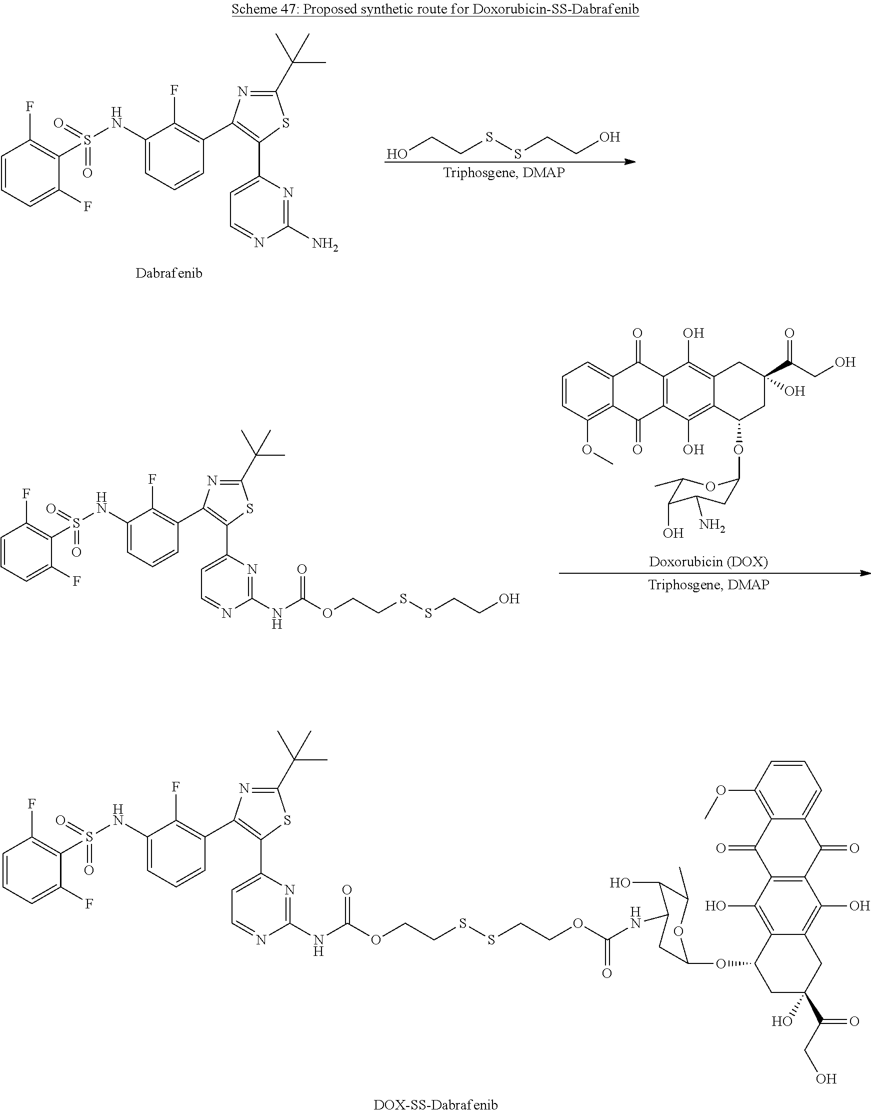 [SMILES: CC(C)(C)c1nc(-c2cccc(NS(=O)(=O)c3c(F)cccc3F)c2F)c(-c2ccnc(N)n2)s1 ; OCCSSCCO ; CC(C)(C)c1nc(-c2cccc(NS(=O)(=O)c3c(F)cccc3F)c2F)c(-c2ccnc(NC(=O)OCCSSCCO)n2)s1 ; COc1cccc2c1C(=O)c1c(O)c3c(c(O)c1C2=O)C[C@@](O)(C(=O)CO)C[C@@H]3OC1CC(N)C(O)C(C)O1 ; COc1cccc2c1C(=O)c1c(O)c3c(c(O)c1C2=O)C[C@@](O)(C(=O)CO)C[C@@H]3OC1CC(NC(=O)OCCSSCCOC(=O)Nc2nccc(-c3sc(C(C)(C)C)nc3-c3cccc(NS(=O)(=O)c4c(F)cccc4F)c3F)n2)C(O)C(C)O1]

### Vemurafenib-Based DOX-Drug Conjugates

Provided below is the synthetic protocol for synthesizing DOX-drug conjugates featuring vemurafenib as the drug.

### Synthetic Scheme for the Synthesis of Doxorubicin-SS-Vemurafenib

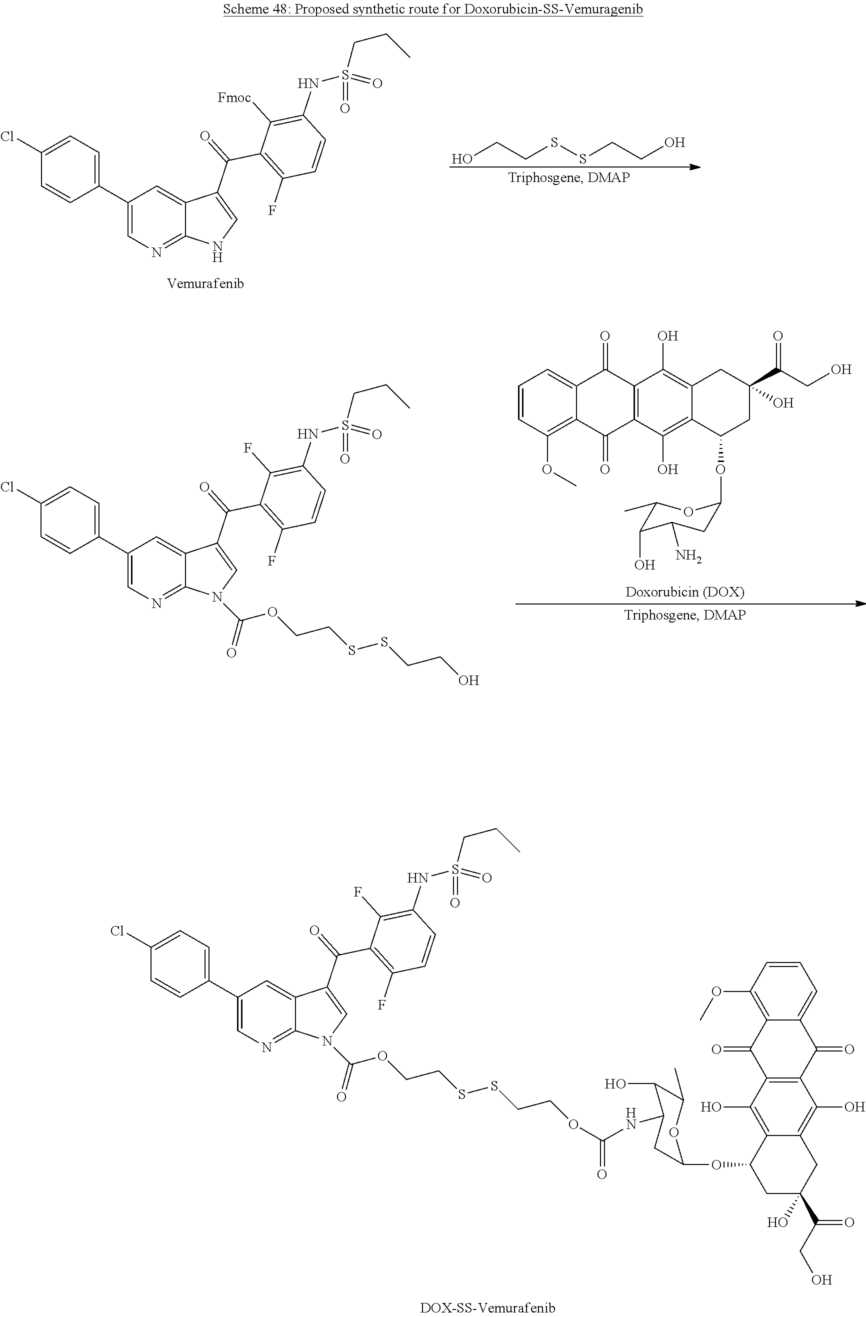

### Vorinostat-Based DOX-Drug Conjugates

Provided below is the synthetic protocol for synthesizing DOX-drug conjugates featuring vorinostat as the drug.

### Synthetic Scheme for the Synthesis of Doxorubicin-SS-Vorinostat

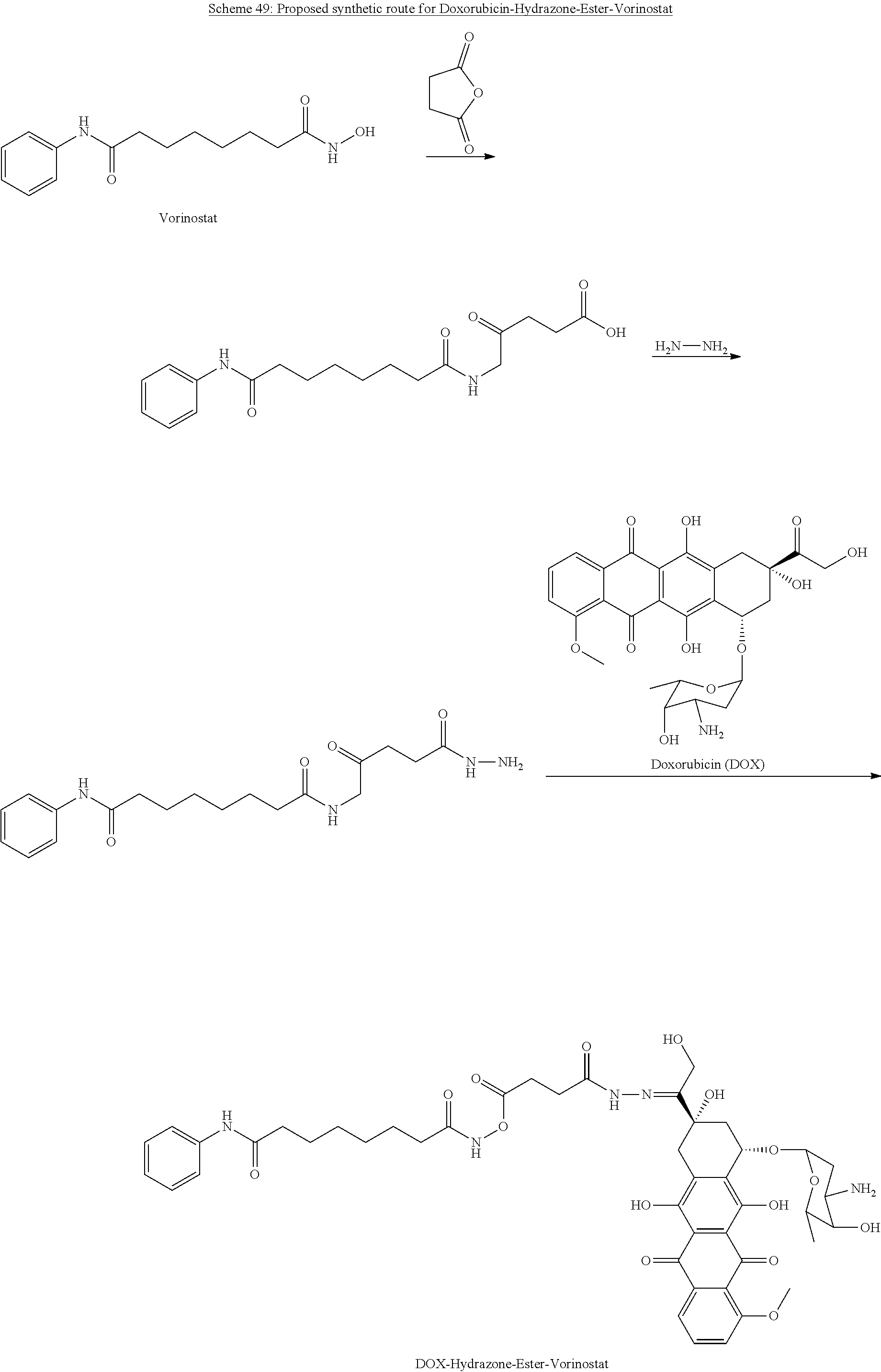

### Oxaliplatin-Based DOX-Drug Conjugates

Provided below is the synthetic protocol for synthesizing DOX-drug conjugates featuring oxaliplatin as the drug.

### Synthetic Scheme for the Synthesis of Doxorubicin-SS-Oxaliplatin

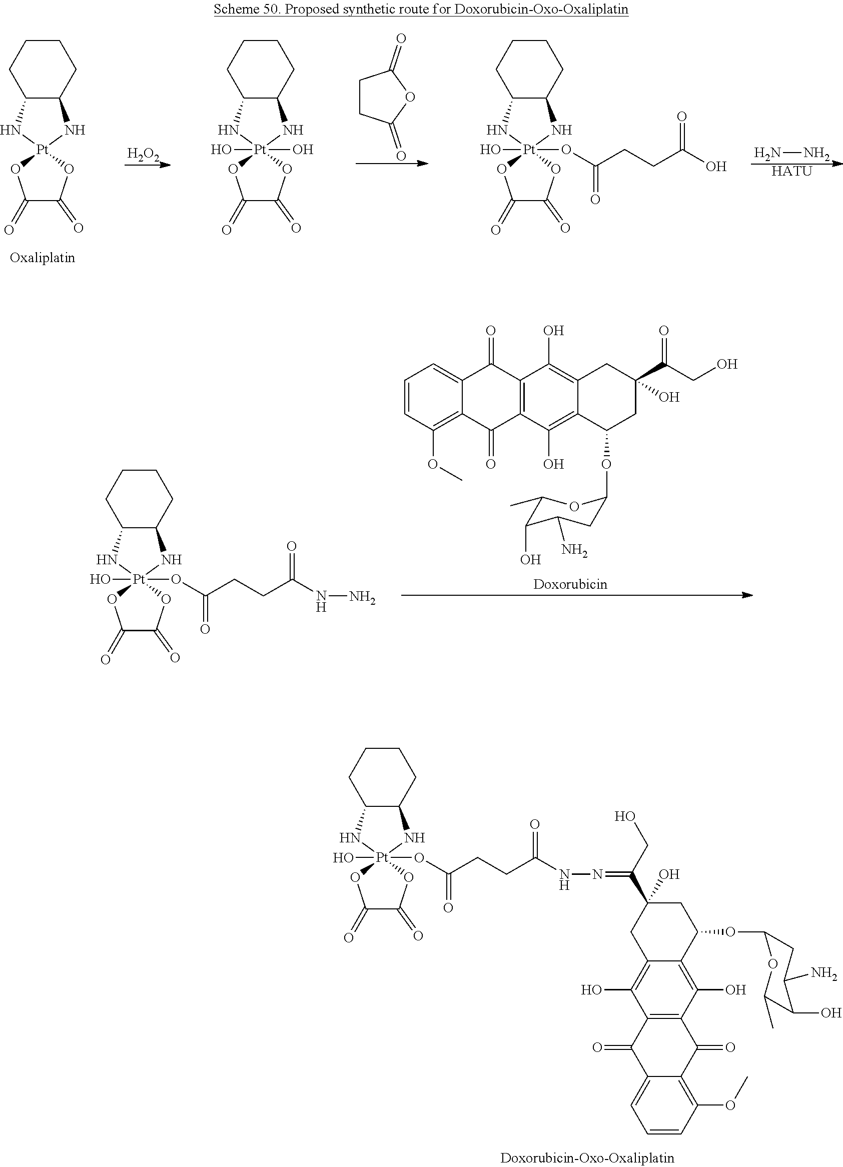

### Preladenant-Based DOX-Drug Conjugates

Provided below is the synthetic protocol for synthesizing DOX-drug conjugates featuring preladenant as the drug.

### Synthetic Scheme for the Synthesis of Doxorubicin-SS-Preladenant

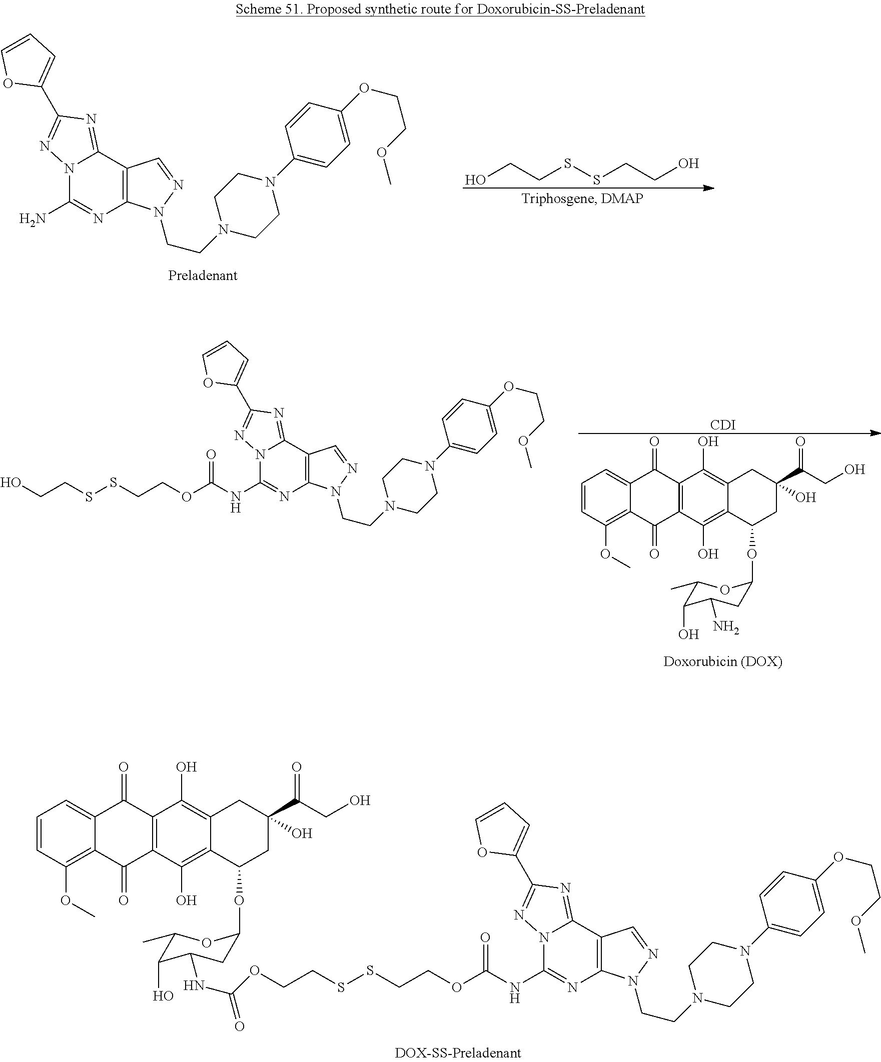

### Vipadenant-Based DOX-Drug Conjugates

Provided below is the synthetic protocol for synthesizing DOX-drug conjugates featuring vipadenant as the drug.

### Synthetic Scheme for the Synthesis of Doxorubicin-SS-Vipadenant

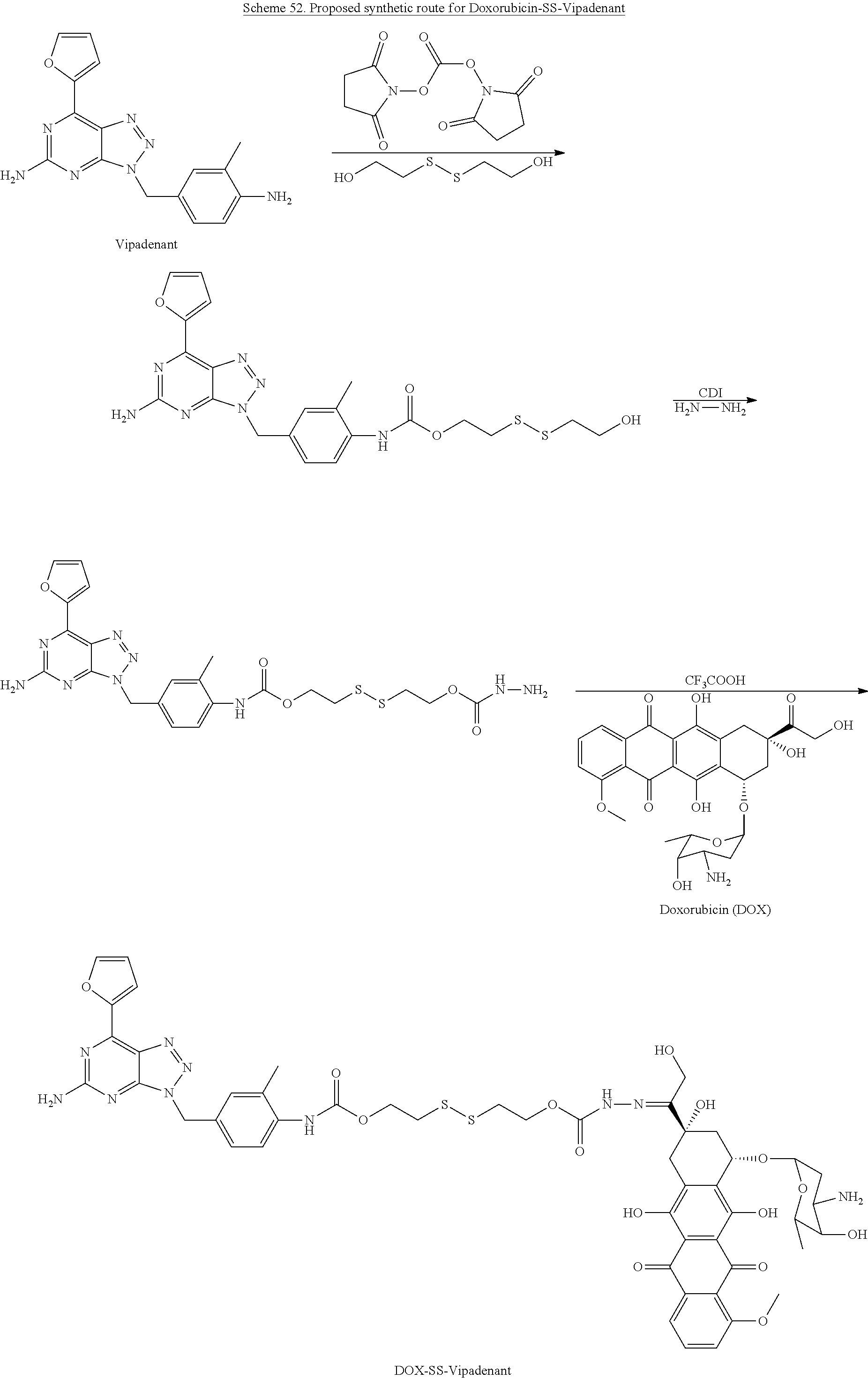

### ADU-S100-Based DOX-Drug Conjugates

Provided below is the synthetic protocol for synthesizing DOX-drug conjugates featuring ADU-S100 as the drug.

### Synthetic Scheme for the Synthesis of Doxorubicin-SS-ADU-S100

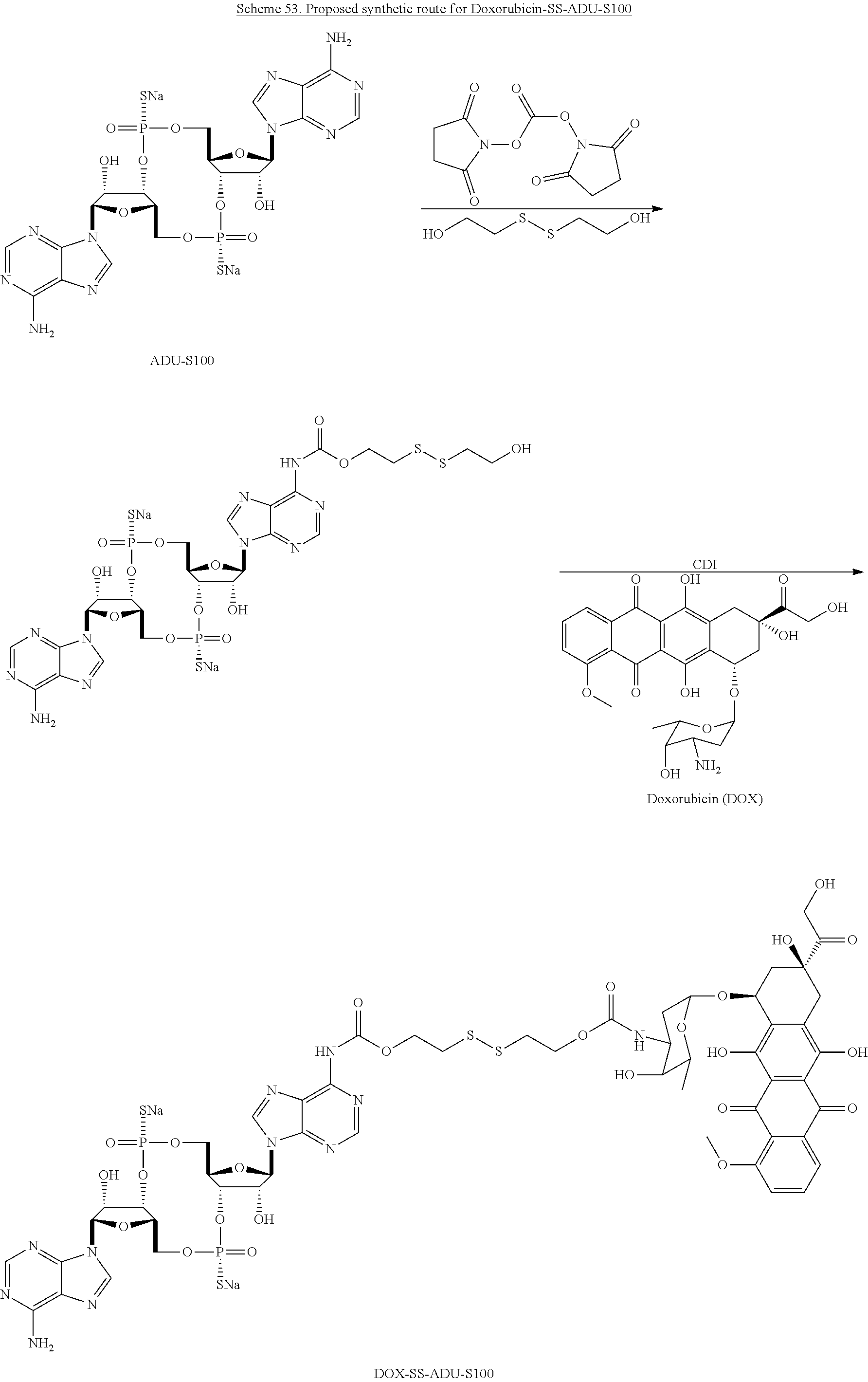

### Vadimezan-Based DOX-Drug Conjugates

Provided below is the synthetic protocol for synthesizing DOX-drug conjugates featuring vadimezan as the drug.

### Synthetic Scheme for the Synthesis of Doxorubicin-SS-Vadimezan

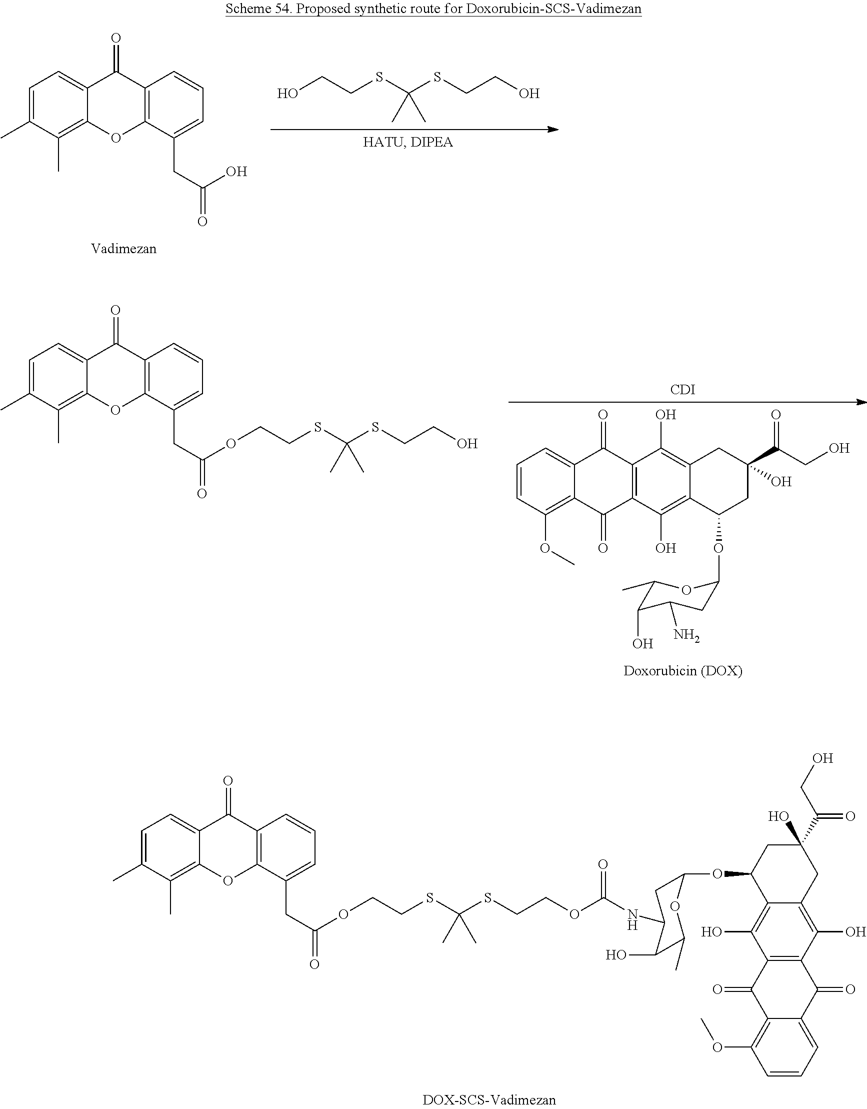

### Example 3—Nanovesicles

The following example provides the experimental details for preparing nanovesicles of the present disclosure as well as associated pharmacokinetics and biodistribution Studies and therapeutic efficacy investigations of the same.

### Experimental Details

### A. Cryogenic Transmission Electron Microscopy (Cryo-EM)

Liposomal suspensions (2 mg CPT/mL for Camptothesomes; 2.5 mg DOX-IND/mL for co-delivery Camptothesome-4, ˜7% DOX-IND DLC) were prepared for imaging by applying 3 microliters to the surface of a C-Flat 1.2/1.3 engineered TEM grid (Protochips, Morrisville, NC) immediately followed by either a 3 or 6 second blot at 100% RH in a FEI Vitrobot (Hillsboro, OR) prior to rapid emersion into liquid nitrogen cooled liquid ethane. Grids were transferred into a Phillips TF20 (Eindhoven, NL) operating at 120 KeV with a Gatan CT3500 side entry cryoholder (Pleasantville, CA) maintained at −180° C. Images were recorded on a TVIPS XF416 CMOS camera and measurements were performed within the EMMenu software package provided by TVIPS (Gauting, DE) for the operation of the XF416 camera.

### B. Immunohistochemistry

The tumor blocks from respective therapeutic efficacy studies were collected from sacrificed mice, fixed in 4% paraformaldehyde overnight, processed and then embedded by paraffin. Tumor blocks were cut into sections of 4 μm thickness, which were mounted on positively charged glass slides by the University of Arizona Cancer Center TACMASR Core facility for a series of IHC staining processes and procedures following established and standardized protocols. Briefly, the slides were loaded onto the Leica Bond RXm Autostainer with covertiles to prevent dehydration between staining steps. Slides were heated to 60° C. then deparaffinized. Slides were incubated in 10 mM tris-EDTA (pH 9) or 1 mM sodium citrate (pH 6) at 98° C. (85° C. for calreticulin; 50° C. for perforin) for epitope retrieval. After cooling down to ambient temperature, the slides were rinsed in TBS wash buffer and were subsequently treated with 3% H₂O₂ for 5 min to block endogenous peroxidase activity, and then incubated with individual primary antibodies for 15 to 50 minutes. Afterwards, the slides were rinsed with wash buffer and followed by incubation with HRP-conjugated anti-rabbit polymer; for Foxp3 and IL-10, rabbit anti-rat secondary antibody was used prior to incubation with HRP-conjugated anti-rabbit polymer at ambient temperature for 8 min. The slides were incubated with DAB (3,3′-Diaminobenzidine) for 10 minutes for visualization after being rinsed with wash buffer. The slides were then washed in distilled water, counterstained with Hematoxylin at room temperature for 5 minutes. The reagents were part of the Bond Polymer Refine Detection Kit (DS9800, Leica). Slides were unloaded from an autostainer, dehydrated in increasing concentrations of ethanol, three changes of xylene, mounted with media and cover-slipped. After staining, the slide sections were dried and observed under microscope (Nikon, Eclipse 50i, Japan) equipped with a digital camera. The slides were read by an experienced veterinary pathologist and the IHC staining quantitative analysis was performed by using the ImageJ Fiji software following the established protocol [56]. The quantification of the IHC staining intensity of each immune biomarker was obtained by dividing the mean DAB staining intensity value by the total number of nuclei measured in an image (6 fields/tumor×5 or 6 tumors/treatment). Afterwards, the respective IHC staining quantitative data was normalized to vehicle control samples.

### C. Antibodies Utilized for IHC Staining

Anti-interferon gamma (ab9657, 1/200), anti-PD-1 (ab137132, 1/500), anti-IDO (ab106134, 1/300), anti-HMGB1 (ab18256, 1/400), anti-TLR4 (ab13867, 1/100), anti-IL-12 (ab131039, 1/500), anti-IL-10 (ab189392, 1/100), anti-LRP1 (ab92544, 1/750), anti-CD8α (ab209775, 1/100), anti-perforin (ab16074, 1/600), anti-granzyme B (ab4059, 1/100), and anti-calreticulin (ab2907, 1/400) were obtained from Abcam; Anti-PD-L1 (#13684T, 1/75) and anti-cleaved caspase-3 (#9664S, 1/300) were purchased from Cell Signaling; Anti-Foxp3 (#13-5773-82, 1/100) was from Invitrogen. All antibodies were diluted in Bond Primary Antibody Diluent (AR9352, Leica).

Foxp3 and IL-10 used a rabbit anti-mouse secondary antibody prior to incubating with the HRP-conjugated anti-rabbit polymer. TLR4, LRP1, HMGB1, Granzyme B, CD8, CC3, IL-12, PD-L1, and Calreticulin did not require a secondary antibody as they were rabbit antibodies and the HRP-conjugated polymer on the staining kit is against rabbit.

### Self-Assembly of SM-Derived CPT Conjugates

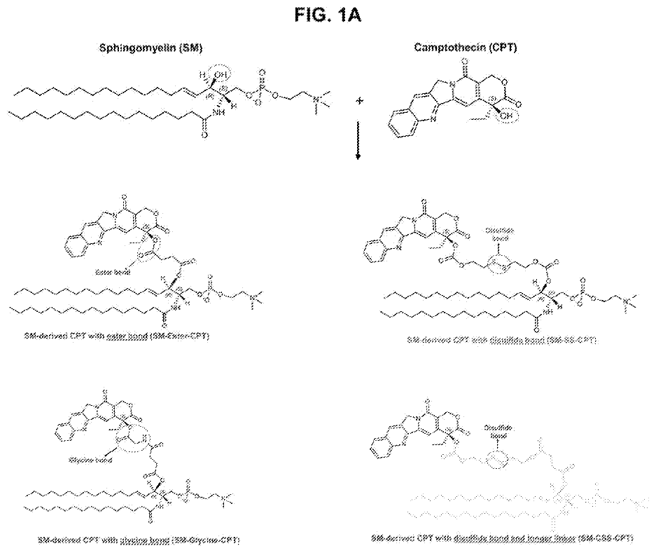

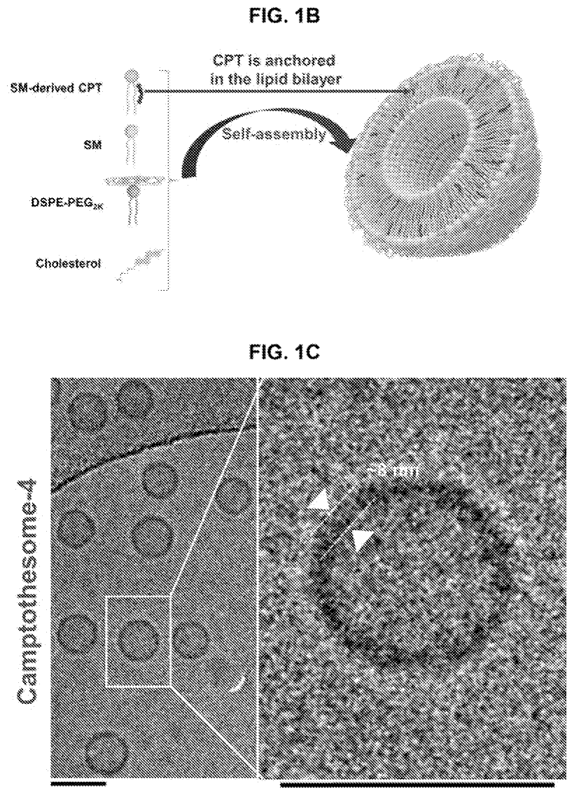

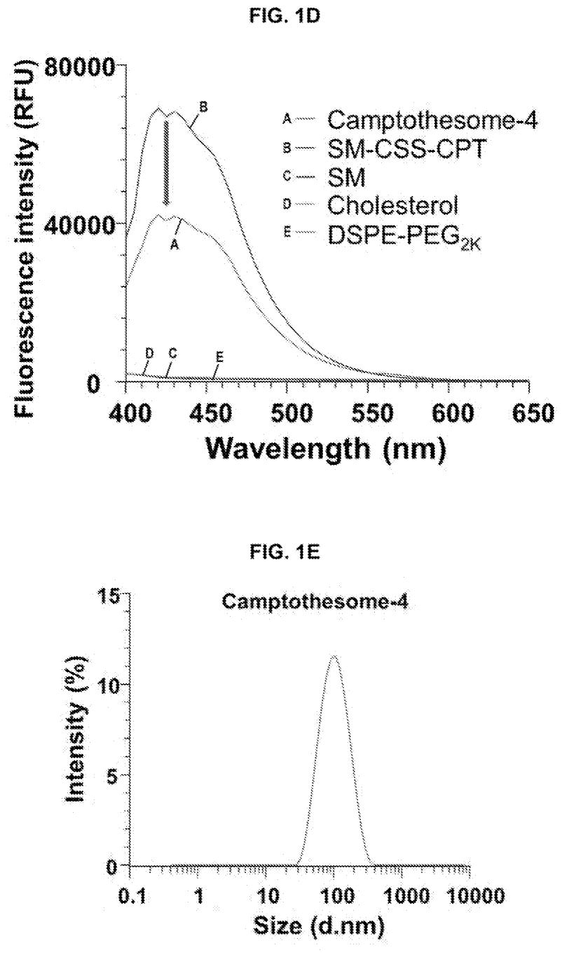

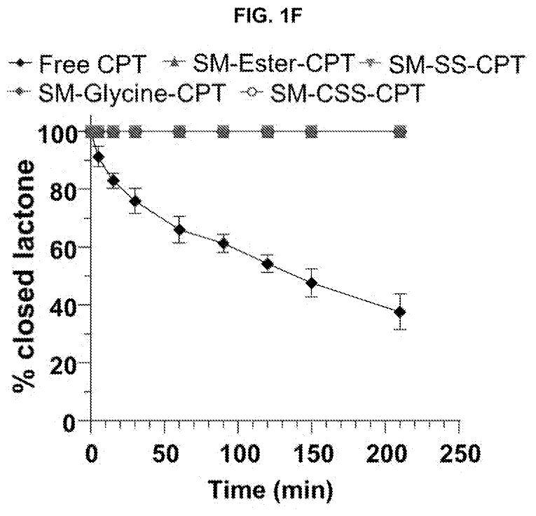

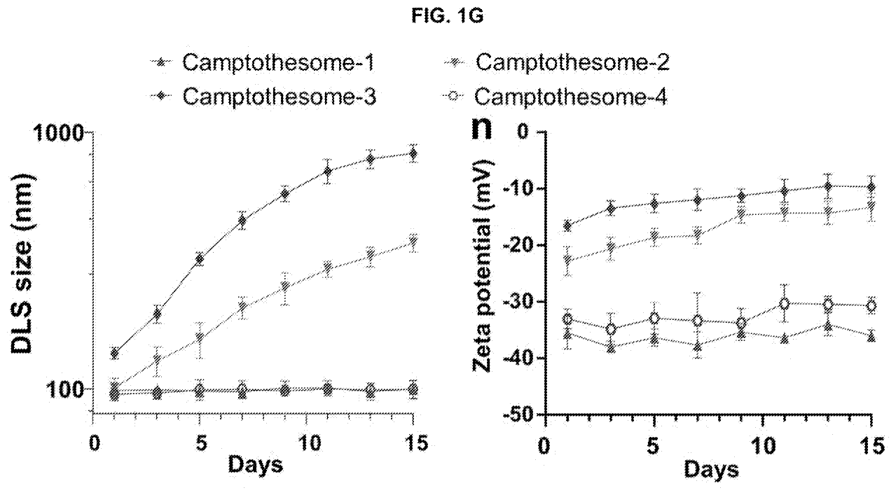

The self-assembly of SM-derived CPT conjugates into liposomal nanovesicles was prepared by standard thin-film hydration method [43,44]. Briefly, an appropriate ratio of SM, cholesterol, and DSPE-PEG2K (Avanti Polar Lipids) and SM-conjugated CPT (SM-Ester-CPT, SM-SS-CPT, SM-Glycine-CPT, or SM-CSS-CPT) as listed in FIG. 1B were dissolved in ethanol in a 100 mL round bottom glass flask. The solvent was evaporated under a rotatory evaporator (RV 10 digital, IKA®) to generate a thin film, which was further dried under ultra-high vacuum (MaximaDry, Fisherbrand) for 0.5 h. The film was hydrated with a 5% dextrose aqueous solution at 50° C. for 30 min, and then sonicated for 12 min by using a pulse 3/2 s on/off at a power output of 60 W. To remove any unencapsulated SM-CPT, the nanoparticles underwent ultra-centrifugation at 100,000×g for 45 min. Dynamic light scattering (DLS) size and zeta potential, morphology, and CPT content of the purified Camptothesome nanovesicles were determined by the Zetasizer Nano (Nano-ZS, Malvern Panalytical), cryogenic transmission electron microscopy (cryo-EM), and HPLC, respectively. CPT drug loading capacity (DLC, Equation 1) was calculated as below:

$$\frac{weight of conjugated CPT in Camptothesome}{weight of Camptothesome} \times 100 \% \qquad Equation 1$$

### Preparation of DOX-IND/Camptothesome-4 and Folate/DOX-IND/Camptothesome-4 Nanoformulations

For remotely loading DOX-derived drug(s) into above prepared nanovesicles, an appropriate ratio of SM, SM-CSS-CPT, cholesterol, and DSPE-PEG2K (addition of 0.5 molar % of DSPE-PEG2K-Folate (Avanti Polar Lipids) for Folate/DOX-IND/Camptothesome-4) were dissolved in ethanol with a 100 mL round bottom glass flask. The solvent was evaporated under a rotatory evaporator to generate a thin film, which was further dried under ultra-high vacuum for 0.5 h. The film was hydrated with an 80 mM (NH₄)₂SO₄ aqueous solution at 50° C. for 30 min, and then sonicated for 12 min by using a pulse 3/2 son/off at a power output of 60 W. The free (NH₄)₂SO₄ was removed by a PD-10 column (Sephadex G-25, GE Healthcare) using PBS as eluent. The remote DOX-IND loading was achieved by incubated (NH₄)₂SO₄/Camptothesome-4 with 2-10 mg/mL DOX-IND at 65° C. for 1 h. After cooling down in 4° C. for 30 min, the free DOX-IND was removed by running through a PD-10 column. The size and zeta potential, morphology, and drug content of the DOX-IN D/Camptothesome-4 and Folate/DOX-IND/Camptothesome-4 nanovesicles were determined by DLS, cryo-EM and HPLC, respectively. The DOX-IND DLC (Equation 2) and drug loading efficiency DLE, (Equation 3) were calculated using the formulas shown below:

$$\frac{weight of encapsulated drug}{weight of ( Camptothesome + encapsulated drug )} \times 100 \% \qquad Equation 2$$

$$\frac{weight of encapsulated drug}{weight of input drug )} \times 100 \% \qquad Equation 3$$

### Fluorescence Quenching Assay

Camptothesomes and DOX-IND/Camptothesome-4 were prepared as described above. Controls include respective SM-CPT conjugates, SM, Cholesterol, DSPE-PEG2K, and/or DOX-IND. Various samples at eq. 100 μM in 200 μL were placed into a 96-well plate (Greiner Bio-One UV-Star™), and the fluorescence intensity was detected on a SpectraMax M3 reader (Molecular Devices, San Jose, CA), employing an excitation wavelength of 360 nm and emission wavelength from 400 to 650 nm for CPT and SM-CPT conjugates, and an excitation wavelength of 470 nm and emission wavelength from 520 to 700 nm for DOX-IND.

### Maximum Tolerated Dose (MTD)

Groups of 3 BALB/c m ice were administered intravenously (IV) with free CPT (5, 7.5, 10, and 12.5 mg CPT/kg, formulated in 10% Tween 80/0.9% NaCl (9:1, v/v) with 20 min sonication by the probe) [47], Camptothesome-1 (50, 65, 80, 100, and 120 mg CPT/kg), Camptothesome-2 (15, 25, 35, 40, and 45 mg CPT/kg), Camptothesome-3 (15, 25, 30, 35, 40, 45, 60, and 80 mg CPT/kg), Camptothesome-4 (15, 25, 30, 35, 40, and 45 mg CPT/kg) and DOX-IND/Camptothesome-4 (5/15, 6.7/20, 8.3/25, and 10/30 mg DOX-IND/CPT mg/kg), 5% dextrose served as the vehicle control. Changes in body weight and survival of mice were followed every 1-2 days for two weeks. The MTD was defined as the dose that causes neither mouse death due to the toxicity nor greater than 15% of body weight loss or other remarkable changes in the general appearance within the entire period of the experiments. On day 14 post drug injection, blood was withdrawn by cardiac puncture and major organs (e.g., heart, liver, and kidneys) were collected. Blood was collected in lithium heparin tubes (BD Microtainer™) followed by centrifuging at 2,000×g for 10 minutes in a refrigerated centrifuge. The supernatant (serum) was sent to University of Arizona University Animal Care Pathology Services Core for a series of serum chemistry analysis. The whole blood in dipotassium EDTA tube (BD Microtainer™) were used for leukocytes, erythrocytes, and thrombocytes analysis. Mice organs from MTD dose and vehicle control groups were placed in a 4% paraformaldehyde solution for 24 h and then sent to Tissue Acquisition and Cellular/Molecular Analysis Shared Resource (TACMASR) at University of Arizona Cancer Center for histopathological analysis.

### Cells and Mice

CT26 and CT26-Luc were obtained from University of Arizona Cancer Center and cultured in complete RPMI-1640 medium. MC38 was purchased from Kerafast and cultured in complete DMEM medium. B16-F10-Luc2 was obtained from ATCC and cultured in complete DMEM medium. All the cell lines were cultured in the corresponding medium containing 10% FBS, 100 U/mL penicillin, 100 μg/mL streptomycin, and 2 mM L-glutamine at 37° C. in a CO₂ incubator. BALB/c and C57BU6 mice (Charles Rivers, 6-8 weeks old, male and female) were used. Tumor size was measured by a digital caliper at indicated times and calculated according to the formula=0.5×length×width². Mice were euthanized and removed from the respective study when individual tumor reached ˜2000 mm³ in size or animals became moribund with severe weight loss. The animals were maintained under pathogen-free conditions and all animal experiments were approved by the University of Arizona Institutional Animal Care and Use Committee (IACUC).

### Pharmacokinetics and Biodistribution Studies

### A. In Subcutaneous (SC) CT26 Tumor Bearing Mice.

Following a single IV injection via tail vein of free CPT (5 mg CPT/kg, MTD) and different Camptothesomes (20 mg CPT/kg) to SC CT26 tumor bearing mice (n=3, ˜300 mm³), blood was withdrawn at 0.083, 0.333, 1, 2.5, 8, 12 and 24 h and plasma (via plasma tube, BD Microtainer™) was collected and digested in methanol (90 μL methanol/10 μL serum) for HPLC analysis to measure the released CPT and SM-derived CPT conjugates. 24 h after drug IV administration, tumor tissues and major organs (heart, liver, spleen, lung, and kidneys) were collected, weighed, and then homogenized in acidified methanol (0.075 M HCl, 900 μL/100 mg tissues) followed by drug content determination using an established HPLC method. To unravel the intratumoral CPT release rate in a shorter and longer time period for Camptothesome-4, a separate biodistribution experiment was performed, where SC CT26 bearing mice (n=3, ˜300 mm³) received a single IV injection of Camptothesome-4 (20 mg CPT/kg). Mice were sacrificed at 2.5 h and 72 h post drug administration, the drug content in the collected tumor tissues and organs were processed, and analyzed as described above.

### B. In Orthotopic CT26-Luc Tumor-Bearing Mice.

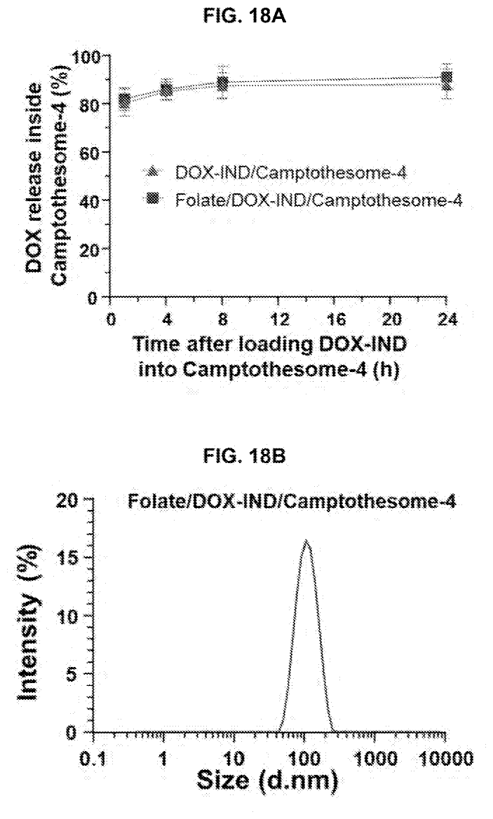

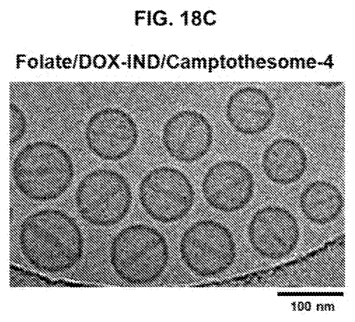

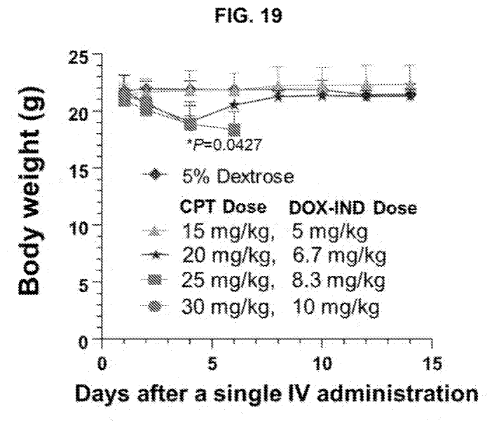

Various drug formulations [free CPT (5 mg/kg), IND (1.7 mg/kg), Doxil® (4.0 mg/kg), DOX-IND/Camptothesome-4 (6.7/20 mg DOX-IND/CPT/kg, 2% DOX-IND) and Folate/DOX-IND/Camptothesome-4 (6.7/20 mg DOX-IND/CPT/kg)] were IV injected to orthotopic CT26-Luc tumor bearing mice (n=3, ˜300 mm³) via tail vein based on the MTD (FIG. 18). At 0.083, 0.333, 1, 2.5, 8, 12, and 24 h post drug administration, blood was withdrawn, and plasma was digested in methanol prior to HPLC measurement for the DOX, IND, DOX-IND, CPT, and SM-CSS-CPT. At 24 h, tumor tissues and major organs were collected and homogenized in acidified methanol (0.075 M HCl, 900 μL/100 mg tissues) before HPLC drug content analysis (Table 2; 19). The various pharmacokinetics parameters were assessed by one-compartmental model using PKSolver software [48].

TABLE 2
Physicochemical characterizations of Cy5.5/Camptothesome-4 with regards to size,
zeta potential, and polydispersity using different molar ratios of DSPE-Cy5.5.

| | DSPE-Cy5.5 | Z-Average Size | Zeta potential | |
|---|---|---|---|---|
| Formulation | (w/w %) | (d · nm) | (mV) | Polydispersity |
| Cy5.5/ | 0.1% | 169.1 ± 10.51 | −28.9 ± 2.50 | 0.204 ± 0.047 |
| Camptothesome-4 | 0.2% | 95.5 ± 4.12 | −31.0 ± 3.28 | 0.167 ± 0.022 |
| | 0.3% | 118.3 ± 5.76 | −28.7 ± 1.00 | 0.210 ± 0.034 |

### C. Visualization of In Vivo Tumor Delivery and Deep Tumor Penetration.

To visualize Camptothesome-4 tumor delivery in vivo, Camptothesome-4 was labeled with 0.2% w/w DSPE-Cy5.5. SC CT26 tumor bearing mice (n=4, ˜300 mm³) were IV injected once with Cy5.5/Camptothesome-4 (20 m g CPT/kg). M ice were imaged at 0 h (prior to drug administration), and 2.5, 8, 12, and 24 h post IV injection (excitation=675 nm; emission=710 nm). At 24 h, tumors, heart, liver, spleen, lung, and kidneys were imaged using Lago optical imager. For tumor penetration investigation, tumors were frozen in an acetone/dry ice mixture prior to immunofluorescence examination. The tumor blood vessels were stained with a primary anti-CD31 (a.k.a. PECAM-1) antibody (Abcam, ab28364, 1:50), followed by an Alexa Fluor 488-conjugated secondary antibody (Abcam, ab150073, 1:400). DAPI was used to localize the cellular nuclei. Tumor tissues were cut into 5 mm slide section by University of Arizona TACMASR and subject to the confocal laser scanning microscopy using Leica SP5-II confocal microscope (Buffalo Grove, IL) at University of Arizona Cancer Center Imaging Core.

### Therapeutic Efficacy Investigation of Camptothesome Nanovesicles

### A. In SC CT26 Tumor Bearing Mice.

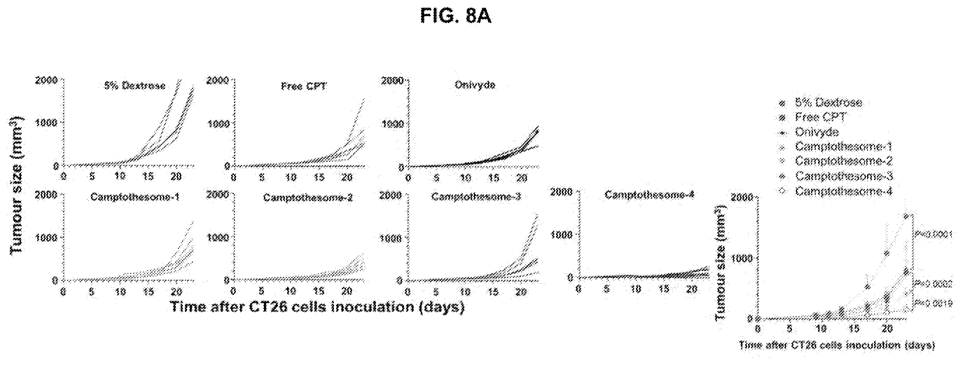

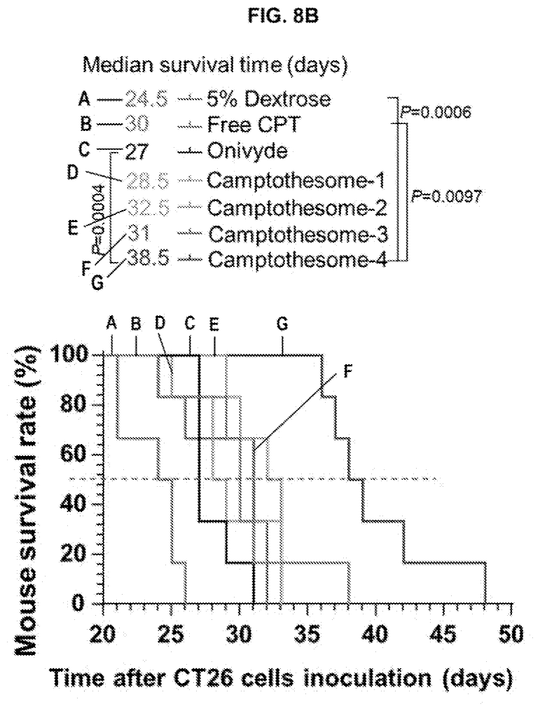

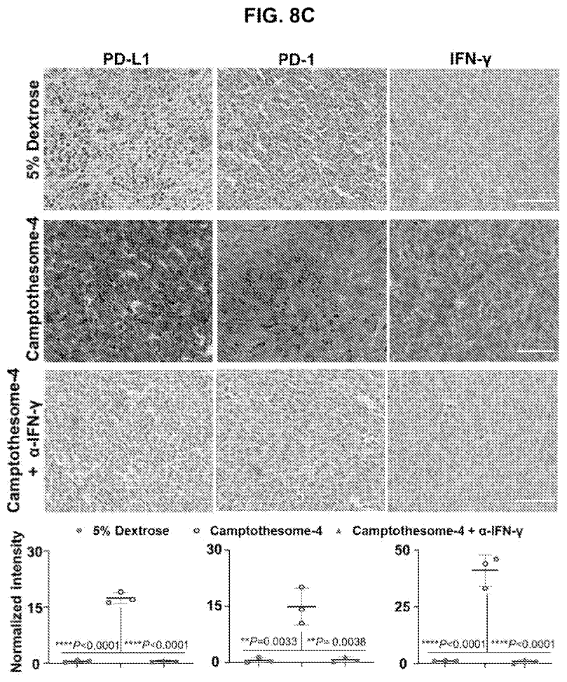

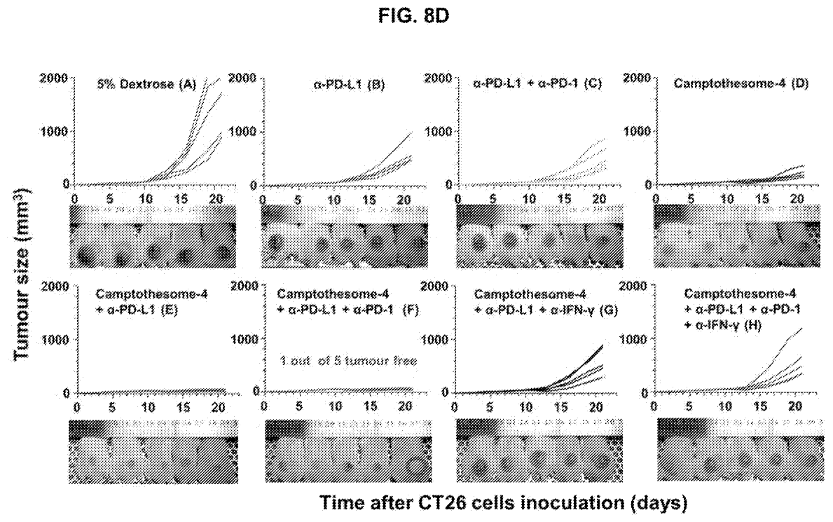

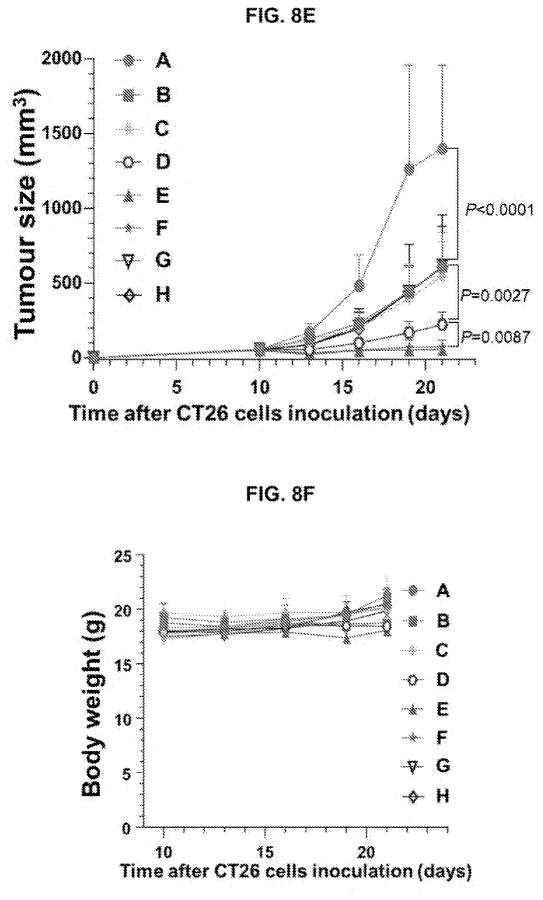

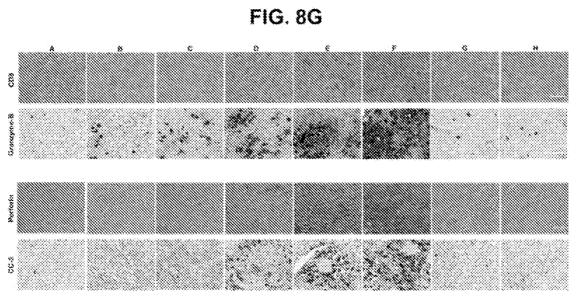

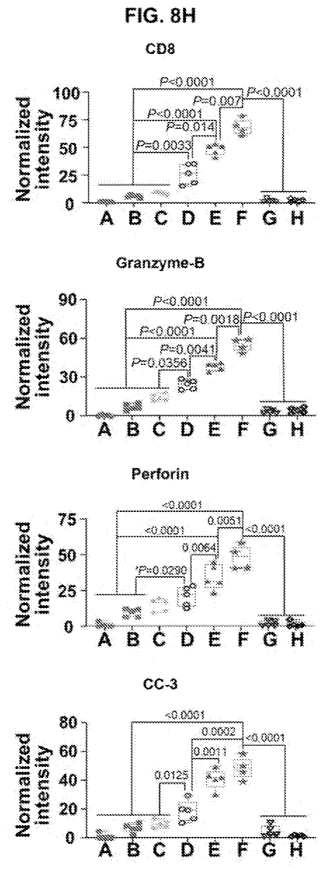

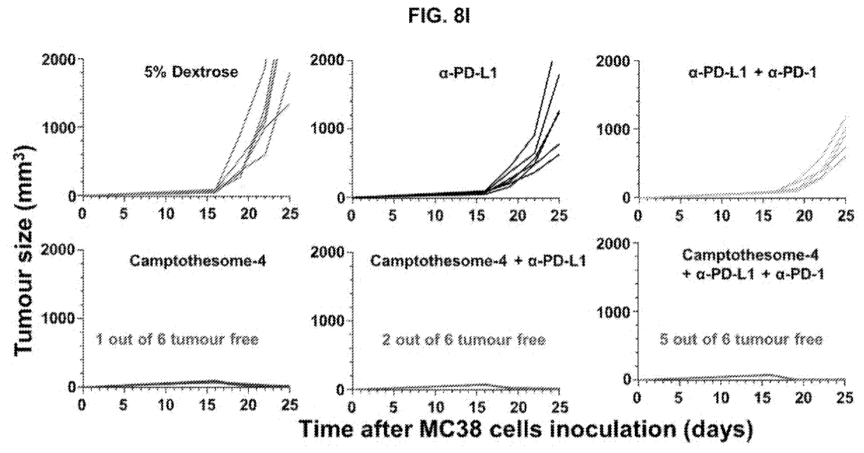

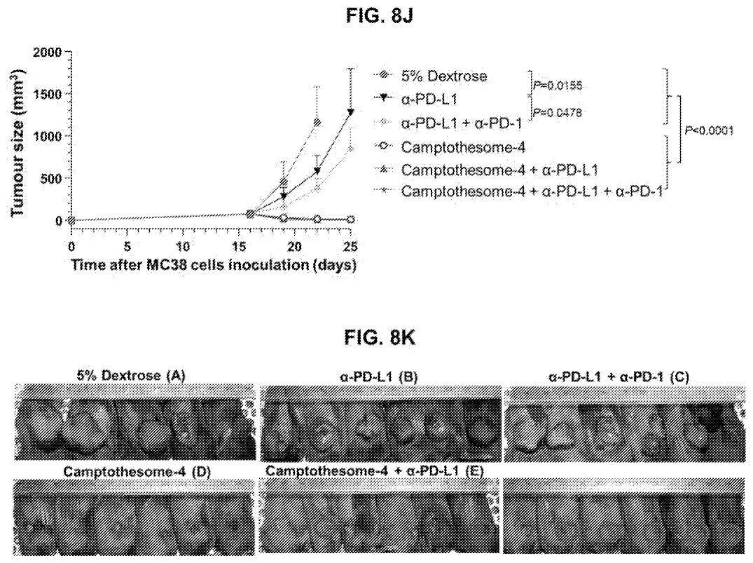

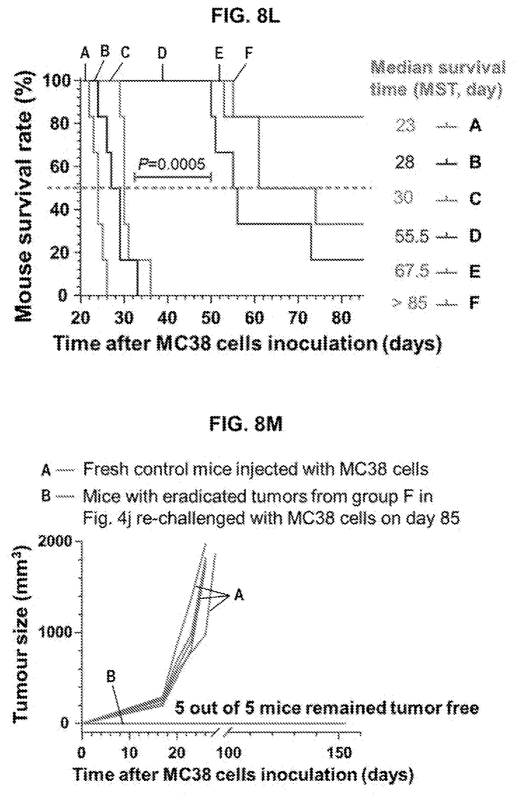

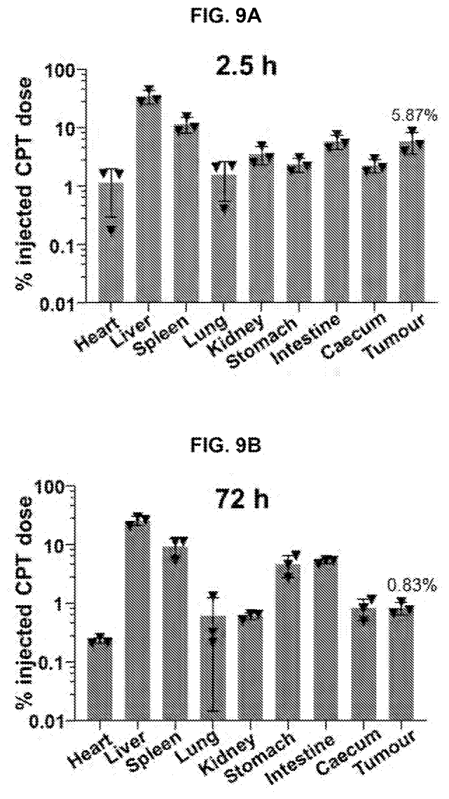

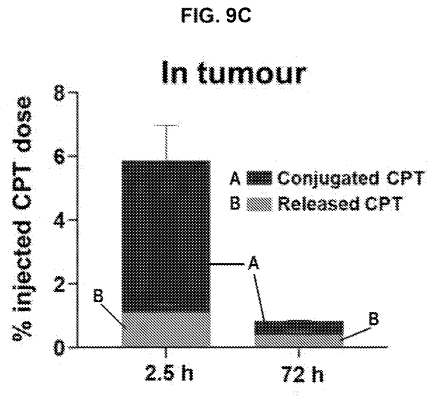

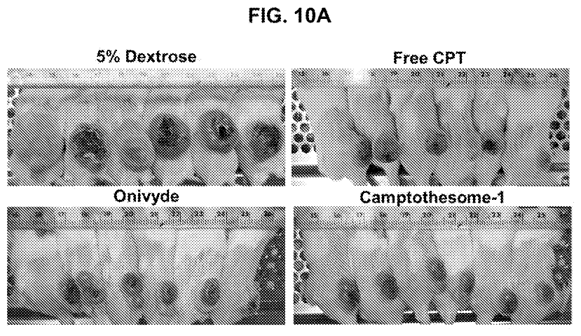

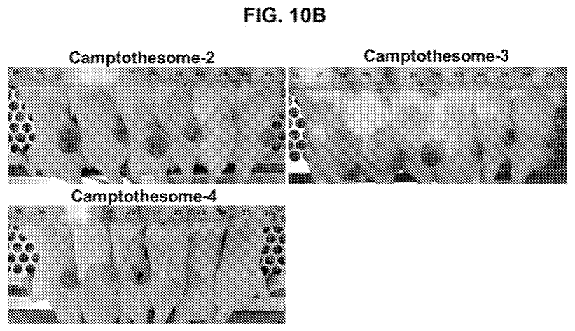

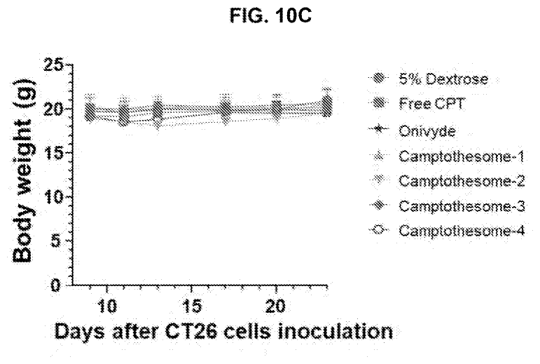

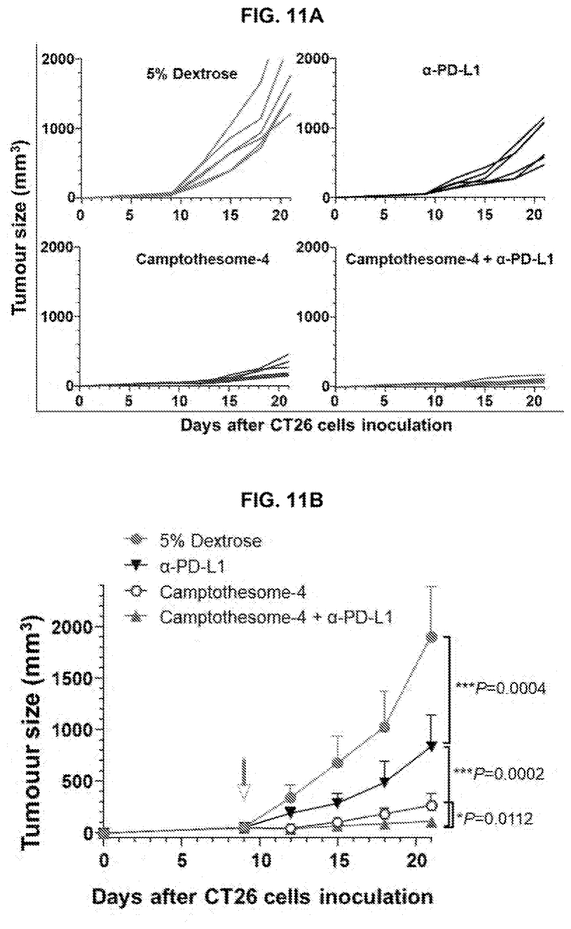

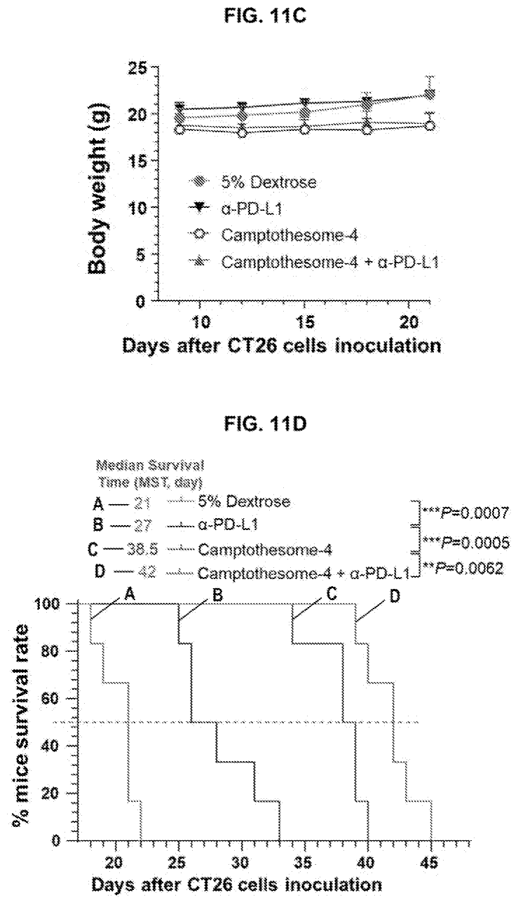

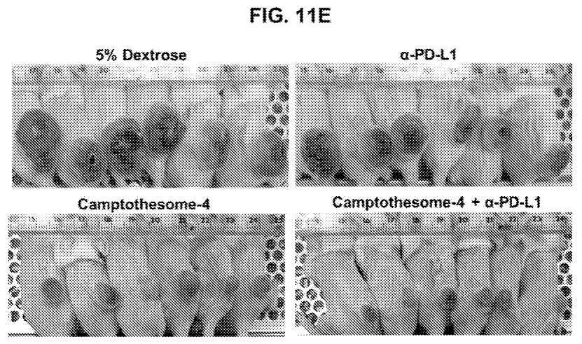

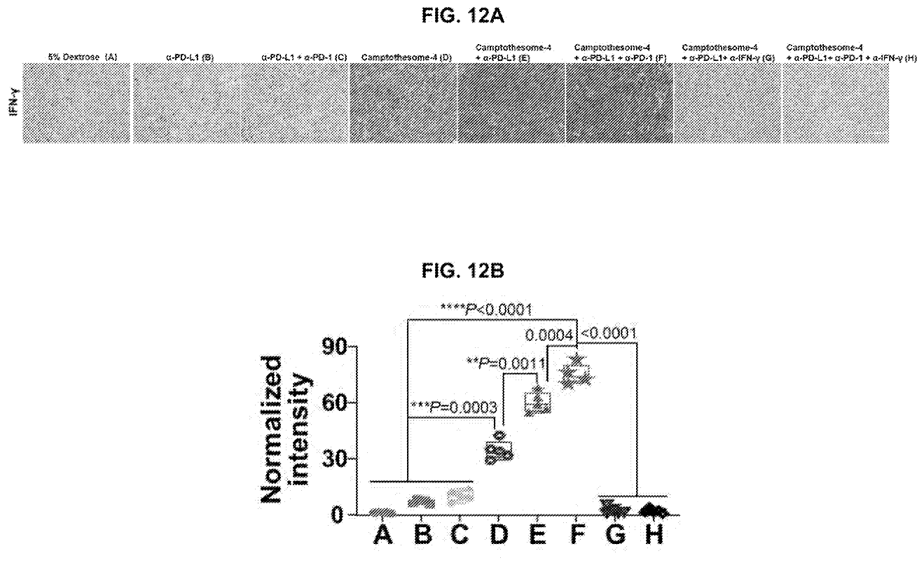

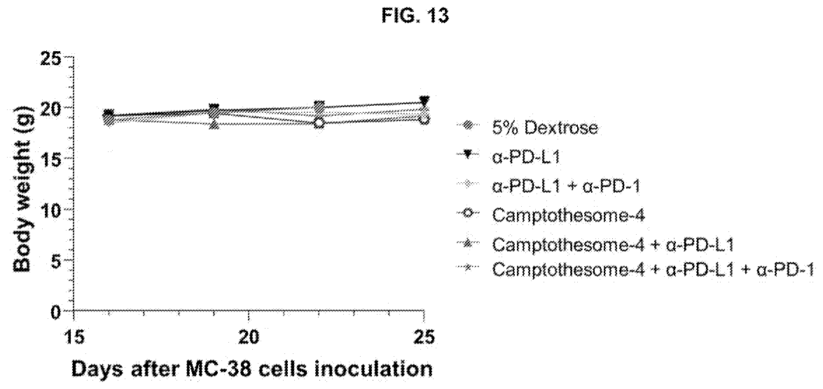

1×10⁵ CT26 cells in 100 μL of serum free medium were SC injected in the right flank of the BALB/c mice (n=5 or 6). When tumors grew to ˜50 mm³ in size, mice received one time IV administration of 5% dextrose (vehicle control), free CPT (5 mg CPT/kg), Onivyde® (20 mg irinotecan/kg) and different Camptothesomes (20 mg CPT/kg), or combination of Camptothesome-4 with IP injected α-PD-L1 (BioXCell, clone 10F.9G2, FIG. 11) with or without α-PD-1 (BioXCell, clone RMP1-14) at 100 μg/mouse/3 days for 3 times and with or without IP administered α-IFN-γ (BioXcell, clone R4-6A2, 200 μg/mouse/3 days). To determine the effect on survival rate in each group (n=6). Kaplan-Meier plots were used to express animal survival rate. For the efficacy study in FIG. 8D-8H, mice were euthanized on day 21; tumors were collected and soaked in 4% paraformaldehyde overnight prior to the immune phenotypic [CD8, Granzyme B, Perforin, Cleaved caspase-3 (CC-3), and IFN-γ] analysis using IHC staining by University of Arizona TACMASR.

In an independent study, CT26 tumor bearing mice (n=3, ˜200 mm³) received a single IV injection of Camptothesome-4 (20 mg CPT/kg) with or without α-IFN-γ as described above with 5% dextrose as vehicle control. 7 days later, IHC staining for PD-L1, PD-1, IFN-γ, and IDO in tumors were performed.

### B. In SC MC38 Tumor Bearing Mice.

In FIG. 8I-8J, 2×10⁵ cells/mouse in 100 μL of serum free medium were SC injected to C57BL/6 (n=6). When tumors reached ˜50 mm³, mice were IV injected once with Camptothesome-4 (20 mg CPT/kg) or in combination with α-PD-L1 or α-PD-L1+α-PD-1. α-PD-L1 and α-PD-1 were IP administered as mentioned above. Mice survival rate was closely monitored every day.

SC re-challenge the mice with eradicated tumors with MC38 cells to demonstrate memory T cell immunity. 5 tumor-free survivors in the Camptothesome-4 plus α-PD-L1+α-PD-1 group and 5 fresh healthy C57BL/6 mice (control mice) were SC injected MC38 cells (2×10⁵ cells/mouse) in the contralateral flank on day 85. The 5 control mice developed tumors uncontrollably, while the 5 surviving mice from Camptothesome-4 plus α-PD-L1+α-PD-1 group remained tumor free (FIG. 8M). Mouse weight was monitored every 3 days, and mice survival was monitored every day.

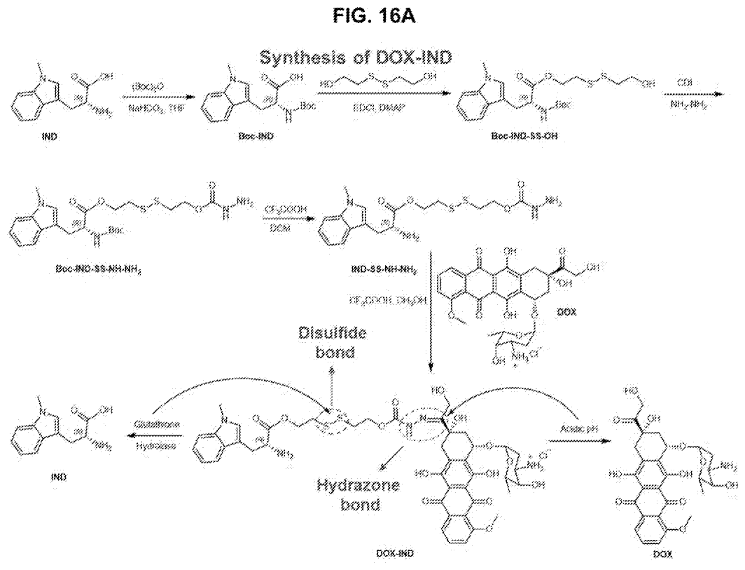

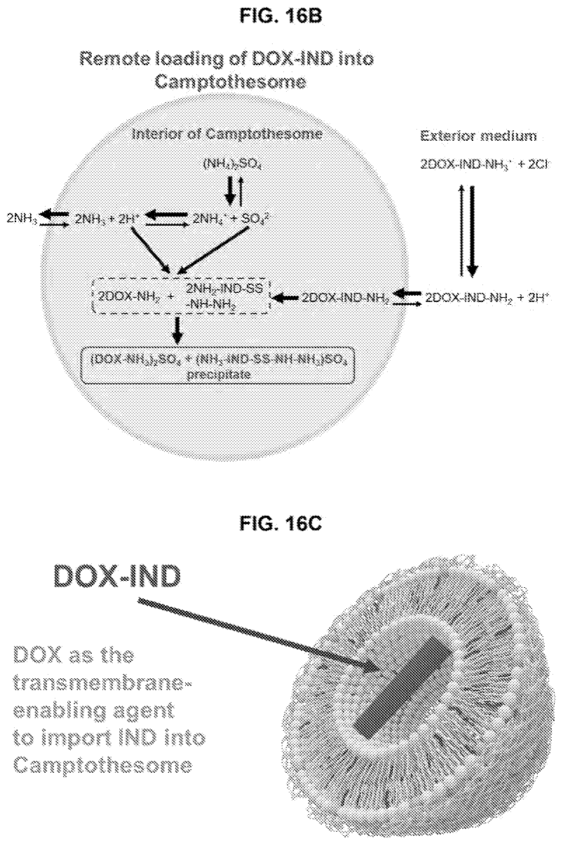

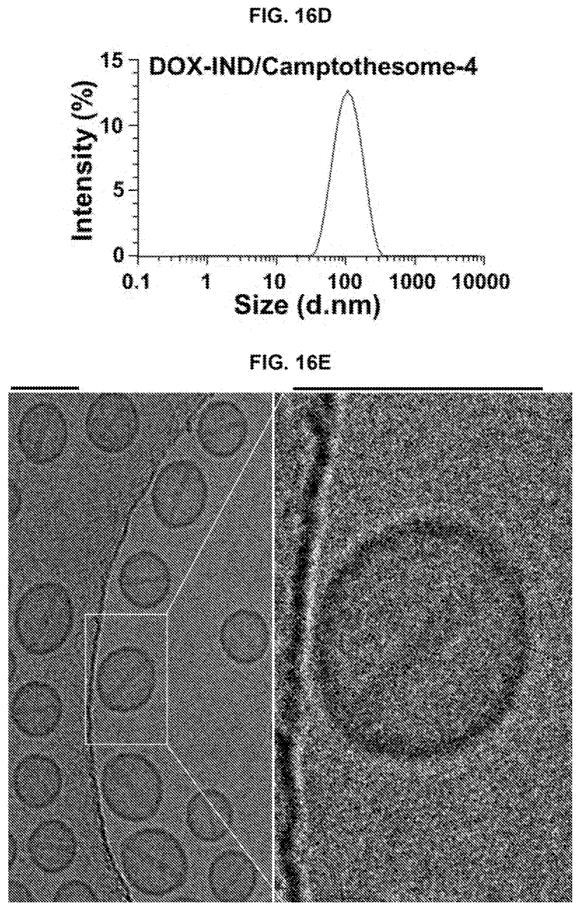

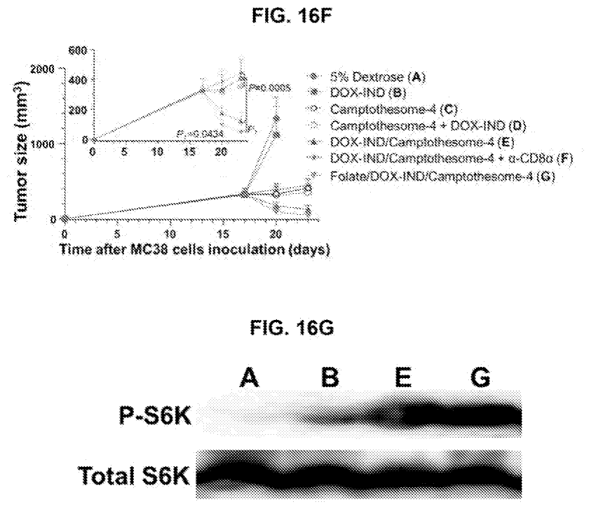

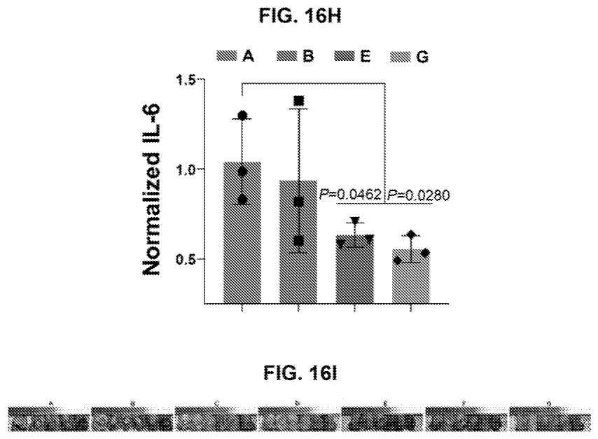

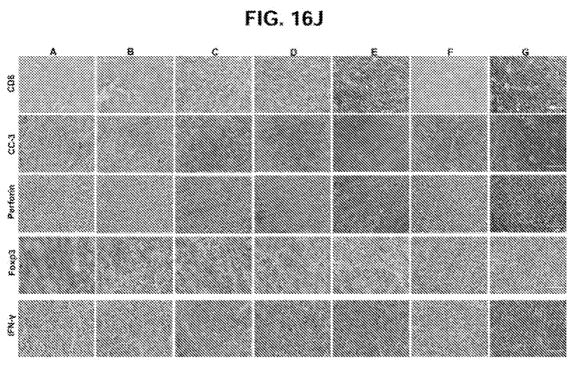

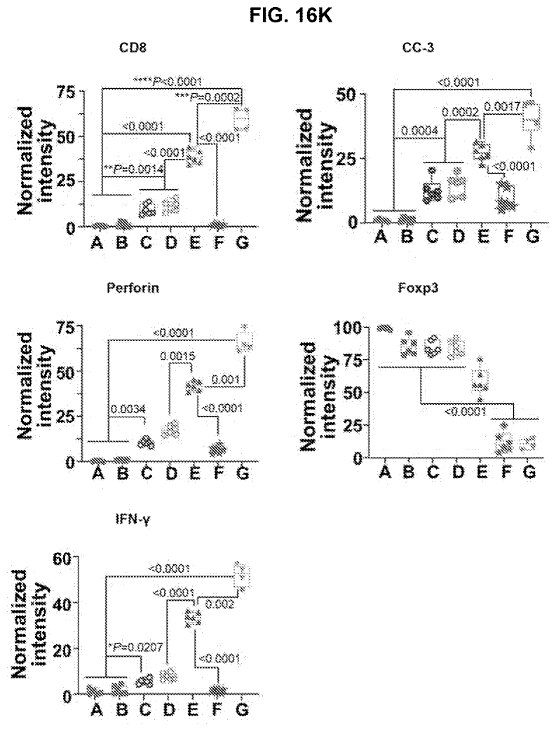

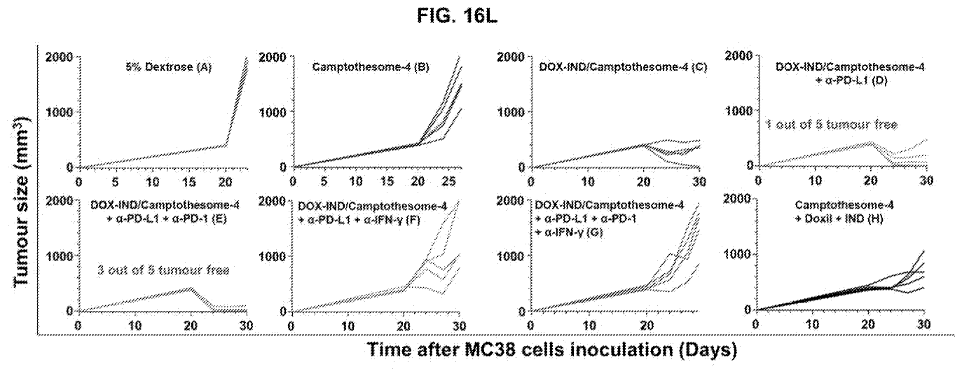

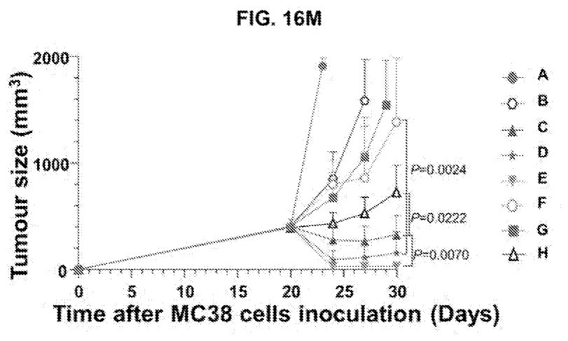

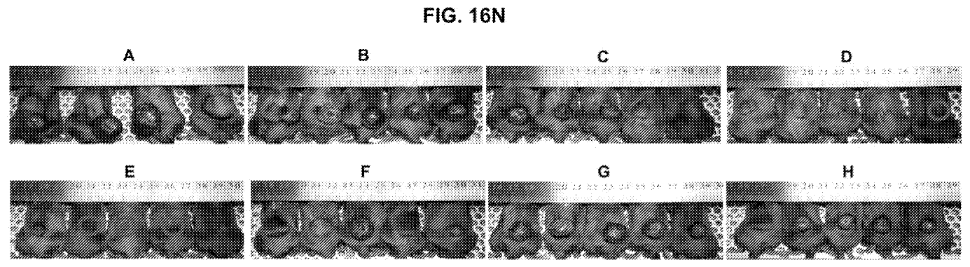

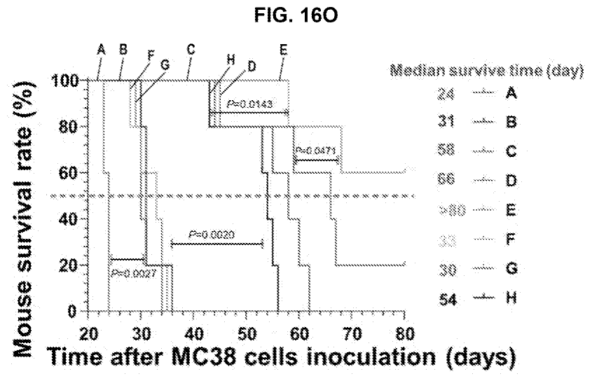

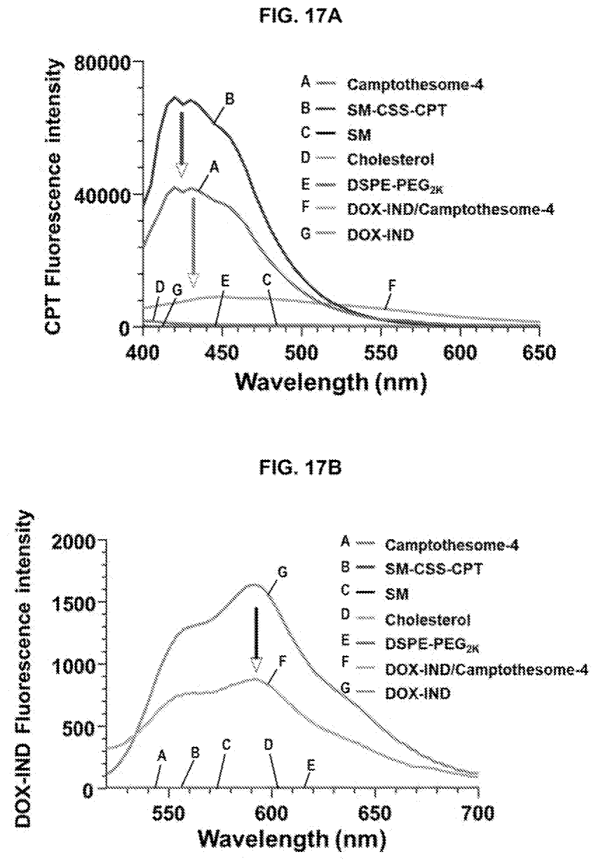

In FIG. 16E-161, 4×10⁵ cells/mouse in 100 μL of serum free medium were SC injected to C57BU6 (n=6). Mice were IV injected once with various treatments at equivalent (eq.) 20 mg CPT/kg and 6.7 DOX-IND mg/kg when tumors grew to ˜300 mm³. α-CD8 (BioXCell, clone 53-6.7) was IP injected at 200 μg/mouse/3 days from day 17. On day 23, mice were euthanized, and tumor tissues were isolated and equally cut into 3 pieces; one part for IHC staining for CD8, Foxp3, Calreticulin, IFN-γ, Perforin, Granzyme B, CC-3) by University of Arizona TACMASR; another two parts for Western blotting of P-S6K and RT-PCR for IL-6, respectively. Mouse weight was monitored every 3 days, and mice survival was monitored every day.

In FIG. 16K-16N, 2×10⁵ cells/mouse were SC injected to C57BL/6 (n=5). When tumors reached ˜400 mm³, mice received a single IV injection with various treatments at eq. 15 mg CPT/kg and 5 mg DOX-IND/kg (IND, 1.7 mg IND/kg; Doxil®, 4.0 mg DOX/kg), or combined with α-PD-L1 or α-PD-L1+α-PD-1, with or without α-IFN-γ. α-PD-L1, α-PD-1, and α-IFN-γ were IP administered as depicted above. Mouse weight was monitored every 3-4 days, and mice survival was monitored every day.

In a separate study, SC MC38 tumor bearing mice (n=3, ˜200 mm³) were IV injected once by Camptothesome-4 (20 mg CPT/kg) with or without α-IFN-γ as described above. 5% dextrose served as the vehicle control. 7 days later, tumors were collected and subject to PD-L1, PD-1, IFN-γ, and IDO1 analysis using IHC staining.

### C. In Orthotopic CT26-Luc (Luciferase-Ex Pressing) Tumor Bearing Mice.

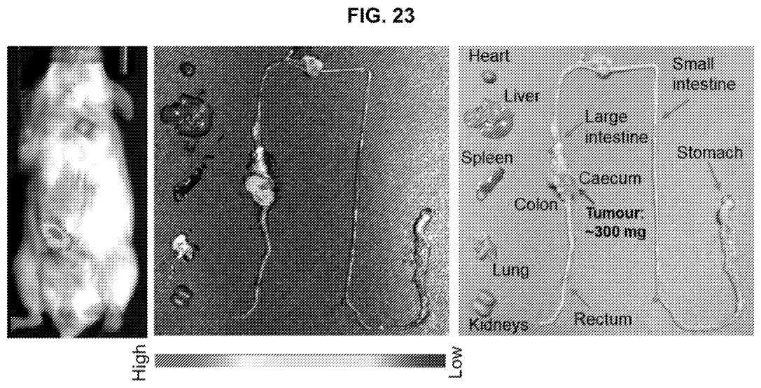

6-8 weeks old BALB/c mice were anesthetized by isoflurane. The hair/fur in abdominal area of mice were removed by a clipper. Then the surgical area underwent three alternating scrubs of betadine/povidone iodine followed by 70% ethanol. A SC injection of buprenorphine SR (1.0 mg/kg) was administered prior to surgery. Afterwards, an abdominal incision (˜1 cm) was created with a sterile disposable scalpel followed by exteriorizing the cecum. 2×10⁶ CT26-Luc cells in 50 μL of RPMI-1640/Matrigel (Corning, Discovery Labware Inc.) (3/1, v/v) were inoculated into the cecum subserosal using a 26-gauge needle (BD precisionGlide™). Cecum was then replaced into the peritoneal cavity after sterilizing the injection site with 70% ethanol to kill cancer cells that may have leaked out [42, 49]. The abdominal wall and skin were closed with size 6-0 absorbable sutures (PDS II, Ethicon) and size 5-0 non-absorbable sutures (PROLENE, Ethicon), respectively. Surgical glue was applied to assure good apposition of skin. Animals were placed on the heating pad during and after surgery and closely monitored until ambulatory, then returned to a clean cage. Tumor burden of a whole mouse body was determined by bioluminescence radiance intensity using Lago optical imaging 10 min after mice were injected IP with 150 mg/kg D-Luciferin (Goldbio, MO, USA). 8 days following cancer cells inoculation (tumor weight: ˜300 mg; FIG. 23), orthotopic CT26-Luc tumor bearing BALB/c mice were randomly allocated into 6 groups (n=6). Mice were then IV administered with a single dose of DOX-IND/Camptothesome-4 (5/15 mg DOX-IND/CPT/kg) with or without folate targeting or combined with IP α-PD-L1+α-PD-1 as described above. Controls included 5% dextrose, α-PD-L1+α-PD-1, and Camptothesome-4. The whole-body tumor burden was monitored using a Lago optical imager on days 8, 11, 15, and 18 and quantified as luminescence radiance intensity (p/sec/cm²/sr) using Aura 3.2.0 imaging software. On day 18, following injection of D-Luciferin, mice were dissected, and gastrointestinal tract and other major organs (heart, liver, spleen, lung, kidneys, stomach, small and large intestines, cecum, and rectum) were quickly obtained and then subject to photographing and ex vivo Lago imaging to investigate the tumor metastasis. Afterwards, tumors were isolated and placed in 4% paraformaldehyde overnight prior to IHC analysis of various immune biomarkers (PD-L1, PD-1, IDO1, CD8, Foxp3, Perforin, Granzyme B, IFN-γ, Calreticulin, LRP1, HMGB1, TLR4, IL-10, IL-12).

### D. In B16-F10-Luc2 Tumor-Bearing Mice.

1×10⁵ B16-F10-Luc2 cells in 100 μL of serum free medium were SC injected in C57BU6 mice (n=5). When tumors reached ˜400 mm³ mice received a single IV administration of DOX-IND/Camptothesome-4 (5/15 mg DOX-IND/CPT/kg) with or without folate targeting or combined with IP α-PD-L1+α-PD-1 as described in orthotopic CT26-Luc tumor model with the same control groups. Tumor burden on mouse whole-body was evaluated by Lago optical imaging on day 14, 17, and 20. On day 20, the tumor metastasis in the digestive tract and other major organs was deciphered, and immune phenotypic analysis (PD-L1, PD-1, IDO1, CD8, Foxp3, calreticulin, LRP1, IFN-γ, granzyme B, and perforin) in orthotopic B16-F10-Luc2 tumors was similarly conducted as mentioned above.

### E. IDO1 Pathway Inhibition in Tumors.

IDO1-mediated immunosuppression entails a series of downstream signaling, such as suppressing mTOR (Mammalian target of rapamycin) and enhancing GCN2 (general control nonderepressible 2) and AHR (Aryl hydrocarbon receptor) pathways (FIG. 15), where phosphorylation of S6K (P-65K) and IL-6 are critically involved [43, 44, 50]. To elucidate the impact of Camptothesome-4-delivered DOX-IND on inhibiting the IDO1, Western blotting for P-6SK and qRT-PCR for IL-6 were carried out as previously reported with a slight modification [43, 44]. Specifically, total S6K was used as the control when determining P-6SK levels. Tumours shown in FIG. 16F were cut into small pieces with scissors and homogenized in RIPA buffer containing a mixture of protein as and phosphatase (250 μl per 50 mg tissue) within 15 min. The lysates were then centrifuged at 12,000 r.p.m. for 10 min, after which equal amounts of proteins in supernatants were loaded onto a 12% Tris-glycine gel (Novex gel, Invitrogen), which was subsequently transferred to a PDVF (polyvinylidene difluoride) membrane. The membrane was blocked by 5% BSA in TBST. This was followed by incubation with the primary antibody (phospho-p70 S6 kinase (Thr389) no. 9205, dilution 1/1000; p70 S6 kinase no. 9202, dilution 1/1000; Cell Signaling) and horseradish peroxidase (HRP)-conjugated secondary antibodies (anti-rabbit IgG, HRP-linked antibody no. 7074, dilution 1/3000; Cell Signaling) that target P-S6K and total S6K. Finally, the blots were developed by soaking the membrane in ECL substrate (Thermo Scientific). Western blot images were acquired using Azure Biosystems software (v. 1.5.0.0518).

For RT-PCR analysis of IL-6 mRNA expression, total RNA was extracted from tumors with miRNeasy Mini Kit (Cat. No. 217004, Qiagen), then treated with RNase-free DNase Set (Cat. No. 79254, Qiagen), and reverse transcribed using SuperScript III First-strand Synthesis System (Cat. No. 18080-051, Invitrogen). Quantitative RT-PCR (qPCR) was conducted with QuantStudio 3 (Thermo Scientific) and PrimeTime probe-based qPCR Assays.

For the IL-6 Assay, ID Mm.PT.58.10005566 primers (Integrated DNA Technologies) were used:

(1)
SEQ ID NO: 1
5′-AGCCAGAGTCCTTCAGAGA-3′
(2)
SEQ ID NO: 2
5′-TCCTTAGCCACTCCTTCTGT-3′

For the GAP D H Assay, ID Mm.PT.39a.1 primers (Integrated DNA Technologies) were used:

(1)
SEQ ID NO: 3
5′-AATGGTGAAGGTCGGTGTG-3′
(2)
SEQ ID NO: 4
5′-GTGGAGTCATACTGGAACATGTAG-3′

PCR was carried out as follows: 3 min at 95° C., followed by 40 cycles at 95° C. for 5 s, 60° C. for 30 s.

### F. Statistical Analyses.

The level of significance in all statistical analysis was set at a probability of P<0.05. Data are presented as mean±standard deviation (SD) and analyzed by one-way analysis of variance (ANOVA) followed by Tukey's post hoc test using Prism 8.0 (GraphPad Software). Comparison of Kaplan-Meier survival curves was performed with the Log-rank Mantel-Cox test.

### Example 4—Establishing the Camptothesome Nanotherapeutic Platform

The following example provides further evidence demonstrating the efficacy of nanovesicles of the present disclosure in the treatment and/or prevention of cancer.

Despite enormous therapeutic potential of immune checkpoint blockade (ICB), it benefits only a small subset of patients. Some chemotherapeutics switch tumors from “immune-cold” to ‘immune-hot’ to potentiate ICB. However, a safe/robust platform implementing favorable immune effects to synergize with ICB remains scarce.

The following example describes a sphingomyelin (SM)-derived CPT liposomal nanotherapeutic platform with different tumor-sensitive linkages (ester, glycine, and disulfide bonds) and varied linker length. SM, a naturally occurring phospholipid and major component in high-density lipoprotein and animal cell membrane, contains a hydroxyl group, enabling conjugation to functional moieties (e.g., hydroxyl/carboxylate groups) of therapeutics. SM-CPTs self-assembled into Camptothesomes in aqueous medium driven by SM's amphiphilicity, enhancing lactone stability. Structure activity relationship analysis identified the disulfide-bridged conjugate with a longer linker (SM-CSS-CPT, Camptothesome-4) outperformed free CPT and other counterparts, exhibiting increased circulation half-life, enhanced tumor uptake, no overt side effects, deep tumor penetration and efficient intratumoral CPT release. Camptothesome-4 excelled Onivyde® (a liposomal irinotecan-CPT derivative) by bolstering tumor reduction and prolonging mice survival. Furthermore, Camptothesome-4 significantly induced tumor-infiltrated CD8, Granzyme B, Perforin, IFN-γ, and cleaved caspase-3 (CC-3) in CRC tumors, demonstrating CTL-elicited antitumor immunity. The increased IFN-γ upregulated intratumoral PD-L1/PD-1 expression, which has been associated with improved response to ICB [13]. These findings justified combining PD-L1/PD-1 blockade with Camptothesome-4, leading to eradication of MC38 tumors in 83.3% immunocompetent mice.

Sphingomyelin (SM), a naturally occurring sphingolipid in animal cell membranes, is the second most abundant lipid and major component of High Density Lipoprotein and the plasma membrane(ref). SM is hydrolyzed by sphigomyelinase with the PC head group released into the aqueous environment while the ceramide diffuses through the membrane, which plays an essential role in the apoptotic signaling pathway. Like other phospholipids (PL), SM is an amphiphilic lipid with a polar PC head group and two aliphatic acyl chains, which allows it to self-assemble to a liposome in aqueous medium. In contrast to the two ester-bonded diacyl lipid chains in other PL, SM only contains one amide-bridged acyl lipid. The amide linkage is more stable than ester bond under physiological condition and acidic environment; in addition, SM is also more prone to intermolecular hydrogen bonding than other PL, both of which make SM less susceptible to hydrolysis or enzymatic degradation than PL carrying ester bond, rendering higher liposome stability and improved PK/drug retention during circulation. So far, there are 15 FDA-approved cancer liposomal nanoformulations including SM liposomes. SM contains a functional —OH, which makes it possible to be conjugated to therapeutic molecules with functional moieties such as —COOH, —OH, —NH₂ and/or C═O through linker moieties as described above and non-limited to linkers comprising a labile ester, glycine or disulfide bond.

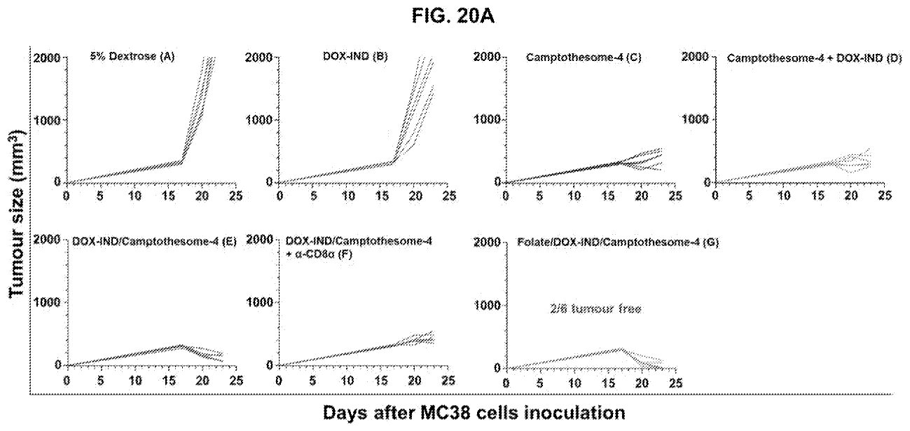

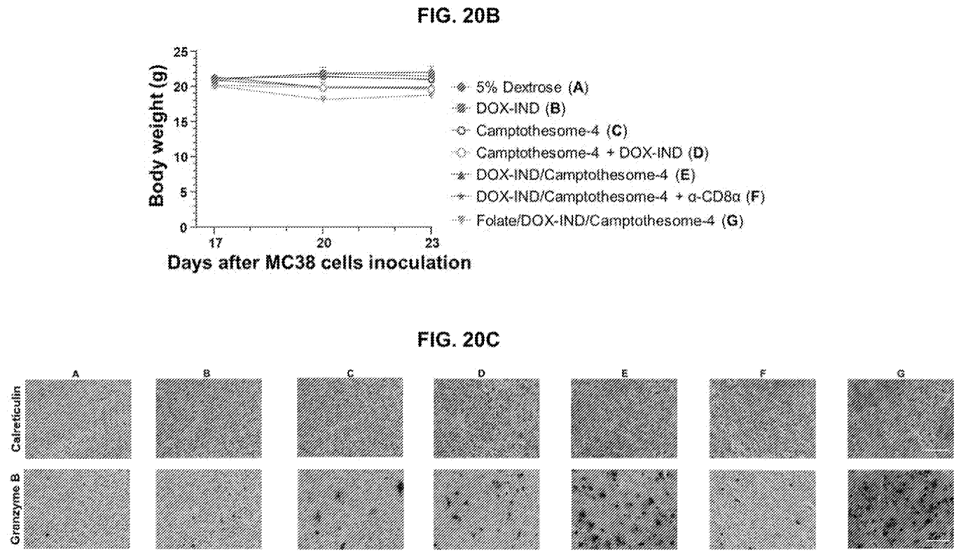

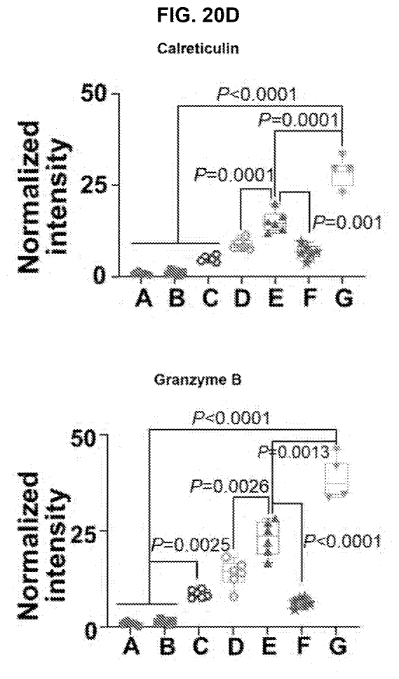

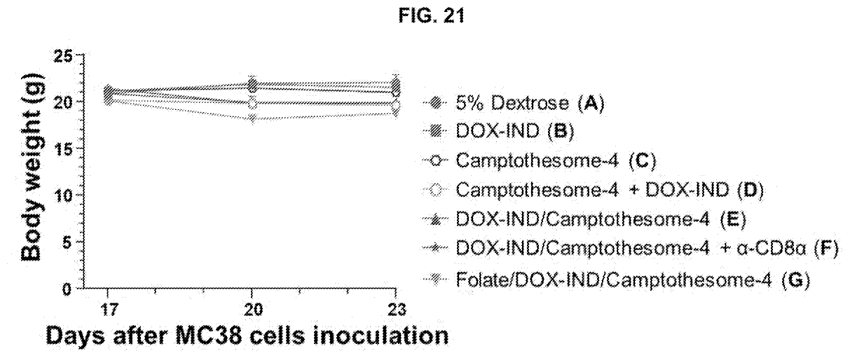

Indoleamine 2,3-dioxygenase (ID01), another independent crucial immune checkpoint, enzymatically degrades tryptophan, rendering CTL anergy while activating regulatory T cells (Tregs), resulting in immunosuppression in various tumors [14-16]. Consistent with literature [17, 18], IDO1 expression in CRC tumors was confirmed, which was increased in response to INF-γ production (FIGS. 20 and 21). To reverse IDO1-mediated immunosuppression, it was proposed to co-deliver IND (due to its safety/potency) [15, 19] with Camptothesome-4. However, IND's limited solubility rendered direct loading challenging. To tackle this, Indoximod (IND) was conjugated to immunogenic cell death (ICD) inducer-Doxorubicin (DOX), using DOX as a membrane-crossing carrier to import IND into Camptothesomes. A pH-sensitive hydrazone linkage was specifically designed so that DOX-IND breaks inside nanovesicle under protonating agent-produced acidic pH, forming drug precipitates incapable of back diffusion across lipid bilayer [20]. IND has been reported to synergize with DOX to elicit tumor regression [21]. With ICD-eliciting potential, DOX offers additional antitumor immunity benefits [22]. Strikingly, DOX-IND/Camptothesome-4 cured a significant portion of mice bearing advanced metastatic orthotopic CRC or late-stage subcutaneous (SC) CRC/melanoma tumors when functionalized with folate tumor targeting and/or combined with PD-L1/PD-1 inhibitors.

Sphingomyelin-derived cam ptothecin nanovesicle (Camptothesome) improved pharmacokinetics/antitumor efficacy (VS CPT) with deep tumor penetration/no systemic toxicities, while triggering Granzyme B/Perforin-mediated cytotoxic T lymphocytes (CTL) immunity. Co-blocking PD-L1/PD-1, one-time intravenous administration of Camptothesome eradicated established MC38 colorectal adenocarcinoma in 83.3% mice with developed memory T cell effects. These antitumor effects were correlated with enhanced tumor-infiltrated CTL and significantly attenuated by IFN-γ neutralization/CD8 depletion. Co-encapsulation of indoleamine 2,3-dioxygenase (IDO1) inhibitor-indoximod into Camptothesomes using immunogenic cell death (ICD) inducer-doxorubicin as a transmembrane-enabling agent eliminated 40-66.7% tumors in orthotopic CRC (˜300 mg) or melanoma (˜400 mm³) murine models with complete metastasis remission when combined with PD-L1/PD-1 co-blockade/folate targeting.

### Self-Assembly of Camptothesomes

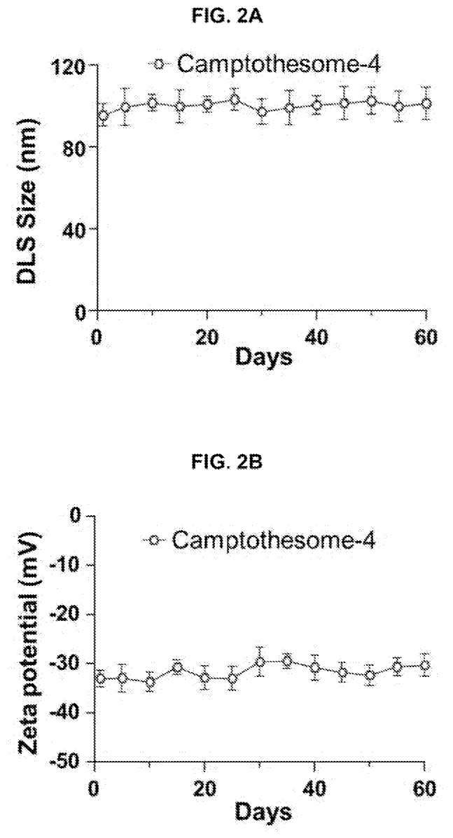

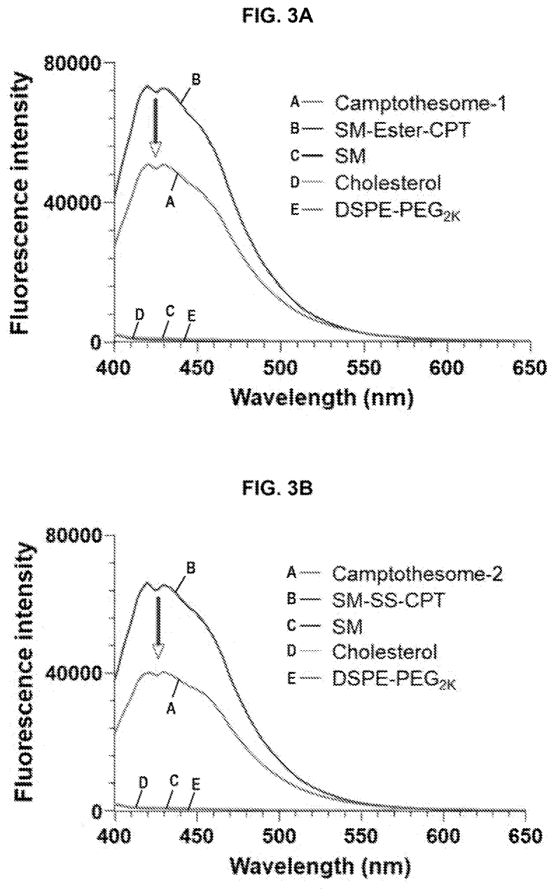

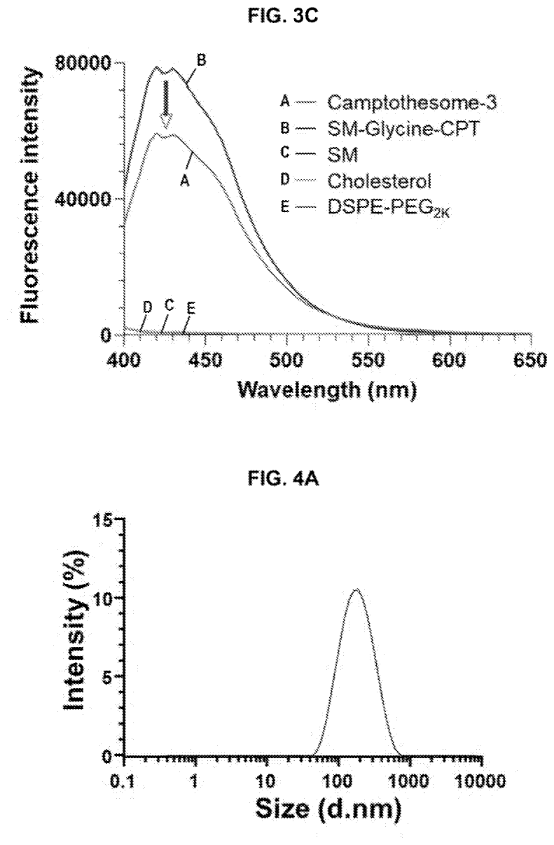

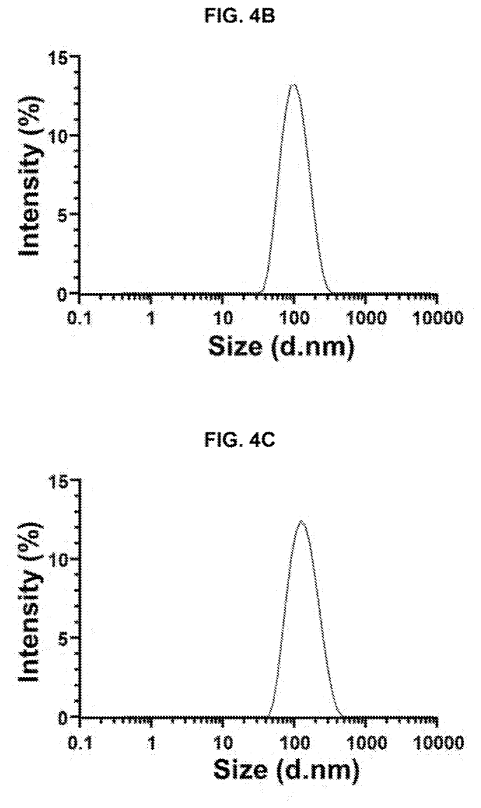

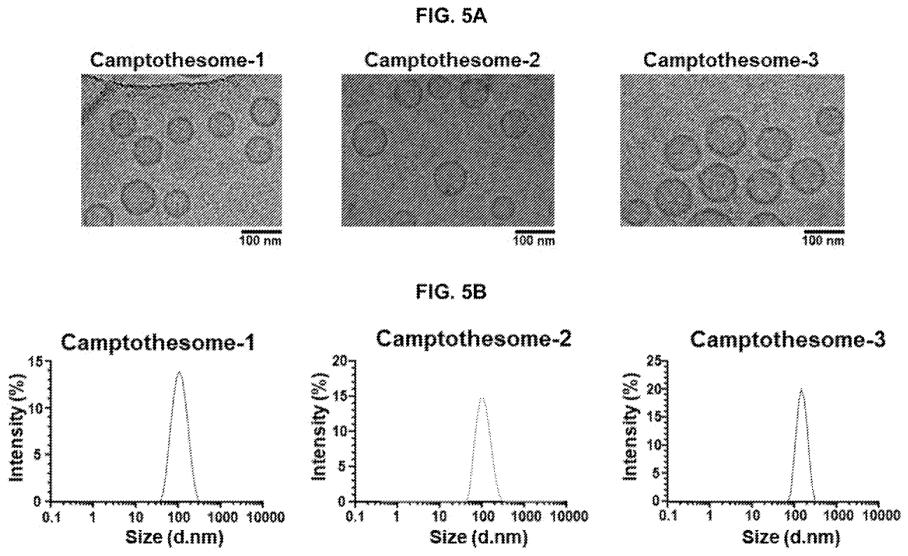

Four different SM-CPTs were initially synthesized (FIG. 1 and Table 3)-one with an ester bond (SM-Ester-CPT), one with a glycine bond (SM-Glycine-CPT), one with a disulfide linkage (SM-SS-CPT), and one with a disulfide linkage and a longer linker (SM-CSS-CPT). These linkages are sensitive to high hydrolase, cathepsin B, and glutathione levels, respectively, in tumor tissues/cells [23-26]. Their chemical structures were confirmed by ¹H-NMR, ¹³C-NMR, and ESI-MS. A longer linker was used to link disulfide bond with SM by introducing additional alkyl groups to form a non-carbonate ester to prevent spontaneous pyrolysis, a process that leads to premature carbonate ester bond breakdown and CO₂ release (Scheme 2) [27]. All SM-CPTs efficiently self-assembled into liposomal nanovesicles (Camptothesomes) visualized by cryo-EM (FIG. 1C; FIG. 5). In addition, the self-assembly of SM-CPTs was substantiated using ¹H-NMR. The typical proton spectrum was shown for SM-CPTs, SM, Cholesterol, DSPE-PEG2K in CDCl₃ due to their free dispersion in this solvent. Camptothesomes in CDCl₃ expressed all typical proton signals for each individual constituent. However, when collected in D₂O, the proton signals from individual components in Camptothesomes were all suppressed, attributing to the spontaneous self-assembly of different lipids into Camptothesomes, which disrupted their free dispersion. The CPT's proton signals in SM-CPTs in CDCl₃ disappeared from Camptothesomes when dissolved in D₂O, suggesting successful packaging of CPT into lipid bilayer. Additionally, SM-CPTs' fluorescence was significantly quenched upon incorporation into Camptothesomes, indicating the strong π-π stacking interaction among SM-CPT molecules (CPT contains several aromatic rings) and further corroborating the self-assembly process (FIG. 1D; FIG. 3). All Camptothesomes displayed narrow size distribution reflected by low polydispersity and enhanced CPT lactone stability (FIG. 1E-1F; FIG. 5B). Camptothesome-4 had a smaller size and significantly longer formulation stability as compared to Camptothesome-2 and Camptothesome-3 (FIG. 1G) and remained stable for up to 2 months as evidenced by no significant size and zeta potential change (FIG. 2).

TABLE 3
Physicochemical characterizations of four Camptothesomes

| | | | | | | DLS | | |
|---|---|---|---|---|---|---|---|---|
| Camptothesomes | | | | DSPE- | CPT | Size by | Zeta | |
| (respective | SM | SM-CPT | Cholesterol | PEG2K | DLC | intensity | Potential | |
| SM-CPT used) | (Molar %) | (Molar %) | (Molar %) | (Molar %) | (%) | (nm) | (mV) | Polydispersity |
| Camptothesome-1 | 50.5 | 31.6 | 13.3 | 4.6 | 12.3 | 99.8 ± 8.34 | −38.4 ± 3.57 | 0.174 ± 0.035 |
| (SM-Ester-CPT) | | | | | | | | |
| Camptothesome-2 | 62.9 | 19.8 | 12.9 | 4.4 | 7.9 | 102.4 ± 5.69 | −22.4 ± 4.62 | 0.163 ± 0.028 |
| (SM-SS-CPT) | | | | | | | | |
| Camptothesome-3 | 67.3 | 16.1 | 12.3 | 4.3 | 6.1 | 137.9 ± 10.1 | −15.6 ± 3.40 | 0.119 ± 0.053 |
| (SM-Glycine-CPT) | | | | | | | | |
| Camptothesome-4 | 68.3 | 14.8 | 12.6 | 4.3 | 6.1 | 93.1 ± 7.63 | −31.7 ± 2.73 | 0.173 ± 0.026 |
| (SM-CSS-CPT) | | | | | | | | |

### Safety and Toxicological Profile of Camptothesomes

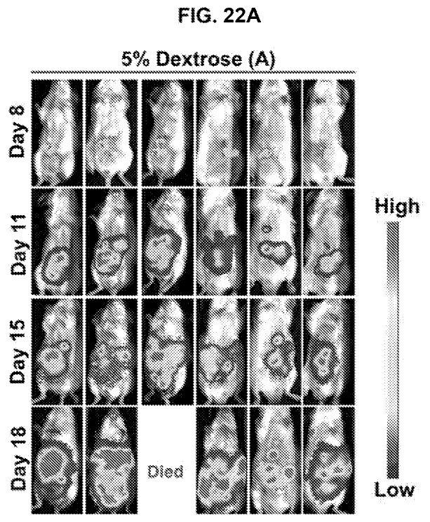

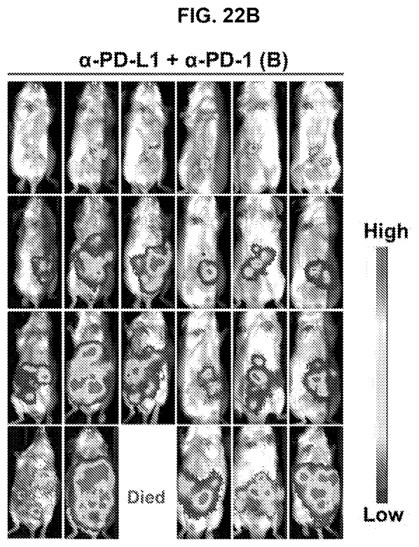

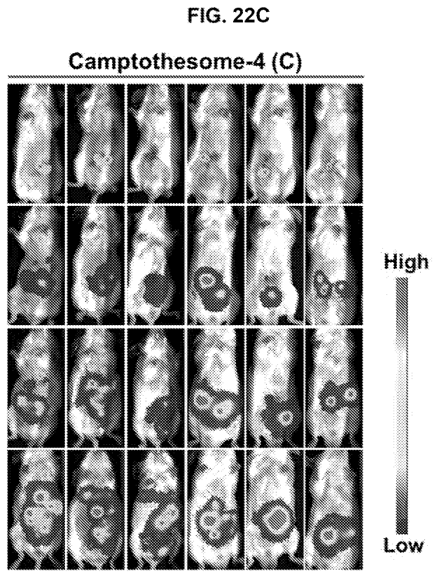

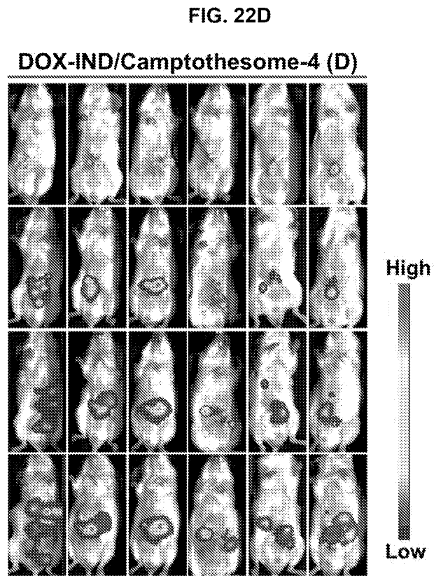

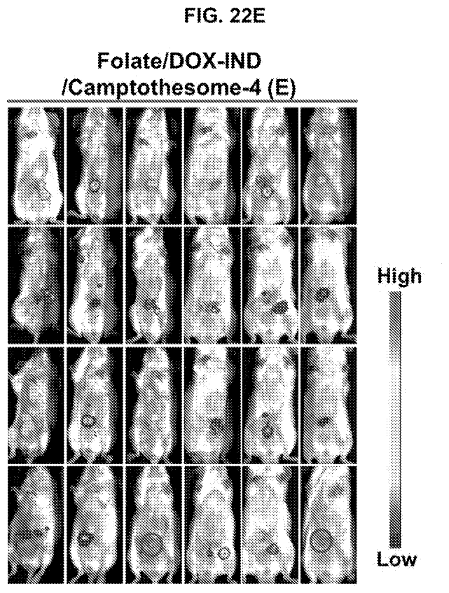

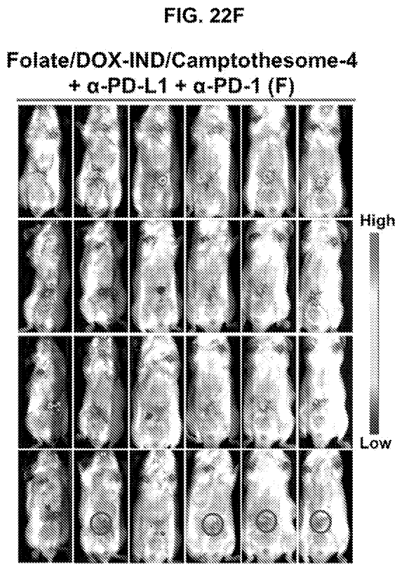

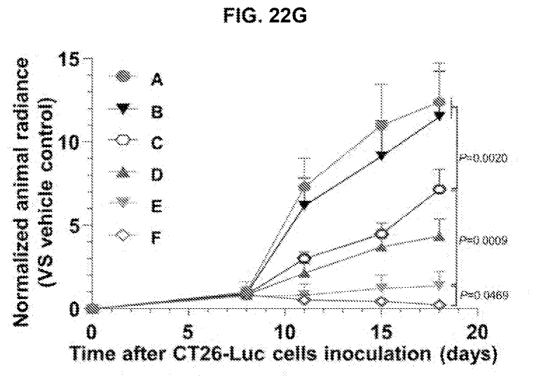

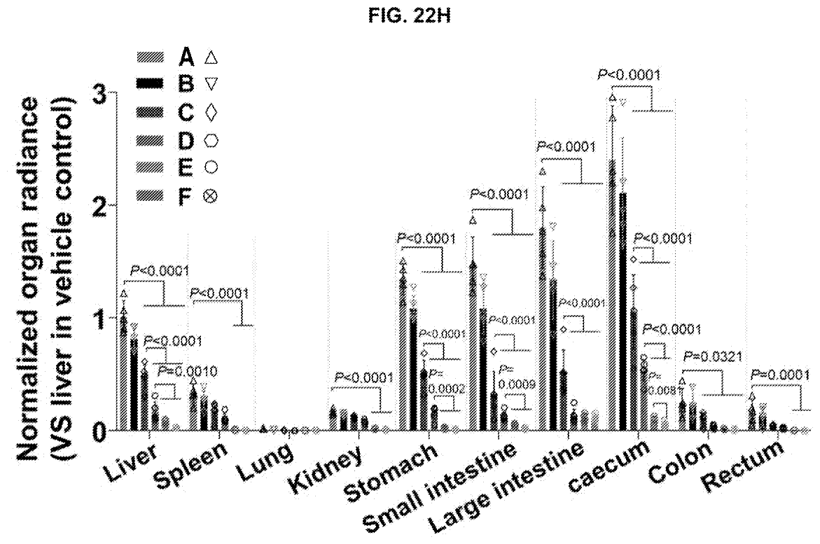

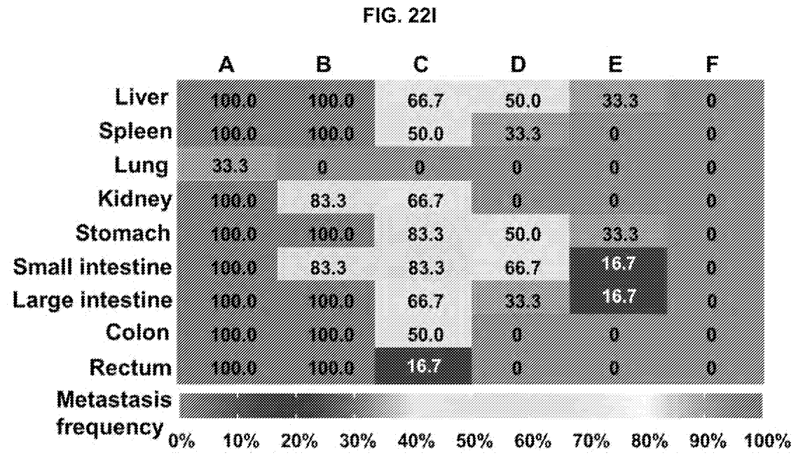

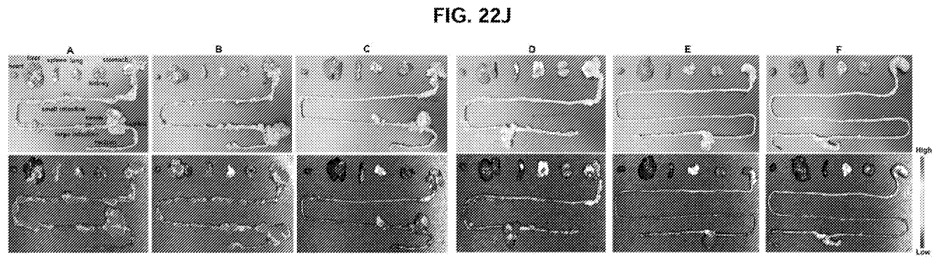

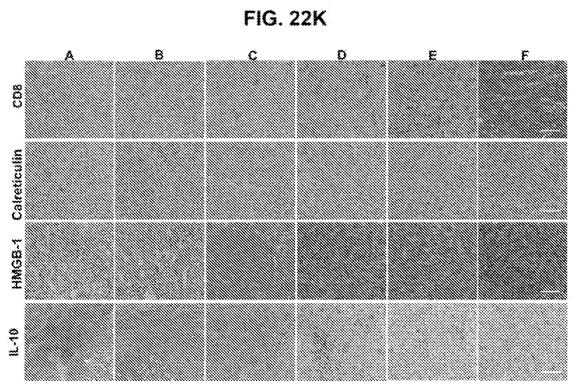

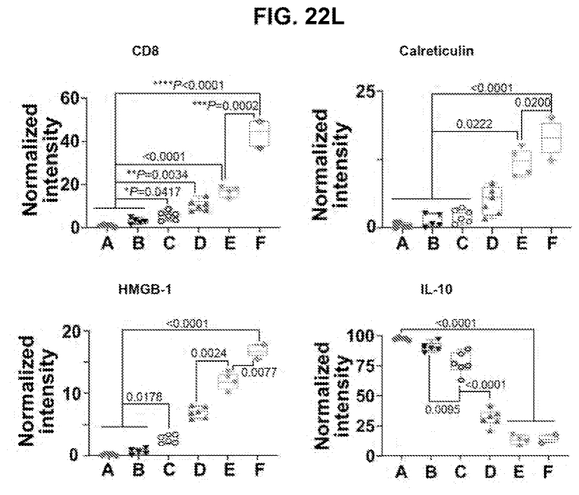

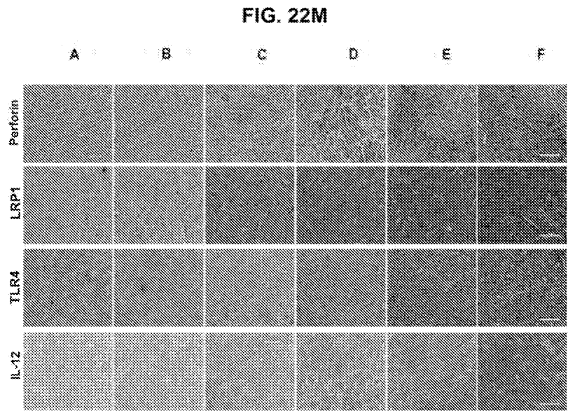

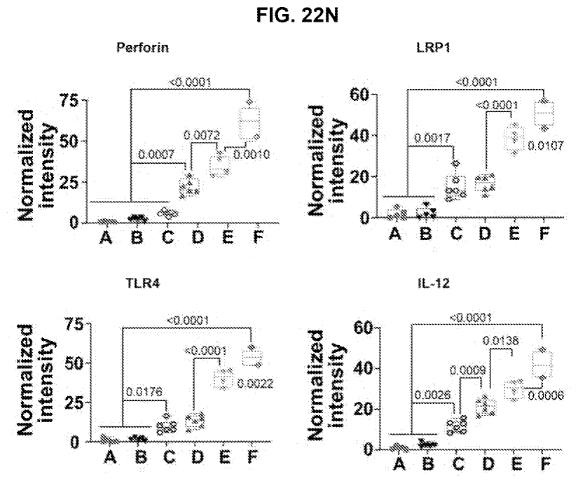

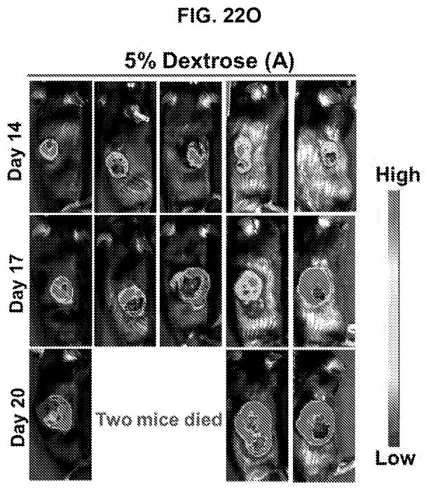

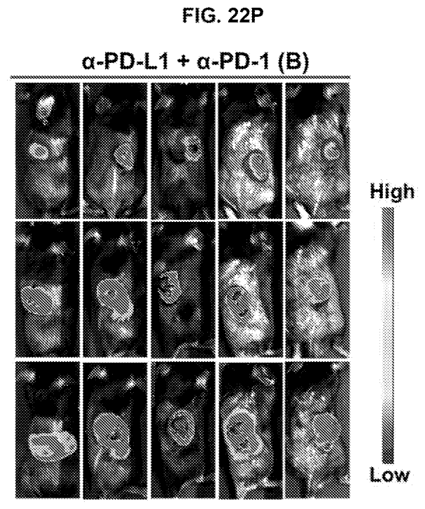

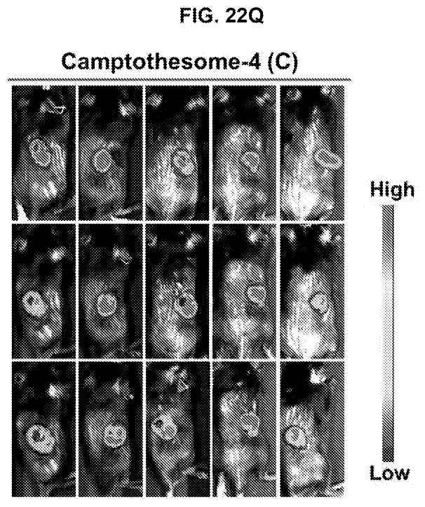

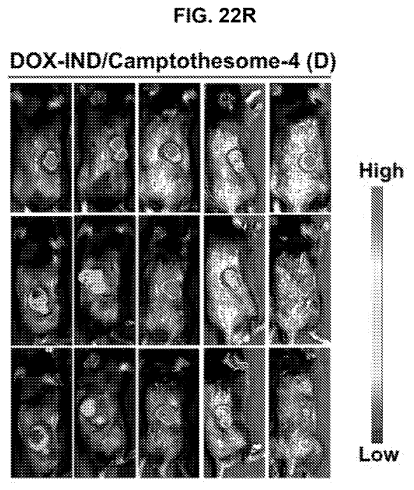

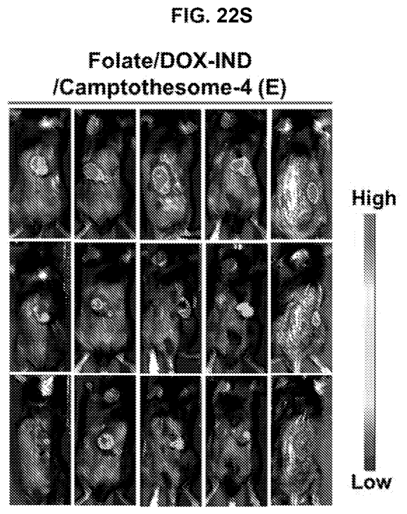

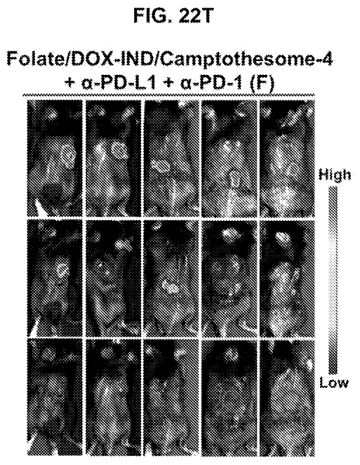

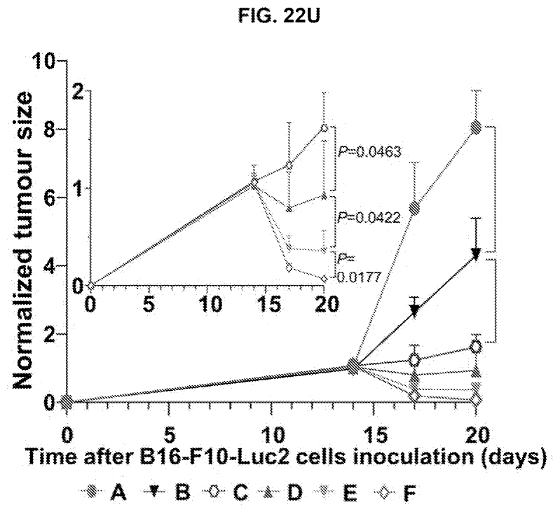

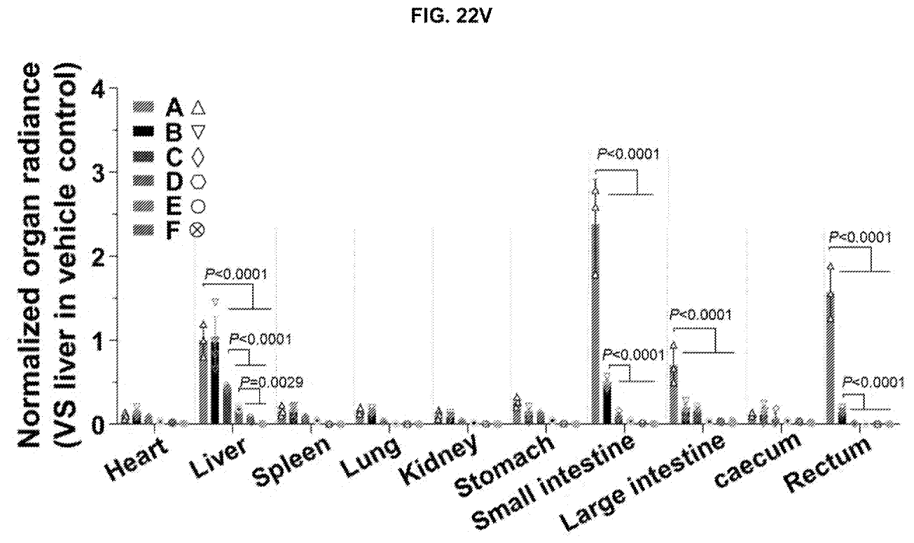

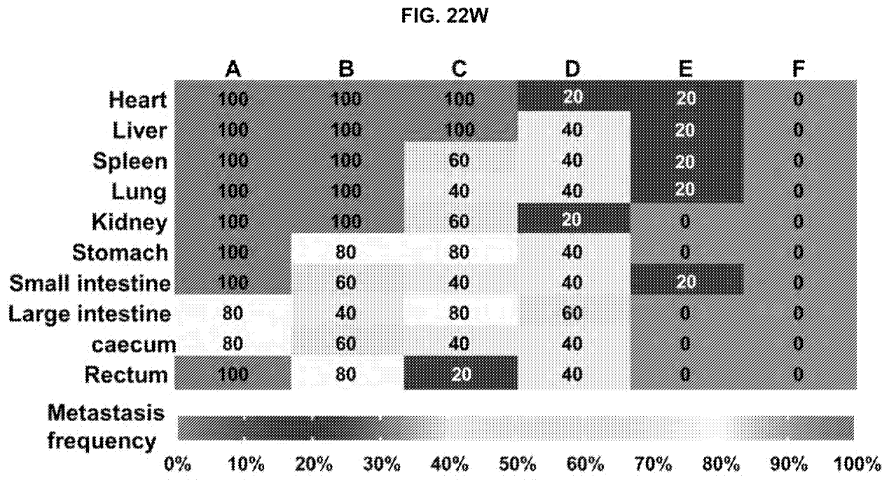

The MTD of 4 different Camptothesomes were evaluated in healthy BALB/c mice following an IV injection at various doses. Free CPT served as a control. Camptothesomes improved MTD of CPT (5 mg/kg) by 6-24-fold (30-120 mg CPT/kg) without adverse effects to healthy tissues, immune cells (leukocytes), red blood cells, and thrombocytes (FIG. 22H-22J; Table 4). While CPT at MTD did not cause significant mouse weight loss, it entailed severe systemic toxicities by significantly deviating the alkaline phosphatase, alanine transaminase, blood urea nitrogen, creatinine, glucose, and total proteins levels from normal values, markedly decreased lymphocytes counts and hemoglobin concentration, and induced overt hepatic steatosis and diffuse microvesicular degeneration of hepatocytes in hepatic tissue and hemorrhage in interstitial tissue in kidneys (FIG. 22K-22N). These data demonstrate the remarkable in vivo safety profile of Camptothesomes and their potential to maximize therapeutic efficacy of CPT against tumors.

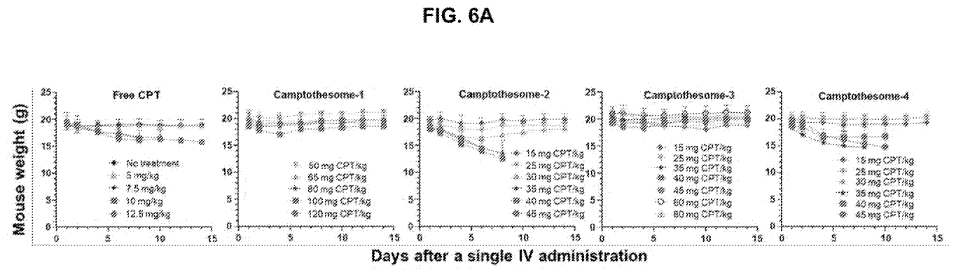

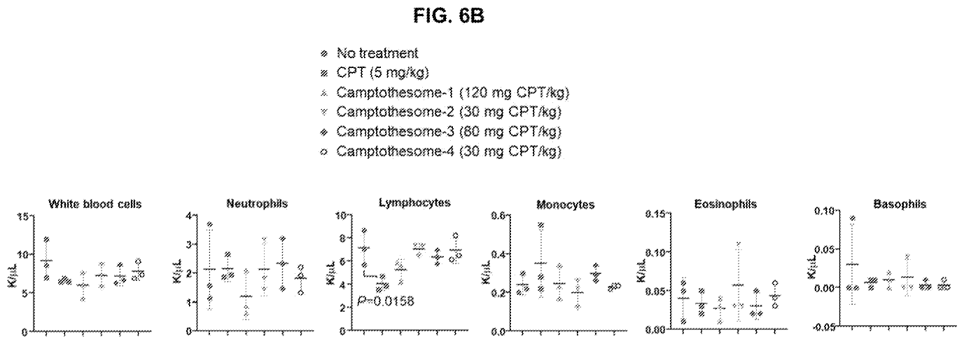

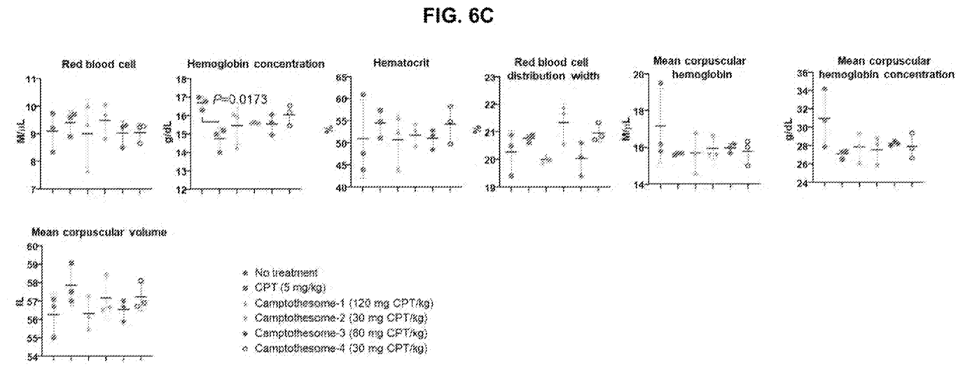

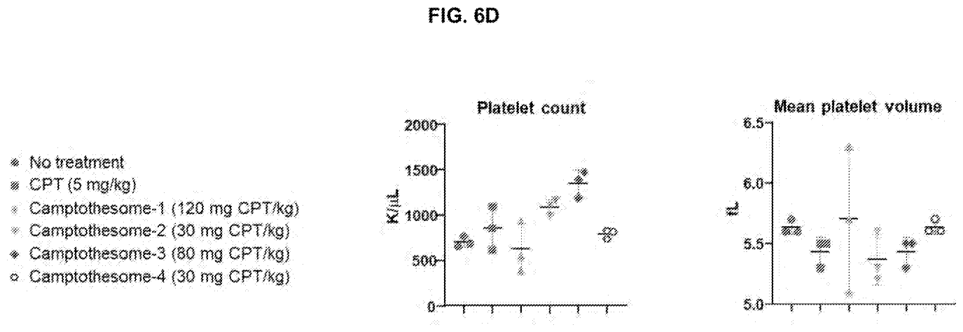

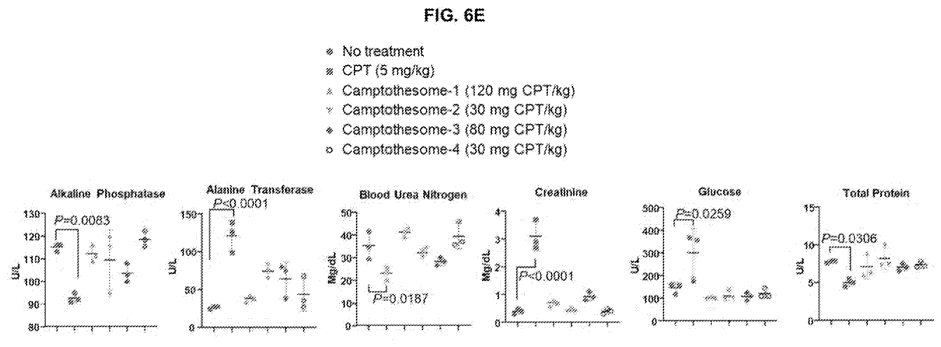

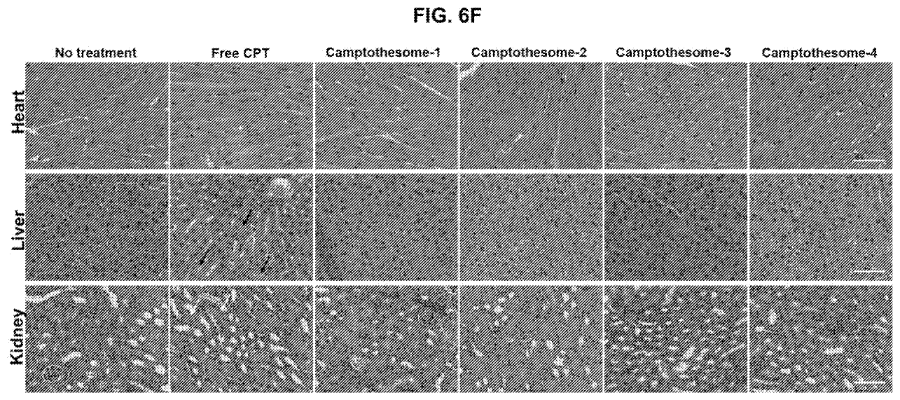

TABLE 4
MTD Investigation as shown in FIG. 6 for free CPT, Camptothesome-1,
Camptothesome-2, Camptothesome-3, and Camptothesome-4.

| Formulation | Dose (mg/kg) | Animal Death | Weight Loss (%) |
|---|---|---|---|
| Free CPT | 5 | 0/3 | 1.82 |
| | 7.5 | 1/3 | N/A |
| | 10 | 1/3 | N/A |
| | 12.5 | 0/3 | 25.35 |
| Campothesome-1 | 50 | 0/3 | −2.62 |
| | 65 | 0/3 | 4.24 |
| | 80 | 0/3 | −3.56 |
| | 100 | 0/3 | 0.39 |
| | 120 | 0/3 | 0.46 |
| Campothesome-2 | 15 | 0/3 | 0.31 |
| | 25 | 0/3 | 0.51 |
| | 30 | 0/3 | 11.3 |
| | 35 | 2/3 | N/A |
| | 40 | 2/3 | N/A |
| | 45 | 3/3 | N/A |
| Campothesome-3 | 15 | 0/3 | 3.87 |
| | 25 | 0/3 | 0.57 |
| | 35 | 0/3 | 1.78 |
| | 40 | 0/3 | 1.55 |
| | 45 | 0/3 | 2.98 |
| | 60 | 0/3 | −0.03 |
| | 80 | 0/3 | −2.99 |
| Campothesome-4 | 15 | 0/3 | 2.23 |
| | 25 | 0/3 | 3.33 |
| | 30 | 0/3 | 0.89 |
| | 35 | 1/3 | N/A |
| | 40 | 1/3 | N/A |
| | 45 | 2/3 | N/A |

### Pharmacokinetics, Biodistribution, and Tumor Penetration

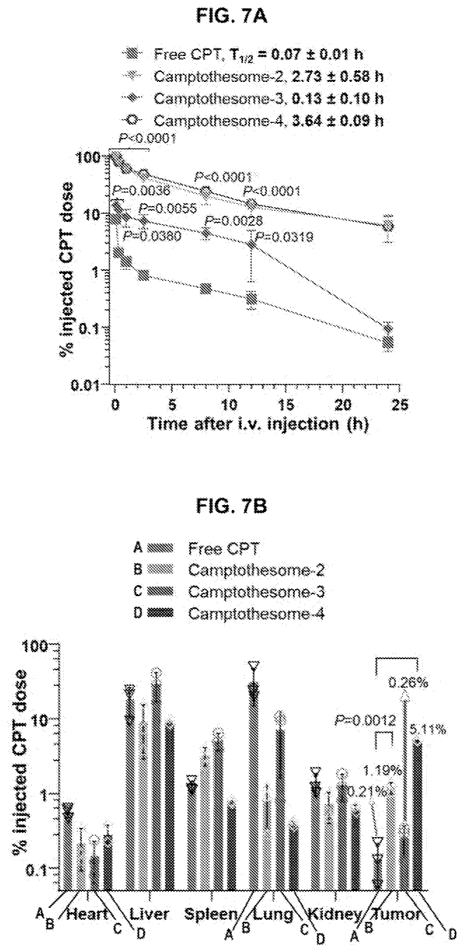

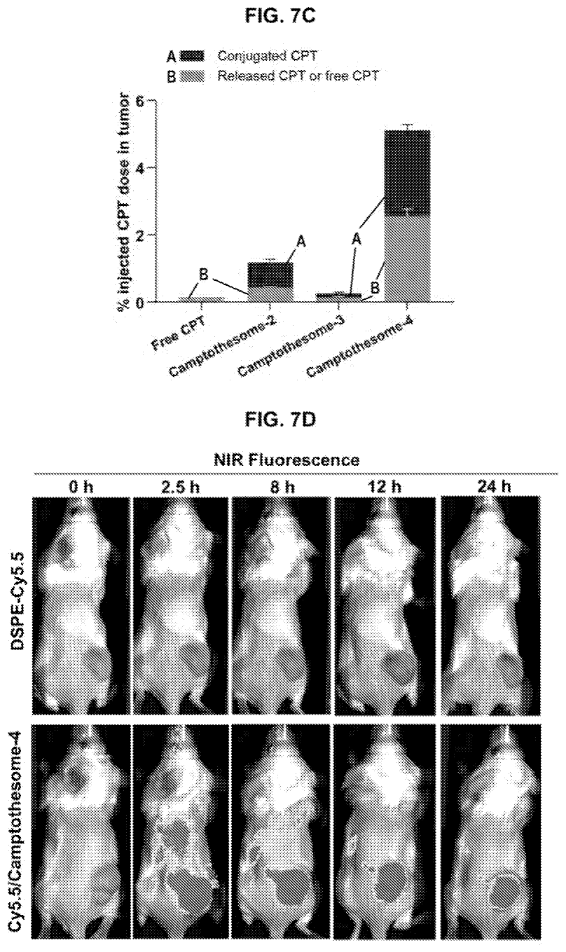

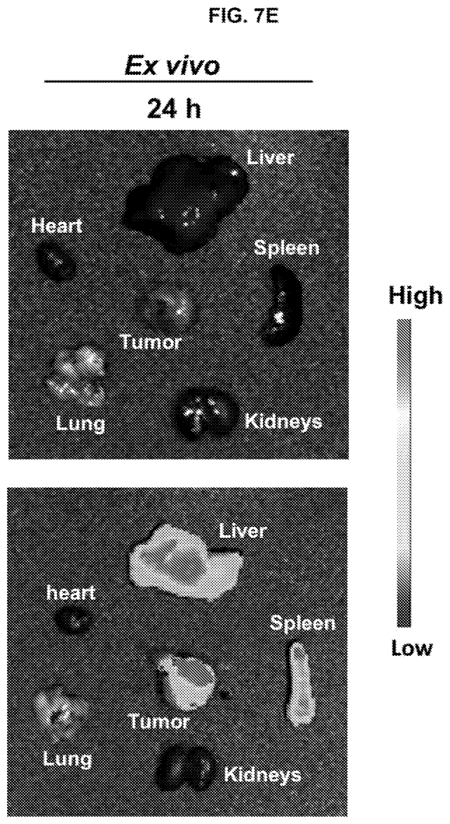

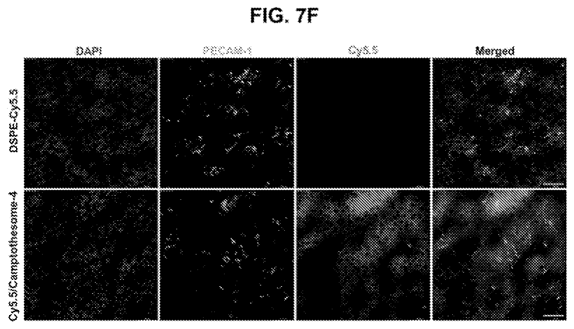

The pharmacokinetics and tissue distribution for Camptothesomes were evaluated in CT26 tumor-bearing mice. Free CPT was rapidly eliminated from blood, while Camptothesomes significantly prolonged the circulation half-life and delivered more drug into tumors with efficient CPT release (FIG. 7A-7C), affirming the hypothesis that tumor sensitive linkages bridging SM and CPT can be readily cleaved in tumors. Camptothesome-4 reaped the highest tumor uptake by delivering 24.3-fold more drug into tumors, 50.2% of which was converted into free CPT (FIG. 16B). The 5.11% injected dose in tumor for Camptothesome-4 is higher than most of FDA-approved nanomedicines (2-3%) in mice [34-37]; nonetheless, Camptothesome-4 had lower distribution to heart, liver, spleen, lung, and kidneys, thereby minimizing non-specific systemic toxicities (FIG. 16A). The tumor delivery efficiency of Camptothesome-4 in real-time live animals bearing CT26 tumor was then evaluated. Camptothesome-4 was labeled with a near infrared dye, 1,2-dioleoyl-sn-glycero-3-phosphoethanolamine-N-(cyanine 5.5), (DSPE-Cy5.5) and tested various amounts to ensure the dye-doped nanoparticle resembled parental Camptothesome-4. At a 0.2 weight % of total lipids, Cy5.5/Camptothesome-4 had almost identical size and zeta potential as those of original nanovesicle without dye. Free DSPE-Cy5.5-injected m ice exhibited no observable signal in tumors during entire monitored period. In stark contrast, Cy5.5/Camptothesome-4 peaked in tumors as early as 2.5 h and retained significant fluorescence intensity after 24 h post IV administration (FIG. 7D), according with ex vivo autopsies imaging (FIG. 7E). To unravel whether Camptothesome-4 can extravasate and penetrate deeply into tumors following tumor delivery, platelet endothelial cell adhesion molecule-1 (PECAM-1) was stained to visualize tumor vasculature (FIG. 7E, green). The green immunofluorescence exhibited extensive distribution of blood vessels in CT26 tumors. Interestingly, the red fluorescence signals from Cy5.5/Camptothesome-4 distributed throughout the tumor section at 24 h post IV injection, suggesting its deep tumor penetration capability (FIG. 7E) that is crucial for antitumor efficacy.

TABLE 5
Pharmacokinetics Parameters for Free CPT, Camptothesome-2,
Camptothesome-3, and Camptothesome-4 (see FIG. 7A)

| PK Parameter | Free CPT | Camptothesome-2 | Camptothesome-3 | Camptothesome-4 |
|---|---|---|---|---|
| T_{1/2} (h) | 0.07 ± 0.01 | 2.73 ± 0.58 | 0.13 ± 0.10 | 3.64 ± 0.09 |
| V (μg/μg/mL) | 26.44 ± 0.27 | 1.13 ± 0.04 | 5.30 ± 2.00 | 1.15 ± 0.03 |
| CL (μg/μg/mL/h) | 36.06 ± 5.00 | 0.29 ± 0.05 | 1.18 ± 0.47 | 0.22 ± 0.01 |
| AUC₀₋ₜ (μg/mL · h) | 10.95 ± 1.55 | 1378.24 ± 243.30 | 329.04 ± 93.03 | 1810.72 ± 43.90 |
| AUC_{0-∞} (μg/mL · h) | 11.25 ± 1.52 | 1382.89 ± 247.50 | 377.09 ± 153.6 | 1829.81 ± 45.62 |
| AUMC (μg/mL · h²) | 65.42 ± 6.99 | 5586.67 ± 2057.51 | 4277.30 ± 2086.16 | 9618.86 ± 438.55 |
| MRT (h) | 5.85 ± 0.41 | 3.94 ± 0.84 | 9.95 ± 5.57 | 5.25 ± 0.14 |
| Vₛₛ (μg/μg/mL) | 211.68 ± 38.45 | 1.13 ± 0.05 | 10.41 ± 3.03 | 1.15 ± 0.03 |

TABLE 6
Physicochemical characterizations of Cy5.5/Camptothesome-4 with regards to size,
zeta potential, and polydispersity using different molar ratios of DSPE-Cy5.5

| | DSPE-Cy5.5 | Z-Average Size | Zeta Potential | |
|---|---|---|---|---|
| Formulation | (% wt) | (d, nm) | (mV) | Polydispersity |
| Cy5.5/Camptothesome-4 | 0.1 | 169.1 ± 10.51 | −28.9 ± 2.50 | 0.204 ± 0.047 |
| Cy5.5/Camptothesome-4 | 0.2 | 95.5 ± 4.12 | −31.0 ± 3.28 | 0.167 ± 0.022 |
| Cy5.5/Camptothesome-4 | 0.3 | 118.3 ± 5.76 | −28.7 ± 1.00 | 0.210 ± 0.034 |

### Camptothesome-Elicited CTL Immunity was IFN-γ-Dependent and Potentiated PD-L1/PD-1 Blockade to Eradicate CRC Tumors.

To elucidate if Camptothesomes retained the anticancer activity of CPT, CT26 tumor-bearing mice were treated with a single dose of Camptothesomes in comparison to free CPT and Onivyde®. Tumors in vehicle control mice grew uncontrollably, demonstrating its aggressive nature; free CPT, Onivyde®, Camptothesome-1, and Camptothesome-3 presented significant tumor reduction (FIG. 8A). Two disulfide-bonded Camptothesomes enhanced tumor inhibition, particularly in Camptothesome-4 that further prolonged mice survival rate (FIG. 8A-8B). This is likely attributed to the higher chemical and formulation stability and increased tumor uptake (Schemes 2, 4; FIG. 1G; FIG. 7B). Since Camptothesome-4 outperformed other Camptothesomes in terms of stability, tumor uptake, and efficacy, it was selected from subsequent therapeutic and immune investigation. Interestingly, PD-L1, PD-1, and IFN-γ were markedly upregulated in mice tumor tissues following Camptothesome-4 administration, and PD-L1 and PD-1 induction was dictated by IFN-γ as depleting IFN-γ entailed significantly dampened PD-L1 and PD-1 expression (FIG. 8C). High PD-L1/PD-1 expression was associated with improved response to PD-L1/PD-1 blockade19. Thus, these data provide solid grounds for combining Camptothesome-4 with PD-L1/PD-1 blockade for treating CRC. A single IV administration of Camptothesome-4 plus three times of IP injected anti-PD-L1 monoclonal antibody (α-PD-L1) to CT26 tumor-bearing mice compared to individual single therapy was then performed. Consistent with FIG. 8A, and literature [39], Camptothesome-4 or α-PD-L1 alone significantly impeded tumor development, yet combination therapy was far more effective and improved m ice survival (FIG. 11). This effect was enhanced when co-blocking PD-L1 and PD-1 and administering Camptothesome-4 simultaneously, eradicating tumor in 1/6 mice (FIG. 8D). Camptothesome-4 boosted tumor-infiltrated CD8, CTL with enhanced Granzyme B, Perforin, IFN-γ and cleaved caspase-3 (CC-3) production, indicative of CTL-elicited anti-CRC adaptive immunity, which was further bolstered in combination therapies (FIG. 8H). To verify whether combination therapies can achieve similar therapeutic effects in other CRC type, the efficacy in MC38 tumor model was investigated. Consistent with literature, α-PD-L1 elicited noticeable tumor reduction [39]. This effect was more significant when combined with α-PD-1. Strikingly, a single IV Camptothesome-4 administration shrank tumors in 5/6 mice and eliminated tumors in 1/6 mice; co-blocking PD-1 and PD-L1, a single IV injection of Camptothesome-4 eradicated tumors in 5/6 mice with appreciably extended mice survival rate (FIG. 8I 8L); surprisingly, these 5 surviving mice maintained tumor-free after re-challenged with MC38 cells, revealing the development and activation of the memory T cell immunity vital for preventing tumor recurrence (FIG. 8M). To elucidate the mechanism of action for Camptothesome-4-induced CTL antitumor immune response, IFN-γ was systemically knocked down in CT26 tumor-bearing mice, which drastically decreased anti-CRC efficacy and CTL adaptive immunity in combination therapies as manifested by significantly attenuated tumor growth suppression and Granzyme B, Perforin, and CC-3, suggesting an IFN-γ-dependent antitumor immunity (FIG. 8H).

Remotely Loading DOX-Derived IND into Camptothesome-4

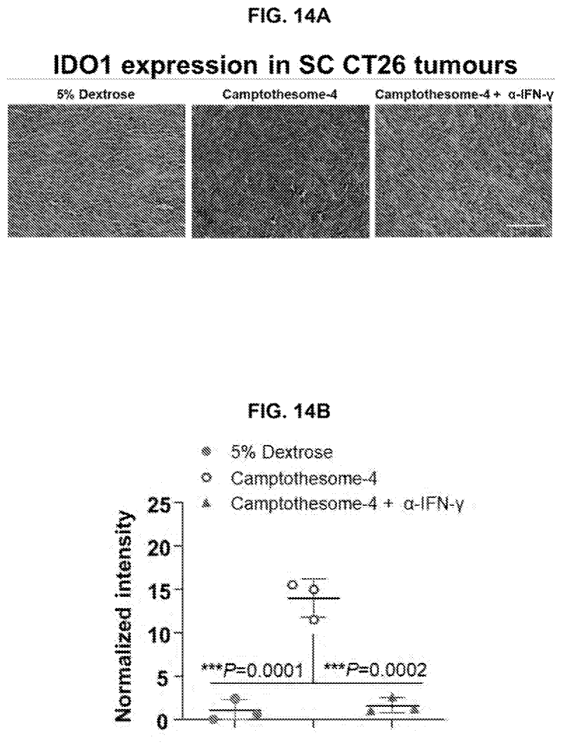

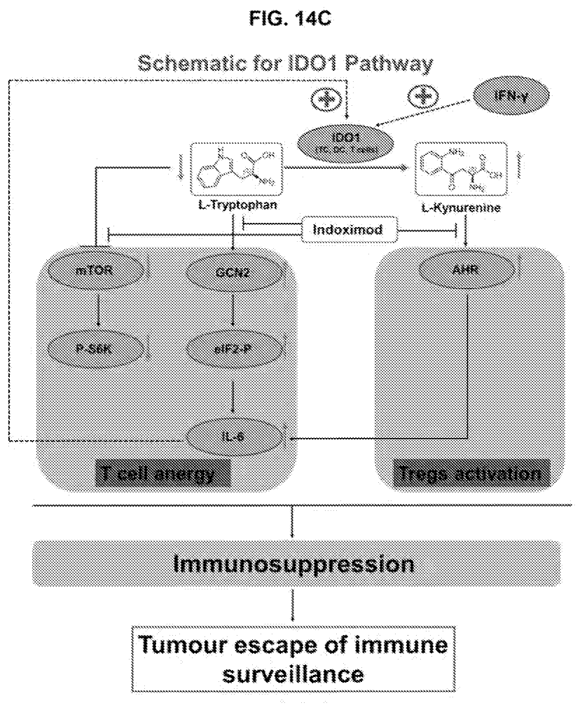

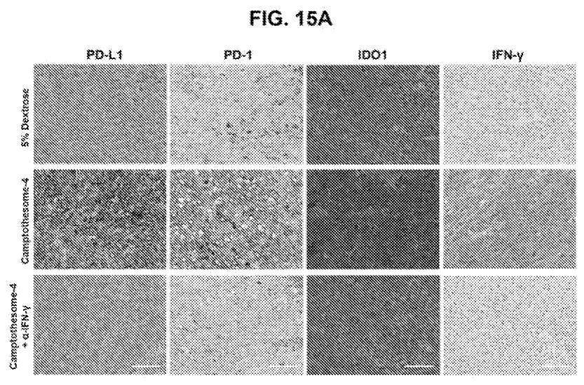

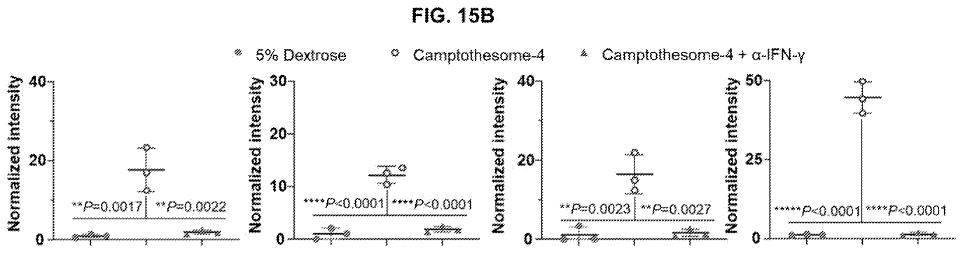

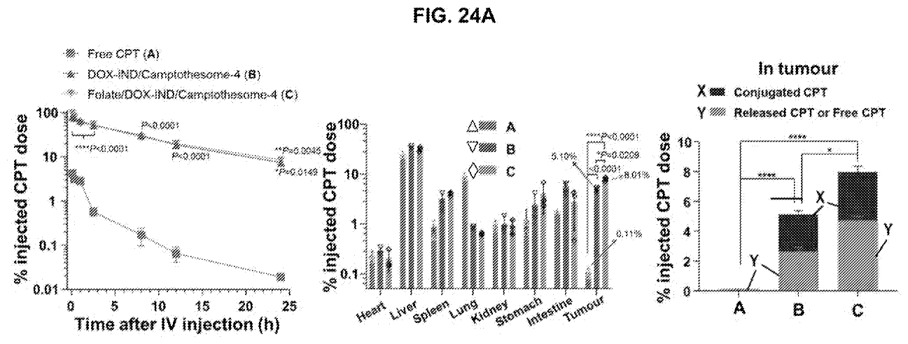

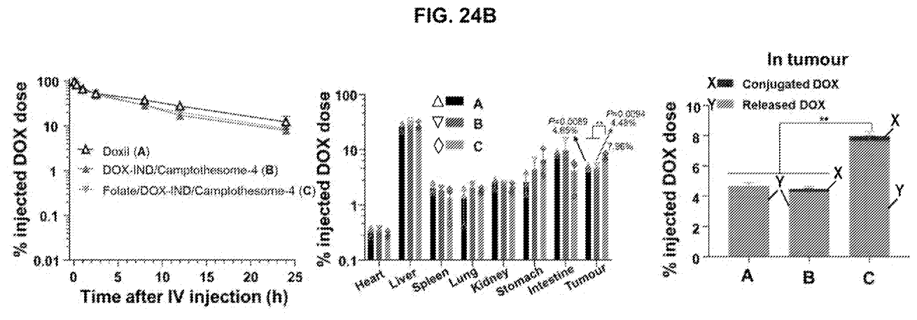

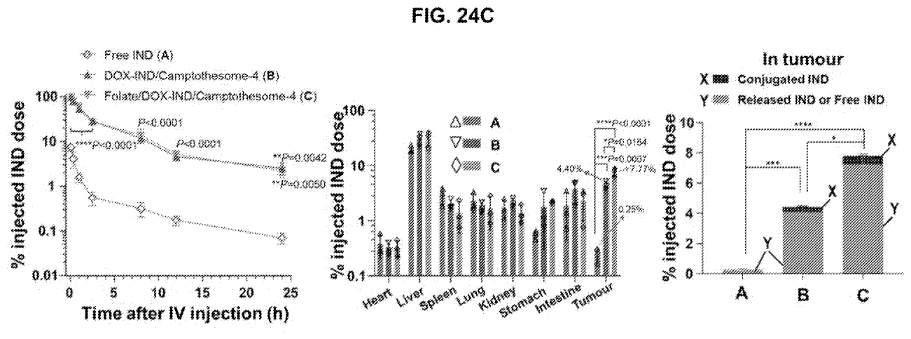

In accordance with previous findings [17, 18], IDO1 is highly expressed in CT26 and MC38 CRC tumors and was further induced by Camptothesome-4-induced INF-γ (FIG. 14-15). Thus, these data prompted testing whether integrating IDO1 inhibitor-IND into Camptothesome-4 enhances therapeutic potential. Direct loading of IND into Camptothesome-4 was found to be limited (<0.5% DLC; FIG. 18; Table 7). To facilitate encapsulation of IND, a DOX-conjugated IND was synthesized (DOX-IND, Scheme 8) with a hydrazone bond on DOX and a disulfide linkage at IND side based on their unique chemical properties. DOX was hypothesized to serve as a transmembrane-enabling agent to bring IND into the interior of Camptothesome-4 since it can readily cross the lipid bilayer. This pH-sensitive hydrazone bond was meticulously devised to be cleaved after crossing the lipid bilayer under acidic environment presented by prefilled protonating agent, (NH₄)₂SO₄, forming drug precipitates inside Camptothesome-4, which avoid drug leakage/escaping. The studies shown herein confirmed that DOX-IND can be imported into Camptothesome 4 where it efficiently broke to release DOX and IND intermediate, yielding drug precipitates in the interior (Scheme 8 and 18B). This DOX-IND-laden Camptothesome-4 exhibited uniform size distribution and accommodated up to 22% of DOX-IND (5.1% IND) that is 10-fold higher than that of IND direct loading (Table 7). Since folate receptor is overexpressed on many tumor cells including CRC [41], it was extrapolated that addition of a folate ligand onto Camptothesome-4 would further enhance the intratumoral uptake and retention of delivered drugs [29]. Incorporation of folate targeting onto the surface of Camptothesome-4 negligibly impacted its size, polydispersity, morphology, and drug loading (FIG. 18B-18C; Table 7). Table 8 shows animal death and weight loss for various doses of DOX-IND/Camptothesome-4. The antitumor efficacy of DOX-IND/Camptothesome-4 with or without folate was tested in large MC38 tumor (300-400 mm³)-bearing mice. Free DOX-IND did not show significant tumor growth inhibition compared to vehicle control, while Camptothesome-4 appreciably controlled tumor development; co-delivering DOX-IND with Camptothesome-4 shrank tumors in 6/6 mice with markedly increased tumor-infiltrated CD8, IFN- while co-administering free DOX-IND marginally improved efficacy (FIG. 16E). Remarkably, folate functionalization allowed DOX-IND/Camptothesome-4 to eradicate large MC38 tumors in 2/6 m ice likely due to the improved intratumoral uptake efficiency (FIG. 24; Table 9). In addition, DOX-IND/Camptothesopme-4 drastically suppressed IDO1 pathway by boosting P-S6K and reducing IL-6 levels, and increased Calreticulin (ICD hallmark), CD8, IFN-γ, Granzyme B, Perforin, and CC-3 expression while simultaneously stunting the Foxp3+ Tregs in tumors; these effects were more prominent with folate tumor targeting (FIG. 16F-16G, 16I and FIG. 20C-20D; Tables 10-11). Of note, Camptothesome-4 elicited better anti-CRC efficacy when co-delivering DOX-IND than co-injecting Doxil® plus IND. This enhancement could arise from improved pharmacokinetics/tumor uptake for IND, and deep tumor penetration (FIG. 7F; FIG. 24). Upon depleting CD8 systemically, the therapeutic efficacy of DOX-IND/Camptothesome-4 was significantly hindered with reduced IFN-γ, Granzyme B, Perforin, and CC-3, strongly indicating the pivotal role CTL-elicited adaptive immunity played in anti-CRC efficacy (FIG. 16E, 16H-16I; FIG. 20). Combining with PD-L1/PD-1 co-blockade, co-delivery of DOX-IND/Camptothesome-4 eliminated ˜400 mm³ tumors in 3/5 mice and extended mice survival prominently; this synergistic combination therapy was IFN-γ-dependent, as neutralizing IFN-γ significantly diminished efficacy (FIG. 16K-16N).

TABLE 7
Physicochemical characterizations of DOX-IND/Camptothesome-4,
Folate/DOX-IND/Camptothesome-4, and IND/Camptothesome-4

| | DOX-IND/ | Folate/DOX-IND/ | IND/ |
|---|---|---|---|
| | Camptothesome-4 | Camptothesome-4 | Camptothesome-4 |
| DOX-IND DLC (%) | 22.0% ± 2.4 | 20.9% ± 3.3 | N/A |
| IND DLC (%) | 5.1% ± 0.9 | 4.9% ± 1.2 | <0.5% |
| DOX DLC (%) | 12.8% ± 1.6 | 12.1% ± 2.1 | N/A |
| DLE (%) | 83.6% ± 4.2 | 80.1% ± 3.9 | 29.3% ± 3.7 |
| DLS Size (nm) | 100.2 ± 4.13 | 101.1 ± 3.24 | 214.1 ± 19.82 |
| Polydispersity | 0.105 ± 0.067 | 0.096 ± 0.059 | 0.231 ± 0.091 |
| Zeta (mV) | −25.8 ± 3.40 | −26.7 ± 2.11 | −20.5 ± 4.59 |

TABLE 8
Death and weight loss for various
doses of DOX-IND/Camptothesome-4

| | CPT/DOX-IND | | |
|---|---|---|---|
| Formulation | Dose (mg/kg) | Animal Death | Weight Loss (%) |
| DOX-IND/ | 15/5 | 0/3 | 1.16 |
| Camptothesome-4 | 20/6.7 | 0/3 | 12.1 |
| | 25/8.3 | 1/3 | N/A |
| | 30/10 | 2/3 | N/A |

TABLE 9
Blood kinetics of CPT

| | | DOX-IND/ | Folate/DOX-IND/ |
|---|---|---|---|
| | Free CPT | Camptothesome-4 | Camptothesome-4 |
| T½ (h) | 0.06 ± 0.01 | 5.60 ± 0.68 | 4.64 ± 0.25 |
| V (μg/(μg/mL)) | 0.77 ± 0.23 | 1.29 ± 0.10 | 1.17 ± 0.02 |
| CL (μg/(μg/mL)/h) | 10.62 ± 1.00 | 0.16 ± 0.01 | 0.18 ± 0.01 |
| AUC0-t (μg/mL*h) | 9.47 ± 0.87 | 2365.19 ± 166.45 | 2225.37 ± 111.96 |
| AUC0-inf (μg/mL*h) | 9.48 ± 0.88 | 2497.07 ± 214.81 | 2289.75 ± 126.47 |
| AUMC (μg/mL*h2) | 8.12 ± 0.38 | 20279.95 ± 4095.06 | 15365.22 ± 1623.18 |
| MRT (h) | 0.86 ± 0.04 | 8.07 ± 0.98 | 6.70 ± 0.35 |
| Vss (μg/(μg/mL)) | 9.16 ± 1.30 | 1.29 ± 0.10 | 1.17 ± 0.02 |

TABLE 10
Blood kinetics of DOX

| | | DOX-IND/ | Folate/DOX-IND/ |
|---|---|---|---|
| | Doxil ® | Camptothesome-4 | Camptothesome-4 |
| T½ (h) | 4.01 ± 0.23 | 3.24 ± 0.99 | 3.03 ± 0.86 |
| V (μg/(μg/mL)) | 1.19 ± 0.01 | 1.12 ± 0.08 | 1.11 ± 0.04 |
| CL (μg/(μg/mL)/h) | 0.13 ± 0.01 | 0.18 ± 0.03 | 0.19 ± 0.03 |
| AUC0-t (μg/mL*h) | 587.89 ± 15.88 | 432.03 ± 57.66 | 436.79 ± 65.96 |
| AUC0-inf (μg/mL*h) | 639.74 ± 22.20 | 443.70 ± 69.18 | 448.19 ± 75.28 |
| AUMC (μg/mL*h2) | 6108.87 ± 428.73 | 2844.68 ± 1118.33 | 2868.49 ± 1037.24 |
| MRT (h) | 9.54 ± 0.34 | 6.26 ± 1.43 | 6.26 ± 1.24 |
| Vss (μg/(μg/mL)) | 1.19 ± 0.01 | 1.12 ± 0.08 | 1.11 ± 0.04 |

TABLE 11
Blood kinetics of IND

| | | DOX-IND/ | Folate/DOX-IND/ |
|---|---|---|---|
| | Free IND | Camptothesome-4 | Camptothesome-4 |
| T½ (h) | 0.08 ± 0.02 | 2.32 ± 0.05 | 2.48 ± 0.08 |
| V (μg/(μg/mL)) | 4.94 ± 0.63 | 1.07 ± 0.04 | 1.06 ± 0.03 |
| CL (μg/(μg/mL)/h) | 12.08 ± 1.17 | 0.53 ± 0.01 | 0.52 ± 0.02 |
| AUC0-t (μg/mL*h) | 5.66 ± 0.53 | 59.64 ± 1.36 | 65.08 ± 2.73 |
| AUC0-inf (μg/mL*h) | 5.67 ± 0.54 | 59.65 ± 1.37 | 65.09 ± 2.74 |
| AUMC (μg/mL*h2) | 2.38 ± 0.71 | 112.51 ± 5.71 | 132.27 ± 11.64 |
| MRT (h) | 0.41 ± 0.09 | 1.88 ± 0.07 | 2.03 ± 0.11 |
| Vss (μg/(μg/mL)) | 4.95 ± 0.63 | 1.07 ± 0.03 | 1.06 ± 0.03 |

### Eliminating Advanced and Metastatic Orthotopic CRC or Melanoma Tumors in Mice

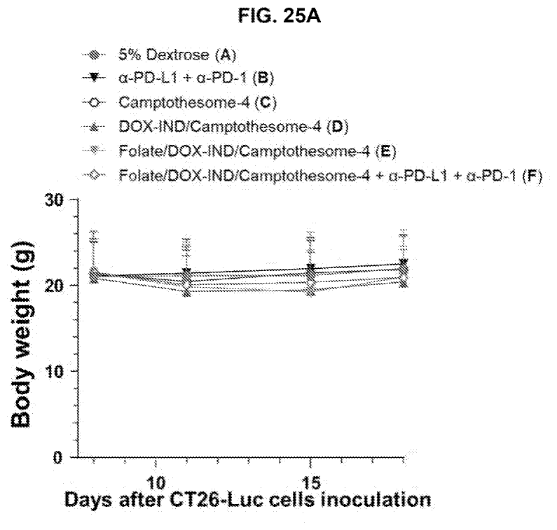

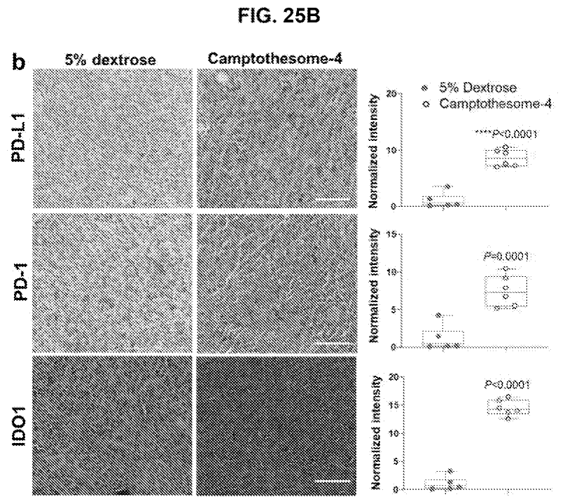

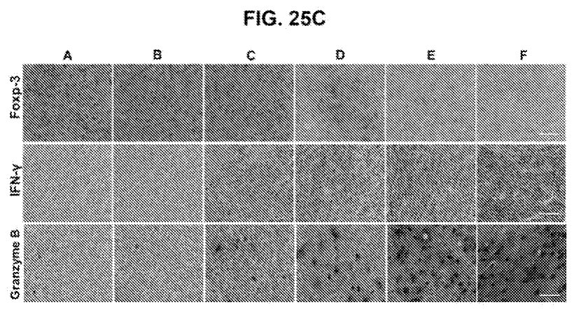

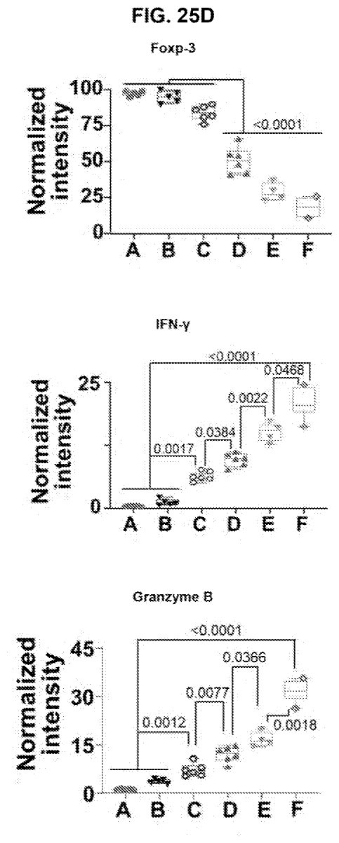

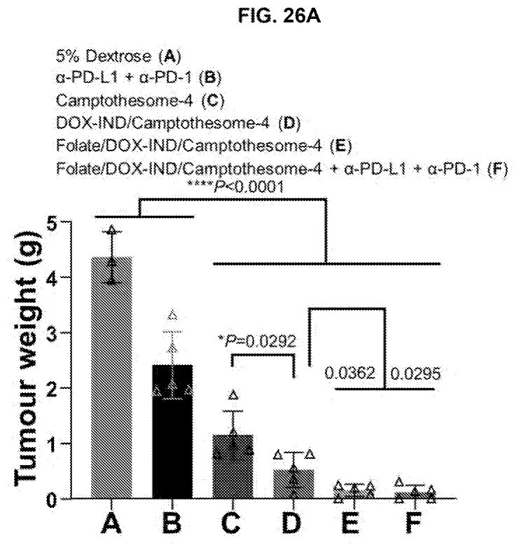

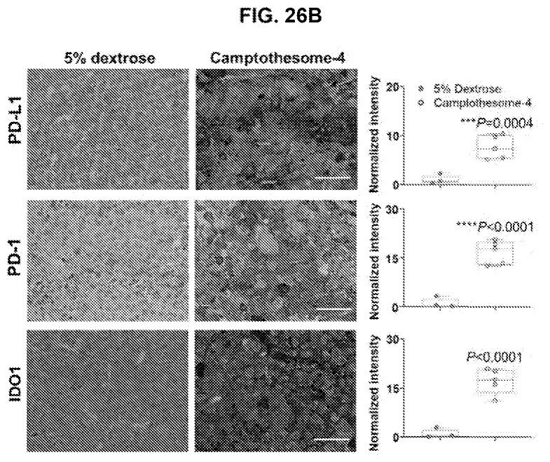

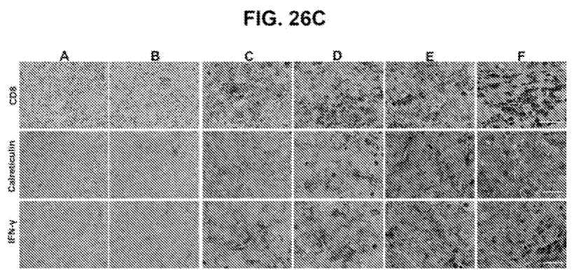

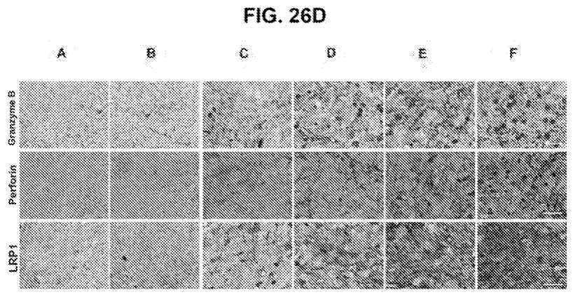

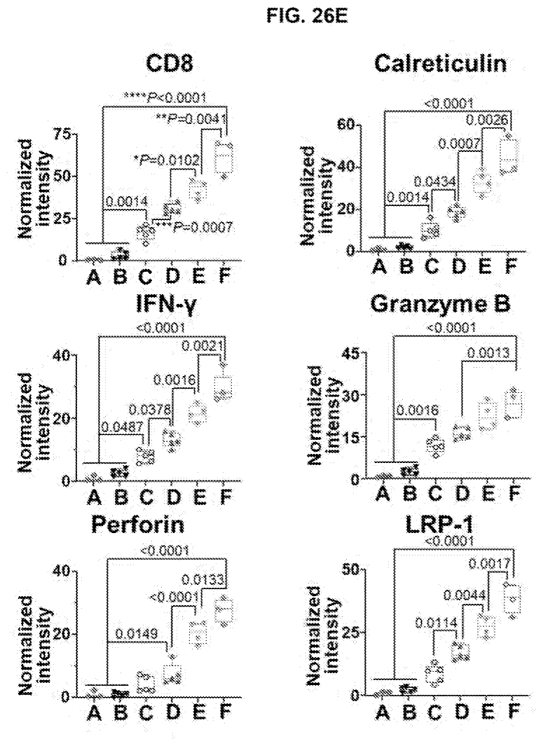

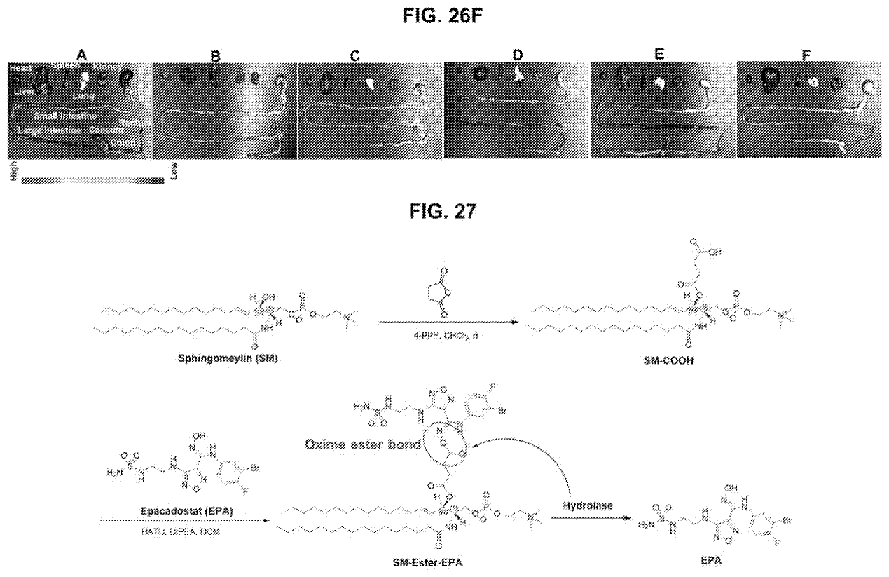

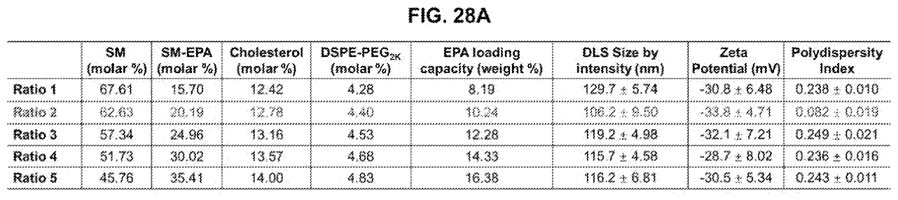

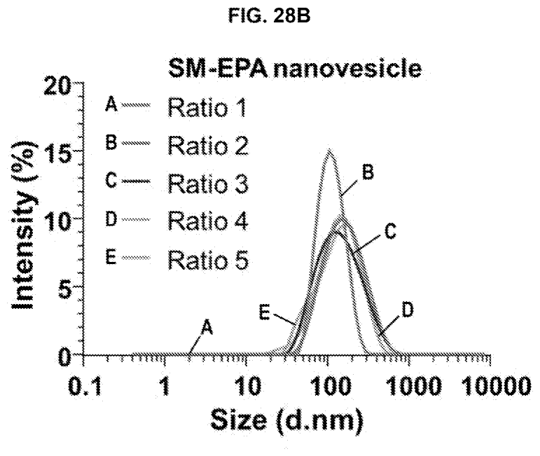

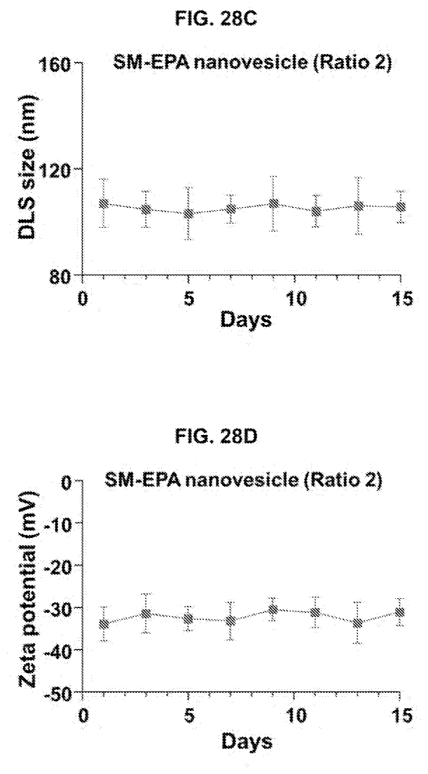

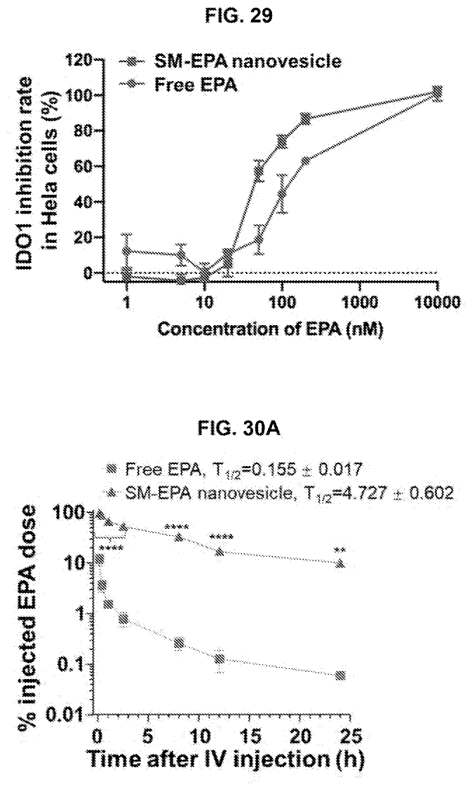

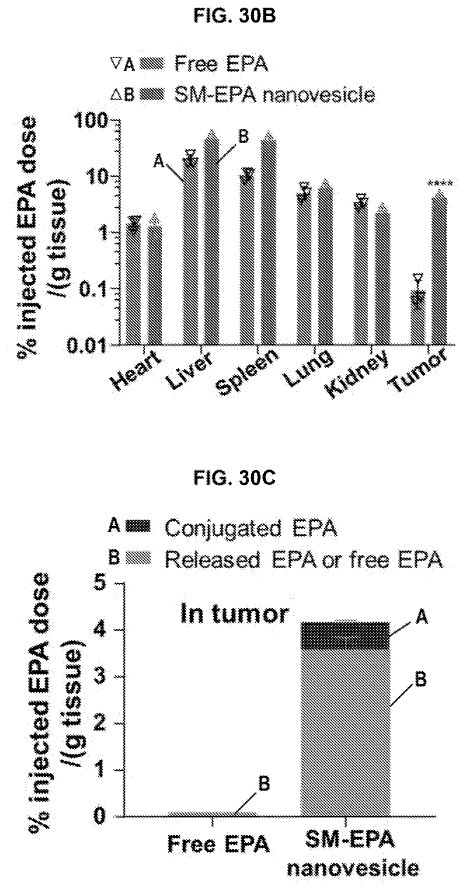

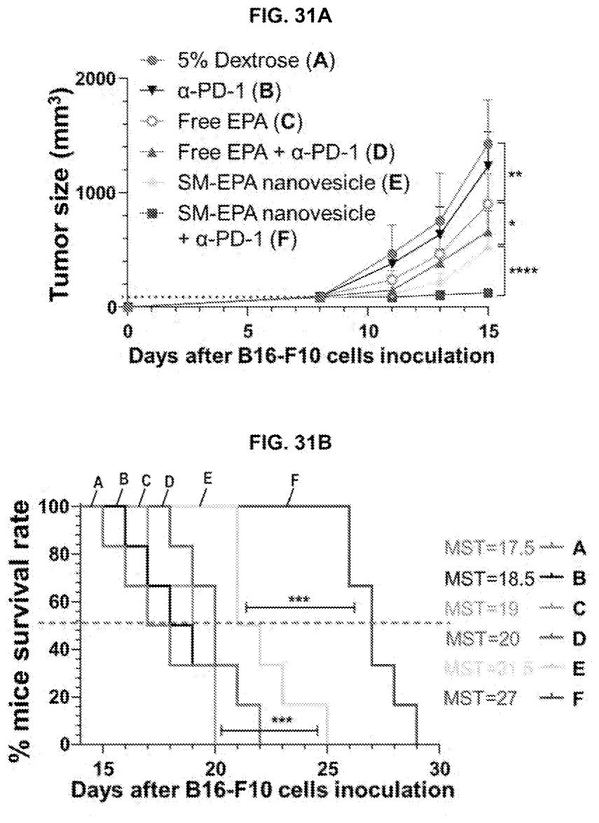

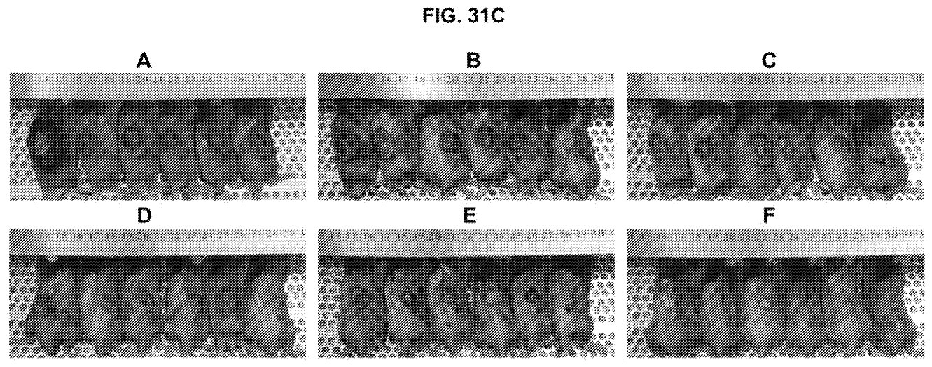

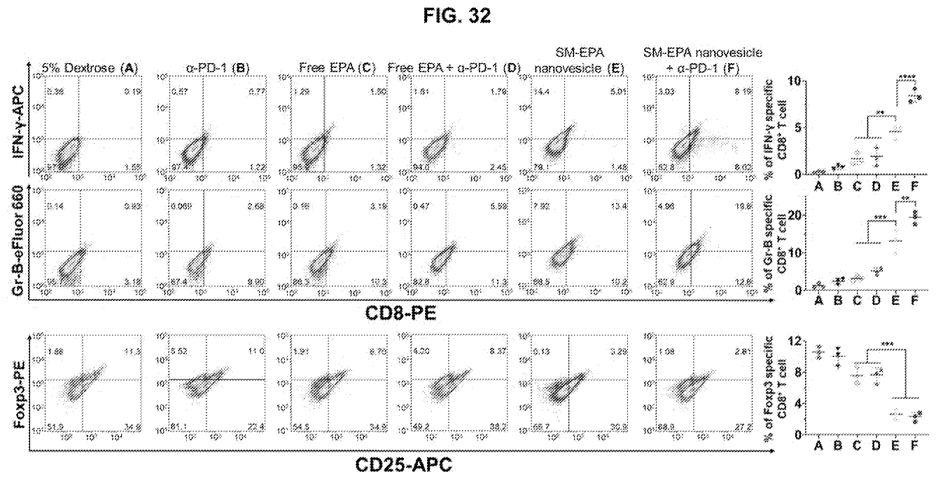

To explore therapeutic potential of the co-delivery Camptothesome-4 in a more profound manner, advanced syngeneic murine models bearing late-stage and metastatic orthotopic CRC or melanoma were established. In metastatic orthotopic CRC mouse model (˜300 mg; FIG. 23), tumors in vehicle control mice grew uncontrollably with one death on day 18 post inoculating CT26-Luc cancer cells into cecum subserosa [42], and metastasized to various internal organs including liver, spleen, kidneys, stomach, intestines, colon and rectum, etc. (FIG. 6A-6F), demonstrating the remarkable aggressiveness and invasiveness of this malignant tumor. α-PD-L1+α-PD-1 therapies had negligible effect in controlling primary tumor and preventing metastasis with one mouse dying on day 18, suggesting the poor responsiveness of this tumor to ICB (FIG. 6A-6F). Camptothesome-4 monotherapy produced a significant tumor reduction and suppressed tumor spread; these effects were markedly enhanced by co-delivering DOX-IND (FIG. 6A-6F). With folate targeting, DOX-IND/Camptothesome-4 further detained tumor growth and eradicated tumors in 40% mice. When combined with PD-L1/PD-1 co-blockade, Folate/DOX-IND/Camptothesome-4 rendered 66.7% mice survived tumor-free with no detectable metastasis (FIG. 6A-6F), boosting antitumor immunity as manifested by dramatically bolstering calreticulin, HMGB-1 (ICD initiators), LRP1, and TLR4 (receptors on dendritic cells for Calreticulin and HMGB-1 uptake, respectively, during ICD) [43, 44], CD8, Perforin, Granzyme B, and CC-3, and proinflammatory cytokines-IL-12 and IFN-γ, while concurrently interfering Tregs development, and inhibiting anti-inflammatory IL-10 in tumors (FIG. 6G; FIG. 25). To confirm that this synergistic antitumor efficacy is applicable to other tumor types, the same combination therapeutic regimen was assessed in mice bearing large and late-stage melanoma (˜400 mm³). Similarly, in mice bearing late-stage melanoma (˜400 mm³), Folate/DOX-IND/Camptothesome-4 eliminated large primary melanoma tumor in 1/5 mice, enhancing CTL anticancer immune response; upon co-blocking PD-L1/PD-1, Folate/DOX-IND/Camptothesome-4 eradicated primary tumors in 40% mice with complete metastasis remission (FIG. 6H-6K; FIG. 26).

### CONCLUSION

Among FDA-approved cancer nanomedicines, except Abraxane® (album in-bound paclitaxel), all are liposome-based because of the biocompatibility/biodegradability, and favorable in vivo stability. However, current liposomal platforms only work for hydrophilic drugs, as incorporation of hydrophobic therapeutics jeopardizes the integrity of the lipid bilayer. Inspired by liposome's clinical success and suitability, a novel and versatile Camptothesome platform was developed where CPT is securely anchored in the lipid bilayer and where CPT is covalently conjugated to the liposome's backbone component-SM. This SM-conjugation approach has the potential to rescue CPT and solves the limitations associated with a significant portion (50-60%) of poorly soluble small molecule drugs containing functional groups [45]. Additionally, the amide linkage in SM increases intermolecular hydrogen bonding and formulation stability in vivo compared to the ester bonds of diacyl chains in other phospholipids under physiological conditions [46]. SM serves as the backbone component in FDA-approved Margibo®, a liposomal vincristine sulfate; Cholesterol and DSPE-PEG2K are used in many FDA-approved liposomal nanotherapeutics (e.g., Doxil®, Onivyde®). In addition, the manufacturing procedure of Camptothesome is facile, standardized, well-established, and identical to traditional liposomal nanoformulations. Thus, the Camptothesome nanoplatform boasts promising clinical relevance and could be potentially translated into clinic, considering its exceptional safety profiles, improved pharmacokinetics/tumor accumulation, and remarkable antitumor efficacy by itself or in combination with PD-L1/PD-1 co-blockade therapy, which eradicated established MC38 tumors in 83.3% mice and activated the memory T cell immunity for tumor recurrence prevention (FIG. 6-8).

Furthermore, Camptothesome enables co-delivery of IND using DOX as a transmembrane-enabling agent. Sensitive to the acidic pH, the hydrazone bond of DOX-IND breaks inside Camptothesome, releasing free DOX. Without DOX conjugation, parent IND would be liberated more efficiently from IND intermediate due to less steric hindrance under high glutathione/hydrolase levels in tumor tissues/cells [22, 46, 47]. Moreover, the intraliposomal acidic environment helps stabilize the lactone ring of CPT.

The DOX-enabled transmembrane transportation technology opens a new venue for temporal-spatial controlled co-delivery of various therapeutics not loadable by existing liposomal platforms. The synergistic combination chemo-immunotherapy could be attributed to: (1) improved pharmacokinetics, enhanced tumor accumulation/retention, and efficient extravasation/tumor penetration, as well as controlled/sustained intratumoral drug release; (2) Camptothesome-4 elicited first round of immune responses by augmenting CTL killing of tumor cells and PD-L1/PD-1 expression to potentiate PD-L1/PD-1 blockade; (3) subsequent IND and DOX release from Camptothesome-4 further enhanced and/or sustained the magnitude of antitumor immunity through overcoming IDO1-induced immunosuppression (e.g., stunted Tregs) and concurrently eliciting ICD (e.g., stimulated calreticulin/LRP1 and HMGB-1/TLR4) (FIG. 8C, H; 16J; 22F).

This is the first nanotherapeutic platform developed using SM-conjugated drug and first DOX-enabled transmembrane transporting technology reported, both of which are generalizable to various therapeutics. Given that (1) SM, Cholesterol, and DSPE-PEG2K are used in many FDA-approved liposomal nanotherapeutics (e.g., Marqibo®, Doxil®, Onivyde®); (2) the manufacturing procedure of Camptothesome or co-delivery of Camptothesome is facile, standardized, and well-established as similar to traditional liposomal nanoformulations; (3) the remarkable efficacy achieved against both early-stage and clinically-difficult-to-treat late-stage metastatic orthotopic tumors; and (4) IDO1's expression in diverse cancer cells, our robust and multi-pronged Camptothesome immunochemotherapy framework boasts promising clinical relevance and could potentially revolutionize cancer treatment paradigms.

The Camptothesome platform described herein has the potential to significantly improve cancer patient responses. While the foundational framework presented herein partially cured tumors by a single IV administration of co-delivery nanotherapeutics with or without ICB, higher tumor remission rate is feasible through adjusting dosage and/or dosing frequency premised on the MTD and overall antitumor immunity, or further combining with other therapeutic modalities (e.g., cytokines, TLR agonists, and photodynamic therapy) that induce complementary immune responses. This is the first nanotherapeutic derived by SM-conjugation and DOX-enabled transmembrane transporting technology reported, both of which are generalizable to a wide variety of therapeutics; given IDO1's expression in diverse cancer cells, the multi-pronged Camptothesome framework can potentially revolutionize cancer treatment paradigms.

### REFERENCES

1. Haslam and Prasad, “Estimation of the Percentage of US Patients with Cancer Who Are Eligible for and Respond to Checkpoint Inhibitor Immunotherapy Drugs,” JAMA network open 2: e192535 (2019).
2. Catenacci et al., “Keeping Checkpoint Inhibitors in Check,” JAMA network open 2: e192546 (2019).
3. Overman et al., “Where We Stand with Immunotherapy in Colorectal Cancer: Deficient Mismatch Repair, Proficient Mismatch Repair, and Toxicity Management,” Am. Soc. Clin. Oncol. Educ. Book 38: 239-247 (2018).
4. Huyghe et al., “Immunotherapy with immune checkpoint inhibitors in colorectal cancer: what is the future beyond deficient mismatch-repair tumours?” Gastroenterol. Rep. 8: 11-24 (2020).
5. Galon and Bruni, “Approaches to treat immune hot, altered and cold tumours with combination immunotherapies,” Nat. Rev. Drug. Discov. 18: 197-218 (2019).
6. Song et al., “Synergistic and low adverse effect cancer immunotherapy by immunogenic chemotherapy and locally expressed PD-L1 trap,” Nat. Commun. 9: 2237 (2018).
7 Paz-Ares et al., “Pembrolizumab plus Chemotherapy for Squamous Non-Small-Cell Lung Cancer,” N. Engl. J. Med. 379: 2040-2051 (2018).
8. Boland et al., “A phase Ib/II study of cetuximab and pembrolizumab in RAS-wt mCRC,” J. Clin. Oncol. 36 (2018).
9. Ebert et al., “MAP Kinase Inhibition Promotes T Cell and Anti-tumor Activity in Combination with PD-L1 Checkpoint Blockade,” Immunity 44: 609-621 (2016).
10. Andtbacka et al., “Talimogene Laherparepvec Improves Durable Response Rate in Patients with Advanced Melanoma,” J. Clin. Oncol. 33: 2780-2798 (2015).
11. Chen et al., “Host immune response to anti-cancer cam ptothecin conjugated cyclodextrin-based polymers.” J. Biomed. Sci. 26: 85 (2019).
12. Lu et al., “The self-assembling camptothecin-tocopherol prodrug: An effective approach for formulating cam ptothecin,” Biomaterials 62: 176-187 (2015).
13. Dosset et al., “PD-1/PD-L1 pathway: an adaptive immune resistance mechanism to immunogenic chemotherapy in colorectal cancer,” Oncoimmunology 7, e1433981 (2018).
14. Prendergast et al., “Indoleamine 2,3-dioxygenase pathways of pathogenic inflammation and immune escape in cancer,” Cancer Immunol. Immunother. 63: 721-735 (2014).
15. Hou et al., “Inhibition of indoleamine 2,3-dioxygenase in dendritic cells by stereoisomers of 1-methyl-tryptophan correlates with antitumor responses,” Cancer Res. 67: 792-801 (2007).
16. Lob et al., “Inhibitors of indoleamine-2,3-dioxygenase for cancer therapy: can we see the wood for the trees?” Nat. Rev. Cancer 9: 445-452 (2009).
17. Phan et al., “Salmonella-mediated therapy targeting indoleamine 2, 3-dioxygenase 1 (IDO) activates innate immunity and mitigates colorectal cancer growth,” Cancer Gene Ther. 27: 235-245 (2020).
18. Gomes et al., “Characterization of the Selective Indoleamine 2,3-Dioxygenase-1 (IDO1) Catalytic Inhibitor E0S200271/PF-06840003 Supports IDO1 as a Critical Resistance Mechanism to PD-(L)1 Blockade Therapy,” Mol. Cancer Ther. 17: 2530-2542 (2018).
19. Metz et al., “IDO inhibits a tryptophan sufficiency signal that stimulates mTOR: A novel IDO effector pathway targeted by D-1-methyl-tryptophan,” Oncoimmunology 1: 1460-1468 (2012).
20. Kuai et al., “Elimination of established tumors with nanodisc-based combination chemoimmunotherapy,” Science Adv. 4: eaao1736 (2018).
21. Muller et al., “Inhibition of indoleamine 2,3-dioxygenase, an immunoregulatory target of the cancer suppression gene Bin1, potentiates cancer chemotherapy,” Nat. Med. 11: 312-319 (2005).
22. Kroemer et al., “Immunogenic cell death in cancer therapy,” Ann. Rev. Immunol. 31: 51-72 (2013).
23. Gamcsik et al., “Glutathione levels in human tumors.” Biomarkers 17: 671-691 (2012).
24. Zhong et al., “Cathepsin B-cleavable doxorubicin prodrugs for targeted cancer therap,”. Int. J. Oncol. 42: 373-383 (2013).
25. Shaw and Chou, “Elevation of intracellular glutathione content associated with mitogenic stimulation of quiescent fibroblasts,” J. Cell Physiol. 129: 193-198 (1986).
26. Jessani et al., “Enzyme activity profiles of the secreted and membrane proteome that depict cancer cell invasiveness,” Proc. Natl. Acad. Sci. USA. 99: 10335-10340 (2002).
27. Bucher, “Ester Pyrolysis of Carbonates: Bis(benzene hydrate) Carbonate as Potential Precursor for Monomeric Carbonic Acid,” Eur. J. Org. Chem. 1070-1075 (2010).
28. Lu et al., “PEG-derivatized embelin as a nanomicellar carrier for delivery of paclitaxel to breast and prostate cancers,” Biomaterials 34: 1591-1600 (2013).
29. Lu et al., “Targeted delivery of Doxorubicin by folic acid-decorated dual functional nanocarrier,” Mol. Pharm. 11: 4164-4178 (2014).
30. Lu et al., “An improved D-alpha-tocopherol-based nanocarrier for targeted delivery of doxorubicin with reversal of multidrug resistance,” J. Contr. Rel. 196, 272-286 (2014).
31. Rao et al., “Bouchardatine analogue alleviates non-alcoholic hepatic fatty liver disease/non-alcoholic steatohepatitis in high-fat fed mice by inhibiting ATP synthase activity,” Br. J. Pharmacol. 176, 2877-2893 (2019).
32. Konstantopoulos et al., “Metabolic effects of Crocus sativus and protective action against non-alcoholic fatty liver disease in diabetic rats,” Biomed Rep. 6: 513-518 (2017).
33. Abdel-Salam et al., “Effect of Cannabis sativa on oxidative stress and organ damage after systemic endotoxin administration in mice,” Comp. Clin. Pathol. 23: 1069-1085 (2013).
34. Bhattacharyya et al., “A paclitaxel-loaded recombinant polypeptide nanoparticle outperforms Abraxane in multiple murine cancer models,” Nat. Commun. 6: 7939 (2015).
35. Gabizon et al., “Dose dependency of pharmacokinetics and therapeutic efficacy of pegylated liposomal doxorubicin (DOXIL) in murine models,” J. Drug Target. 10: 539-548 (2002).
36. Soundararajan et al., “[(186)Re]Liposomal doxorubicin (Doxil): in vitro stability, pharmacokinetics, imaging and biodistribution in a head and neck squamous cell carcinoma xenograft model,” Nucl. Med. Biol. 36: 515-524 (2009).
37. Junttila et al., “Antitumor efficacy of a bispecific antibody that targets HER2 and activates T cells,” Cancer Res. 74: 5561-5571 (2014).
38. Clemente-Casares et al., “Expanding antigen-specific regulatory networks to treat autoimmunity,” Nature 530: 434-440 (2016).
39. Song et al., “Trapping of Lipopolysaccharide to Promote Immunotherapy against Colorectal Cancer and Attenuate Liver Metastasis,” Adv. Mater. 30: e1805007 (2018).
40. Finisguerra et al., “MET is required for the recruitment of anti-tumoural neutrophils.” Nature 522: 349-353 (2015).
41. Low et al., “Discovery and development of folic-acid-based receptor targeting for imaging and therapy of cancer and inflammatory diseases,” Acc. Chem. Res. 41: 120-129 (2008).
42. Zhang et al. “Development and characterization of a reliable mouse model of colorectal cancer metastasis to the liver,” Clin. Exp. Metastasis 30: 903-918 (2013).
43. Lu et al., “Nano-enabled pancreas cancer immunotherapy using immunogenic cell death and reversing immunosuppression,” Nat. Commun. 8: 1811 (2017).
44. Lu et al., “Breast Cancer Chemo-immunotherapy through Liposomal Delivery of an Immunogenic Cell Death Stimulus Plus Interference in the IDO-1 Pathway,” ACS nano 12: 11041-11061 (2018).
45. Slotte, “The importance of hydrogen bonding in sphingomyelin's membrane interactions with co-lipids,” Biochem. Biophys. Acta 1858: 304-310 (2016).
46. Narveka ret al., “Nanocarrier for poorly water-soluble anticancer drugs-barriers of translation and solutions,” AAPS PharmSciTech 15: 822-833 (2014).
47. Fox et al., “Synthesis and in vivo antitumor efficacy of PEGylated poly(I-lysine) dendrimer-cam ptothecin conjugates,” Mol. Pharm. 6: 1562-1572 (2009).
48. Zhang et al., “PKSolver: An add-in program for pharmacokinetic and pharmacodynamic data analysis in Microsoft Excel,” Comp. Meth. Prog. Biomed. 99: 306-314 (2010).
49. Goodwin et al., “Local and transient gene expression primes the liver to resist cancer metastasis,” Sci. Transl. Med. 8: 364ra153 (2016).
50. Litzenburger et al., “Constitutive IDO expression in human cancer is sustained by an autocrine signaling loop involving IL-6, STAT3 and the AHR,” Oncotarget 5: 1038-1051 (2014).
51. Kim et al., “The self-assembly of anticancer cam ptothecin-dipeptide nanotubes: a minimalistic and high drug loading approach to increased efficacy,” Chem. Eur. J. 21: 101-105 (2015).
52. Zhang et al., “Transformative Nanomedicine of an Amphiphilic Camptothecin Prodrug for Long Circulation and High Tumor Uptake in Cancer Therapy,” ACS nano 11: 8838-8848 (2017).
53. Zhao et al., “20-O-acylcamptothecin derivatives: evidence for lactone stabilization,” J. Org. Chem. 65: 4601-4606 (2000).
54. Lau et al., “Lactone Stabilization is Not a Necessary Feature for Antibody Conjugates of Camptothecins,” Mol. Pharmac. 15: 4063-4072 (2018).
55. Crowe and Yue, “Semi-quantitative Determination of Protein Expression using Immunohistochemistry Staining and Analysis: An Integrated Protocol,” Bio-protocol 9: (2019).

1. A sphingomyelin-drug conjugate comprising Formula (I): 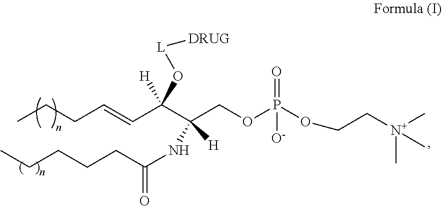
wherein each n is independently 5 to 20;
L is a linker moiety; and
Drug is an anti-cancer drug.

2. The sphingomyelin-drug conjugate of claim 1, comprising Formula (II) 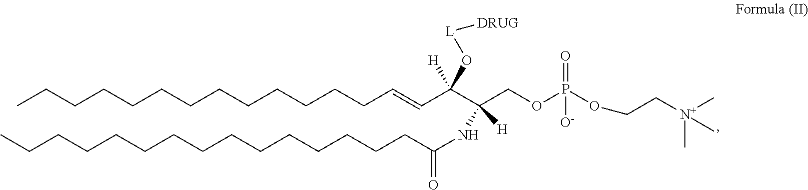
wherein L is a linker moiety; and
Drug is an anti-cancer drug.

3. The sphingomyelin-drug conjugate of claim 1, wherein the anti-cancer drug is hydrophilic or hydrophobic.

4. The sphingomyelin-drug conjugate of claim 1, wherein the anti-cancer drug is selected from the group consisting of: camptothecin (CPT), paclitaxel, docetaxel, ADU-S100, amrubicin, 5-aminolevulinic acid, AZD4635, BMS-1001, BMS-1166, BMS-200, BMS-202, BMS-242, BMS-242, bortezomib, CA170, cabazitaxel, cabozantinib, canertinib, capecitabine, carboplatin, ceritinib, chlorin e6, cisplatin, dabrafenib, dacarbazine, darolutamide, daunorubicin, degarelix, digoxin, doxorubicin, epacadostat, epirubicin, eribulin, esorubicin, etoposide, fingolimod, 5-fluorouracil, galanthamine, gemcitabine, idarubicin, imatinib, imiquimod, indoximod, irinotecan, ixabepilone, lenvatinib, memantine, methotrexate, mitoxantrone, NIR178, NLG919, oxaliplatin, pazopanib, pemetrexed, preladenant, protoporphyrin IX (PPIX), pyropheophorbide-A (PPA), septacidin, SN-38, sorafenib, streptozocin, sunitinib, temozolomide, tipiracil, TPI-287, trifluridine, vadimezan, vemurafenib, vinblastine, vincristine, vinorelbine, vipadenant, vorinostat, and combinations thereof.

5. The sphingomyelin-drug conjugate of claim 1, wherein the sphingomyelin-drug conjugate comprises Formula (III)-(VI): 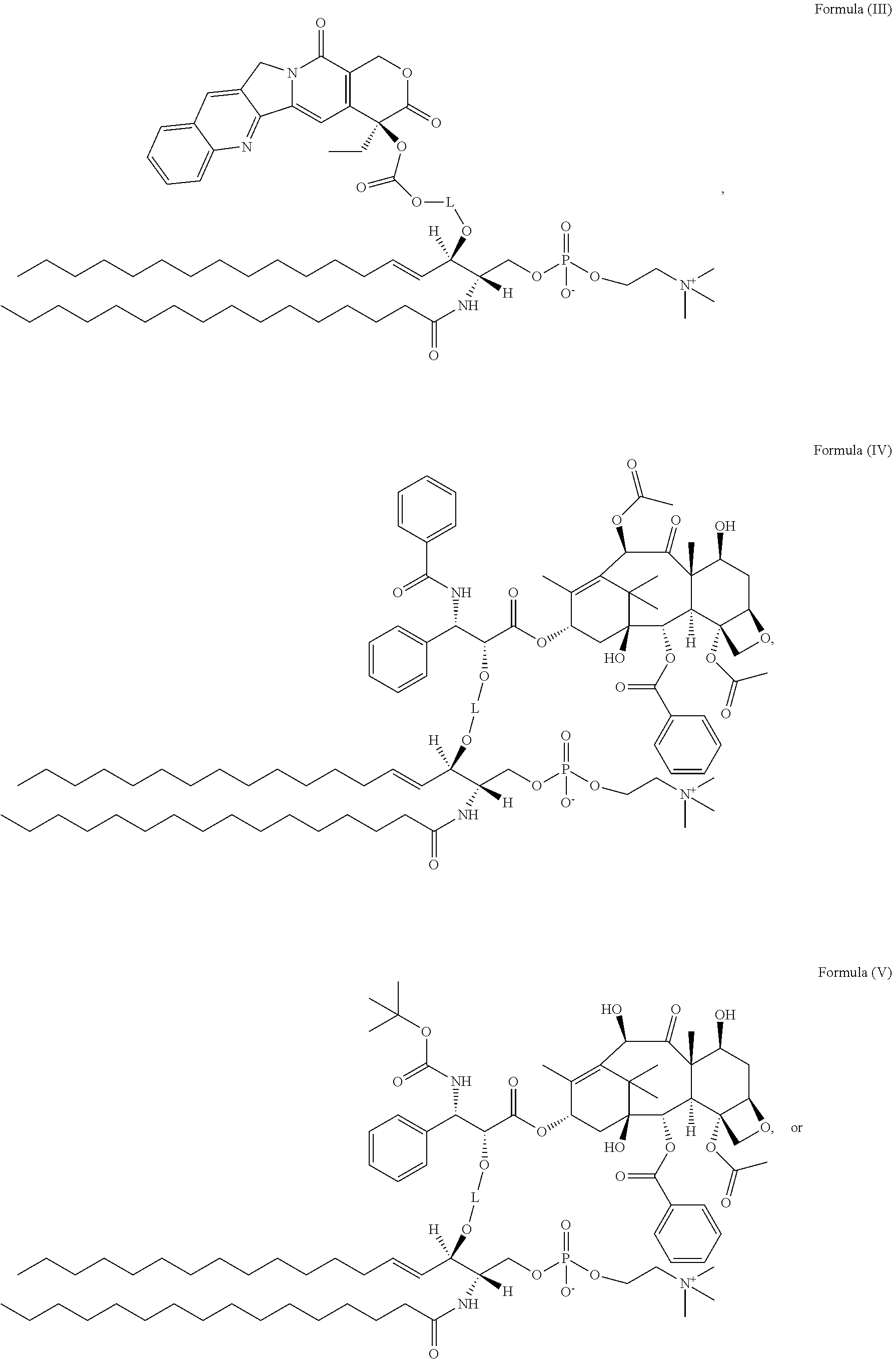 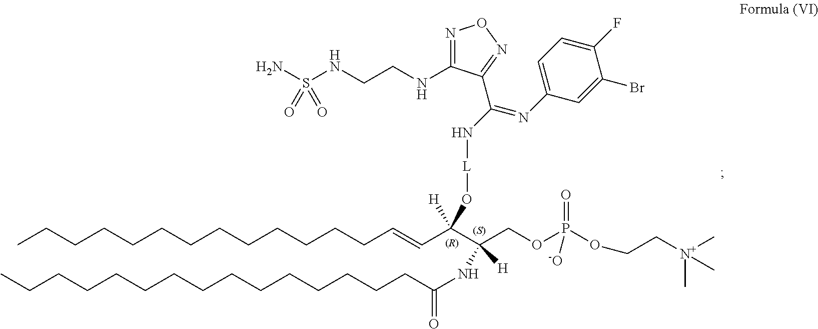
wherein L is a linker moiety.

6. The sphingomyelin-drug conjugate of claim 1, wherein L is selected from: 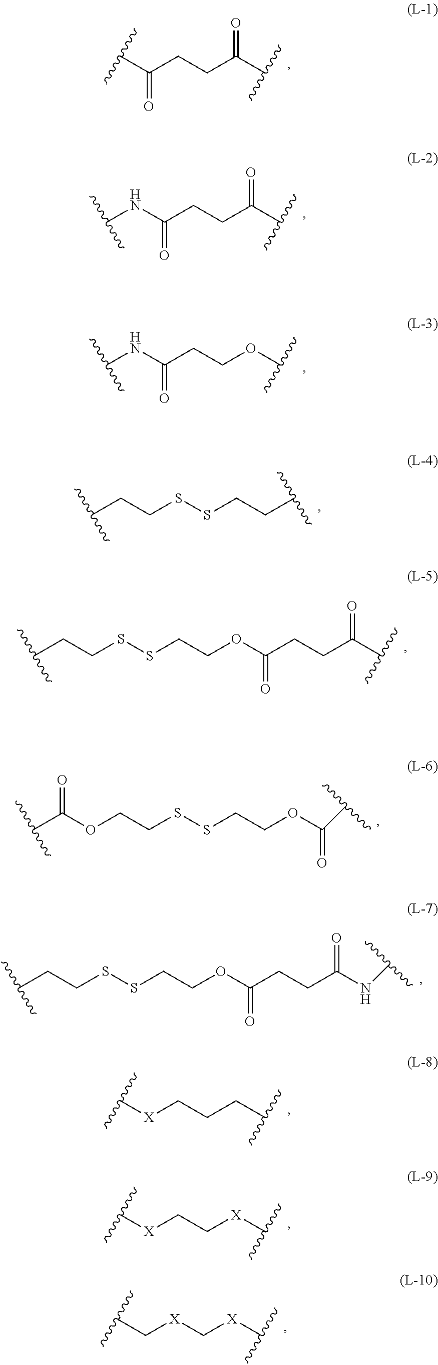 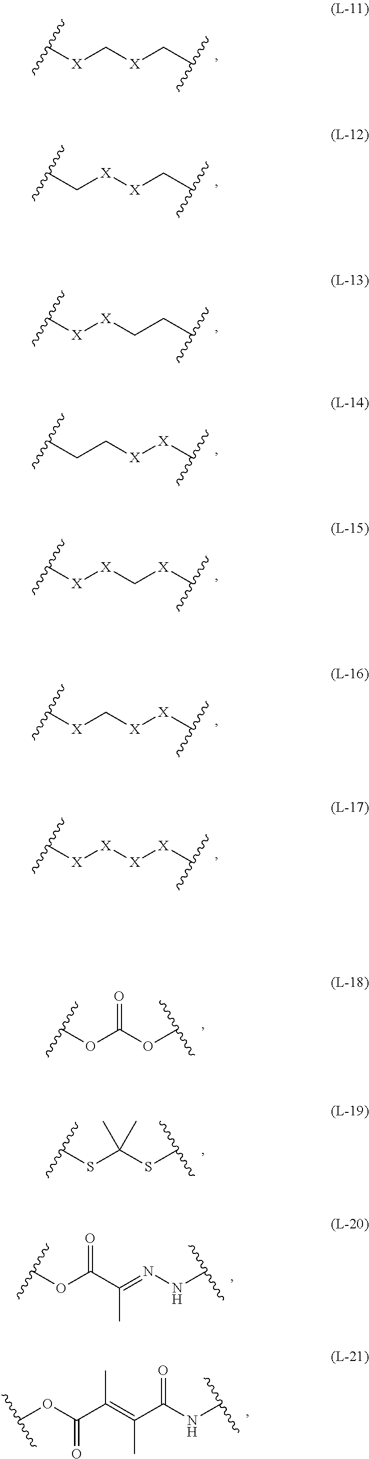 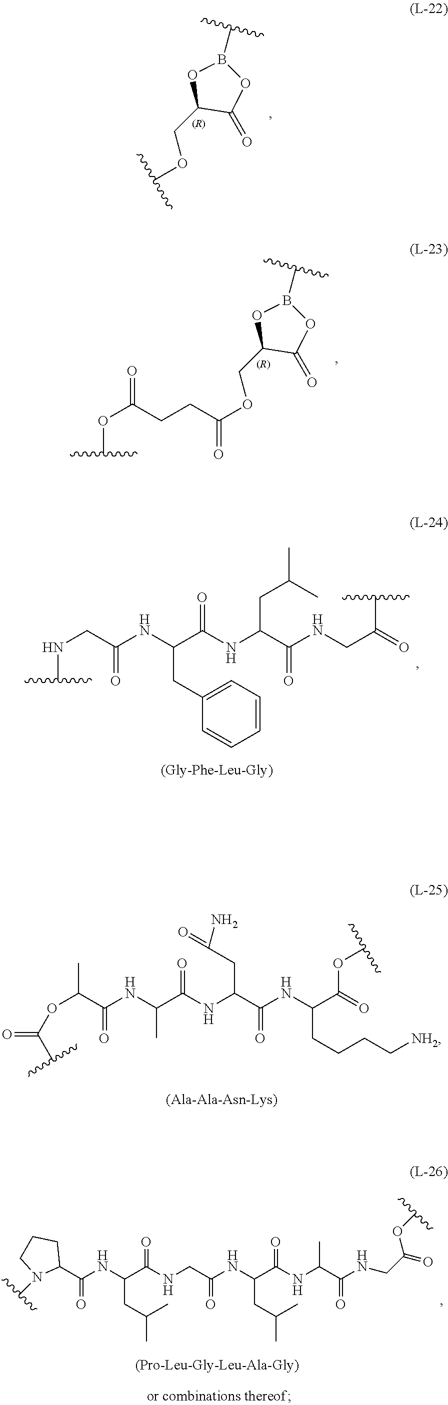
wherein X is independently, O, S, —NH, or —CO.

7. The sphingomyelin-drug conjugate of claim 1, wherein the sphingomyelin-drug conjugate is:

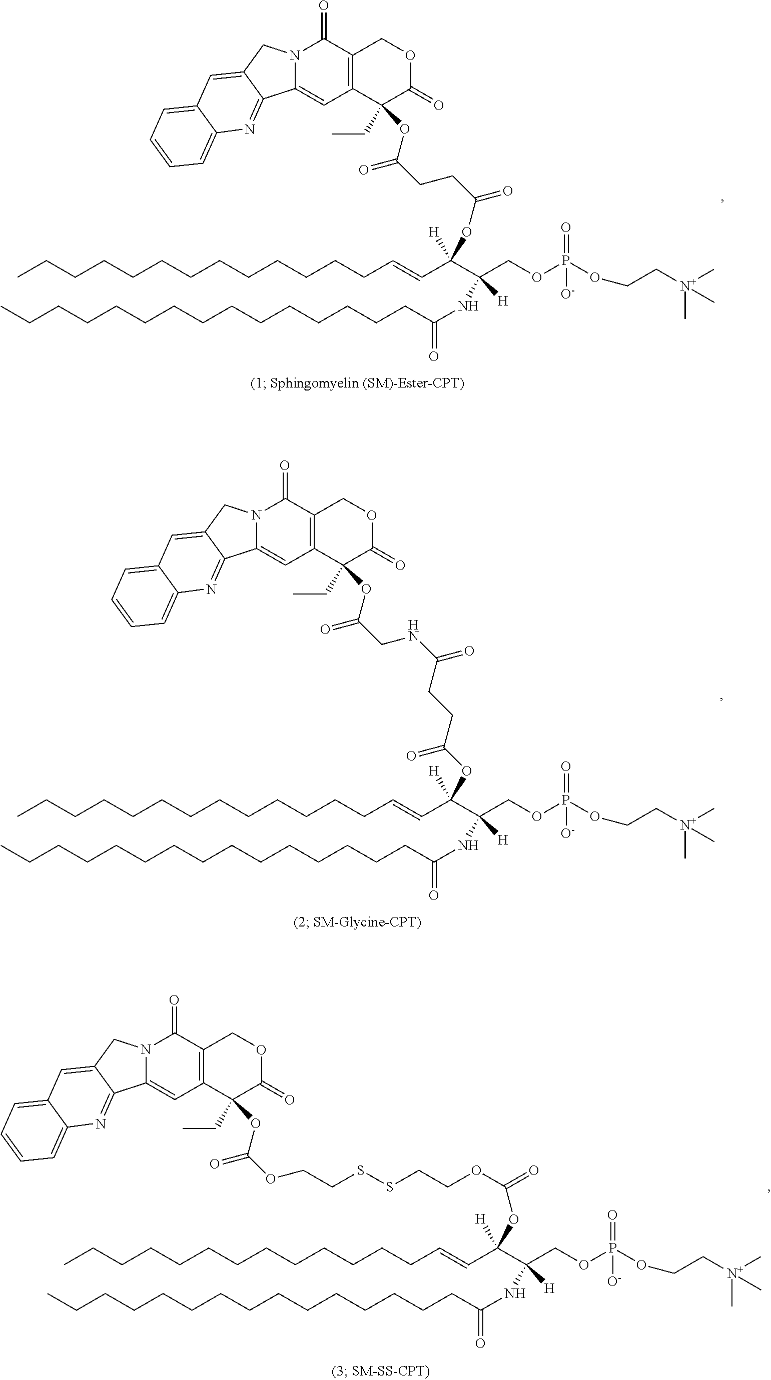

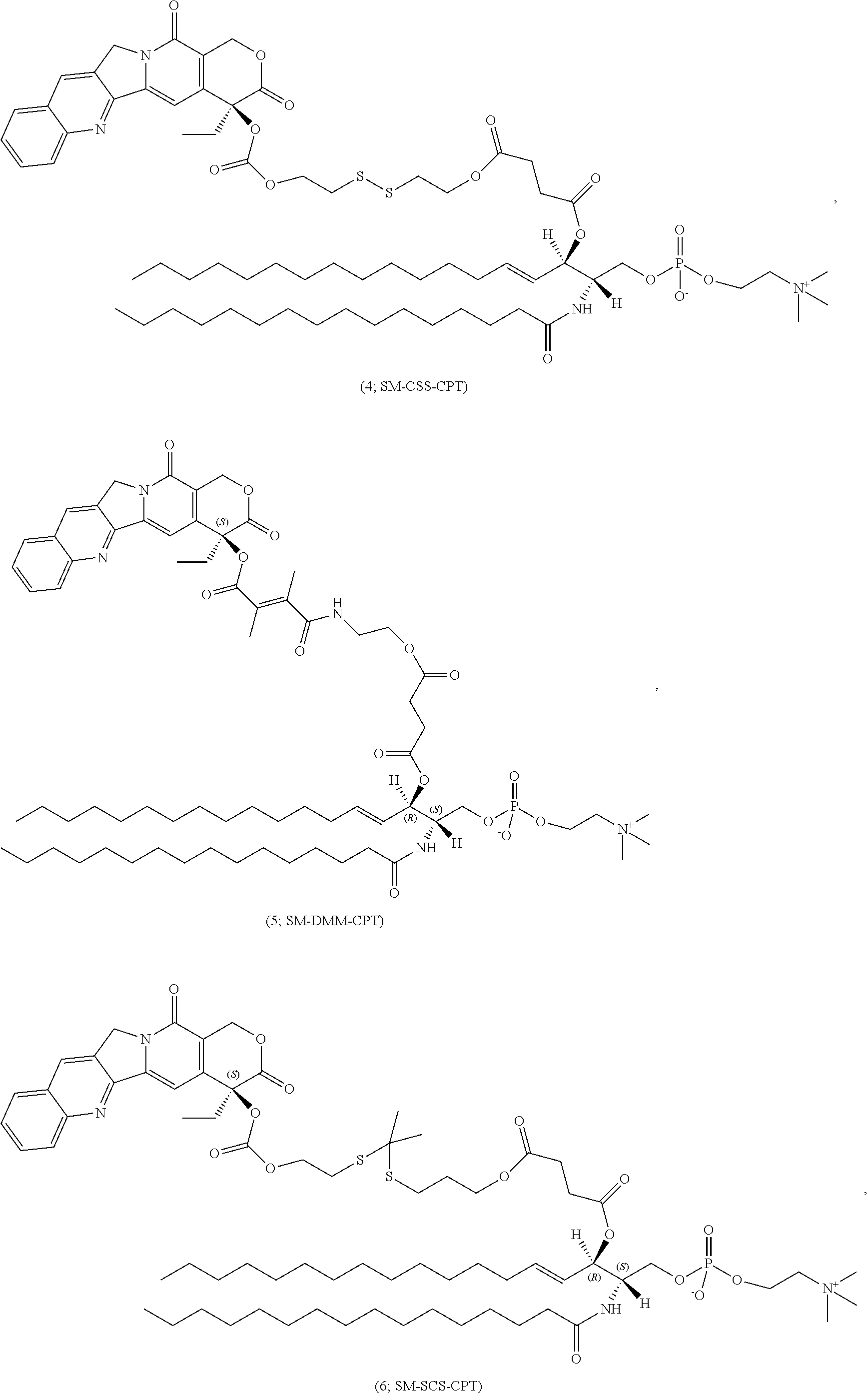

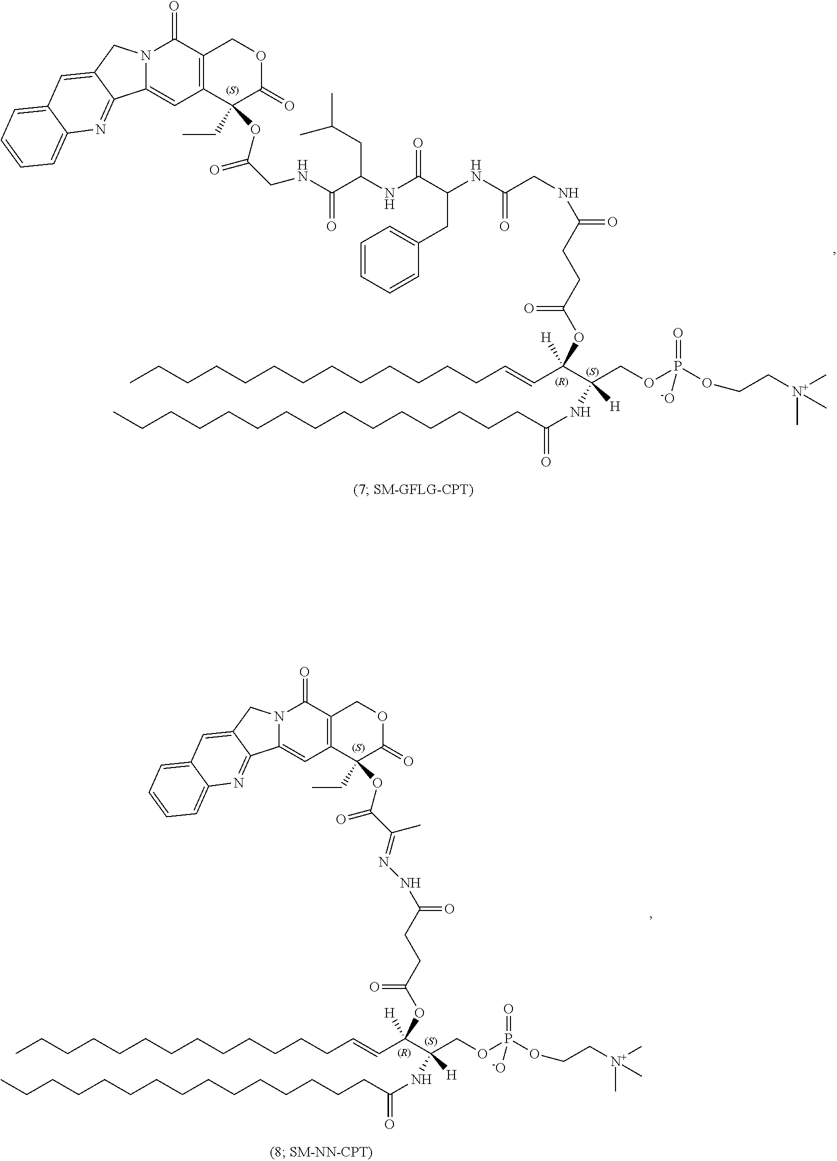

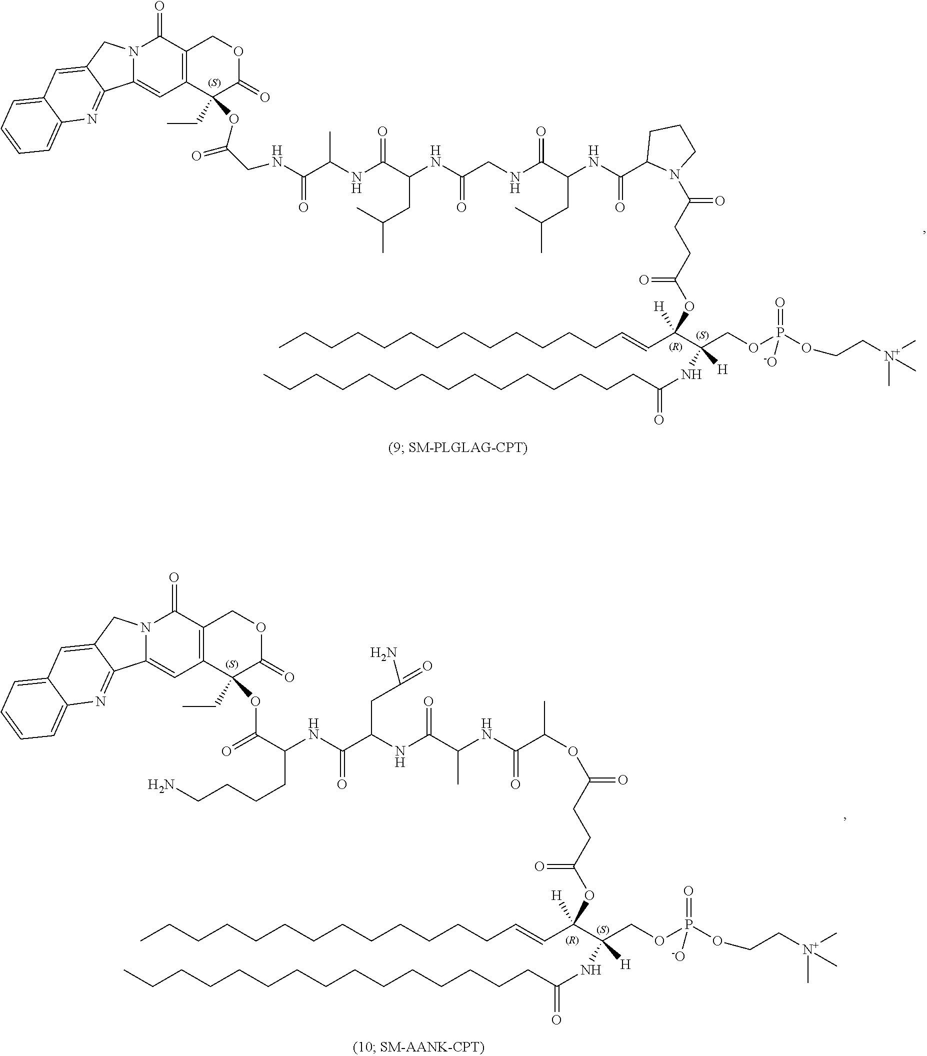

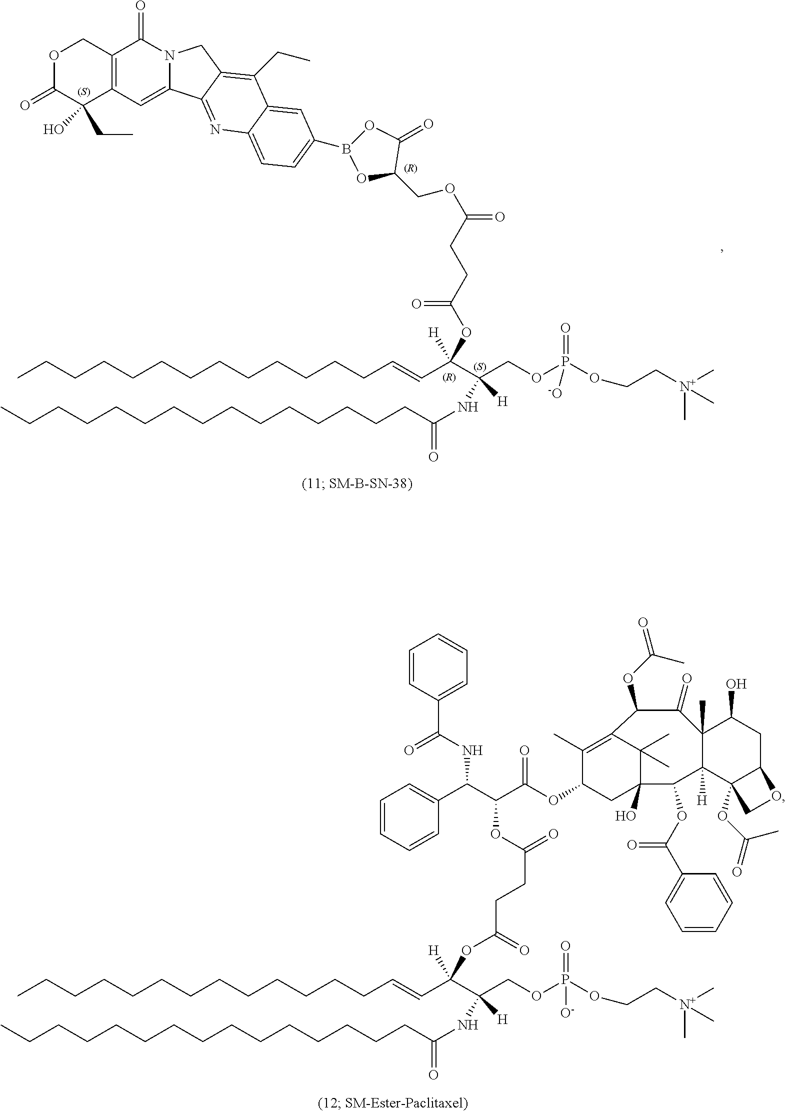

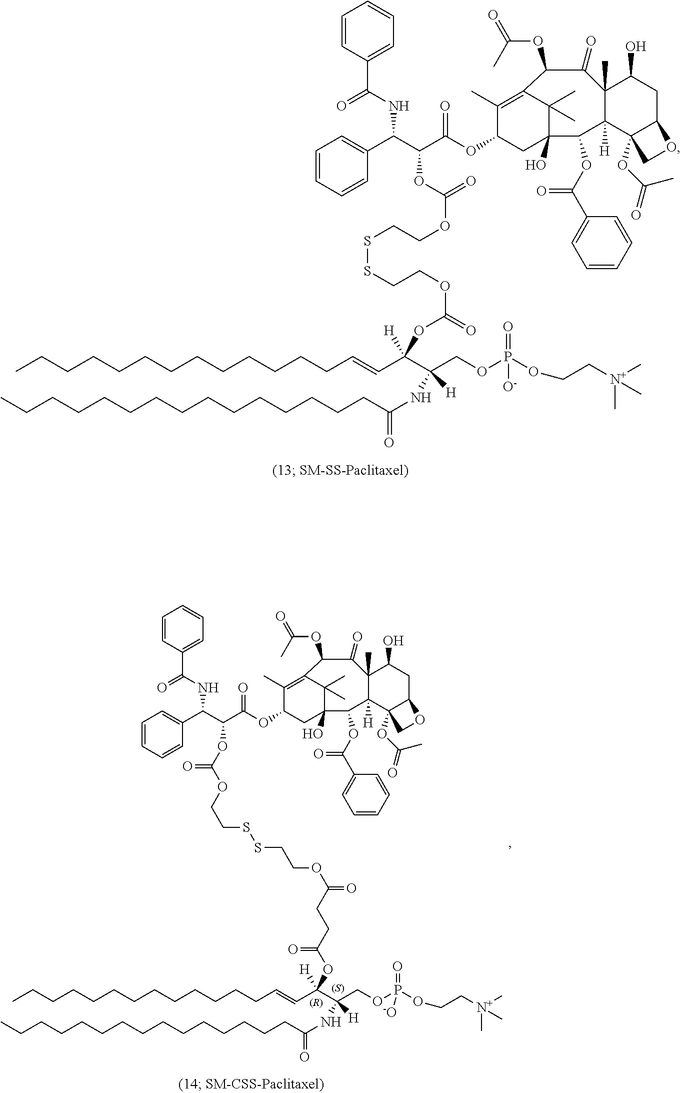

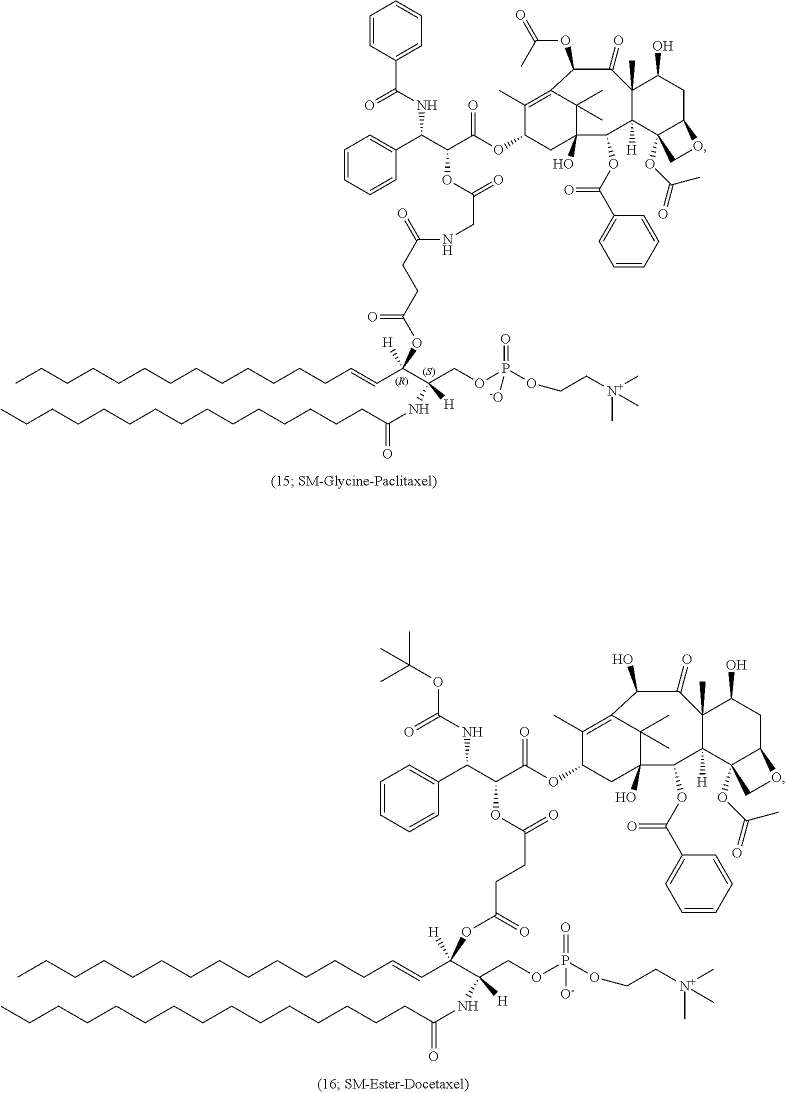

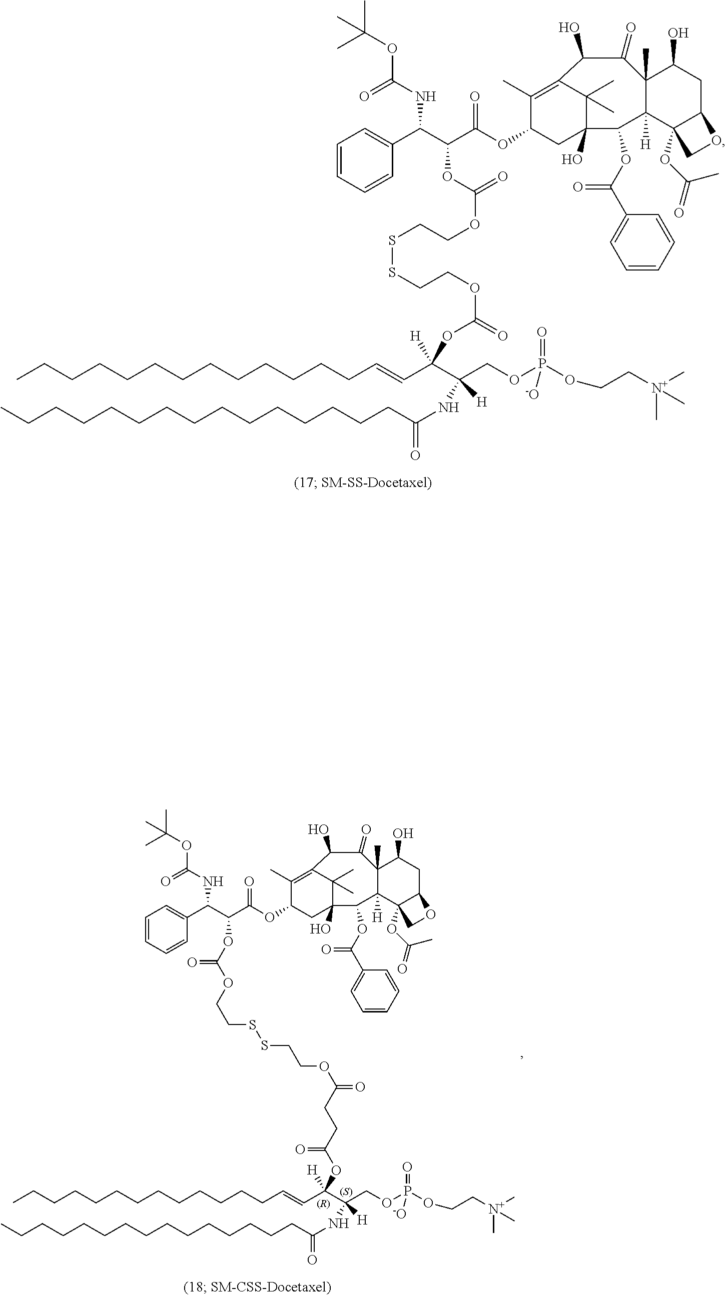

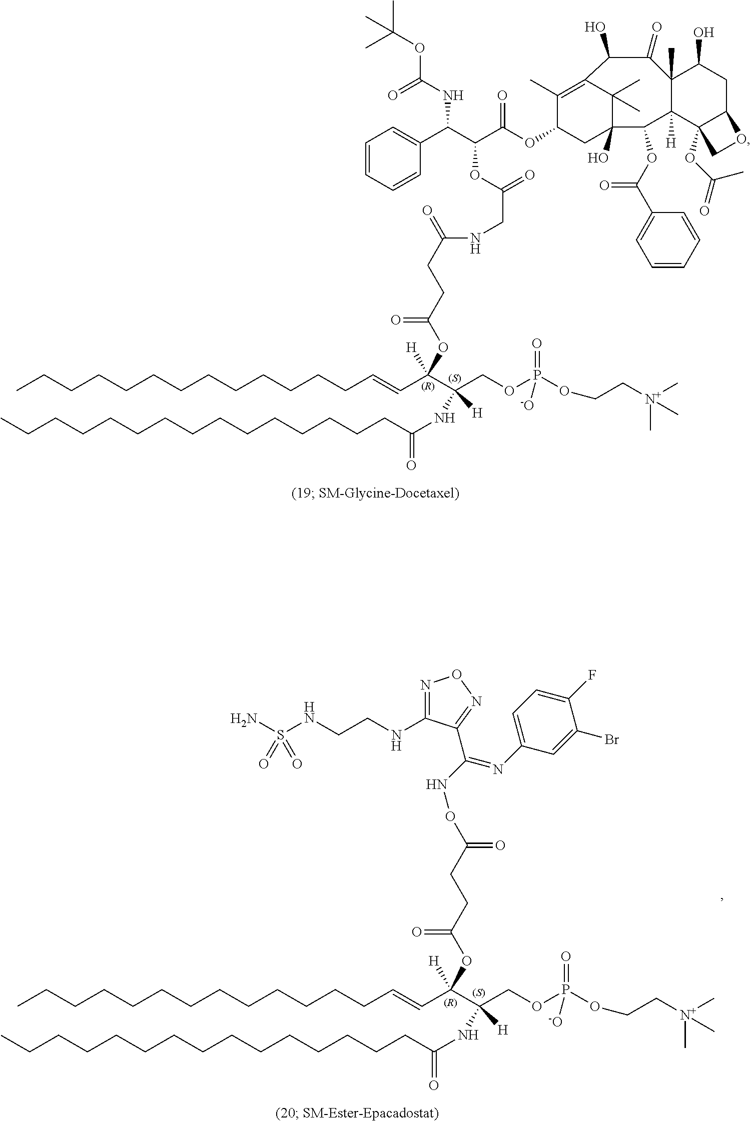

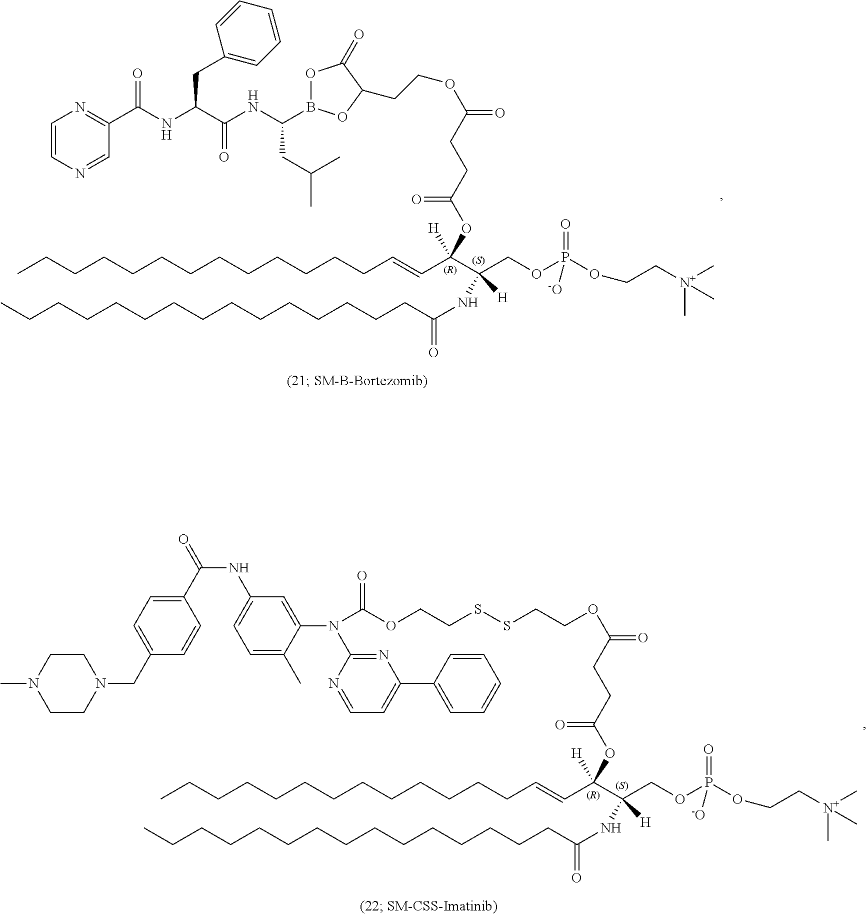

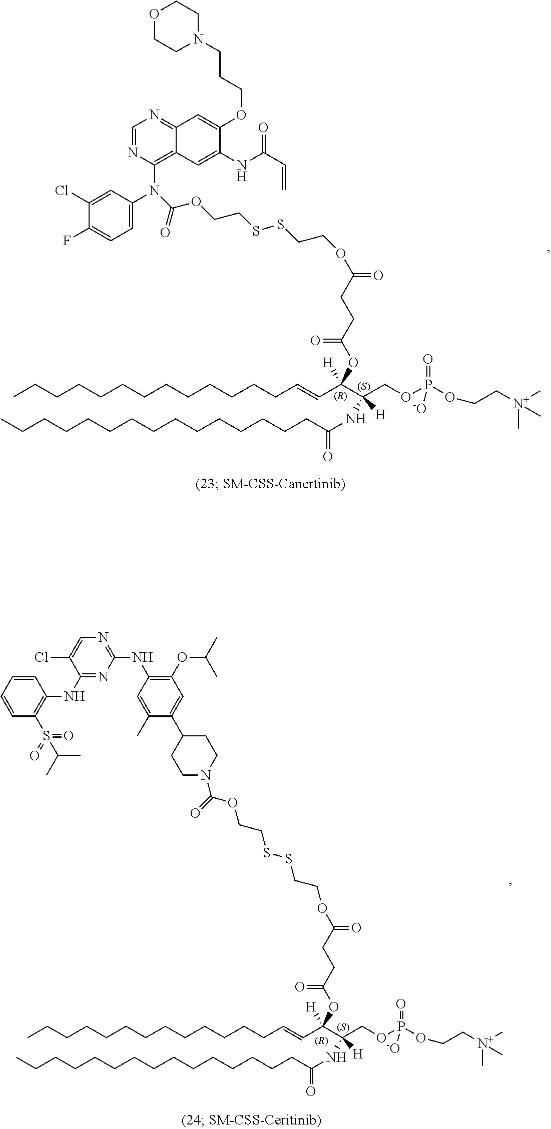

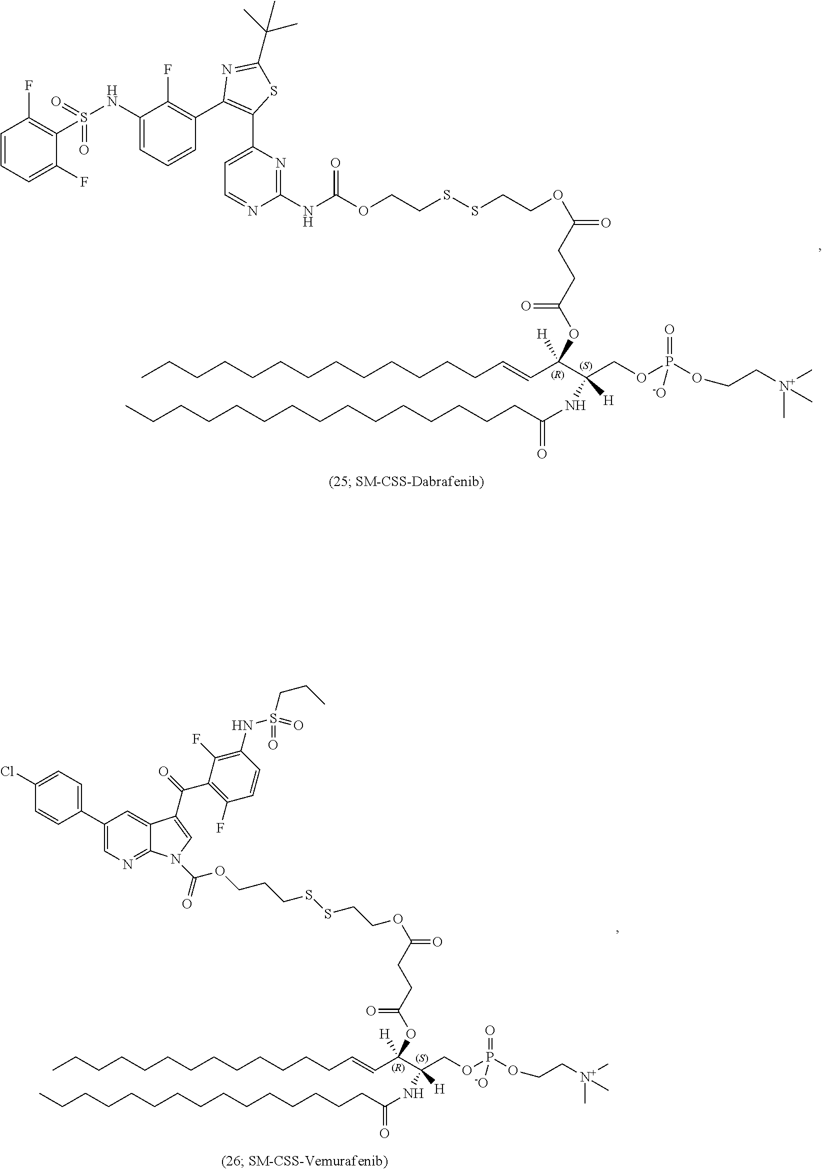

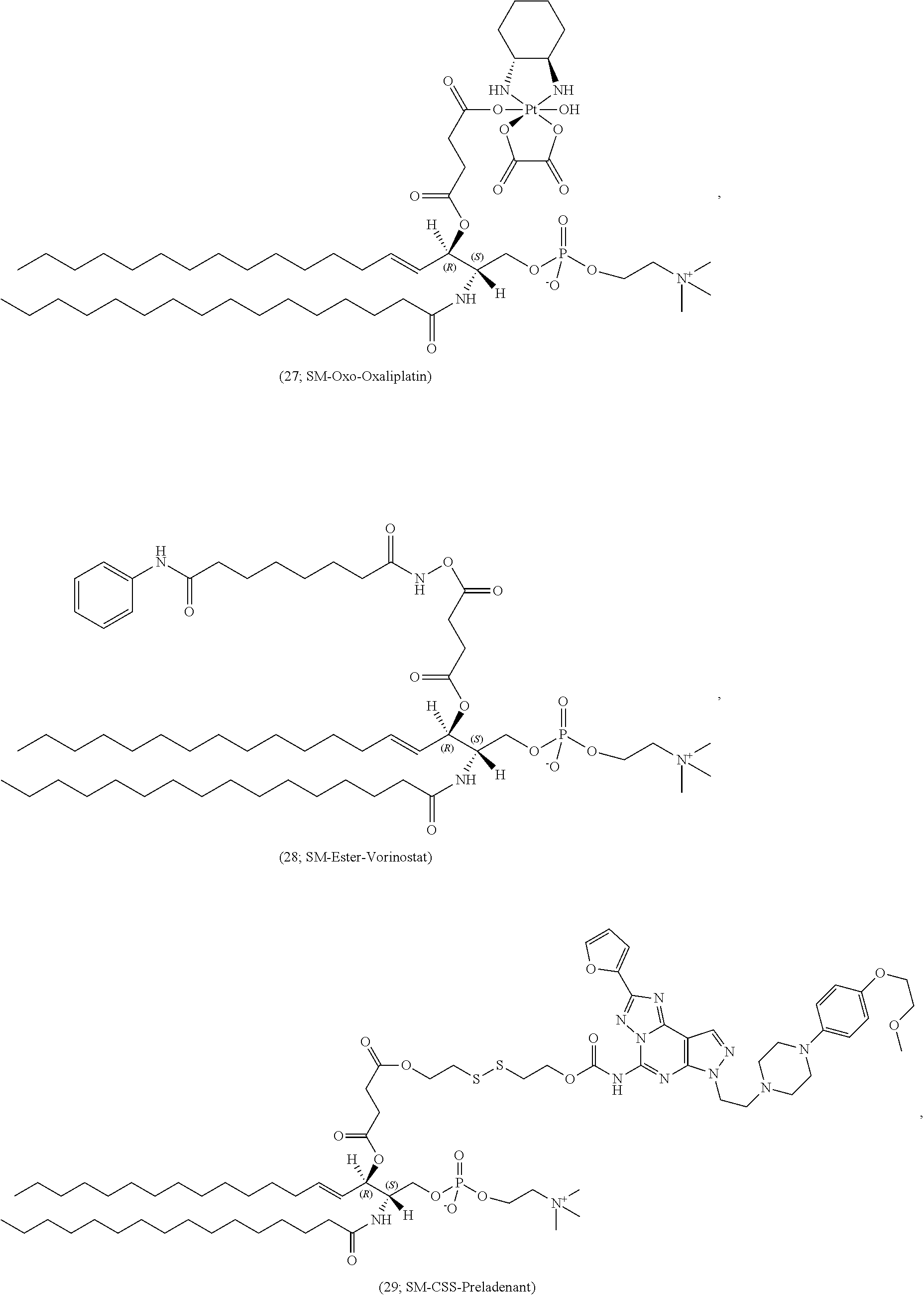

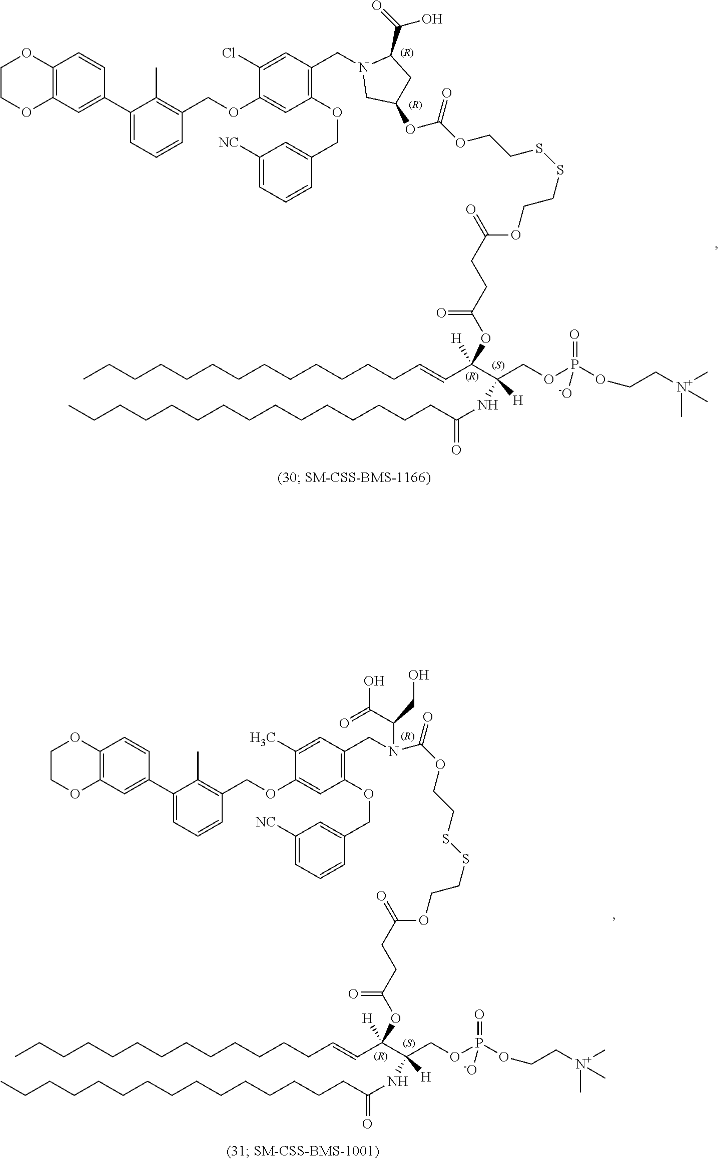

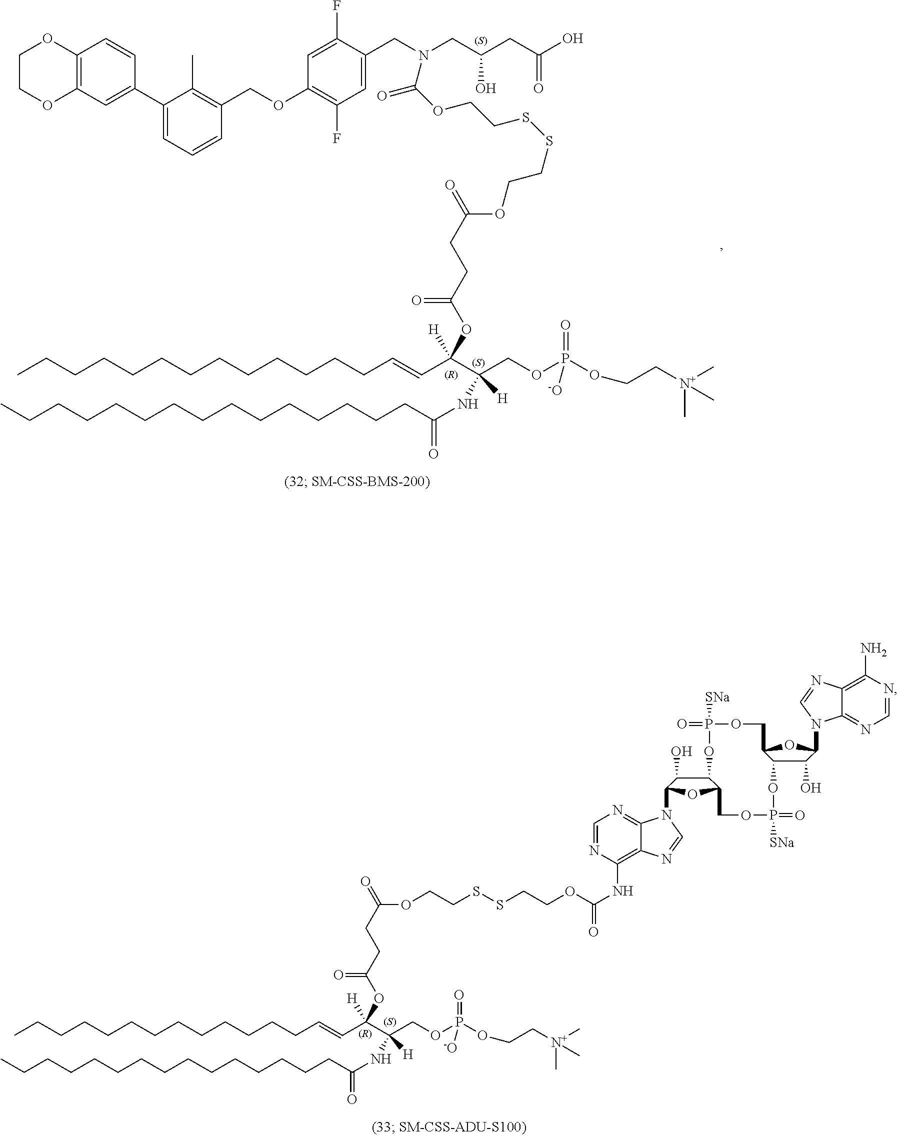

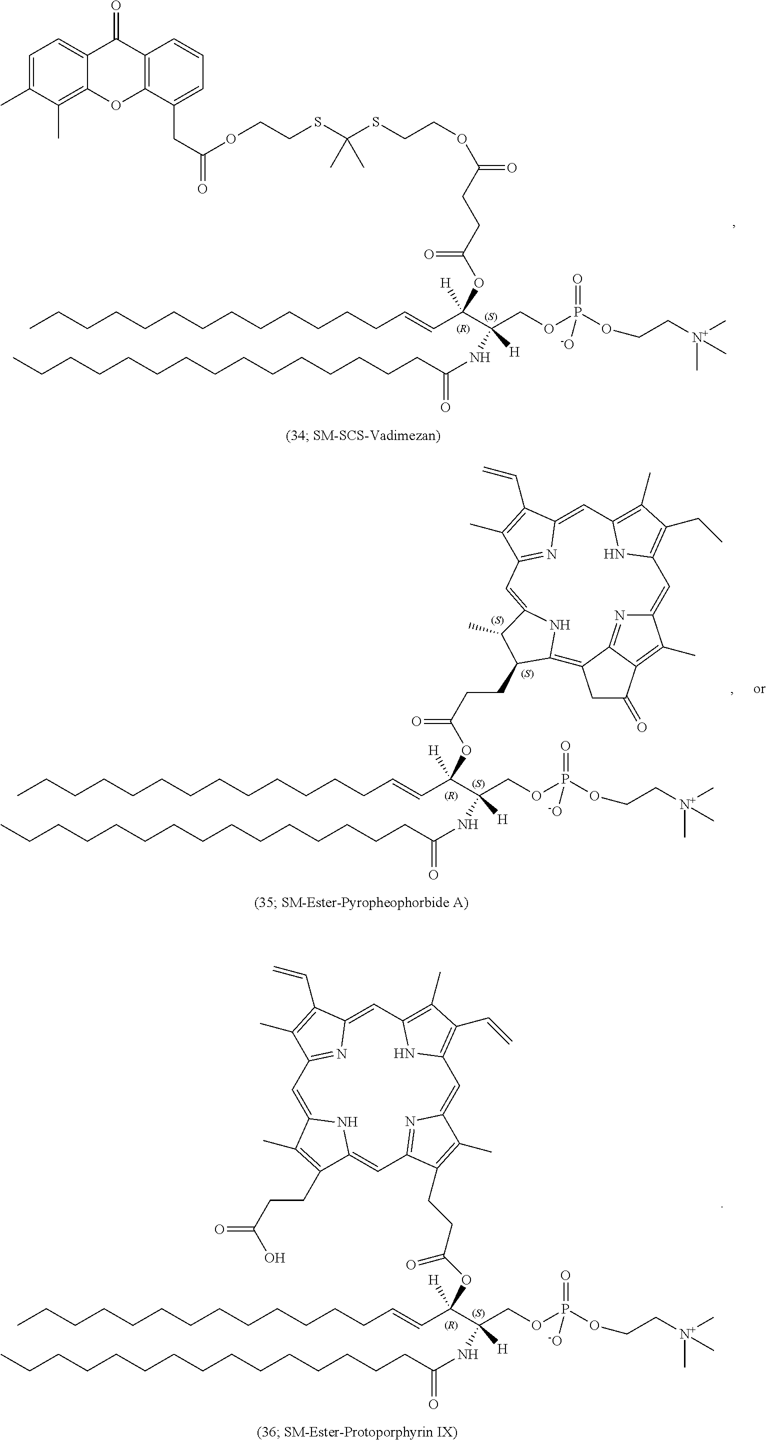

8. A doxorubicin (DOX)-drug conjugate comprising Formula (VII)-(VII): 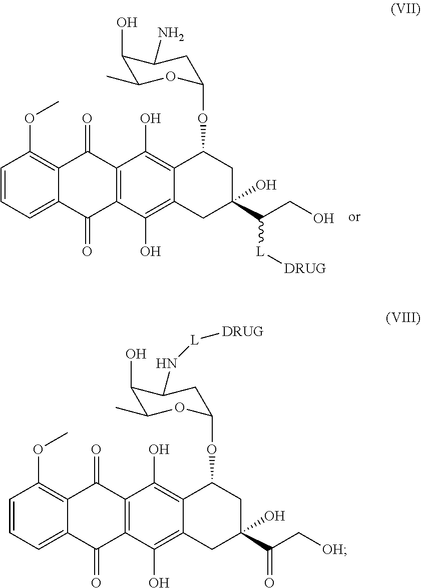
wherein:
L is a linker moiety; and
Drug is an anti-cancer drug.

9. The DOX-drug conjugate of claim 8, wherein the anti-cancer drug is hydrophobic or hydrophilic.

10. The DOX-drug conjugate of claim 8, wherein anti-cancer drug is selected from: indoximod, bortezomib, epacadostat, imiquimod, imatinib, canertinib, ceritinib, dabrafenib, vemurafenib, vorinostat, ADU-S100, amrubicin, AZD4635, BMS-1001, BMS-1166, BMS-200, BMS202, BMS-242, CA170, cabazitaxel, cabozantinib, camptothecin (CPT), capecitabine, carboplatin, cisplatin, dacarbazine, darolutamide, degarelix, digitoxin, digoxin, docetaxel, eribulin, etoposide, 5-fluorouracil, gemcitabine, irinotecan, ixabepilone, lenvatinib, methotrexate, mitoxantrone, NIR178, NLG919, oxaliplatin, paclitaxel, pazopanib, pemetrexed, preladenant, septacidin, SN-38, sorafenib, streptozocin, sunitinib, temozolomide, tipiracil, trifluridine, vadimezan, vinblastine, vincristine, vinorelbine, vipadenant, or combinations thereof.

11. The DOX-drug conjugate of claim 8, wherein the DOX-drug conjugate comprises Formula (IX)-(XVIII): 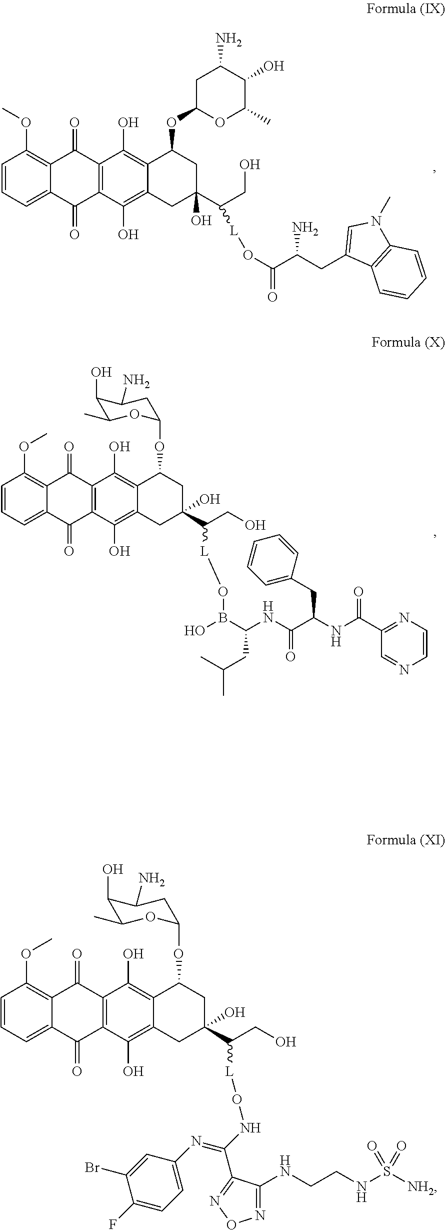 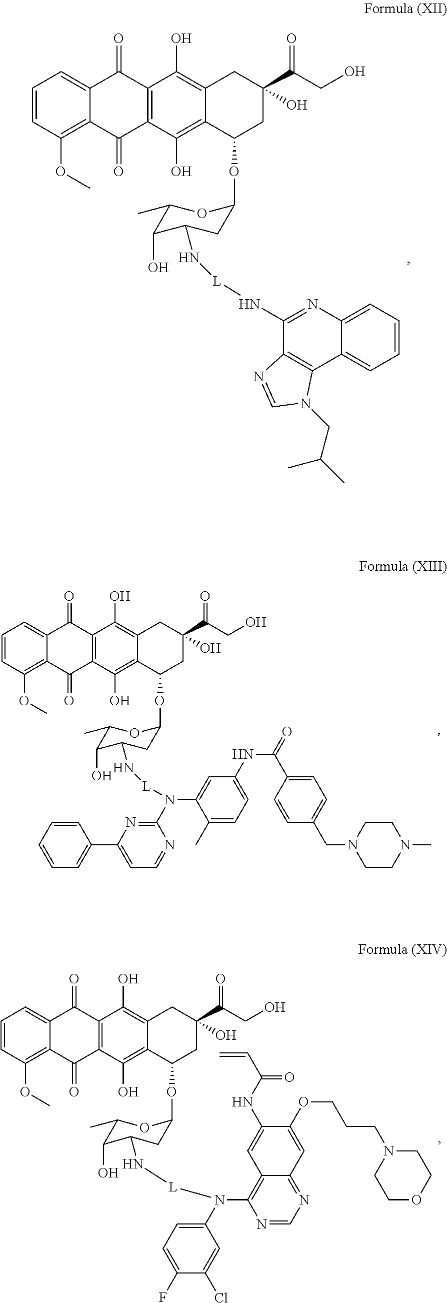 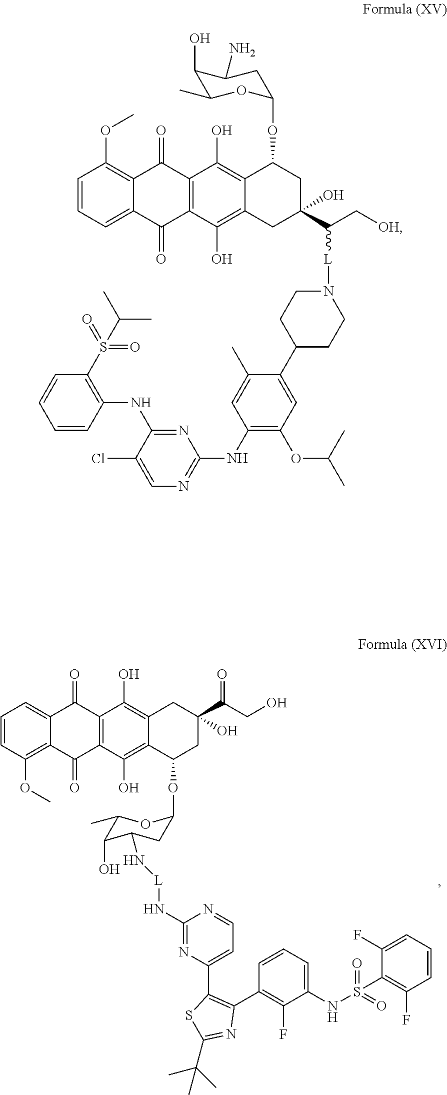 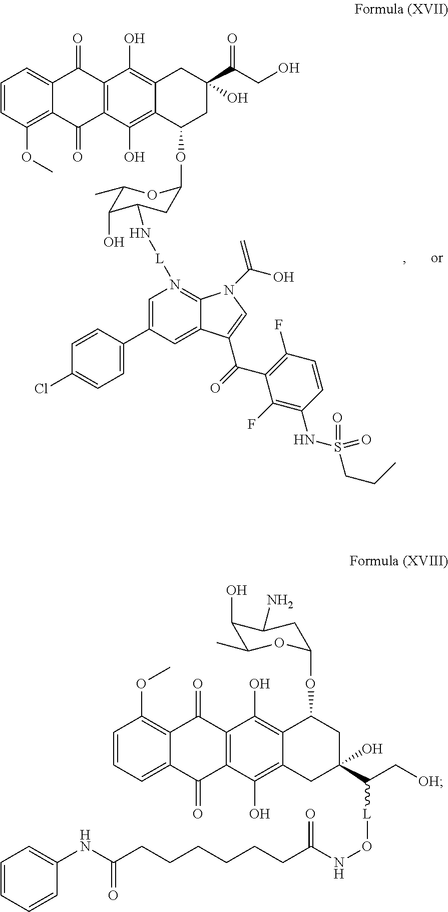
wherein L is a linker moiety.

12. The DOX-drug conjugate of claim 8, wherein L is selected from: 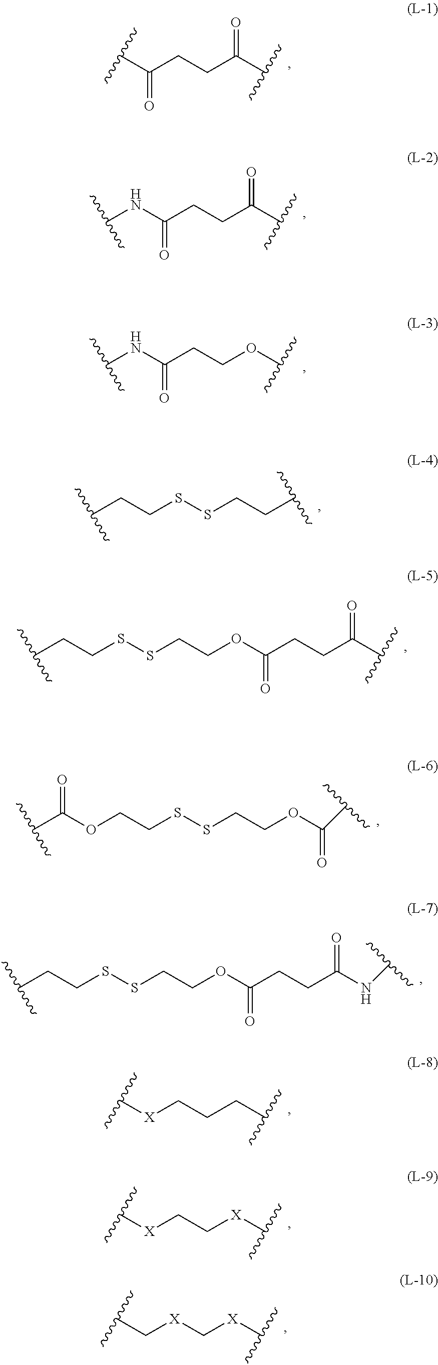 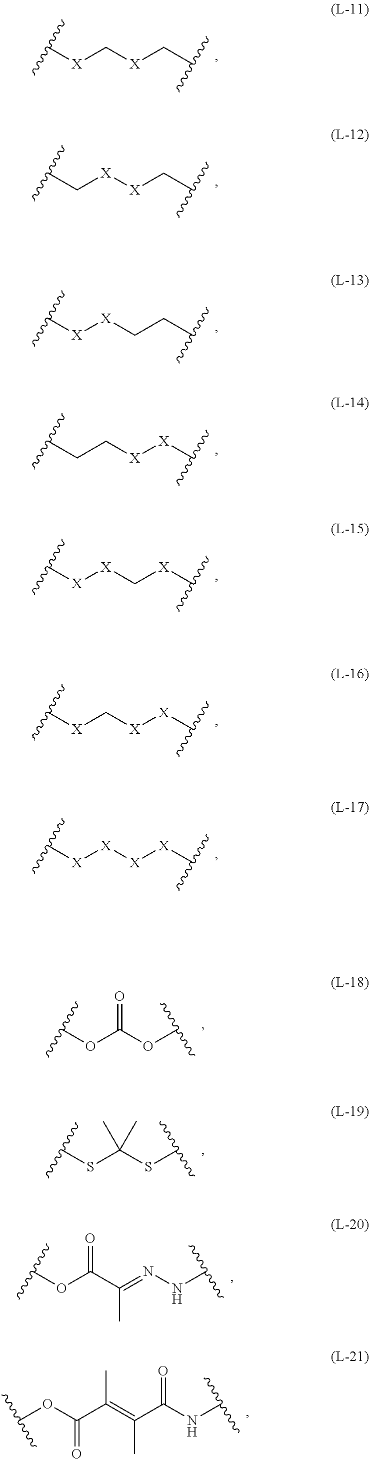 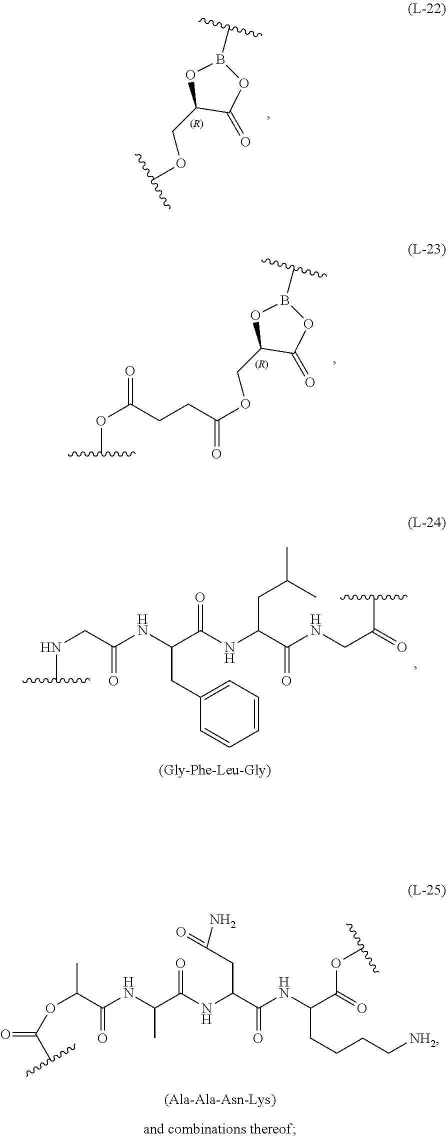
wherein X is independently, O, S, —NH, or —CO.

13. The DOX-drug conjugate of claim 8, wherein the DOX-drug conjugate is:

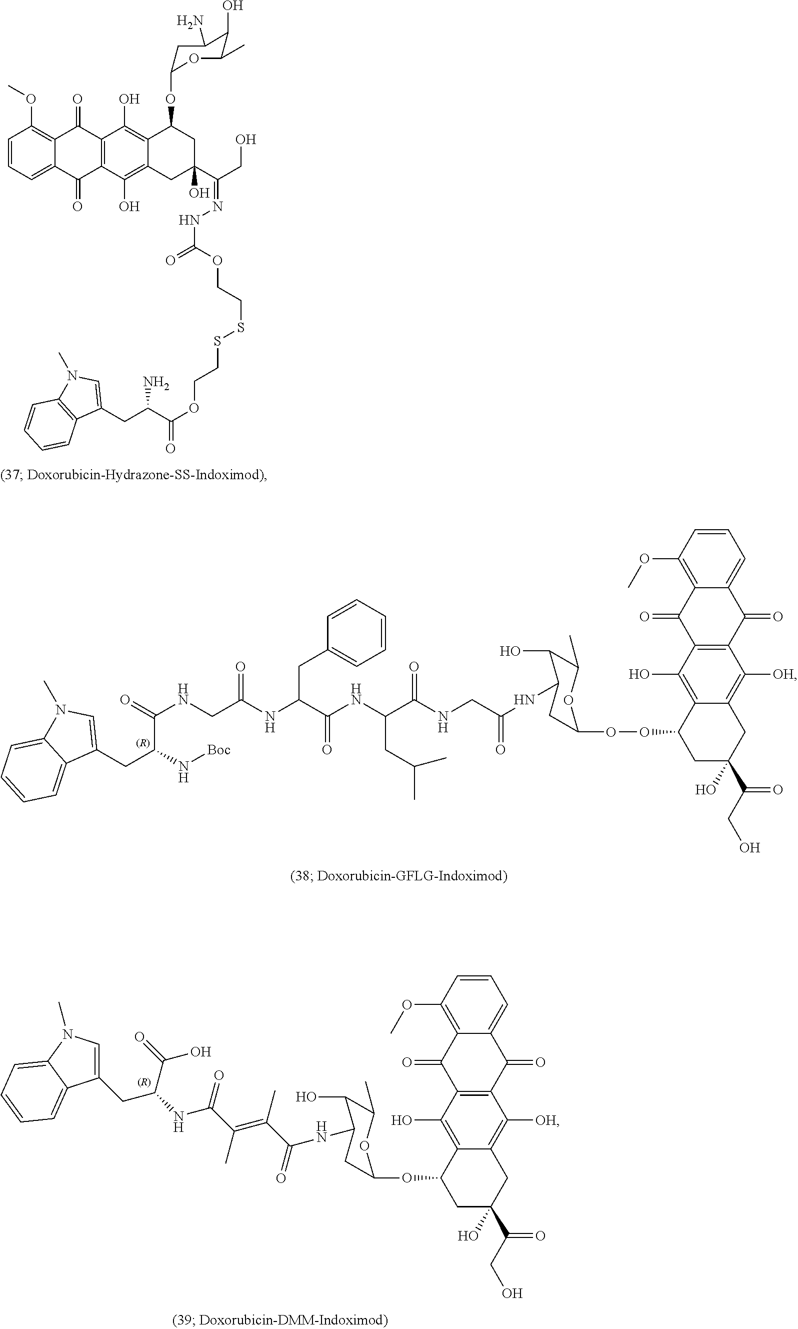

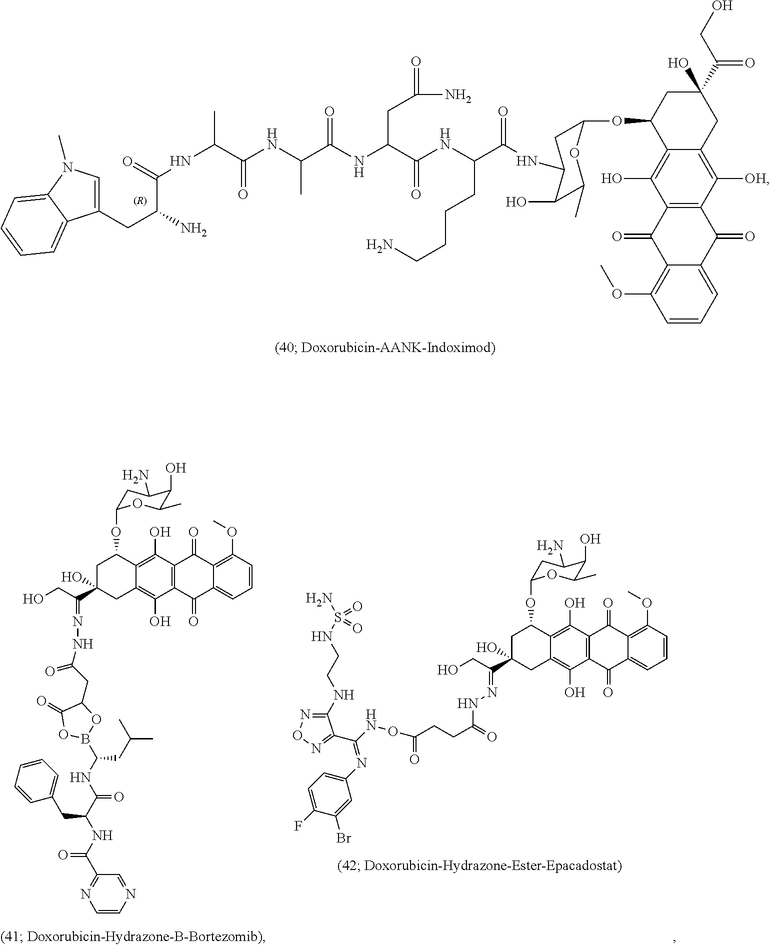

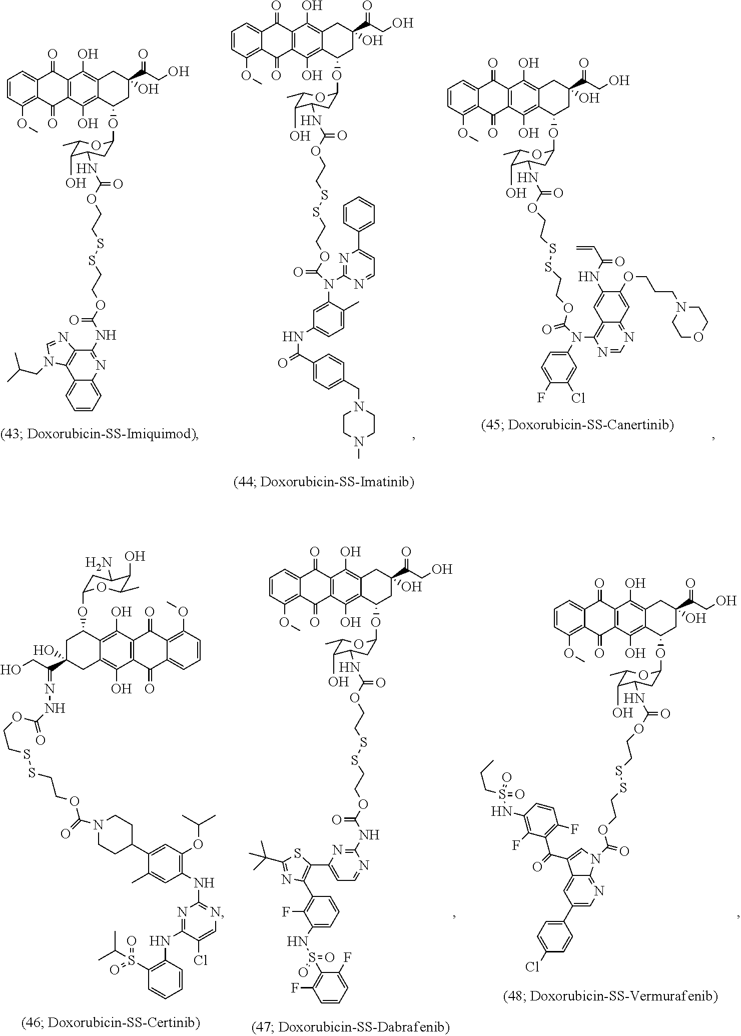

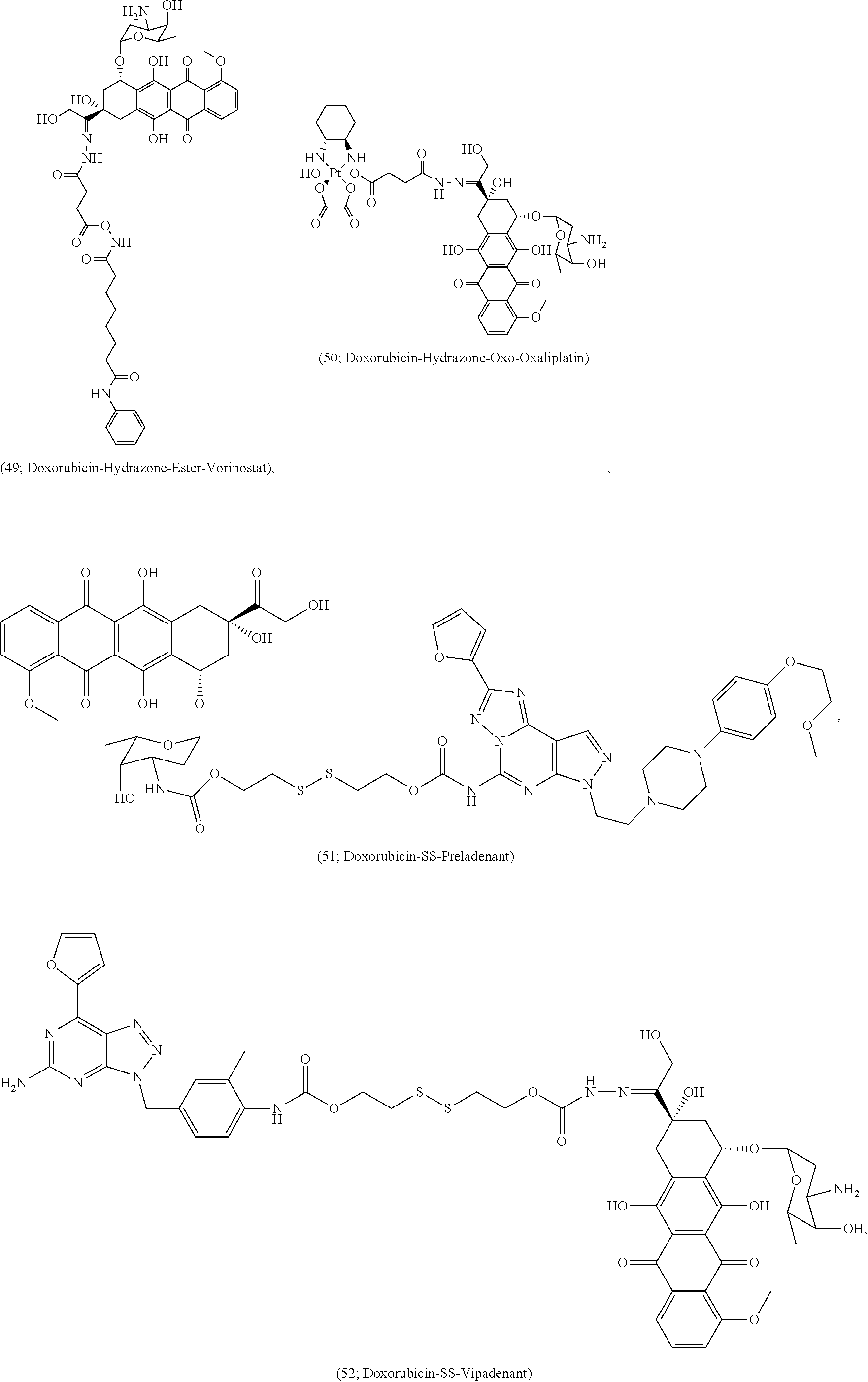

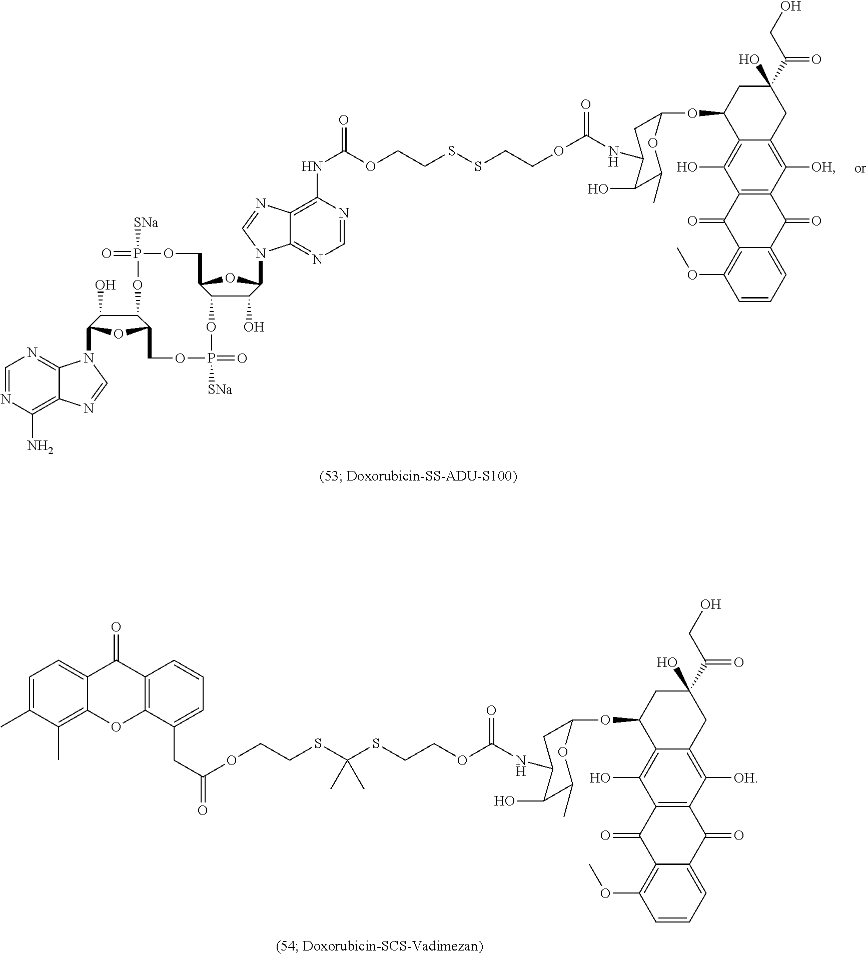

14. A nanovesicle comprising a lipid bilayer including a sphingomyelin-drug conjugate comprising Formula (I): 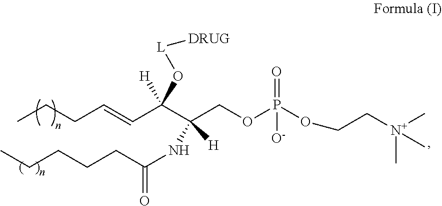
wherein each n is independently 5 to 20;
L is a linker moiety; and
Drug is an anti-cancer drug.

15. The nanovesicle of claim 14, wherein the sphingomyelin-drug conjugate comprises Formula (II): 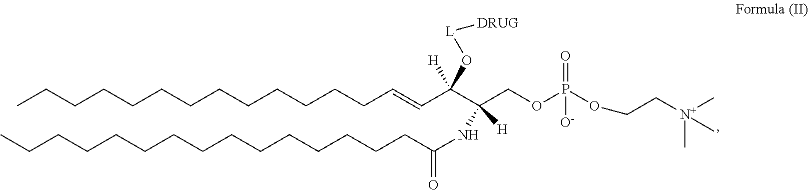
wherein L is a linker moiety; and
Drug is an anti-cancer drug.

16. The nanovesicle of claim 14, wherein the anti cancer drug is hydrophilic or hydrophobic.

17. The nanovesicle of claim 14, wherein the anti-cancer drug is selected from the group consisting of: camptothecin (CPT), paclitaxel, docetaxel, ADU-S100, amrubicin, 5-aminolevulinic acid, AZD4635, BMS-1001, BMS-1166, BMS-200, BMS-202, BMS-242, BMS-242, bortezomib, CA170, cabazitaxel, cabozantinib, canertinib, capecitabine, carboplatin, ceritinib, chlorin e6, cisplatin, dabrafenib, dacarbazine, darolutamide, daunorubicin, degarelix, digoxin, doxorubicin, epacadostat, epirubicin, eribulin, esorubicin, etoposide, fingolimod, 5-fluorouracil, galanthamine, gemcitabine, idarubicin, imatinib, imiquimod, indoximod, irinotecan, ixabepilone, lenvatinib, memantine, methotrexate, mitoxantrone, NIR178, NLG919, oxaliplatin, pazopanib, pemetrexed, preladenant, protoporphyrin IX (PPIX), pyropheophorbide-A (PPA), septacidin, SN-38, sorafenib, streptozocin, sunitinib, temozolomide, tipiracil, TPI-287, trifluridine, vadimezan, vemurafenib, vinblastine, vincristine, vinorelbine, vipadenant, vorinostat, and combinations thereof.

18. The nanovesicle of claim 14, wherein the sphingomyelin-drug conjugate comprises Formula (III)-(VI): 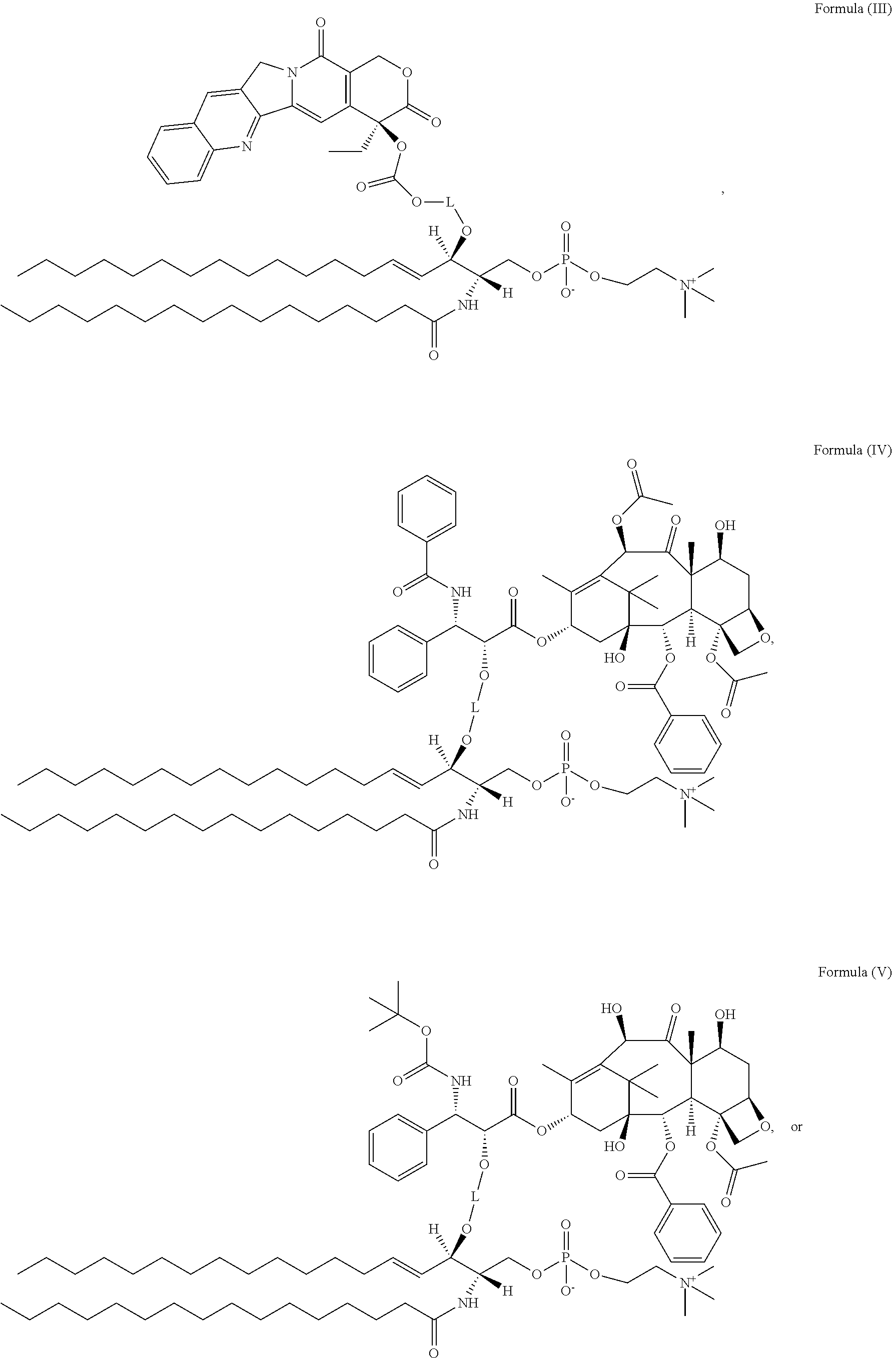 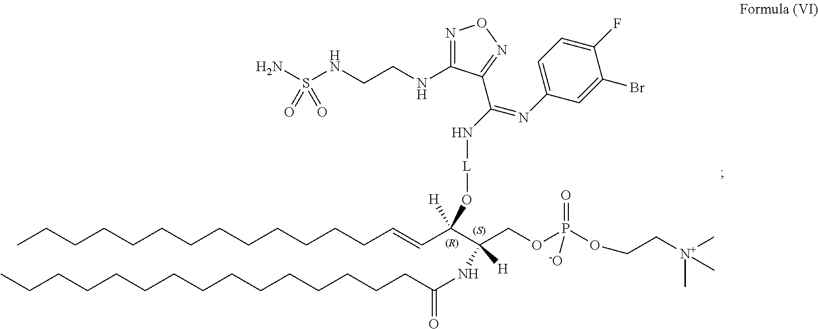
wherein L is a linker moiety.

19. The nanovesicle of claim 14, wherein L is selected from: 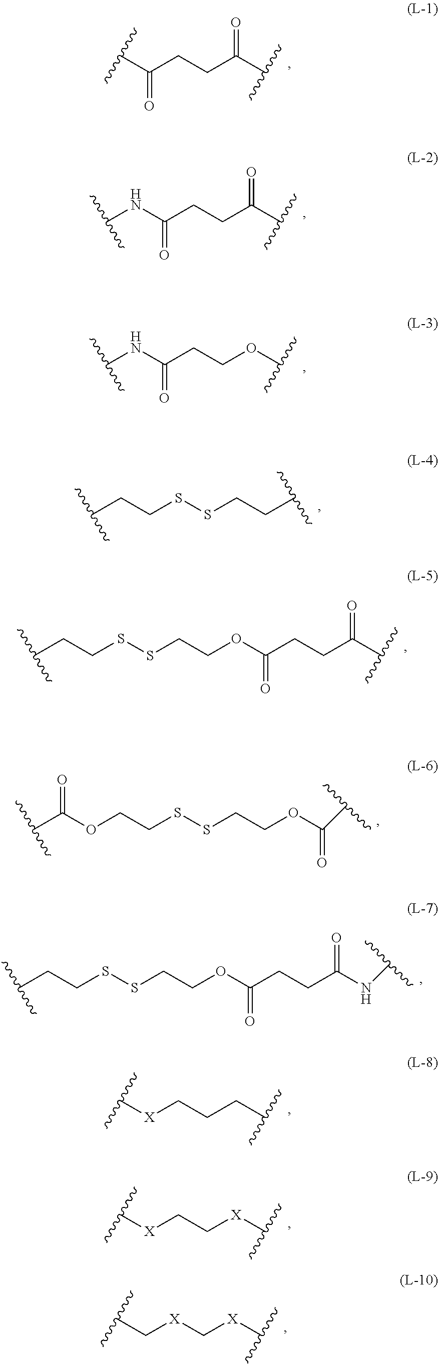 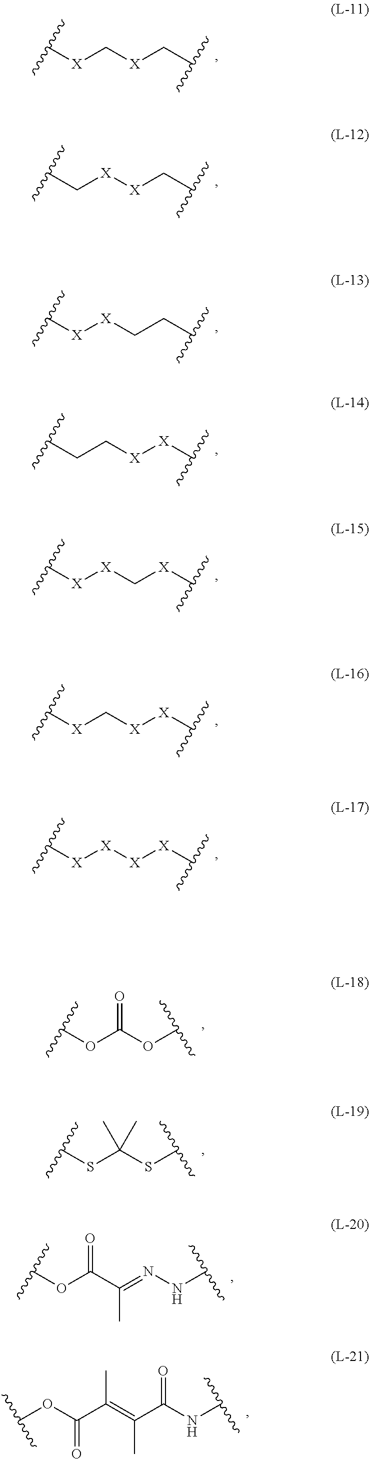 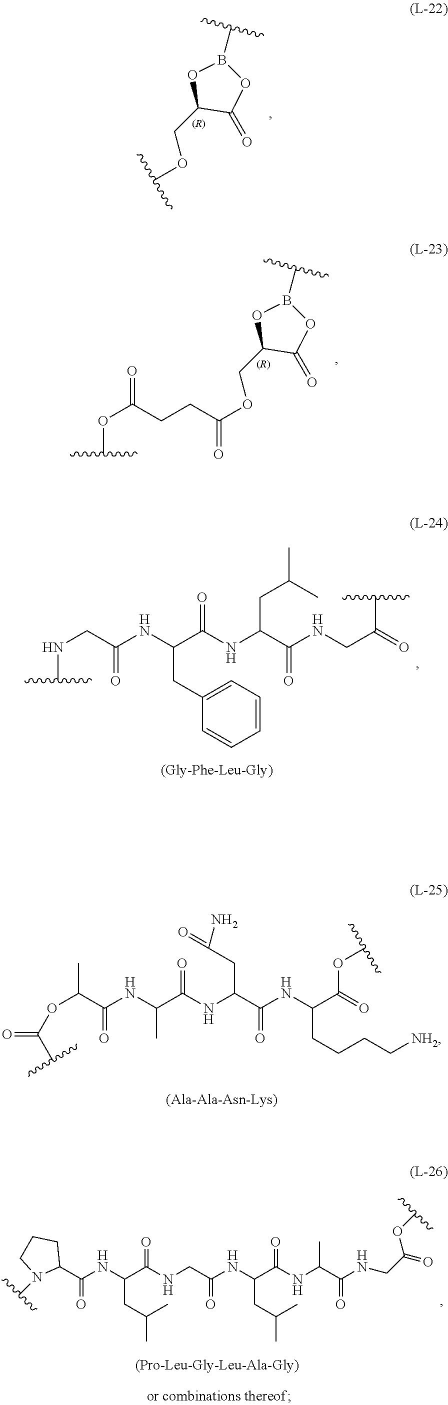
wherein X is independently, O, S, —NH, or —CO.

20. The nanovesicle of claim 14, wherein the sphingomyelin-drug conjugate is:

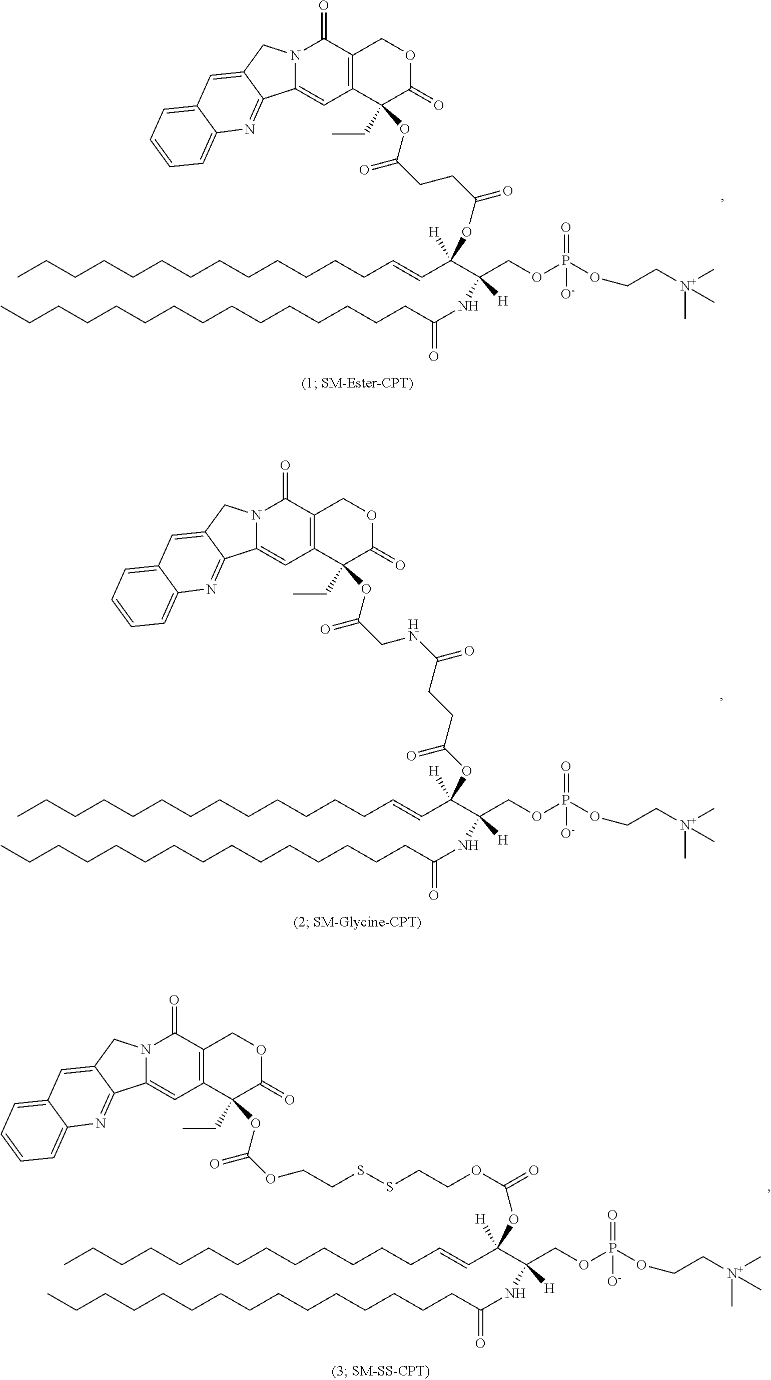

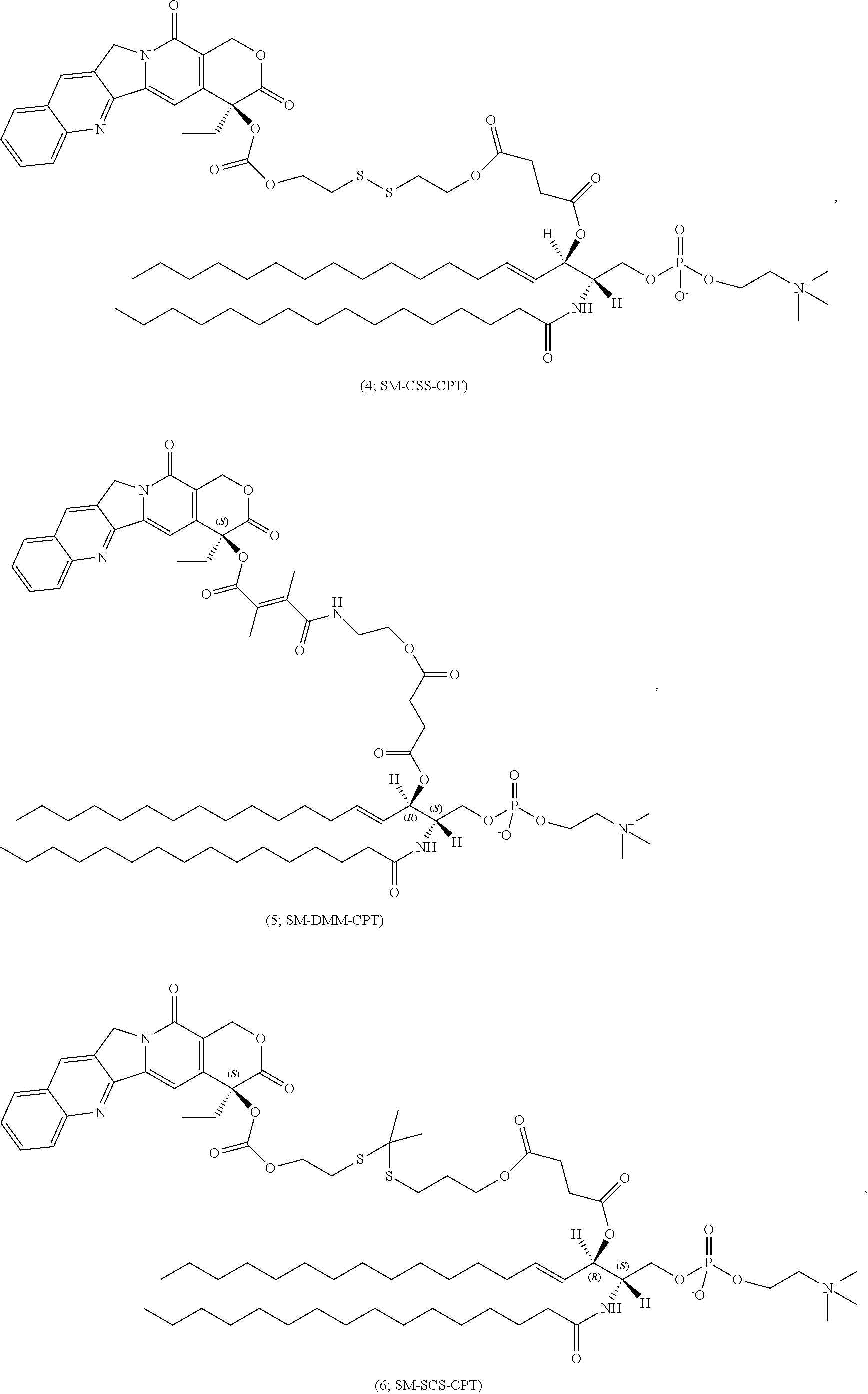

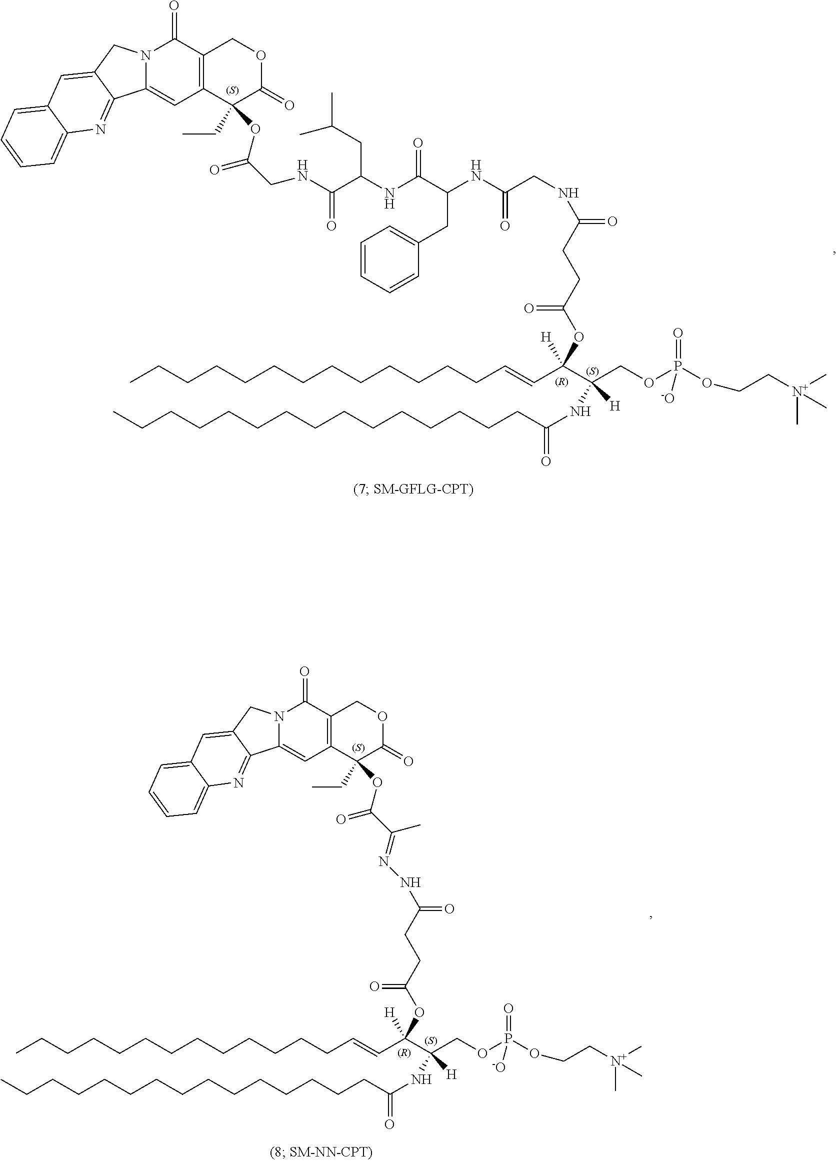

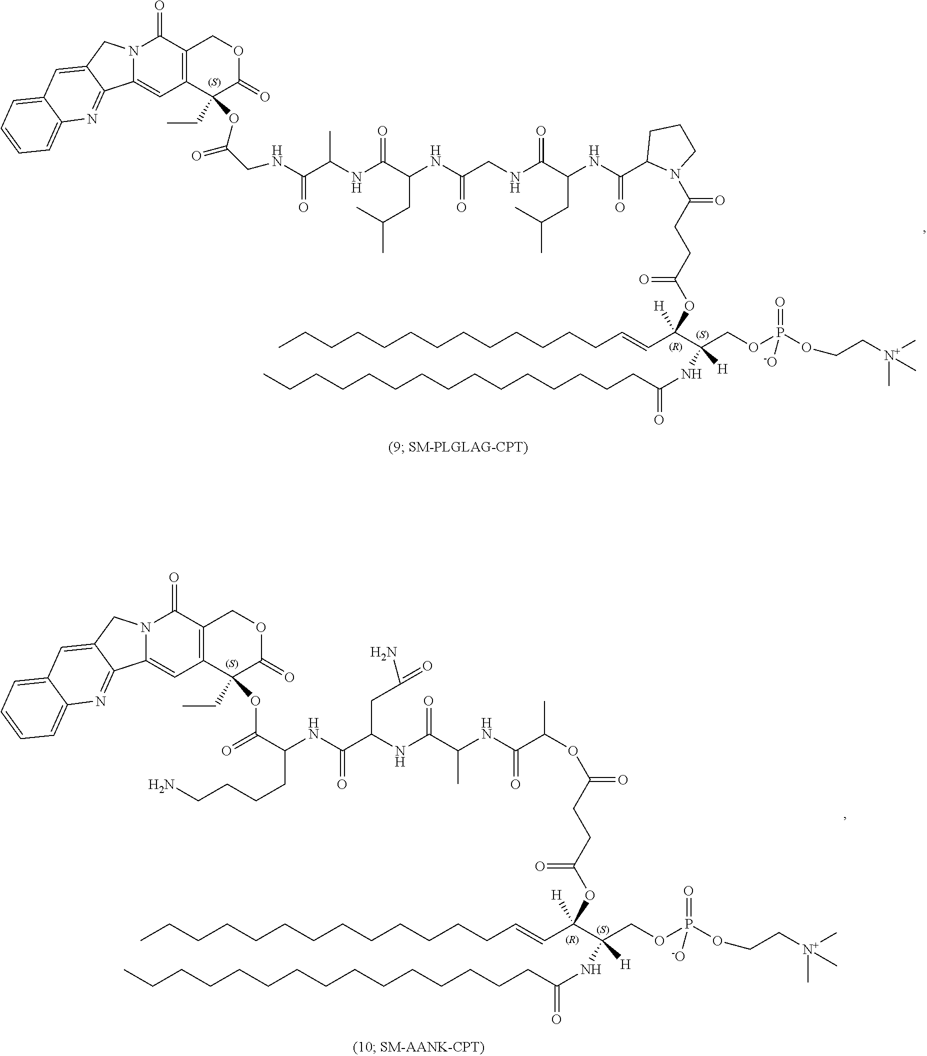

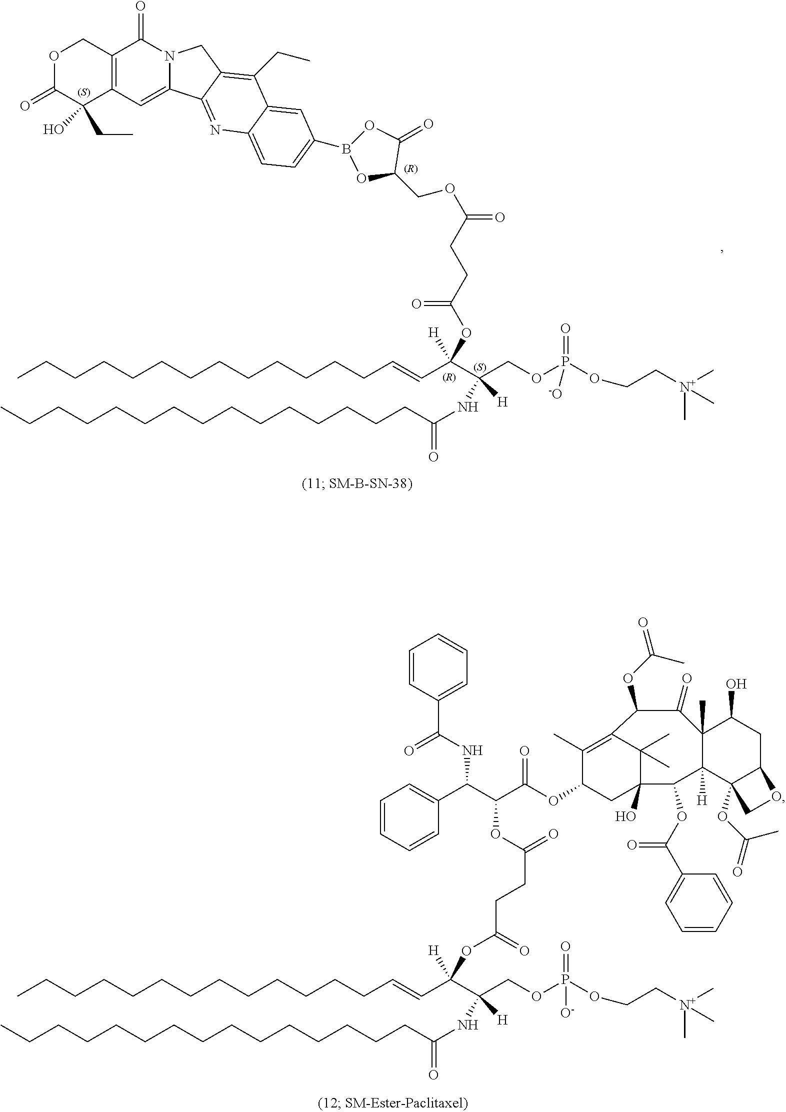

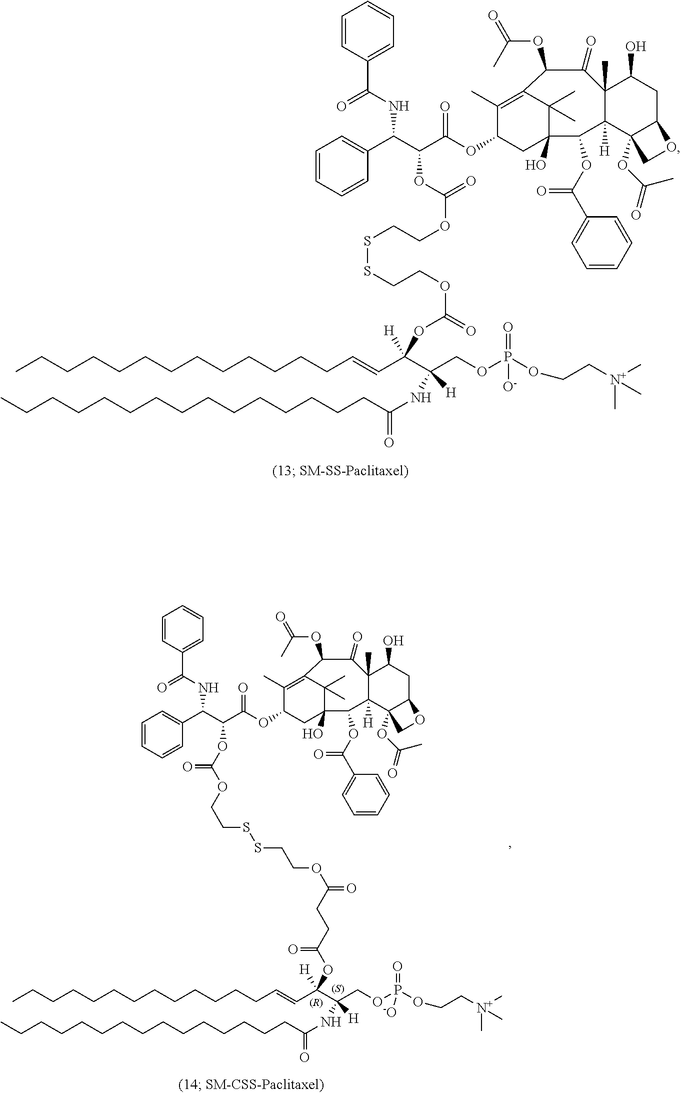

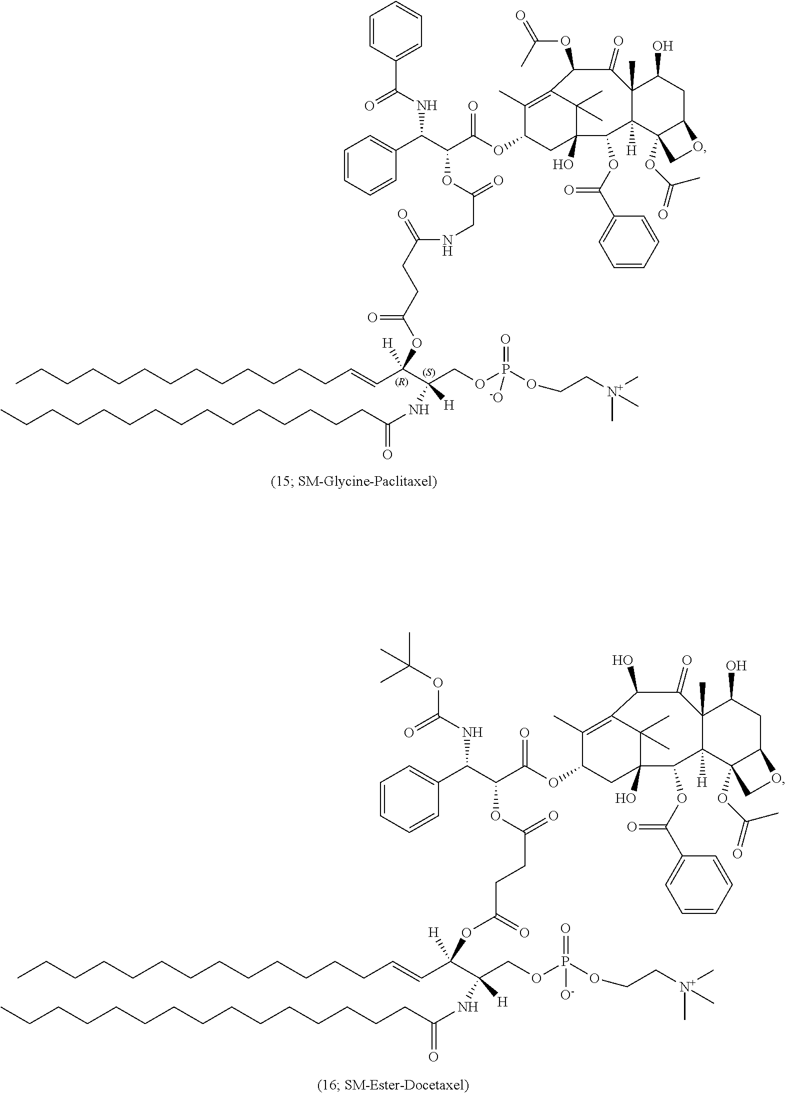

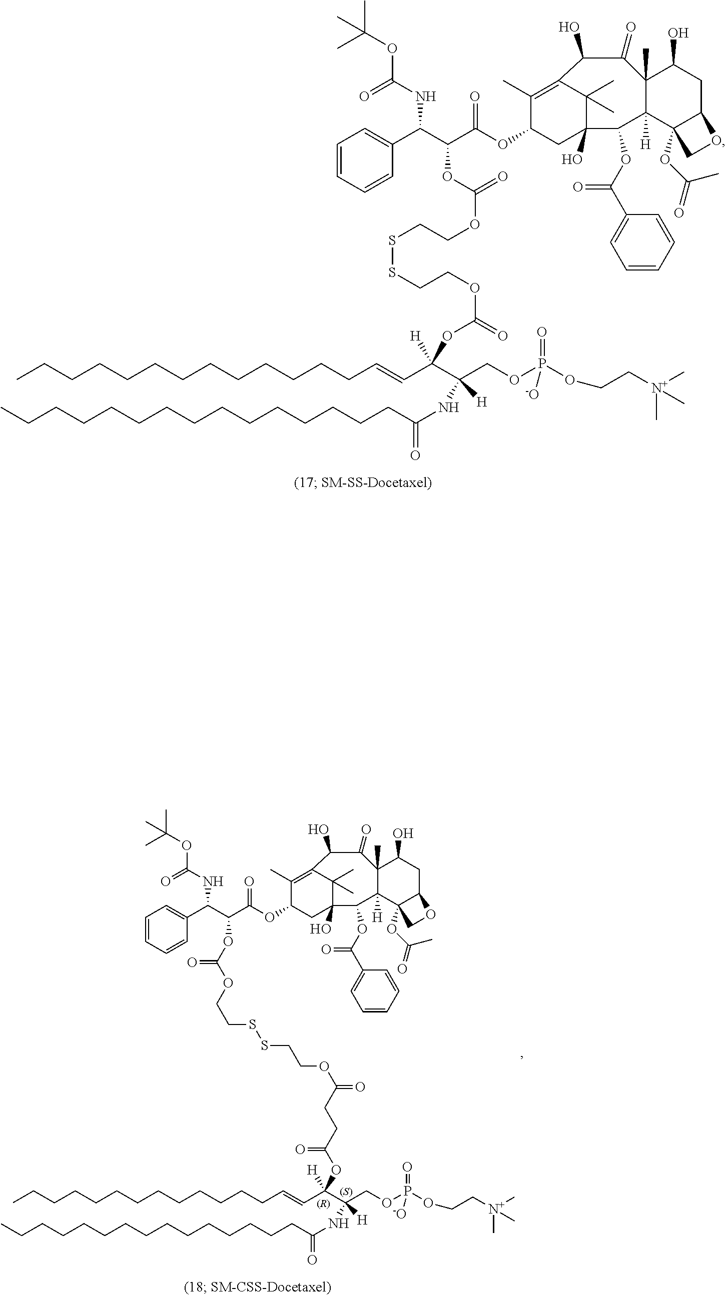

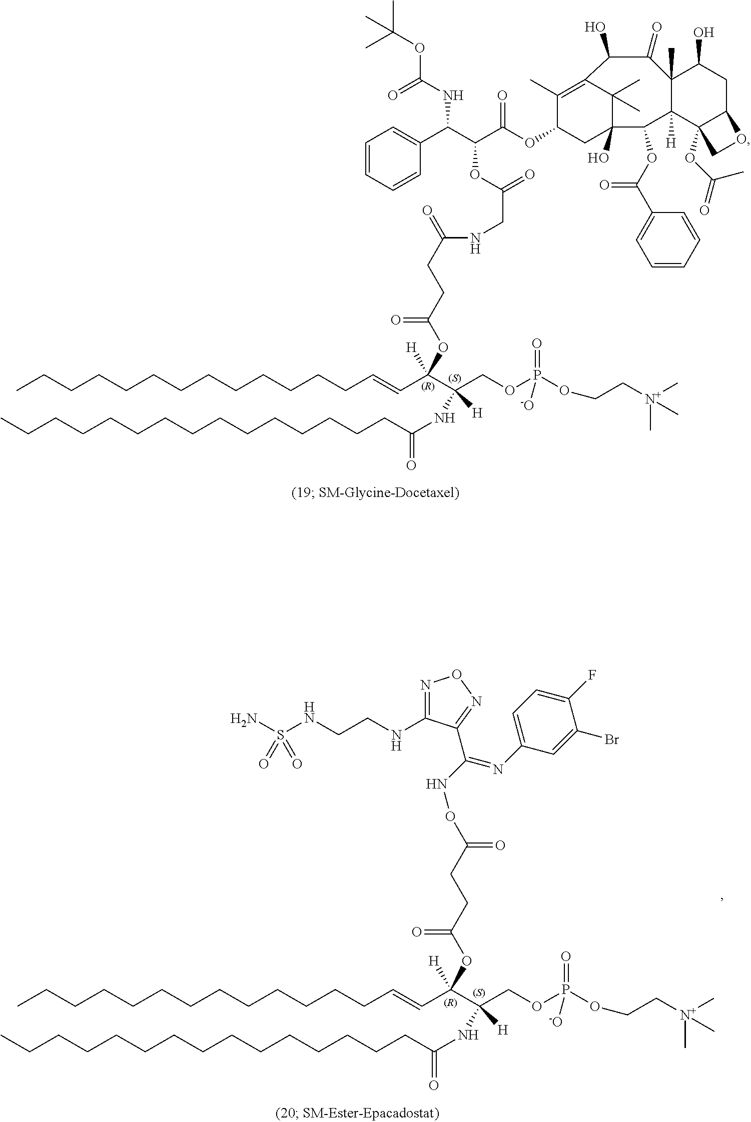

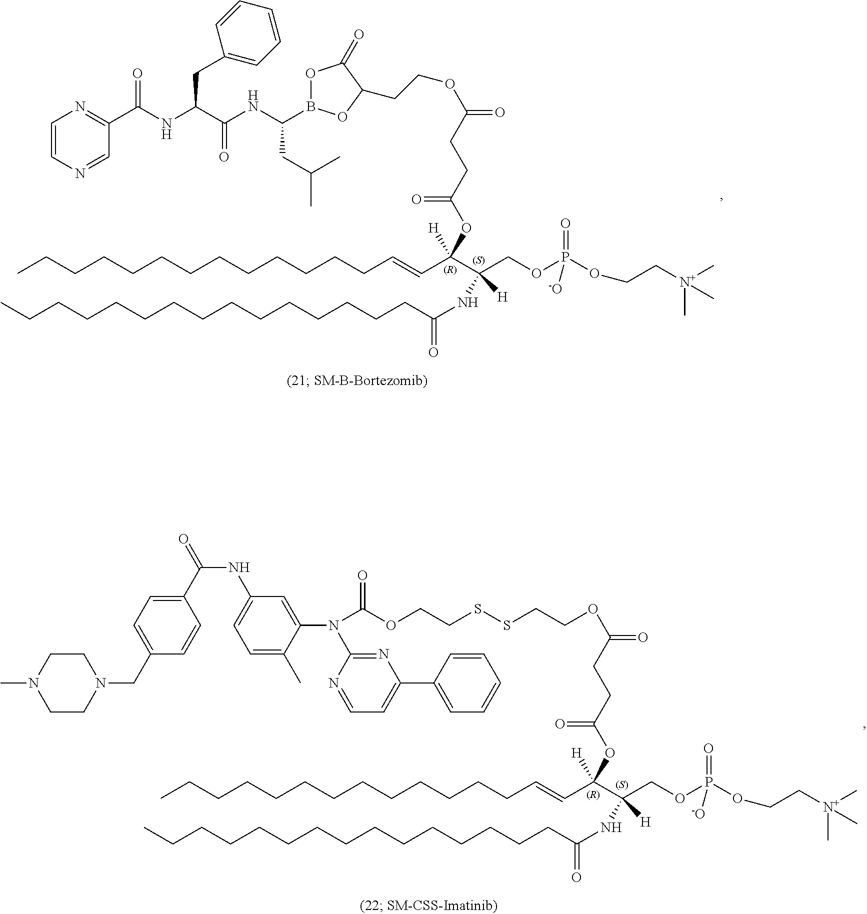

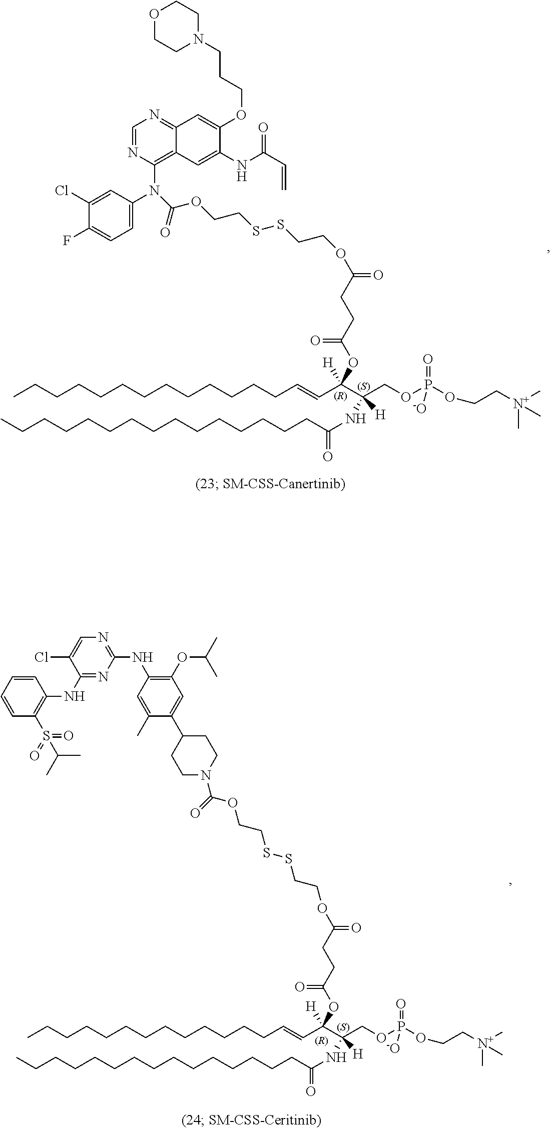

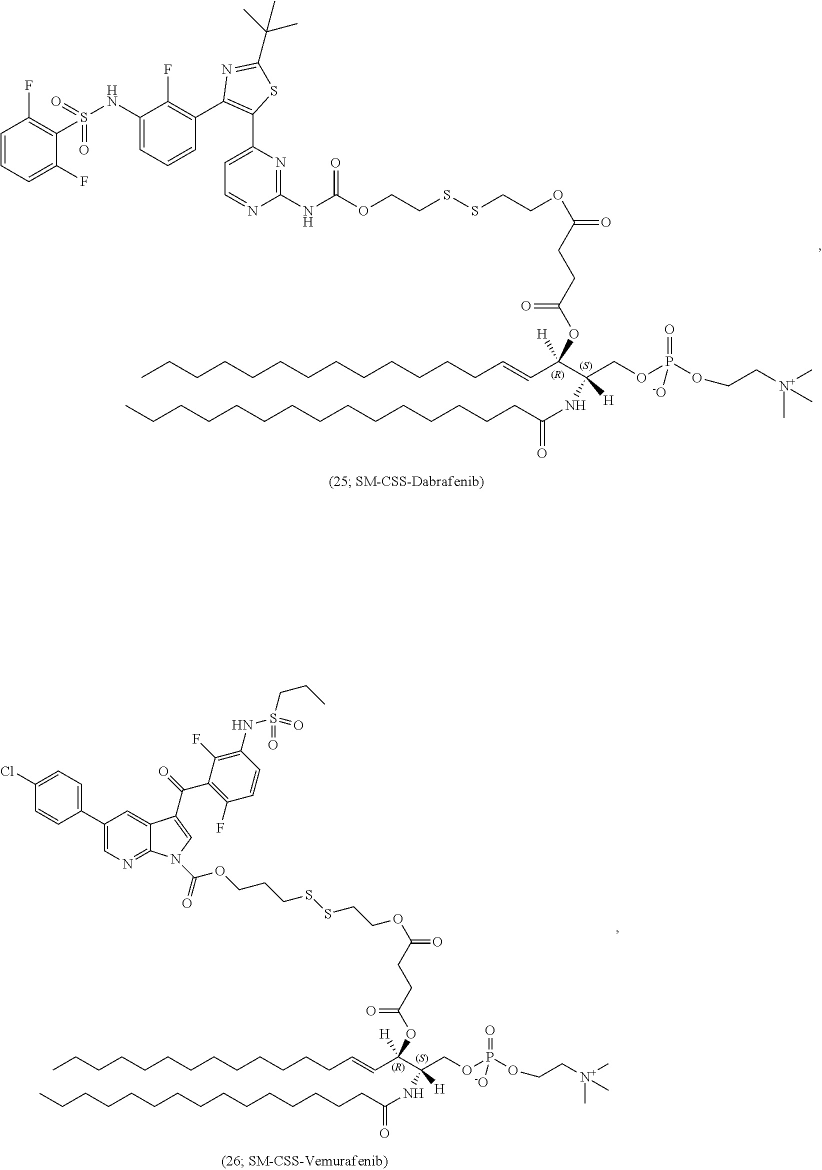

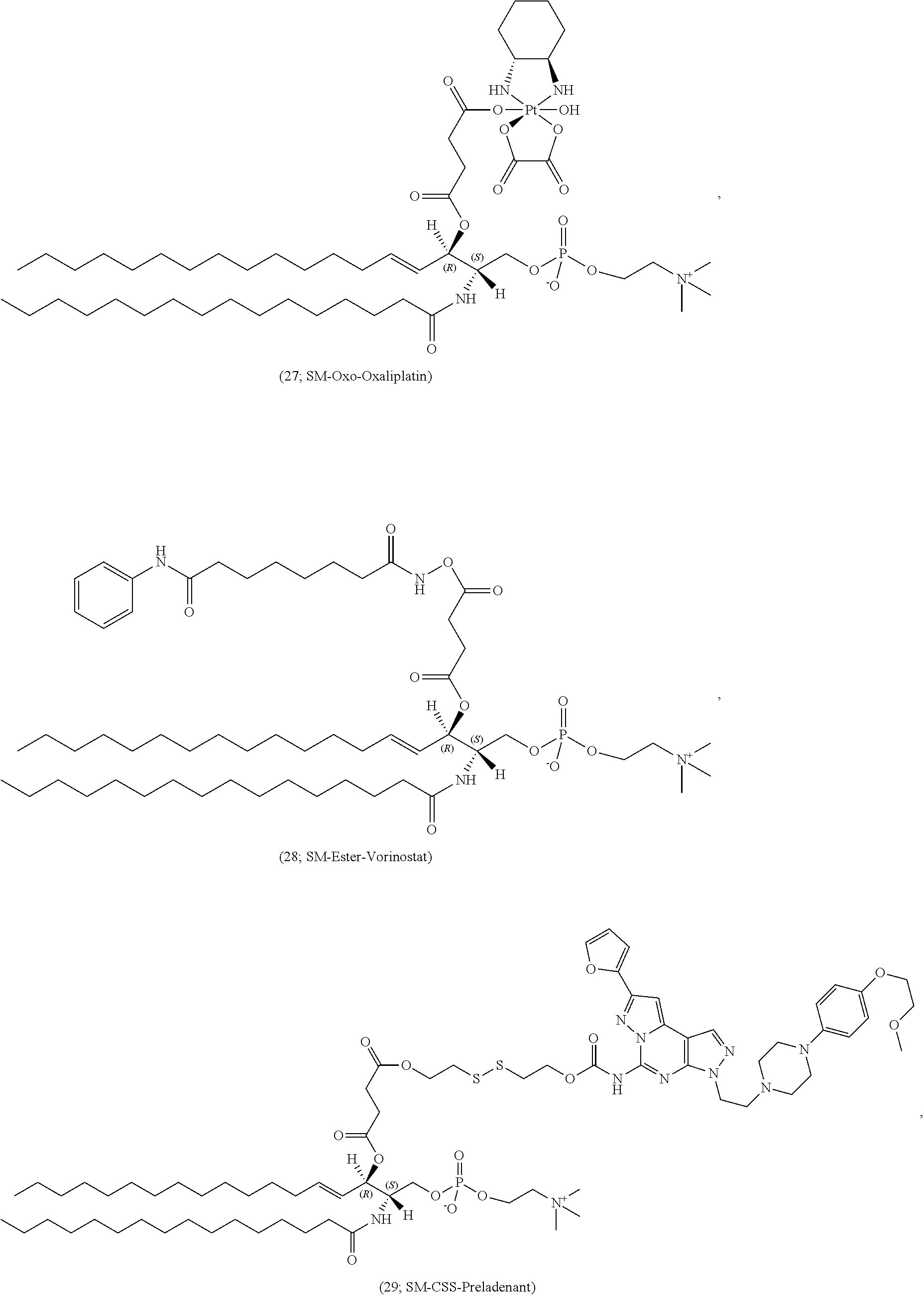

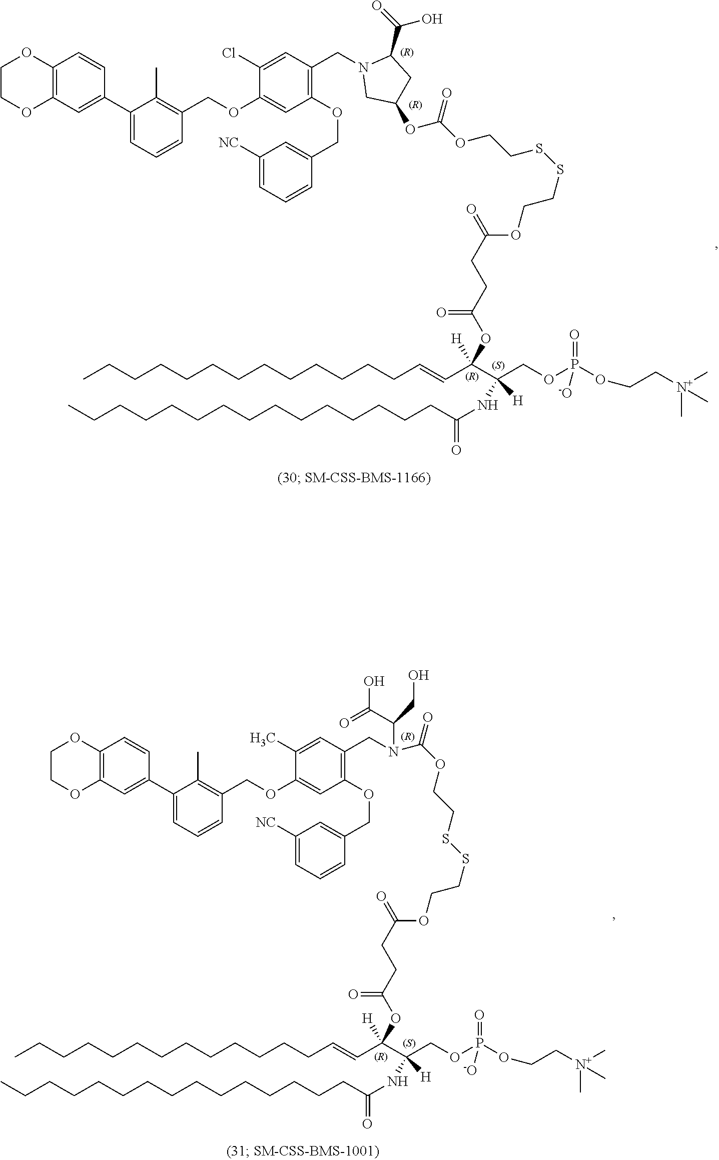

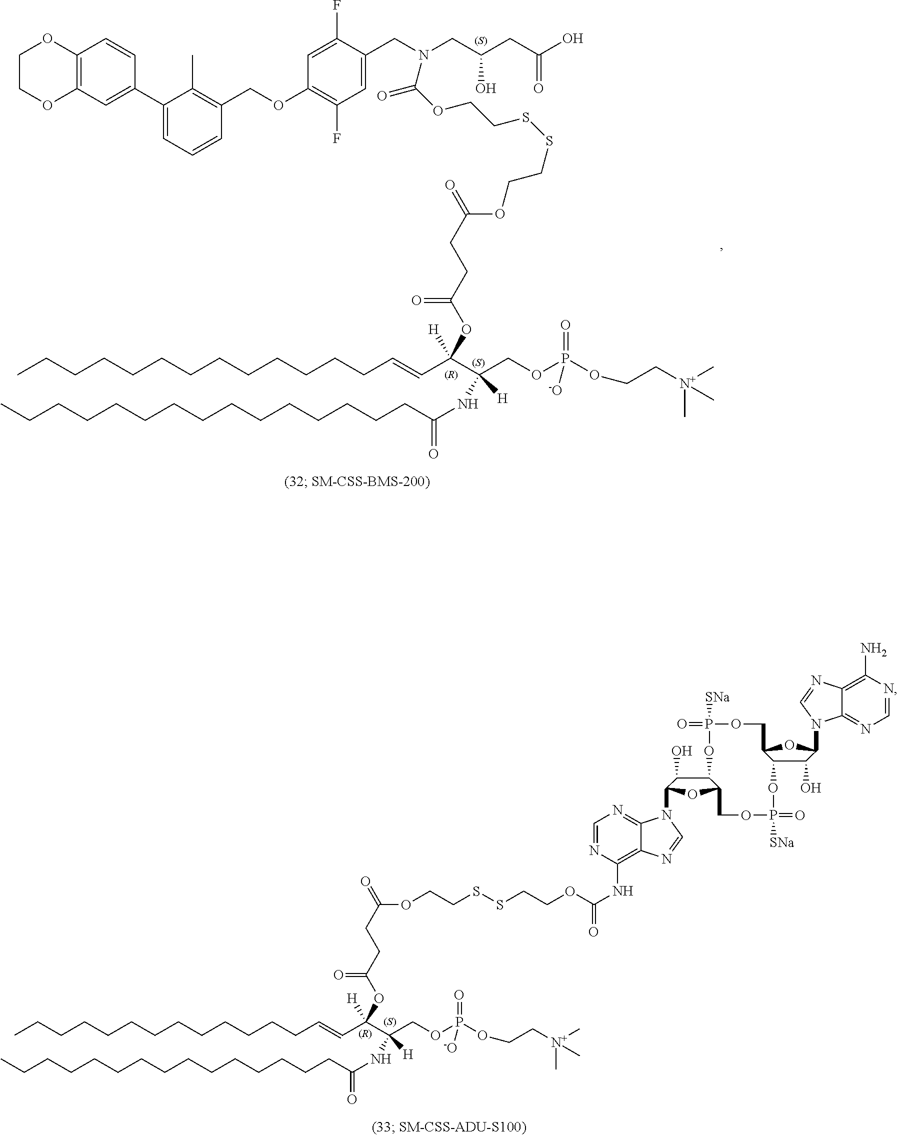

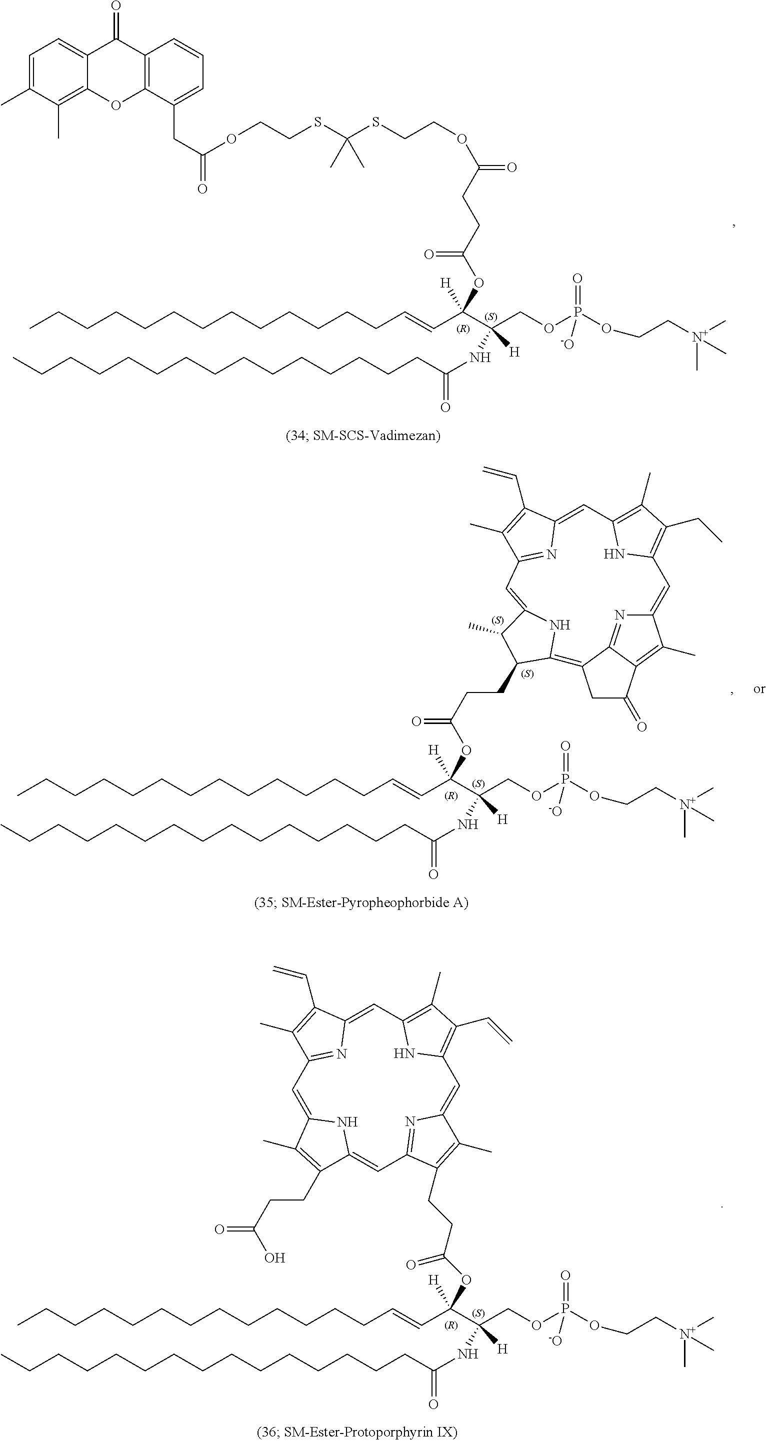

21. The nanovesicle of claim 14, further comprising one or more DOX-drug conjugates in an interior core of the nanovesicle.

22. The nanovesicle of claim 21, wherein the one or more DOX-drug conjugates comprises Formula (VII)-(VII): 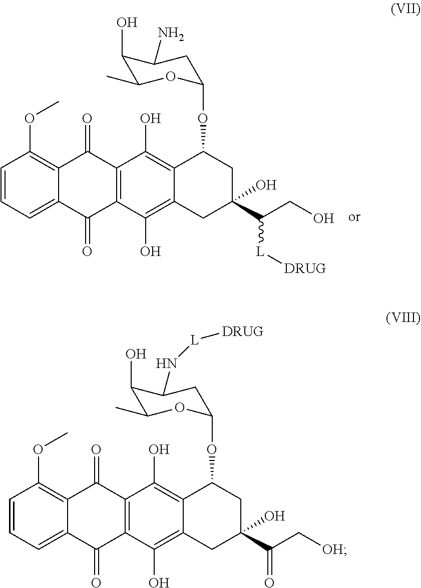
wherein:
L is a linker moiety; and
Drug is an anti-cancer drug.

23. The nanovesicle of claim 22, wherein the anti-cancer drug is hydrophobic or hydrophilic.

24. The nanovesicle of claim 22, wherein anti cancer drug is selected from: indoximod, bortezomib, epacadostat, imiquimod, imatinib, canertinib, ceritinib, dabrafenib, vemurafenib, vorinostat, ADU-S100, amrubicin, AZD4635, BMS-1001, BMS-1166, BMS-200, BMS202, BMS-242, CA170, cabazitaxel, cabozantinib, camptothecin (CPT), capecitabine, carboplatin, cisplatin, dacarbazine, darolutamide, degarelix, digitoxin, digoxin, docetaxel, eribulin, etoposide, 5-fluorouracil, gemcitabine, irinotecan, ixabepilone, lenvatinib, methotrexate, mitoxantrone, NIR178, NLG919, oxaliplatin, paclitaxel, pazopanib, pemetrexed, preladenant, septacidin, SN-38, sorafenib, streptozocin, sunitinib, temozolomide, tipiracil, trifluridine, vadimezan, vinblastine, vincristine, vinorelbine, vipadenant, or combinations thereof.

25. The nanovesicle of claim 21, wherein the one or more DOX-drug conjugate comprises Formula (IX)-(XVIII): 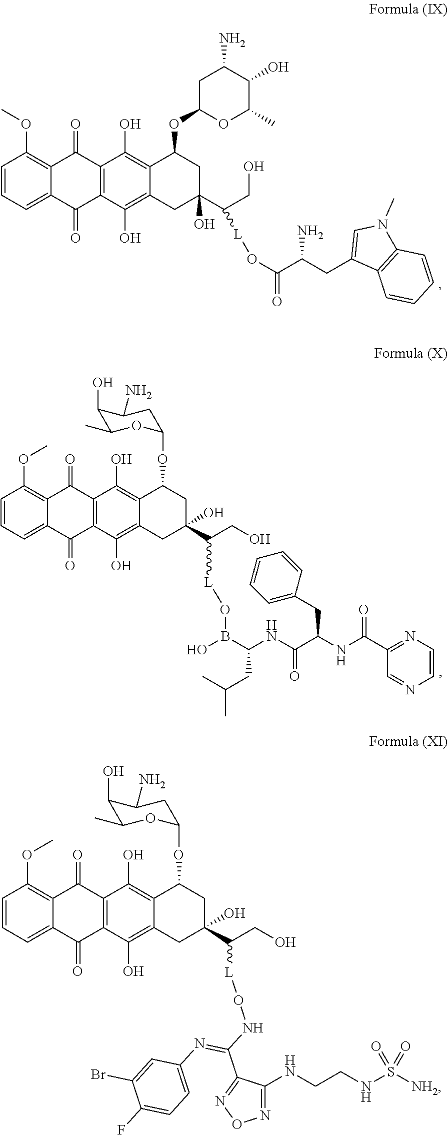 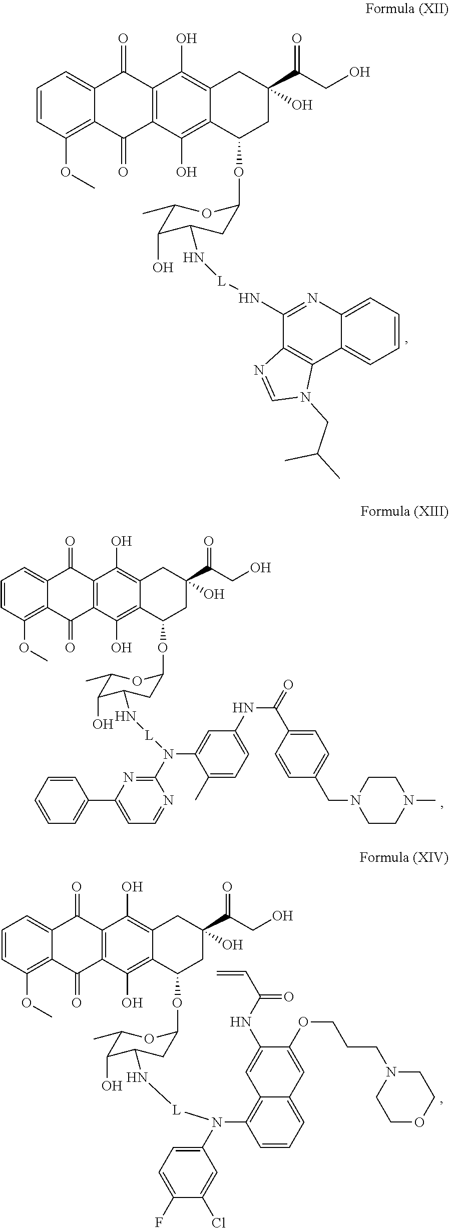 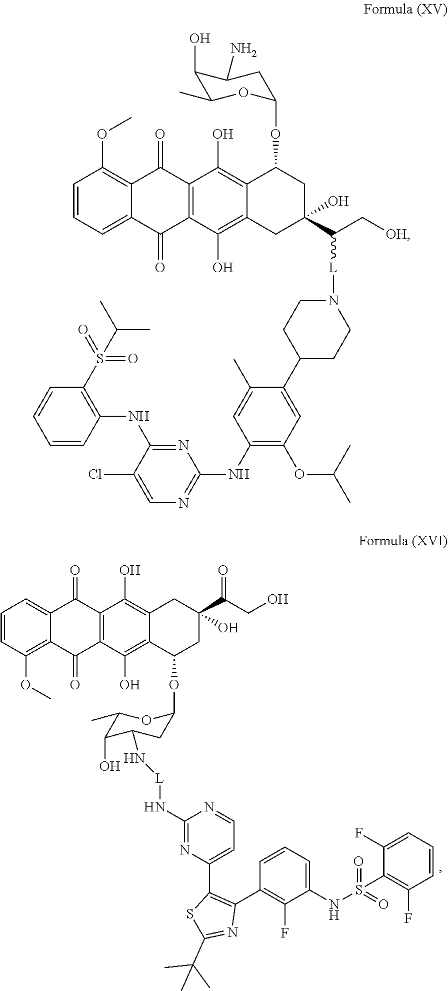 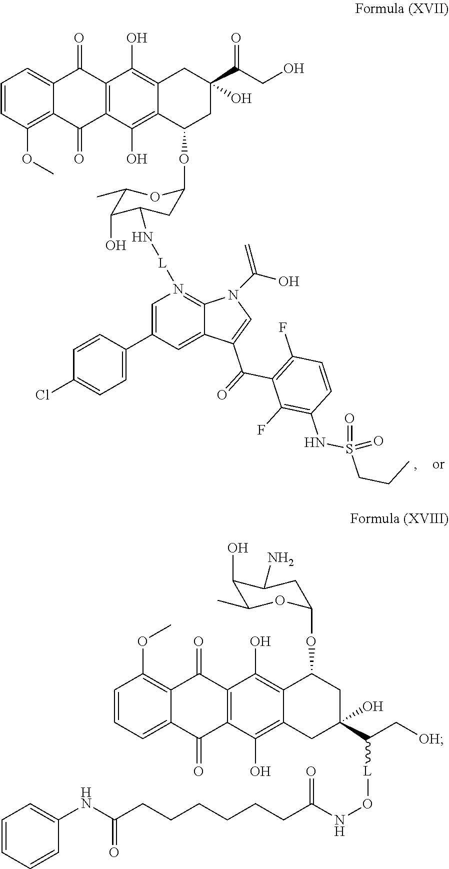
wherein L is a linker moiety.

26. The nanovesicle of claim 22, wherein L is selected from: 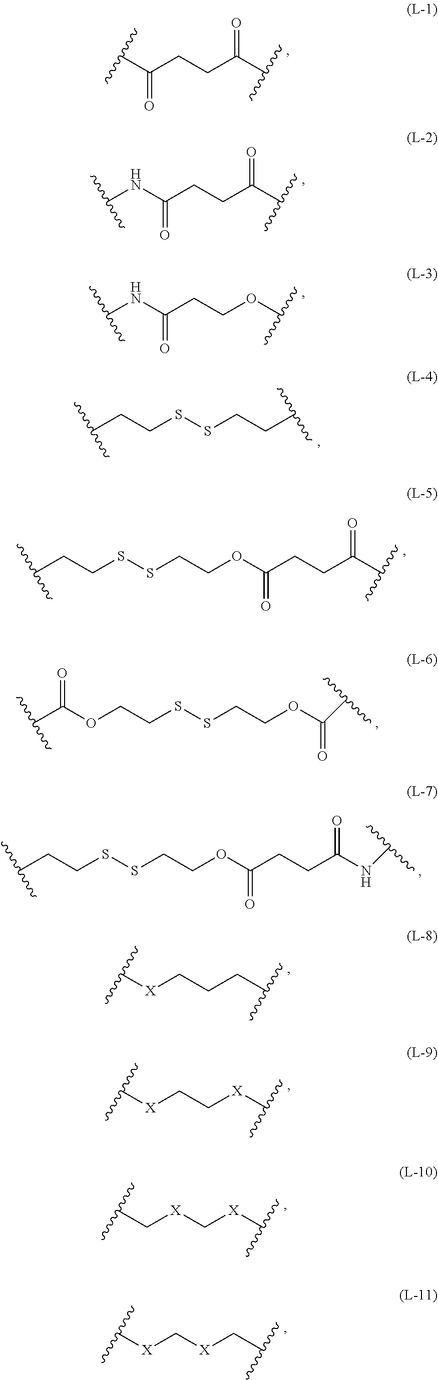 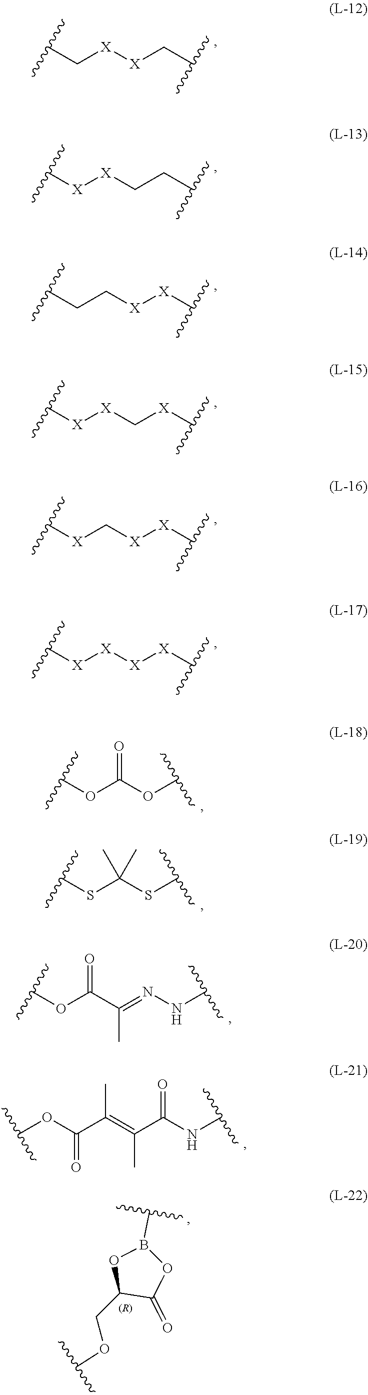 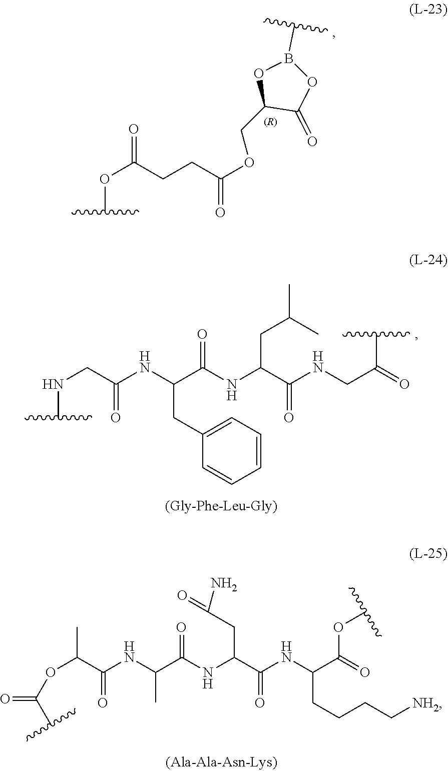
or combinations thereof;
wherein X is independently, O, S, —NH, or —CO.

27. The nanovesicle of claim 21, wherein the one or more DOX-drug conjugates is:

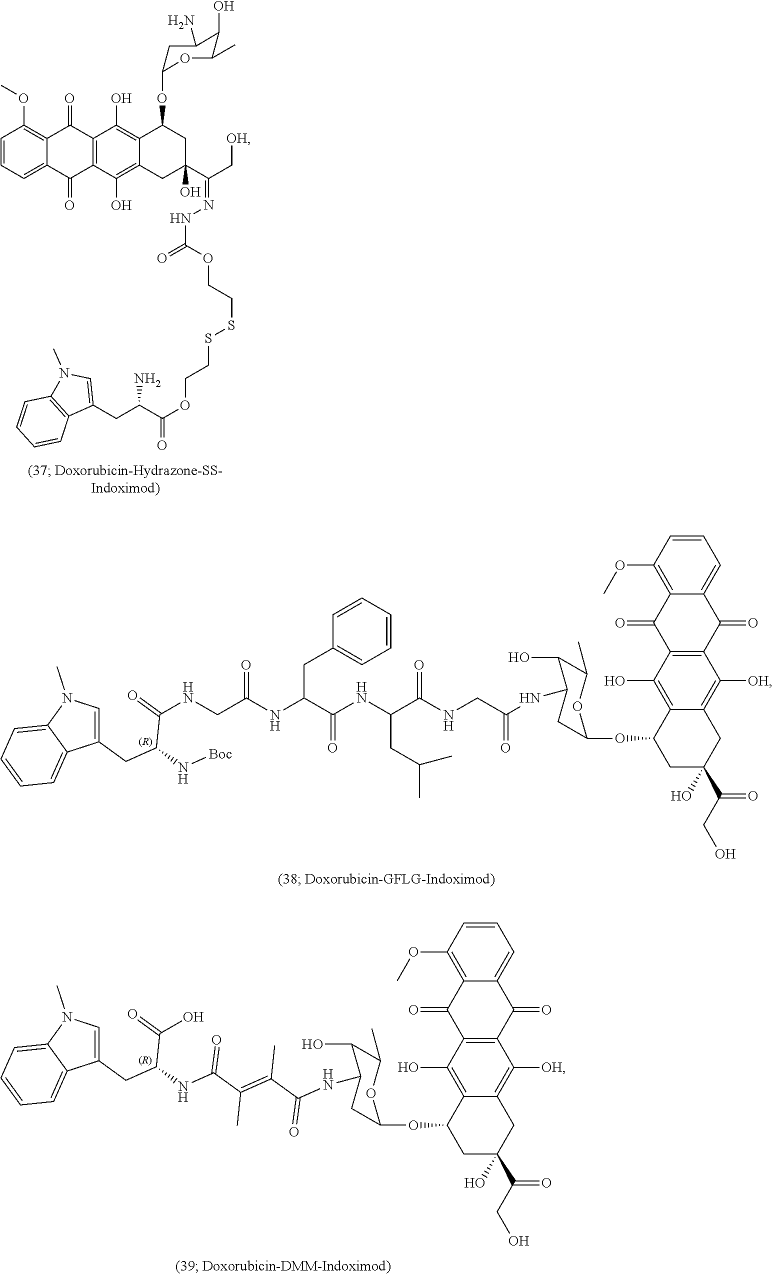

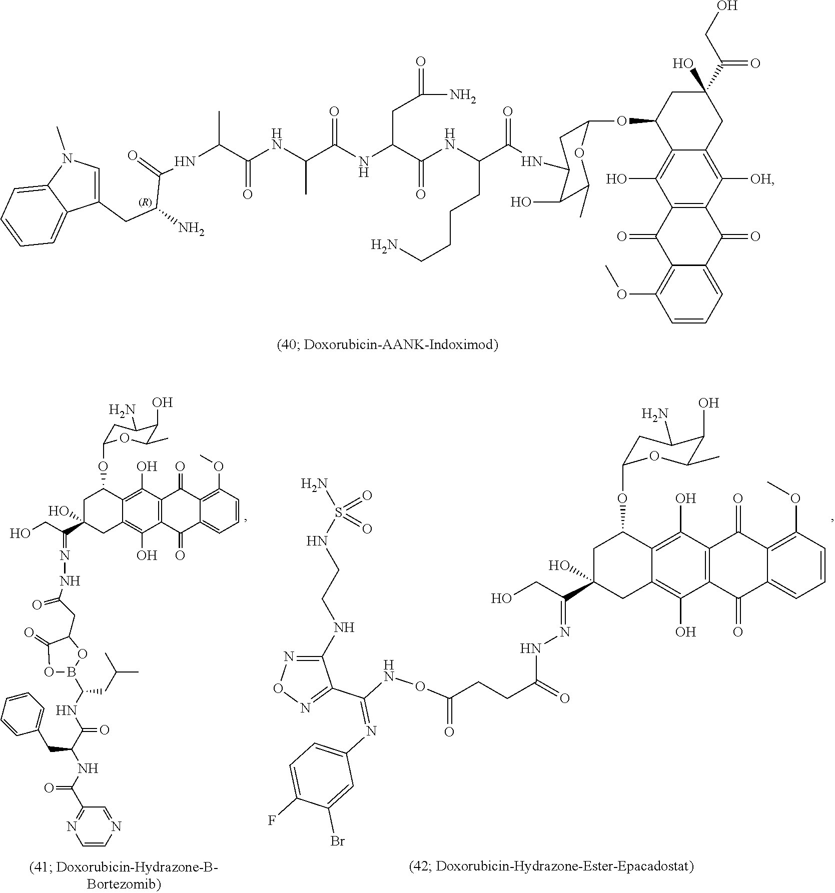

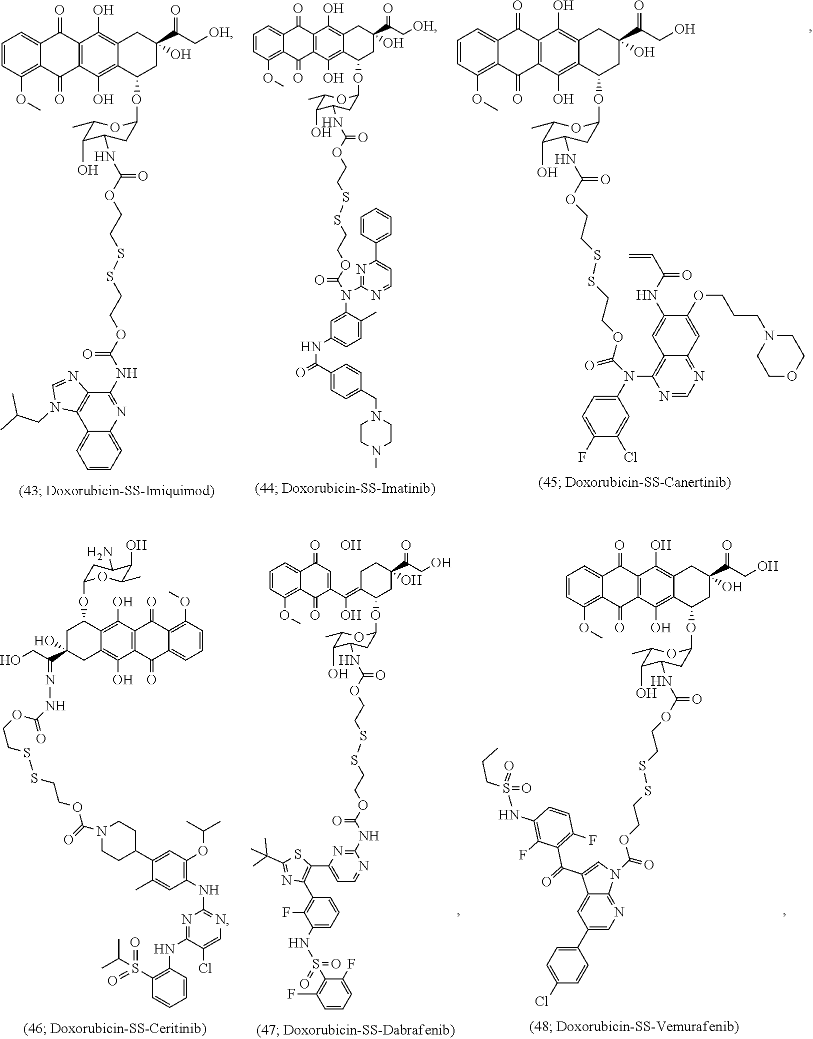

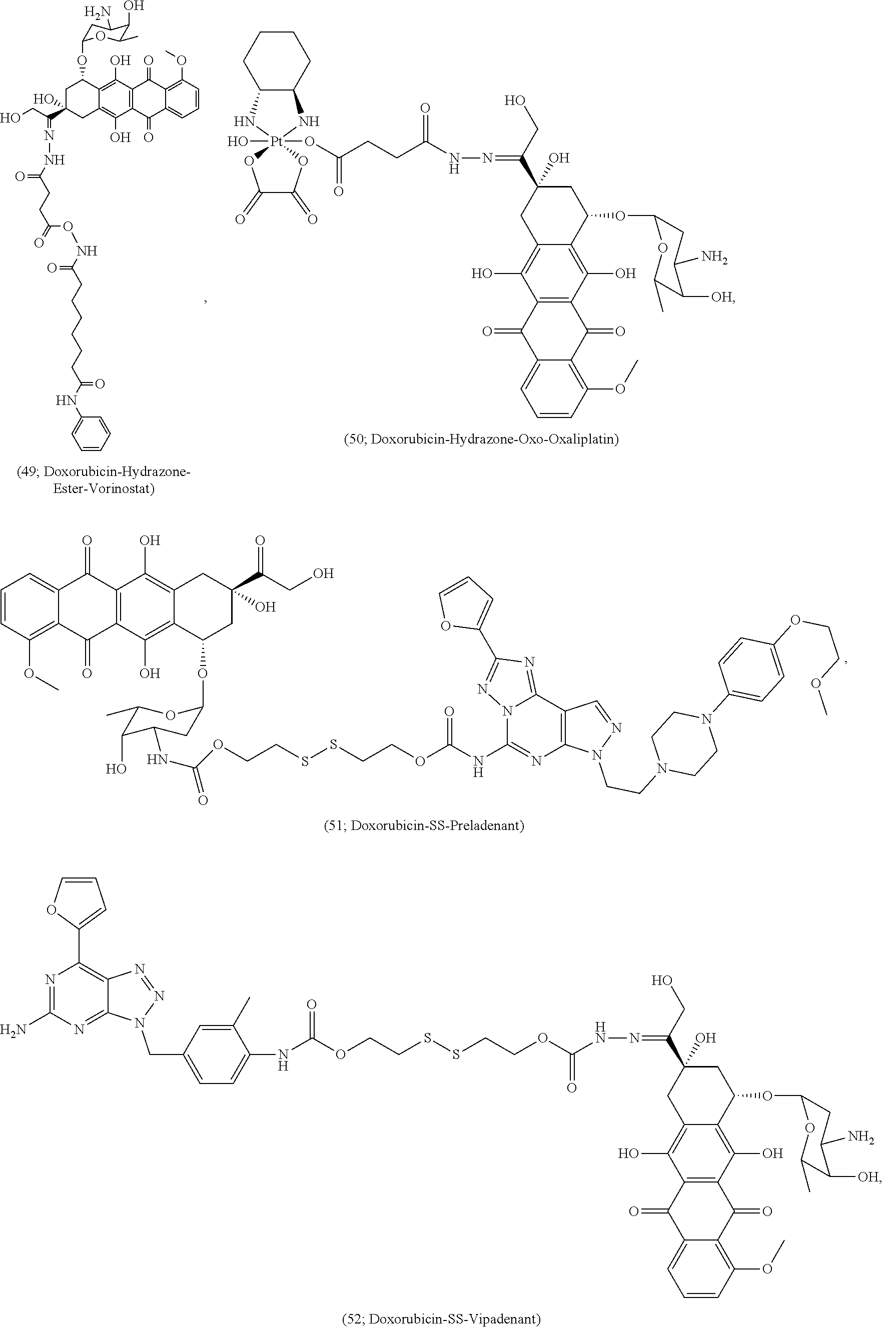

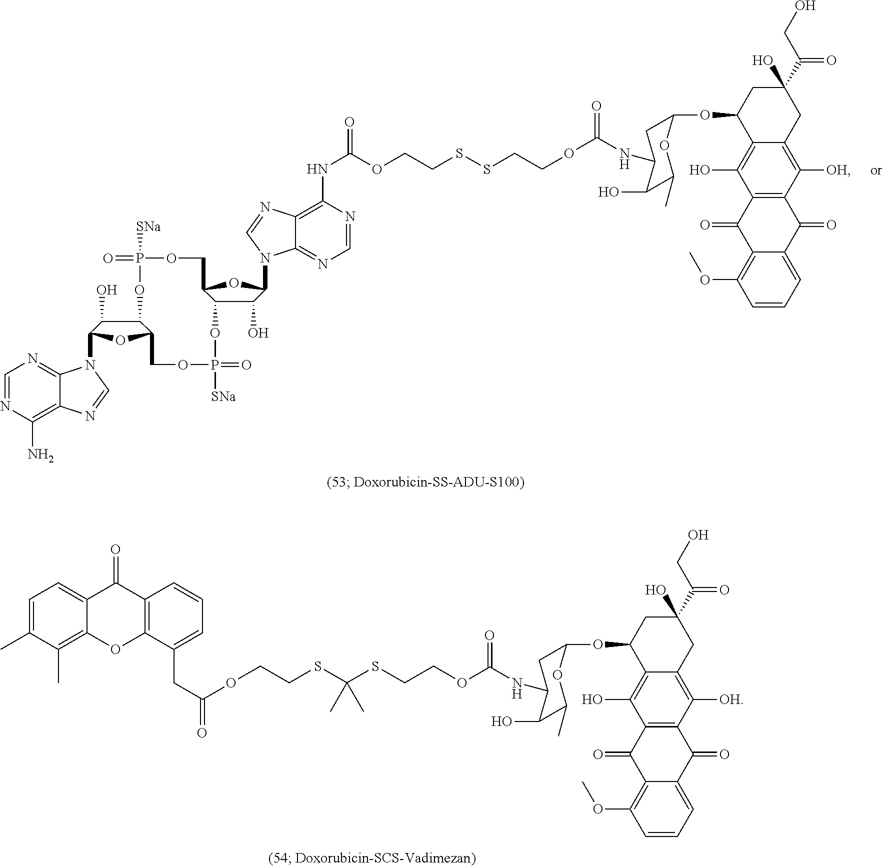

28. The nanovesicle of claim 21, wherein the sphingomyelin-drug conjugate comprises (4 SM-CSS-CPT) and the one or more DOX-drug conjugates comprises (37; Doxorubicin-Hydrazone-SS-Indoximod).

29. The nanovesicle of claim 14, wherein the nanovesicle is further conjugated to one or more tumor targeting ligands.

30. The nanovesicle of claim 29, wherein the one or more tumor targeting ligands is selected from the group consisting of folate or folic acid, anisamide, phenylboronic acid, glycyrrhizic acid, pamidronic acid, triphenylphosphine, flavin mononucleotide; Polysaccharides: hyaluronic acid, galactose, chitosan, mannose, heparin, dextran, N-acetyl-β-D-galactosamine, sialic acid, lactobionic acid; Proteins: transferrin, EGFP-EGF1, AopB, ApoE, lactoferrin, tumor necrosis factor (TNF)-related apoptosis-inducing ligand (TRAIL); Antibodies: intercellular adhesion molecule 1 antibody (ICAM-1), CD44 antibody, EGFR antibody (cetuximab, panitumumab), PD-L1 antibody, EpCAM antibody, EphA10 antibody, AFP antibody, AMG655 antibody; Peptides: arginine-glycine-aspartate (RGD), asparagine-glycine-arginine (NGR), melittin (Mel), MT peptide, T7 peptide, Cell-penetrating peptides (CPP), Gly-Sar, mitochondria) targeting peptide (pALDH Leader), K237 peptide, YIGSR peptide, poly(histidine-arginine)6 (H6R6), angiopep-2, octreotide, pardaxin, Fragment C of tetanus toxin (TTC); Aptamers: aptamer S6, aptamer GBI-10, aptamer AS1411, aptamer RP, aptamer R8, aptamer AraHH036, aptamer MUC1, aptamer PSMA, aptamer EpCAM, and combinations thereof.

31-68. (canceled)

69. A method of treating and/or preventing cancer in a subject in need thereof, the method comprising administering to the subject the nanovesicle of claim 14.

70. The method of claim 69, wherein the cancer is adrenal cancer, anal cancer, basal and squamous cell skin cancer, bile duct cancer, bladder cancer, bone cancer, brain and spinal cord tumors (e.g., astrocytoma, glioblastoma multiforme, meningioma), breast cancer, cervical cancer, colorectal cancer, endometrial cancer, esophagus cancer, Ewing family of tumors, eye cancer (ocular melanoma), gallbladder cancer, gastrointestinal neuroendocrine (carcinoid) tumors, gastrointestinal stromal tumor (gist), gestational trophoblastic disease, Kaposi sarcoma, kidney cancer, laryngeal and hypopharyngeal cancer, liver cancer, lung cancer, lung carcinoid tumor, malignant mesothelioma, melanoma skin cancer, Merkle cell skin cancer, nasal cavity and paranasal sinuses cancer, nasopharyngeal cancer, neuroblastoma, non-small cell lung cancer, neoplasm of the central nervous system (CNS), oral cavity and oropharyngeal cancer, osteosarcoma, ovarian cancer, pancreatic cancer, pancreatic neuroendocrine tumor (net), penile cancer, pituitary tumors, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer, uterine sarcoma, vaginal cancer, vulvar cancer, Waldenstrom macroglobulinemia, Wilms tumor, squamous cell cancer, cancers of unknown primary (CUP), environmentally induced cancers, combinations of the cancers, and metastatic lesions of the cancers. In some embodiments, the cancer is leukemia or lymphoma, for example, lymphoblastic lymphoma or B-cell Non-Hodgkin's lymphoma.

71. The method of claim 69, wherein the cancer is a hematologic malignancy.

72. The method of claim 71, wherein the hematologic malignancy is chronic lymphocytic leukemia (CLL), acute leukemia, acute lymphoid leukemia (ALL), B-cell acute lymphoid leukemia (B-ALL), T-cell acute lymphoid leukemia (T-ALL), T-cell lymphoma, B-cell lymphoma, chronic myelogenous leukemia (CML), acute myelogenous leukemia, B-cell prolymphocytic leukemia, blastic plasmacytoid dendritic cell neoplasm, Burkitt's lymphoma, diffuse large B-cell lymphoma, follicular lymphoma, hairy cell leukemia, small cell follicular lymphoma, large cell follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or preleukemia. In other embodiments, the cancer is a human hematologic malignancy such as myeloid neoplasm, acute myeloid leukemia (AML), AML with recurrent genetic abnormalities, AML with myelodysplasia-related changes, therapy-related AML, acute leukemias of ambiguous lineage, myeloproliferative neoplasm, essential thrombocythemia, polycythemia vera, myelofibrosis (MF), primary myelofibrosis, systemic mastocytosis, myelodysplastic syndromes (MDS), myeloproliferative/myelodysplastic syndromes, chronic myeloid leukemia, chronic neutrophilic leukemia, chronic eosinophilic leukemia, myelodysplastic syndromes (MDS), refractory anemia with ringed sideroblasts, refractory cytopenia with multilineage dysplasia, refractory anemia with excess blasts (type 1), refractory anemia with excess blasts (type 2), MDS with isolated del (5q), unclassifiable MDS, myeloproliferative/myelodysplastic syndromes, chronic myelomonocytic leukemia, atypical chronic myeloid leukemia, juvenile myelomonocytic leukemia, unclassifiable myeloproliferative/myelodysplastic syndromes, lymphoid neoplasms, precursor lymphoid neoplasms, B lymphoblastic leukemia, B lymphoblastic lymphoma, T lymphoblastic leukemia, T lymphoblastic lymphoma, mature B-cell neoplasms, diffuse large B-cell lymphoma, primary central nervous system lymphoma, primary mediastinal B-cell lymphoma, Burkitt lymphoma/leukemia, follicular lymphoma, chronic lymphocytic leukemia, small lymphocytic lymphoma, B-cell prolymphocytic leukemia, lymphoplasmacytic lymphoma, mantle cell lymphoma, marginal zone lymphomas, post-transplant lymphoproliferative disorders, HIV-associated lymphomas, primary effusion lymphoma, intravascular large B-cell lymphoma, primary cutaneous B-cell lymphoma, hairy cell leukemia, multiple myeloma, monoclonal gammopathy of unknown significance (MGUS), smoldering multiple myeloma, or solitary plasmacytomas (solitary bone and extramedullary).

73. The method of claim 69, wherein the cancer comprises a solid tumor.

74. The method of claim 73, wherein the solid tumor selected from the group consisting of lung cancer, colorectal cancer, breast cancer, pancreatic cancer, gallbladder cancer, brain and spinal cord cancer, head and neck cancer, skin cancers, testicular cancer, prostate cancer, ovarian cancer, renal cell carcinoma (RCC), bladder cancer. and hepatocellular carcinoma (HCC).

75. The method of claim 69, wherein the nanovesicle is present in a pharmaceutical composition.


SEQUENCE LISTING (4 sequences)

SEQ ID NO: 1 - DNA, 19 bp, Artificial Sequence
  Synthetic
agccagagtccttcagaga

SEQ ID NO: 2 - DNA, 20 bp, Artificial Sequence
  Synthetic
tccttagccactccttctgt

SEQ ID NO: 3 - DNA, 19 bp, Artificial Sequence
  Synthetic
aatggtgaaggtcggtgtg

SEQ ID NO: 4 - DNA, 24 bp, Artificial Sequence
  Synthetic
gtggagtcatactggaacatgtag